(19) United States
(12) Patent Application Publication
(10) Pub. No.: US 2024/0199649 A1
(43) Pub. Date: Jun. 20, 2024

(54) 4-AMINOPYRROLO[2,1-F][1,2,4]TRIAZINES AND PREPARATION AND USES THEREOF

(71) Applicant: Biosplice Therapeutics, Inc., San Diego, CA (US)

(72) Inventors: Gopi Kumar Mittapalli, San Diego, CA (US); Chi Ching Mak, San Diego, CA (US); Lewis Daniel Turner, San Diego, CA (US); Ramkrishna Reddy Vakiti, San Diego, CA (US); Brian Walter Eastman, San Diego, CA (US); Brian Joseph Hofilena, San Diego, CA (US)

(21) Appl. No.: 18/501,307

(22) Filed: Nov. 3, 2023

Related U.S. Application Data
(60) Provisional application No. 63/494,875, filed on Apr. 7, 2023.
(60) Provisional application No. 63/422,202, filed on Nov. 3, 2022.

(51) Int. Cl. C07D 519/00; C07B 59/00

(52) U.S. Cl. CPC C07D 519/00; C07B 59/002; C07B 2200/05

(57) ABSTRACT

4-Aminopyrrolo[2,1-f][1,2,4]triazine compounds for treating various diseases and pathologies are disclosed. More particularly, the present disclosure concerns the use of 4-aminopyrrolo[2,1-f][1,2,4]triazine compounds or analogs thereof, in the treatment of disorders characterized by overexpression of DYRK1A (e.g., cancer, Down syndrome, Alzheimer's disease, diabetes, and osteoarthritis).

### RELATED APPLICATIONS

This application claims the benefit of U.S. Provisional Application Nos. 63/422,202, filed Nov. 3, 2022, and 63/494,875, filed Apr. 7, 2023, which are incorporated herein by reference in their entirety.

### TECHNICAL FIELD

This disclosure relates to inhibitors of dual-specificity tyrosine phosphorylation-regulated 1A kinase, and compositions comprising the same. More particularly, it concerns the use of a 4-aminopyrrolo[2,1-f][1,2,4]triazine compound or salts or analogs thereof, in the treatment of disorders characterized by the abnormal expression and/or activity of DYRK1A (e.g., cancer, Down syndrome, Alzheimer's disease, diabetes, and osteoarthritis).

### BACKGROUND

Dual-specificity tyrosine phosphorylation-regulated kinases (DYRK1A, 1B, 2-4) comprise a family of protein kinases within the CMGC group of the eukaryotic kinome. These protein kinases are involved in multiple cellular functions, including intracellular signaling, mRNA splicing, chromatin transcription, DNA damage repair, cell survival, cell cycle control, differentiation, homocysteine/methionine/folate regulation, body temperature regulation, endocytosis, neuronal development, synaptic plasticity, etc. Abnormal expression and/or activity of some of these kinases, DYRK1A in particular, is seen in many human nervous system diseases, such as cognitive deficits associated with Down syndrome, Alzheimer's disease, and related diseases, tauopathies, dementia, Pick's disease, Parkinson's disease, and other neurodegenerative diseases, Phelan-McDermid syndrome, autism, and CDKL5 deficiency disorder. DYRKs are also involved in diabetes, abnormal folate/methionine metabolism, osteoarthritis, several solid cancers (glioblastoma, breast, and pancreatic cancers) and leukemias (acute lymphoblastic leukemia, acute megakaryoblastic leukemia), viral infections (influenza, HIV-1, HCMV, HCV, CMV, HPV), as well as infections caused by unicellular parasites (Leishmania, Trypanosoma, Plasmodium) (International Journal of Molecular Sciences (2021), 22(11), 6047). DYRK1A has also been identified as a critical stabilizer of EGFR (Cell Death & Disease (2019), 10, 282) which is a crucial factor contributing to the keratinization, cell hyperproliferation, abnormal differentiation and inflammatory infiltration during the progress of psoriasis.

### SUMMARY

The present disclosure provides methods and reagents, involving contacting a cell with an agent, such as a 4-aminopyrrolo[2,1-f][1,2,4]triazine compound, in a sufficient amount to antagonize DYRK1A activity, e.g., reduced the proliferation of head and neck squamous cell carcinoma, luminal/HER2 breast cancer (Cell (2016), 164(1-2), 293-309) or pancreatic adenocarcinoma, as well as impaired the self-renewal capacity of glioblastoma and compromised ovarian cancer spheroid cell viability (Molecular Cancer Research (2017), 15(4), 371-381).

The present disclosure also provides methods and reagents, involving contacting a cell with an agent, such as a 4-aminopyrrolo[2,1-f][1,2,4]triazine compound, in a sufficient amount to antagonize DYRK1A activity, e.g., i) to normalize prenatal and early postnatal brain development; ii) to improve cognitive function in youth and adulthood; and/or iii) to attenuate Alzheimer's-type neurodegeneration.

Some embodiments disclosed herein include DYRK1A inhibitors containing a 4-aminopyrrolo[2,1-f][1,2,4]triazine core. Other embodiments disclosed herein include pharmaceutical compositions and methods of treatment using these compounds.

One embodiment disclosed herein includes a compound having the structure of Formula I.

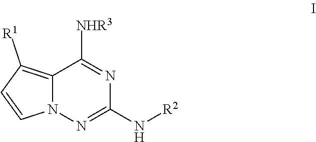

or a pharmaceutically acceptable salt thereof,

wherein,
R¹ is (9-10 membered heteroaryl) optionally substituted with 1-10 R⁴;
R² is selected from the group consisting of -heterocyclyl optionally substituted with 1-10 R⁵ and -carbocyclyl optionally substituted with 1-12 R⁶;
R³ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl);
each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁷, —(C₁₋₅ alkylene)ₚOR⁸, and —C(═O)R⁹, wherein the —(C₁₋₅ alkylene) is optionally substituted with 1-5 halide and/or 1-3 unsubstituted —(C₁₋₃ alkyl);
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —C(═O)R¹¹, and —NHC(═O)R¹²;
each R⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁸ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁰ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)ₚOR⁸;
each R¹¹ is —N(R¹³)²;
each R¹² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl); each R¹³ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1; and
wherein each H atom is optionally, independently replaced by ²H (D) (deuterium).

In another embodiment disclosed herein includes a compound having the structure of Formula I:

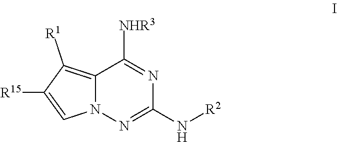

wherein:
R¹ is (9-10 membered heteroaryl) optionally substituted with 1-10 R⁴;
R² is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁵ and —(C₁₋₅ alkylene)ₚcarbocyclyl optionally substituted with 1-12 R⁶, wherein the —(C₁₋₅ alkylene) is optionally substituted with 1-5 halide and/or 1-3 unsubstituted —(C₁₋₃ alkyl);
R³ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R¹⁶;
each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl);
each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁷, -carbocyclyl optionally substituted with 1-12 R¹⁴, —(C₁₋₅ alkylene)ₚOR⁸, and —C(═O)R⁹, wherein the —(C₁₋₅ alkylene) is optionally substituted with 1-5 halide and/or 1-3 unsubstituted —(C₁₋₃ alkyl);
alternatively, two R⁵ attached to the same carbon atom are taken together to form a carbonyl group;
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —(C₁₋₅ alkylene)ₚOR¹⁰, —C(═O)R¹¹, —NHC(═O)R¹², and -carbocyclyl optionally substituted with 1-12 R¹⁴, wherein the —(C₁₋₅ alkylene) is optionally substituted with 1-5 halide and/or 1-3 unsubstituted —(C₁₋₃ alkyl);
each R⁷ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, and -carbocyclyl optionally substituted with 1-12 R⁴;
each R⁸ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁰ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)ₚOR⁸;
each R¹¹ is —N(R¹³)₂;
each R¹² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹³ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹⁵ is selected from the group consisting of H and halide;
each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1; and
wherein each H atom is optionally, independently replaced by ²H (D) (deuterium).

In another embodiment disclosed herein includes a compound having the structure of Formula I:

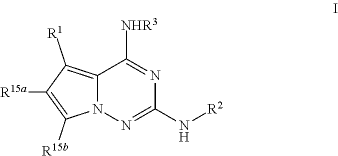

wherein:
R¹ is (9-10 membered heteroaryl) optionally substituted with 1-10 R⁴;
R² is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁵ and —(C₁₋₅ alkylene)ₚcarbocyclyl optionally substituted with 1-12 R⁶, wherein the —(C₁₋₅ alkylene) is optionally substituted with 1-5 halide and/or 1-3 unsubstituted —(C₁₋₃ alkyl);
R³ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R¹⁶;
each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl);
each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁷, -carbocyclyl optionally substituted with 1-12 R¹⁴, —(C₁₋₅ alkylene)ₚOR⁸, and —C(═O)R⁹, wherein the —(C₁₋₅ alkylene) is optionally substituted with 1-5 halide and/or 1-3 unsubstituted —(C₁₋₃ alkyl);
alternatively, two R⁵ attached to the same carbon atom are taken together to form a carbonyl group;
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —(C₁₋₅ alkylene)ₚOR¹⁰, —C(═O)R¹¹, —NHC(═O)R¹², and -carbocyclyl optionally substituted with 1-12 R¹⁴, wherein the —(C₁₋₅ alkylene) is optionally substituted with 1-5 halide and/or 1-3 unsubstituted —(C₁₋₃ alkyl);
each R⁷ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, and -carbocyclyl optionally substituted with 1-12 R¹⁴;
each R⁸ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁰ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)ₚOR⁸;
each R¹¹ is —N(R¹³)₂;
each R¹² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹³ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R^{15a} is selected from the group consisting of H, D, and halide;
R^{15b} is selected from the group consisting of H and D;
each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1; and
wherein each H atom is optionally, independently replaced by ²H (D) (deuterium).

Some embodiments include stereoisomers and pharmaceutically acceptable salts of a compound of Formula (I). Some embodiments include pharmaceutically acceptable salts of a compound of Formula (I).

Some embodiments include pro-drugs of a compound of Formula (I).

Some embodiments of the present disclosure include pharmaceutical compositions comprising a compound of Formula (I) and a pharmaceutically acceptable carrier, diluent, or excipient.

Other embodiments disclosed herein include methods of inhibiting DYRK1A by administering to a patient affected by a disorder or disease in which DYRK1A overexpression is implicated, such as Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor and Stroke.

Inhibitors of DYRK1A can also be used to treat tauopathies. Tauopathies are neurodegenerative disorders characterized by the deposition of abnormal tau protein in the brain. The spectrum of tau pathologies expands beyond the traditionally discussed disease forms like Pick's disease, progressive supranuclear palsy, corticobasal degeneration, and argyrophilic grain disease. Emerging entities and pathologies include globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia (Handbook of Clinical Neurology (2018), 145, 355-368 and Aging Cell (2019), 18(5), e13000).

Inhibitors of DYRK1A can also be used to treat disorders associated with abnormal folate/methionine metabolism.

Non-limiting examples of diseases which can be treated with the compounds and compositions provided herein include a variety of cancers, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

Some embodiments of the present disclosure include methods to prepare compounds of Formula (I).

It is to be understood that both the foregoing general description and the following detailed description are exemplary and explanatory only and are not restrictive of the disclosure, as claimed.

### DETAILED DESCRIPTION

Provided herein are compositions and methods for inhibiting DYRK1A.

Some embodiments provided herein relate to a method for treating a disease including, but not limited to, neurological diseases or disorders, cancers, cognitive deficits, knee osteoarthritis, tendinopathy, viral infections, unicellular parasite infections, and motor deficits.

In some embodiments, non-limiting examples of a neurological disease or disorder which can be treated with the compounds and compositions provided herein include, but are not limited to, Alzheimer's disease, amyotrophic lateral sclerosis, Down Syndrome, frontotemporal dementia with Parkinsonism-17 (FTDP-17), Lewy body dementia, Parkinson's disease, Pick's disease tauopathies, and additional diseases with pronounced neurodegeneration such as autism, dementia, epilepsy, Huntington's disease, multiple sclerosis; diseases and disorders associated with acquired brain injury such as Chronic Traumatic Encephalopathy, Traumatic Brain Injury, Tumor, and Stroke.

In some embodiments, non-limiting examples of cancers which can be treated with the compounds and compositions provided herein include solid cancers (e.g., glioblastoma, ovarian, breast, and pancreatic cancers) and leukemias (e.g., acute lymphoblastic leukemia, acute megakaryoblastic leukemia, and chronic myeloid leukemia).

In some embodiments, pharmaceutical compositions are provided that are effective for treatment of a disease of an animal, e.g., a mammal, caused by DYRK1A overexpression. The composition includes a pharmaceutically acceptable carrier and a compound as described herein.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-O— group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “haloalkoxy” means a haloalkyl-O— group in which the haloalkyl group is as described herein. Exemplary haloalkoxy groups include fluoromethoxy, difluoromethoxy, trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylalkylene” means an aryl-alkylene- group in which the aryl and alkylene moieties are as previously described. In some embodiments, arylalkylene groups contain a C₁₋₄alkylene moiety. Exemplary arylalkylene groups include benzyl and 2-phenethyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched alkyl, alkenyl or alkynyl substituted with one or more chloro, bromo, fluoro, and/or iodo atom(s). In some embodiments, a haloalkyl is a fluoroalkyl, wherein one or more of the hydrogen atoms have been substituted by fluoro. In some embodiments, haloalkyls are 1 to 3 carbons in length (e.g., 1 to 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings such as bicyclic and spirocyclic heterocyclyls. Heterocyclyls may be substituted or unsubstituted with one or more substituents. In some embodiments, heterocycles have 3-11 members. In six membered monocyclic heterocycles, the heteroatom(s) are selected from one to three of O, N and S, and wherein when the heterocycle is five membered, it can have one or two heteroatoms selected from O, N, and S. Examples of heterocyclyl include 3-azabicyclo[3.2.1]octane, 2-azaspiro[3.5]nonanyl, 7-azaspiro[3.5]nonane, azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, 1,4-dioxaspirodecanyl, morpholinyl, thiomorpholinyl, piperazinyl, pyranyl, pyrrolidinyl, tetrahydrofuryl, tetrahydropyridinyl, octahydrocyclopenta[c]pyrrolyl, oxazinyl, 1-oxaspiro[3.5]nonanyl, 2-oxaspiro[3.5]nonanyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 3-7 members. In six membered monocyclic heterocycles, the heteroatom(s) are selected from one to three of O, N and S, and wherein when the heterocycle is five membered, it can have one or two heteroatoms selected from O, N, and S. Examples of monocyclic heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents. In some embodiments, bicyclic heterocycles have 4-11 members with the heteroatom(s) being selected from one to five of O, N and S. Examples of bicyclic heterocyclyls include 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 3-azabicyclo[3.2.1]octane, 2-azabicyclo[2.2.2]octane, and the like.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N and S. Examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R¹¹; —NRR′; —C(O)NRR′; —C(NR)NR′R″; —C(NR′)R¹¹; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with 1-3 R″′; 5-10 membered heteroaryl having from 1-4 heteroatoms independently selected from N, O, and S and optionally substituted with 1-3 R″′; C₃₋₇ carbocyclyl optionally substituted with 1-3 R″′; and 3-8 membered heterocyclyl having from 1-4 heteroatoms independently selected from N, O, and S and optionally substituted with 1-3 R″′; wherein each R″′ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formula I wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), isotopes of carbon, such as ¹¹C, ¹³C and ¹⁴C, isotopes of chlorine, such as ³⁶Cl, isotopes of fluorine, such as ¹⁸F, isotopes of iodine, such as ¹²³I and ¹²⁵I, isotopes of nitrogen, such as ¹³N and ¹⁵N, isotopes of oxygen, such as ¹⁵O, ¹⁷O and ¹⁸O, isotopes of phosphorus, such as ³²P, and isotopes of sulfur, such as ³⁵S.

The term “administration” or “administering” refers to a method of providing a dosage of a compound or pharmaceutical composition to a vertebrate or invertebrate, including a mammal, a bird, a fish, or an amphibian, where the method of administration is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The terms “pharmaceutically acceptable carrier”, “pharmaceutically acceptable diluent” and “pharmaceutically acceptable excipient” include any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Brunton et al. (Eds.) (2017); Goodman and Gilman's: The Pharmacological Basis of Therapeutics, 13th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formula I in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age, and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing, or preventing the further development of a disorder, and/or reducing the severity of symptoms that will or are expected to develop.

“Drug-eluting” and/or controlled release as used herein refers to any and all mechanisms, e.g., diffusion, migration, permeation, and/or desorption by which the drug(s) incorporated in the drug-eluting material pass therefrom overtime into the surrounding body tissue.

“Drug-eluting material” and/or controlled release material as used herein refers to any natural, synthetic, or semi-synthetic material capable of acquiring and retaining a desired shape or configuration and into which one or more drugs can be incorporated and from which incorporated drug(s) are capable of eluting overtime.

“Elutable drug” as used herein refers to any drug or combination of drugs having the ability to pass over time from the drug-eluting material in which it is incorporated into the surrounding areas of the body.

### Compounds

The compounds and compositions described herein can be used to inhibit DYRK1A for treating a disorder or disease in which DYRK1A overexpression is implicated, such as in neurological diseases or disorders, cancers, cognitive deficits, knee osteoarthritis, tendinopathy, viral infections, unicellular parasite infections, and motor deficits.

Some embodiments of the present disclosure include compounds of Formula I:

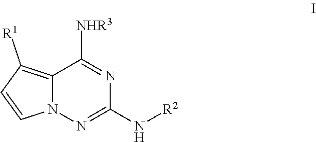

or salts, pharmaceutically acceptable salts, or prodrugs thereof.

Some embodiments of the present disclosure include compounds of Formula I:

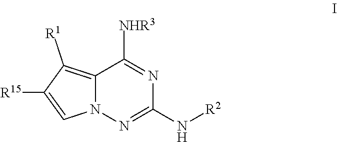

or salts, pharmaceutically acceptable salts, or prodrugs thereof.

Some embodiments of the present disclosure include compounds of Formula I:

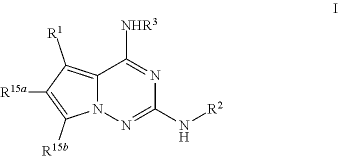

or salts, pharmaceutically acceptable salts, or prodrugs thereof.

In some embodiments of Formula I, R¹ is (9-10 membered heteroaryl) optionally substituted with 1-10 R⁴. In some embodiments, R¹ is (9-10 membered heteroaryl) optionally substituted with 1-10, 1-9, 1-8, 1-7, 1-6, 1-5, 1-4, 1-3, 1-2, or 1 R⁴.

In some embodiments of Formula I, R¹ is selected from the heteroaryl group consisting of:

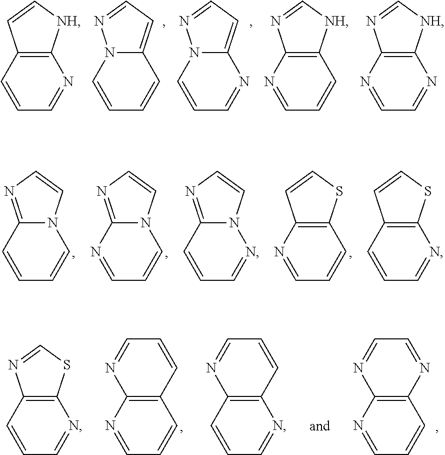

optionally substituted with 1-10 (e.g., 1-9, 1-8, 1-7, 1-6, 1-5, 1-4, 1-3, 1-2, 1) R⁴.

In some embodiments of Formula I, R¹ is selected from the heteroaryl group consisting of:

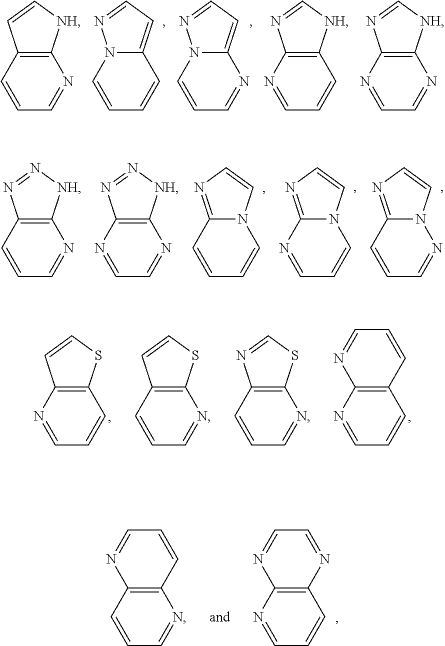

optionally substituted with 1-10 (e.g., 1-9, 1-8, 1-7, 1-6, 1-5, 1-4, 1-3, 1-2, 1) R⁴.

In some embodiments of Formula I, R¹ is selected from the heteroaryl group consisting of:

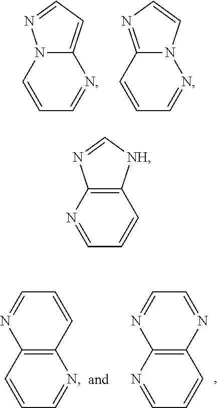

optionally substituted with 1-10 (e.g., 1-9, 1-8, 1-7, 1-6, 1-5, 1-4, 1-3, 1-2, 1) R⁴.

In some embodiments of Formula I, R¹ is selected from the heteroaryl group consisting of:

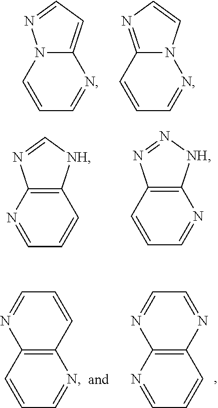

optionally substituted with 1-10 (e.g., 1-9, 1-8, 1-7, 1-6, 1-5, 1-4, 1-3, 1-2, 1) R⁴.

In some embodiments of Formula I, R¹ is selected from the heteroaryl group consisting of:

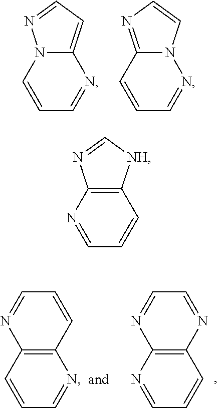

optionally substituted with 1-3 (e.g., 1-2, 1) R⁴.

In some embodiments of Formula I, R¹ is selected from the heteroaryl group consisting of

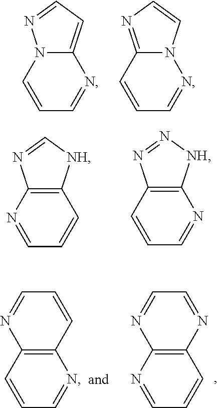

optionally substituted with 1-3 (e.g., 1-2, 1) R⁴.

In some embodiments of Formula I, R¹ is selected from the group consisting of:

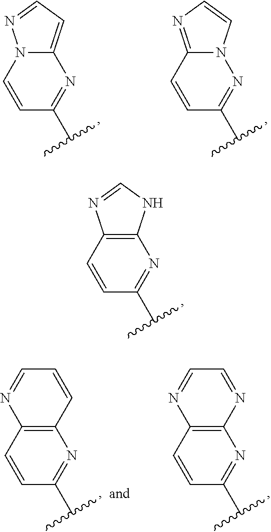

optionally substituted with 1-3 R⁴.

In some embodiments of Formula I, R¹ is selected from the group consisting of:

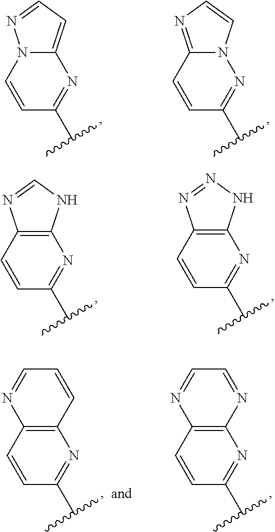

optionally substituted with 1-3 R⁴.

In some embodiments of Formula I, R¹ is selected from the group consisting of:

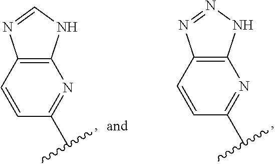

optionally substituted with 1-2 R⁴.

In some embodiments of Formula I, R¹ is selected from the group consisting of:

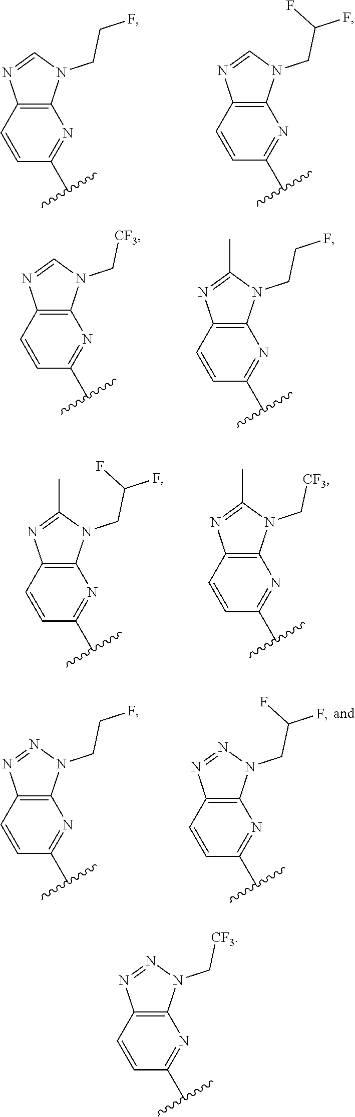

In some embodiments of Formula I, R² is selected from the group consisting of -heterocyclyl optionally substituted with 1-10 (e.g., 1-9, 1-8, 1-7, 1-6, 1-5, 1-4, 1-3, 1-2, 1) R⁵, and -carbocyclyl optionally substituted with 1-12 (e.g., 1-11, 1-10, 1-9, 1-8, 1-7, 1-6, 1-5, 1-4, 1-3, 1-2, 1) R.

In some embodiments of Formula I, R² is selected from the group consisting of -heterocyclyl optionally substituted with 1-4 (e.g., 1-3, 1-2, 1) R⁵, and -carbocyclyl optionally substituted with 1-4 (e.g., 1-3, 1-2, 1) R⁶.

In some embodiments of Formula I, R² is -carbocyclyl optionally substituted with 1-3 R⁶, wherein the carbocyclyl is selected from the group consisting of cyclopropyl, cyclobutyl, cyclopentyl, and cyclohexyl.

In some embodiments of Formula I, R² is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁵ and —(C₁₋₅ alkylene)ₚcarbocyclyl optionally substituted with 1-12 R⁶, wherein the —(C₁₋₅ alkylene) is optionally substituted with 1-5 halide and/or 1-3 unsubstituted —(C₁₋₃ alkyl).

In some embodiments of Formula I, R² is selected from the group consisting of unsubstituted —(C₁₋₄ alkyl), unsubstituted —(C₂₋₄ alkenyl), unsubstituted —(C₂₋₄ alkynyl), unsubstituted —(C₁₋₄ haloalkyl), -heterocyclyl optionally substituted with 1-4 R⁵ and —(C₁₋₂ alkylene)ₚcarbocyclyl optionally substituted with 1-4 R⁶, wherein the —(C₁₋₂ alkylene) is optionally substituted with 1-2 halide (e.g., F, Cl) and/or 1 unsubstituted —(C₁₋₂ alkyl).

In some embodiments of Formula I, R² is selected from the group consisting of:

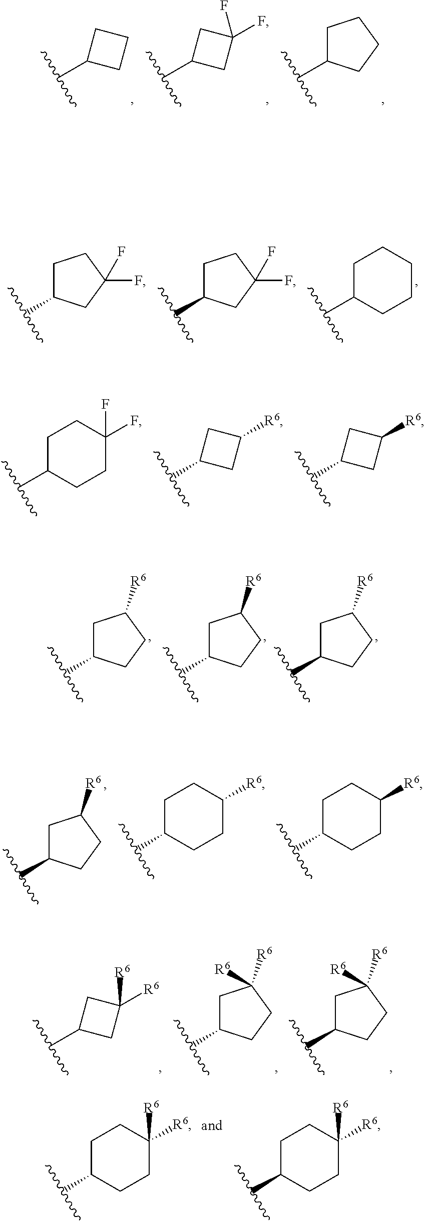

wherein each R⁶ is independently selected from the group consisting of F, Me, —CH₂F, —CHF₂, —CF₃, —CH₂OH, —CH₂OMe, —OH, —OMe, —OEt, —OCD₃, —OCF₃, —OCH₂CH₂F, —OCH₂CHF₂, —OCH₂CF₃, —OCH₂CH₂OMe, —OCH₂CH₂OH, —C(═O)NHMe, —C(═O)NMe₂, and —NHC(═O)Me.

In some embodiments of Formula I, R² is selected from the group consisting of:

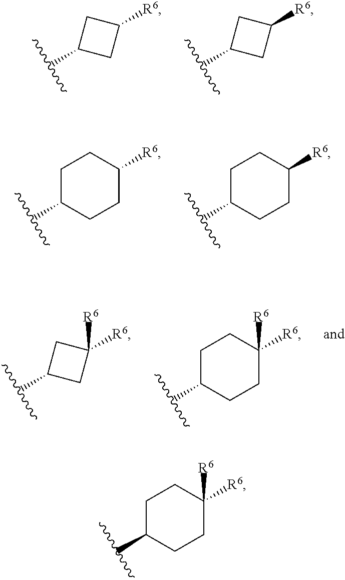

wherein each R⁸ is independently selected from the group consisting of F, Me, —CHF₂, —CH₂OMe, —OH, —OMe, —OCD₃, —OCH₂CHF₂, and —OCH₂CH₂OMe.

In some embodiments of Formula I, R² is -heterocyclyl optionally substituted with 1-3 R⁵, wherein the heterocyclyl is selected from the group consisting of 3-azabicyclo[3.1.0]hexane, 3-azabicyclo[3.2.1]octane, 7-azaspiro[3.5]nonane, 2-azaspiro[3.5]nonane, oxetanyl, tetrahydrofuranyl, tetrahydropyranyl, azetidinyl, pyrrolidinyl, piperidinyl, morpholinyl, piperazinyl, 3-oxabicyclo[3.1.0]hexane, 1-oxaspiro[3.5]nonane, and oxaspiro[3.3]heptanyl.

In some embodiments of Formula I, R² is selected from the group consisting of:

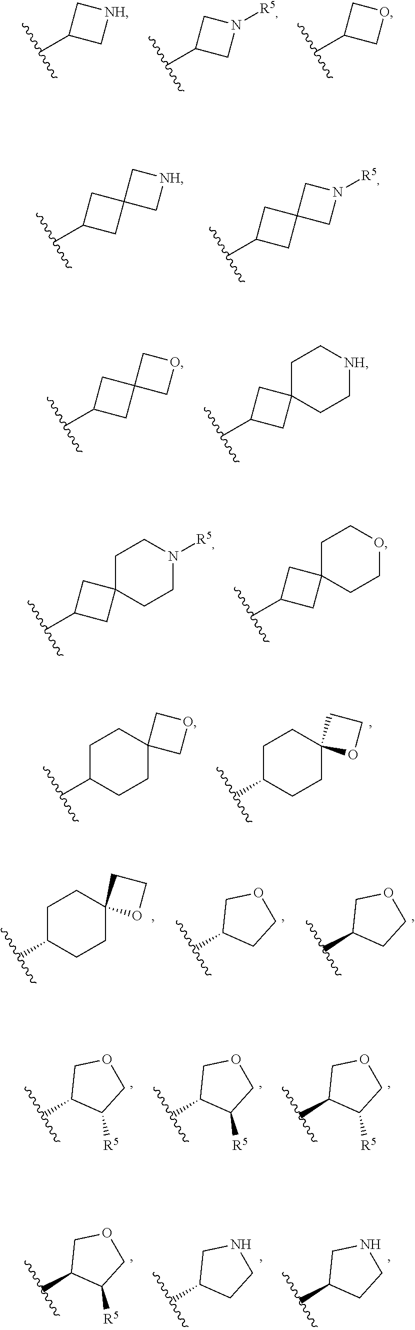

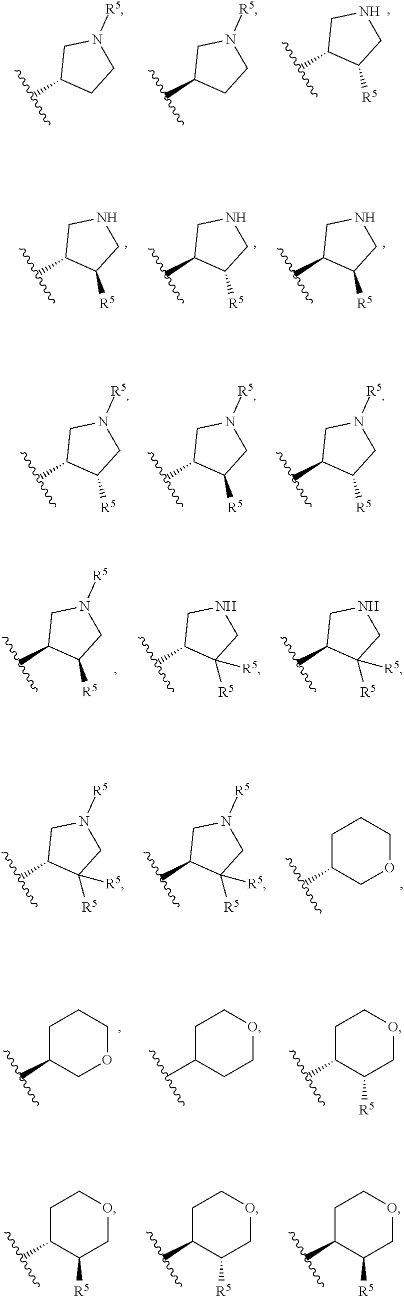

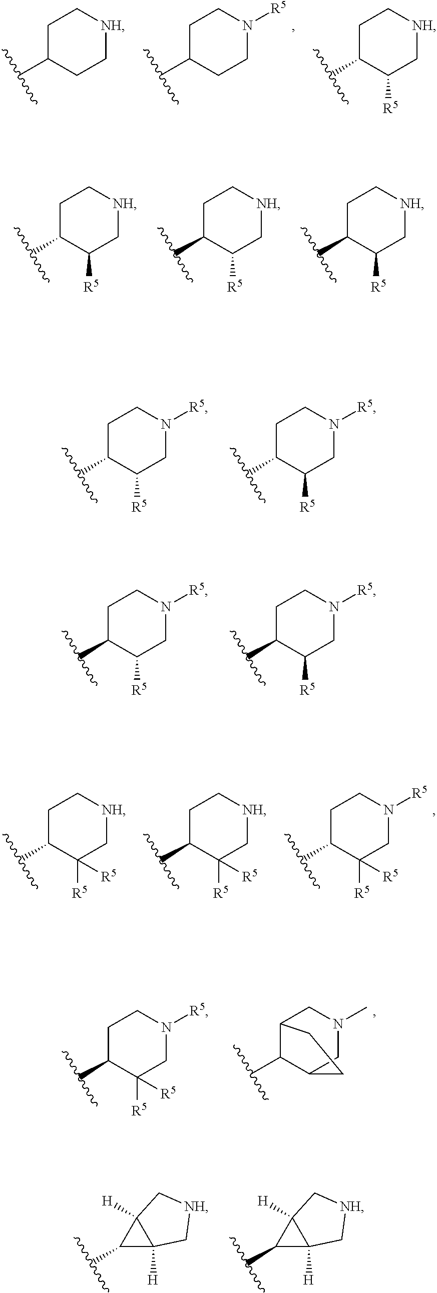

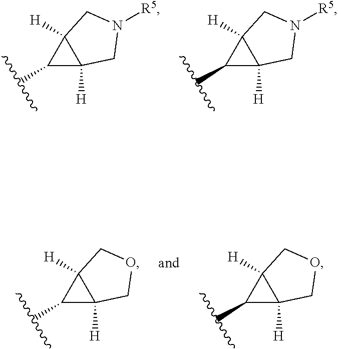

wherein each R⁵ is independently selected from the group consisting of F, Me, Et, iPr, iBu, —CH₂F, —CHF₂, —CF₃, —CH₂OH, —CH₂OMe, —OH, —OMe, —OEt, —OCD₃, —OCF₃, —CH₂CH₂F, —CH₂CHF₂, —CH₂CF₃, —CH₂CH₂CF₃, —CH₂CH₂OMe, —CH₂CH₂OH, —C(═O)Me, —C(═O)Et, —C(═O)iPr, and

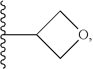 [SMILES: *C1COC1]

with the proviso that F, —OH, —OMe, —OEt, —OCD₃, and —OCF₃ are not attached to N.

In some embodiments of Formula I, R² is selected from the group consisting of:

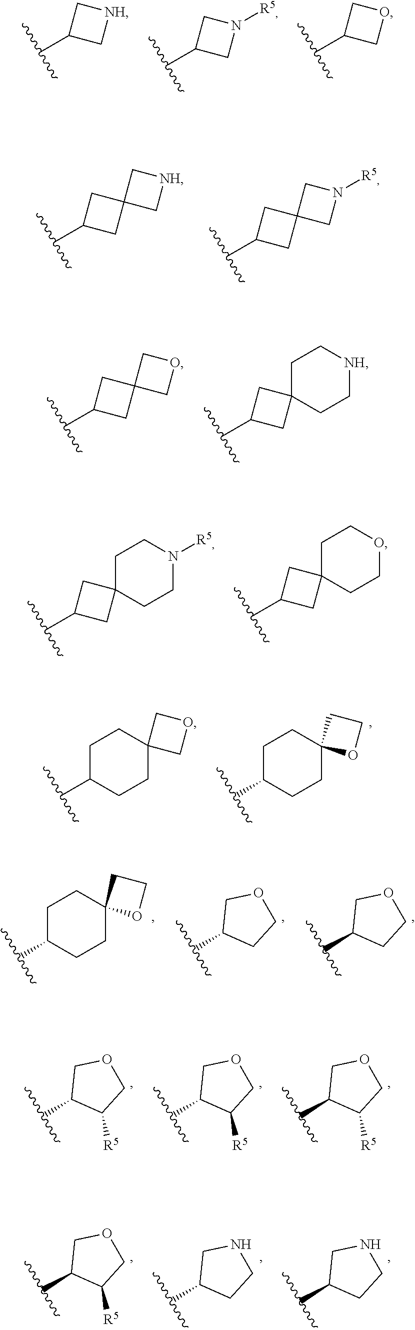

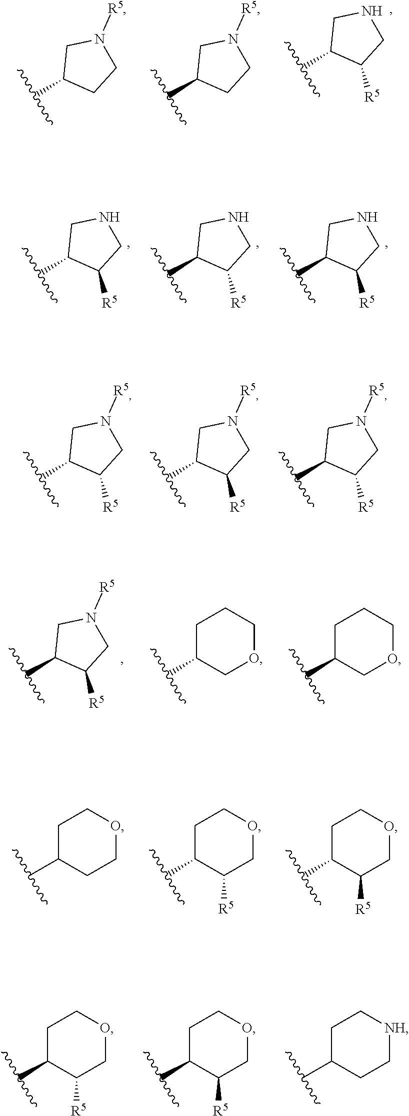

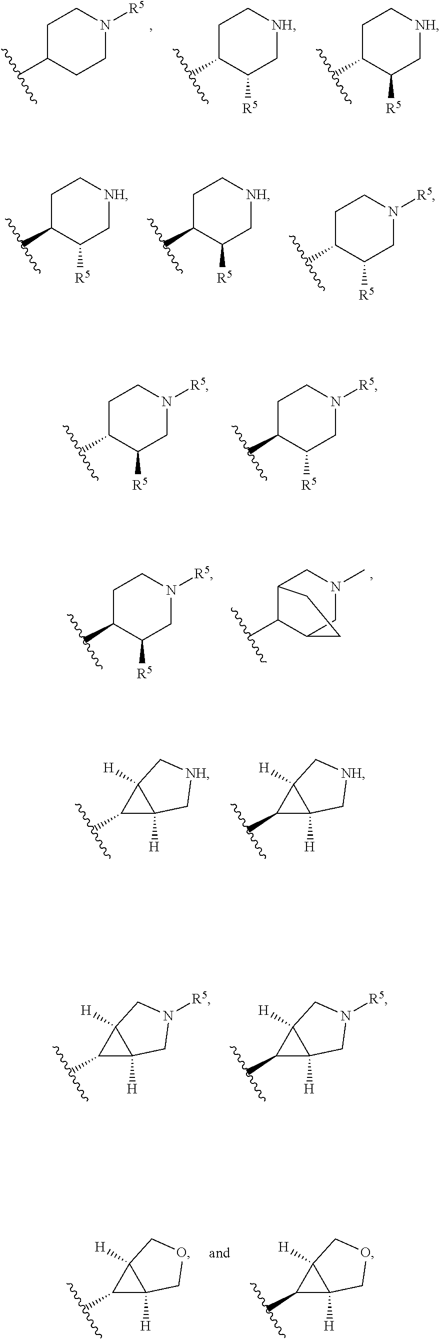

wherein each R⁵ is independently selected from the group consisting of F, Me, Et, iPr, iBu, —CH₂F, —CHF₂, —CF₃, —CH₂OH, —CH₂OMe, —OH, —OMe, —OEt, —OCD₃, —OCF₃, —CH₂CH₂F, —CH₂CHF₂, —CH₂CF₃, —CH₂CH₂CF₃, —CH₂CH₂OMe, —CH₂CH₂OH, —C(═O)Me, —C(═O)Et, —C(═O)iPr, and

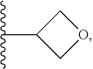 [SMILES: *C1COC1]

with the proviso that F, —OH, —OMe, —OEt, —OCD₃, and —OCF₃ are not attached to N.

In some embodiments of Formula I, R² is selected from the group consisting of:

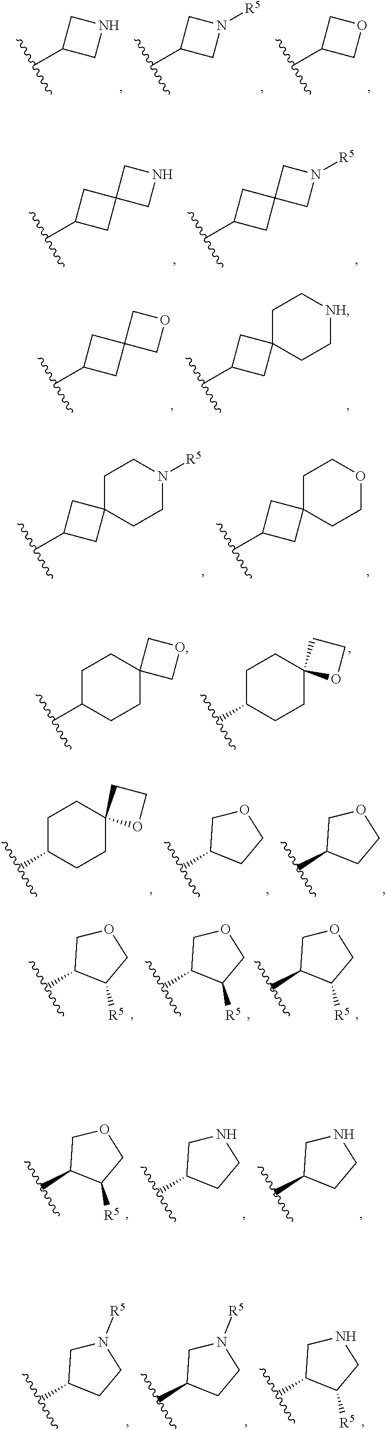

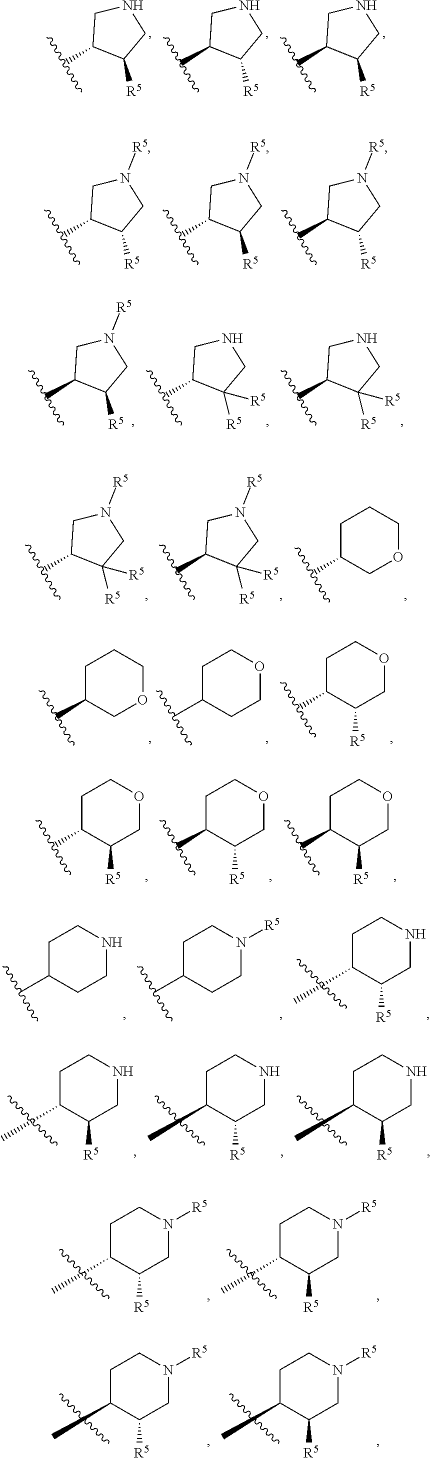

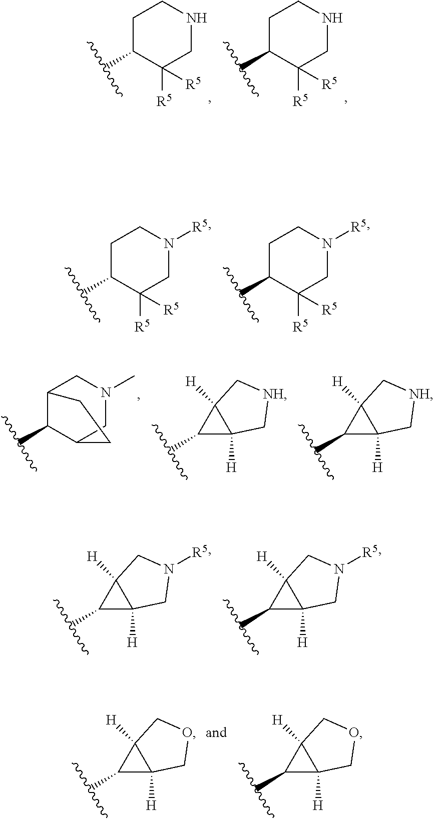

wherein each R⁵ is independently selected from the group consisting of F, Me, Et, iPr, iBu, —CH₂F, —CHF₂, —CF₃, —CH₂OH, —CH₂OMe, —OH, —OMe, —OEt, —OCD₃, —OCF₃, —CH₂CH₂F, —CH₂CHF₂, —CH₂CF₃, —CH₂CH₂CF₃, —CH₂CH₂OMe, —CH₂CH₂OH, —C(═O)Me, —C(═O)Et, —C(═O)iPr, and

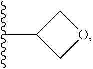 [SMILES: *C1COC1]

with the proviso that F, —OH, —OMe, —OEt, —OCD₃, and —OCF₃ are not attached to N

In some embodiments of Formula I, R² is selected from the group consisting of:

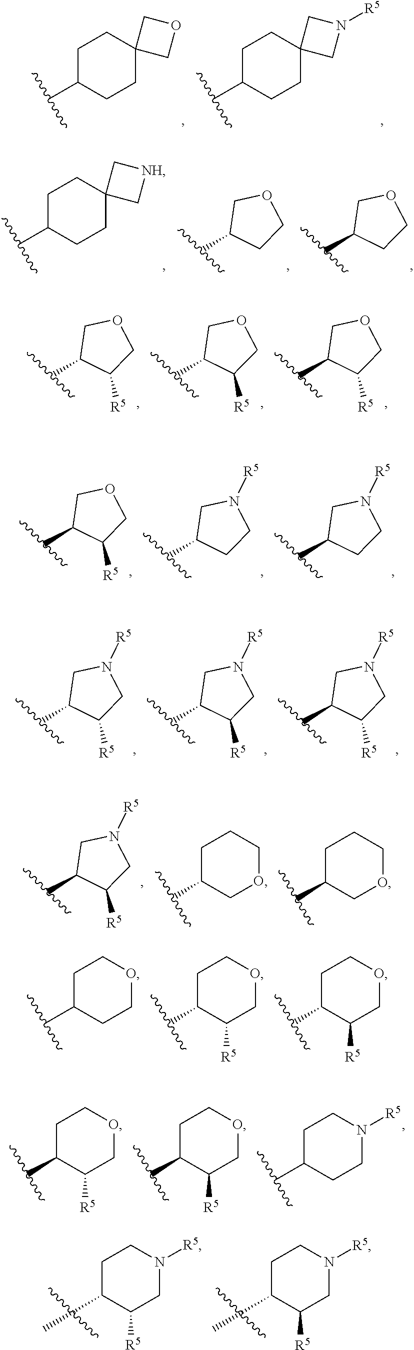

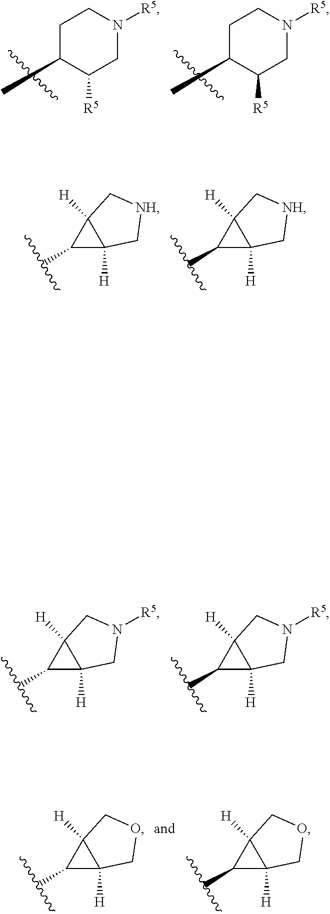

wherein each R⁵ is independently selected from the group consisting of F, Me, iBu, —OH, —OMe, —CH₂CH₂F, —CH₂CHF₂, —CH₂CF₃, —CH₂CH₂CF₃, —CH₂CH₂OMe, —CH₂CH₂OH, —C(═O)Me,

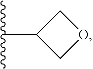 [SMILES: *C1COC1]

with the proviso that F, —OH, —OMe are not attached to N.

In some embodiments of Formula I, R² is selected from the group consisting of:

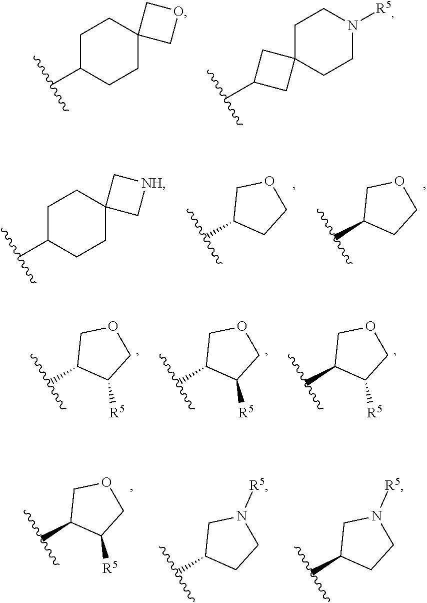

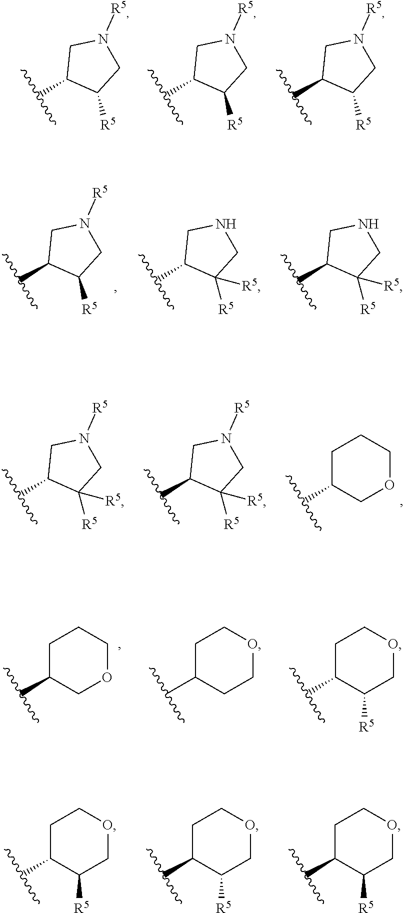

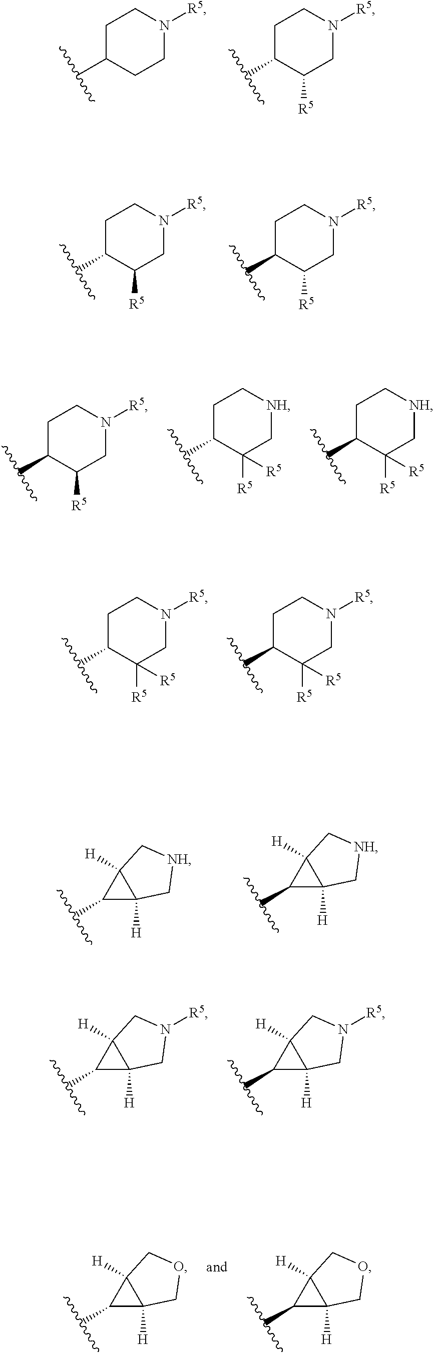

wherein each R⁵ is independently selected from the group consisting of F, Me, iBu, —OH, —OMe, —CH₂CH₂F, —CH₂CHF₂, —CH₂CF₃, —CH₂CH₂CF₃, —CH₂CH₂OMe, —CH₂CH₂OH, —C(═O)Me,

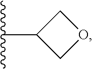 [SMILES: *C1COC1]

with the proviso that F, —OH, —OMe are not attached to N.

In some embodiments of Formula I, R² is selected from the group consisting of:

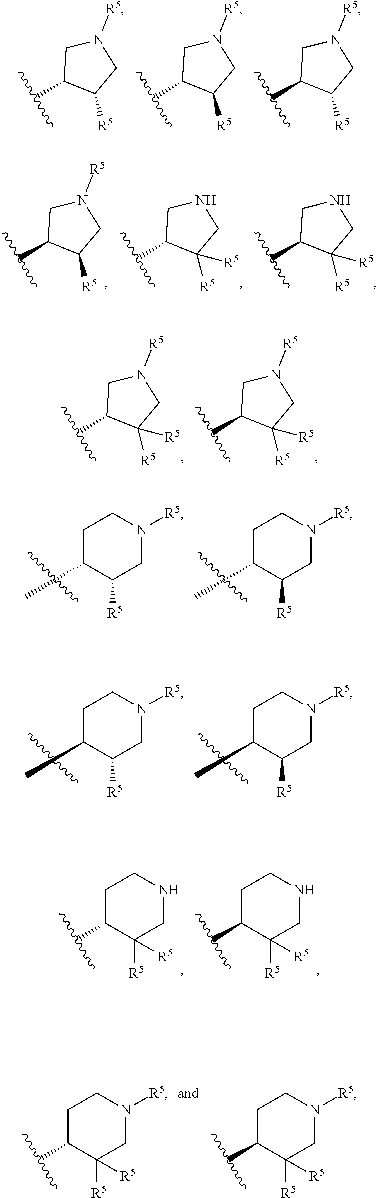

wherein each R⁵ is independently selected from the group consisting of F, Me, —C(═O)Me,

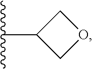 [SMILES: *C1COC1]

with the proviso that F is not attached to N.

In some embodiments of Formula I, R² is selected from the group consisting of:

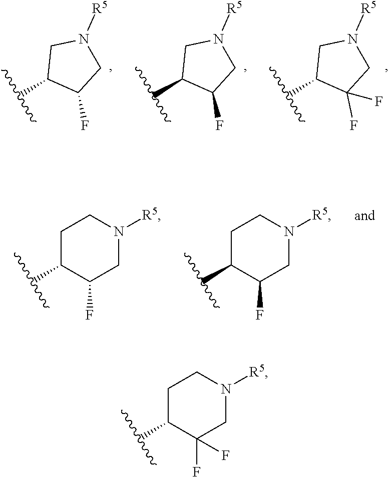

wherein each R⁵ is selected from the group consisting of Me, —C(═O)Me, and

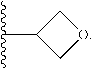 [SMILES: *C1COC1]

In some embodiments of Formula I, R³ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, R³ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

In some embodiments of Formula I, R³ is selected from the group consisting of H, unsubstituted —(C₁₋₃ alkyl), and unsubstituted —(C₁₋₃ haloalkyl).

In some embodiments of Formula I, R³ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R¹⁶.

In some embodiments of Formula I, R³ is selected from the group consisting of H, unsubstituted —(C₁₋₃ alkyl), unsubstituted —(C₂₋₃ alkenyl), unsubstituted —(C₂₋₃ alkynyl), unsubstituted —(C₁₋₃ haloalkyl), and unsubstituted -(4-membered heterocyclyl).

In some embodiments of Formula I, R³ is unsubstituted —(C₁₋₃ alkyl).

In some embodiments of Formula I, R³ is Me.

In some embodiments of Formula I, R³ is H.

In some embodiments of Formula I, each R⁴ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl);

In some embodiments of Formula I, each R⁴ is independently selected from the group consisting of halide (e.g., F, Cl), unsubstituted —(C₁₋₄ alkyl), and unsubstituted —(C₁₋₄ haloalkyl).

In some embodiments of Formula I, each R⁴ is independently selected from the group consisting of fluoro, chloro, methyl, ethyl, isopropyl, 2-fluoroethyl, and 2,2-difluoroethyl.

In some embodiments of Formula I, each R⁵ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 (e.g., 1-9, 1-8, 1-7, 1-6, 1-5, 1-4, 1-3, 1-2, 1) R⁷, —(C₁₋₅ alkylene)ₚOR⁸, and —C(═O)R⁹, wherein the —(C₁₋₅ alkylene) is optionally substituted with 1-5 halide (e.g., F, Cl, Br, I) and/or 1-3 (e.g., 1-2, 1) unsubstituted —(C₁₋₃ alkyl).

In some embodiments of Formula I, each R⁵ is independently selected from the group consisting of F, Cl, unsubstituted —(C₁₋₅ alkyl), unsubstituted —(C₂₋₅ alkenyl), unsubstituted —(C₂₋₅ alkynyl), unsubstituted —(C₁₋₅ haloalkyl), -heterocyclyl optionally substituted with 1-2 R⁷, —(C₁₋₂ alkylene)ₚOR⁸, and —C(═O)R⁹, wherein the —(C₁₋₂ alkylene) is optionally substituted with 1-2 halide (e.g., F, Cl).

In some embodiments of Formula I, each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁷, -carbocyclyl optionally substituted with 1-12 R¹⁴, —(C₁₋₅ alkylene)ₚOR⁸, and —C(═O)R⁹, wherein the —(C₁₋₅ alkylene) is optionally substituted with 1-5 halide and/or 1-3 unsubstituted —(C₁₋₃ alkyl).

In some embodiments of Formula I, each R⁵ is independently selected from the group consisting of F, Cl, unsubstituted —(C₁₋₅ alkyl), unsubstituted —(C₂₋₅ alkenyl), unsubstituted —(C₂₋₅ alkynyl), unsubstituted —(C₁₋₅ haloalkyl), -heterocyclyl optionally substituted with 1-2 R⁷, -carbocyclyl optionally substituted with 1-2 R¹⁴, —(C₁₋₂ alkylene)ₚOR⁸, and —C(═O)R⁹, wherein the —(C₁₋₂ alkylene) is optionally substituted with 1-2 halide (e.g., F, Cl).

In some embodiments of Formula I, two R attached to the same carbon atom are taken together to form a carbonyl group.

In some embodiments of Formula I, each R⁶ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁰, —C(═O)R¹¹, and —NHC(═O)R¹².

In some embodiments of Formula I, each R⁶ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), unsubstituted —(C₁₋₄ haloalkyl), —OR¹⁰, —C(═O)R¹¹, and —NHC(═O)R¹².

In some embodiments of Formula I, each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —(C₁₋₅ alkylene)ₚOR¹⁰, —C(═O)R¹¹, —NHC(═O)R¹², and -carbocyclyl optionally substituted with 1-12 (e.g., 1-11, 1-10, 1-9, 1-8, 1-7, 1-6, 1-5, 1-4, 1-3, 1-2, 1) R¹⁴, wherein the —(C₁₋₅ alkylene) is optionally substituted with 1-5 halide and/or 1-3 unsubstituted —(C₁₋₃ alkyl).

In some embodiments of Formula I, each R⁶ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), unsubstituted —(C₁₋₄ haloalkyl), —CN, —(C₁₋₂ alkylene)ₚOR¹⁰, —C(═O)R¹¹, —NHC(═O)R¹², and -carbocyclyl optionally substituted with 1-2 R¹⁴, wherein the —(C₁₋₂ alkylene) is optionally substituted with 1-2 halide (e.g., F, Cl) and/or 1 unsubstituted —(C₁₋₂ alkyl).

In some embodiments of Formula I, each R⁷ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R⁷ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, each R⁷ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, and -carbocyclyl optionally substituted with 1-12 R¹⁴.

In some embodiments of Formula I, each R⁷ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), unsubstituted —(C₁₋₄ haloalkyl), —CN, and -carbocyclyl optionally substituted with 1-2 R¹⁴.

In some embodiments of Formula I, each R⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R⁸ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, each R⁹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R⁹ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, each R¹⁰ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)ₚOR⁸.

In some embodiments of Formula I, each R¹⁰ is independently selected from the group consisting of H, unsubstituted —(C₁₋₄ alkyl), unsubstituted —(C₂₋₄ alkenyl), unsubstituted —(C₂₋₄ alkynyl), unsubstituted —(C₁₋₄ haloalkyl), and —(C₁₋₂ alkylene)ₚOR⁸.

In some embodiments of Formula I, each R¹¹ is —N(R¹³)₂.

In some embodiments of Formula I, each R¹¹ is —NHR¹³.

In some embodiments of Formula I, each R¹¹ is —NHMe.

In some embodiments of Formula I, each R¹¹ is —NHMe₂.

In some embodiments of Formula I, each R¹² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R¹² is independently selected from the group consisting of unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, each R¹³ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R¹³ is independently selected from the group consisting of unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, each R¹⁴ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R¹⁴ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, R¹⁵ is selected from the group consisting of H and halide (e.g., F, Cl, Br, I).

In some embodiments of Formula I, R¹⁵ is selected from the group consisting of H and halide (e.g., F, Cl).

In some embodiments of Formula I, R¹⁵ is H.

In some embodiments of Formula I, R¹⁵ is F.

In some embodiments of Formula I, R^{15a} is selected from the group consisting of H, halide (e.g., F, Cl, Br, I) and ²H (D) (deuterium).

In some embodiments of Formula I, R^{15b} is selected from the group consisting of H and ²H (D) (deuterium).

In some embodiments of Formula I, R^{15b} is ²H (D) (deuterium).

In some embodiments of Formula I, each R¹⁶ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R¹⁶ is independently selected from the group consisting of halide (e.g., F, Cl), unsubstituted —(C₁₋₄ alkyl), unsubstituted —(C₂₋₄ alkenyl), unsubstituted —(C₂₋₄ alkynyl), and unsubstituted —(C₁₋₄ haloalkyl).

In some embodiments of Formula I, each p is independently 0 or 1.

In some embodiments of Formula I, each H atom is optionally, independently replaced by ²H (D) (deuterium).

Illustrative compounds of Formula I are shown in Table 1.

TABLE 1

| | |
|---|---|
| 1 | 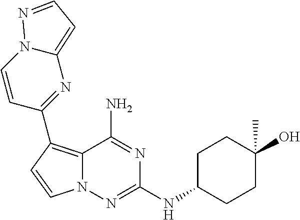 [SMILES: C[C@]1(O)CC[C@H](Nc2nc(N)c3c(-c4ccn5nccc5n4)ccn3n2)CC1] |
| 2 | 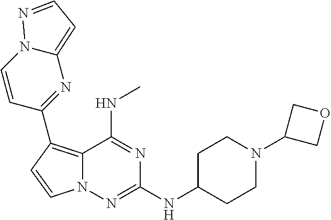 [SMILES: CNc1nc(NC2CCN(C3COC3)CC2)nn2ccc(-c3ccn4nccc4n3)c12] |
| 3 | 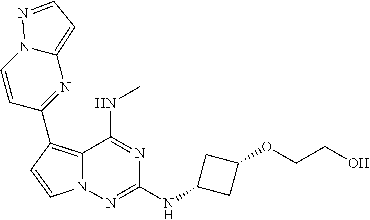 [SMILES: CNc1nc(N[C@H]2C[C@@H](OCCO)C2)nn2ccc(-c3ccn4nccc4n3)c12] |
| 4 | 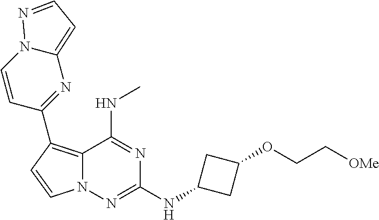 [SMILES: CNc1nc(N[C@H]2C[C@@H](OCCOC)C2)nn2ccc(-c3ccn4nccc4n3)c12] |
| 5 | 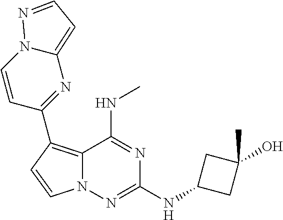 [SMILES: CNc1nc(N[C@H]2C[C@@](C)(O)C2)nn2ccc(-c3ccn4nccc4n3)c12] |
| 6 | 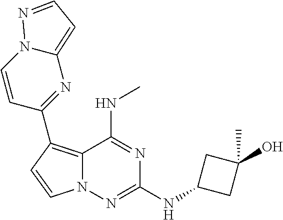 [SMILES: CNc1nc(N[C@H]2C[C@](C)(O)C2)nn2ccc(-c3ccn4nccc4n3)c12] |
| 7 | 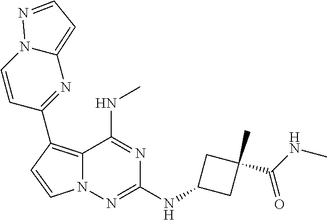 [SMILES: CNc1nc(N[C@H]2C[C@@](C)(C(=O)NC)C2)nn2ccc(-c3ccn4nccc4n3)c12] |
| 8 | 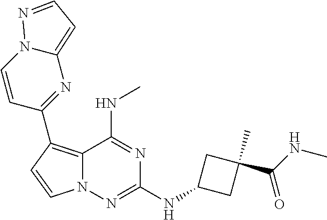 [SMILES: CNc1nc(N[C@H]2C[C@](C)(C(=O)NC)C2)nn2ccc(-c3ccn4nccc4n3)c12] |
| 9 | 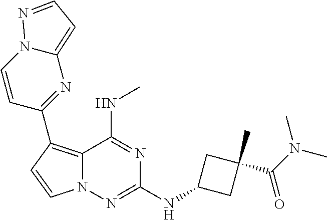 [SMILES: CNc1nc(N[C@H]2C[C@@](C)(C(=O)N(C)C)C2)nn2ccc(-c3ccn4nccc4n3)c12] |
| 10 | 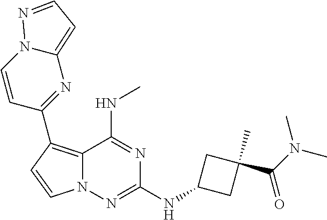 [SMILES: CNc1nc(N[C@H]2C[C@](C)(C(=O)N(C)C)C2)nn2ccc(-c3ccn4nccc4n3)c12] |
| 11 | 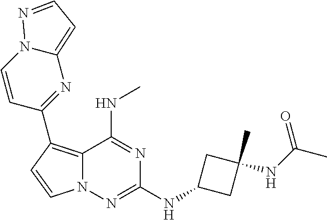 [SMILES: CNc1nc(N[C@H]2C[C@@](C)(NC(C)=O)C2)nn2ccc(-c3ccn4nccc4n3)c12] |
| 12 | 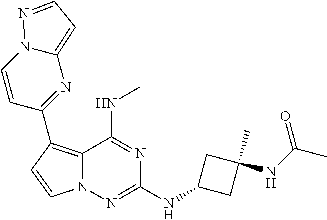 [SMILES: CNc1nc(N[C@H]2C[C@](C)(NC(C)=O)C2)nn2ccc(-c3ccn4nccc4n3)c12] |
| 13 | 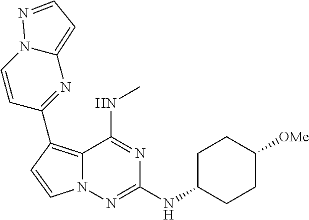 [SMILES: CNc1nc(N[C@H]2CC[C@@H](OC)CC2)nn2ccc(-c3ccn4nccc4n3)c12] |
| 14 | 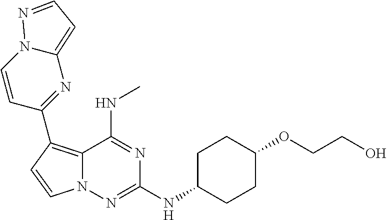 [SMILES: CNc1nc(N[C@H]2CC[C@@H](OCCO)CC2)nn2ccc(-c3ccn4nccc4n3)c12] |
| 15 | 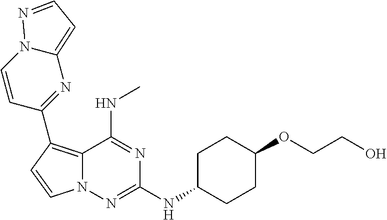 [SMILES: CNc1nc(N[C@H]2CC[C@H](OCCO)CC2)nn2ccc(-c3ccn4nccc4n3)c12] |
| 16 | 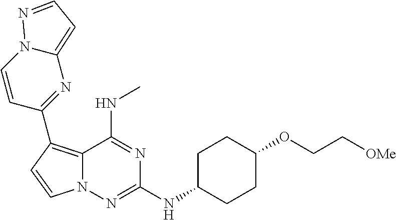 [SMILES: CNc1nc(N[C@H]2CC[C@@H](OCCOC)CC2)nn2ccc(-c3ccn4nccc4n3)c12] |
| 17 | 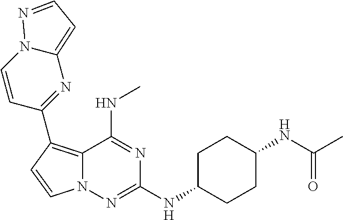 [SMILES: CNc1nc(N[C@H]2CC[C@@H](NC(C)=O)CC2)nn2ccc(-c3ccn4nccc4n3)c12] |
| 18 | 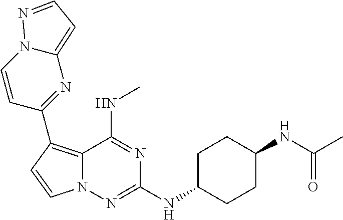 [SMILES: CNc1nc(N[C@H]2CC[C@H](NC(C)=O)CC2)nn2ccc(-c3ccn4nccc4n3)c12] |
| 19 | 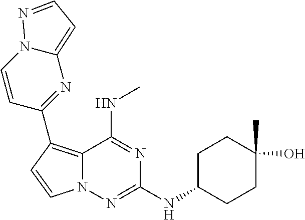 [SMILES: CNc1nc(N[C@H]2CC[C@@](C)(O)CC2)nn2ccc(-c3ccn4nccc4n3)c12] |
| 20 | 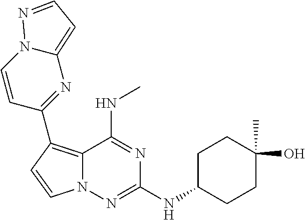 [SMILES: CNc1nc(N[C@H]2CC[C@](C)(O)CC2)nn2ccc(-c3ccn4nccc4n3)c12] |
| 21 | 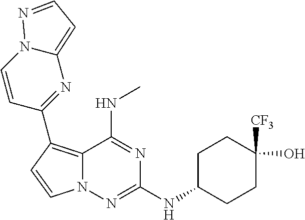 [SMILES: CNc1nc(N[C@H]2CC[C@](O)(C(F)(F)F)CC2)nn2ccc(-c3ccn4nccc4n3)c12] |
| 22 | 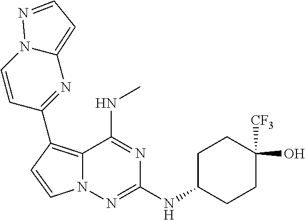 [SMILES: CNc1nc(N[C@H]2CC[C@@](O)(C(F)(F)F)CC2)nn2ccc(-c3ccn4nccc4n3)c12] |
| 23 | 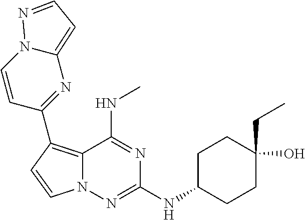 [SMILES: CC[C@]1(O)CC[C@@H](Nc2nc(NC)c3c(-c4ccn5nccc5n4)ccn3n2)CC1] |
| 24 | 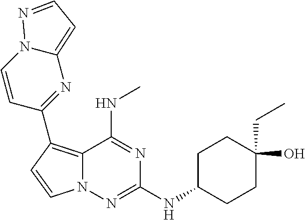 [SMILES: CC[C@]1(O)CC[C@H](Nc2nc(NC)c3c(-c4ccn5nccc5n4)ccn3n2)CC1] |
| 25 | 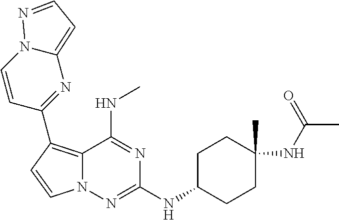 [SMILES: CNc1nc(N[C@H]2CC[C@@](C)(NC(C)=O)CC2)nn2ccc(-c3ccn4nccc4n3)c12] |
| 26 | 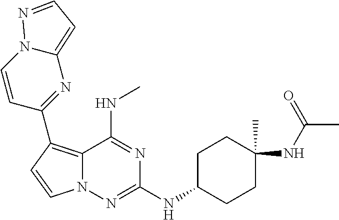 [SMILES: CNc1nc(N[C@H]2CC[C@](C)(NC(C)=O)CC2)nn2ccc(-c3ccn4nccc4n3)c12] |
| 27 | 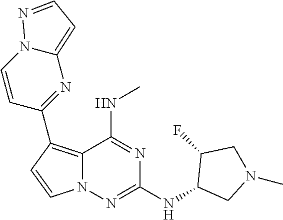 [SMILES: CNc1nc(N[C@H]2CN(C)C[C@H]2F)nn2ccc(-c3ccn4nccc4n3)c12] |
| 28 | 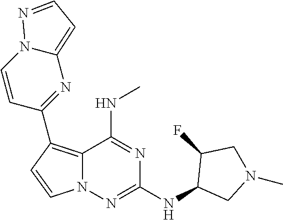 [SMILES: CNc1nc(N[C@@H]2CN(C)C[C@@H]2F)nn2ccc(-c3ccn4nccc4n3)c12] |
| 29 | 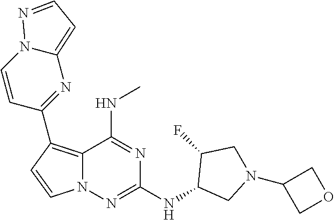 [SMILES: CNc1nc(N[C@H]2CN(C3COC3)C[C@H]2F)nn2ccc(-c3ccn4nccc4n3)c12] |
| 30 | 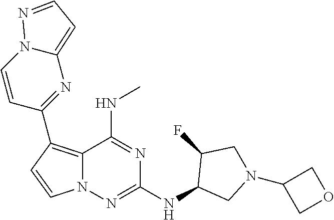 [SMILES: CNc1nc(N[C@@H]2CN(C3COC3)C[C@@H]2F)nn2ccc(-c3ccn4nccc4n3)c12] |
| 31 | 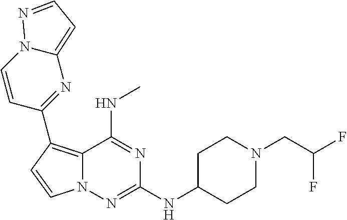 [SMILES: CNc1nc(NC2CCN(CC(F)F)CC2)nn2ccc(-c3ccn4nccc4n3)c12] |
| 32 | 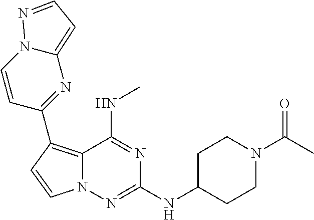 [SMILES: CNc1nc(NC2CCN(C(C)=O)CC2)nn2ccc(-c3ccn4nccc4n3)c12] |
| 33 | 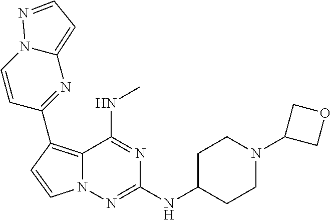 [SMILES: CNc1nc(NC2CCN(C3COC3)CC2)nn2ccc(-c3ccn4nccc4n3)c12] |
| 34 | 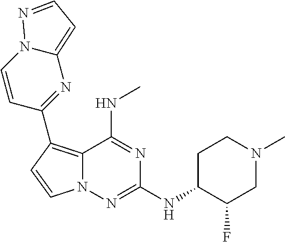 [SMILES: CNc1nc(N[C@@H]2CCN(C)C[C@@H]2F)nn2ccc(-c3ccn4nccc4n3)c12] |
| 35 | 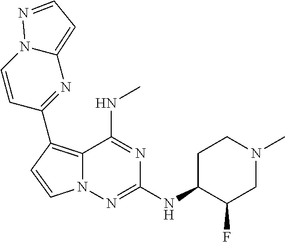 [SMILES: CNc1nc(N[C@H]2CCN(C)C[C@H]2F)nn2ccc(-c3ccn4nccc4n3)c12] |
| 36 | 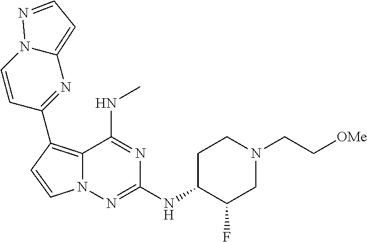 [SMILES: CNc1nc(N[C@@H]2CCN(CCOC)C[C@@H]2F)nn2ccc(-c3ccn4nccc4n3)c12] |
| 37 | 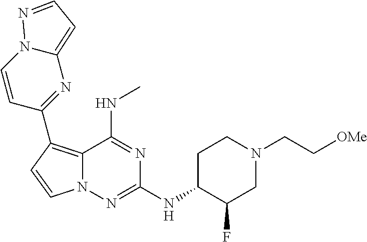 [SMILES: CNc1nc(N[C@@H]2CCN(CCOC)C[C@H]2F)nn2ccc(-c3ccn4nccc4n3)c12] |
| 38 | 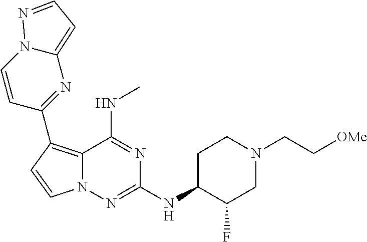 [SMILES: CNc1nc(N[C@H]2CCN(CCOC)C[C@@H]2F)nn2ccc(-c3ccn4nccc4n3)c12] |
| 39 | 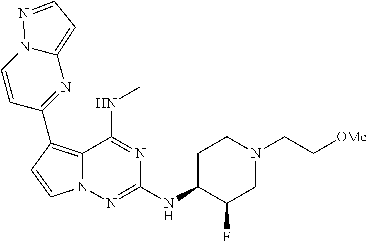 [SMILES: CNc1nc(N[C@H]2CCN(CCOC)C[C@H]2F)nn2ccc(-c3ccn4nccc4n3)c12] |
| 40 | 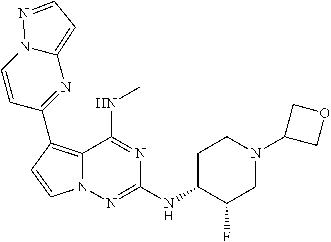 [SMILES: CNc1nc(N[C@@H]2CCN(C3COC3)C[C@@H]2F)nn2ccc(-c3ccn4nccc4n3)c12] |
| 41 | 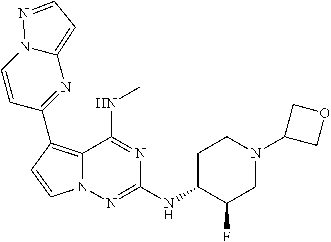 [SMILES: CNc1nc(N[C@@H]2CCN(C3COC3)C[C@H]2F)nn2ccc(-c3ccn4nccc4n3)c12] |
| 42 | 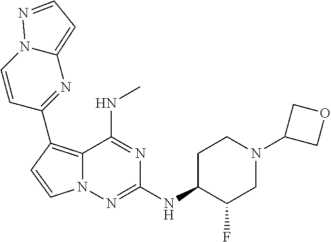 [SMILES: CNc1nc(N[C@H]2CCN(C3COC3)C[C@@H]2F)nn2ccc(-c3ccn4nccc4n3)c12] |
| 43 | 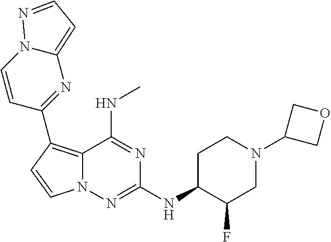 [SMILES: CNc1nc(N[C@H]2CCN(C3COC3)C[C@H]2F)nn2ccc(-c3ccn4nccc4n3)c12] |
| 44 | 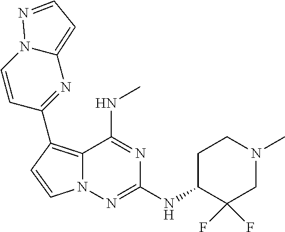 [SMILES: CNc1nc(N[C@@H]2CCN(C)CC2(F)F)nn2ccc(-c3ccn4nccc4n3)c12] |
| 45 | 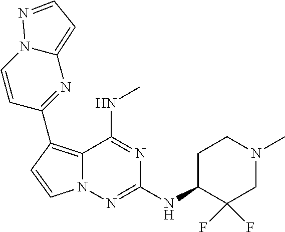 [SMILES: CNc1nc(N[C@H]2CCN(C)CC2(F)F)nn2ccc(-c3ccn4nccc4n3)c12] |
| 46 | 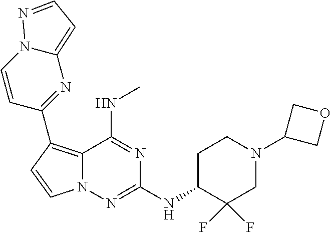 [SMILES: CNc1nc(N[C@@H]2CCN(C3COC3)CC2(F)F)nn2ccc(-c3ccn4nccc4n3)c12] |
| 47 | 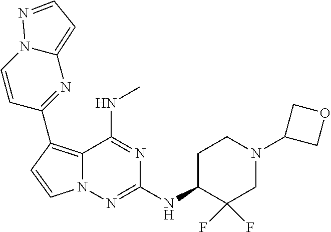 [SMILES: CNc1nc(N[C@H]2CCN(C3COC3)CC2(F)F)nn2ccc(-c3ccn4nccc4n3)c12] |
| 48 | 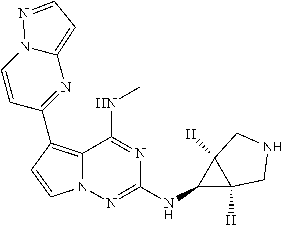 [SMILES: CNc1nc(N[C@@H]2[C@@H]3CNC[C@@H]32)nn2ccc(-c3ccn4nccc4n3)c12] |
| 49 | 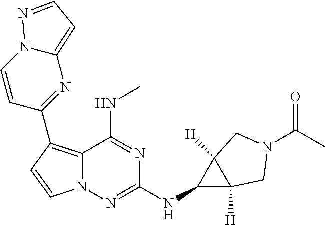 [SMILES: CNc1nc(N[C@@H]2[C@@H]3CN(C(C)=O)C[C@@H]32)nn2ccc(-c3ccn4nccc4n3)c12] |
| 50 | 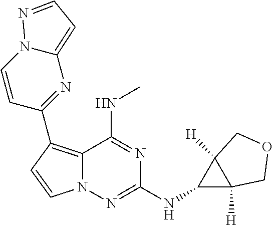 [SMILES: CNc1nc(N[C@H]2[C@@H]3COC[C@@H]32)nn2ccc(-c3ccn4nccc4n3)c12] |
| 51 | 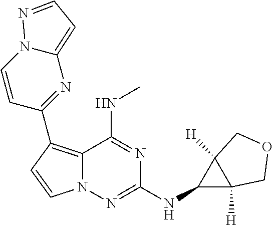 [SMILES: CNc1nc(N[C@@H]2[C@@H]3COC[C@@H]32)nn2ccc(-c3ccn4nccc4n3)c12] |
| 52 | 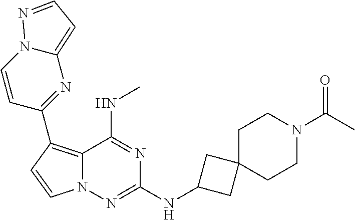 [SMILES: CNc1nc(NC2CC3(CCN(C(C)=O)CC3)C2)nn2ccc(-c3ccn4nccc4n3)c12] |
| 53 | 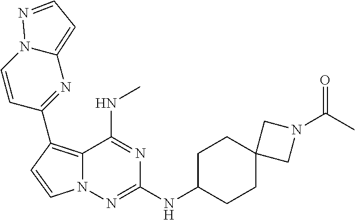 [SMILES: CNc1nc(NC2CCC3(CC2)CN(C(C)=O)C3)nn2ccc(-c3ccn4nccc4n3)c12] |
| 54 | 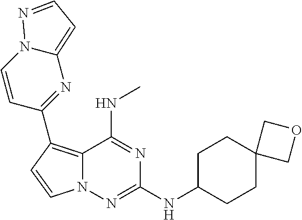 [SMILES: CNc1nc(NC2CCC3(CC2)COC3)nn2ccc(-c3ccn4nccc4n3)c12] |
| 55 | 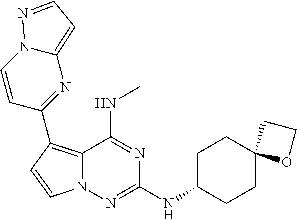 [SMILES: CNc1nc(N[C@H]2CC[C@@]3(CCO3)CC2)nn2ccc(-c3ccn4nccc4n3)c12] |
| 56 | 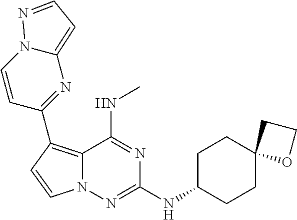 [SMILES: CNc1nc(N[C@H]2CC[C@]3(CCO3)CC2)nn2ccc(-c3ccn4nccc4n3)c12] |
| 57 | 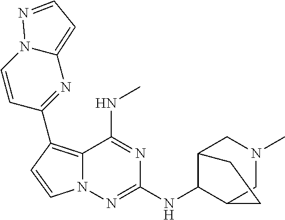 [SMILES: CNc1nc(NC2C3CCC2CN(C)C3)nn2ccc(-c3ccn4nccc4n3)c12] |
| 58 | 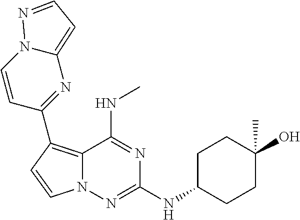 [SMILES: CNc1nc(N[C@H]2CC[C@](C)(O)CC2)nn2ccc(-c3ccn4nccc4n3)c12] |
| 59 | 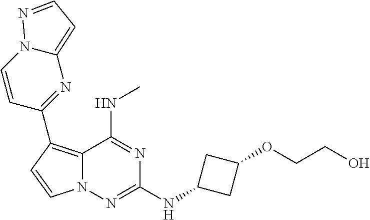 [SMILES: CNc1nc(N[C@H]2C[C@@H](OCCO)C2)nn2ccc(-c3ccn4nccc4n3)c12] |
| 60 | 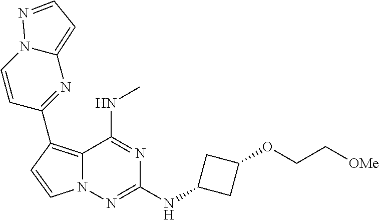 [SMILES: CNc1nc(N[C@H]2C[C@@H](OCCOC)C2)nn2ccc(-c3ccn4nccc4n3)c12] |
| 61 | 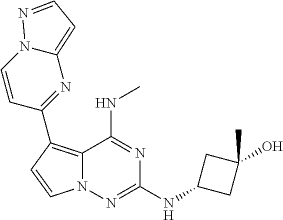 [SMILES: CNc1nc(N[C@H]2C[C@@](C)(O)C2)nn2ccc(-c3ccn4nccc4n3)c12] |
| 62 | 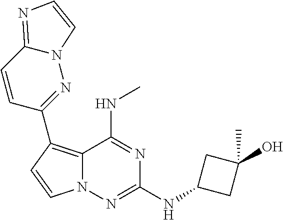 [SMILES: CNc1nc(N[C@H]2C[C@](C)(O)C2)nn2ccc(-c3ccc4nccn4n3)c12] |
| 63 | 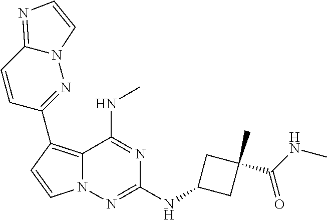 [SMILES: CNc1nc(N[C@H]2C[C@@](C)(C(=O)NC)C2)nn2ccc(-c3ccc4nccn4n3)c12] |
| 64 | 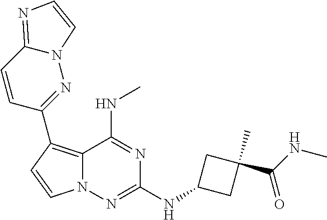 [SMILES: CNc1nc(N[C@H]2C[C@](C)(C(=O)NC)C2)nn2ccc(-c3ccc4nccn4n3)c12] |
| 65 | 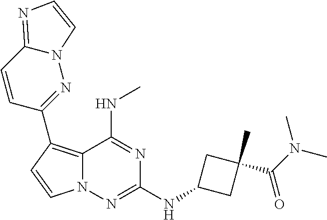 [SMILES: CNc1nc(N[C@H]2C[C@@](C)(C(=O)N(C)C)C2)nn2ccc(-c3ccc4nccn4n3)c12] |
| 66 | 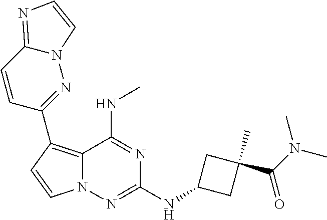 [SMILES: CNc1nc(N[C@H]2C[C@](C)(C(=O)N(C)C)C2)nn2ccc(-c3ccc4nccn4n3)c12] |
| 67 | 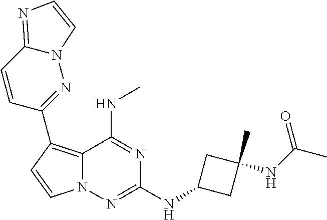 [SMILES: CNc1nc(N[C@H]2C[C@@](C)(NC(C)=O)C2)nn2ccc(-c3ccc4nccn4n3)c12] |
| 68 | 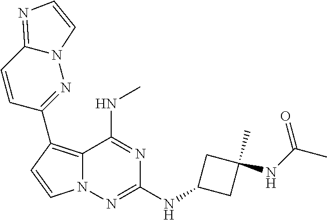 [SMILES: CNc1nc(N[C@H]2C[C@](C)(NC(C)=O)C2)nn2ccc(-c3ccc4nccn4n3)c12] |
| 69 | 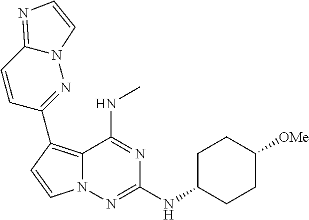 [SMILES: CNc1nc(N[C@H]2CC[C@@H](OC)CC2)nn2ccc(-c3ccc4nccn4n3)c12] |
| 70 | 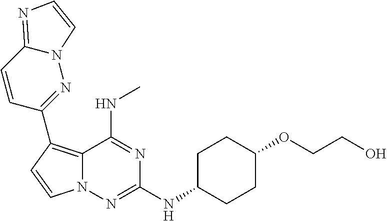 [SMILES: CNc1nc(N[C@H]2CC[C@@H](OCCO)CC2)nn2ccc(-c3ccc4nccn4n3)c12] |
| 71 | 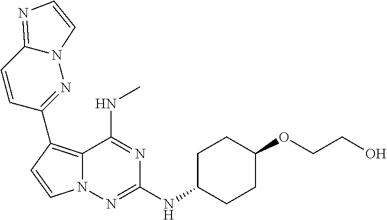 [SMILES: CNc1nc(N[C@H]2CC[C@H](OCCO)CC2)nn2ccc(-c3ccc4nccn4n3)c12] |
| 72 | 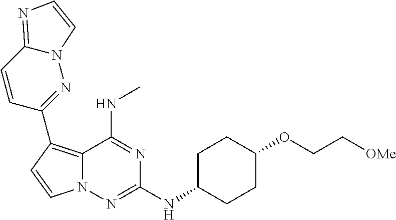 [SMILES: CNc1nc(N[C@H]2CC[C@@H](OCCOC)CC2)nn2ccc(-c3ccc4nccn4n3)c12] |
| 73 | 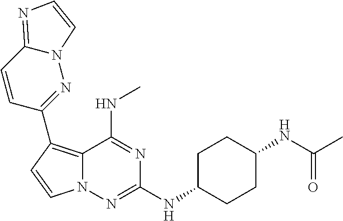 [SMILES: CNc1nc(N[C@H]2CC[C@@H](NC(C)=O)CC2)nn2ccc(-c3ccc4nccn4n3)c12] |
| 74 | 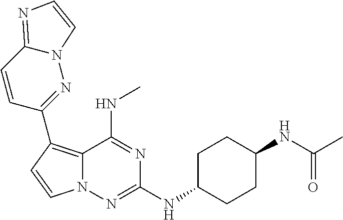 [SMILES: CNc1nc(N[C@H]2CC[C@H](NC(C)=O)CC2)nn2ccc(-c3ccc4nccn4n3)c12] |
| 75 | 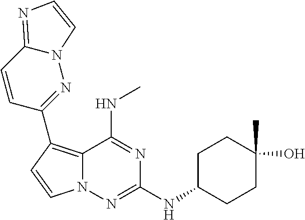 [SMILES: CNc1nc(N[C@H]2CC[C@@](C)(O)CC2)nn2ccc(-c3ccc4nccn4n3)c12] |
| 76 | 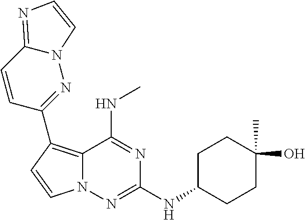 [SMILES: CNc1nc(N[C@H]2CC[C@](C)(O)CC2)nn2ccc(-c3ccc4nccn4n3)c12] |
| 77 | 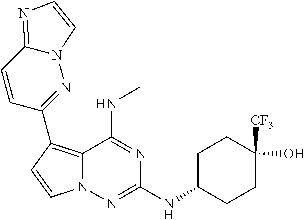 [SMILES: CNc1nc(N[C@H]2CC[C@](O)(C(F)(F)F)CC2)nn2ccc(-c3ccc4nccn4n3)c12] |
| 78 | 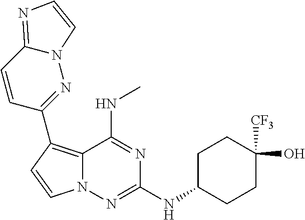 [SMILES: CNc1nc(N[C@H]2CC[C@@](O)(C(F)(F)F)CC2)nn2ccc(-c3ccc4nccn4n3)c12] |
| 79 | 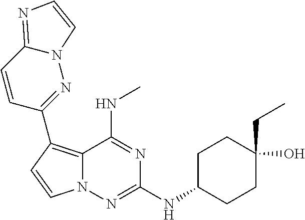 [SMILES: CC[C@]1(O)CC[C@@H](Nc2nc(NC)c3c(-c4ccc5nccn5n4)ccn3n2)CC1] |
| 80 | 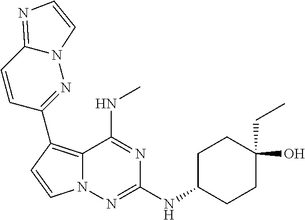 [SMILES: CC[C@]1(O)CC[C@H](Nc2nc(NC)c3c(-c4ccc5nccn5n4)ccn3n2)CC1] |
| 81 | 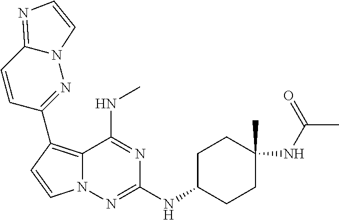 [SMILES: CNc1nc(N[C@H]2CC[C@@](C)(NC(C)=O)CC2)nn2ccc(-c3ccc4nccn4n3)c12] |
| 82 | 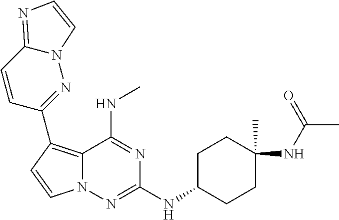 [SMILES: CNc1nc(N[C@H]2CC[C@](C)(NC(C)=O)CC2)nn2ccc(-c3ccc4nccn4n3)c12] |
| 83 | 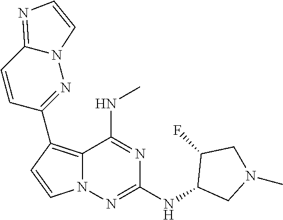 [SMILES: CNc1nc(N[C@H]2CN(C)C[C@H]2F)nn2ccc(-c3ccc4nccn4n3)c12] |
| 84 | 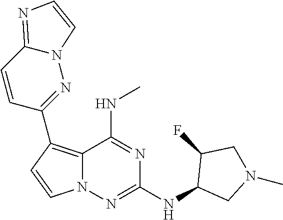 [SMILES: CNc1nc(N[C@@H]2CN(C)C[C@@H]2F)nn2ccc(-c3ccc4nccn4n3)c12] |
| 85 | 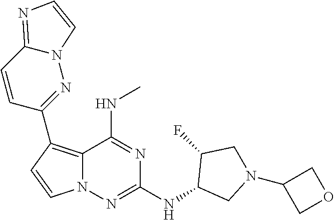 [SMILES: CNc1nc(N[C@H]2CN(C3COC3)C[C@H]2F)nn2ccc(-c3ccc4nccn4n3)c12] |
| 86 | 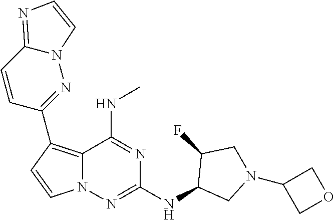 [SMILES: CNc1nc(N[C@@H]2CN(C3COC3)C[C@@H]2F)nn2ccc(-c3ccc4nccn4n3)c12] |
| 87 | 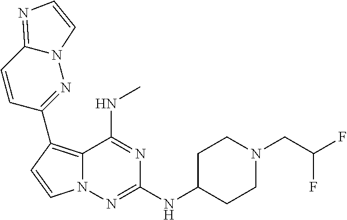 [SMILES: CNc1nc(NC2CCN(CC(F)F)CC2)nn2ccc(-c3ccc4nccn4n3)c12] |
| 88 | 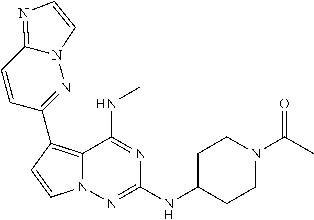 [SMILES: CNc1nc(NC2CCN(C(C)=O)CC2)nn2ccc(-c3ccc4nccn4n3)c12] |
| 89 | 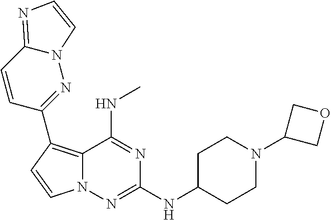 [SMILES: CNc1nc(NC2CCN(C3COC3)CC2)nn2ccc(-c3ccc4nccn4n3)c12] |
| 90 | 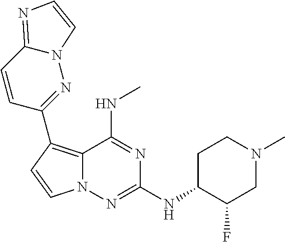 [SMILES: CNc1nc(N[C@@H]2CCN(C)C[C@@H]2F)nn2ccc(-c3ccc4nccn4n3)c12] |
| 91 | 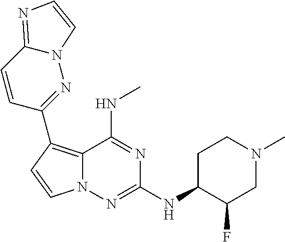 [SMILES: CNc1nc(N[C@H]2CCN(C)C[C@H]2F)nn2ccc(-c3ccc4nccn4n3)c12] |
| 92 | 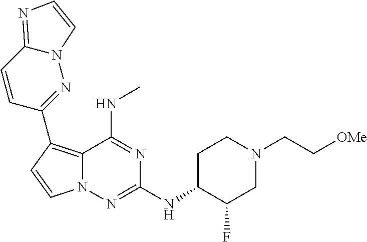 [SMILES: CNc1nc(N[C@@H]2CCN(CCOC)C[C@@H]2F)nn2ccc(-c3ccc4nccn4n3)c12] |
| 93 | 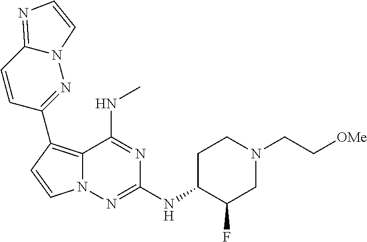 [SMILES: CNc1nc(N[C@@H]2CCN(CCOC)C[C@H]2F)nn2ccc(-c3ccc4nccn4n3)c12] |
| 94 | 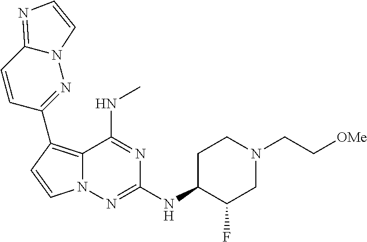 [SMILES: CNc1nc(N[C@H]2CCN(CCOC)C[C@@H]2F)nn2ccc(-c3ccc4nccn4n3)c12] |
| 95 | 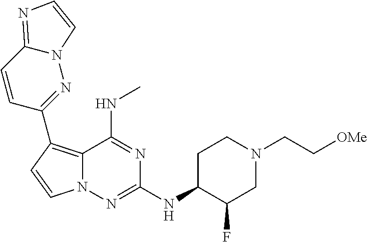 [SMILES: CNc1nc(N[C@H]2CCN(CCOC)C[C@H]2F)nn2ccc(-c3ccc4nccn4n3)c12] |
| 96 | 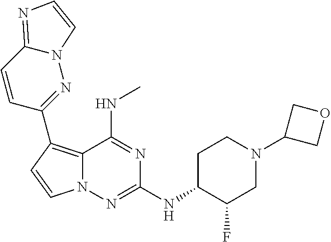 [SMILES: CNc1nc(N[C@@H]2CCN(C3COC3)C[C@@H]2F)nn2ccc(-c3ccc4nccn4n3)c12] |
| 97 | 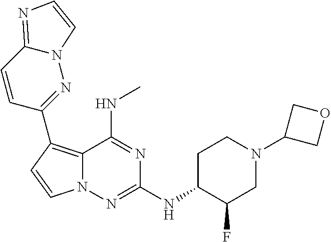 [SMILES: CNc1nc(N[C@@H]2CCN(C3COC3)C[C@H]2F)nn2ccc(-c3ccc4nccn4n3)c12] |
| 98 | 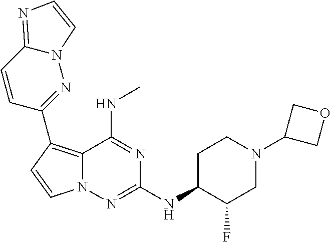 [SMILES: CNc1nc(N[C@H]2CCN(C3COC3)C[C@@H]2F)nn2ccc(-c3ccc4nccn4n3)c12] |
| 99 | 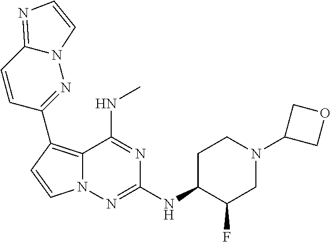 [SMILES: CNc1nc(N[C@H]2CCN(C3COC3)C[C@H]2F)nn2ccc(-c3ccc4nccn4n3)c12] |
| 100 | 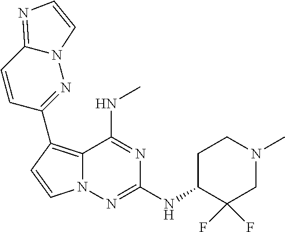 [SMILES: CNc1nc(N[C@@H]2CCN(C)CC2(F)F)nn2ccc(-c3ccc4nccn4n3)c12] |
| 101 | 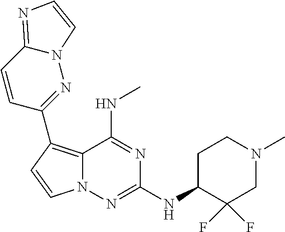 [SMILES: CNc1nc(N[C@H]2CCN(C)CC2(F)F)nn2ccc(-c3ccc4nccn4n3)c12] |
| 102 | 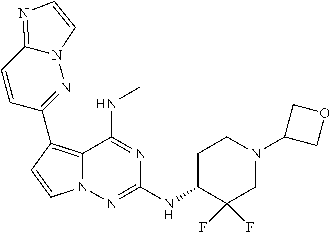 [SMILES: CNc1nc(N[C@@H]2CCN(C3COC3)CC2(F)F)nn2ccc(-c3ccc4nccn4n3)c12] |
| 103 | 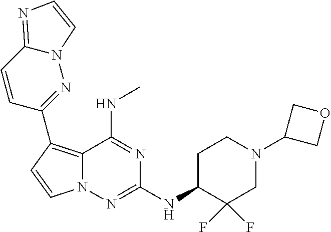 [SMILES: CNc1nc(N[C@H]2CCN(C3COC3)CC2(F)F)nn2ccc(-c3ccc4nccn4n3)c12] |
| 104 | 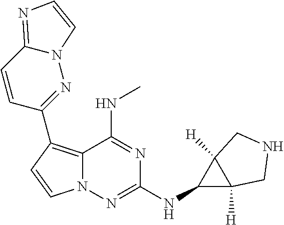 [SMILES: CNc1nc(N[C@@H]2[C@@H]3CNC[C@@H]32)nn2ccc(-c3ccc4nccn4n3)c12] |
| 105 | 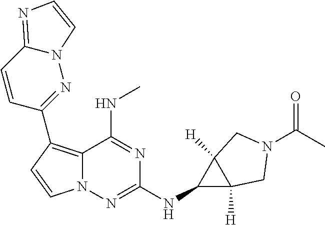 [SMILES: CNc1nc(N[C@@H]2[C@@H]3CN(C(C)=O)C[C@@H]32)nn2ccc(-c3ccc4nccn4n3)c12] |
| 106 | 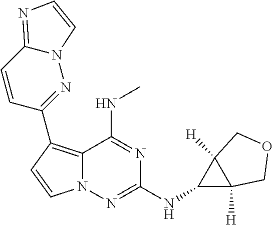 [SMILES: CNc1nc(N[C@H]2[C@@H]3COC[C@@H]32)nn2ccc(-c3ccc4nccn4n3)c12] |
| 107 | 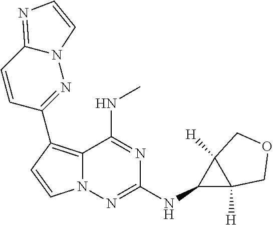 [SMILES: CNc1nc(N[C@@H]2[C@@H]3COC[C@@H]32)nn2ccc(-c3ccc4nccn4n3)c12] |
| 108 | 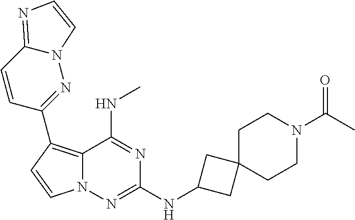 [SMILES: CNc1nc(NC2CC3(CCN(C(C)=O)CC3)C2)nn2ccc(-c3ccc4nccn4n3)c12] |
| 109 | 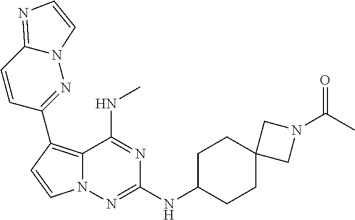 [SMILES: CNc1nc(NC2CCC3(CC2)CN(C(C)=O)C3)nn2ccc(-c3ccc4nccn4n3)c12] |
| 110 | 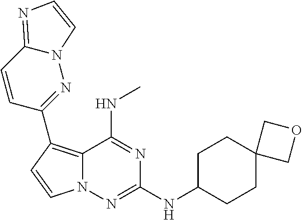 [SMILES: CNc1nc(NC2CCC3(CC2)COC3)nn2ccc(-c3ccc4nccn4n3)c12] |
| 111 | 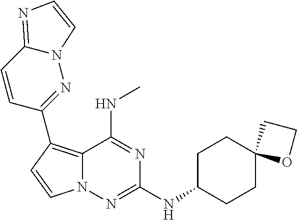 [SMILES: CNc1nc(N[C@H]2CC[C@@]3(CCO3)CC2)nn2ccc(-c3ccc4nccn4n3)c12] |
| 112 | 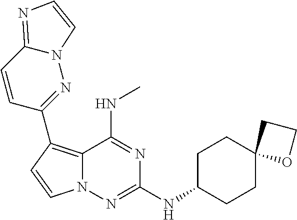 [SMILES: CNc1nc(N[C@H]2CC[C@]3(CCO3)CC2)nn2ccc(-c3ccc4nccn4n3)c12] |
| 113 | 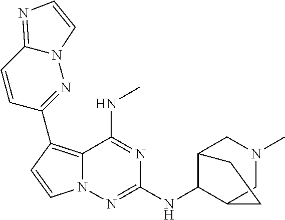 [SMILES: CNc1nc(NC2C3CCC2CN(C)C3)nn2ccc(-c3ccc4nccn4n3)c12] |
| 114 | 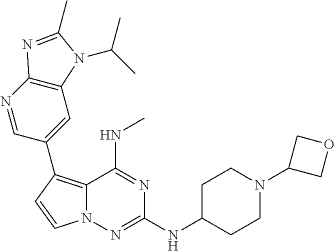 [SMILES: CNc1nc(NC2CCN(C3COC3)CC2)nn2ccc(-c3cnc4nc(C)n(C(C)C)c4c3)c12] |
| 115 | 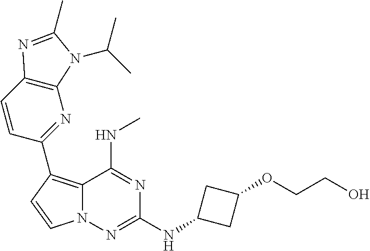 [SMILES: CNc1nc(N[C@H]2C[C@@H](OCCO)C2)nn2ccc(-c3ccc4nc(C)n(C(C)C)c4n3)c12] |
| 116 | 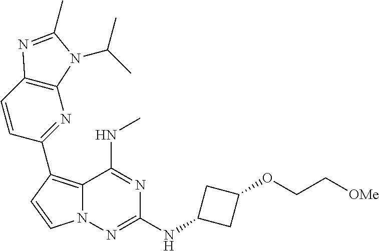 [SMILES: CNc1nc(N[C@H]2C[C@@H](OCCOC)C2)nn2ccc(-c3ccc4nc(C)n(C(C)C)c4n3)c12] |
| 117 | 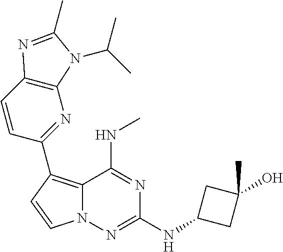 [SMILES: CNc1nc(N[C@H]2C[C@@](C)(O)C2)nn2ccc(-c3ccc4nc(C)n(C(C)C)c4n3)c12] |
| 118 | 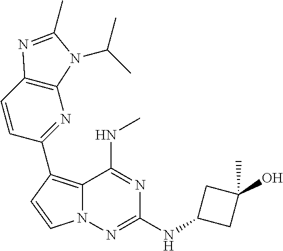 [SMILES: CNc1nc(N[C@H]2C[C@](C)(O)C2)nn2ccc(-c3ccc4nc(C)n(C(C)C)c4n3)c12] |
| 119 | 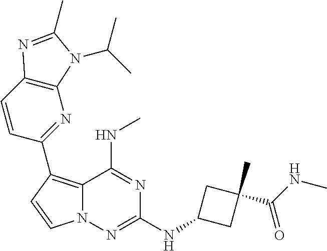 [SMILES: CNc1nc(N[C@H]2C[C@@](C)(C(=O)NC)C2)nn2ccc(-c3ccc4nc(C)n(C(C)C)c4n3)c12] |
| 120 | 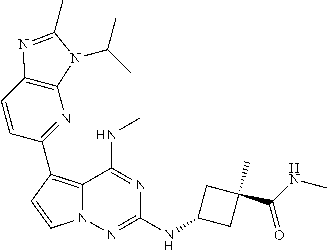 [SMILES: CNc1nc(N[C@H]2C[C@](C)(C(=O)NC)C2)nn2ccc(-c3ccc4nc(C)n(C(C)C)c4n3)c12] |
| 121 | 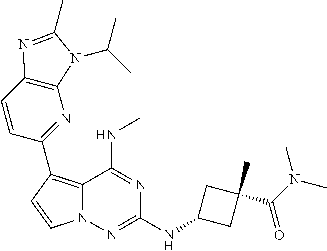 [SMILES: CNc1nc(N[C@H]2C[C@@](C)(C(=O)N(C)C)C2)nn2ccc(-c3ccc4nc(C)n(C(C)C)c4n3)c12] |
| 122 | 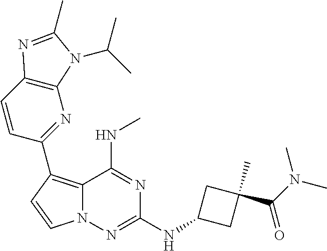 [SMILES: CNc1nc(N[C@H]2C[C@](C)(C(=O)N(C)C)C2)nn2ccc(-c3ccc4nc(C)n(C(C)C)c4n3)c12] |
| 123 | 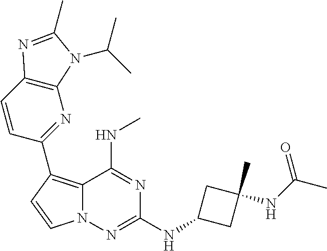 [SMILES: CNc1nc(N[C@H]2C[C@@](C)(NC(C)=O)C2)nn2ccc(-c3ccc4nc(C)n(C(C)C)c4n3)c12] |
| 124 | 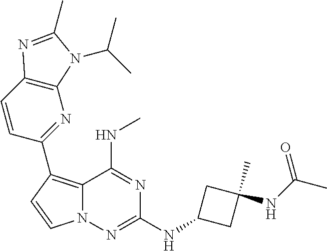 [SMILES: CNc1nc(N[C@H]2C[C@](C)(NC(C)=O)C2)nn2ccc(-c3ccc4nc(C)n(C(C)C)c4n3)c12] |
| 125 | 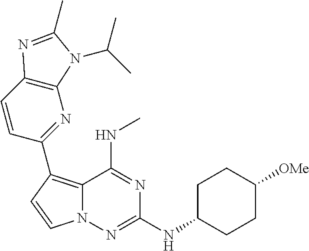 [SMILES: CNc1nc(N[C@H]2CC[C@@H](OC)CC2)nn2ccc(-c3ccc4nc(C)n(C(C)C)c4n3)c12] |
| 126 | 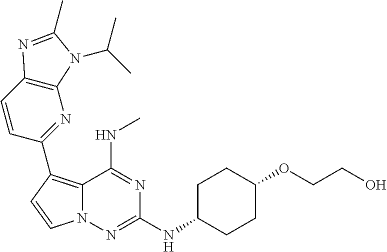 [SMILES: CNc1nc(N[C@H]2CC[C@@H](OCCO)CC2)nn2ccc(-c3ccc4nc(C)n(C(C)C)c4n3)c12] |
| 127 | 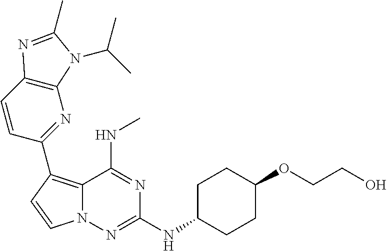 [SMILES: CNc1nc(N[C@H]2CC[C@H](OCCO)CC2)nn2ccc(-c3ccc4nc(C)n(C(C)C)c4n3)c12] |
| 128 | 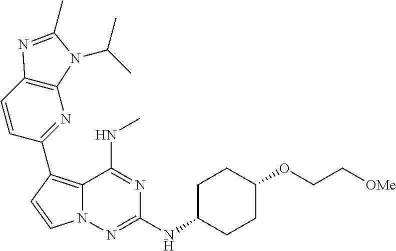 [SMILES: CNc1nc(N[C@H]2CC[C@@H](OCCOC)CC2)nn2ccc(-c3ccc4nc(C)n(C(C)C)c4n3)c12] |
| 129 | 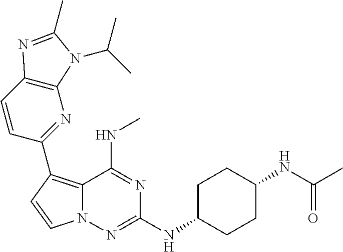 [SMILES: CNc1nc(N[C@H]2CC[C@@H](NC(C)=O)CC2)nn2ccc(-c3ccc4nc(C)n(C(C)C)c4n3)c12] |
| 130 | 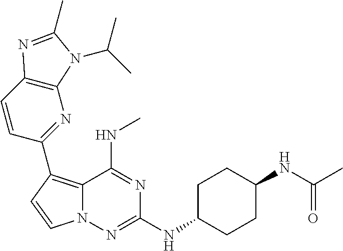 [SMILES: CNc1nc(N[C@H]2CC[C@H](NC(C)=O)CC2)nn2ccc(-c3ccc4nc(C)n(C(C)C)c4n3)c12] |
| 131 | 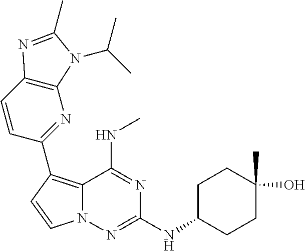 [SMILES: CNc1nc(N[C@H]2CC[C@@](C)(O)CC2)nn2ccc(-c3ccc4nc(C)n(C(C)C)c4n3)c12] |
| 132 | 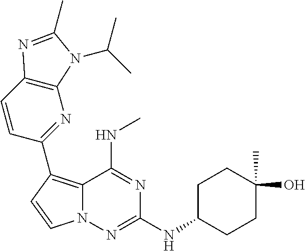 [SMILES: CNc1nc(N[C@H]2CC[C@](C)(O)CC2)nn2ccc(-c3ccc4nc(C)n(C(C)C)c4n3)c12] |
| 133 | 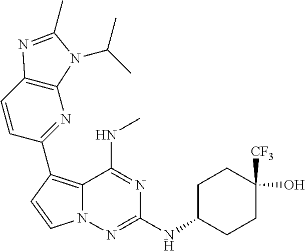 [SMILES: CNc1nc(N[C@H]2CC[C@](O)(C(F)(F)F)CC2)nn2ccc(-c3ccc4nc(C)n(C(C)C)c4n3)c12] |
| 134 | 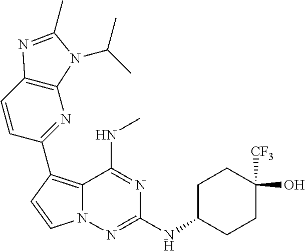 [SMILES: CNc1nc(N[C@H]2CC[C@@](O)(C(F)(F)F)CC2)nn2ccc(-c3ccc4nc(C)n(C(C)C)c4n3)c12] |
| 135 | 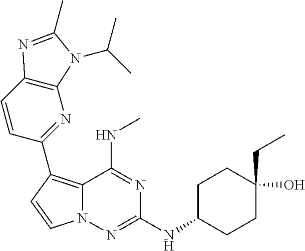 [SMILES: CC[C@]1(O)CC[C@@H](Nc2nc(NC)c3c(-c4ccc5nc(C)n(C(C)C)c5n4)ccn3n2)CC1] |
| 136 | 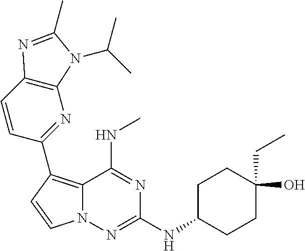 [SMILES: CC[C@]1(O)CC[C@H](Nc2nc(NC)c3c(-c4ccc5nc(C)n(C(C)C)c5n4)ccn3n2)CC1] |
| 137 | 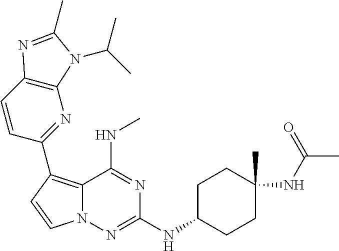 [SMILES: CNc1nc(N[C@H]2CC[C@@](C)(NC(C)=O)CC2)nn2ccc(-c3ccc4nc(C)n(C(C)C)c4n3)c12] |
| 138 | 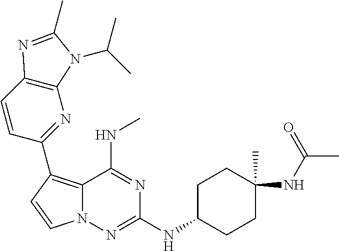 [SMILES: CNc1nc(N[C@H]2CC[C@](C)(NC(C)=O)CC2)nn2ccc(-c3ccc4nc(C)n(C(C)C)c4n3)c12] |
| 139 | 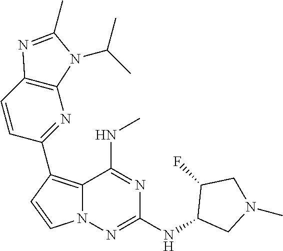 [SMILES: CNc1nc(N[C@H]2CN(C)C[C@H]2F)nn2ccc(-c3ccc4nc(C)n(C(C)C)c4n3)c12] |
| 140 | 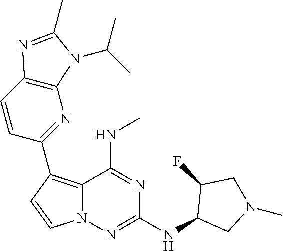 [SMILES: CNc1nc(N[C@@H]2CN(C)C[C@@H]2F)nn2ccc(-c3ccc4nc(C)n(C(C)C)c4n3)c12] |
| 141 | 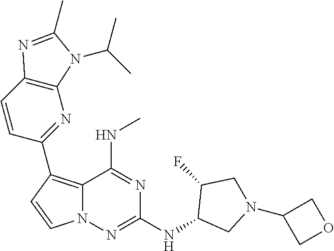 [SMILES: CNc1nc(N[C@H]2CN(C3COC3)C[C@H]2F)nn2ccc(-c3ccc4nc(C)n(C(C)C)c4n3)c12] |
| 142 | 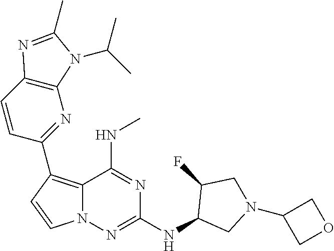 [SMILES: CNc1nc(N[C@@H]2CN(C3COC3)C[C@@H]2F)nn2ccc(-c3ccc4nc(C)n(C(C)C)c4n3)c12] |
| 143 | 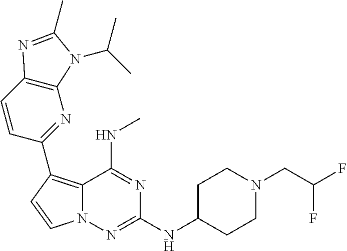 [SMILES: CNc1nc(NC2CCN(CC(F)F)CC2)nn2ccc(-c3ccc4nc(C)n(C(C)C)c4n3)c12] |
| 144 | 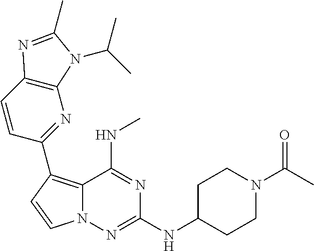 [SMILES: CNc1nc(NC2CCN(C(C)=O)CC2)nn2ccc(-c3ccc4nc(C)n(C(C)C)c4n3)c12] |
| 145 | 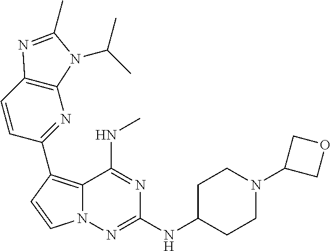 [SMILES: CNc1nc(NC2CCN(C3COC3)CC2)nn2ccc(-c3ccc4nc(C)n(C(C)C)c4n3)c12] |
| 146 | 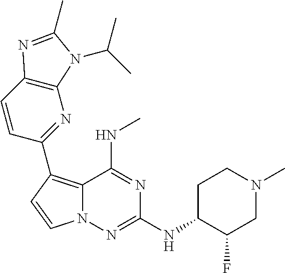 [SMILES: CNc1nc(N[C@@H]2CCN(C)C[C@@H]2F)nn2ccc(-c3ccc4nc(C)n(C(C)C)c4n3)c12] |
| 147 | 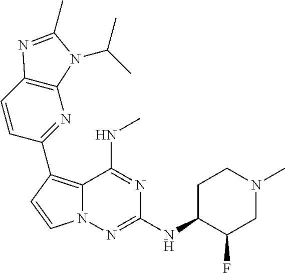 [SMILES: CNc1nc(N[C@H]2CCN(C)C[C@H]2F)nn2ccc(-c3ccc4nc(C)n(C(C)C)c4n3)c12] |
| 148 | 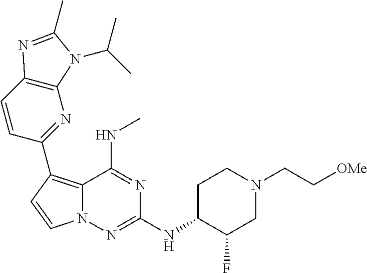 [SMILES: CNc1nc(N[C@@H]2CCN(CCOC)C[C@@H]2F)nn2ccc(-c3ccc4nc(C)n(C(C)C)c4n3)c12] |
| 149 | 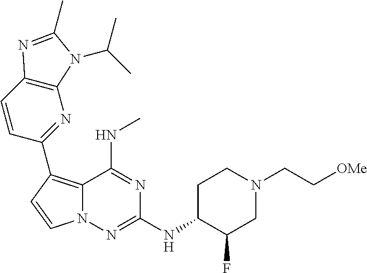 [SMILES: CNc1nc(N[C@@H]2CCN(CCOC)C[C@H]2F)nn2ccc(-c3ccc4nc(C)n(C(C)C)c4n3)c12] |
| 150 | 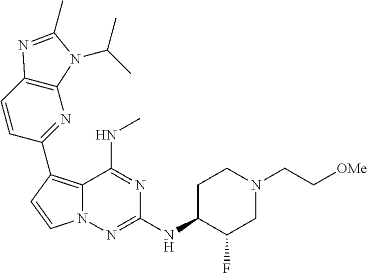 [SMILES: CNc1nc(N[C@H]2CCN(CCOC)C[C@@H]2F)nn2ccc(-c3ccc4nc(C)n(C(C)C)c4n3)c12] |
| 151 | 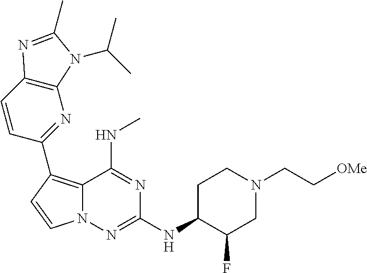 [SMILES: CNc1nc(N[C@H]2CCN(CCOC)C[C@H]2F)nn2ccc(-c3ccc4nc(C)n(C(C)C)c4n3)c12] |
| 152 | 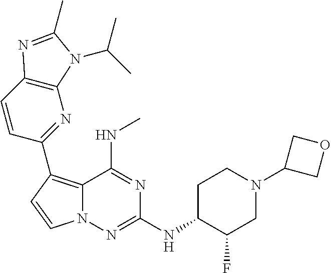 [SMILES: CNc1nc(N[C@@H]2CCN(C3COC3)C[C@@H]2F)nn2ccc(-c3ccc4nc(C)n(C(C)C)c4n3)c12] |
| 153 | 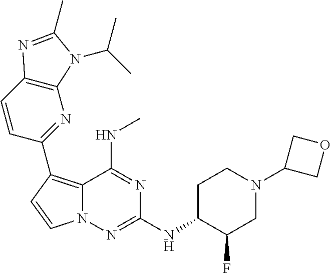 [SMILES: CNc1nc(N[C@@H]2CCN(C3COC3)C[C@H]2F)nn2ccc(-c3ccc4nc(C)n(C(C)C)c4n3)c12] |
| 154 | 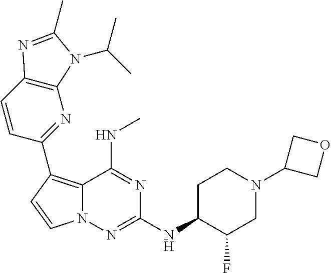 [SMILES: CNc1nc(N[C@H]2CCN(C3COC3)C[C@@H]2F)nn2ccc(-c3ccc4nc(C)n(C(C)C)c4n3)c12] |
| 155 | 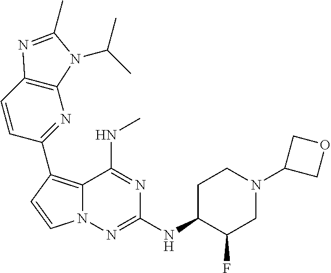 [SMILES: CNc1nc(N[C@H]2CCN(C3COC3)C[C@H]2F)nn2ccc(-c3ccc4nc(C)n(C(C)C)c4n3)c12] |
| 156 | 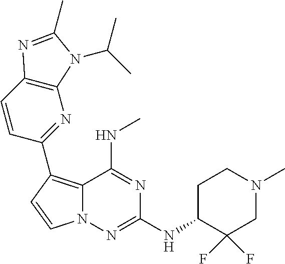 [SMILES: CNc1nc(N[C@@H]2CCN(C)CC2(F)F)nn2ccc(-c3ccc4nc(C)n(C(C)C)c4n3)c12] |
| 157 | 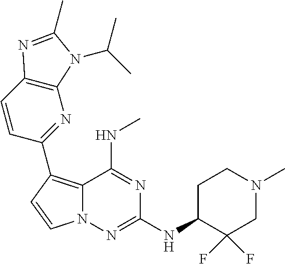 [SMILES: CNc1nc(N[C@H]2CCN(C)CC2(F)F)nn2ccc(-c3ccc4nc(C)n(C(C)C)c4n3)c12] |
| 158 | 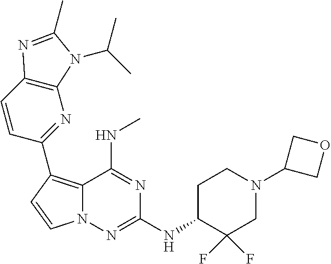 [SMILES: CNc1nc(N[C@@H]2CCN(C3COC3)CC2(F)F)nn2ccc(-c3ccc4nc(C)n(C(C)C)c4n3)c12] |
| 159 | 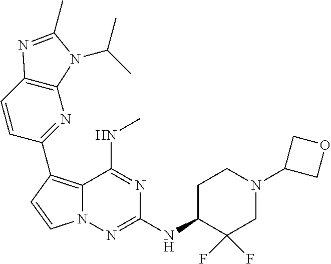 [SMILES: CNc1nc(N[C@H]2CCN(C3COC3)CC2(F)F)nn2ccc(-c3ccc4nc(C)n(C(C)C)c4n3)c12] |
| 160 | 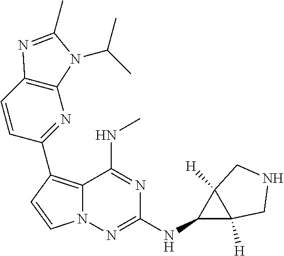 [SMILES: CNc1nc(N[C@@H]2[C@@H]3CNC[C@@H]32)nn2ccc(-c3ccc4nc(C)n(C(C)C)c4n3)c12] |
| 161 | 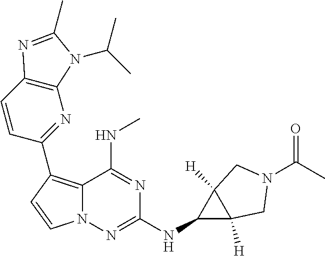 [SMILES: CNc1nc(N[C@@H]2[C@@H]3CN(C(C)=O)C[C@@H]32)nn2ccc(-c3ccc4nc(C)n(C(C)C)c4n3)c12] |
| 162 | 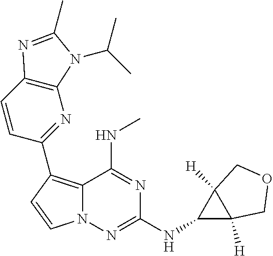 [SMILES: CNc1nc(N[C@H]2[C@@H]3COC[C@@H]32)nn2ccc(-c3ccc4nc(C)n(C(C)C)c4n3)c12] |
| 163 | 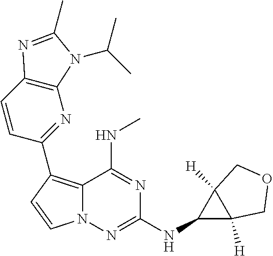 [SMILES: CNc1nc(N[C@@H]2[C@@H]3COC[C@@H]32)nn2ccc(-c3ccc4nc(C)n(C(C)C)c4n3)c12] |
| 164 | 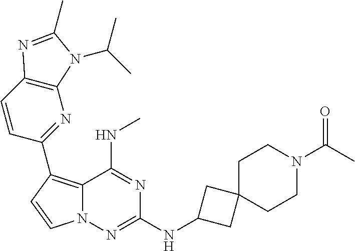 [SMILES: CNc1nc(NC2CC3(CCN(C(C)=O)CC3)C2)nn2ccc(-c3ccc4nc(C)n(C(C)C)c4n3)c12] |
| 165 | 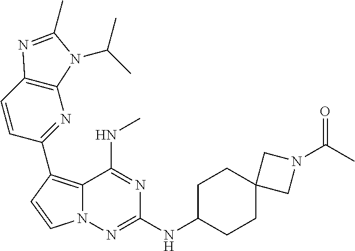 [SMILES: CNc1nc(NC2CCC3(CC2)CN(C(C)=O)C3)nn2ccc(-c3ccc4nc(C)n(C(C)C)c4n3)c12] |
| 166 | 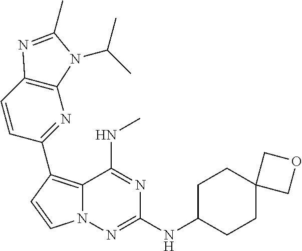 [SMILES: CNc1nc(NC2CCC3(CC2)COC3)nn2ccc(-c3ccc4nc(C)n(C(C)C)c4n3)c12] |
| 167 | 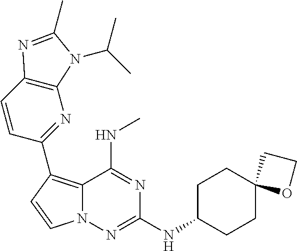 [SMILES: CNc1nc(N[C@H]2CC[C@@]3(CCO3)CC2)nn2ccc(-c3ccc4nc(C)n(C(C)C)c4n3)c12] |
| 168 | 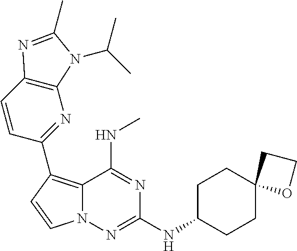 [SMILES: CNc1nc(N[C@H]2CC[C@]3(CCO3)CC2)nn2ccc(-c3ccc4nc(C)n(C(C)C)c4n3)c12] |
| 169 | 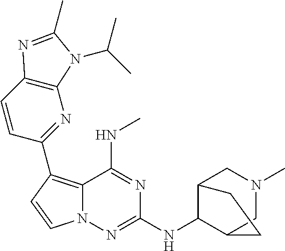 [SMILES: CNc1nc(NC2C3CCC2CN(C)C3)nn2ccc(-c3ccc4nc(C)n(C(C)C)c4n3)c12] |
| 170 | 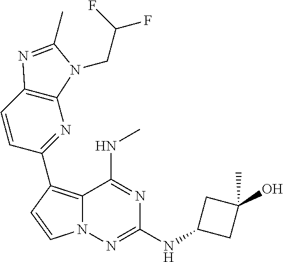 [SMILES: CNc1nc(N[C@H]2C[C@](C)(O)C2)nn2ccc(-c3ccc4nc(C)n(CC(F)F)c4n3)c12] |
| 171 | 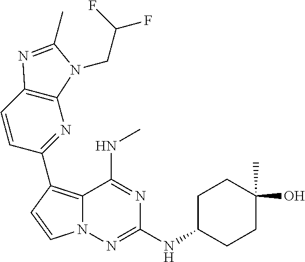 [SMILES: CNc1nc(N[C@H]2CC[C@](C)(O)CC2)nn2ccc(-c3ccc4nc(C)n(CC(F)F)c4n3)c12] |
| 172 | 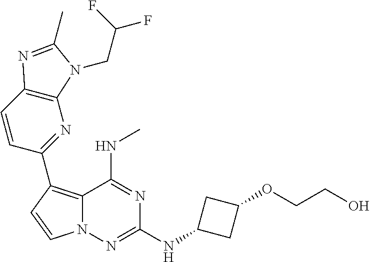 [SMILES: CNc1nc(N[C@H]2C[C@@H](OCCO)C2)nn2ccc(-c3ccc4nc(C)n(CC(F)F)c4n3)c12] |
| 173 | 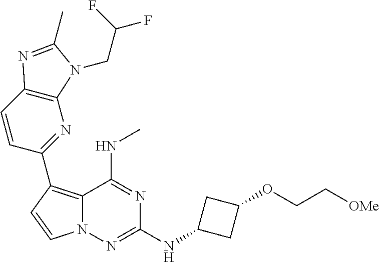 [SMILES: CNc1nc(N[C@H]2C[C@@H](OCCOC)C2)nn2ccc(-c3ccc4nc(C)n(CC(F)F)c4n3)c12] |
| 174 | 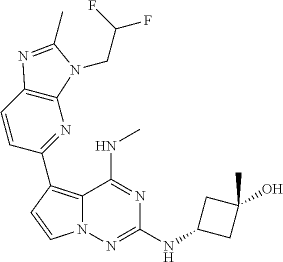 [SMILES: CNc1nc(N[C@H]2C[C@@](C)(O)C2)nn2ccc(-c3ccc4nc(C)n(CC(F)F)c4n3)c12] |
| 175 | 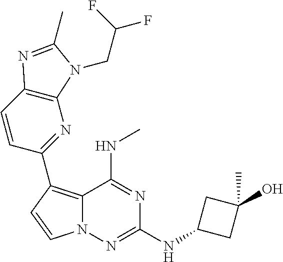 [SMILES: CNc1nc(N[C@H]2C[C@](C)(O)C2)nn2ccc(-c3ccc4nc(C)n(CC(F)F)c4n3)c12] |
| 176 | 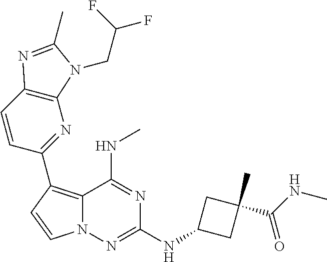 [SMILES: CNc1nc(N[C@H]2C[C@@](C)(C(=O)NC)C2)nn2ccc(-c3ccc4nc(C)n(CC(F)F)c4n3)c12] |
| 177 | 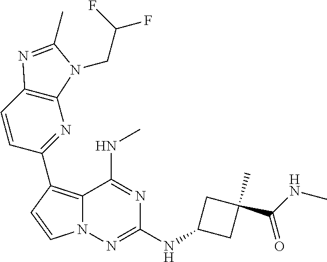 [SMILES: CNc1nc(N[C@H]2C[C@](C)(C(=O)NC)C2)nn2ccc(-c3ccc4nc(C)n(CC(F)F)c4n3)c12] |
| 178 | 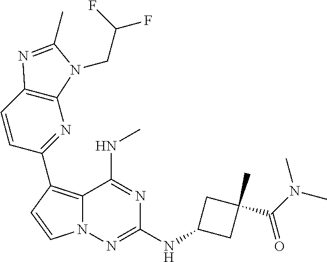 [SMILES: CNc1nc(N[C@H]2C[C@@](C)(C(=O)N(C)C)C2)nn2ccc(-c3ccc4nc(C)n(CC(F)F)c4n3)c12] |
| 179 | 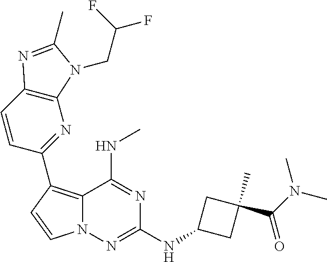 [SMILES: CNc1nc(N[C@H]2C[C@](C)(C(=O)N(C)C)C2)nn2ccc(-c3ccc4nc(C)n(CC(F)F)c4n3)c12] |
| 180 | 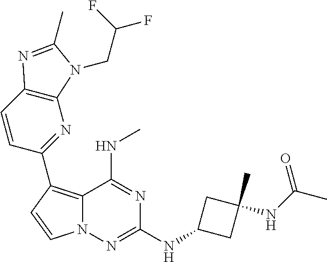 [SMILES: CNc1nc(N[C@H]2C[C@@](C)(NC(C)=O)C2)nn2ccc(-c3ccc4nc(C)n(CC(F)F)c4n3)c12] |
| 181 | 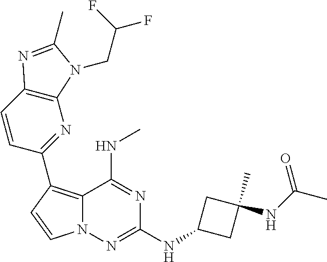 [SMILES: CNc1nc(N[C@H]2C[C@](C)(NC(C)=O)C2)nn2ccc(-c3ccc4nc(C)n(CC(F)F)c4n3)c12] |
| 182 | 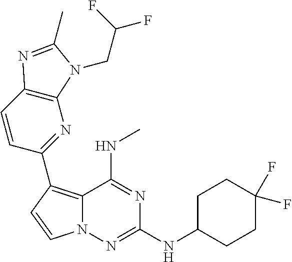 [SMILES: CNc1nc(NC2CCC(F)(F)CC2)nn2ccc(-c3ccc4nc(C)n(CC(F)F)c4n3)c12] |
| 183 | 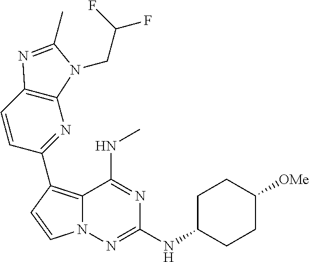 [SMILES: CNc1nc(N[C@H]2CC[C@@H](OC)CC2)nn2ccc(-c3ccc4nc(C)n(CC(F)F)c4n3)c12] |
| 184 | 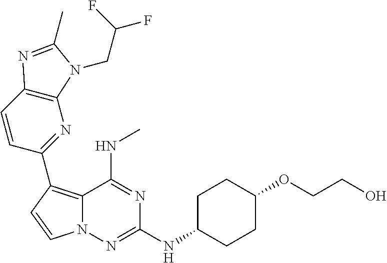 [SMILES: CNc1nc(N[C@H]2CC[C@@H](OCCO)CC2)nn2ccc(-c3ccc4nc(C)n(CC(F)F)c4n3)c12] |
| 185 | 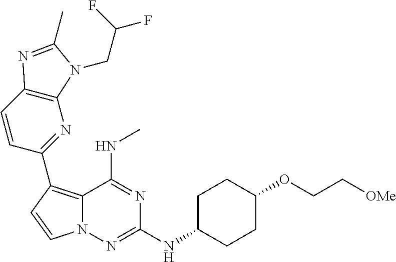 [SMILES: CNc1nc(N[C@H]2CC[C@@H](OCCOC)CC2)nn2ccc(-c3ccc4nc(C)n(CC(F)F)c4n3)c12] |
| 186 | 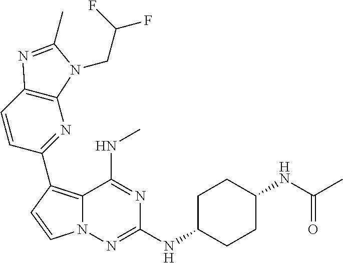 [SMILES: CNc1nc(N[C@H]2CC[C@@H](NC(C)=O)CC2)nn2ccc(-c3ccc4nc(C)n(CC(F)F)c4n3)c12] |
| 187 | 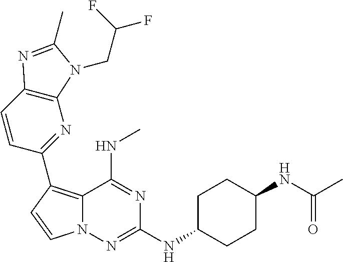 [SMILES: CNc1nc(N[C@H]2CC[C@H](NC(C)=O)CC2)nn2ccc(-c3ccc4nc(C)n(CC(F)F)c4n3)c12] |
| 188 | 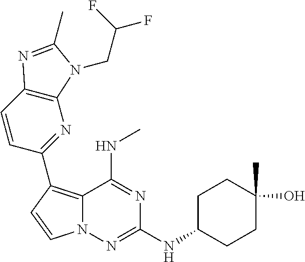 [SMILES: CNc1nc(N[C@H]2CC[C@@](C)(O)CC2)nn2ccc(-c3ccc4nc(C)n(CC(F)F)c4n3)c12] |
| 189 | 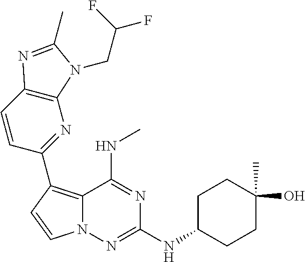 [SMILES: CNc1nc(N[C@H]2CC[C@](C)(O)CC2)nn2ccc(-c3ccc4nc(C)n(CC(F)F)c4n3)c12] |
| 190 | 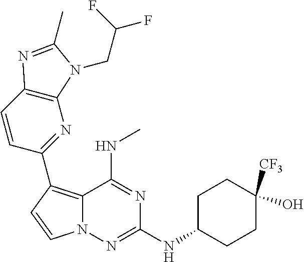 [SMILES: CNc1nc(N[C@H]2CC[C@](O)(C(F)(F)F)CC2)nn2ccc(-c3ccc4nc(C)n(CC(F)F)c4n3)c12] |
| 191 | 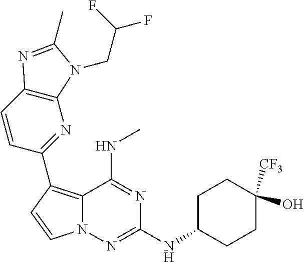 [SMILES: CNc1nc(N[C@H]2CC[C@@](O)(C(F)(F)F)CC2)nn2ccc(-c3ccc4nc(C)n(CC(F)F)c4n3)c12] |
| 192 | 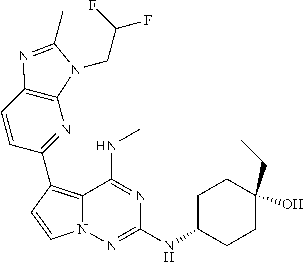 [SMILES: CC[C@]1(O)CC[C@@H](Nc2nc(NC)c3c(-c4ccc5nc(C)n(CC(F)F)c5n4)ccn3n2)CC1] |
| 193 | 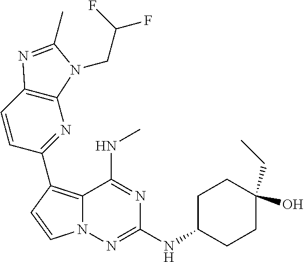 [SMILES: CC[C@]1(O)CC[C@H](Nc2nc(NC)c3c(-c4ccc5nc(C)n(CC(F)F)c5n4)ccn3n2)CC1] |
| 194 | 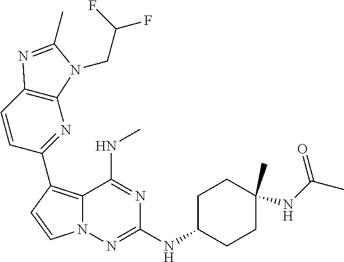 [SMILES: CNc1nc(N[C@H]2CC[C@@](C)(NC(C)=O)CC2)nn2ccc(-c3ccc4nc(C)n(CC(F)F)c4n3)c12] |
| 195 | 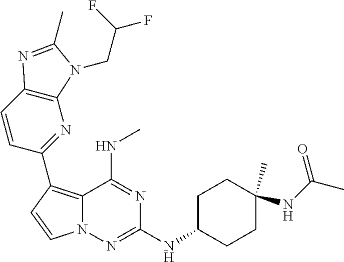 [SMILES: CNc1nc(N[C@H]2CC[C@](C)(NC(C)=O)CC2)nn2ccc(-c3ccc4nc(C)n(CC(F)F)c4n3)c12] |
| 196 | 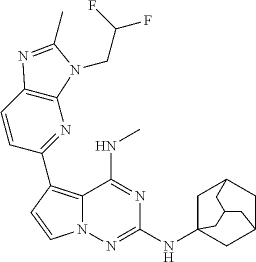 [SMILES: CNc1nc(NC23CC4CC(CC(C4)C2)C3)nn2ccc(-c3ccc4nc(C)n(CC(F)F)c4n3)c12] |
| 197 | 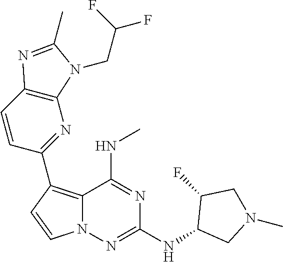 [SMILES: CNc1nc(N[C@H]2CN(C)C[C@H]2F)nn2ccc(-c3ccc4nc(C)n(CC(F)F)c4n3)c12] |
| 198 | 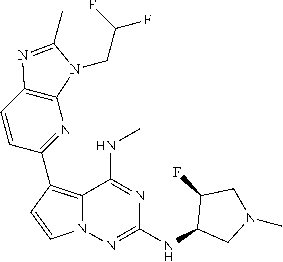 [SMILES: CNc1nc(N[C@@H]2CN(C)C[C@@H]2F)nn2ccc(-c3ccc4nc(C)n(CC(F)F)c4n3)c12] |
| 199 | 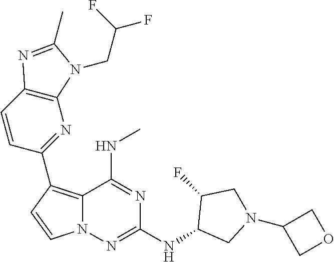 [SMILES: CNc1nc(N[C@H]2CN(C3COC3)C[C@H]2F)nn2ccc(-c3ccc4nc(C)n(CC(F)F)c4n3)c12] |
| 200 | 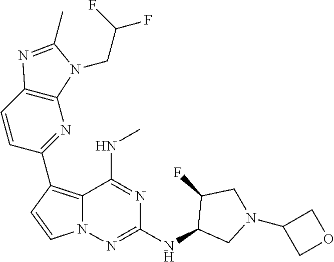 [SMILES: CNc1nc(N[C@@H]2CN(C3COC3)C[C@@H]2F)nn2ccc(-c3ccc4nc(C)n(CC(F)F)c4n3)c12] |
| 201 | 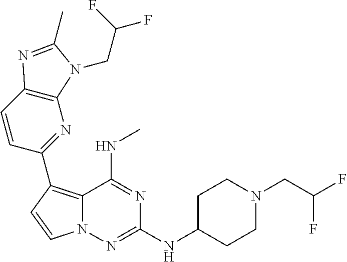 [SMILES: CNc1nc(NC2CCN(CC(F)F)CC2)nn2ccc(-c3ccc4nc(C)n(CC(F)F)c4n3)c12] |
| 202 | 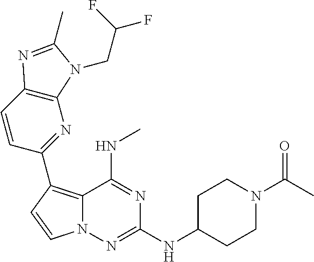 [SMILES: CNc1nc(NC2CCN(C(C)=O)CC2)nn2ccc(-c3ccc4nc(C)n(CC(F)F)c4n3)c12] |
| 203 | 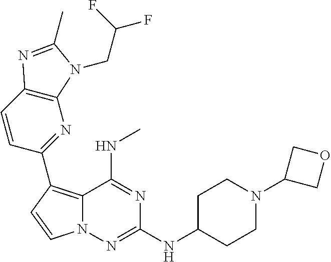 [SMILES: CNc1nc(NC2CCN(C3COC3)CC2)nn2ccc(-c3ccc4nc(C)n(CC(F)F)c4n3)c12] |
| 204 | 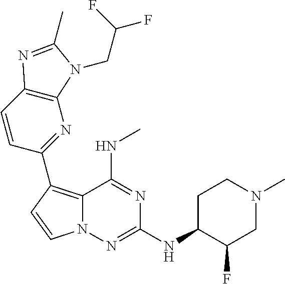 [SMILES: CNc1nc(N[C@H]2CCN(C)C[C@H]2F)nn2ccc(-c3ccc4nc(C)n(CC(F)F)c4n3)c12] |
| 205 | 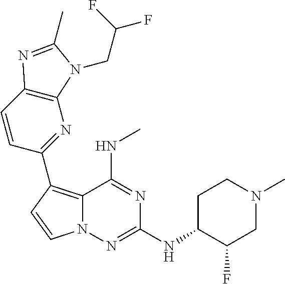 [SMILES: CNc1nc(N[C@@H]2CCN(C)C[C@@H]2F)nn2ccc(-c3ccc4nc(C)n(CC(F)F)c4n3)c12] |
| 206 | 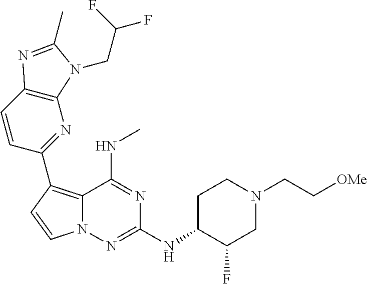 [SMILES: CNc1nc(N[C@@H]2CCN(CCOC)C[C@@H]2F)nn2ccc(-c3ccc4nc(C)n(CC(F)F)c4n3)c12] |
| 207 | 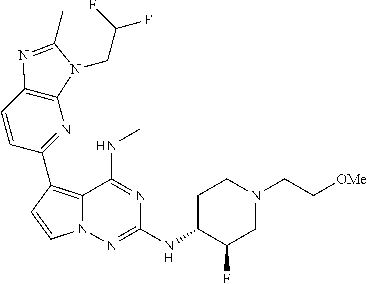 [SMILES: CNc1nc(N[C@@H]2CCN(CCOC)C[C@H]2F)nn2ccc(-c3ccc4nc(C)n(CC(F)F)c4n3)c12] |
| 208 | 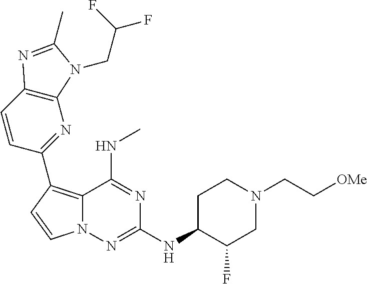 [SMILES: CNc1nc(N[C@H]2CCN(CCOC)C[C@@H]2F)nn2ccc(-c3ccc4nc(C)n(CC(F)F)c4n3)c12] |
| 209 | 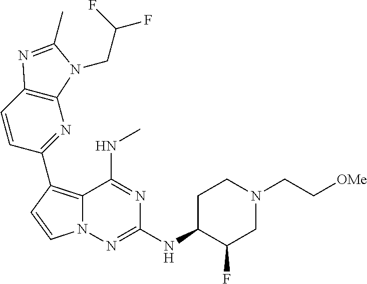 [SMILES: CNc1nc(N[C@H]2CCN(CCOC)C[C@H]2F)nn2ccc(-c3ccc4nc(C)n(CC(F)F)c4n3)c12] |
| 210 | 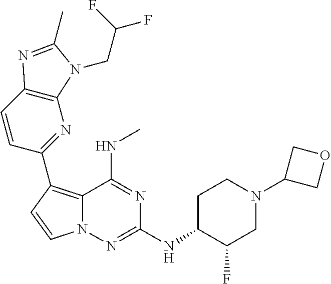 [SMILES: CNc1nc(N[C@@H]2CCN(C3COC3)C[C@@H]2F)nn2ccc(-c3ccc4nc(C)n(CC(F)F)c4n3)c12] |
| 211 | 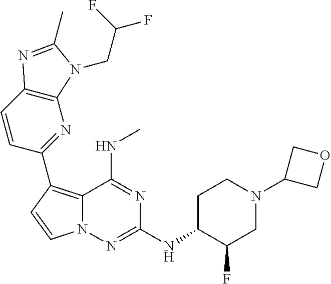 [SMILES: CNc1nc(N[C@@H]2CCN(C3COC3)C[C@H]2F)nn2ccc(-c3ccc4nc(C)n(CC(F)F)c4n3)c12] |
| 212 | 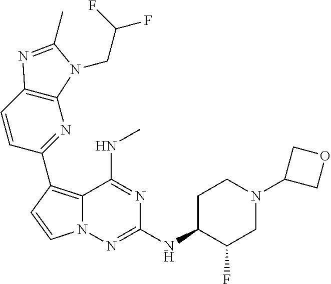 [SMILES: CNc1nc(N[C@H]2CCN(C3COC3)C[C@@H]2F)nn2ccc(-c3ccc4nc(C)n(CC(F)F)c4n3)c12] |
| 213 | 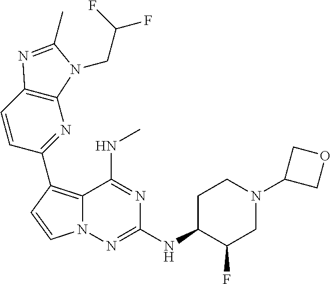 [SMILES: CNc1nc(N[C@H]2CCN(C3COC3)C[C@H]2F)nn2ccc(-c3ccc4nc(C)n(CC(F)F)c4n3)c12] |
| 214 | 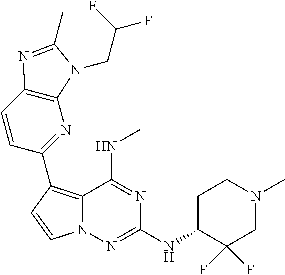 [SMILES: CNc1nc(N[C@@H]2CCN(C)CC2(F)F)nn2ccc(-c3ccc4nc(C)n(CC(F)F)c4n3)c12] |
| 215 | 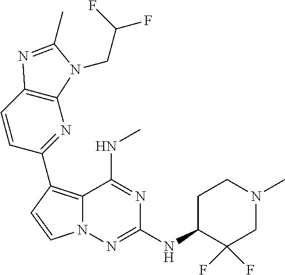 [SMILES: CNc1nc(N[C@H]2CCN(C)CC2(F)F)nn2ccc(-c3ccc4nc(C)n(CC(F)F)c4n3)c12] |
| 216 | 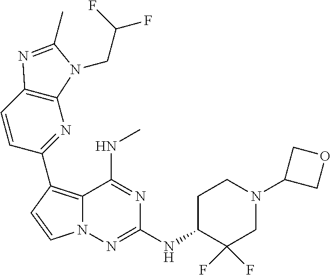 [SMILES: CNc1nc(N[C@@H]2CCN(C3COC3)CC2(F)F)nn2ccc(-c3ccc4nc(C)n(CC(F)F)c4n3)c12] |
| 217 | 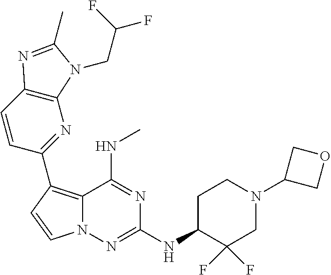 [SMILES: CNc1nc(N[C@H]2CCN(C3COC3)CC2(F)F)nn2ccc(-c3ccc4nc(C)n(CC(F)F)c4n3)c12] |
| 218 | 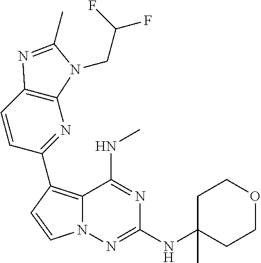 [SMILES: CNc1nc(NC2(C)CCOCC2)nn2ccc(-c3ccc4nc(C)n(CC(F)F)c4n3)c12] |
| 219 | 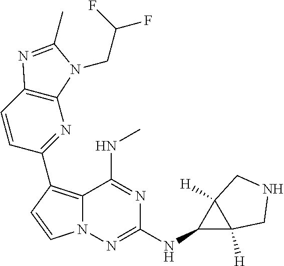 [SMILES: CNc1nc(N[C@@H]2[C@@H]3CNC[C@@H]32)nn2ccc(-c3ccc4nc(C)n(CC(F)F)c4n3)c12] |
| 220 | 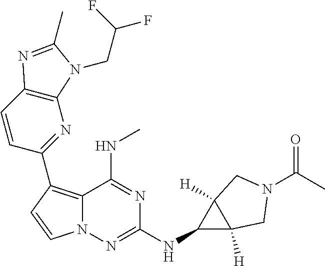 [SMILES: CNc1nc(N[C@@H]2[C@@H]3CN(C(C)=O)C[C@@H]32)nn2ccc(-c3ccc4nc(C)n(CC(F)F)c4n3)c12] |
| 221 | 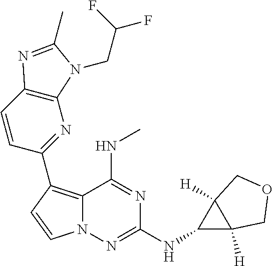 [SMILES: CNc1nc(N[C@H]2[C@@H]3COC[C@@H]32)nn2ccc(-c3ccc4nc(C)n(CC(F)F)c4n3)c12] |
| 222 | 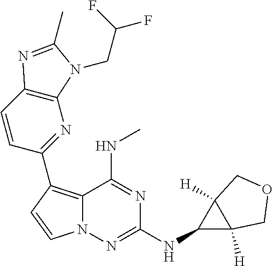 [SMILES: CNc1nc(N[C@@H]2[C@@H]3COC[C@@H]32)nn2ccc(-c3ccc4nc(C)n(CC(F)F)c4n3)c12] |
| 223 | 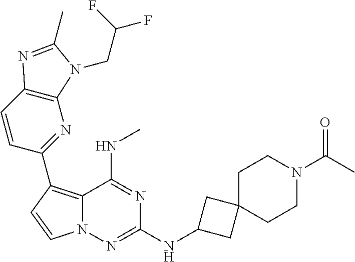 [SMILES: CNc1nc(NC2CC3(CCN(C(C)=O)CC3)C2)nn2ccc(-c3ccc4nc(C)n(CC(F)F)c4n3)c12] |
| 224 | 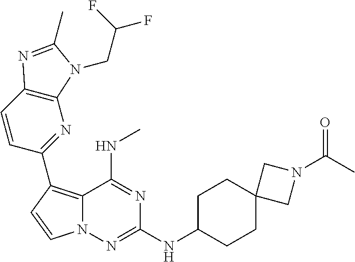 [SMILES: CNc1nc(NC2CCC3(CC2)CN(C(C)=O)C3)nn2ccc(-c3ccc4nc(C)n(CC(F)F)c4n3)c12] |
| 225 | 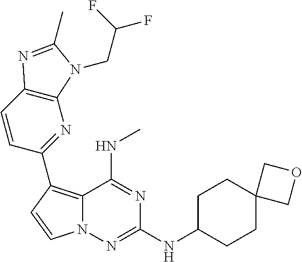 [SMILES: CNc1nc(NC2CCC3(CC2)COC3)nn2ccc(-c3ccc4nc(C)n(CC(F)F)c4n3)c12] |
| 226 | 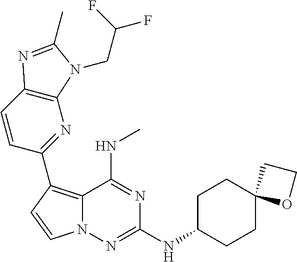 [SMILES: CNc1nc(N[C@H]2CC[C@@]3(CCO3)CC2)nn2ccc(-c3ccc4nc(C)n(CC(F)F)c4n3)c12] |
| 227 | 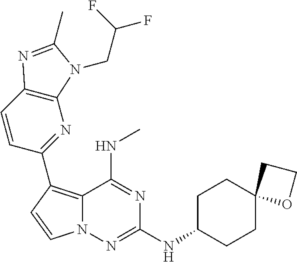 [SMILES: CNc1nc(N[C@H]2CC[C@]3(CCO3)CC2)nn2ccc(-c3ccc4nc(C)n(CC(F)F)c4n3)c12] |
| 228 | 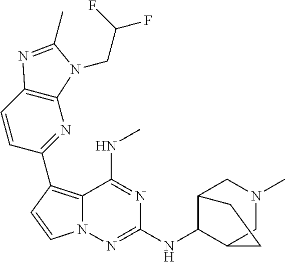 [SMILES: CNc1nc(NC2C3CCC2CN(C)C3)nn2ccc(-c3ccc4nc(C)n(CC(F)F)c4n3)c12] |
| 229 | 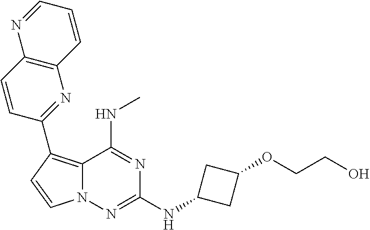 [SMILES: CNc1nc(N[C@H]2C[C@@H](OCCO)C2)nn2ccc(-c3ccc4ncccc4n3)c12] |
| 230 | 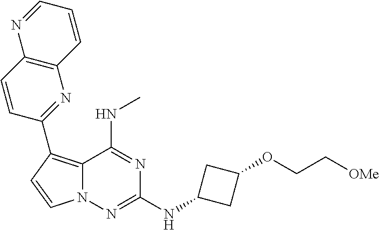 [SMILES: CNc1nc(N[C@H]2C[C@@H](OCCOC)C2)nn2ccc(-c3ccc4ncccc4n3)c12] |
| 231 | 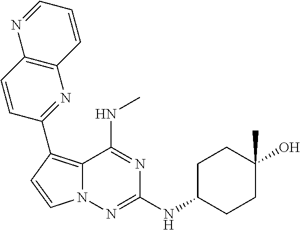 [SMILES: CNc1nc(N[C@H]2CC[C@@](C)(O)CC2)nn2ccc(-c3ccc4ncccc4n3)c12] |
| 232 | 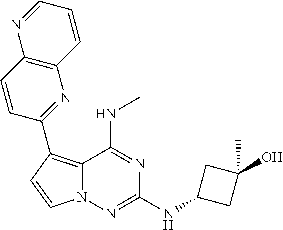 [SMILES: CNc1nc(N[C@H]2C[C@](C)(O)C2)nn2ccc(-c3ccc4ncccc4n3)c12] |
| 233 | 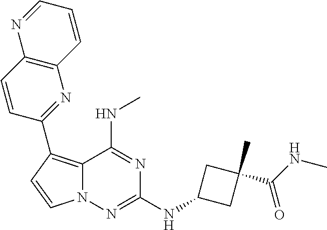 [SMILES: CNc1nc(N[C@H]2C[C@@](C)(C(=O)NC)C2)nn2ccc(-c3ccc4ncccc4n3)c12] |
| 234 | 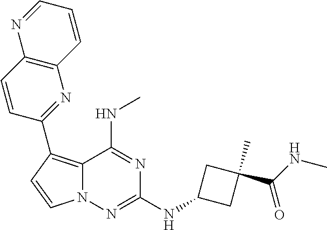 [SMILES: CNc1nc(N[C@H]2C[C@](C)(C(=O)NC)C2)nn2ccc(-c3ccc4ncccc4n3)c12] |
| 235 | 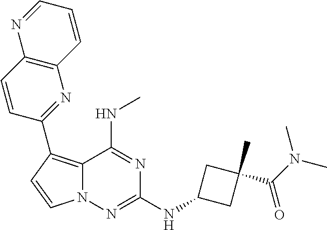 [SMILES: CNc1nc(N[C@H]2C[C@@](C)(C(=O)N(C)C)C2)nn2ccc(-c3ccc4ncccc4n3)c12] |
| 236 | 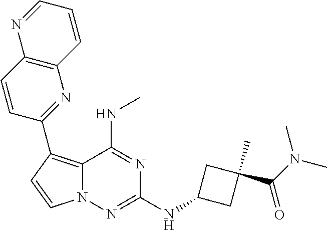 [SMILES: CNc1nc(N[C@H]2C[C@](C)(C(=O)N(C)C)C2)nn2ccc(-c3ccc4ncccc4n3)c12] |
| 237 | 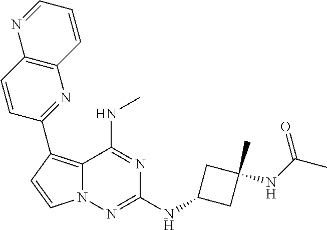 [SMILES: CNc1nc(N[C@H]2C[C@@](C)(NC(C)=O)C2)nn2ccc(-c3ccc4ncccc4n3)c12] |
| 238 | 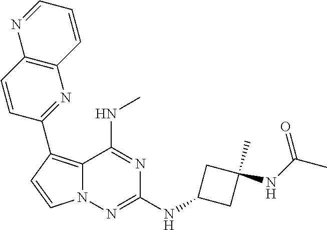 [SMILES: CNc1nc(N[C@H]2C[C@](C)(NC(C)=O)C2)nn2ccc(-c3ccc4ncccc4n3)c12] |
| 239 | 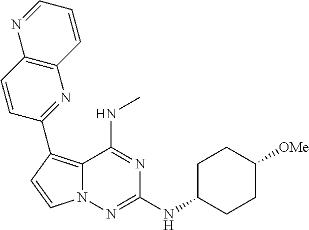 [SMILES: CNc1nc(N[C@H]2CC[C@@H](OC)CC2)nn2ccc(-c3ccc4ncccc4n3)c12] |
| 240 | 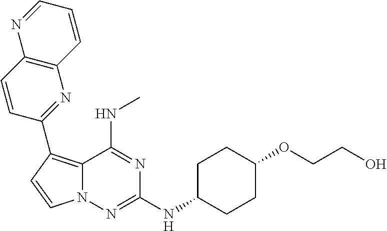 [SMILES: CNc1nc(N[C@H]2CC[C@@H](OCCO)CC2)nn2ccc(-c3ccc4ncccc4n3)c12] |
| 241 | 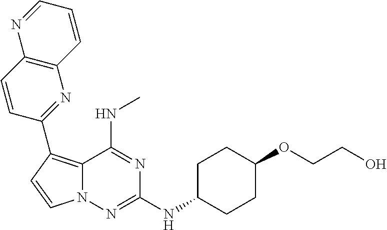 [SMILES: CNc1nc(N[C@H]2CC[C@H](OCCO)CC2)nn2ccc(-c3ccc4ncccc4n3)c12] |
| 242 | 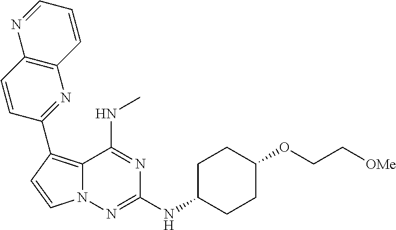 [SMILES: CNc1nc(N[C@H]2CC[C@@H](OCCOC)CC2)nn2ccc(-c3ccc4ncccc4n3)c12] |
| 243 | 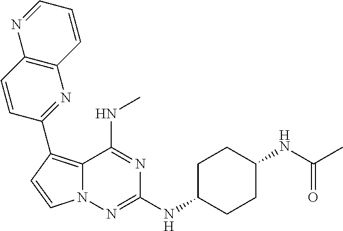 [SMILES: CNc1nc(N[C@H]2CC[C@@H](NC(C)=O)CC2)nn2ccc(-c3ccc4ncccc4n3)c12] |
| 244 | 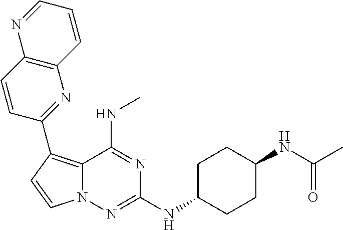 [SMILES: CNc1nc(N[C@H]2CC[C@H](NC(C)=O)CC2)nn2ccc(-c3ccc4ncccc4n3)c12] |
| 245 | 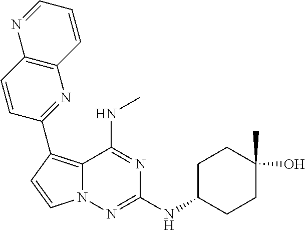 [SMILES: CNc1nc(N[C@H]2CC[C@@](C)(O)CC2)nn2ccc(-c3ccc4ncccc4n3)c12] |
| 246 | 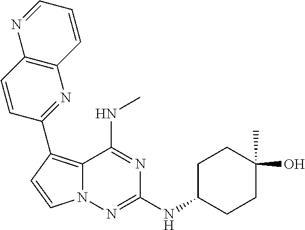 [SMILES: CNc1nc(N[C@H]2CC[C@](C)(O)CC2)nn2ccc(-c3ccc4ncccc4n3)c12] |
| 247 | 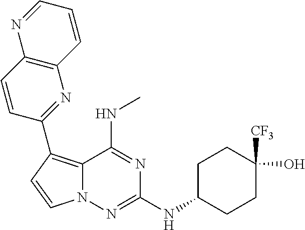 [SMILES: CNc1nc(N[C@H]2CC[C@](O)(C(F)(F)F)CC2)nn2ccc(-c3ccc4ncccc4n3)c12] |
| 248 | 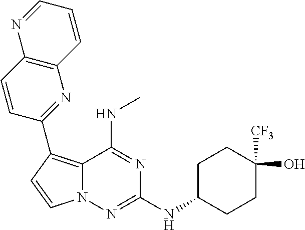 [SMILES: CNc1nc(N[C@H]2CC[C@@](O)(C(F)(F)F)CC2)nn2ccc(-c3ccc4ncccc4n3)c12] |
| 249 | 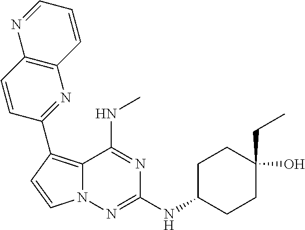 [SMILES: CC[C@]1(O)CC[C@@H](Nc2nc(NC)c3c(-c4ccc5ncccc5n4)ccn3n2)CC1] |
| 250 | 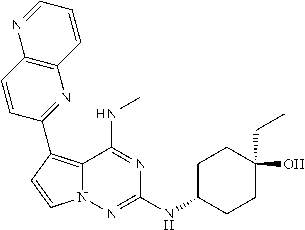 [SMILES: CC[C@]1(O)CC[C@H](Nc2nc(NC)c3c(-c4ccc5ncccc5n4)ccn3n2)CC1] |
| 251 | 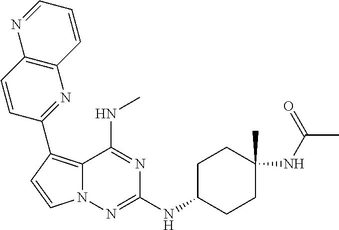 [SMILES: CNc1nc(N[C@H]2CC[C@@](C)(NC(C)=O)CC2)nn2ccc(-c3ccc4ncccc4n3)c12] |
| 252 | 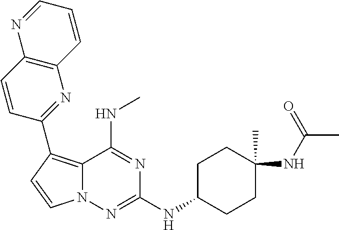 [SMILES: CNc1nc(N[C@H]2CC[C@](C)(NC(C)=O)CC2)nn2ccc(-c3ccc4ncccc4n3)c12] |
| 253 | 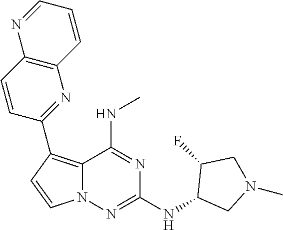 [SMILES: CNc1nc(N[C@H]2CN(C)C[C@H]2F)nn2ccc(-c3ccc4ncccc4n3)c12] |
| 254 | 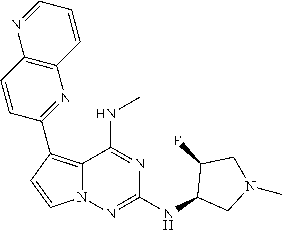 [SMILES: CNc1nc(N[C@@H]2CN(C)C[C@@H]2F)nn2ccc(-c3ccc4ncccc4n3)c12] |
| 255 | 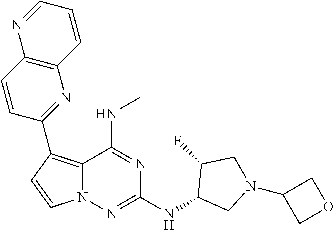 [SMILES: CNc1nc(N[C@H]2CN(C3COC3)C[C@H]2F)nn2ccc(-c3ccc4ncccc4n3)c12] |
| 256 | 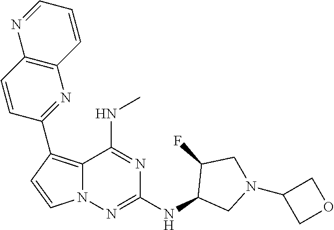 [SMILES: CNc1nc(N[C@@H]2CN(C3COC3)C[C@@H]2F)nn2ccc(-c3ccc4ncccc4n3)c12] |
| 257 | 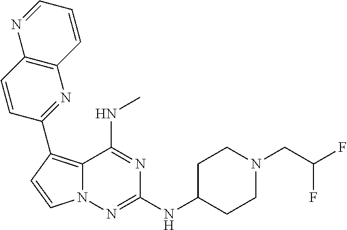 [SMILES: CNc1nc(NC2CCN(CC(F)F)CC2)nn2ccc(-c3ccc4ncccc4n3)c12] |
| 258 | 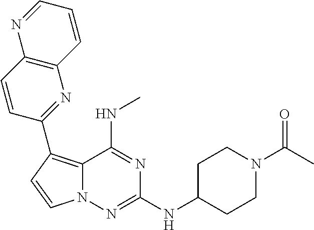 [SMILES: CNc1nc(NC2CCN(C(C)=O)CC2)nn2ccc(-c3ccc4ncccc4n3)c12] |
| 259 | 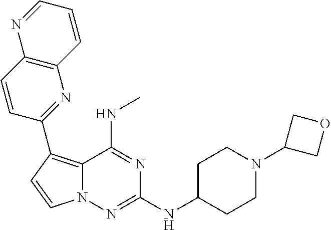 [SMILES: CNc1nc(NC2CCN(C3COC3)CC2)nn2ccc(-c3ccc4ncccc4n3)c12] |
| 260 | 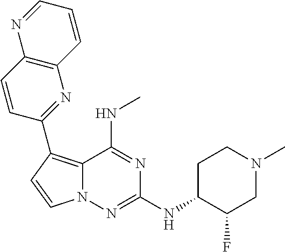 [SMILES: CNc1nc(N[C@@H]2CCN(C)C[C@@H]2F)nn2ccc(-c3ccc4ncccc4n3)c12] |
| 261 | 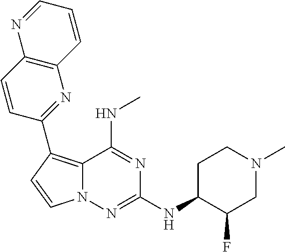 [SMILES: CNc1nc(N[C@H]2CCN(C)C[C@H]2F)nn2ccc(-c3ccc4ncccc4n3)c12] |
| 262 | 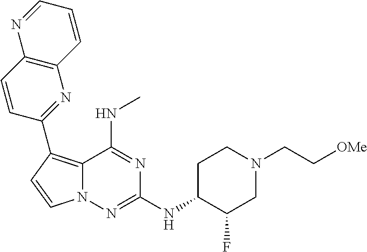 [SMILES: CNc1nc(N[C@@H]2CCN(CCOC)C[C@@H]2F)nn2ccc(-c3ccc4ncccc4n3)c12] |
| 263 | 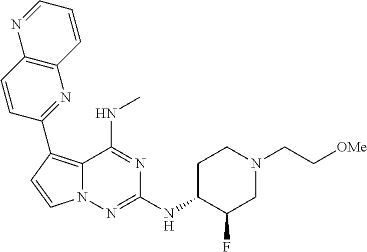 [SMILES: CNc1nc(N[C@@H]2CCN(CCOC)C[C@H]2F)nn2ccc(-c3ccc4ncccc4n3)c12] |
| 264 | 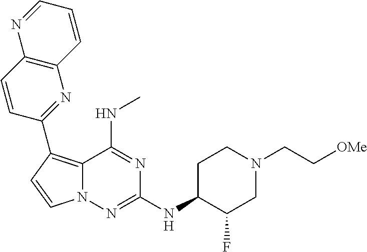 [SMILES: CNc1nc(N[C@H]2CCN(CCOC)C[C@@H]2F)nn2ccc(-c3ccc4ncccc4n3)c12] |
| 265 | 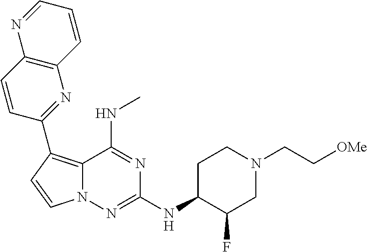 [SMILES: CNc1nc(N[C@H]2CCN(CCOC)C[C@H]2F)nn2ccc(-c3ccc4ncccc4n3)c12] |
| 266 | 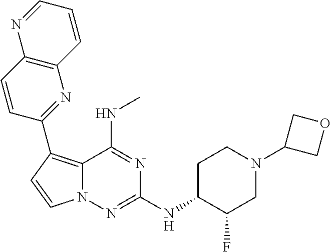 [SMILES: CNc1nc(N[C@@H]2CCN(C3COC3)C[C@@H]2F)nn2ccc(-c3ccc4ncccc4n3)c12] |
| 267 | 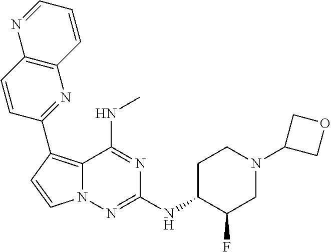 [SMILES: CNc1nc(N[C@@H]2CCN(C3COC3)C[C@H]2F)nn2ccc(-c3ccc4ncccc4n3)c12] |
| 268 | 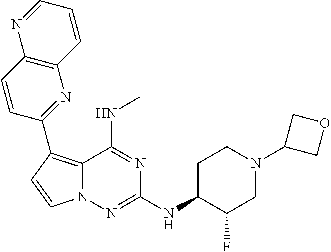 [SMILES: CNc1nc(N[C@H]2CCN(C3COC3)C[C@@H]2F)nn2ccc(-c3ccc4ncccc4n3)c12] |
| 269 | 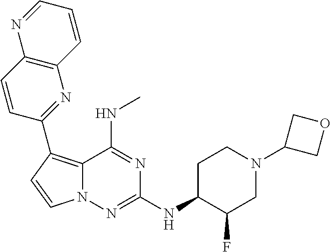 [SMILES: CNc1nc(N[C@H]2CCN(C3COC3)C[C@H]2F)nn2ccc(-c3ccc4ncccc4n3)c12] |
| 270 | 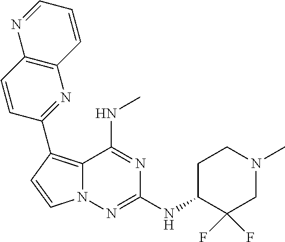 [SMILES: CNc1nc(N[C@@H]2CCN(C)CC2(F)F)nn2ccc(-c3ccc4ncccc4n3)c12] |
| 271 | 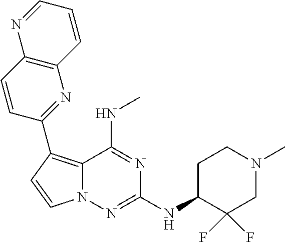 [SMILES: CNc1nc(N[C@H]2CCN(C)CC2(F)F)nn2ccc(-c3ccc4ncccc4n3)c12] |
| 272 | 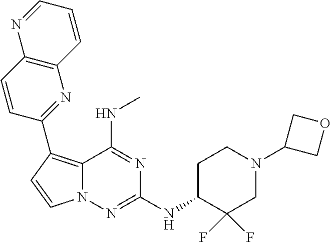 [SMILES: CNc1nc(N[C@@H]2CCN(C3COC3)CC2(F)F)nn2ccc(-c3ccc4ncccc4n3)c12] |
| 273 | 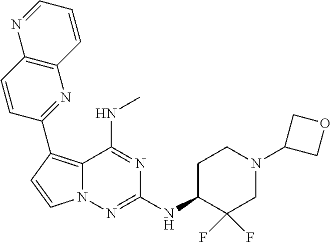 [SMILES: CNc1nc(N[C@H]2CCN(C3COC3)CC2(F)F)nn2ccc(-c3ccc4ncccc4n3)c12] |
| 274 | 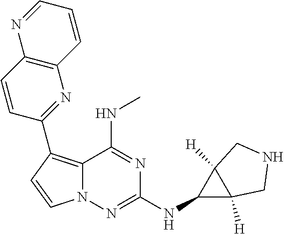 [SMILES: CNc1nc(N[C@@H]2[C@@H]3CNC[C@@H]32)nn2ccc(-c3ccc4ncccc4n3)c12] |
| 275 | 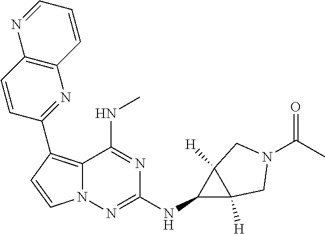 [SMILES: CNc1nc(N[C@@H]2[C@@H]3CN(C(C)=O)C[C@@H]32)nn2ccc(-c3ccc4ncccc4n3)c12] |
| 276 | 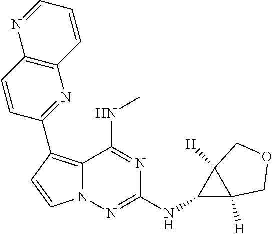 [SMILES: CNc1nc(N[C@H]2[C@@H]3COC[C@@H]32)nn2ccc(-c3ccc4ncccc4n3)c12] |
| 277 | 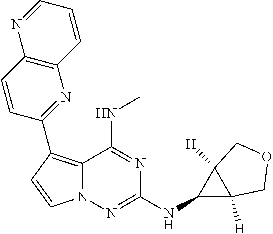 [SMILES: CNc1nc(N[C@@H]2[C@@H]3COC[C@@H]32)nn2ccc(-c3ccc4ncccc4n3)c12] |
| 278 | 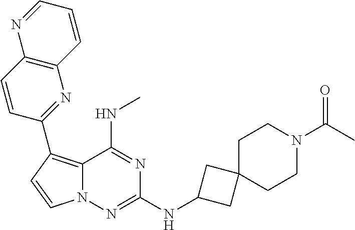 [SMILES: CNc1nc(NC2CC3(CCN(C(C)=O)CC3)C2)nn2ccc(-c3ccc4ncccc4n3)c12] |
| 279 | 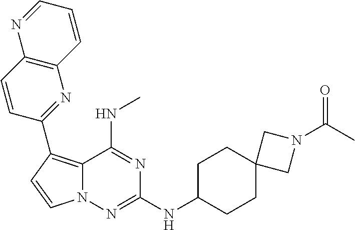 [SMILES: CNc1nc(NC2CCC3(CC2)CN(C(C)=O)C3)nn2ccc(-c3ccc4ncccc4n3)c12] |
| 280 | 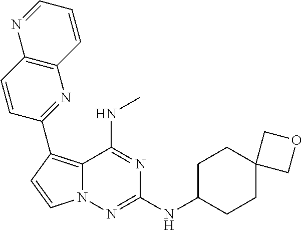 [SMILES: CNc1nc(NC2CCC3(CC2)COC3)nn2ccc(-c3ccc4ncccc4n3)c12] |
| 281 | 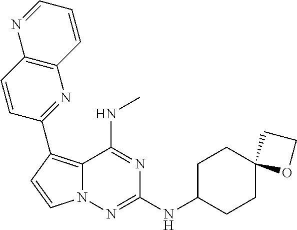 |
| 282 | 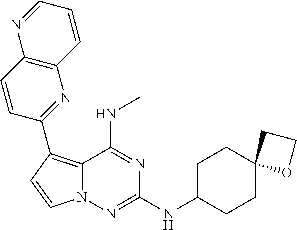 |
| 283 | 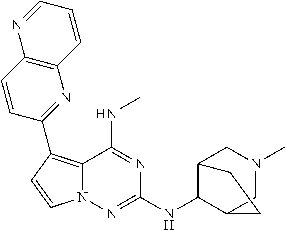 [SMILES: CNc1nc(NC2C3CCC2CN(C)C3)nn2ccc(-c3ccc4ncccc4n3)c12] |
| 284 | 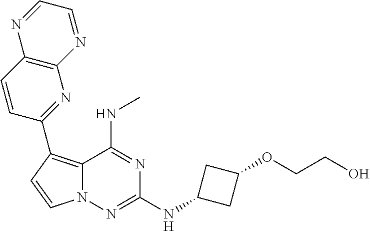 [SMILES: CNc1nc(N[C@H]2C[C@@H](OCCO)C2)nn2ccc(-c3ccc4nccnc4n3)c12] |
| 285 | 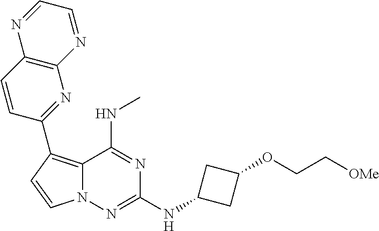 [SMILES: CNc1nc(N[C@H]2C[C@@H](OCCOC)C2)nn2ccc(-c3ccc4nccnc4n3)c12] |
| 286 | 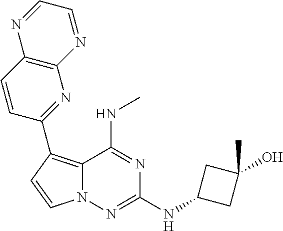 [SMILES: CNc1nc(N[C@H]2C[C@@](C)(O)C2)nn2ccc(-c3ccc4nccnc4n3)c12] |
| 287 | 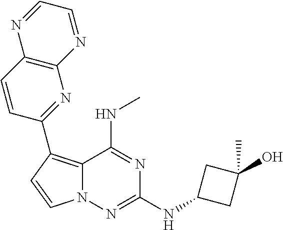 [SMILES: CNc1nc(N[C@H]2C[C@](C)(O)C2)nn2ccc(-c3ccc4nccnc4n3)c12] |
| 288 | 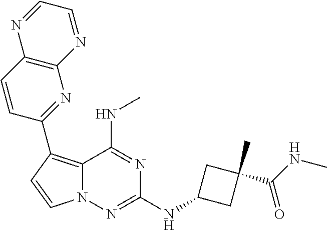 [SMILES: CNc1nc(N[C@H]2C[C@@](C)(C(=O)NC)C2)nn2ccc(-c3ccc4nccnc4n3)c12] |
| 289 | 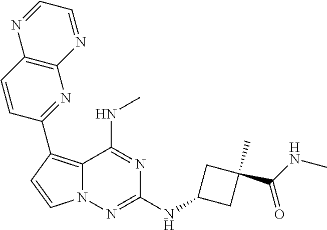 [SMILES: CNc1nc(N[C@H]2C[C@](C)(C(=O)NC)C2)nn2ccc(-c3ccc4nccnc4n3)c12] |
| 290 | 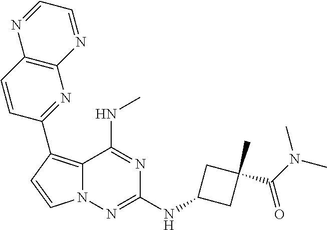 [SMILES: CNc1nc(N[C@H]2C[C@@](C)(C(=O)N(C)C)C2)nn2ccc(-c3ccc4nccnc4n3)c12] |
| 291 | 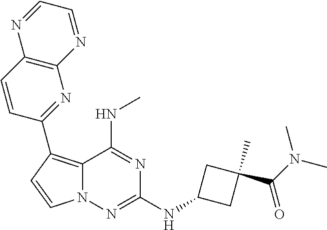 [SMILES: CNc1nc(N[C@H]2C[C@](C)(C(=O)N(C)C)C2)nn2ccc(-c3ccc4nccnc4n3)c12] |
| 292 | 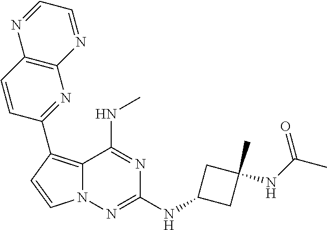 [SMILES: CNc1nc(N[C@H]2C[C@@](C)(NC(C)=O)C2)nn2ccc(-c3ccc4nccnc4n3)c12] |
| 293 | 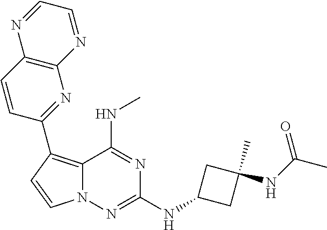 [SMILES: CNc1nc(N[C@H]2C[C@](C)(NC(C)=O)C2)nn2ccc(-c3ccc4nccnc4n3)c12] |
| 294 | 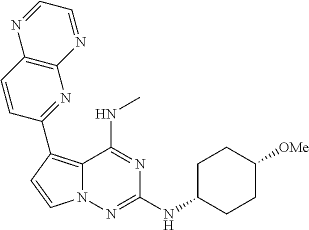 [SMILES: CNc1nc(N[C@H]2CC[C@@H](OC)CC2)nn2ccc(-c3ccc4nccnc4n3)c12] |
| 295 | 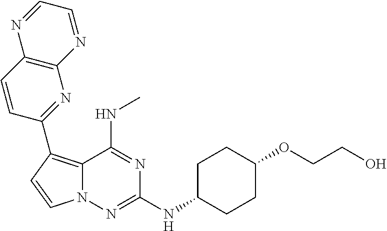 [SMILES: CNc1nc(N[C@H]2CC[C@@H](OCCO)CC2)nn2ccc(-c3ccc4nccnc4n3)c12] |
| 296 | 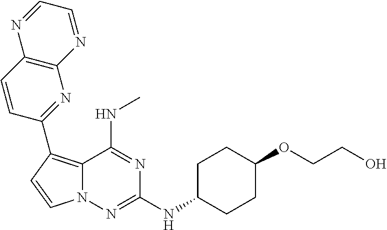 [SMILES: CNc1nc(N[C@H]2CC[C@H](OCCO)CC2)nn2ccc(-c3ccc4nccnc4n3)c12] |
| 297 | 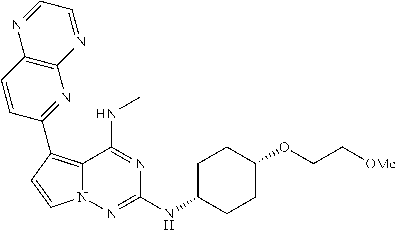 [SMILES: CNc1nc(N[C@H]2CC[C@@H](OCCOC)CC2)nn2ccc(-c3ccc4nccnc4n3)c12] |
| 298 | 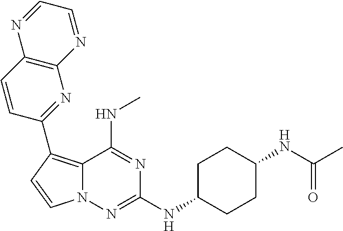 [SMILES: CNc1nc(N[C@H]2CC[C@@H](NC(C)=O)CC2)nn2ccc(-c3ccc4nccnc4n3)c12] |
| 299 | 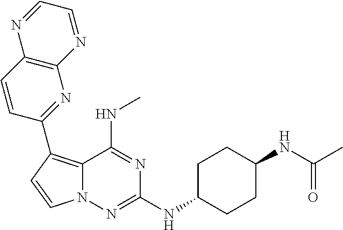 [SMILES: CNc1nc(N[C@H]2CC[C@H](NC(C)=O)CC2)nn2ccc(-c3ccc4nccnc4n3)c12] |
| 300 | 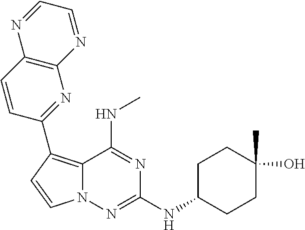 [SMILES: CNc1nc(N[C@H]2CC[C@@](C)(O)CC2)nn2ccc(-c3ccc4nccnc4n3)c12] |
| 301 | 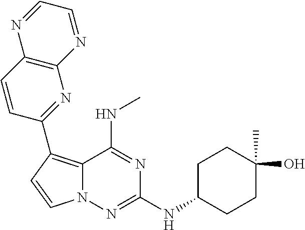 [SMILES: CNc1nc(N[C@H]2CC[C@](C)(O)CC2)nn2ccc(-c3ccc4nccnc4n3)c12] |
| 302 | 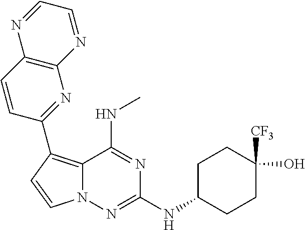 [SMILES: CNc1nc(N[C@H]2CC[C@](O)(C(F)(F)F)CC2)nn2ccc(-c3ccc4nccnc4n3)c12] |
| 303 | 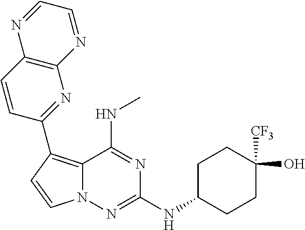 [SMILES: CNc1nc(N[C@H]2CC[C@@](O)(C(F)(F)F)CC2)nn2ccc(-c3ccc4nccnc4n3)c12] |
| 304 | 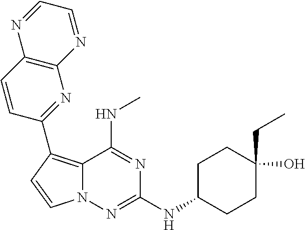 [SMILES: CC[C@]1(O)CC[C@@H](Nc2nc(NC)c3c(-c4ccc5nccnc5n4)ccn3n2)CC1] |
| 305 | 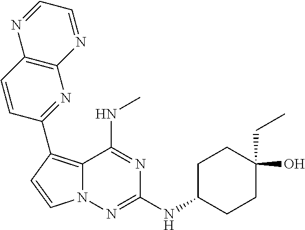 [SMILES: CC[C@]1(O)CC[C@H](Nc2nc(NC)c3c(-c4ccc5nccnc5n4)ccn3n2)CC1] |
| 306 | 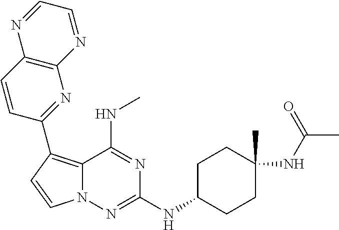 [SMILES: CNc1nc(N[C@H]2CC[C@@](C)(NC(C)=O)CC2)nn2ccc(-c3ccc4nccnc4n3)c12] |
| 307 | 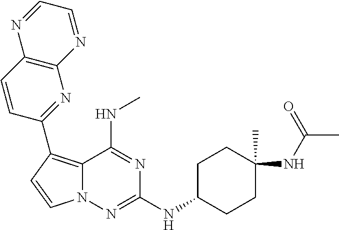 [SMILES: CNc1nc(N[C@H]2CC[C@](C)(NC(C)=O)CC2)nn2ccc(-c3ccc4nccnc4n3)c12] |
| 308 | 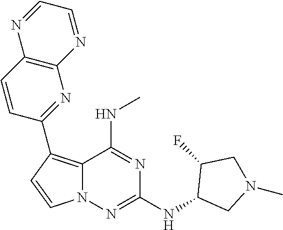 [SMILES: CNc1nc(N[C@H]2CN(C)C[C@H]2F)nn2ccc(-c3ccc4nccnc4n3)c12] |
| 309 | 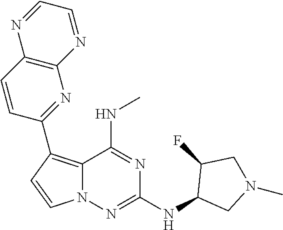 [SMILES: CNc1nc(N[C@@H]2CN(C)C[C@@H]2F)nn2ccc(-c3ccc4nccnc4n3)c12] |
| 310 | 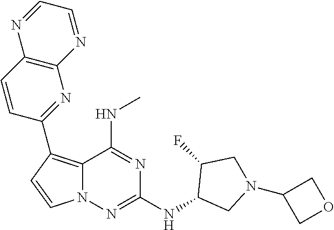 [SMILES: CNc1nc(N[C@H]2CN(C3COC3)C[C@H]2F)nn2ccc(-c3ccc4nccnc4n3)c12] |
| 311 | 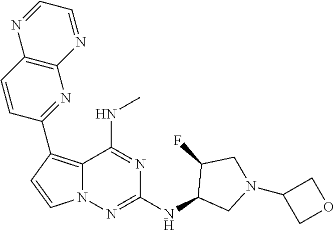 [SMILES: CNc1nc(N[C@@H]2CN(C3COC3)C[C@@H]2F)nn2ccc(-c3ccc4nccnc4n3)c12] |
| 312 | 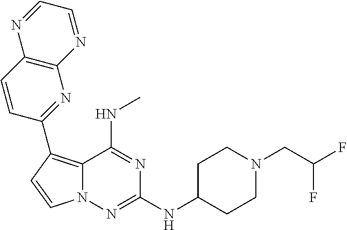 [SMILES: CNc1nc(NC2CCN(CC(F)F)CC2)nn2ccc(-c3ccc4nccnc4n3)c12] |
| 313 | 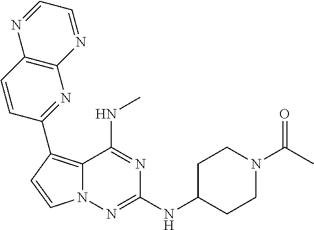 [SMILES: CNc1nc(NC2CCN(C(C)=O)CC2)nn2ccc(-c3ccc4nccnc4n3)c12] |
| 314 | 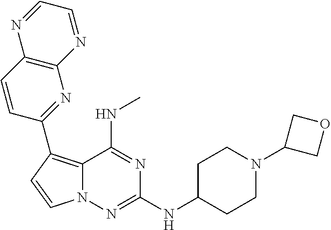 [SMILES: CNc1nc(NC2CCN(C3COC3)CC2)nn2ccc(-c3ccc4nccnc4n3)c12] |
| 315 | 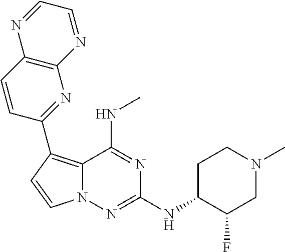 [SMILES: CNc1nc(N[C@@H]2CCN(C)C[C@@H]2F)nn2ccc(-c3ccc4nccnc4n3)c12] |
| 316 | 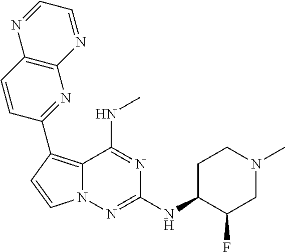 [SMILES: CNc1nc(N[C@H]2CCN(C)C[C@H]2F)nn2ccc(-c3ccc4nccnc4n3)c12] |
| 317 | 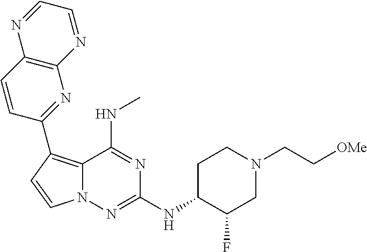 [SMILES: CNc1nc(N[C@@H]2CCN(CCOC)C[C@@H]2F)nn2ccc(-c3ccc4nccnc4n3)c12] |
| 318 | 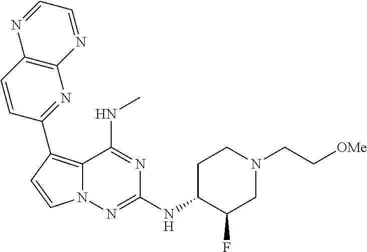 [SMILES: CNc1nc(N[C@@H]2CCN(CCOC)C[C@H]2F)nn2ccc(-c3ccc4nccnc4n3)c12] |
| 319 | 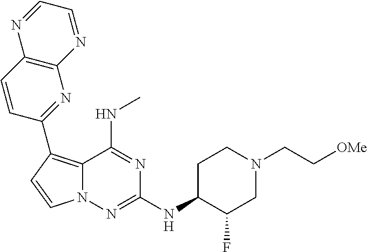 [SMILES: CNc1nc(N[C@H]2CCN(CCOC)C[C@@H]2F)nn2ccc(-c3ccc4nccnc4n3)c12] |
| 320 | 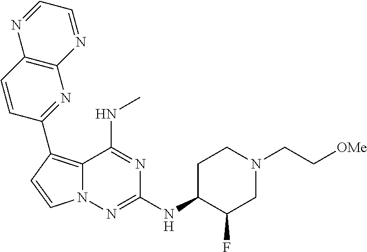 [SMILES: CNc1nc(N[C@H]2CCN(CCOC)C[C@H]2F)nn2ccc(-c3ccc4nccnc4n3)c12] |
| 321 | 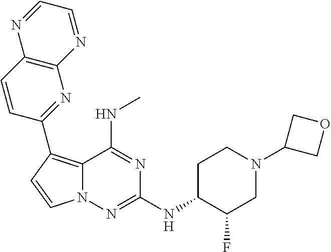 [SMILES: CNc1nc(N[C@@H]2CCN(C3COC3)C[C@@H]2F)nn2ccc(-c3ccc4nccnc4n3)c12] |
| 322 | 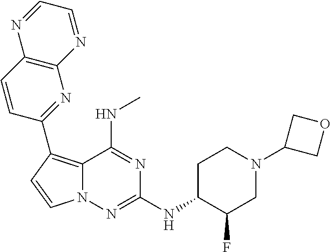 [SMILES: CNc1nc(N[C@@H]2CCN(C3COC3)C[C@H]2F)nn2ccc(-c3ccc4nccnc4n3)c12] |
| 323 | 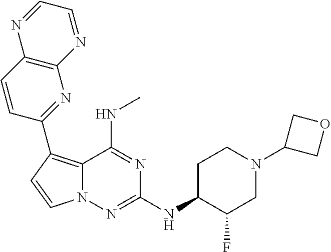 [SMILES: CNc1nc(N[C@H]2CCN(C3COC3)C[C@@H]2F)nn2ccc(-c3ccc4nccnc4n3)c12] |
| 324 | 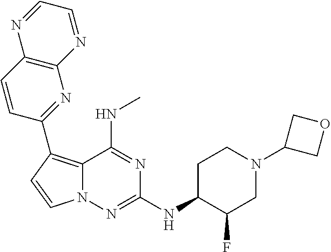 [SMILES: CNc1nc(N[C@H]2CCN(C3COC3)C[C@H]2F)nn2ccc(-c3ccc4nccnc4n3)c12] |
| 325 | 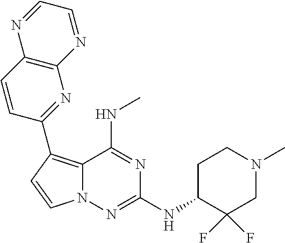 [SMILES: CNc1nc(N[C@@H]2CCN(C)CC2(F)F)nn2ccc(-c3ccc4nccnc4n3)c12] |
| 326 | 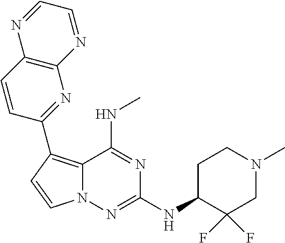 [SMILES: CNc1nc(N[C@H]2CCN(C)CC2(F)F)nn2ccc(-c3ccc4nccnc4n3)c12] |
| 327 | 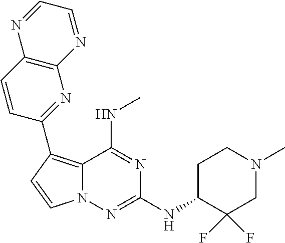 [SMILES: CNc1nc(N[C@@H]2CCN(C)CC2(F)F)nn2ccc(-c3ccc4nccnc4n3)c12] |
| 238 | 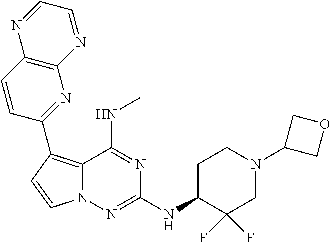 [SMILES: CNc1nc(N[C@H]2CCN(C3COC3)CC2(F)F)nn2ccc(-c3ccc4nccnc4n3)c12] |
| 329 | 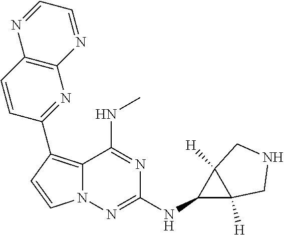 [SMILES: CNc1nc(N[C@@H]2[C@@H]3CNC[C@@H]32)nn2ccc(-c3ccc4nccnc4n3)c12] |
| 330 | 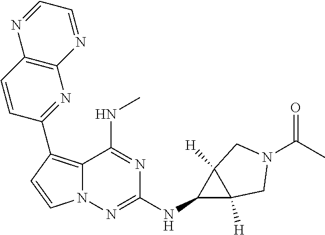 [SMILES: CNc1nc(N[C@@H]2[C@@H]3CN(C(C)=O)C[C@@H]32)nn2ccc(-c3ccc4nccnc4n3)c12] |
| 331 | 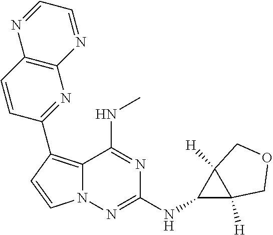 [SMILES: CNc1nc(N[C@H]2[C@@H]3COC[C@@H]32)nn2ccc(-c3ccc4nccnc4n3)c12] |
| 332 | 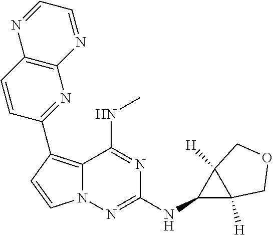 [SMILES: CNc1nc(N[C@@H]2[C@@H]3COC[C@@H]32)nn2ccc(-c3ccc4nccnc4n3)c12] |
| 333 | 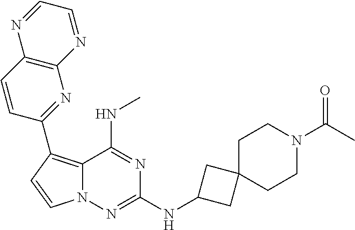 [SMILES: CNc1nc(NC2CC3(CCN(C(C)=O)CC3)C2)nn2ccc(-c3ccc4nccnc4n3)c12] |
| 334 | 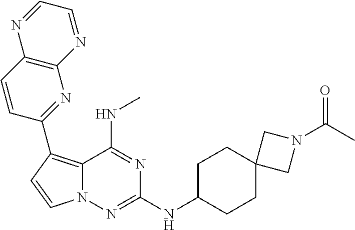 [SMILES: CNc1nc(NC2CCC3(CC2)CN(C(C)=O)C3)nn2ccc(-c3ccc4nccnc4n3)c12] |
| 335 | 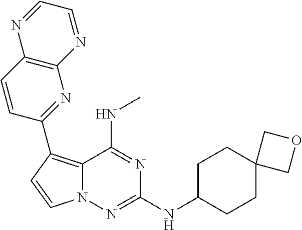 [SMILES: CNc1nc(NC2CCC3(CC2)COC3)nn2ccc(-c3ccc4nccnc4n3)c12] |
| 336 | 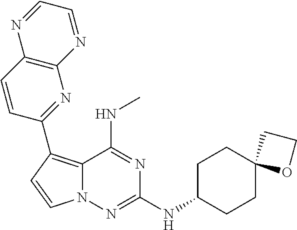 [SMILES: CNc1nc(N[C@H]2CC[C@@]3(CCO3)CC2)nn2ccc(-c3ccc4nccnc4n3)c12] |
| 337 | 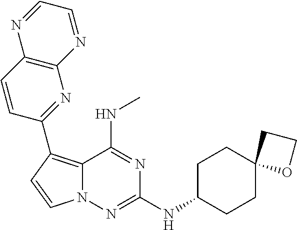 [SMILES: CNc1nc(N[C@H]2CC[C@]3(CCO3)CC2)nn2ccc(-c3ccc4nccnc4n3)c12] |
| 338 | 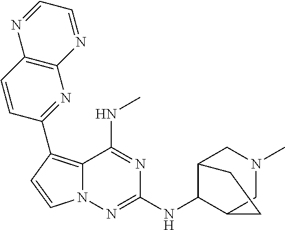 [SMILES: CNc1nc(NC2C3CCC2CN(C)C3)nn2ccc(-c3ccc4nccnc4n3)c12] |
| 339 | 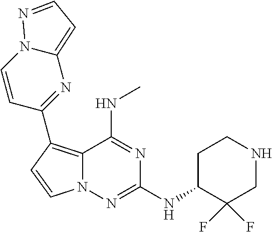 [SMILES: CNc1nc(N[C@@H]2CCNCC2(F)F)nn2ccc(-c3ccn4nccc4n3)c12] |
| 340 | 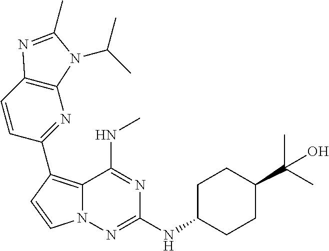 [SMILES: CNc1nc(N[C@H]2CC[C@H](C(C)(C)O)CC2)nn2ccc(-c3ccc4nc(C)n(C(C)C)c4n3)c12] |
| 341 | 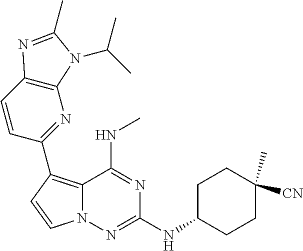 [SMILES: CNc1nc(N[C@H]2CC[C@](C)(C#N)CC2)nn2ccc(-c3ccc4nc(C)n(C(C)C)c4n3)c12] |
| 342 | 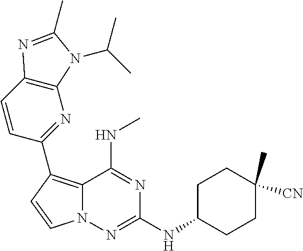 [SMILES: CNc1nc(N[C@H]2CC[C@@](C)(C#N)CC2)nn2ccc(-c3ccc4nc(C)n(C(C)C)c4n3)c12] |
| 343 | 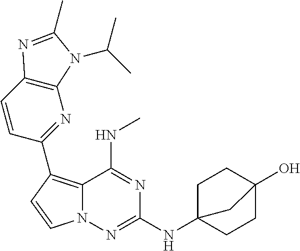 [SMILES: CNc1nc(NC23CCC(O)(CC2)C3)nn2ccc(-c3ccc4nc(C)n(C(C)C)c4n3)c12] |
| 344 | 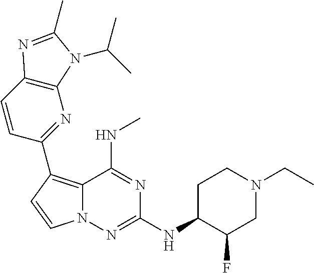 [SMILES: CCN1CC[C@H](Nc2nc(NC)c3c(-c4ccc5nc(C)n(C(C)C)c5n4)ccn3n2)[C@H](F)C1] |
| 345 | 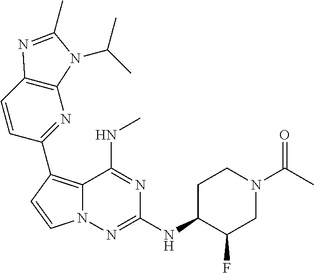 [SMILES: CNc1nc(N[C@H]2CCN(C(C)=O)C[C@H]2F)nn2ccc(-c3ccc4nc(C)n(C(C)C)c4n3)c12] |
| 346 | 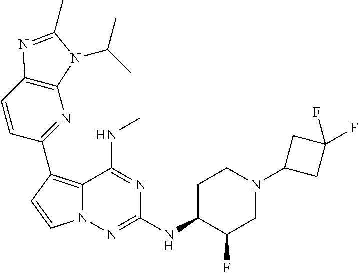 [SMILES: CNc1nc(N[C@H]2CCN(C3CC(F)(F)C3)C[C@H]2F)nn2ccc(-c3ccc4nc(C)n(C(C)C)c4n3)c12] |
| 347 | 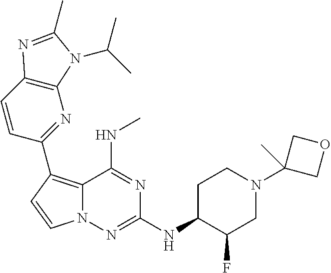 [SMILES: CNc1nc(N[C@H]2CCN(C3(C)COC3)C[C@H]2F)nn2ccc(-c3ccc4nc(C)n(C(C)C)c4n3)c12] |
| 348 | 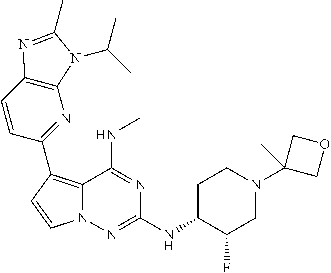 [SMILES: CNc1nc(N[C@@H]2CCN(C3(C)COC3)C[C@@H]2F)nn2ccc(-c3ccc4nc(C)n(C(C)C)c4n3)c12] |
| 349 | 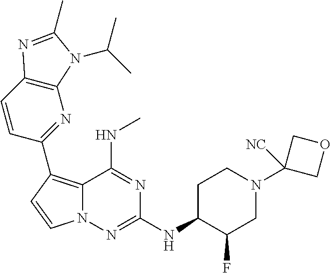 [SMILES: CNc1nc(N[C@H]2CCN(C3(C#N)COC3)C[C@H]2F)nn2ccc(-c3ccc4nc(C)n(C(C)C)c4n3)c12] |
| 350 | 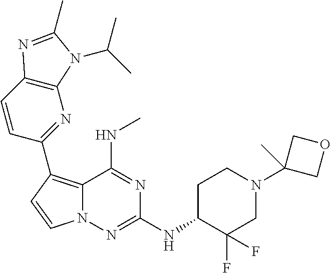 [SMILES: CNc1nc(N[C@@H]2CCN(C3(C)COC3)CC2(F)F)nn2ccc(-c3ccc4nc(C)n(C(C)C)c4n3)c12] |
| 351 | 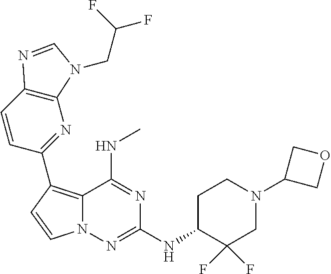 [SMILES: CNc1nc(N[C@@H]2CCN(C3COC3)CC2(F)F)nn2ccc(-c3ccc4ncn(CC(F)F)c4n3)c12] |
| 352 | 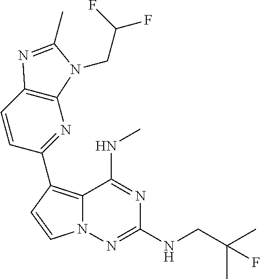 [SMILES: CNc1nc(NCC(C)(C)F)nn2ccc(-c3ccc4nc(C)n(CC(F)F)c4n3)c12] |
| 353 | 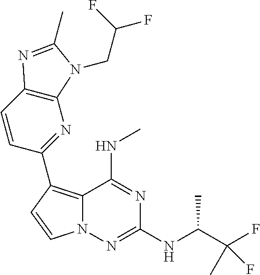 [SMILES: CNc1nc(N[C@H](C)C(C)(F)F)nn2ccc(-c3ccc4nc(C)n(CC(F)F)c4n3)c12] |
| 354 | 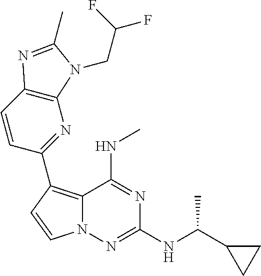 [SMILES: CNc1nc(N[C@H](C)C2CC2)nn2ccc(-c3ccc4nc(C)n(CC(F)F)c4n3)c12] |
| 355 | 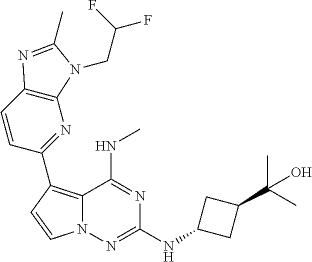 [SMILES: CNc1nc(N[C@H]2C[C@H](C(C)(C)O)C2)nn2ccc(-c3ccc4nc(C)n(CC(F)F)c4n3)c12] |
| 356 | 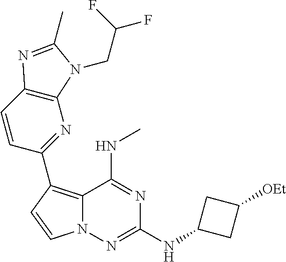 [SMILES: CCO[C@H]1C[C@@H](Nc2nc(NC)c3c(-c4ccc5nc(C)n(CC(F)F)c5n4)ccn3n2)C1] |
| 357 | 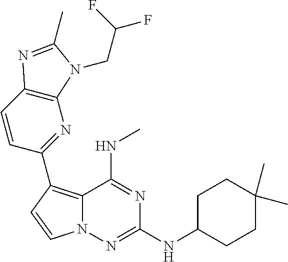 [SMILES: CNc1nc(NC2CCC(C)(C)CC2)nn2ccc(-c3ccc4nc(C)n(CC(F)F)c4n3)c12] |
| 358 | 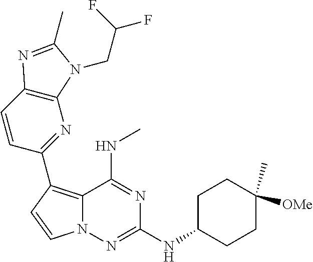 [SMILES: CNc1nc(N[C@H]2CC[C@](C)(OC)CC2)nn2ccc(-c3ccc4nc(C)n(CC(F)F)c4n3)c12] |
| 359 | 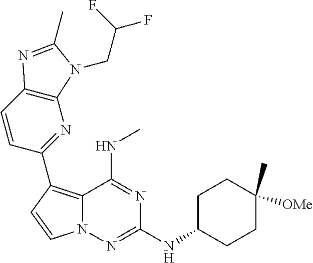 [SMILES: CNc1nc(N[C@H]2CC[C@@](C)(OC)CC2)nn2ccc(-c3ccc4nc(C)n(CC(F)F)c4n3)c12] |
| 360 | 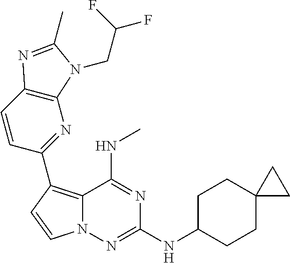 [SMILES: CNc1nc(NC2CCC3(CC2)CC3)nn2ccc(-c3ccc4nc(C)n(CC(F)F)c4n3)c12] |
| 361 | 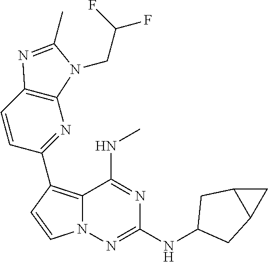 [SMILES: CNc1nc(NC2CC3CC3C2)nn2ccc(-c3ccc4nc(C)n(CC(F)F)c4n3)c12] |
| 362 | 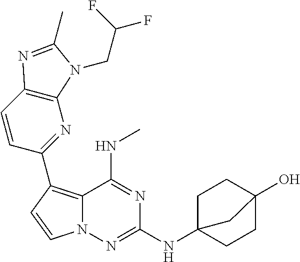 [SMILES: CNc1nc(NC23CCC(O)(CC2)C3)nn2ccc(-c3ccc4nc(C)n(CC(F)F)c4n3)c12] |
| 363 | 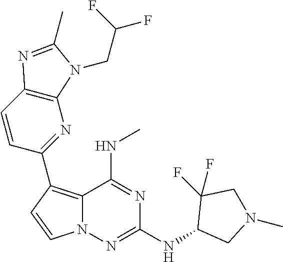 [SMILES: CNc1nc(N[C@H]2CN(C)CC2(F)F)nn2ccc(-c3ccc4nc(C)n(CC(F)F)c4n3)c12] |
| 364 | 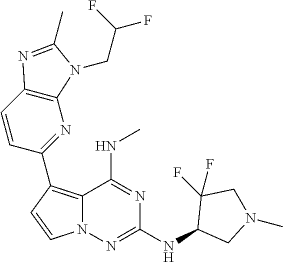 [SMILES: CNc1nc(N[C@@H]2CN(C)CC2(F)F)nn2ccc(-c3ccc4nc(C)n(CC(F)F)c4n3)c12] |
| 365 | 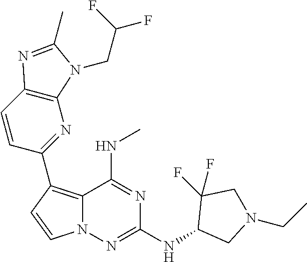 [SMILES: CCN1C[C@H](Nc2nc(NC)c3c(-c4ccc5nc(C)n(CC(F)F)c5n4)ccn3n2)C(F)(F)C1] |
| 366 | 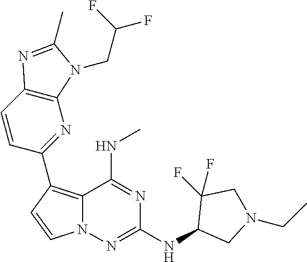 [SMILES: CCN1C[C@@H](Nc2nc(NC)c3c(-c4ccc5nc(C)n(CC(F)F)c5n4)ccn3n2)C(F)(F)C1] |
| 367 | 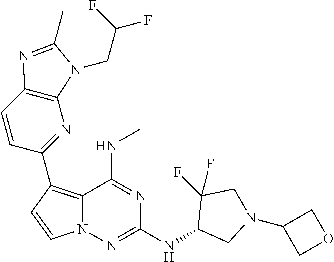 [SMILES: CNc1nc(N[C@H]2CN(C3COC3)CC2(F)F)nn2ccc(-c3ccc4nc(C)n(CC(F)F)c4n3)c12] |
| 368 | 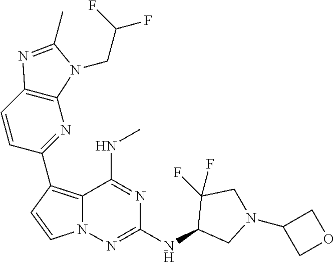 [SMILES: CNc1nc(N[C@@H]2CN(C3COC3)CC2(F)F)nn2ccc(-c3ccc4nc(C)n(CC(F)F)c4n3)c12] |
| 369 | 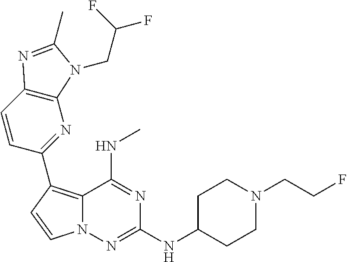 [SMILES: CNc1nc(NC2CCN(CCF)CC2)nn2ccc(-c3ccc4nc(C)n(CC(F)F)c4n3)c12] |
| 370 | 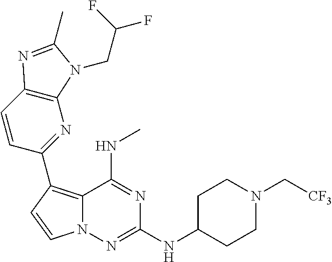 [SMILES: CNc1nc(NC2CCN(CC(F)(F)F)CC2)nn2ccc(-c3ccc4nc(C)n(CC(F)F)c4n3)c12] |
| 371 | 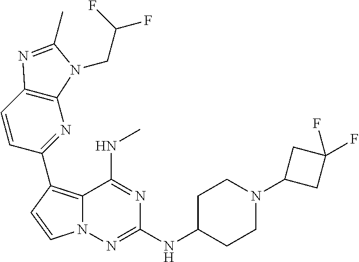 [SMILES: CNc1nc(NC2CCN(C3CC(F)(F)C3)CC2)nn2ccc(-c3ccc4nc(C)n(CC(F)F)c4n3)c12] |
| 372 | 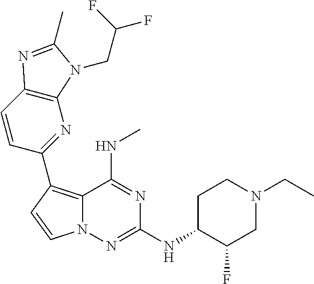 [SMILES: CCN1CC[C@@H](Nc2nc(NC)c3c(-c4ccc5nc(C)n(CC(F)F)c5n4)ccn3n2)[C@@H](F)C1] |
| 373 | 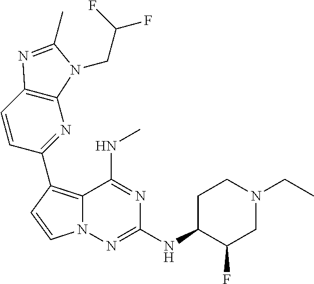 [SMILES: CCN1CC[C@H](Nc2nc(NC)c3c(-c4ccc5nc(C)n(CC(F)F)c5n4)ccn3n2)[C@H](F)C1] |
| 374 | 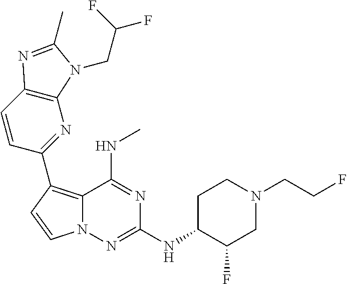 [SMILES: CNc1nc(N[C@@H]2CCN(CCF)C[C@@H]2F)nn2ccc(-c3ccc4nc(C)n(CC(F)F)c4n3)c12] |
| 375 | 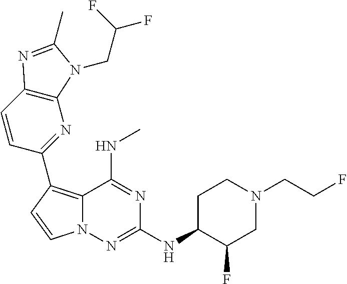 [SMILES: CNc1nc(N[C@H]2CCN(CCF)C[C@H]2F)nn2ccc(-c3ccc4nc(C)n(CC(F)F)c4n3)c12] |
| 376 | 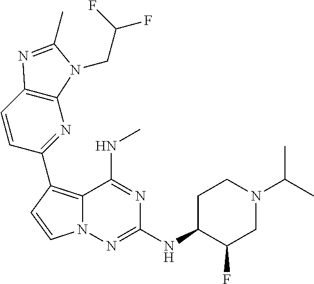 [SMILES: CNc1nc(N[C@H]2CCN(C(C)C)C[C@H]2F)nn2ccc(-c3ccc4nc(C)n(CC(F)F)c4n3)c12] |
| 377 | 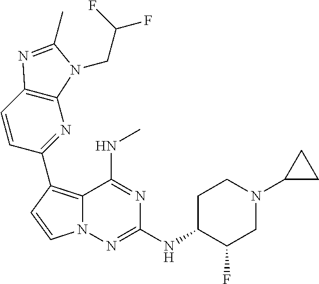 [SMILES: CNc1nc(N[C@@H]2CCN(C3CC3)C[C@@H]2F)nn2ccc(-c3ccc4nc(C)n(CC(F)F)c4n3)c12] |
| 378 | 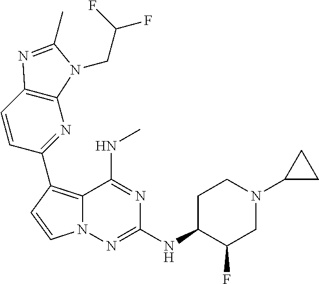 [SMILES: CNc1nc(N[C@H]2CCN(C3CC3)C[C@H]2F)nn2ccc(-c3ccc4nc(C)n(CC(F)F)c4n3)c12] |
| 379 | 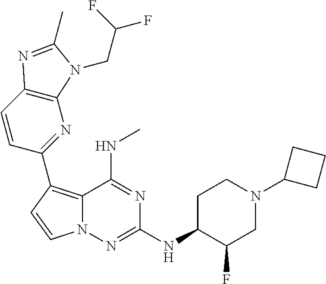 [SMILES: CNc1nc(N[C@H]2CCN(C3CCC3)C[C@H]2F)nn2ccc(-c3ccc4nc(C)n(CC(F)F)c4n3)c12] |
| 380 | 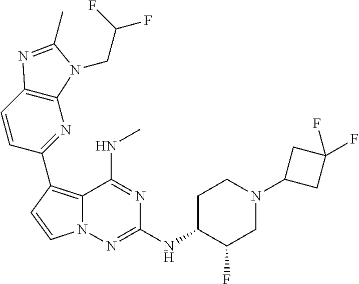 [SMILES: CNc1nc(N[C@@H]2CCN(C3CC(F)(F)C3)C[C@@H]2F)nn2ccc(-c3ccc4nc(C)n(CC(F)F)c4n3)c12] |
| 381 | 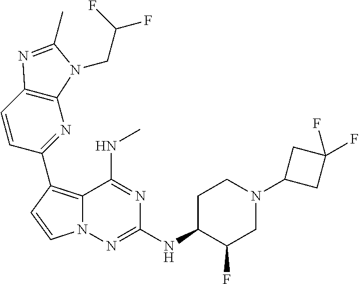 [SMILES: CNc1nc(N[C@H]2CCN(C3CC(F)(F)C3)C[C@H]2F)nn2ccc(-c3ccc4nc(C)n(CC(F)F)c4n3)c12] |
| 382 | 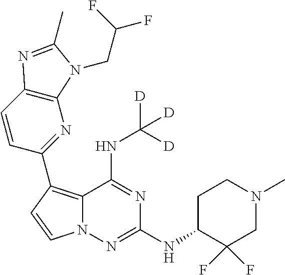 [SMILES: [2H]C([2H])([2H])Nc1nc(N[C@@H]2CCN(C)CC2(F)F)nn2ccc(-c3ccc4nc(C)n(CC(F)F)c4n3)c12] |
| 383 | 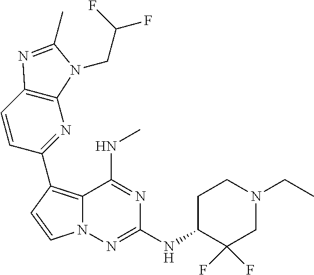 [SMILES: CCN1CC[C@@H](Nc2nc(NC)c3c(-c4ccc5nc(C)n(CC(F)F)c5n4)ccn3n2)C(F)(F)C1] |
| 384 | 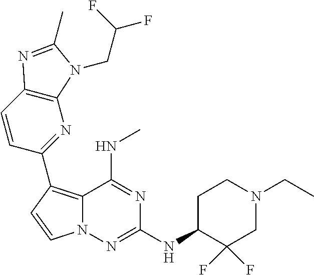 [SMILES: CCN1CC[C@H](Nc2nc(NC)c3c(-c4ccc5nc(C)n(CC(F)F)c5n4)ccn3n2)C(F)(F)C1] |
| 385 | 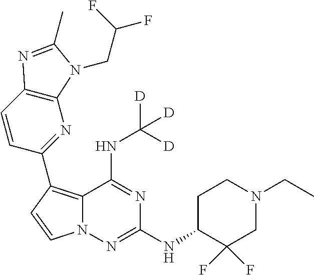 [SMILES: [2H]C([2H])([2H])Nc1nc(N[C@@H]2CCN(CC)CC2(F)F)nn2ccc(-c3ccc4nc(C)n(CC(F)F)c4n3)c12] |
| 386 | 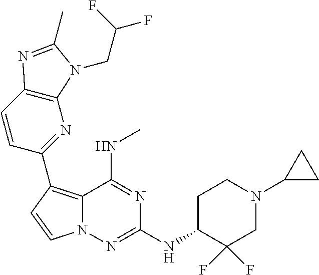 [SMILES: CNc1nc(N[C@@H]2CCN(C3CC3)CC2(F)F)nn2ccc(-c3ccc4nc(C)n(CC(F)F)c4n3)c12] |
| 387 | 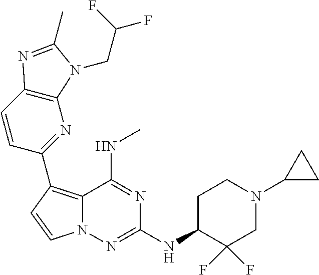 [SMILES: CNc1nc(N[C@H]2CCN(C3CC3)CC2(F)F)nn2ccc(-c3ccc4nc(C)n(CC(F)F)c4n3)c12] |
| 388 | 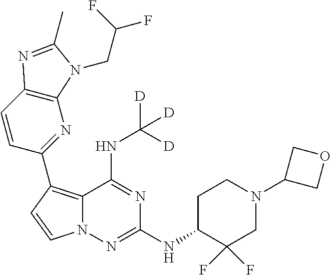 [SMILES: [2H]C([2H])([2H])Nc1nc(N[C@@H]2CCN(C3COC3)CC2(F)F)nn2ccc(-c3ccc4nc(C)n(CC(F)F)c4n3)c12] |
| 389 | 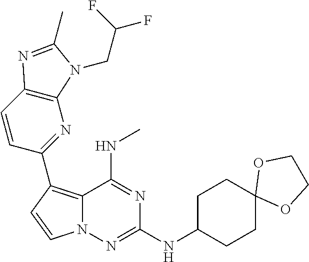 [SMILES: CNc1nc(NC2CCC3(CC2)OCCO3)nn2ccc(-c3ccc4nc(C)n(CC(F)F)c4n3)c12] |
| 390 | 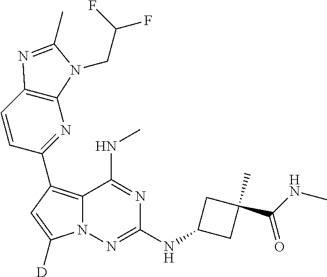 [SMILES: [2H]c1cc(-c2ccc3nc(C)n(CC(F)F)c3n2)c2c(NC)nc(N[C@H]3C[C@](C)(C(=O)NC)C3)nn12] |
| 391 | 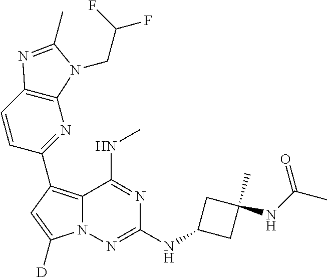 [SMILES: [2H]c1cc(-c2ccc3nc(C)n(CC(F)F)c3n2)c2c(NC)nc(N[C@H]3C[C@](C)(NC(C)=O)C3)nn12] |
| 392 | 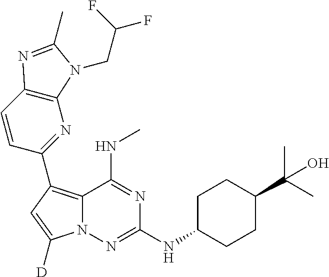 [SMILES: [2H]c1cc(-c2ccc3nc(C)n(CC(F)F)c3n2)c2c(NC)nc(N[C@H]3CC[C@H](C(C)(C)O)CC3)nn12] |
| 393 | 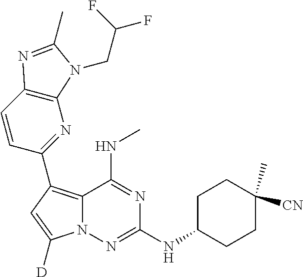 [SMILES: [2H]c1cc(-c2ccc3nc(C)n(CC(F)F)c3n2)c2c(NC)nc(N[C@H]3CC[C@](C)(C#N)CC3)nn12] |
| 394 | 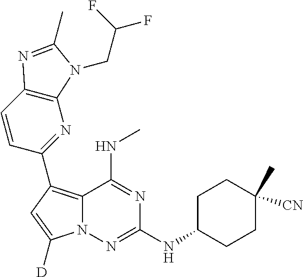 [SMILES: [2H]c1cc(-c2ccc3nc(C)n(CC(F)F)c3n2)c2c(NC)nc(N[C@H]3CC[C@@](C)(C#N)CC3)nn12] |
| 395 | 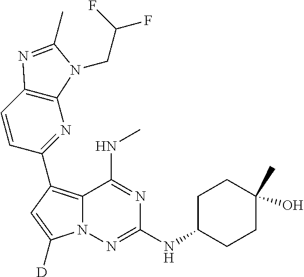 [SMILES: [2H]c1cc(-c2ccc3nc(C)n(CC(F)F)c3n2)c2c(NC)nc(N[C@H]3CC[C@@](C)(O)CC3)nn12] |
| 396 | 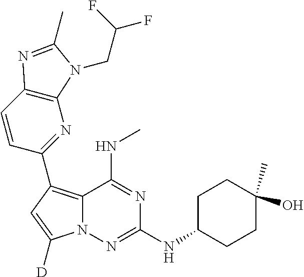 [SMILES: [2H]c1cc(-c2ccc3nc(C)n(CC(F)F)c3n2)c2c(NC)nc(N[C@H]3CC[C@](C)(O)CC3)nn12] |
| 397 | 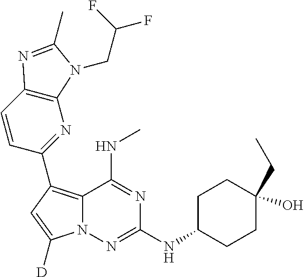 [SMILES: [2H]c1cc(-c2ccc3nc(C)n(CC(F)F)c3n2)c2c(NC)nc(N[C@H]3CC[C@](O)(CC)CC3)nn12] |
| 398 | 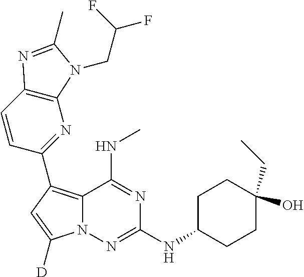 [SMILES: [2H]c1cc(-c2ccc3nc(C)n(CC(F)F)c3n2)c2c(NC)nc(N[C@H]3CC[C@@](O)(CC)CC3)nn12] |
| 399 | 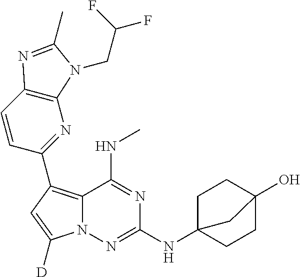 [SMILES: [2H]c1cc(-c2ccc3nc(C)n(CC(F)F)c3n2)c2c(NC)nc(NC34CCC(O)(CC3)C4)nn12] |
| 400 | 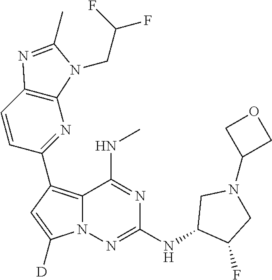 [SMILES: [2H]c1cc(-c2ccc3nc(C)n(CC(F)F)c3n2)c2c(NC)nc(N[C@@H]3CN(C4COC4)C[C@@H]3F)nn12] |
| 401 | 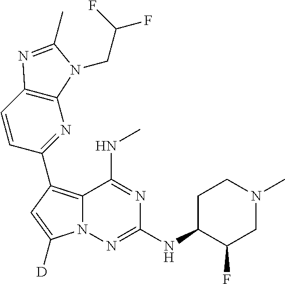 [SMILES: [2H]c1cc(-c2ccc3nc(C)n(CC(F)F)c3n2)c2c(NC)nc(N[C@H]3CCN(C)C[C@H]3F)nn12] |
| 402 | 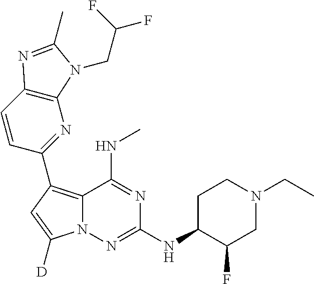 [SMILES: [2H]c1cc(-c2ccc3nc(C)n(CC(F)F)c3n2)c2c(NC)nc(N[C@H]3CCN(CC)C[C@H]3F)nn12] |
| 403 | 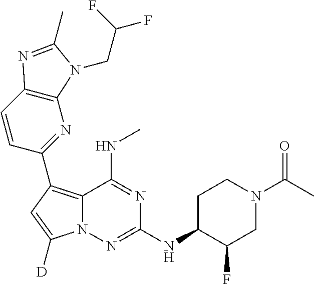 [SMILES: [2H]c1cc(-c2ccc3nc(C)n(CC(F)F)c3n2)c2c(NC)nc(N[C@H]3CCN(C(C)=O)C[C@H]3F)nn12] |
| 404 | 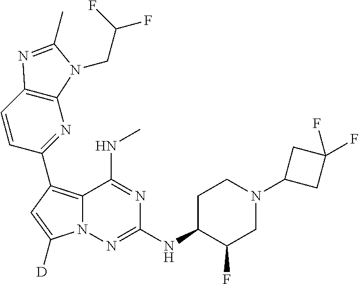 [SMILES: [2H]c1cc(-c2ccc3nc(C)n(CC(F)F)c3n2)c2c(NC)nc(N[C@H]3CCN(C4CC(F)(F)C4)C[C@H]3F)nn12] |
| 405 | 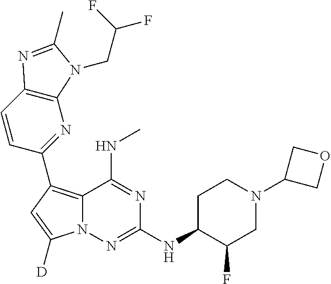 [SMILES: [2H]c1cc(-c2ccc3nc(C)n(CC(F)F)c3n2)c2c(NC)nc(N[C@H]3CCN(C4COC4)C[C@H]3F)nn12] |
| 406 | 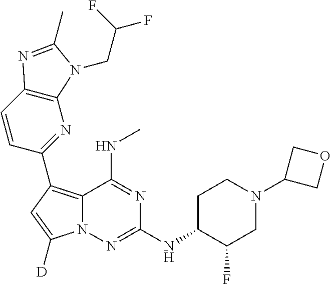 [SMILES: [2H]c1cc(-c2ccc3nc(C)n(CC(F)F)c3n2)c2c(NC)nc(N[C@@H]3CCN(C4COC4)C[C@@H]3F)nn12] |
| 407 | 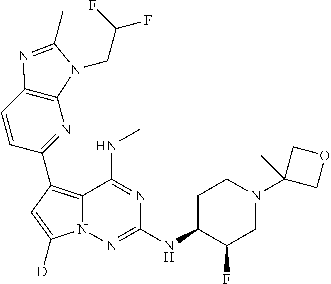 [SMILES: [2H]c1cc(-c2ccc3nc(C)n(CC(F)F)c3n2)c2c(NC)nc(N[C@H]3CCN(C4(C)COC4)C[C@H]3F)nn12] |
| 408 | 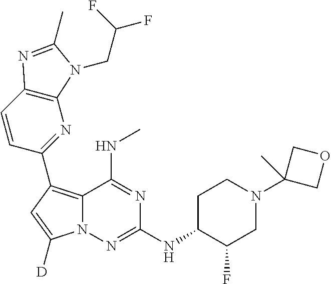 [SMILES: [2H]c1cc(-c2ccc3nc(C)n(CC(F)F)c3n2)c2c(NC)nc(N[C@@H]3CCN(C4(C)COC4)C[C@@H]3F)nn12] |
| 409 | 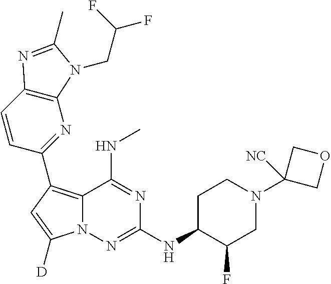 [SMILES: [2H]c1cc(-c2ccc3nc(C)n(CC(F)F)c3n2)c2c(NC)nc(N[C@H]3CCN(C4(C#N)COC4)C[C@H]3F)nn12] |
| 410 | 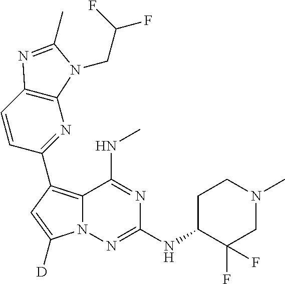 [SMILES: [2H]c1cc(-c2ccc3nc(C)n(CC(F)F)c3n2)c2c(NC)nc(N[C@@H]3CCN(C)CC3(F)F)nn12] |
| 411 | 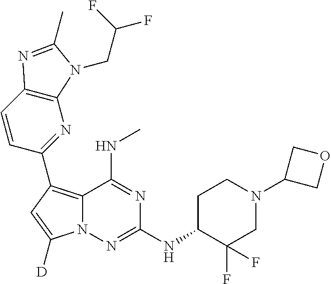 [SMILES: [2H]c1cc(-c2ccc3nc(C)n(CC(F)F)c3n2)c2c(NC)nc(N[C@@H]3CCN(C4COC4)CC3(F)F)nn12] |
| 412 | 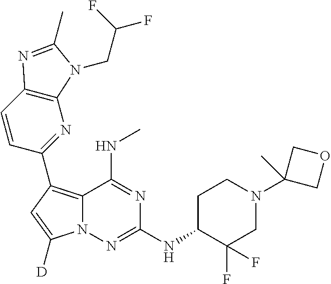 [SMILES: [2H]c1cc(-c2ccc3nc(C)n(CC(F)F)c3n2)c2c(NC)nc(N[C@@H]3CCN(C4(C)COC4)CC3(F)F)nn12] |
| 413 | 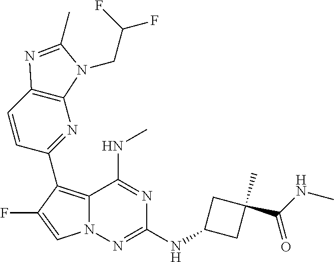 [SMILES: CNc1nc(N[C@H]2C[C@](C)(C(=O)NC)C2)nn2cc(F)c(-c3ccc4nc(C)n(CC(F)F)c4n3)c12] |
| 414 | 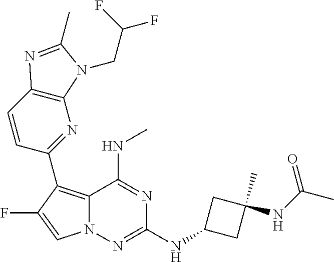 [SMILES: CNc1nc(N[C@H]2C[C@](C)(NC(C)=O)C2)nn2cc(F)c(-c3ccc4nc(C)n(CC(F)F)c4n3)c12] |
| 415 | 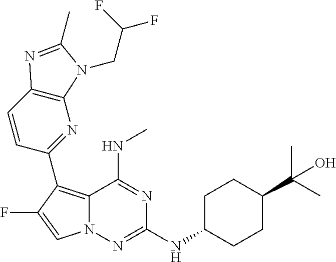 [SMILES: CNc1nc(N[C@H]2CC[C@H](C(C)(C)O)CC2)nn2cc(F)c(-c3ccc4nc(C)n(CC(F)F)c4n3)c12] |
| 416 | 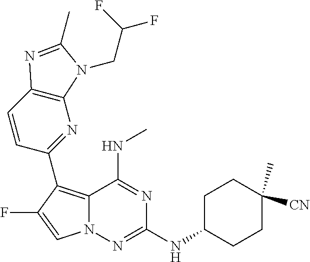 [SMILES: CNc1nc(N[C@H]2CC[C@](C)(C#N)CC2)nn2cc(F)c(-c3ccc4nc(C)n(CC(F)F)c4n3)c12] |
| 417 | 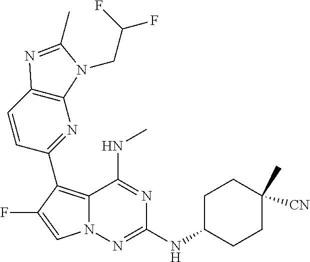 [SMILES: CNc1nc(N[C@H]2CC[C@@](C)(C#N)CC2)nn2cc(F)c(-c3ccc4nc(C)n(CC(F)F)c4n3)c12] |
| 418 | 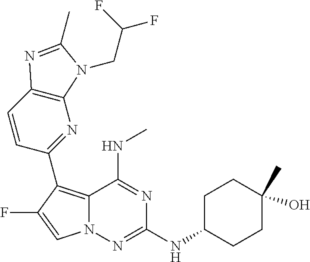 [SMILES: CNc1nc(N[C@H]2CC[C@@](C)(O)CC2)nn2cc(F)c(-c3ccc4nc(C)n(CC(F)F)c4n3)c12] |
| 419 | 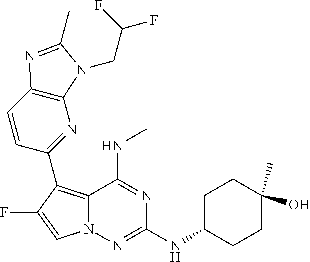 [SMILES: CNc1nc(N[C@H]2CC[C@](C)(O)CC2)nn2cc(F)c(-c3ccc4nc(C)n(CC(F)F)c4n3)c12] |
| 420 | 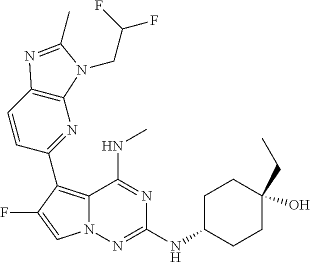 [SMILES: CC[C@]1(O)CC[C@@H](Nc2nc(NC)c3c(-c4ccc5nc(C)n(CC(F)F)c5n4)c(F)cn3n2)CC1] |
| 421 | 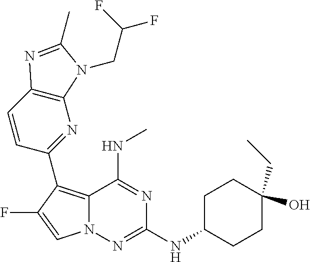 [SMILES: CC[C@]1(O)CC[C@H](Nc2nc(NC)c3c(-c4ccc5nc(C)n(CC(F)F)c5n4)c(F)cn3n2)CC1] |
| 422 | 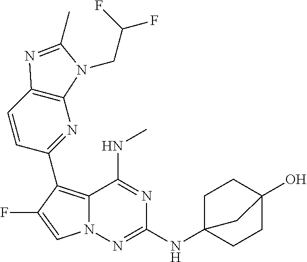 [SMILES: CNc1nc(NC23CCC(O)(CC2)C3)nn2cc(F)c(-c3ccc4nc(C)n(CC(F)F)c4n3)c12] |
| 423 | 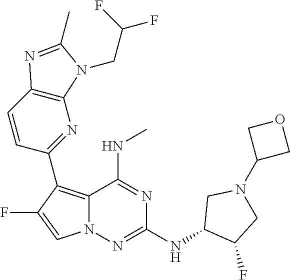 [SMILES: CNc1nc(N[C@@H]2CN(C3COC3)C[C@@H]2F)nn2cc(F)c(-c3ccc4nc(C)n(CC(F)F)c4n3)c12] |
| 424 | 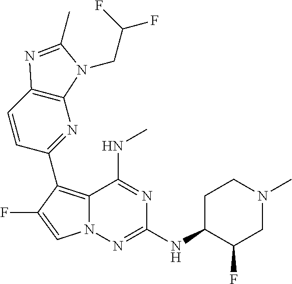 [SMILES: CNc1nc(N[C@H]2CCN(C)C[C@H]2F)nn2cc(F)c(-c3ccc4nc(C)n(CC(F)F)c4n3)c12] |
| 425 | 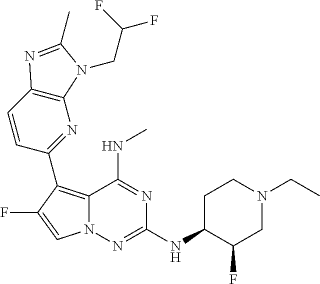 [SMILES: CCN1CC[C@H](Nc2nc(NC)c3c(-c4ccc5nc(C)n(CC(F)F)c5n4)c(F)cn3n2)[C@H](F)C1] |
| 426 | 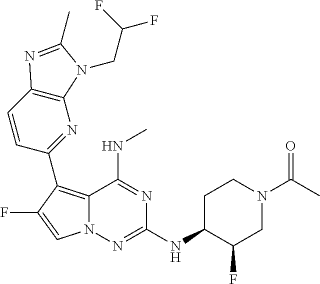 [SMILES: CNc1nc(N[C@H]2CCN(C(C)=O)C[C@H]2F)nn2cc(F)c(-c3ccc4nc(C)n(CC(F)F)c4n3)c12] |
| 427 | 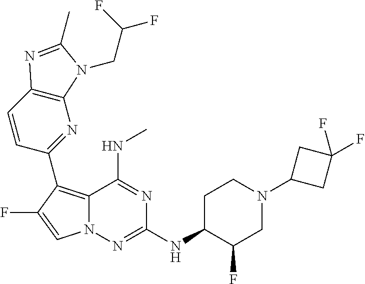 [SMILES: CNc1nc(N[C@H]2CCN(C3CC(F)(F)C3)C[C@H]2F)nn2cc(F)c(-c3ccc4nc(C)n(CC(F)F)c4n3)c12] |
| 428 | 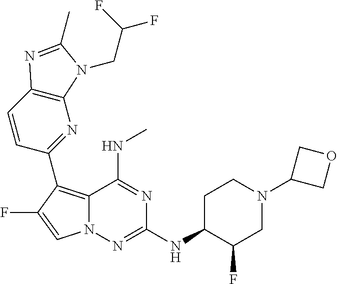 [SMILES: CNc1nc(N[C@H]2CCN(C3COC3)C[C@H]2F)nn2cc(F)c(-c3ccc4nc(C)n(CC(F)F)c4n3)c12] |
| 429 | 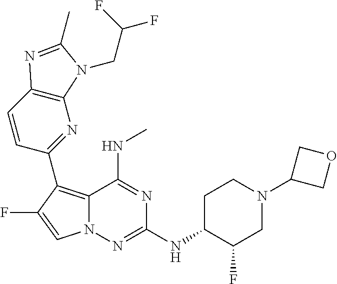 [SMILES: CNc1nc(N[C@@H]2CCN(C3COC3)C[C@@H]2F)nn2cc(F)c(-c3ccc4nc(C)n(CC(F)F)c4n3)c12] |
| 430 | 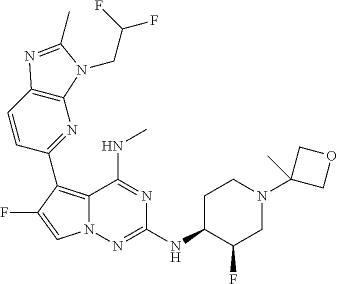 [SMILES: CNc1nc(N[C@H]2CCN(C3(C)COC3)C[C@H]2F)nn2cc(F)c(-c3ccc4nc(C)n(CC(F)F)c4n3)c12] |
| 431 | 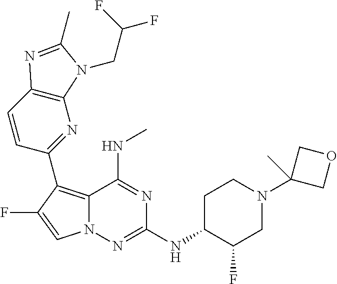 [SMILES: CNc1nc(N[C@@H]2CCN(C3(C)COC3)C[C@@H]2F)nn2cc(F)c(-c3ccc4nc(C)n(CC(F)F)c4n3)c12] |
| 432 | 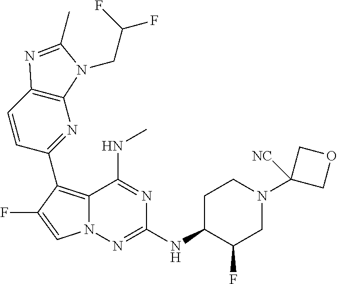 [SMILES: CNc1nc(N[C@H]2CCN(C3(C#N)COC3)C[C@H]2F)nn2cc(F)c(-c3ccc4nc(C)n(CC(F)F)c4n3)c12] |
| 433 | 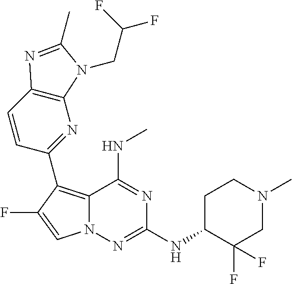 [SMILES: CNc1nc(N[C@@H]2CCN(C)CC2(F)F)nn2cc(F)c(-c3ccc4nc(C)n(CC(F)F)c4n3)c12] |
| 434 | 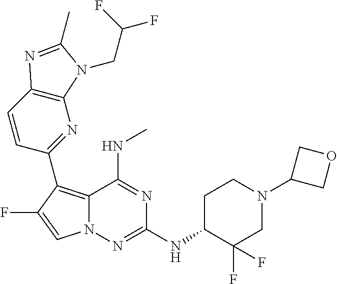 [SMILES: CNc1nc(N[C@@H]2CCN(C3COC3)CC2(F)F)nn2cc(F)c(-c3ccc4nc(C)n(CC(F)F)c4n3)c12] |
| 435 | 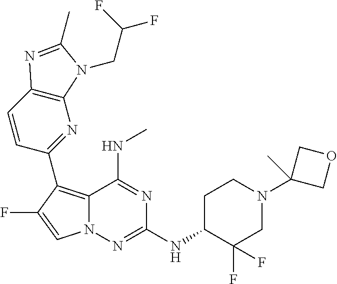 [SMILES: CNc1nc(N[C@@H]2CCN(C3(C)COC3)CC2(F)F)nn2cc(F)c(-c3ccc4nc(C)n(CC(F)F)c4n3)c12] |
| 436 | 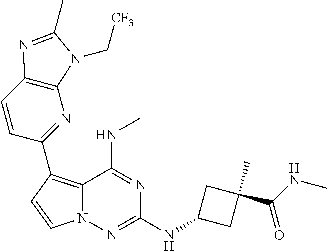 [SMILES: CNc1nc(N[C@H]2C[C@](C)(C(=O)NC)C2)nn2ccc(-c3ccc4nc(C)n(CC(F)(F)F)c4n3)c12] |
| 437 | 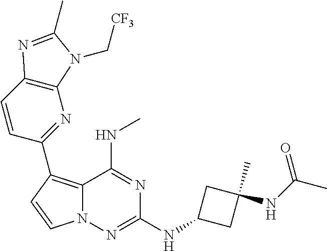 [SMILES: CNc1nc(N[C@H]2C[C@](C)(NC(C)=O)C2)nn2ccc(-c3ccc4nc(C)n(CC(F)(F)F)c4n3)c12] |
| 438 | 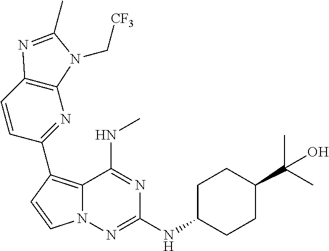 [SMILES: CNc1nc(N[C@H]2CC[C@H](C(C)(C)O)CC2)nn2ccc(-c3ccc4nc(C)n(CC(F)(F)F)c4n3)c12] |
| 439 | 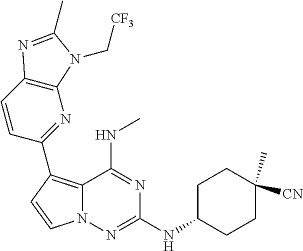 [SMILES: CNc1nc(N[C@H]2CC[C@](C)(C#N)CC2)nn2ccc(-c3ccc4nc(C)n(CC(F)(F)F)c4n3)c12] |
| 440 | 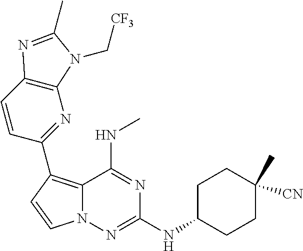 [SMILES: CNc1nc(N[C@H]2CC[C@@](C)(C#N)CC2)nn2ccc(-c3ccc4nc(C)n(CC(F)(F)F)c4n3)c12] |
| 441 | 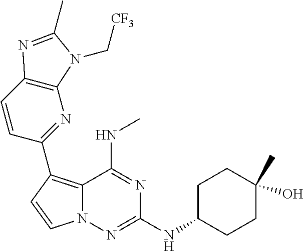 [SMILES: CNc1nc(N[C@H]2CC[C@@](C)(O)CC2)nn2ccc(-c3ccc4nc(C)n(CC(F)(F)F)c4n3)c12] |
| 442 | 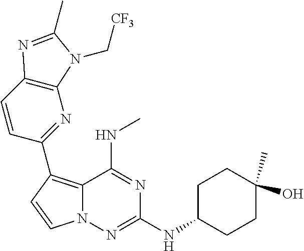 [SMILES: CNc1nc(N[C@H]2CC[C@](C)(O)CC2)nn2ccc(-c3ccc4nc(C)n(CC(F)(F)F)c4n3)c12] |
| 443 | 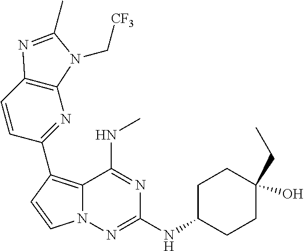 [SMILES: CC[C@]1(O)CC[C@@H](Nc2nc(NC)c3c(-c4ccc5nc(C)n(CC(F)(F)F)c5n4)ccn3n2)CC1] |
| 444 | 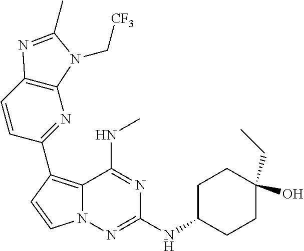 [SMILES: CC[C@]1(O)CC[C@H](Nc2nc(NC)c3c(-c4ccc5nc(C)n(CC(F)(F)F)c5n4)ccn3n2)CC1] |
| 445 | 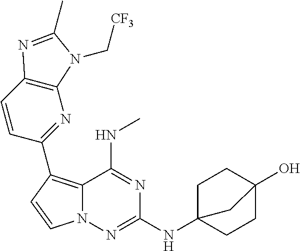 [SMILES: CNc1nc(NC23CCC(O)(CC2)C3)nn2ccc(-c3ccc4nc(C)n(CC(F)(F)F)c4n3)c12] |
| 446 | 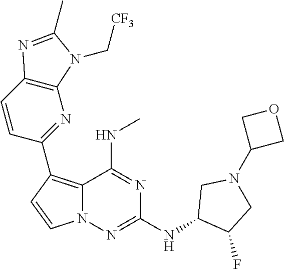 [SMILES: CNc1nc(N[C@@H]2CN(C3COC3)C[C@@H]2F)nn2ccc(-c3ccc4nc(C)n(CC(F)(F)F)c4n3)c12] |
| 447 | 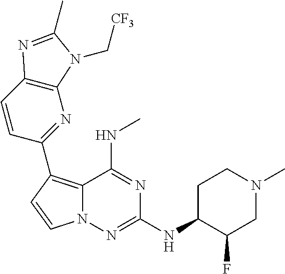 [SMILES: CNc1nc(N[C@H]2CCN(C)C[C@H]2F)nn2ccc(-c3ccc4nc(C)n(CC(F)(F)F)c4n3)c12] |
| 448 | 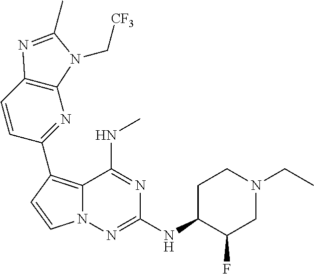 [SMILES: CCN1CC[C@H](Nc2nc(NC)c3c(-c4ccc5nc(C)n(CC(F)(F)F)c5n4)ccn3n2)[C@H](F)C1] |
| 449 | 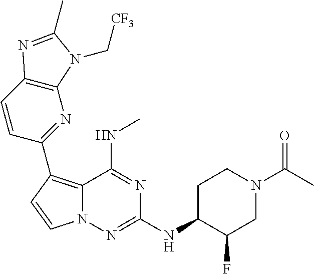 [SMILES: CNc1nc(N[C@H]2CCN(C(C)=O)C[C@H]2F)nn2ccc(-c3ccc4nc(C)n(CC(F)(F)F)c4n3)c12] |
| 450 | 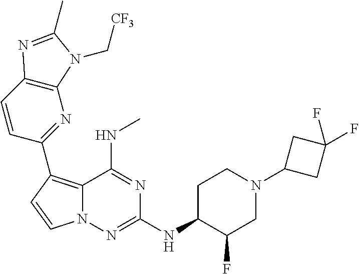 [SMILES: CNc1nc(N[C@H]2CCN(C3CC(F)(F)C3)C[C@H]2F)nn2ccc(-c3ccc4nc(C)n(CC(F)(F)F)c4n3)c12] |
| 451 | 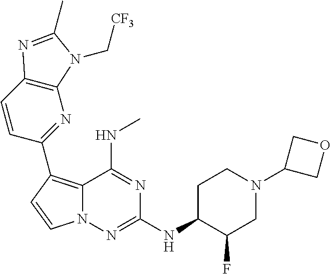 [SMILES: CNc1nc(N[C@H]2CCN(C3COC3)C[C@H]2F)nn2ccc(-c3ccc4nc(C)n(CC(F)(F)F)c4n3)c12] |
| 452 | 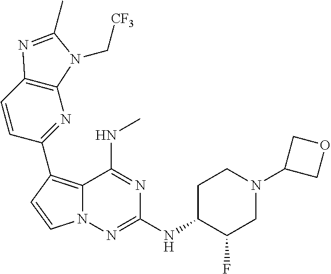 [SMILES: CNc1nc(N[C@@H]2CCN(C3COC3)C[C@@H]2F)nn2ccc(-c3ccc4nc(C)n(CC(F)(F)F)c4n3)c12] |
| 453 | 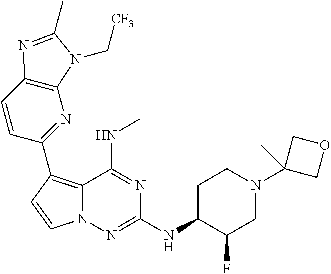 [SMILES: CNc1nc(N[C@H]2CCN(C3(C)COC3)C[C@H]2F)nn2ccc(-c3ccc4nc(C)n(CC(F)(F)F)c4n3)c12] |
| 454 | 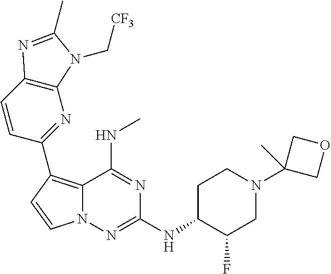 [SMILES: CNc1nc(N[C@@H]2CCN(C3(C)COC3)C[C@@H]2F)nn2ccc(-c3ccc4nc(C)n(CC(F)(F)F)c4n3)c12] |
| 455 | 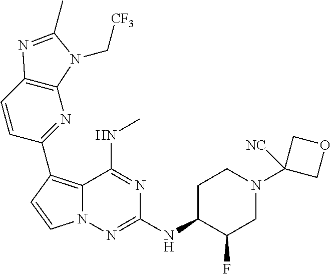 [SMILES: CNc1nc(N[C@H]2CCN(C3(C#N)COC3)C[C@H]2F)nn2ccc(-c3ccc4nc(C)n(CC(F)(F)F)c4n3)c12] |
| 456 | 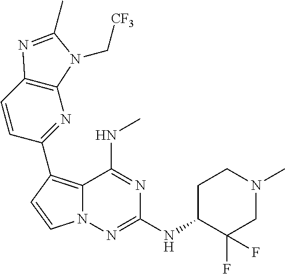 [SMILES: CNc1nc(N[C@@H]2CCN(C)CC2(F)F)nn2ccc(-c3ccc4nc(C)n(CC(F)(F)F)c4n3)c12] |
| 457 | 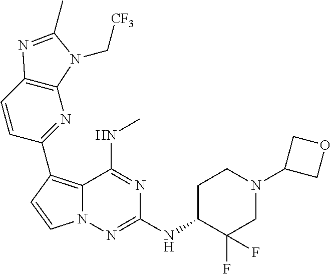 [SMILES: CNc1nc(N[C@@H]2CCN(C3COC3)CC2(F)F)nn2ccc(-c3ccc4nc(C)n(CC(F)(F)F)c4n3)c12] |
| 458 | 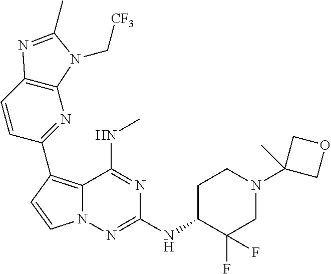 [SMILES: CNc1nc(N[C@@H]2CCN(C3(C)COC3)CC2(F)F)nn2ccc(-c3ccc4nc(C)n(CC(F)(F)F)c4n3)c12] |
| 459 | 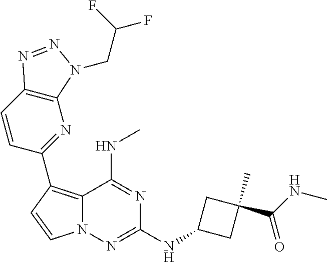 [SMILES: CNc1nc(N[C@H]2C[C@](C)(C(=O)NC)C2)nn2ccc(-c3ccc4nnn(CC(F)F)c4n3)c12] |
| 460 | 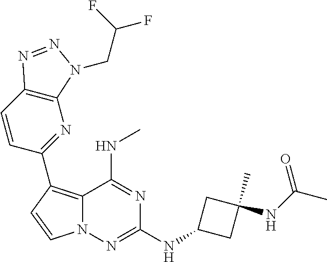 [SMILES: CNc1nc(N[C@H]2C[C@](C)(NC(C)=O)C2)nn2ccc(-c3ccc4nnn(CC(F)F)c4n3)c12] |
| 461 | 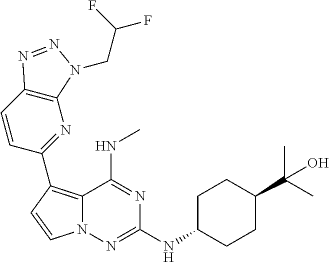 [SMILES: CNc1nc(N[C@H]2CC[C@H](C(C)(C)O)CC2)nn2ccc(-c3ccc4nnn(CC(F)F)c4n3)c12] |
| 462 | 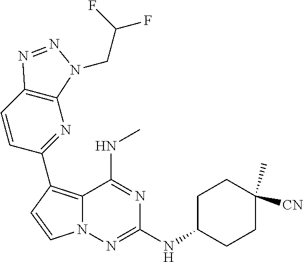 [SMILES: CNc1nc(N[C@H]2CC[C@](C)(C#N)CC2)nn2ccc(-c3ccc4nnn(CC(F)F)c4n3)c12] |
| 463 | 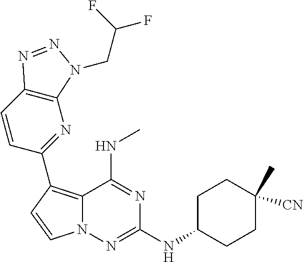 [SMILES: CNc1nc(N[C@H]2CC[C@@](C)(C#N)CC2)nn2ccc(-c3ccc4nnn(CC(F)F)c4n3)c12] |
| 464 | 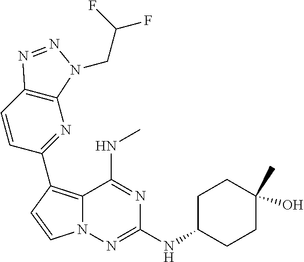 [SMILES: CNc1nc(N[C@H]2CC[C@@](C)(O)CC2)nn2ccc(-c3ccc4nnn(CC(F)F)c4n3)c12] |
| 465 | 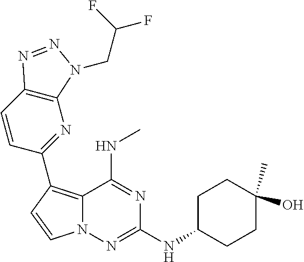 [SMILES: CNc1nc(N[C@H]2CC[C@](C)(O)CC2)nn2ccc(-c3ccc4nnn(CC(F)F)c4n3)c12] |
| 466 | 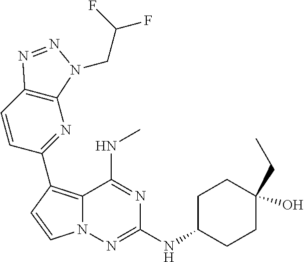 [SMILES: CC[C@]1(O)CC[C@@H](Nc2nc(NC)c3c(-c4ccc5nnn(CC(F)F)c5n4)ccn3n2)CC1] |
| 467 | 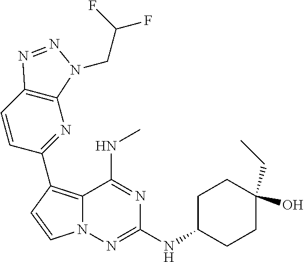 [SMILES: CC[C@]1(O)CC[C@H](Nc2nc(NC)c3c(-c4ccc5nnn(CC(F)F)c5n4)ccn3n2)CC1] |
| 468 | 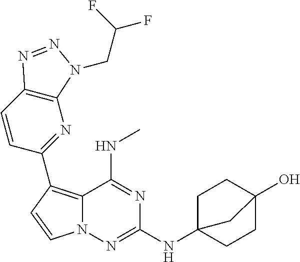 [SMILES: CNc1nc(NC23CCC(O)(CC2)C3)nn2ccc(-c3ccc4nnn(CC(F)F)c4n3)c12] |
| 469 | 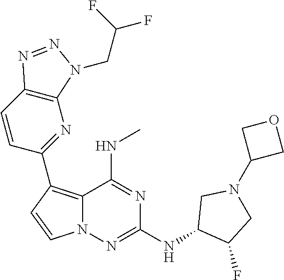 [SMILES: CNc1nc(N[C@@H]2CN(C3COC3)C[C@@H]2F)nn2ccc(-c3ccc4nnn(CC(F)F)c4n3)c12] |
| 470 | 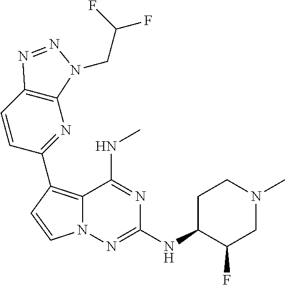 [SMILES: CNc1nc(N[C@H]2CCN(C)C[C@H]2F)nn2ccc(-c3ccc4nnn(CC(F)F)c4n3)c12] |
| 471 | 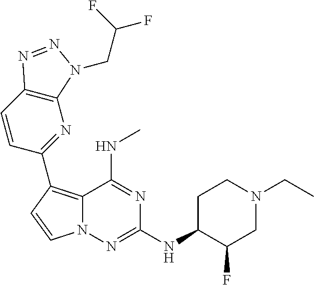 [SMILES: CCN1CC[C@H](Nc2nc(NC)c3c(-c4ccc5nnn(CC(F)F)c5n4)ccn3n2)[C@H](F)C1] |
| 472 | 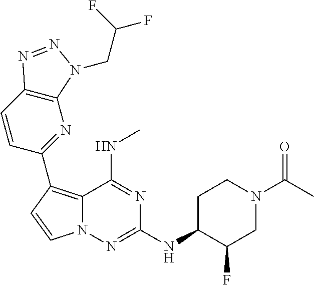 [SMILES: CNc1nc(N[C@H]2CCN(C(C)=O)C[C@H]2F)nn2ccc(-c3ccc4nnn(CC(F)F)c4n3)c12] |
| 473 | 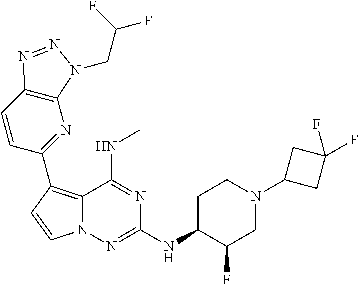 [SMILES: CNc1nc(N[C@H]2CCN(C3CC(F)(F)C3)C[C@H]2F)nn2ccc(-c3ccc4nnn(CC(F)F)c4n3)c12] |
| 474 | 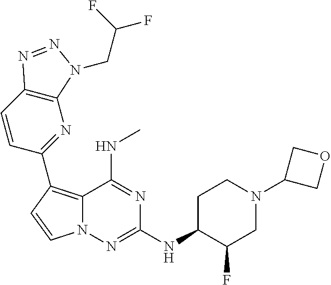 [SMILES: CNc1nc(N[C@H]2CCN(C3COC3)C[C@H]2F)nn2ccc(-c3ccc4nnn(CC(F)F)c4n3)c12] |
| 475 | 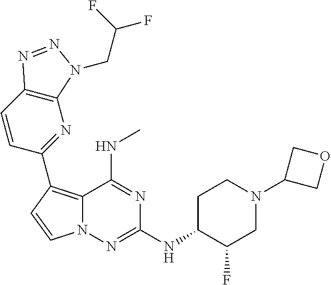 [SMILES: CNc1nc(N[C@@H]2CCN(C3COC3)C[C@@H]2F)nn2ccc(-c3ccc4nnn(CC(F)F)c4n3)c12] |
| 476 | 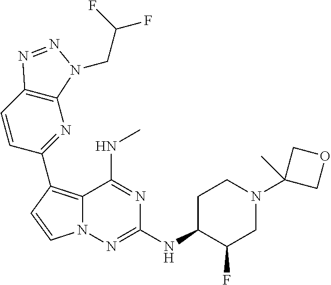 [SMILES: CNc1nc(N[C@H]2CCN(C3(C)COC3)C[C@H]2F)nn2ccc(-c3ccc4nnn(CC(F)F)c4n3)c12] |
| 477 | 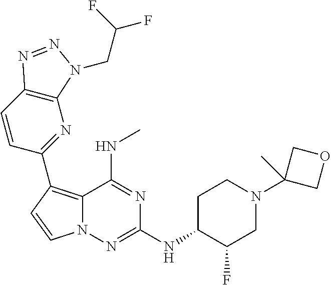 [SMILES: CNc1nc(N[C@@H]2CCN(C3(C)COC3)C[C@@H]2F)nn2ccc(-c3ccc4nnn(CC(F)F)c4n3)c12] |
| 478 | 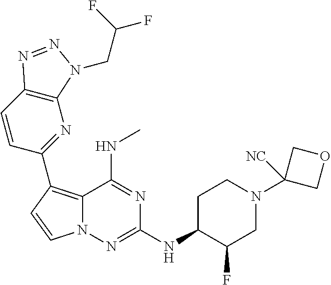 [SMILES: CNc1nc(N[C@H]2CCN(C3(C#N)COC3)C[C@H]2F)nn2ccc(-c3ccc4nnn(CC(F)F)c4n3)c12] |
| 479 | 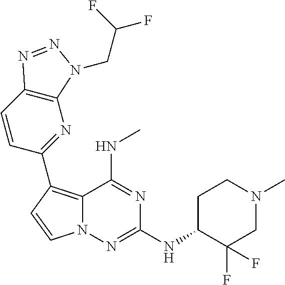 [SMILES: CNc1nc(N[C@@H]2CCN(C)CC2(F)F)nn2ccc(-c3ccc4nnn(CC(F)F)c4n3)c12] |
| 480 | 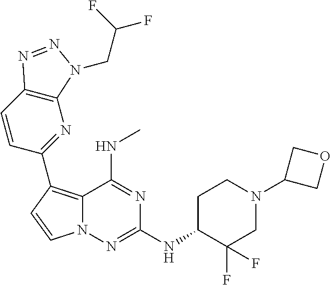 [SMILES: CNc1nc(N[C@@H]2CCN(C3COC3)CC2(F)F)nn2ccc(-c3ccc4nnn(CC(F)F)c4n3)c12] |
| 481 | 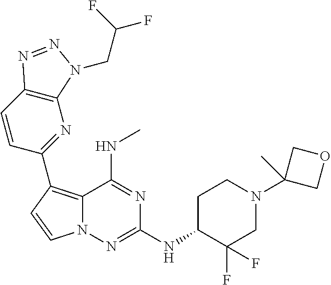 [SMILES: CNc1nc(N[C@@H]2CCN(C3(C)COC3)CC2(F)F)nn2ccc(-c3ccc4nnn(CC(F)F)c4n3)c12] |
| 482 | 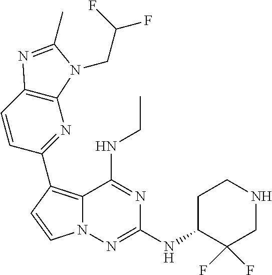 [SMILES: CCNc1nc(N[C@@H]2CCNCC2(F)F)nn2ccc(-c3ccc4nc(C)n(CC(F)F)c4n3)c12] |
| 483 | 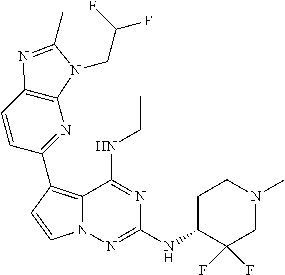 [SMILES: CCNc1nc(N[C@@H]2CCN(C)CC2(F)F)nn2ccc(-c3ccc4nc(C)n(CC(F)F)c4n3)c12] |
| 484 | 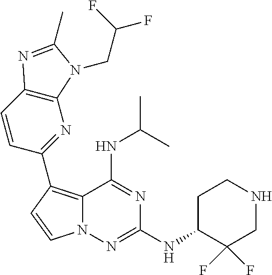 [SMILES: Cc1nc2ccc(-c3ccn4nc(N[C@@H]5CCNCC5(F)F)nc(NC(C)C)c34)nc2n1CC(F)F] |
| 485 | 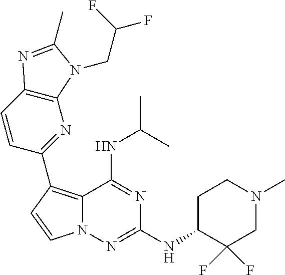 [SMILES: Cc1nc2ccc(-c3ccn4nc(N[C@@H]5CCN(C)CC5(F)F)nc(NC(C)C)c34)nc2n1CC(F)F] |
| 486 | 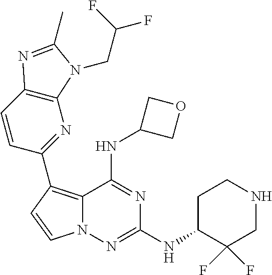 [SMILES: Cc1nc2ccc(-c3ccn4nc(N[C@@H]5CCNCC5(F)F)nc(NC5COC5)c34)nc2n1CC(F)F] |
| 487 | 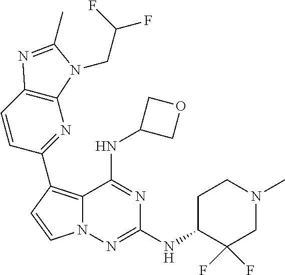 [SMILES: Cc1nc2ccc(-c3ccn4nc(N[C@@H]5CCN(C)CC5(F)F)nc(NC5COC5)c34)nc2n1CC(F)F] |
| 488 | 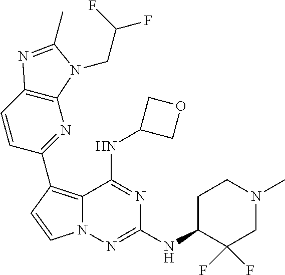 [SMILES: Cc1nc2ccc(-c3ccn4nc(N[C@H]5CCN(C)CC5(F)F)nc(NC5COC5)c34)nc2n1CC(F)F] |
| 489 | 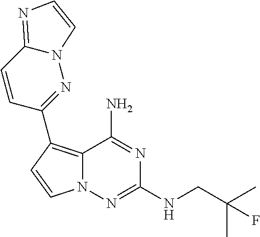 [SMILES: CC(C)(F)CNc1nc(N)c2c(-c3ccc4nccn4n3)ccn2n1] |
| 490 | 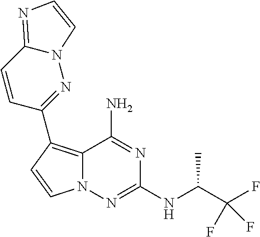 [SMILES: C[C@@H](Nc1nc(N)c2c(-c3ccc4nccn4n3)ccn2n1)C(F)(F)F] |
| 491 | 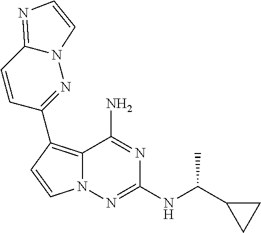 [SMILES: C[C@@H](Nc1nc(N)c2c(-c3ccc4nccn4n3)ccn2n1)C1CC1] |
| 492 | 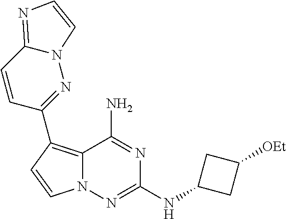 [SMILES: CCO[C@H]1C[C@@H](Nc2nc(N)c3c(-c4ccc5nccn5n4)ccn3n2)C1] |
| 493 | 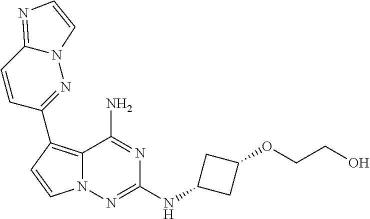 [SMILES: Nc1nc(N[C@H]2C[C@@H](OCCO)C2)nn2ccc(-c3ccc4nccn4n3)c12] |
| 494 | 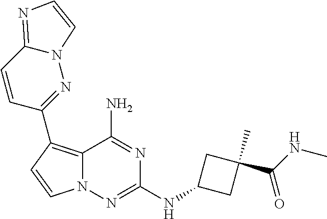 [SMILES: CNC(=O)[C@]1(C)C[C@@H](Nc2nc(N)c3c(-c4ccc5nccn5n4)ccn3n2)C1] |
| 495 | 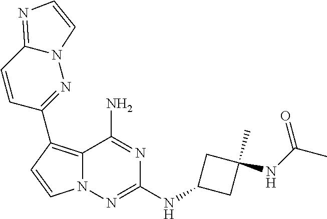 [SMILES: CC(=O)N[C@]1(C)C[C@@H](Nc2nc(N)c3c(-c4ccc5nccn5n4)ccn3n2)C1] |
| 496 | 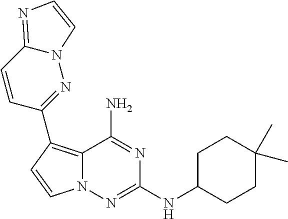 [SMILES: CC1(C)CCC(Nc2nc(N)c3c(-c4ccc5nccn5n4)ccn3n2)CC1] |
| 497 | 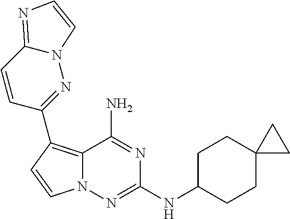 [SMILES: Nc1nc(NC2CCC3(CC2)CC3)nn2ccc(-c3ccc4nccn4n3)c12] |
| 498 | 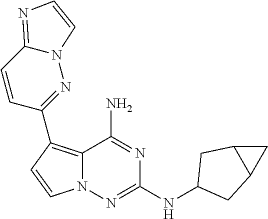 [SMILES: Nc1nc(NC2CC3CC3C2)nn2ccc(-c3ccc4nccn4n3)c12] |
| 499 | 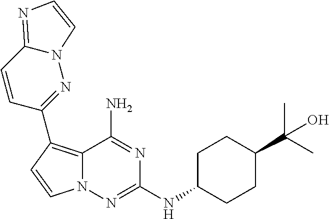 [SMILES: CC(C)(O)[C@H]1CC[C@H](Nc2nc(N)c3c(-c4ccc5nccn5n4)ccn3n2)CC1] |
| 500 | 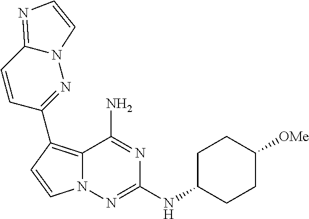 [SMILES: CO[C@H]1CC[C@@H](Nc2nc(N)c3c(-c4ccc5nccn5n4)ccn3n2)CC1] |
| 501 | 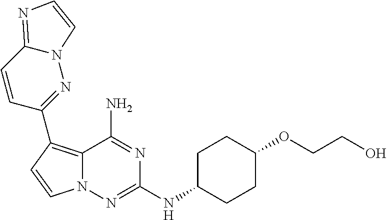 [SMILES: Nc1nc(N[C@H]2CC[C@@H](OCCO)CC2)nn2ccc(-c3ccc4nccn4n3)c12] |
| 502 | 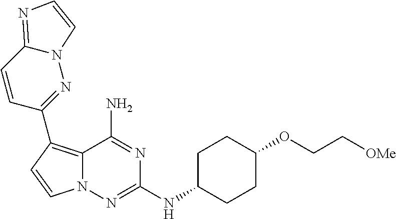 [SMILES: COCCO[C@H]1CC[C@@H](Nc2nc(N)c3c(-c4ccc5nccn5n4)ccn3n2)CC1] |
| 503 | 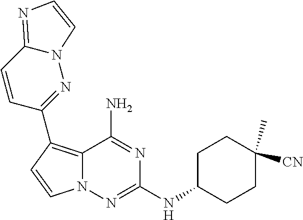 [SMILES: C[C@]1(C#N)CC[C@H](Nc2nc(N)c3c(-c4ccc5nccn5n4)ccn3n2)CC1] |
| 504 | 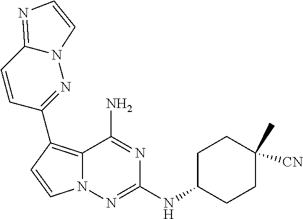 [SMILES: C[C@]1(C#N)CC[C@@H](Nc2nc(N)c3c(-c4ccc5nccn5n4)ccn3n2)CC1] |
| 505 | 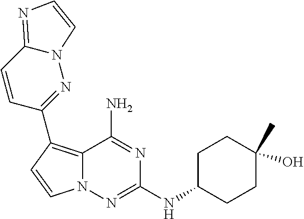 [SMILES: C[C@]1(O)CC[C@@H](Nc2nc(N)c3c(-c4ccc5nccn5n4)ccn3n2)CC1] |
| 506 | 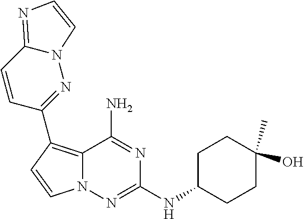 [SMILES: C[C@]1(O)CC[C@H](Nc2nc(N)c3c(-c4ccc5nccn5n4)ccn3n2)CC1] |
| 507 | 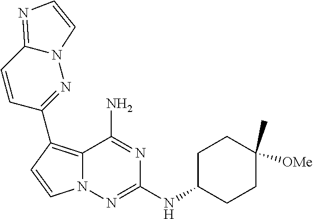 [SMILES: CO[C@]1(C)CC[C@H](Nc2nc(N)c3c(-c4ccc5nccn5n4)ccn3n2)CC1] |
| 508 | 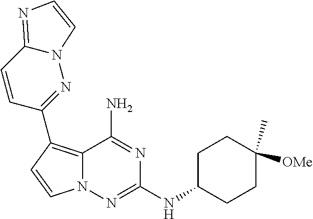 [SMILES: CO[C@]1(C)CC[C@@H](Nc2nc(N)c3c(-c4ccc5nccn5n4)ccn3n2)CC1] |
| 509 | 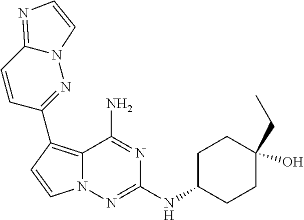 [SMILES: CC[C@]1(O)CC[C@@H](Nc2nc(N)c3c(-c4ccc5nccn5n4)ccn3n2)CC1] |
| 510 | 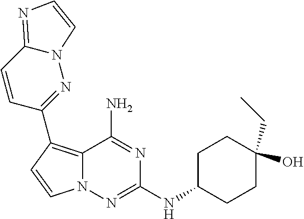 [SMILES: CC[C@]1(O)CC[C@H](Nc2nc(N)c3c(-c4ccc5nccn5n4)ccn3n2)CC1] |
| 511 | 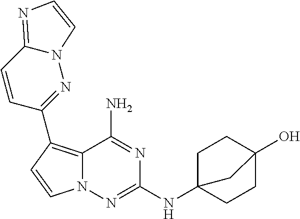 [SMILES: Nc1nc(NC23CCC(O)(CC2)C3)nn2ccc(-c3ccc4nccn4n3)c12] |
| 512 | 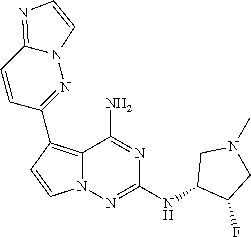 [SMILES: CN1C[C@H](F)[C@H](Nc2nc(N)c3c(-c4ccc5nccn5n4)ccn3n2)C1] |
| 513 | 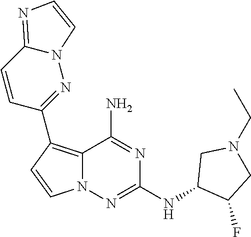 [SMILES: CCN1C[C@H](F)[C@H](Nc2nc(N)c3c(-c4ccc5nccn5n4)ccn3n2)C1] |
| 514 | 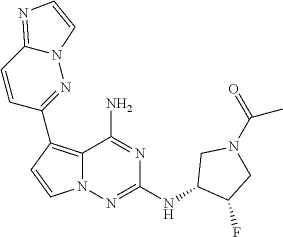 [SMILES: CC(=O)N1C[C@H](F)[C@H](Nc2nc(N)c3c(-c4ccc5nccn5n4)ccn3n2)C1] |
| 515 | 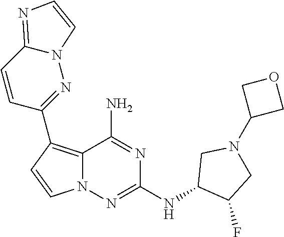 [SMILES: Nc1nc(N[C@@H]2CN(C3COC3)C[C@@H]2F)nn2ccc(-c3ccc4nccn4n3)c12] |
| 516 | 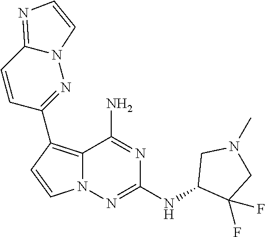 [SMILES: CN1C[C@@H](Nc2nc(N)c3c(-c4ccc5nccn5n4)ccn3n2)C(F)(F)C1] |
| 517 | 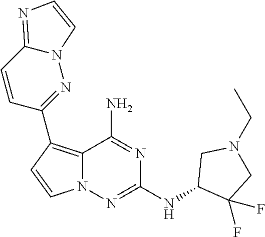 [SMILES: CCN1C[C@@H](Nc2nc(N)c3c(-c4ccc5nccn5n4)ccn3n2)C(F)(F)C1] |
| 518 | 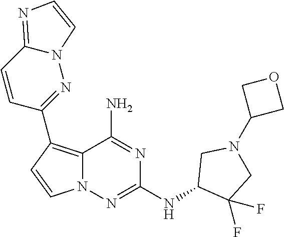 [SMILES: Nc1nc(N[C@@H]2CN(C3COC3)CC2(F)F)nn2ccc(-c3ccc4nccn4n3)c12] |
| 519 | 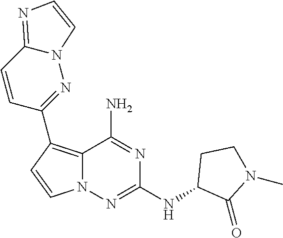 [SMILES: CN1CC[C@@H](Nc2nc(N)c3c(-c4ccc5nccn5n4)ccn3n2)C1=O] |
| 520 | 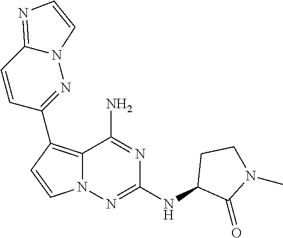 [SMILES: CN1CC[C@H](Nc2nc(N)c3c(-c4ccc5nccn5n4)ccn3n2)C1=O] |
| 521 | 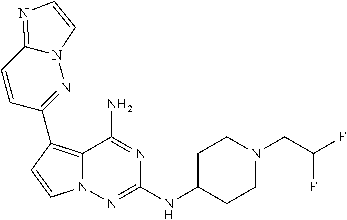 [SMILES: Nc1nc(NC2CCN(CC(F)F)CC2)nn2ccc(-c3ccc4nccn4n3)c12] |
| 522 | 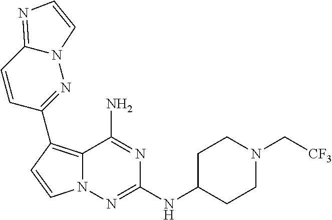 [SMILES: Nc1nc(NC2CCN(CC(F)(F)F)CC2)nn2ccc(-c3ccc4nccn4n3)c12] |
| 523 | 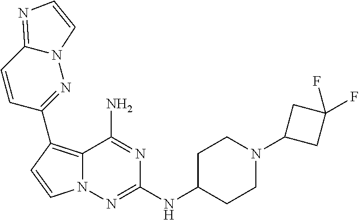 [SMILES: Nc1nc(NC2CCN(C3CC(F)(F)C3)CC2)nn2ccc(-c3ccc4nccn4n3)c12] |
| 524 | 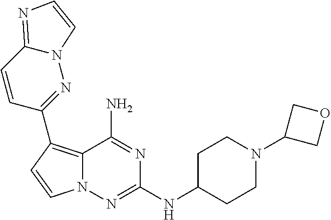 [SMILES: Nc1nc(NC2CCN(C3COC3)CC2)nn2ccc(-c3ccc4nccn4n3)c12] |
| 525 | 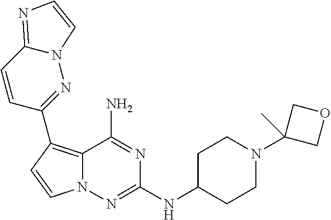 [SMILES: CC1(N2CCC(Nc3nc(N)c4c(-c5ccc6nccn6n5)ccn4n3)CC2)COC1] |
| 526 | 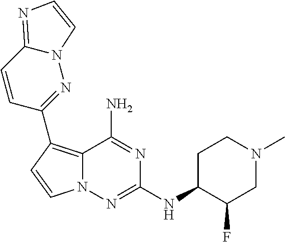 [SMILES: CN1CC[C@H](Nc2nc(N)c3c(-c4ccc5nccn5n4)ccn3n2)[C@H](F)C1] |
| 527 | 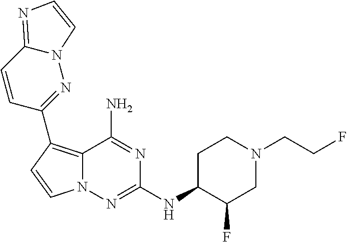 [SMILES: Nc1nc(N[C@H]2CCN(CCF)C[C@H]2F)nn2ccc(-c3ccc4nccn4n3)c12] |
| 528 | 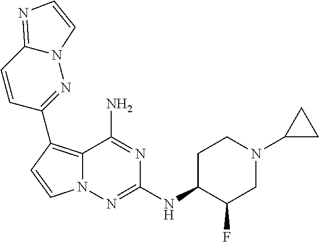 [SMILES: Nc1nc(N[C@H]2CCN(C3CC3)C[C@H]2F)nn2ccc(-c3ccc4nccn4n3)c12] |
| 529 | 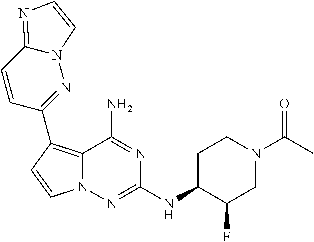 [SMILES: CC(=O)N1CC[C@H](Nc2nc(N)c3c(-c4ccc5nccn5n4)ccn3n2)[C@H](F)C1] |
| 530 | 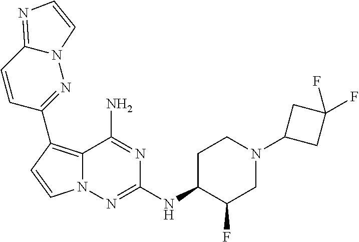 [SMILES: Nc1nc(N[C@H]2CCN(C3CC(F)(F)C3)C[C@H]2F)nn2ccc(-c3ccc4nccn4n3)c12] |
| 531 | 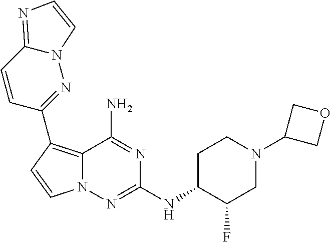 [SMILES: Nc1nc(N[C@@H]2CCN(C3COC3)C[C@@H]2F)nn2ccc(-c3ccc4nccn4n3)c12] |
| 532 | 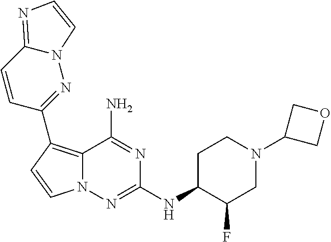 [SMILES: Nc1nc(N[C@H]2CCN(C3COC3)C[C@H]2F)nn2ccc(-c3ccc4nccn4n3)c12] |
| 533 | 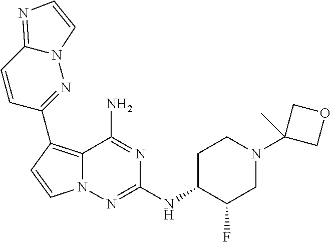 [SMILES: CC1(N2CC[C@@H](Nc3nc(N)c4c(-c5ccc6nccn6n5)ccn4n3)[C@@H](F)C2)COC1] |
| 534 | 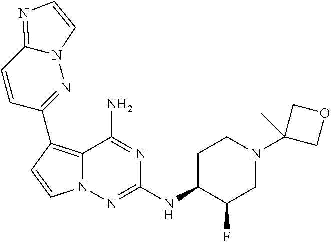 [SMILES: CC1(N2CC[C@H](Nc3nc(N)c4c(-c5ccc6nccn6n5)ccn4n3)[C@H](F)C2)COC1] |
| 535 | 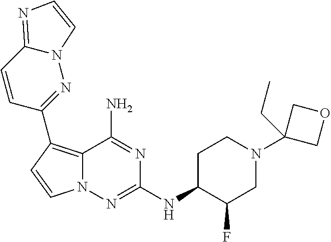 [SMILES: CCC1(N2CC[C@H](Nc3nc(N)c4c(-c5ccc6nccn6n5)ccn4n3)[C@H](F)C2)COC1] |
| 536 | 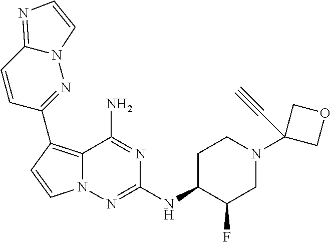 [SMILES: C#CC1(N2CC[C@H](Nc3nc(N)c4c(-c5ccc6nccn6n5)ccn4n3)[C@H](F)C2)COC1] |
| 537 | 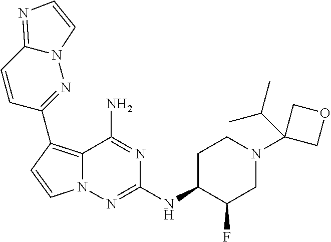 [SMILES: CC(C)C1(N2CC[C@H](Nc3nc(N)c4c(-c5ccc6nccn6n5)ccn4n3)[C@H](F)C2)COC1] |
| 538 | 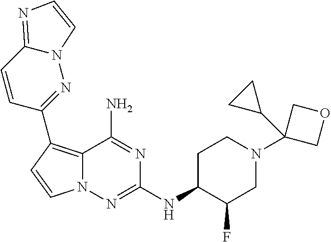 [SMILES: Nc1nc(N[C@H]2CCN(C3(C4CC4)COC3)C[C@H]2F)nn2ccc(-c3ccc4nccn4n3)c12] |
| 539 | 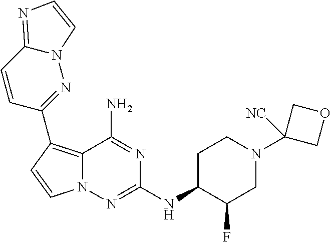 [SMILES: N#CC1(N2CC[C@H](Nc3nc(N)c4c(-c5ccc6nccn6n5)ccn4n3)[C@H](F)C2)COC1] |
| 540 | 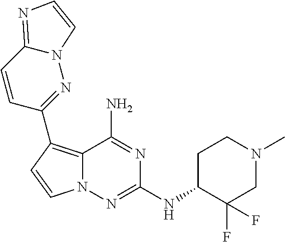 [SMILES: CN1CC[C@@H](Nc2nc(N)c3c(-c4ccc5nccn5n4)ccn3n2)C(F)(F)C1] |
| 541 | 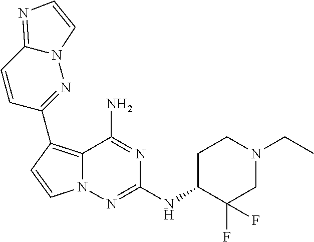 [SMILES: CCN1CC[C@@H](Nc2nc(N)c3c(-c4ccc5nccn5n4)ccn3n2)C(F)(F)C1] |
| 542 | 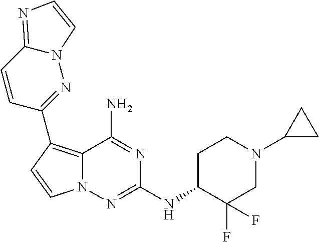 [SMILES: Nc1nc(N[C@@H]2CCN(C3CC3)CC2(F)F)nn2ccc(-c3ccc4nccn4n3)c12] |
| 543 | 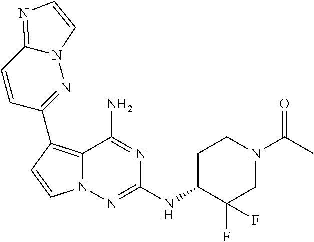 [SMILES: CC(=O)N1CC[C@@H](Nc2nc(N)c3c(-c4ccc5nccn5n4)ccn3n2)C(F)(F)C1] |
| 544 | 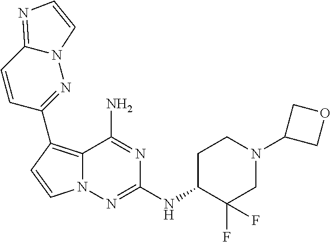 [SMILES: Nc1nc(N[C@@H]2CCN(C3COC3)CC2(F)F)nn2ccc(-c3ccc4nccn4n3)c12] |
| 545 | 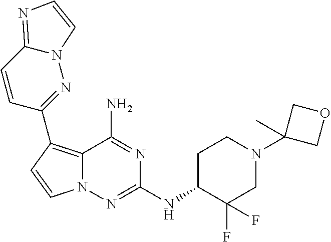 [SMILES: CC1(N2CC[C@@H](Nc3nc(N)c4c(-c5ccc6nccn6n5)ccn4n3)C(F)(F)C2)COC1] |
| 546 | 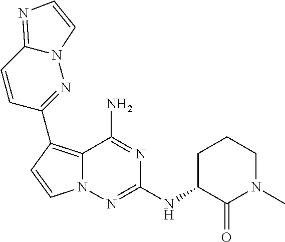 [SMILES: CN1CCC[C@@H](Nc2nc(N)c3c(-c4ccc5nccn5n4)ccn3n2)C1=O] |
| 547 | 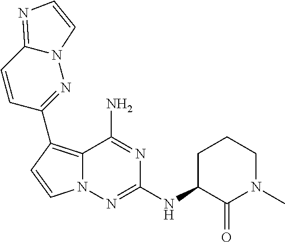 [SMILES: CN1CCC[C@H](Nc2nc(N)c3c(-c4ccc5nccn5n4)ccn3n2)C1=O] |
| 548 | 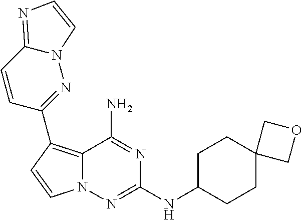 [SMILES: Nc1nc(NC2CCC3(CC2)COC3)nn2ccc(-c3ccc4nccn4n3)c12] |
| 549 | 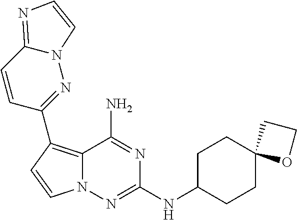 |
| 550 | 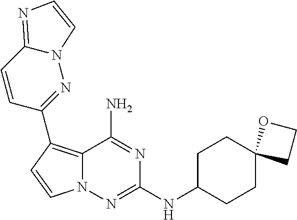 |
| 551 | 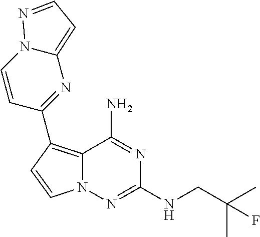 [SMILES: CC(C)(F)CNc1nc(N)c2c(-c3ccn4nccc4n3)ccn2n1] |
| 552 | 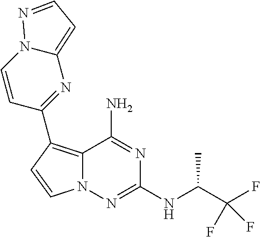 [SMILES: C[C@@H](Nc1nc(N)c2c(-c3ccn4nccc4n3)ccn2n1)C(F)(F)F] |
| 553 | 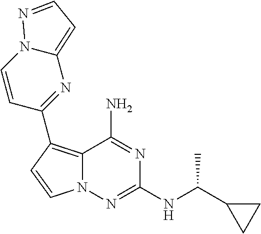 [SMILES: C[C@@H](Nc1nc(N)c2c(-c3ccn4nccc4n3)ccn2n1)C1CC1] |
| 554 | 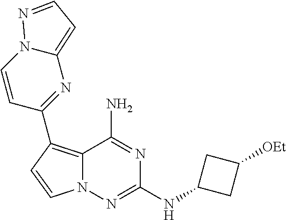 [SMILES: CCO[C@H]1C[C@@H](Nc2nc(N)c3c(-c4ccn5nccc5n4)ccn3n2)C1] |
| 555 | 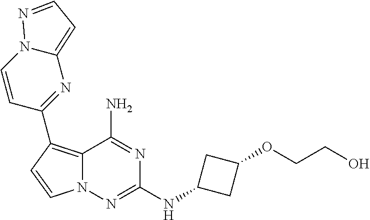 [SMILES: Nc1nc(N[C@H]2C[C@@H](OCCO)C2)nn2ccc(-c3ccn4nccc4n3)c12] |
| 556 | 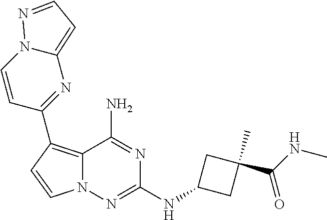 [SMILES: CNC(=O)[C@]1(C)C[C@@H](Nc2nc(N)c3c(-c4ccn5nccc5n4)ccn3n2)C1] |
| 557 | 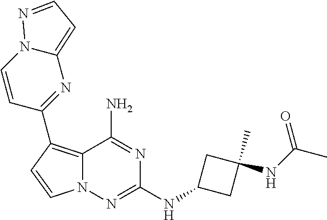 [SMILES: CC(=O)N[C@]1(C)C[C@@H](Nc2nc(N)c3c(-c4ccn5nccc5n4)ccn3n2)C1] |
| 558 | 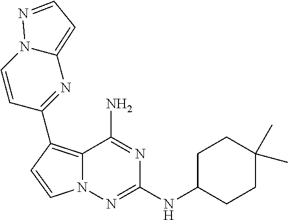 [SMILES: CC1(C)CCC(Nc2nc(N)c3c(-c4ccn5nccc5n4)ccn3n2)CC1] |
| 559 | 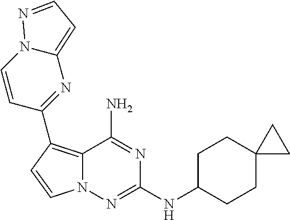 [SMILES: Nc1nc(NC2CCC3(CC2)CC3)nn2ccc(-c3ccn4nccc4n3)c12] |
| 560 | 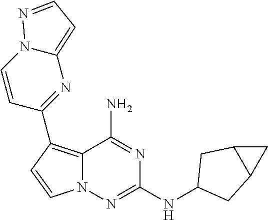 [SMILES: Nc1nc(NC2CC3CC3C2)nn2ccc(-c3ccn4nccc4n3)c12] |
| 561 | 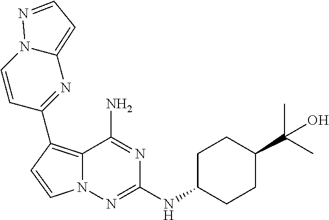 [SMILES: CC(C)(O)[C@H]1CC[C@H](Nc2nc(N)c3c(-c4ccn5nccc5n4)ccn3n2)CC1] |
| 562 | 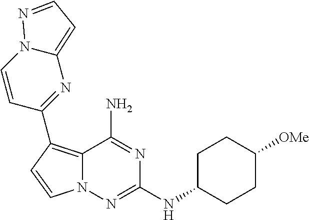 [SMILES: CO[C@H]1CC[C@@H](Nc2nc(N)c3c(-c4ccn5nccc5n4)ccn3n2)CC1] |
| 563 | 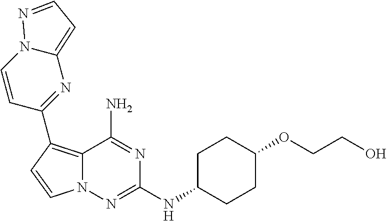 [SMILES: Nc1nc(N[C@H]2CC[C@@H](OCCO)CC2)nn2ccc(-c3ccn4nccc4n3)c12] |
| 564 | 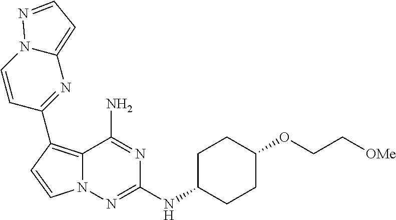 [SMILES: COCCO[C@H]1CC[C@@H](Nc2nc(N)c3c(-c4ccn5nccc5n4)ccn3n2)CC1] |
| 565 | 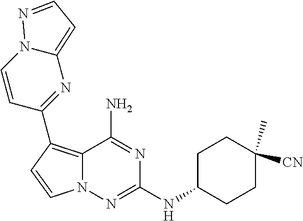 [SMILES: C[C@]1(C#N)CC[C@H](Nc2nc(N)c3c(-c4ccn5nccc5n4)ccn3n2)CC1] |
| 566 | 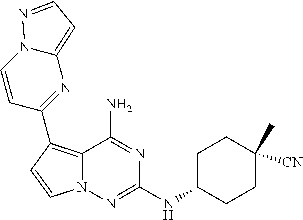 [SMILES: C[C@]1(C#N)CC[C@@H](Nc2nc(N)c3c(-c4ccn5nccc5n4)ccn3n2)CC1] |
| 567 | 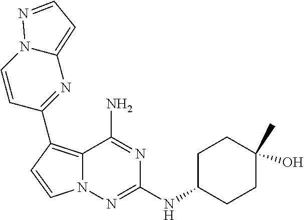 [SMILES: C[C@]1(O)CC[C@@H](Nc2nc(N)c3c(-c4ccn5nccc5n4)ccn3n2)CC1] |
| 568 | 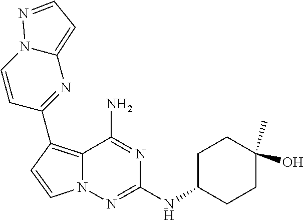 [SMILES: C[C@]1(O)CC[C@H](Nc2nc(N)c3c(-c4ccn5nccc5n4)ccn3n2)CC1] |
| 569 | 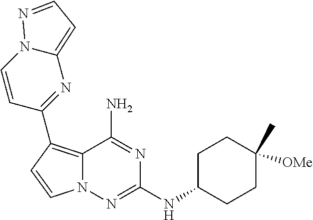 [SMILES: CO[C@]1(C)CC[C@H](Nc2nc(N)c3c(-c4ccn5nccc5n4)ccn3n2)CC1] |
| 570 | 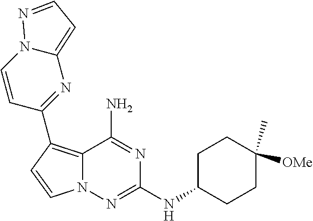 [SMILES: CO[C@]1(C)CC[C@@H](Nc2nc(N)c3c(-c4ccn5nccc5n4)ccn3n2)CC1] |
| 571 | 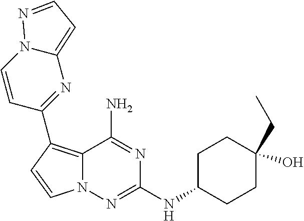 [SMILES: CC[C@]1(O)CC[C@@H](Nc2nc(N)c3c(-c4ccn5nccc5n4)ccn3n2)CC1] |
| 572 | 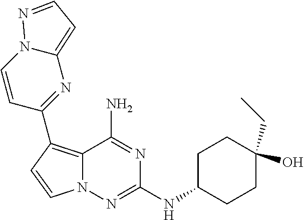 [SMILES: CC[C@]1(O)CC[C@H](Nc2nc(N)c3c(-c4ccn5nccc5n4)ccn3n2)CC1] |
| 573 | 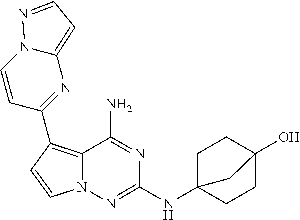 [SMILES: Nc1nc(NC23CCC(O)(CC2)C3)nn2ccc(-c3ccn4nccc4n3)c12] |
| 574 | 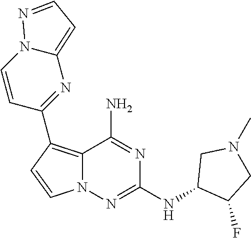 [SMILES: CN1C[C@H](F)[C@H](Nc2nc(N)c3c(-c4ccn5nccc5n4)ccn3n2)C1] |
| 575 | 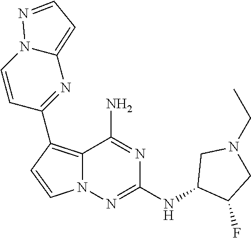 [SMILES: CCN1C[C@H](F)[C@H](Nc2nc(N)c3c(-c4ccn5nccc5n4)ccn3n2)C1] |
| 576 | 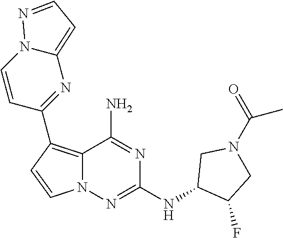 [SMILES: CC(=O)N1C[C@H](F)[C@H](Nc2nc(N)c3c(-c4ccn5nccc5n4)ccn3n2)C1] |
| 577 | 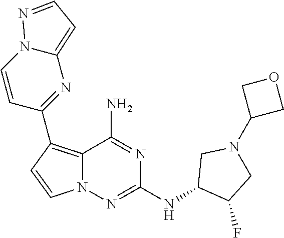 [SMILES: Nc1nc(N[C@@H]2CN(C3COC3)C[C@@H]2F)nn2ccc(-c3ccn4nccc4n3)c12] |
| 578 | 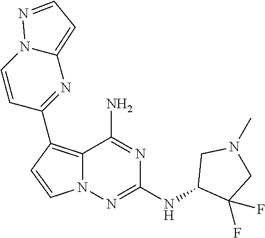 [SMILES: CN1C[C@@H](Nc2nc(N)c3c(-c4ccn5nccc5n4)ccn3n2)C(F)(F)C1] |
| 579 | 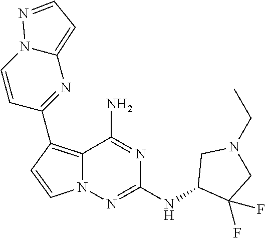 [SMILES: CCN1C[C@@H](Nc2nc(N)c3c(-c4ccn5nccc5n4)ccn3n2)C(F)(F)C1] |
| 580 | 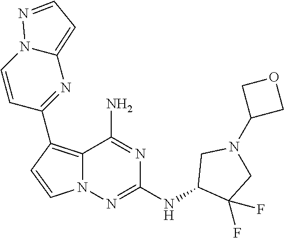 [SMILES: Nc1nc(N[C@@H]2CN(C3COC3)CC2(F)F)nn2ccc(-c3ccn4nccc4n3)c12] |
| 581 | 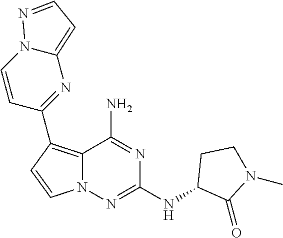 [SMILES: CN1CC[C@@H](Nc2nc(N)c3c(-c4ccn5nccc5n4)ccn3n2)C1=O] |
| 582 | 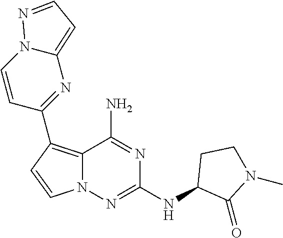 [SMILES: CN1CC[C@H](Nc2nc(N)c3c(-c4ccn5nccc5n4)ccn3n2)C1=O] |
| 583 | 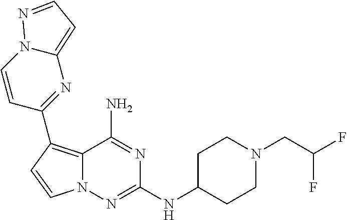 [SMILES: Nc1nc(NC2CCN(CC(F)F)CC2)nn2ccc(-c3ccn4nccc4n3)c12] |
| 584 | 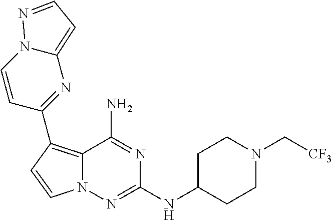 [SMILES: Nc1nc(NC2CCN(CC(F)(F)F)CC2)nn2ccc(-c3ccn4nccc4n3)c12] |
| 585 | 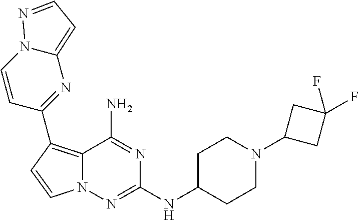 [SMILES: Nc1nc(NC2CCN(C3CC(F)(F)C3)CC2)nn2ccc(-c3ccn4nccc4n3)c12] |
| 586 | 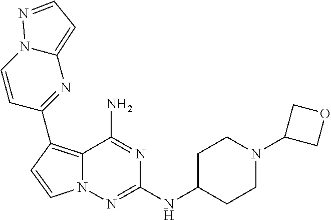 [SMILES: Nc1nc(NC2CCN(C3COC3)CC2)nn2ccc(-c3ccn4nccc4n3)c12] |
| 587 | 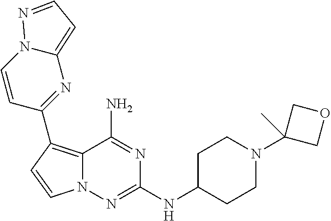 [SMILES: CC1(N2CCC(Nc3nc(N)c4c(-c5ccn6nccc6n5)ccn4n3)CC2)COC1] |
| 588 | 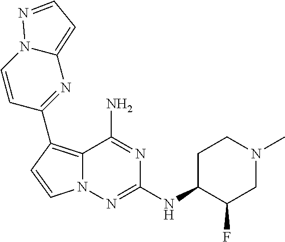 [SMILES: CN1CC[C@H](Nc2nc(N)c3c(-c4ccn5nccc5n4)ccn3n2)[C@H](F)C1] |
| 589 | 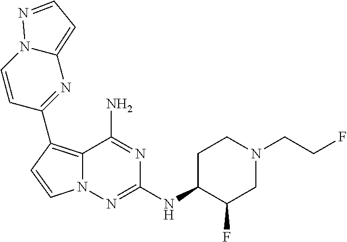 [SMILES: Nc1nc(N[C@H]2CCN(CCF)C[C@H]2F)nn2ccc(-c3ccn4nccc4n3)c12] |
| 590 | 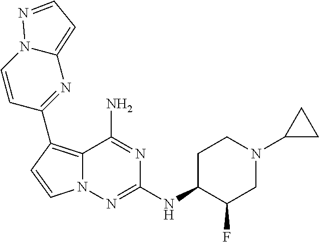 [SMILES: Nc1nc(N[C@H]2CCN(C3CC3)C[C@H]2F)nn2ccc(-c3ccn4nccc4n3)c12] |
| 591 | 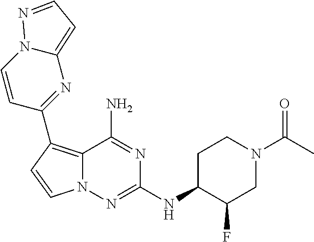 [SMILES: CC(=O)N1CC[C@H](Nc2nc(N)c3c(-c4ccn5nccc5n4)ccn3n2)[C@H](F)C1] |
| 592 | 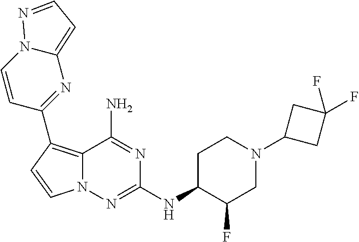 [SMILES: Nc1nc(N[C@H]2CCN(C3CC(F)(F)C3)C[C@H]2F)nn2ccc(-c3ccn4nccc4n3)c12] |
| 593 | 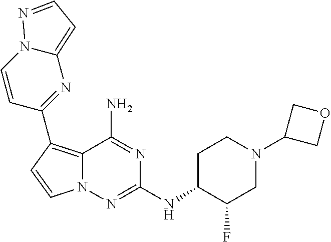 [SMILES: Nc1nc(N[C@@H]2CCN(C3COC3)C[C@@H]2F)nn2ccc(-c3ccn4nccc4n3)c12] |
| 594 | 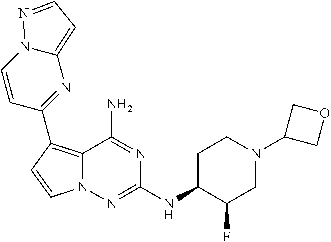 [SMILES: Nc1nc(N[C@H]2CCN(C3COC3)C[C@H]2F)nn2ccc(-c3ccn4nccc4n3)c12] |
| 595 | 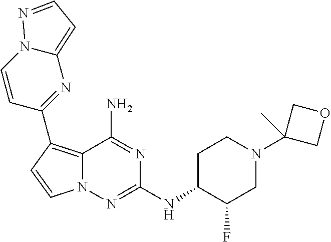 [SMILES: CC1(N2CC[C@@H](Nc3nc(N)c4c(-c5ccn6nccc6n5)ccn4n3)[C@@H](F)C2)COC1] |
| 596 | 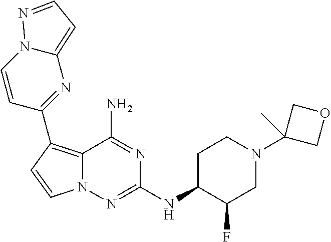 [SMILES: CC1(N2CC[C@H](Nc3nc(N)c4c(-c5ccn6nccc6n5)ccn4n3)[C@H](F)C2)COC1] |
| 597 | 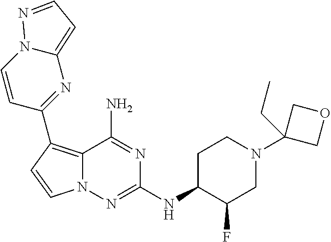 [SMILES: CCC1(N2CC[C@H](Nc3nc(N)c4c(-c5ccn6nccc6n5)ccn4n3)[C@H](F)C2)COC1] |
| 598 | 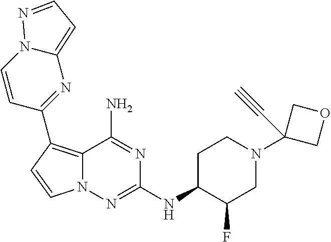 [SMILES: C#CC1(N2CC[C@H](Nc3nc(N)c4c(-c5ccn6nccc6n5)ccn4n3)[C@H](F)C2)COC1] |
| 599 | 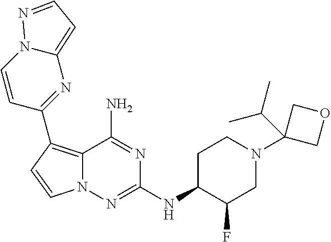 [SMILES: CC(C)C1(N2CC[C@H](Nc3nc(N)c4c(-c5ccn6nccc6n5)ccn4n3)[C@H](F)C2)COC1] |
| 600 | 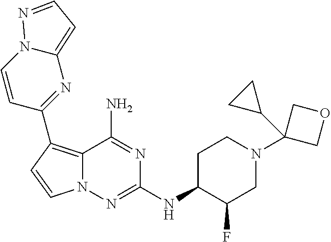 [SMILES: Nc1nc(N[C@H]2CCN(C3(C4CC4)COC3)C[C@H]2F)nn2ccc(-c3ccn4nccc4n3)c12] |
| 601 | 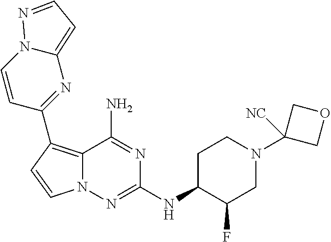 [SMILES: N#CC1(N2CC[C@H](Nc3nc(N)c4c(-c5ccn6nccc6n5)ccn4n3)[C@H](F)C2)COC1] |
| 602 | 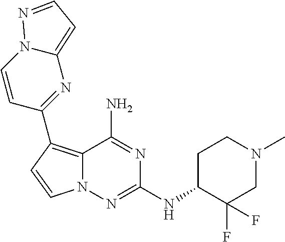 [SMILES: CN1CC[C@@H](Nc2nc(N)c3c(-c4ccn5nccc5n4)ccn3n2)C(F)(F)C1] |
| 603 | 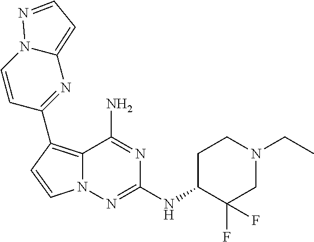 [SMILES: CCN1CC[C@@H](Nc2nc(N)c3c(-c4ccn5nccc5n4)ccn3n2)C(F)(F)C1] |
| 604 | 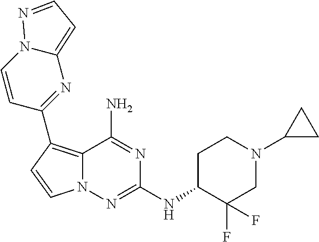 [SMILES: Nc1nc(N[C@@H]2CCN(C3CC3)CC2(F)F)nn2ccc(-c3ccn4nccc4n3)c12] |
| 605 | 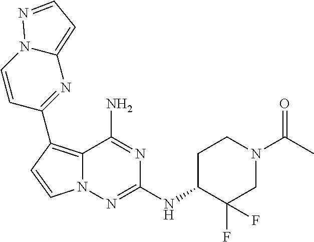 [SMILES: CC(=O)N1CC[C@@H](Nc2nc(N)c3c(-c4ccn5nccc5n4)ccn3n2)C(F)(F)C1] |
| 606 | 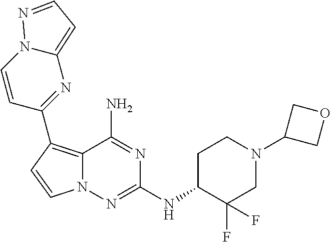 [SMILES: Nc1nc(N[C@@H]2CCN(C3COC3)CC2(F)F)nn2ccc(-c3ccn4nccc4n3)c12] |
| 607 | 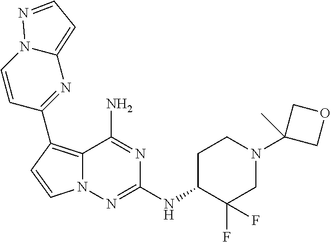 [SMILES: CC1(N2CC[C@@H](Nc3nc(N)c4c(-c5ccn6nccc6n5)ccn4n3)C(F)(F)C2)COC1] |
| 608 | 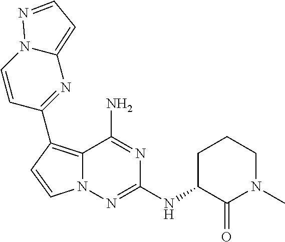 [SMILES: CN1CCC[C@@H](Nc2nc(N)c3c(-c4ccn5nccc5n4)ccn3n2)C1=O] |
| 609 | 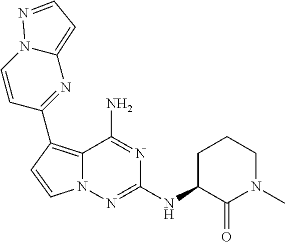 [SMILES: CN1CCC[C@H](Nc2nc(N)c3c(-c4ccn5nccc5n4)ccn3n2)C1=O] |
| 610 | 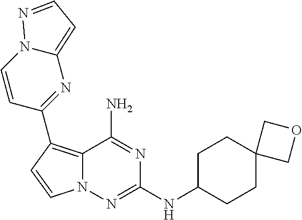 [SMILES: Nc1nc(NC2CCC3(CC2)COC3)nn2ccc(-c3ccn4nccc4n3)c12] |
| 611 | 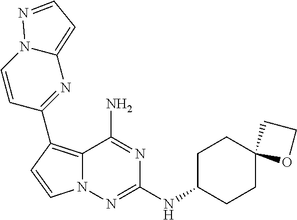 [SMILES: Nc1nc(N[C@H]2CC[C@@]3(CCO3)CC2)nn2ccc(-c3ccn4nccc4n3)c12] |
| 612 | 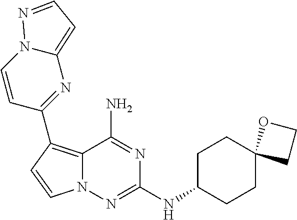 [SMILES: Nc1nc(N[C@H]2CC[C@]3(CCO3)CC2)nn2ccc(-c3ccn4nccc4n3)c12] |
| 613 | 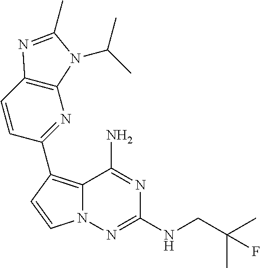 [SMILES: Cc1nc2ccc(-c3ccn4nc(NCC(C)(C)F)nc(N)c34)nc2n1C(C)C] |
| 614 | 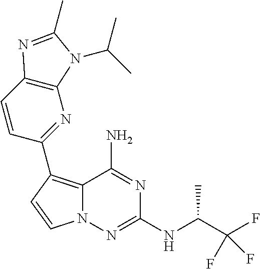 [SMILES: Cc1nc2ccc(-c3ccn4nc(N[C@H](C)C(F)(F)F)nc(N)c34)nc2n1C(C)C] |
| 615 | 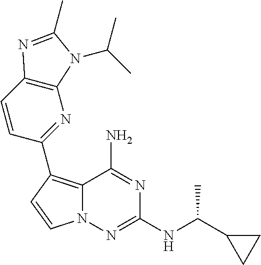 [SMILES: Cc1nc2ccc(-c3ccn4nc(N[C@H](C)C5CC5)nc(N)c34)nc2n1C(C)C] |
| 616 | 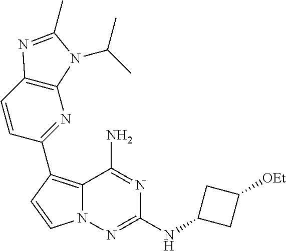 [SMILES: CCO[C@H]1C[C@@H](Nc2nc(N)c3c(-c4ccc5nc(C)n(C(C)C)c5n4)ccn3n2)C1] |
| 617 | 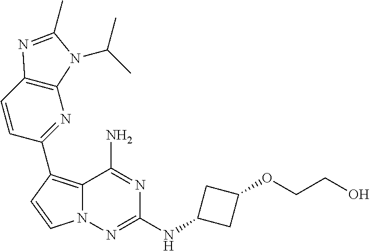 [SMILES: Cc1nc2ccc(-c3ccn4nc(N[C@H]5C[C@@H](OCCO)C5)nc(N)c34)nc2n1C(C)C] |
| 618 | 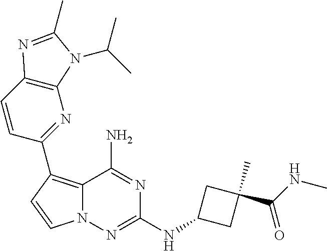 [SMILES: CNC(=O)[C@]1(C)C[C@@H](Nc2nc(N)c3c(-c4ccc5nc(C)n(C(C)C)c5n4)ccn3n2)C1] |
| 619 | 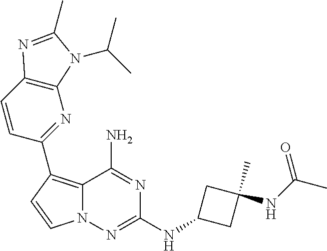 [SMILES: CC(=O)N[C@]1(C)C[C@@H](Nc2nc(N)c3c(-c4ccc5nc(C)n(C(C)C)c5n4)ccn3n2)C1] |
| 620 | 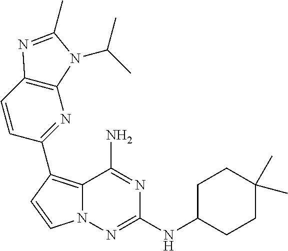 [SMILES: Cc1nc2ccc(-c3ccn4nc(NC5CCC(C)(C)CC5)nc(N)c34)nc2n1C(C)C] |
| 621 | 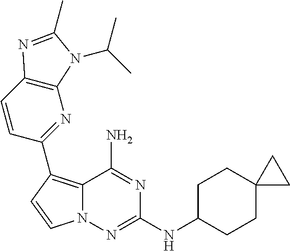 [SMILES: Cc1nc2ccc(-c3ccn4nc(NC5CCC6(CC5)CC6)nc(N)c34)nc2n1C(C)C] |
| 622 | 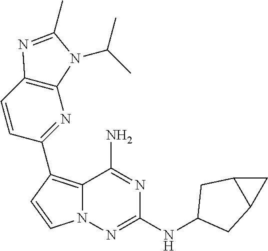 [SMILES: Cc1nc2ccc(-c3ccn4nc(NC5CC6CC6C5)nc(N)c34)nc2n1C(C)C] |
| 623 | 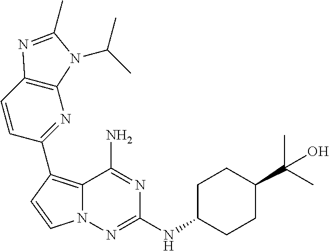 [SMILES: Cc1nc2ccc(-c3ccn4nc(N[C@H]5CC[C@H](C(C)(C)O)CC5)nc(N)c34)nc2n1C(C)C] |
| 624 | 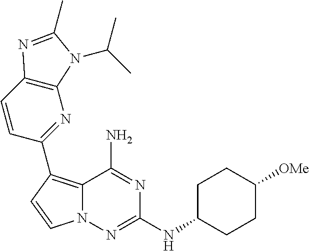 [SMILES: CO[C@H]1CC[C@@H](Nc2nc(N)c3c(-c4ccc5nc(C)n(C(C)C)c5n4)ccn3n2)CC1] |
| 625 | 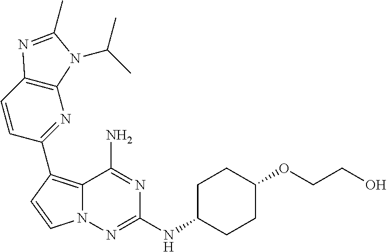 [SMILES: Cc1nc2ccc(-c3ccn4nc(N[C@H]5CC[C@@H](OCCO)CC5)nc(N)c34)nc2n1C(C)C] |
| 626 | 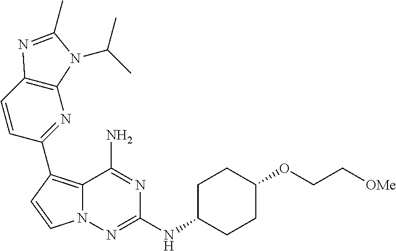 [SMILES: COCCO[C@H]1CC[C@@H](Nc2nc(N)c3c(-c4ccc5nc(C)n(C(C)C)c5n4)ccn3n2)CC1] |
| 627 | 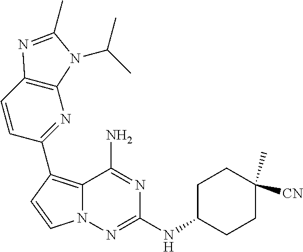 [SMILES: Cc1nc2ccc(-c3ccn4nc(N[C@H]5CC[C@](C)(C#N)CC5)nc(N)c34)nc2n1C(C)C] |
| 628 | 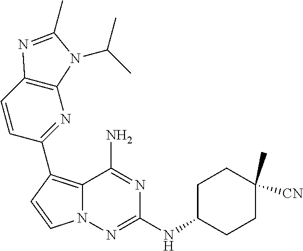 [SMILES: Cc1nc2ccc(-c3ccn4nc(N[C@H]5CC[C@@](C)(C#N)CC5)nc(N)c34)nc2n1C(C)C] |
| 629 | 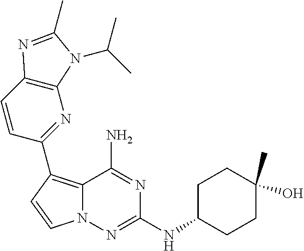 [SMILES: Cc1nc2ccc(-c3ccn4nc(N[C@H]5CC[C@@](C)(O)CC5)nc(N)c34)nc2n1C(C)C] |
| 630 | 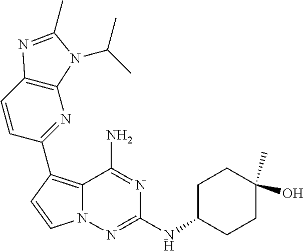 [SMILES: Cc1nc2ccc(-c3ccn4nc(N[C@H]5CC[C@](C)(O)CC5)nc(N)c34)nc2n1C(C)C] |
| 631 | 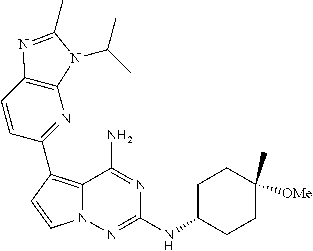 [SMILES: CO[C@]1(C)CC[C@H](Nc2nc(N)c3c(-c4ccc5nc(C)n(C(C)C)c5n4)ccn3n2)CC1] |
| 632 | 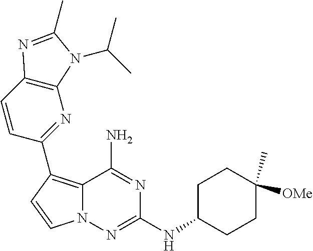 [SMILES: CO[C@]1(C)CC[C@@H](Nc2nc(N)c3c(-c4ccc5nc(C)n(C(C)C)c5n4)ccn3n2)CC1] |
| 633 | 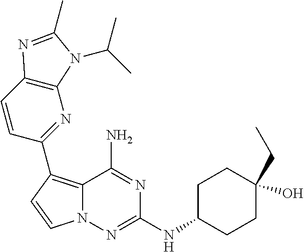 [SMILES: CC[C@]1(O)CC[C@@H](Nc2nc(N)c3c(-c4ccc5nc(C)n(C(C)C)c5n4)ccn3n2)CC1] |
| 634 | 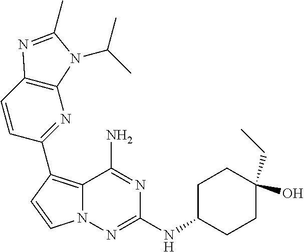 [SMILES: CC[C@]1(O)CC[C@H](Nc2nc(N)c3c(-c4ccc5nc(C)n(C(C)C)c5n4)ccn3n2)CC1] |
| 635 | 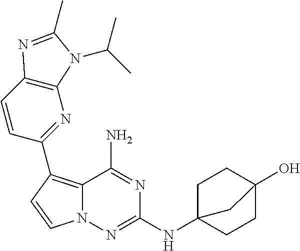 [SMILES: Cc1nc2ccc(-c3ccn4nc(NC56CCC(O)(CC5)C6)nc(N)c34)nc2n1C(C)C] |
| 636 | 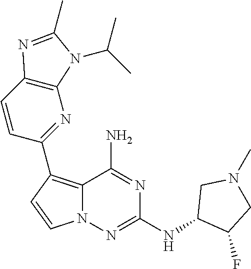 [SMILES: Cc1nc2ccc(-c3ccn4nc(N[C@@H]5CN(C)C[C@@H]5F)nc(N)c34)nc2n1C(C)C] |
| 637 | 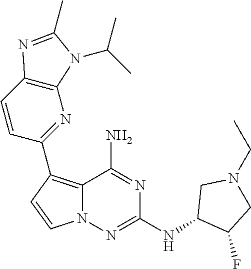 [SMILES: CCN1C[C@H](F)[C@H](Nc2nc(N)c3c(-c4ccc5nc(C)n(C(C)C)c5n4)ccn3n2)C1] |
| 638 | 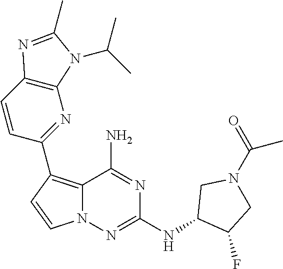 [SMILES: CC(=O)N1C[C@H](F)[C@H](Nc2nc(N)c3c(-c4ccc5nc(C)n(C(C)C)c5n4)ccn3n2)C1] |
| 639 | 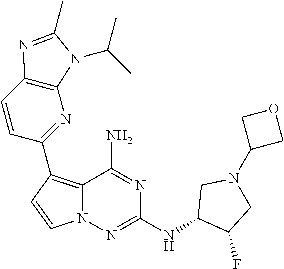 [SMILES: Cc1nc2ccc(-c3ccn4nc(N[C@@H]5CN(C6COC6)C[C@@H]5F)nc(N)c34)nc2n1C(C)C] |
| 640 | 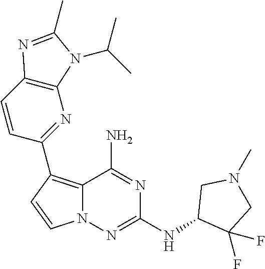 [SMILES: Cc1nc2ccc(-c3ccn4nc(N[C@@H]5CN(C)CC5(F)F)nc(N)c34)nc2n1C(C)C] |
| 641 | 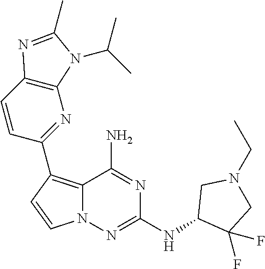 [SMILES: CCN1C[C@@H](Nc2nc(N)c3c(-c4ccc5nc(C)n(C(C)C)c5n4)ccn3n2)C(F)(F)C1] |
| 642 | 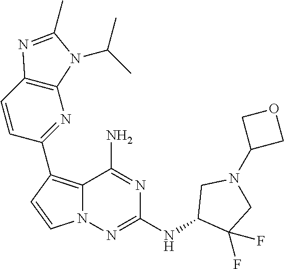 [SMILES: Cc1nc2ccc(-c3ccn4nc(N[C@@H]5CN(C6COC6)CC5(F)F)nc(N)c34)nc2n1C(C)C] |
| 643 | 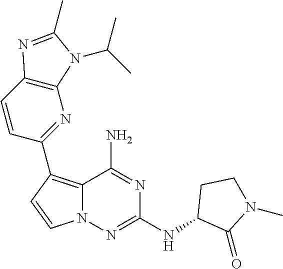 [SMILES: Cc1nc2ccc(-c3ccn4nc(N[C@@H]5CCN(C)C5=O)nc(N)c34)nc2n1C(C)C] |
| 644 | 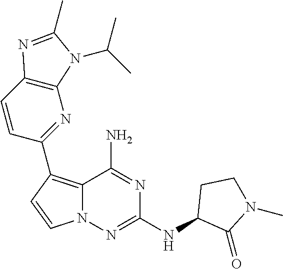 [SMILES: Cc1nc2ccc(-c3ccn4nc(N[C@H]5CCN(C)C5=O)nc(N)c34)nc2n1C(C)C] |
| 645 | 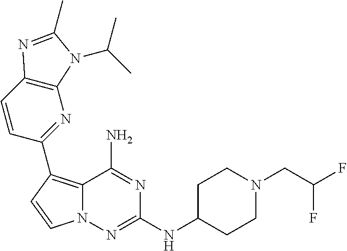 [SMILES: Cc1nc2ccc(-c3ccn4nc(NC5CCN(CC(F)F)CC5)nc(N)c34)nc2n1C(C)C] |
| 646 | 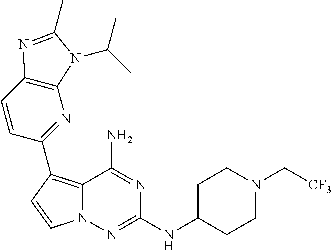 [SMILES: Cc1nc2ccc(-c3ccn4nc(NC5CCN(CC(F)(F)F)CC5)nc(N)c34)nc2n1C(C)C] |
| 647 | 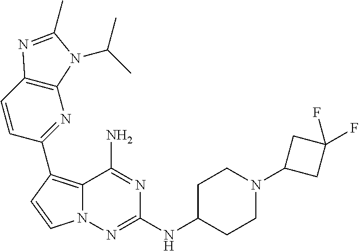 [SMILES: Cc1nc2ccc(-c3ccn4nc(NC5CCN(C6CC(F)(F)C6)CC5)nc(N)c34)nc2n1C(C)C] |
| 648 | 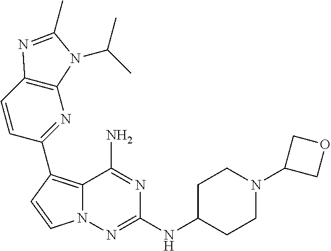 [SMILES: Cc1nc2ccc(-c3ccn4nc(NC5CCN(C6COC6)CC5)nc(N)c34)nc2n1C(C)C] |
| 649 | 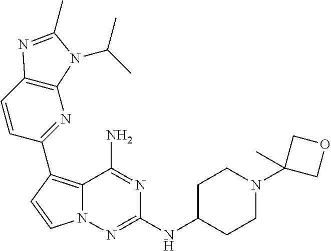 [SMILES: Cc1nc2ccc(-c3ccn4nc(NC5CCN(C6(C)COC6)CC5)nc(N)c34)nc2n1C(C)C] |
| 650 | 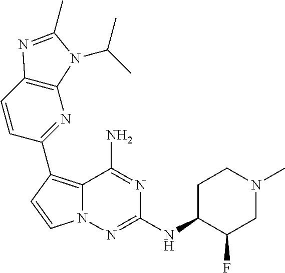 [SMILES: Cc1nc2ccc(-c3ccn4nc(N[C@H]5CCN(C)C[C@H]5F)nc(N)c34)nc2n1C(C)C] |
| 651 | 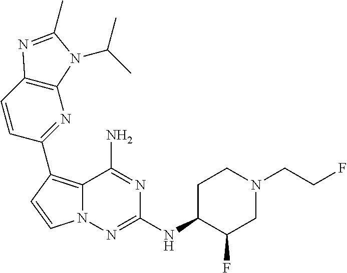 [SMILES: Cc1nc2ccc(-c3ccn4nc(N[C@H]5CCN(CCF)C[C@H]5F)nc(N)c34)nc2n1C(C)C] |
| 652 | 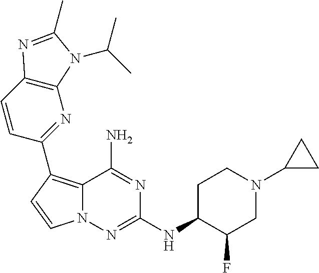 [SMILES: Cc1nc2ccc(-c3ccn4nc(N[C@H]5CCN(C6CC6)C[C@H]5F)nc(N)c34)nc2n1C(C)C] |
| 653 | 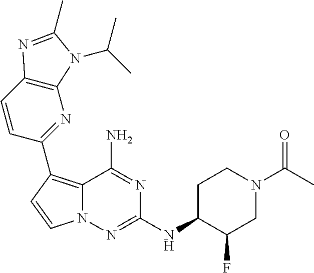 [SMILES: CC(=O)N1CC[C@H](Nc2nc(N)c3c(-c4ccc5nc(C)n(C(C)C)c5n4)ccn3n2)[C@H](F)C1] |
| 654 | 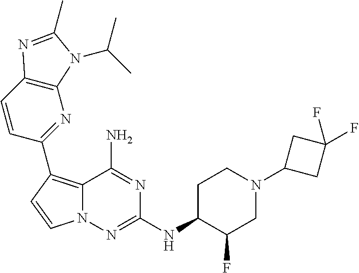 [SMILES: Cc1nc2ccc(-c3ccn4nc(N[C@H]5CCN(C6CC(F)(F)C6)C[C@H]5F)nc(N)c34)nc2n1C(C)C] |
| 655 | 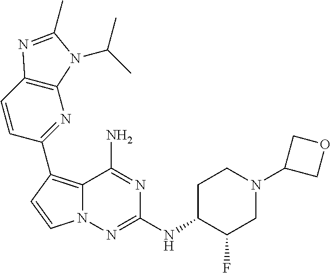 [SMILES: Cc1nc2ccc(-c3ccn4nc(N[C@@H]5CCN(C6COC6)C[C@@H]5F)nc(N)c34)nc2n1C(C)C] |
| 656 | 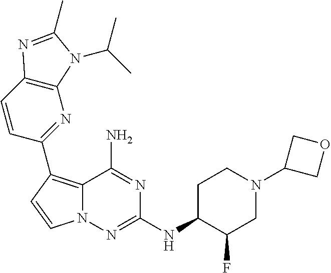 [SMILES: Cc1nc2ccc(-c3ccn4nc(N[C@H]5CCN(C6COC6)C[C@H]5F)nc(N)c34)nc2n1C(C)C] |
| 657 | 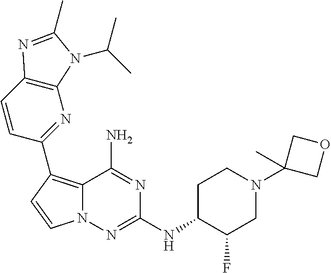 [SMILES: Cc1nc2ccc(-c3ccn4nc(N[C@@H]5CCN(C6(C)COC6)C[C@@H]5F)nc(N)c34)nc2n1C(C)C] |
| 658 | 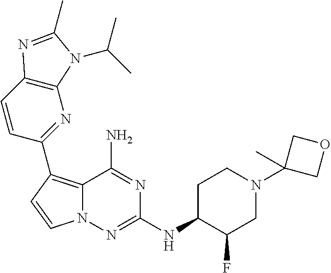 [SMILES: Cc1nc2ccc(-c3ccn4nc(N[C@H]5CCN(C6(C)COC6)C[C@H]5F)nc(N)c34)nc2n1C(C)C] |
| 659 | 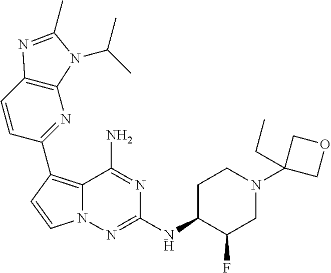 [SMILES: CCC1(N2CC[C@H](Nc3nc(N)c4c(-c5ccc6nc(C)n(C(C)C)c6n5)ccn4n3)[C@H](F)C2)COC1] |
| 660 | 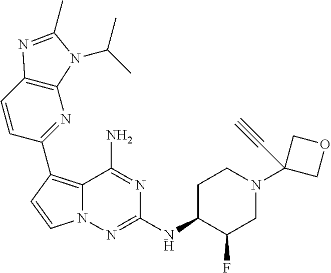 [SMILES: C#CC1(N2CC[C@H](Nc3nc(N)c4c(-c5ccc6nc(C)n(C(C)C)c6n5)ccn4n3)[C@H](F)C2)COC1] |
| 661 | 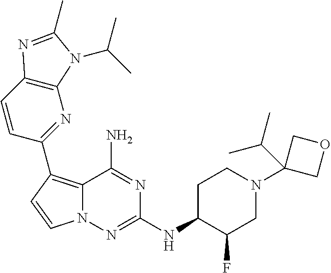 [SMILES: Cc1nc2ccc(-c3ccn4nc(N[C@H]5CCN(C6(C(C)C)COC6)C[C@H]5F)nc(N)c34)nc2n1C(C)C] |
| 662 | 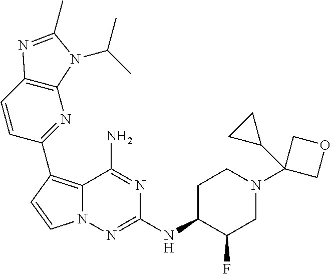 [SMILES: Cc1nc2ccc(-c3ccn4nc(N[C@H]5CCN(C6(C7CC7)COC6)C[C@H]5F)nc(N)c34)nc2n1C(C)C] |
| 663 | 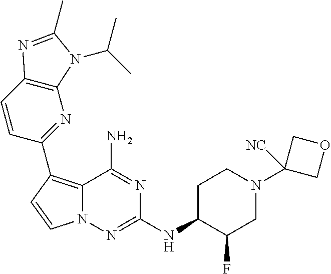 [SMILES: Cc1nc2ccc(-c3ccn4nc(N[C@H]5CCN(C6(C#N)COC6)C[C@H]5F)nc(N)c34)nc2n1C(C)C] |
| 664 | 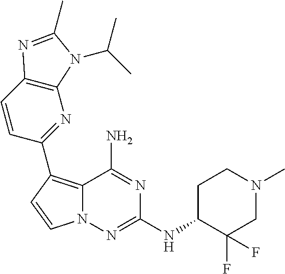 [SMILES: Cc1nc2ccc(-c3ccn4nc(N[C@@H]5CCN(C)CC5(F)F)nc(N)c34)nc2n1C(C)C] |
| 665 | 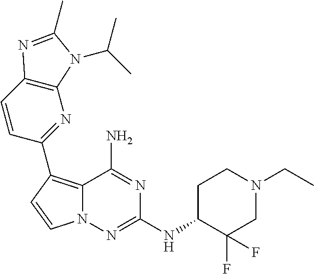 [SMILES: CCN1CC[C@@H](Nc2nc(N)c3c(-c4ccc5nc(C)n(C(C)C)c5n4)ccn3n2)C(F)(F)C1] |
| 666 | 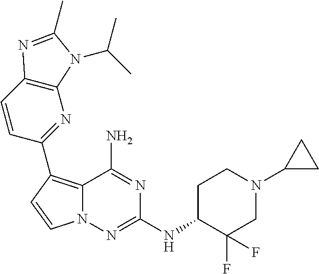 [SMILES: Cc1nc2ccc(-c3ccn4nc(N[C@@H]5CCN(C6CC6)CC5(F)F)nc(N)c34)nc2n1C(C)C] |
| 667 | 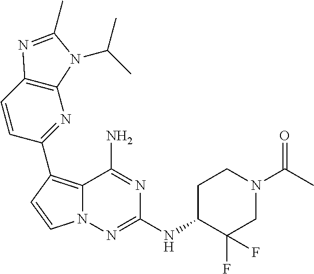 [SMILES: CC(=O)N1CC[C@@H](Nc2nc(N)c3c(-c4ccc5nc(C)n(C(C)C)c5n4)ccn3n2)C(F)(F)C1] |
| 668 | 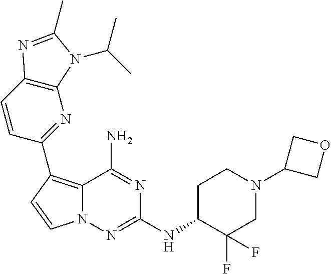 [SMILES: Cc1nc2ccc(-c3ccn4nc(N[C@@H]5CCN(C6COC6)CC5(F)F)nc(N)c34)nc2n1C(C)C] |
| 669 | 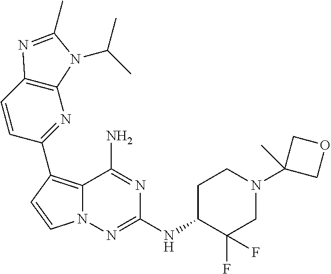 [SMILES: Cc1nc2ccc(-c3ccn4nc(N[C@@H]5CCN(C6(C)COC6)CC5(F)F)nc(N)c34)nc2n1C(C)C] |
| 670 | 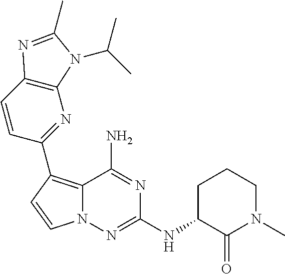 [SMILES: Cc1nc2ccc(-c3ccn4nc(N[C@@H]5CCCN(C)C5=O)nc(N)c34)nc2n1C(C)C] |
| 671 | 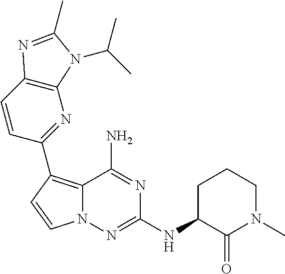 [SMILES: Cc1nc2ccc(-c3ccn4nc(N[C@H]5CCCN(C)C5=O)nc(N)c34)nc2n1C(C)C] |
| 672 | 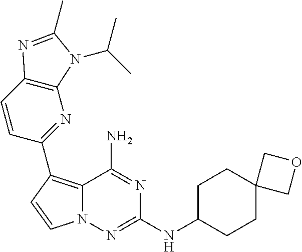 [SMILES: Cc1nc2ccc(-c3ccn4nc(NC5CCC6(CC5)COC6)nc(N)c34)nc2n1C(C)C] |
| 673 | 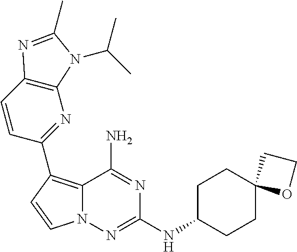 [SMILES: Cc1nc2ccc(-c3ccn4nc(N[C@H]5CC[C@@]6(CCO6)CC5)nc(N)c34)nc2n1C(C)C] |
| 674 | 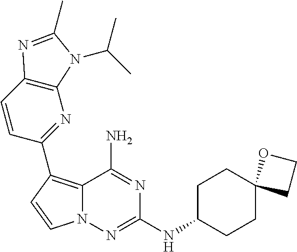 [SMILES: Cc1nc2ccc(-c3ccn4nc(N[C@H]5CC[C@]6(CCO6)CC5)nc(N)c34)nc2n1C(C)C] |
| 675 | 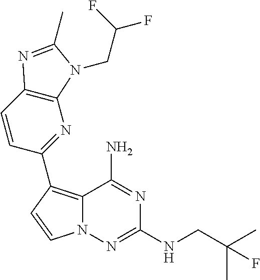 [SMILES: Cc1nc2ccc(-c3ccn4nc(NCC(C)(C)F)nc(N)c34)nc2n1CC(F)F] |
| 676 | 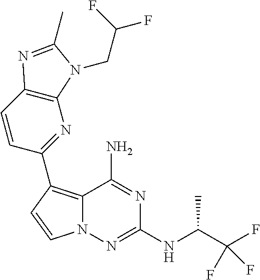 [SMILES: Cc1nc2ccc(-c3ccn4nc(N[C@H](C)C(F)(F)F)nc(N)c34)nc2n1CC(F)F] |
| 677 | 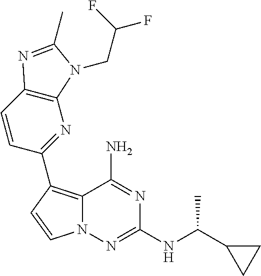 [SMILES: Cc1nc2ccc(-c3ccn4nc(N[C@H](C)C5CC5)nc(N)c34)nc2n1CC(F)F] |
| 678 | 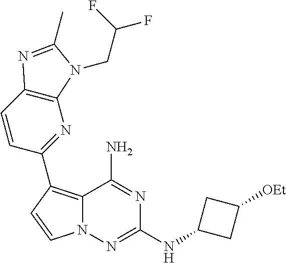 [SMILES: CCO[C@H]1C[C@@H](Nc2nc(N)c3c(-c4ccc5nc(C)n(CC(F)F)c5n4)ccn3n2)C1] |
| 679 | 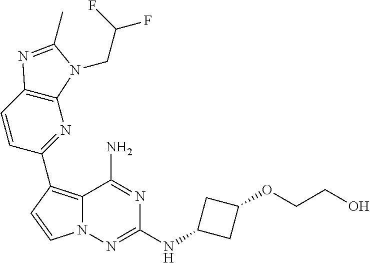 [SMILES: Cc1nc2ccc(-c3ccn4nc(N[C@H]5C[C@@H](OCCO)C5)nc(N)c34)nc2n1CC(F)F] |
| 680 | 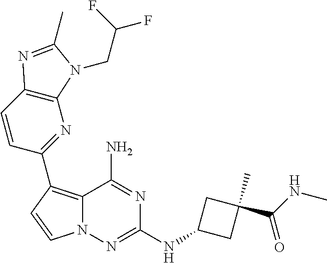 [SMILES: CNC(=O)[C@]1(C)C[C@@H](Nc2nc(N)c3c(-c4ccc5nc(C)n(CC(F)F)c5n4)ccn3n2)C1] |
| 681 | 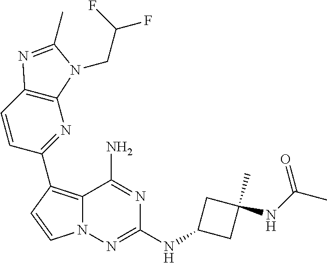 [SMILES: CC(=O)N[C@]1(C)C[C@@H](Nc2nc(N)c3c(-c4ccc5nc(C)n(CC(F)F)c5n4)ccn3n2)C1] |
| 682 | 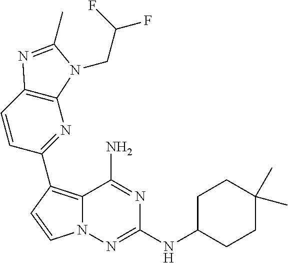 [SMILES: Cc1nc2ccc(-c3ccn4nc(NC5CCC(C)(C)CC5)nc(N)c34)nc2n1CC(F)F] |
| 683 | 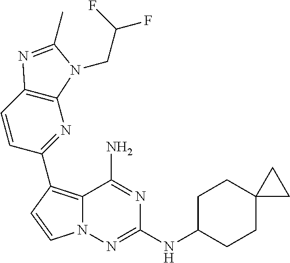 [SMILES: Cc1nc2ccc(-c3ccn4nc(NC5CCC6(CC5)CC6)nc(N)c34)nc2n1CC(F)F] |
| 684 | 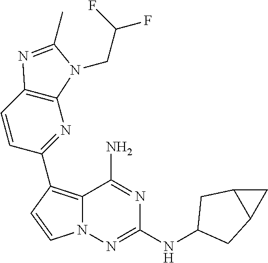 [SMILES: Cc1nc2ccc(-c3ccn4nc(NC5CC6CC6C5)nc(N)c34)nc2n1CC(F)F] |
| 685 | 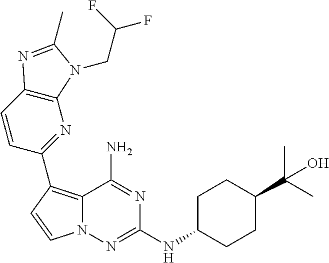 [SMILES: Cc1nc2ccc(-c3ccn4nc(N[C@H]5CC[C@H](C(C)(C)O)CC5)nc(N)c34)nc2n1CC(F)F] |
| 686 | 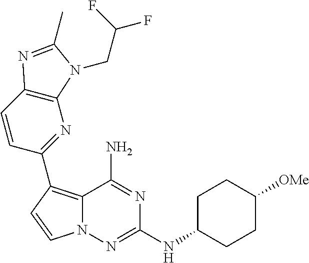 [SMILES: CO[C@H]1CC[C@@H](Nc2nc(N)c3c(-c4ccc5nc(C)n(CC(F)F)c5n4)ccn3n2)CC1] |
| 687 | 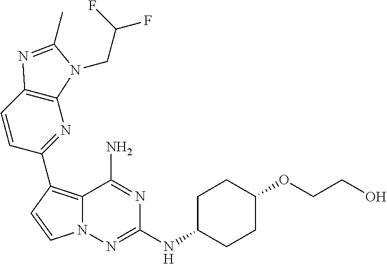 [SMILES: Cc1nc2ccc(-c3ccn4nc(N[C@H]5CC[C@@H](OCCO)CC5)nc(N)c34)nc2n1CC(F)F] |
| 688 | 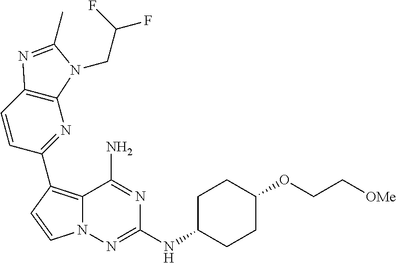 [SMILES: COCCO[C@H]1CC[C@@H](Nc2nc(N)c3c(-c4ccc5nc(C)n(CC(F)F)c5n4)ccn3n2)CC1] |
| 689 | 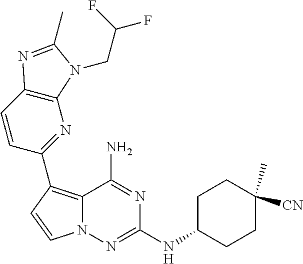 [SMILES: Cc1nc2ccc(-c3ccn4nc(N[C@H]5CC[C@](C)(C#N)CC5)nc(N)c34)nc2n1CC(F)F] |
| 690 | 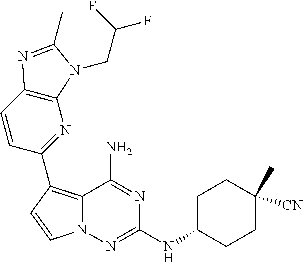 [SMILES: Cc1nc2ccc(-c3ccn4nc(N[C@H]5CC[C@@](C)(C#N)CC5)nc(N)c34)nc2n1CC(F)F] |
| 691 | 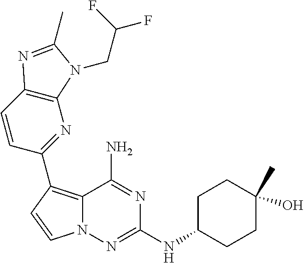 [SMILES: Cc1nc2ccc(-c3ccn4nc(N[C@H]5CC[C@@](C)(O)CC5)nc(N)c34)nc2n1CC(F)F] |
| 692 | 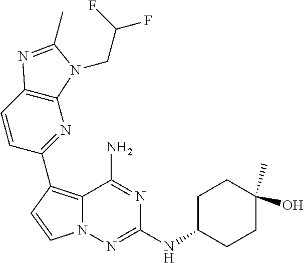 [SMILES: Cc1nc2ccc(-c3ccn4nc(N[C@H]5CC[C@](C)(O)CC5)nc(N)c34)nc2n1CC(F)F] |
| 693 | 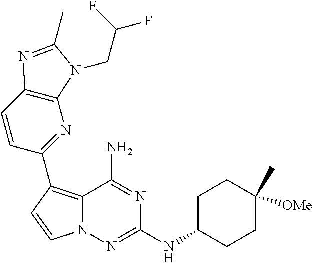 [SMILES: CO[C@]1(C)CC[C@H](Nc2nc(N)c3c(-c4ccc5nc(C)n(CC(F)F)c5n4)ccn3n2)CC1] |
| 694 | 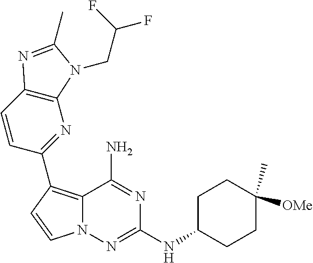 [SMILES: CO[C@]1(C)CC[C@@H](Nc2nc(N)c3c(-c4ccc5nc(C)n(CC(F)F)c5n4)ccn3n2)CC1] |
| 695 | 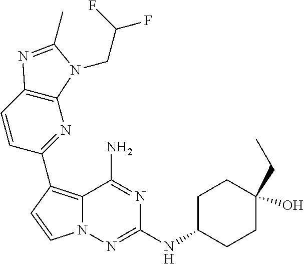 [SMILES: CC[C@]1(O)CC[C@@H](Nc2nc(N)c3c(-c4ccc5nc(C)n(CC(F)F)c5n4)ccn3n2)CC1] |
| 696 | 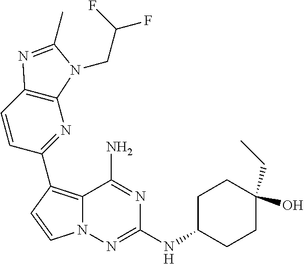 [SMILES: CC[C@]1(O)CC[C@H](Nc2nc(N)c3c(-c4ccc5nc(C)n(CC(F)F)c5n4)ccn3n2)CC1] |
| 697 | 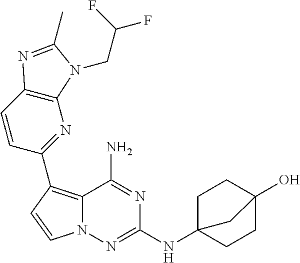 [SMILES: Cc1nc2ccc(-c3ccn4nc(NC56CCC(O)(CC5)C6)nc(N)c34)nc2n1CC(F)F] |
| 698 | 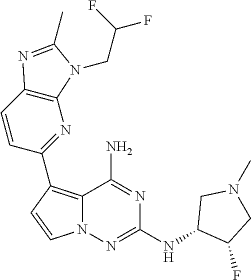 [SMILES: Cc1nc2ccc(-c3ccn4nc(N[C@@H]5CN(C)C[C@@H]5F)nc(N)c34)nc2n1CC(F)F] |
| 699 | 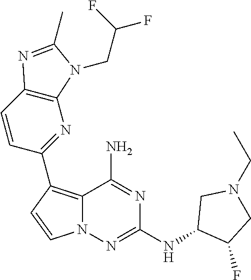 [SMILES: CCN1C[C@H](F)[C@H](Nc2nc(N)c3c(-c4ccc5nc(C)n(CC(F)F)c5n4)ccn3n2)C1] |
| 700 | 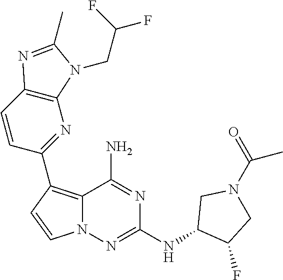 [SMILES: CC(=O)N1C[C@H](F)[C@H](Nc2nc(N)c3c(-c4ccc5nc(C)n(CC(F)F)c5n4)ccn3n2)C1] |
| 701 | 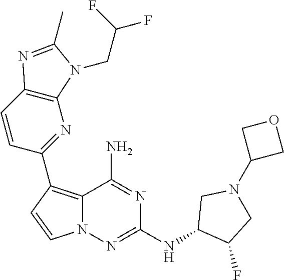 [SMILES: Cc1nc2ccc(-c3ccn4nc(N[C@@H]5CN(C6COC6)C[C@@H]5F)nc(N)c34)nc2n1CC(F)F] |
| 702 | 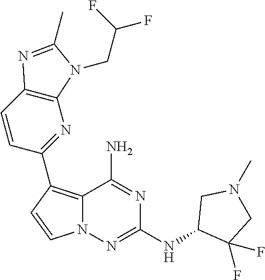 [SMILES: Cc1nc2ccc(-c3ccn4nc(N[C@@H]5CN(C)CC5(F)F)nc(N)c34)nc2n1CC(F)F] |
| 703 | 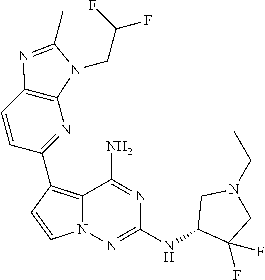 [SMILES: CCN1C[C@@H](Nc2nc(N)c3c(-c4ccc5nc(C)n(CC(F)F)c5n4)ccn3n2)C(F)(F)C1] |
| 704 | 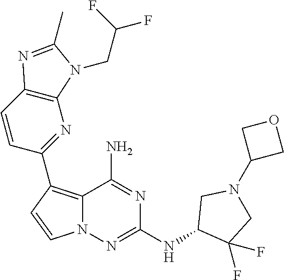 [SMILES: Cc1nc2ccc(-c3ccn4nc(N[C@@H]5CN(C6COC6)CC5(F)F)nc(N)c34)nc2n1CC(F)F] |
| 705 | 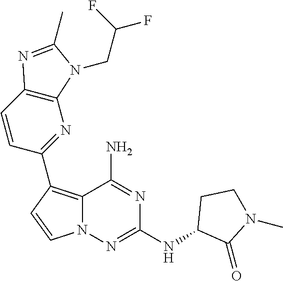 [SMILES: Cc1nc2ccc(-c3ccn4nc(N[C@@H]5CCN(C)C5=O)nc(N)c34)nc2n1CC(F)F] |
| 706 | 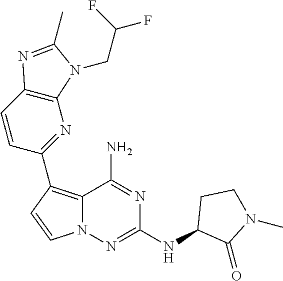 [SMILES: Cc1nc2ccc(-c3ccn4nc(N[C@H]5CCN(C)C5=O)nc(N)c34)nc2n1CC(F)F] |
| 707 | 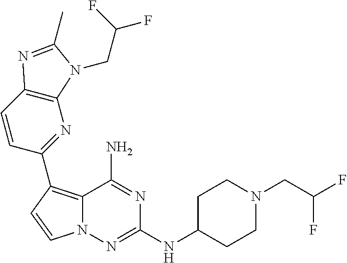 [SMILES: Cc1nc2ccc(-c3ccn4nc(NC5CCN(CC(F)F)CC5)nc(N)c34)nc2n1CC(F)F] |
| 708 | 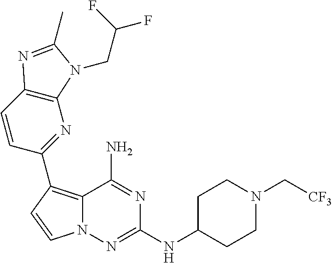 [SMILES: Cc1nc2ccc(-c3ccn4nc(NC5CCN(CC(F)(F)F)CC5)nc(N)c34)nc2n1CC(F)F] |
| 709 | 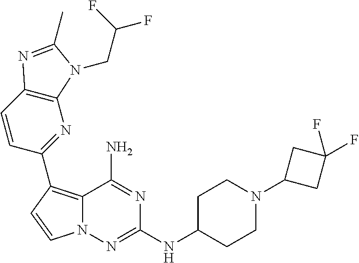 [SMILES: Cc1nc2ccc(-c3ccn4nc(NC5CCN(C6CC(F)(F)C6)CC5)nc(N)c34)nc2n1CC(F)F] |
| 710 | 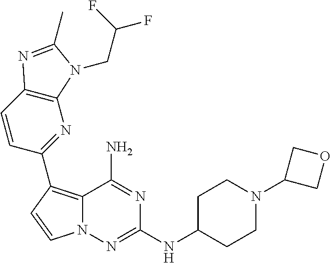 [SMILES: Cc1nc2ccc(-c3ccn4nc(NC5CCN(C6COC6)CC5)nc(N)c34)nc2n1CC(F)F] |
| 711 | 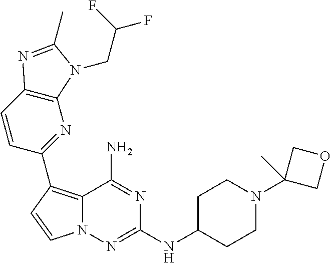 [SMILES: Cc1nc2ccc(-c3ccn4nc(NC5CCN(C6(C)COC6)CC5)nc(N)c34)nc2n1CC(F)F] |
| 712 | 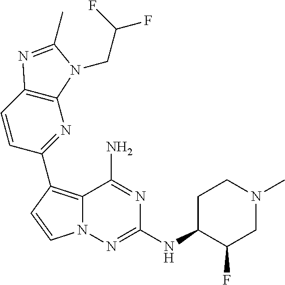 [SMILES: Cc1nc2ccc(-c3ccn4nc(N[C@H]5CCN(C)C[C@H]5F)nc(N)c34)nc2n1CC(F)F] |
| 713 | 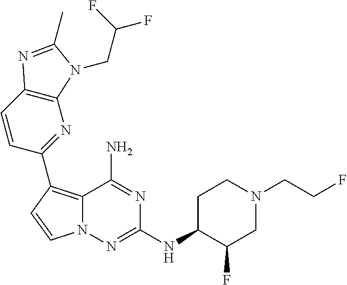 [SMILES: Cc1nc2ccc(-c3ccn4nc(N[C@H]5CCN(CCF)C[C@H]5F)nc(N)c34)nc2n1CC(F)F] |
| 714 | 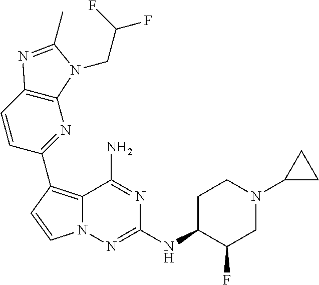 [SMILES: Cc1nc2ccc(-c3ccn4nc(N[C@H]5CCN(C6CC6)C[C@H]5F)nc(N)c34)nc2n1CC(F)F] |
| 715 | 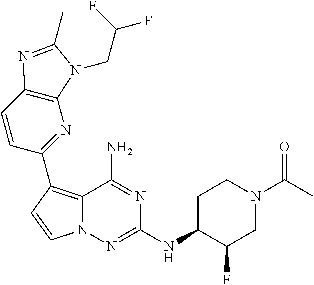 [SMILES: CC(=O)N1CC[C@H](Nc2nc(N)c3c(-c4ccc5nc(C)n(CC(F)F)c5n4)ccn3n2)[C@H](F)C1] |
| 716 | 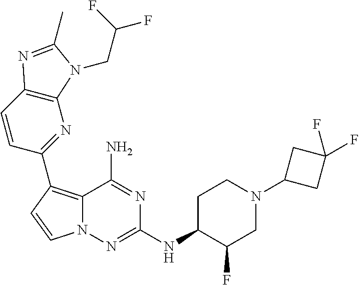 [SMILES: Cc1nc2ccc(-c3ccn4nc(N[C@H]5CCN(C6CC(F)(F)C6)C[C@H]5F)nc(N)c34)nc2n1CC(F)F] |
| 717 | 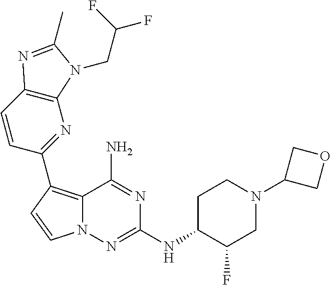 [SMILES: Cc1nc2ccc(-c3ccn4nc(N[C@@H]5CCN(C6COC6)C[C@@H]5F)nc(N)c34)nc2n1CC(F)F] |
| 718 | 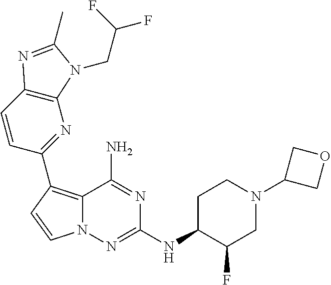 [SMILES: Cc1nc2ccc(-c3ccn4nc(N[C@H]5CCN(C6COC6)C[C@H]5F)nc(N)c34)nc2n1CC(F)F] |
| 719 | 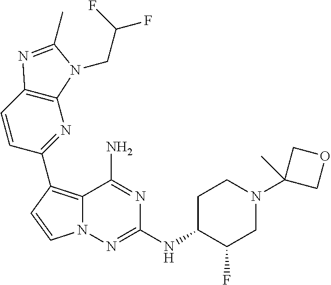 [SMILES: Cc1nc2ccc(-c3ccn4nc(N[C@@H]5CCN(C6(C)COC6)C[C@@H]5F)nc(N)c34)nc2n1CC(F)F] |
| 720 | 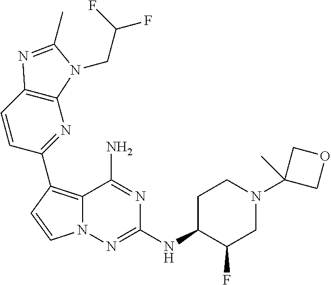 [SMILES: Cc1nc2ccc(-c3ccn4nc(N[C@H]5CCN(C6(C)COC6)C[C@H]5F)nc(N)c34)nc2n1CC(F)F] |
| 721 | 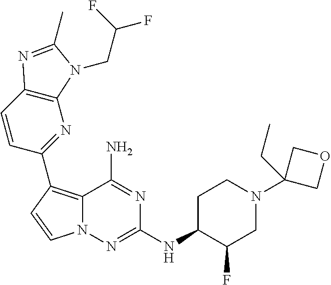 [SMILES: CCC1(N2CC[C@H](Nc3nc(N)c4c(-c5ccc6nc(C)n(CC(F)F)c6n5)ccn4n3)[C@H](F)C2)COC1] |
| 722 | 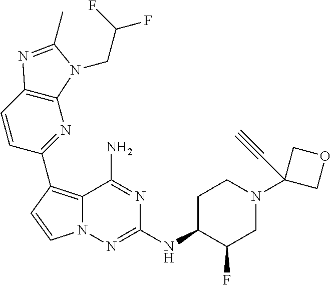 [SMILES: C#CC1(N2CC[C@H](Nc3nc(N)c4c(-c5ccc6nc(C)n(CC(F)F)c6n5)ccn4n3)[C@H](F)C2)COC1] |
| 723 | 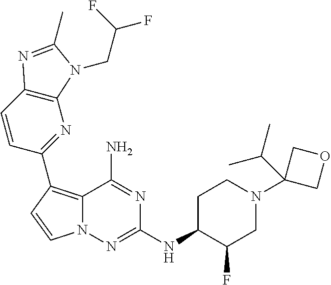 [SMILES: Cc1nc2ccc(-c3ccn4nc(N[C@H]5CCN(C6(C(C)C)COC6)C[C@H]5F)nc(N)c34)nc2n1CC(F)F] |
| 724 | 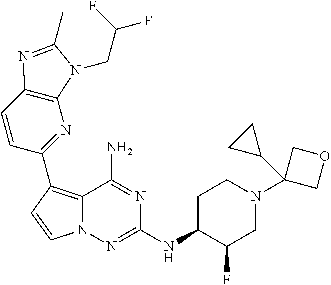 [SMILES: Cc1nc2ccc(-c3ccn4nc(N[C@H]5CCN(C6(C7CC7)COC6)C[C@H]5F)nc(N)c34)nc2n1CC(F)F] |
| 725 | 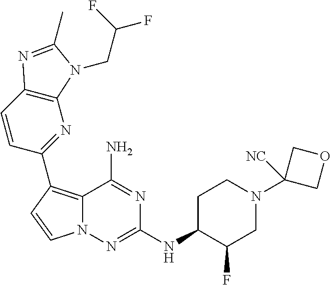 [SMILES: Cc1nc2ccc(-c3ccn4nc(N[C@H]5CCN(C6(C#N)COC6)C[C@H]5F)nc(N)c34)nc2n1CC(F)F] |
| 726 | 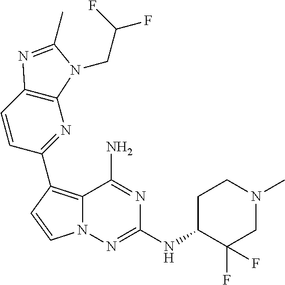 [SMILES: Cc1nc2ccc(-c3ccn4nc(N[C@@H]5CCN(C)CC5(F)F)nc(N)c34)nc2n1CC(F)F] |
| 727 | 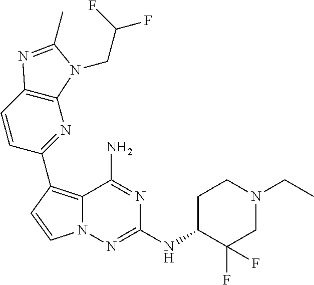 [SMILES: CCN1CC[C@@H](Nc2nc(N)c3c(-c4ccc5nc(C)n(CC(F)F)c5n4)ccn3n2)C(F)(F)C1] |
| 728 | 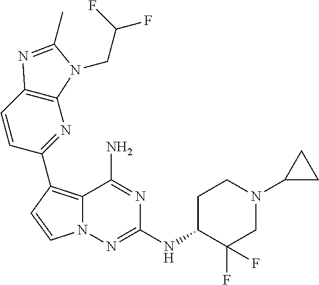 [SMILES: Cc1nc2ccc(-c3ccn4nc(N[C@@H]5CCN(C6CC6)CC5(F)F)nc(N)c34)nc2n1CC(F)F] |
| 729 | 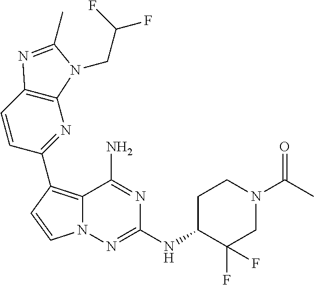 [SMILES: CC(=O)N1CC[C@@H](Nc2nc(N)c3c(-c4ccc5nc(C)n(CC(F)F)c5n4)ccn3n2)C(F)(F)C1] |
| 730 | 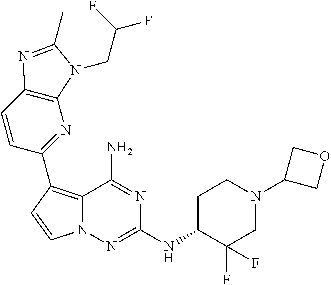 [SMILES: Cc1nc2ccc(-c3ccn4nc(N[C@@H]5CCN(C6COC6)CC5(F)F)nc(N)c34)nc2n1CC(F)F] |
| 731 | 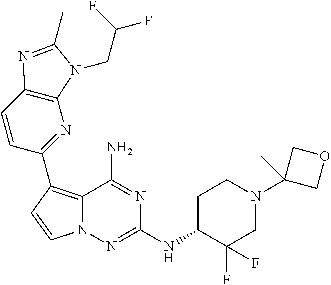 [SMILES: Cc1nc2ccc(-c3ccn4nc(N[C@@H]5CCN(C6(C)COC6)CC5(F)F)nc(N)c34)nc2n1CC(F)F] |
| 732 | 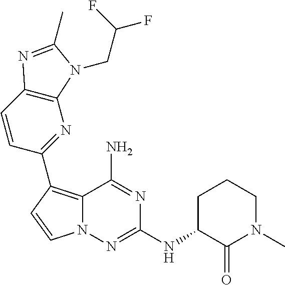 [SMILES: Cc1nc2ccc(-c3ccn4nc(N[C@@H]5CCCN(C)C5=O)nc(N)c34)nc2n1CC(F)F] |
| 733 | 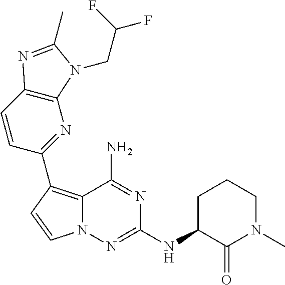 [SMILES: Cc1nc2ccc(-c3ccn4nc(N[C@H]5CCCN(C)C5=O)nc(N)c34)nc2n1CC(F)F] |
| 734 | 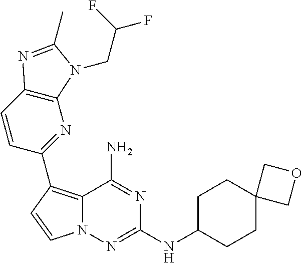 [SMILES: Cc1nc2ccc(-c3ccn4nc(NC5CCC6(CC5)COC6)nc(N)c34)nc2n1CC(F)F] |
| 735 | 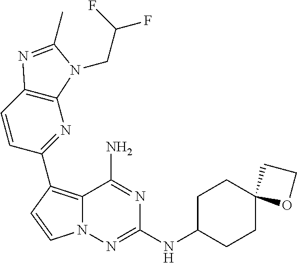 |
| 736 | 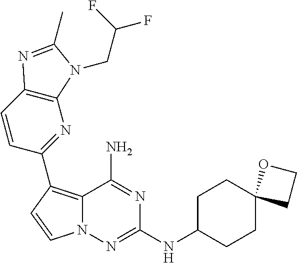 |
| 737 | 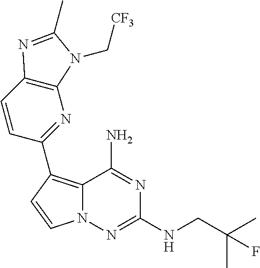 [SMILES: Cc1nc2ccc(-c3ccn4nc(NCC(C)(C)F)nc(N)c34)nc2n1CC(F)(F)F] |
| 738 | 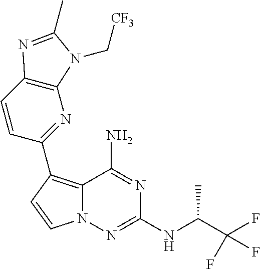 [SMILES: Cc1nc2ccc(-c3ccn4nc(N[C@H](C)C(F)(F)F)nc(N)c34)nc2n1CC(F)(F)F] |
| 739 | 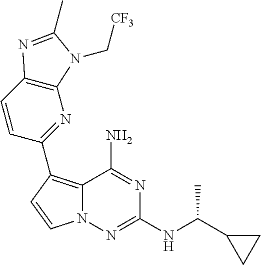 [SMILES: Cc1nc2ccc(-c3ccn4nc(N[C@H](C)C5CC5)nc(N)c34)nc2n1CC(F)(F)F] |
| 740 | 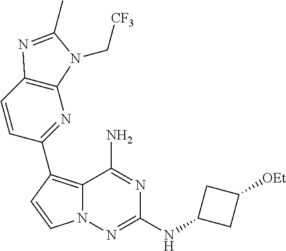 [SMILES: CCO[C@H]1C[C@@H](Nc2nc(N)c3c(-c4ccc5nc(C)n(CC(F)(F)F)c5n4)ccn3n2)C1] |
| 741 | 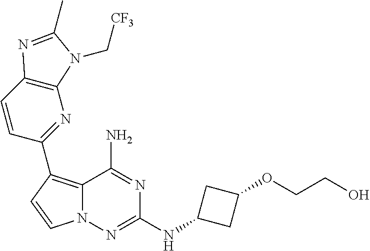 [SMILES: Cc1nc2ccc(-c3ccn4nc(N[C@H]5C[C@@H](OCCO)C5)nc(N)c34)nc2n1CC(F)(F)F] |
| 742 | 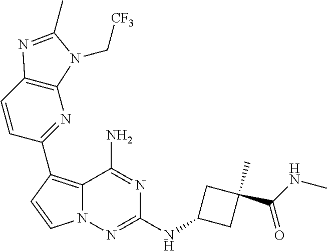 [SMILES: CNC(=O)[C@]1(C)C[C@@H](Nc2nc(N)c3c(-c4ccc5nc(C)n(CC(F)(F)F)c5n4)ccn3n2)C1] |
| 743 | 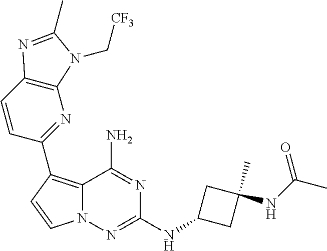 [SMILES: CC(=O)N[C@]1(C)C[C@@H](Nc2nc(N)c3c(-c4ccc5nc(C)n(CC(F)(F)F)c5n4)ccn3n2)C1] |
| 744 | 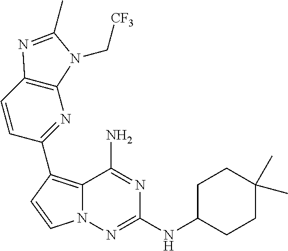 [SMILES: Cc1nc2ccc(-c3ccn4nc(NC5CCC(C)(C)CC5)nc(N)c34)nc2n1CC(F)(F)F] |
| 745 | 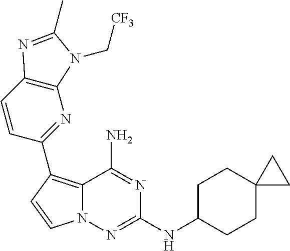 [SMILES: Cc1nc2ccc(-c3ccn4nc(NC5CCC6(CC5)CC6)nc(N)c34)nc2n1CC(F)(F)F] |
| 746 | 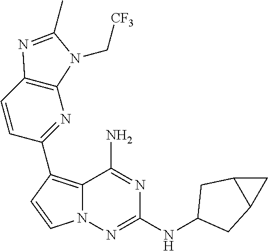 [SMILES: Cc1nc2ccc(-c3ccn4nc(NC5CC6CC6C5)nc(N)c34)nc2n1CC(F)(F)F] |
| 747 | 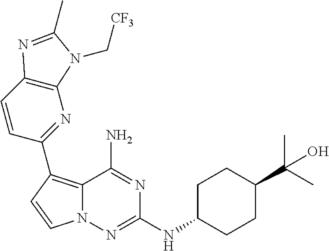 [SMILES: Cc1nc2ccc(-c3ccn4nc(N[C@H]5CC[C@H](C(C)(C)O)CC5)nc(N)c34)nc2n1CC(F)(F)F] |
| 748 | 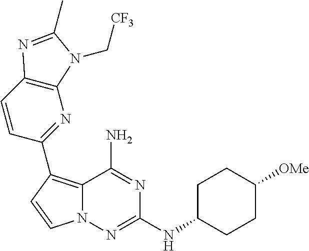 [SMILES: CO[C@H]1CC[C@@H](Nc2nc(N)c3c(-c4ccc5nc(C)n(CC(F)(F)F)c5n4)ccn3n2)CC1] |
| 749 | 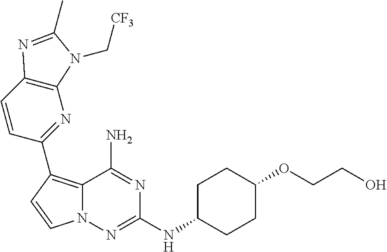 [SMILES: Cc1nc2ccc(-c3ccn4nc(N[C@H]5CC[C@@H](OCCO)CC5)nc(N)c34)nc2n1CC(F)(F)F] |
| 750 | 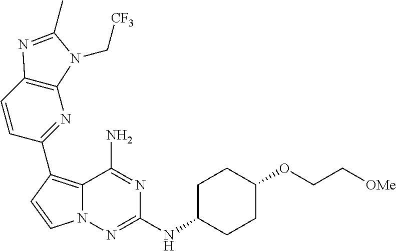 [SMILES: COCCO[C@H]1CC[C@@H](Nc2nc(N)c3c(-c4ccc5nc(C)n(CC(F)(F)F)c5n4)ccn3n2)CC1] |
| 751 | 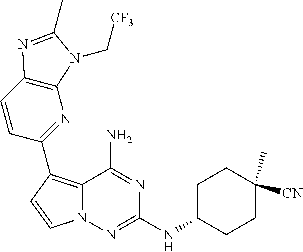 [SMILES: Cc1nc2ccc(-c3ccn4nc(N[C@H]5CC[C@](C)(C#N)CC5)nc(N)c34)nc2n1CC(F)(F)F] |
| 752 | 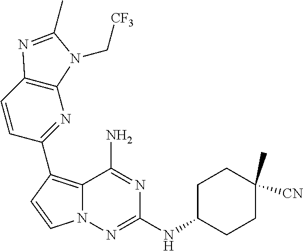 [SMILES: Cc1nc2ccc(-c3ccn4nc(N[C@H]5CC[C@@](C)(C#N)CC5)nc(N)c34)nc2n1CC(F)(F)F] |
| 753 | 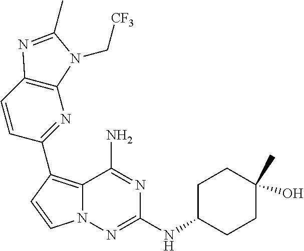 [SMILES: Cc1nc2ccc(-c3ccn4nc(N[C@H]5CC[C@@](C)(O)CC5)nc(N)c34)nc2n1CC(F)(F)F] |
| 754 | 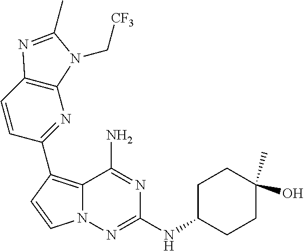 [SMILES: Cc1nc2ccc(-c3ccn4nc(N[C@H]5CC[C@](C)(O)CC5)nc(N)c34)nc2n1CC(F)(F)F] |
| 755 | 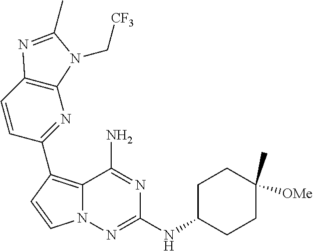 [SMILES: CO[C@]1(C)CC[C@H](Nc2nc(N)c3c(-c4ccc5nc(C)n(CC(F)(F)F)c5n4)ccn3n2)CC1] |
| 756 | 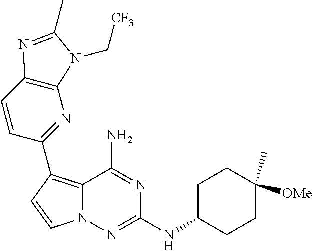 [SMILES: CO[C@]1(C)CC[C@@H](Nc2nc(N)c3c(-c4ccc5nc(C)n(CC(F)(F)F)c5n4)ccn3n2)CC1] |
| 757 | 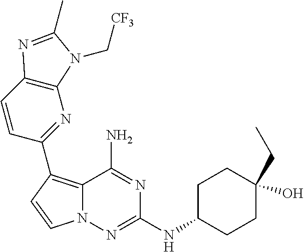 [SMILES: CC[C@]1(O)CC[C@@H](Nc2nc(N)c3c(-c4ccc5nc(C)n(CC(F)(F)F)c5n4)ccn3n2)CC1] |
| 758 | 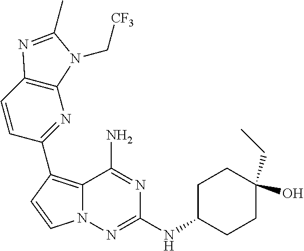 [SMILES: CC[C@]1(O)CC[C@H](Nc2nc(N)c3c(-c4ccc5nc(C)n(CC(F)(F)F)c5n4)ccn3n2)CC1] |
| 759 | 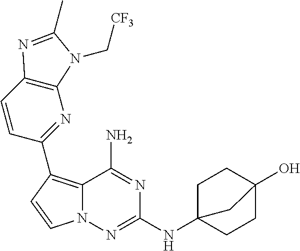 [SMILES: Cc1nc2ccc(-c3ccn4nc(NC56CCC(O)(CC5)C6)nc(N)c34)nc2n1CC(F)(F)F] |
| 760 | 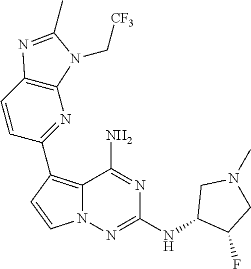 [SMILES: Cc1nc2ccc(-c3ccn4nc(N[C@@H]5CN(C)C[C@@H]5F)nc(N)c34)nc2n1CC(F)(F)F] |
| 761 | 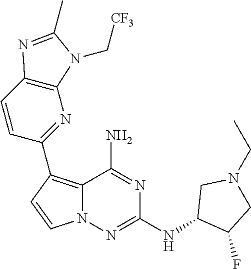 [SMILES: CCN1C[C@H](F)[C@H](Nc2nc(N)c3c(-c4ccc5nc(C)n(CC(F)(F)F)c5n4)ccn3n2)C1] |
| 762 | 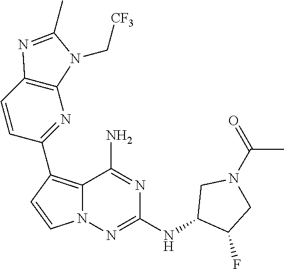 [SMILES: CC(=O)N1C[C@H](F)[C@H](Nc2nc(N)c3c(-c4ccc5nc(C)n(CC(F)(F)F)c5n4)ccn3n2)C1] |
| 763 | 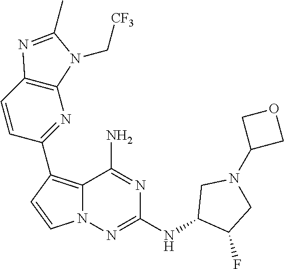 [SMILES: Cc1nc2ccc(-c3ccn4nc(N[C@@H]5CN(C6COC6)C[C@@H]5F)nc(N)c34)nc2n1CC(F)(F)F] |
| 764 | 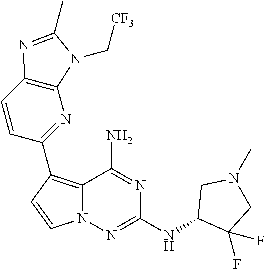 [SMILES: Cc1nc2ccc(-c3ccn4nc(N[C@@H]5CN(C)CC5(F)F)nc(N)c34)nc2n1CC(F)(F)F] |
| 765 | 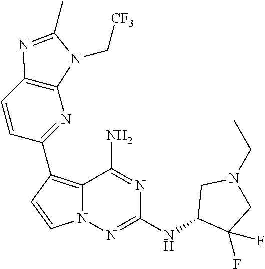 [SMILES: CCN1C[C@@H](Nc2nc(N)c3c(-c4ccc5nc(C)n(CC(F)(F)F)c5n4)ccn3n2)C(F)(F)C1] |
| 766 | 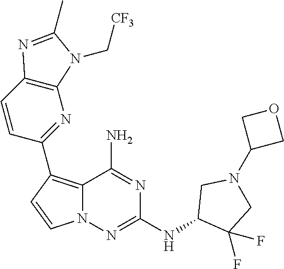 [SMILES: Cc1nc2ccc(-c3ccn4nc(N[C@@H]5CN(C6COC6)CC5(F)F)nc(N)c34)nc2n1CC(F)(F)F] |
| 767 | 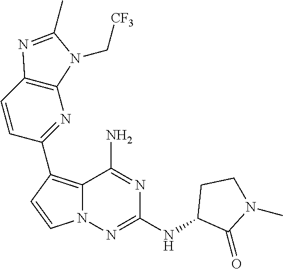 [SMILES: Cc1nc2ccc(-c3ccn4nc(N[C@@H]5CCN(C)C5=O)nc(N)c34)nc2n1CC(F)(F)F] |
| 768 | 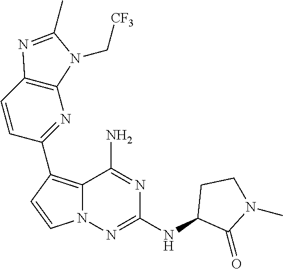 [SMILES: Cc1nc2ccc(-c3ccn4nc(N[C@H]5CCN(C)C5=O)nc(N)c34)nc2n1CC(F)(F)F] |
| 769 | 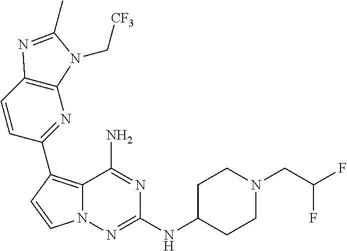 [SMILES: Cc1nc2ccc(-c3ccn4nc(NC5CCN(CC(F)F)CC5)nc(N)c34)nc2n1CC(F)(F)F] |
| 770 | 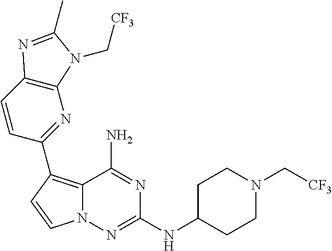 [SMILES: Cc1nc2ccc(-c3ccn4nc(NC5CCN(CC(F)(F)F)CC5)nc(N)c34)nc2n1CC(F)(F)F] |
| 771 | 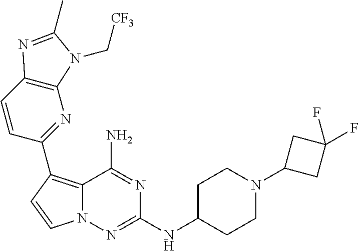 [SMILES: Cc1nc2ccc(-c3ccn4nc(NC5CCN(C6CC(F)(F)C6)CC5)nc(N)c34)nc2n1CC(F)(F)F] |
| 772 | 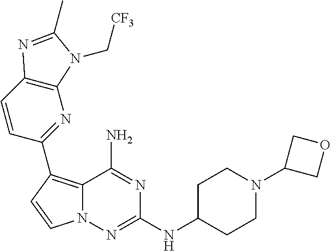 [SMILES: Cc1nc2ccc(-c3ccn4nc(NC5CCN(C6COC6)CC5)nc(N)c34)nc2n1CC(F)(F)F] |
| 773 | 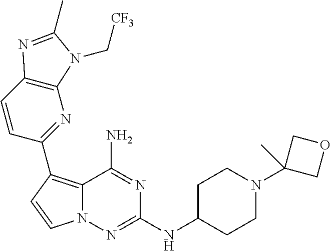 [SMILES: Cc1nc2ccc(-c3ccn4nc(NC5CCN(C6(C)COC6)CC5)nc(N)c34)nc2n1CC(F)(F)F] |
| 774 | 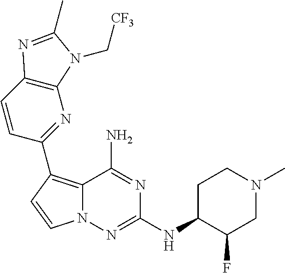 [SMILES: Cc1nc2ccc(-c3ccn4nc(N[C@H]5CCN(C)C[C@H]5F)nc(N)c34)nc2n1CC(F)(F)F] |
| 775 | 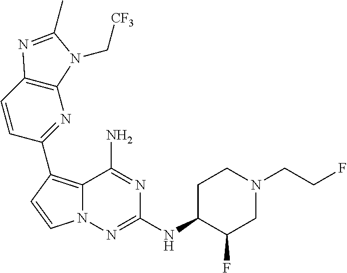 [SMILES: Cc1nc2ccc(-c3ccn4nc(N[C@H]5CCN(CCF)C[C@H]5F)nc(N)c34)nc2n1CC(F)(F)F] |
| 776 | 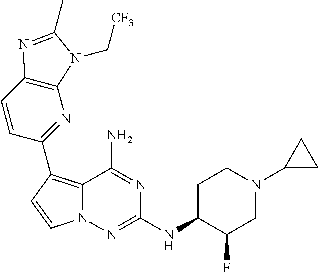 [SMILES: Cc1nc2ccc(-c3ccn4nc(N[C@H]5CCN(C6CC6)C[C@H]5F)nc(N)c34)nc2n1CC(F)(F)F] |
| 777 | 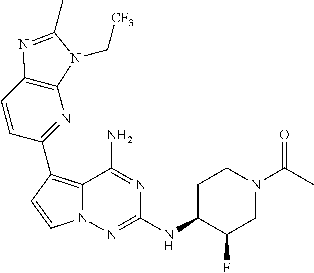 [SMILES: CC(=O)N1CC[C@H](Nc2nc(N)c3c(-c4ccc5nc(C)n(CC(F)(F)F)c5n4)ccn3n2)[C@H](F)C1] |
| 778 | 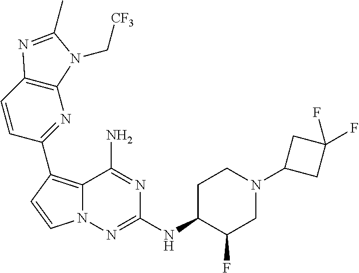 [SMILES: Cc1nc2ccc(-c3ccn4nc(N[C@H]5CCN(C6CC(F)(F)C6)C[C@H]5F)nc(N)c34)nc2n1CC(F)(F)F] |
| 779 | 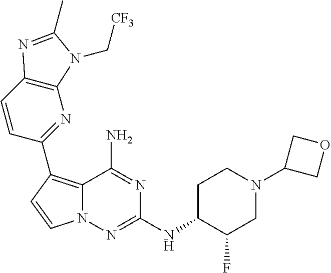 [SMILES: Cc1nc2ccc(-c3ccn4nc(N[C@@H]5CCN(C6COC6)C[C@@H]5F)nc(N)c34)nc2n1CC(F)(F)F] |
| 780 | 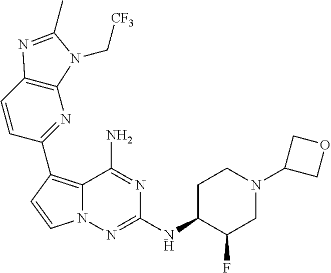 [SMILES: Cc1nc2ccc(-c3ccn4nc(N[C@H]5CCN(C6COC6)C[C@H]5F)nc(N)c34)nc2n1CC(F)(F)F] |
| 781 | 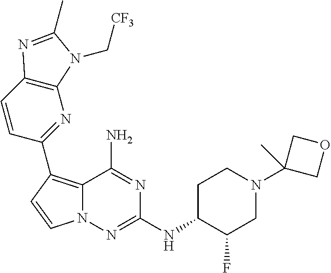 [SMILES: Cc1nc2ccc(-c3ccn4nc(N[C@@H]5CCN(C6(C)COC6)C[C@@H]5F)nc(N)c34)nc2n1CC(F)(F)F] |
| 782 | 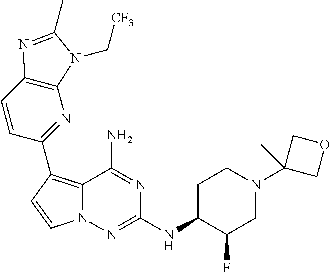 [SMILES: Cc1nc2ccc(-c3ccn4nc(N[C@H]5CCN(C6(C)COC6)C[C@H]5F)nc(N)c34)nc2n1CC(F)(F)F] |
| 783 | 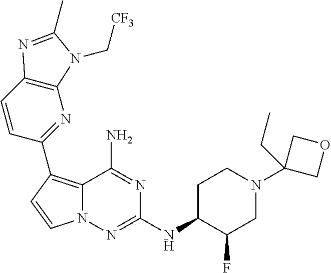 [SMILES: CCC1(N2CC[C@H](Nc3nc(N)c4c(-c5ccc6nc(C)n(CC(F)(F)F)c6n5)ccn4n3)[C@H](F)C2)COC1] |
| 784 | 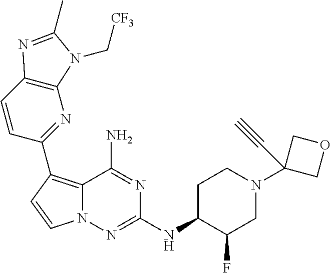 [SMILES: C#CC1(N2CC[C@H](Nc3nc(N)c4c(-c5ccc6nc(C)n(CC(F)(F)F)c6n5)ccn4n3)[C@H](F)C2)COC1] |
| 785 | 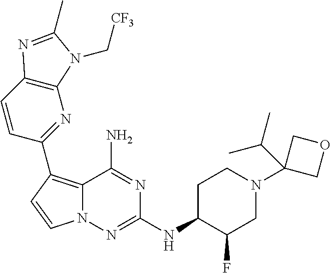 [SMILES: Cc1nc2ccc(-c3ccn4nc(N[C@H]5CCN(C6(C(C)C)COC6)C[C@H]5F)nc(N)c34)nc2n1CC(F)(F)F] |
| 786 | 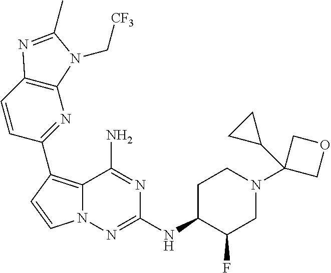 [SMILES: Cc1nc2ccc(-c3ccn4nc(N[C@H]5CCN(C6(C7CC7)COC6)C[C@H]5F)nc(N)c34)nc2n1CC(F)(F)F] |
| 787 | 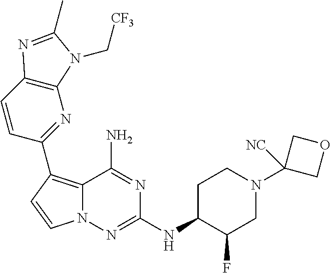 [SMILES: Cc1nc2ccc(-c3ccn4nc(N[C@H]5CCN(C6(C#N)COC6)C[C@H]5F)nc(N)c34)nc2n1CC(F)(F)F] |
| 788 | 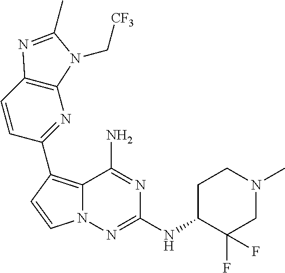 [SMILES: Cc1nc2ccc(-c3ccn4nc(N[C@@H]5CCN(C)CC5(F)F)nc(N)c34)nc2n1CC(F)(F)F] |
| 789 | 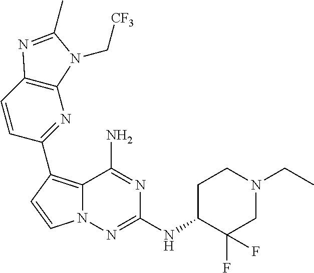 [SMILES: CCN1CC[C@@H](Nc2nc(N)c3c(-c4ccc5nc(C)n(CC(F)(F)F)c5n4)ccn3n2)C(F)(F)C1] |
| 790 | 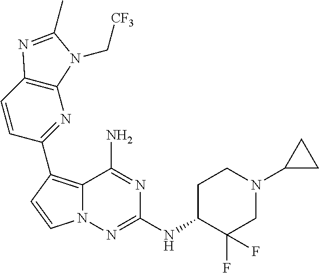 [SMILES: Cc1nc2ccc(-c3ccn4nc(N[C@@H]5CCN(C6CC6)CC5(F)F)nc(N)c34)nc2n1CC(F)(F)F] |
| 791 | 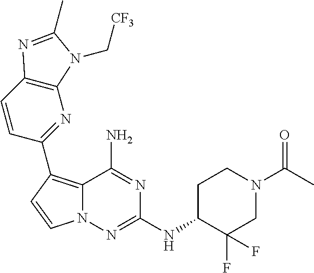 [SMILES: CC(=O)N1CC[C@@H](Nc2nc(N)c3c(-c4ccc5nc(C)n(CC(F)(F)F)c5n4)ccn3n2)C(F)(F)C1] |
| 792 | 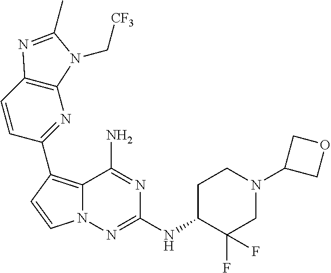 [SMILES: Cc1nc2ccc(-c3ccn4nc(N[C@@H]5CCN(C6COC6)CC5(F)F)nc(N)c34)nc2n1CC(F)(F)F] |
| 793 | 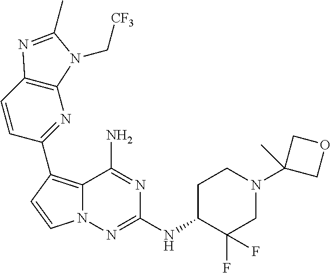 [SMILES: Cc1nc2ccc(-c3ccn4nc(N[C@@H]5CCN(C6(C)COC6)CC5(F)F)nc(N)c34)nc2n1CC(F)(F)F] |
| 794 | 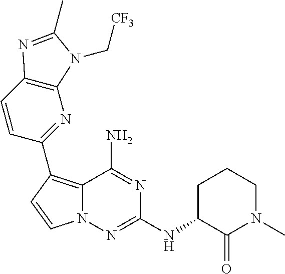 [SMILES: Cc1nc2ccc(-c3ccn4nc(N[C@@H]5CCCN(C)C5=O)nc(N)c34)nc2n1CC(F)(F)F] |
| 795 | 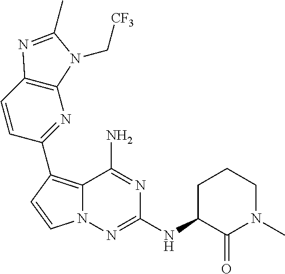 [SMILES: Cc1nc2ccc(-c3ccn4nc(N[C@H]5CCCN(C)C5=O)nc(N)c34)nc2n1CC(F)(F)F] |
| 796 | 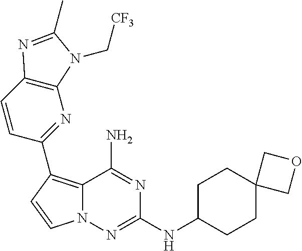 [SMILES: Cc1nc2ccc(-c3ccn4nc(NC5CCC6(CC5)COC6)nc(N)c34)nc2n1CC(F)(F)F] |
| 797 | 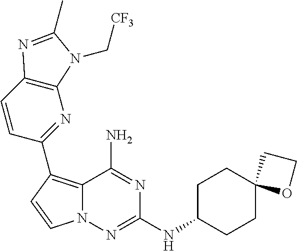 [SMILES: Cc1nc2ccc(-c3ccn4nc(N[C@H]5CC[C@@]6(CCO6)CC5)nc(N)c34)nc2n1CC(F)(F)F] |
| 798 | 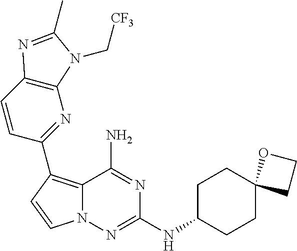 [SMILES: Cc1nc2ccc(-c3ccn4nc(N[C@H]5CC[C@]6(CCO6)CC5)nc(N)c34)nc2n1CC(F)(F)F] |
| 799 | 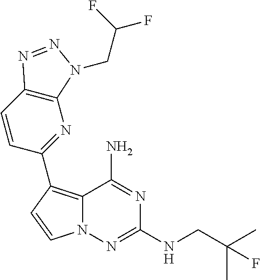 [SMILES: CC(C)(F)CNc1nc(N)c2c(-c3ccc4nnn(CC(F)F)c4n3)ccn2n1] |
| 800 | 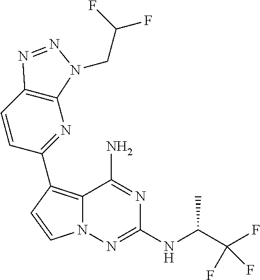 [SMILES: C[C@@H](Nc1nc(N)c2c(-c3ccc4nnn(CC(F)F)c4n3)ccn2n1)C(F)(F)F] |
| 801 | 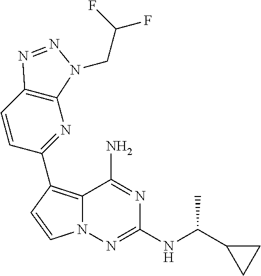 [SMILES: C[C@@H](Nc1nc(N)c2c(-c3ccc4nnn(CC(F)F)c4n3)ccn2n1)C1CC1] |
| 802 | 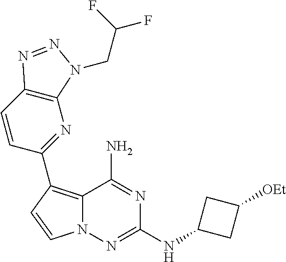 [SMILES: CCO[C@H]1C[C@@H](Nc2nc(N)c3c(-c4ccc5nnn(CC(F)F)c5n4)ccn3n2)C1] |
| 803 | 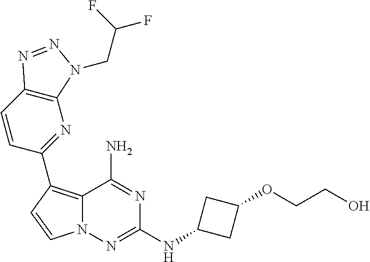 [SMILES: Nc1nc(N[C@H]2C[C@@H](OCCO)C2)nn2ccc(-c3ccc4nnn(CC(F)F)c4n3)c12] |
| 804 | 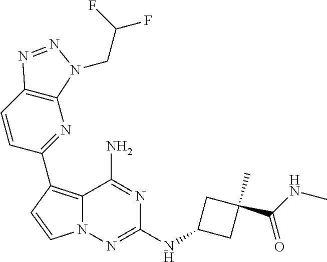 [SMILES: CNC(=O)[C@]1(C)C[C@@H](Nc2nc(N)c3c(-c4ccc5nnn(CC(F)F)c5n4)ccn3n2)C1] |
| 805 | 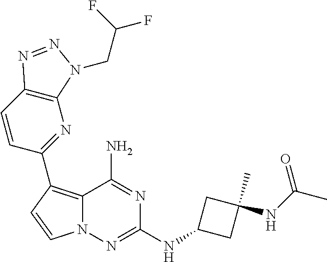 [SMILES: CC(=O)N[C@]1(C)C[C@@H](Nc2nc(N)c3c(-c4ccc5nnn(CC(F)F)c5n4)ccn3n2)C1] |
| 806 | 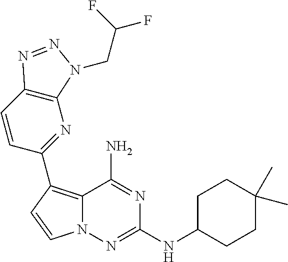 [SMILES: CC1(C)CCC(Nc2nc(N)c3c(-c4ccc5nnn(CC(F)F)c5n4)ccn3n2)CC1] |
| 807 | 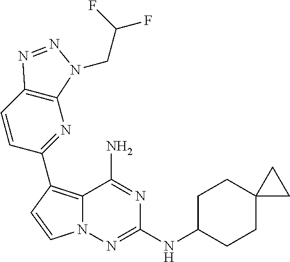 [SMILES: Nc1nc(NC2CCC3(CC2)CC3)nn2ccc(-c3ccc4nnn(CC(F)F)c4n3)c12] |
| 808 | 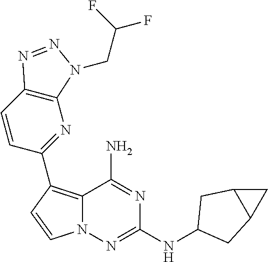 [SMILES: Nc1nc(NC2CC3CC3C2)nn2ccc(-c3ccc4nnn(CC(F)F)c4n3)c12] |
| 809 | 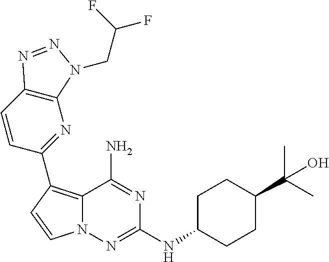 [SMILES: CC(C)(O)[C@H]1CC[C@H](Nc2nc(N)c3c(-c4ccc5nnn(CC(F)F)c5n4)ccn3n2)CC1] |
| 810 | 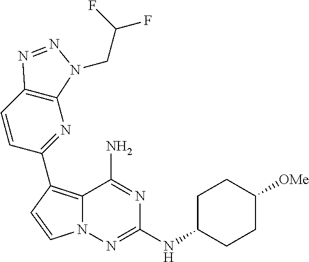 [SMILES: CO[C@H]1CC[C@@H](Nc2nc(N)c3c(-c4ccc5nnn(CC(F)F)c5n4)ccn3n2)CC1] |
| 811 | 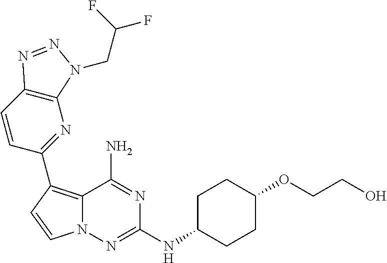 [SMILES: Nc1nc(N[C@H]2CC[C@@H](OCCO)CC2)nn2ccc(-c3ccc4nnn(CC(F)F)c4n3)c12] |
| 812 | 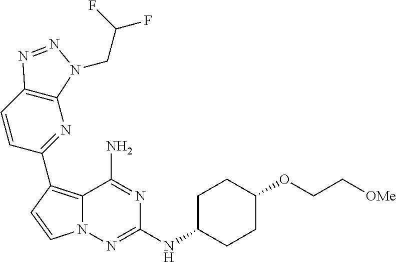 [SMILES: COCCO[C@H]1CC[C@@H](Nc2nc(N)c3c(-c4ccc5nnn(CC(F)F)c5n4)ccn3n2)CC1] |
| 813 | 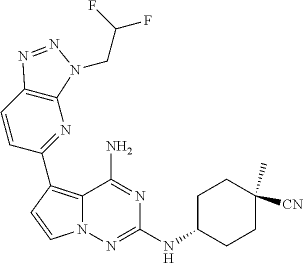 [SMILES: C[C@]1(C#N)CC[C@H](Nc2nc(N)c3c(-c4ccc5nnn(CC(F)F)c5n4)ccn3n2)CC1] |
| 814 | 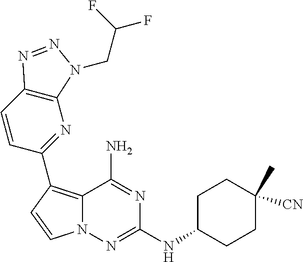 [SMILES: C[C@]1(C#N)CC[C@@H](Nc2nc(N)c3c(-c4ccc5nnn(CC(F)F)c5n4)ccn3n2)CC1] |
| 815 | 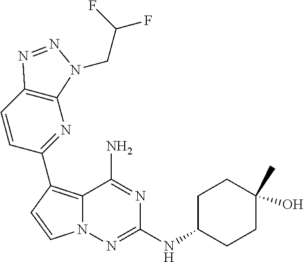 [SMILES: C[C@]1(O)CC[C@@H](Nc2nc(N)c3c(-c4ccc5nnn(CC(F)F)c5n4)ccn3n2)CC1] |
| 816 | 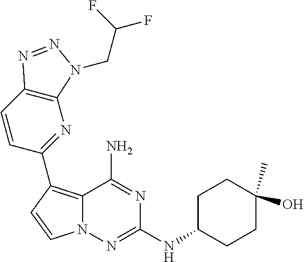 [SMILES: C[C@]1(O)CC[C@H](Nc2nc(N)c3c(-c4ccc5nnn(CC(F)F)c5n4)ccn3n2)CC1] |
| 817 | 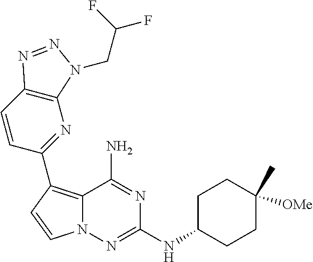 [SMILES: CO[C@]1(C)CC[C@H](Nc2nc(N)c3c(-c4ccc5nnn(CC(F)F)c5n4)ccn3n2)CC1] |
| 818 | 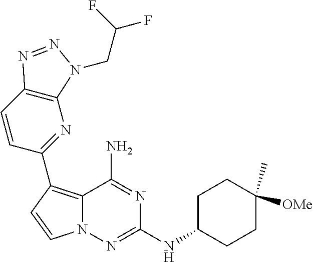 [SMILES: CO[C@]1(C)CC[C@@H](Nc2nc(N)c3c(-c4ccc5nnn(CC(F)F)c5n4)ccn3n2)CC1] |
| 819 | 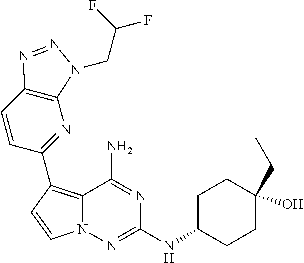 [SMILES: CC[C@]1(O)CC[C@@H](Nc2nc(N)c3c(-c4ccc5nnn(CC(F)F)c5n4)ccn3n2)CC1] |
| 820 | 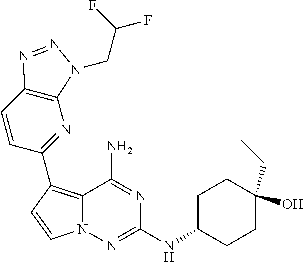 [SMILES: CC[C@]1(O)CC[C@H](Nc2nc(N)c3c(-c4ccc5nnn(CC(F)F)c5n4)ccn3n2)CC1] |
| 821 | 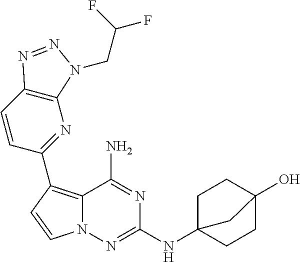 [SMILES: Nc1nc(NC23CCC(O)(CC2)C3)nn2ccc(-c3ccc4nnn(CC(F)F)c4n3)c12] |
| 822 | 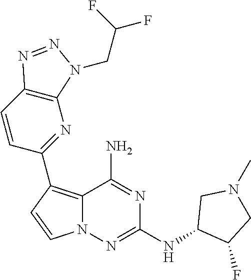 [SMILES: CN1C[C@H](F)[C@H](Nc2nc(N)c3c(-c4ccc5nnn(CC(F)F)c5n4)ccn3n2)C1] |
| 823 | 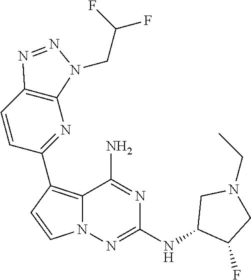 [SMILES: CCN1C[C@H](F)[C@H](Nc2nc(N)c3c(-c4ccc5nnn(CC(F)F)c5n4)ccn3n2)C1] |
| 824 | 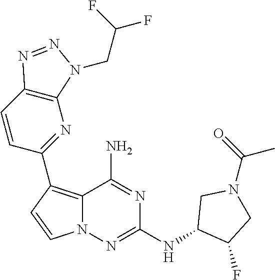 [SMILES: CC(=O)N1C[C@H](F)[C@H](Nc2nc(N)c3c(-c4ccc5nnn(CC(F)F)c5n4)ccn3n2)C1] |
| 825 | 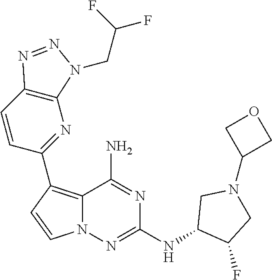 [SMILES: Nc1nc(N[C@@H]2CN(C3COC3)C[C@@H]2F)nn2ccc(-c3ccc4nnn(CC(F)F)c4n3)c12] |
| 826 | 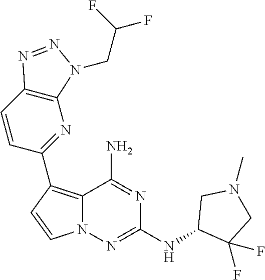 [SMILES: CN1C[C@@H](Nc2nc(N)c3c(-c4ccc5nnn(CC(F)F)c5n4)ccn3n2)C(F)(F)C1] |
| 827 | 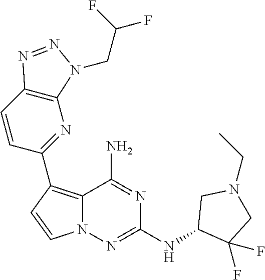 [SMILES: CCN1C[C@@H](Nc2nc(N)c3c(-c4ccc5nnn(CC(F)F)c5n4)ccn3n2)C(F)(F)C1] |
| 828 | 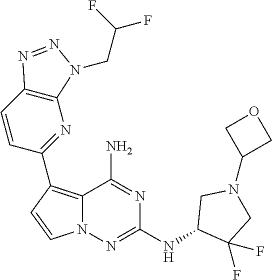 [SMILES: Nc1nc(N[C@@H]2CN(C3COC3)CC2(F)F)nn2ccc(-c3ccc4nnn(CC(F)F)c4n3)c12] |
| 829 | 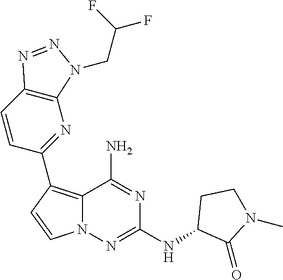 [SMILES: CN1CC[C@@H](Nc2nc(N)c3c(-c4ccc5nnn(CC(F)F)c5n4)ccn3n2)C1=O] |
| 830 | 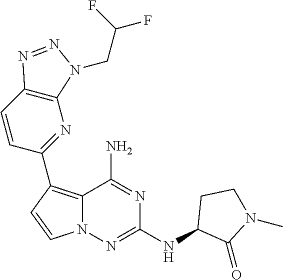 [SMILES: CN1CC[C@H](Nc2nc(N)c3c(-c4ccc5nnn(CC(F)F)c5n4)ccn3n2)C1=O] |
| 831 | 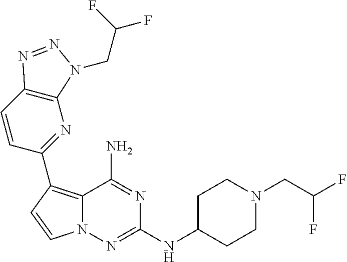 [SMILES: Nc1nc(NC2CCN(CC(F)F)CC2)nn2ccc(-c3ccc4nnn(CC(F)F)c4n3)c12] |
| 832 | 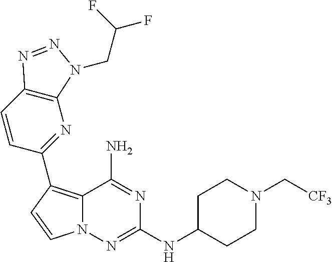 [SMILES: Nc1nc(NC2CCN(CC(F)(F)F)CC2)nn2ccc(-c3ccc4nnn(CC(F)F)c4n3)c12] |
| 833 | 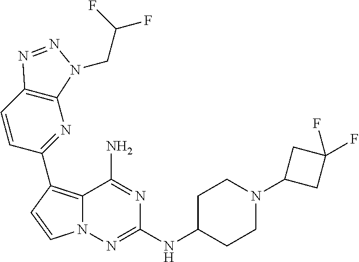 [SMILES: Nc1nc(NC2CCN(C3CC(F)(F)C3)CC2)nn2ccc(-c3ccc4nnn(CC(F)F)c4n3)c12] |
| 834 | 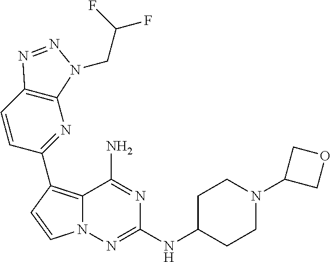 [SMILES: Nc1nc(NC2CCN(C3COC3)CC2)nn2ccc(-c3ccc4nnn(CC(F)F)c4n3)c12] |
| 835 | 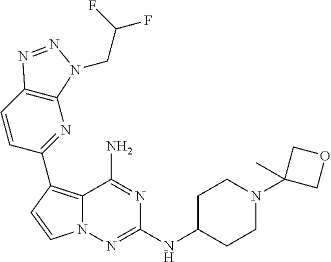 [SMILES: CC1(N2CCC(Nc3nc(N)c4c(-c5ccc6nnn(CC(F)F)c6n5)ccn4n3)CC2)COC1] |
| 836 | 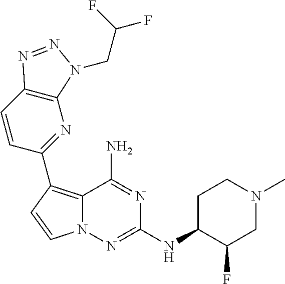 [SMILES: CN1CC[C@H](Nc2nc(N)c3c(-c4ccc5nnn(CC(F)F)c5n4)ccn3n2)[C@H](F)C1] |
| 837 | 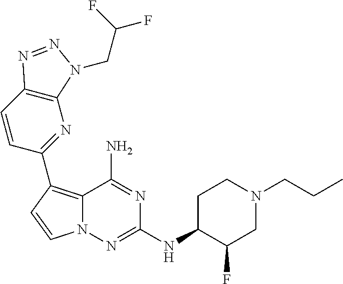 [SMILES: CCCN1CC[C@H](Nc2nc(N)c3c(-c4ccc5nnn(CC(F)F)c5n4)ccn3n2)[C@H](F)C1] |
| 838 | 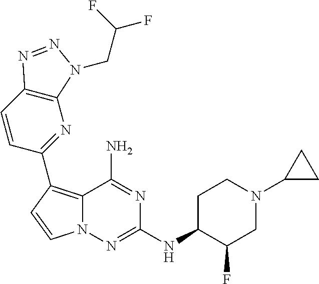 [SMILES: Nc1nc(N[C@H]2CCN(C3CC3)C[C@H]2F)nn2ccc(-c3ccc4nnn(CC(F)F)c4n3)c12] |
| 839 | 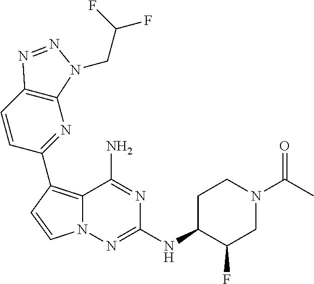 [SMILES: CC(=O)N1CC[C@H](Nc2nc(N)c3c(-c4ccc5nnn(CC(F)F)c5n4)ccn3n2)[C@H](F)C1] |
| 840 | 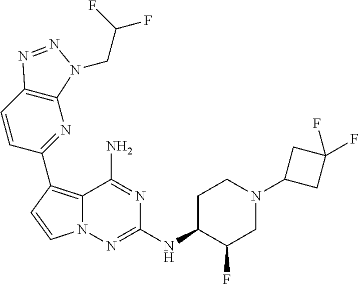 [SMILES: Nc1nc(N[C@H]2CCN(C3CC(F)(F)C3)C[C@H]2F)nn2ccc(-c3ccc4nnn(CC(F)F)c4n3)c12] |
| 841 | 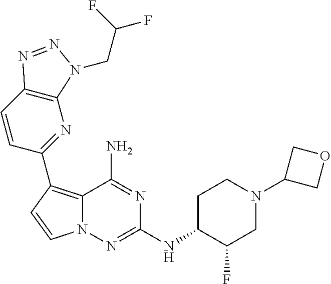 [SMILES: Nc1nc(N[C@@H]2CCN(C3COC3)C[C@@H]2F)nn2ccc(-c3ccc4nnn(CC(F)F)c4n3)c12] |
| 842 | 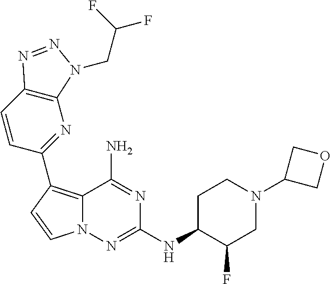 [SMILES: Nc1nc(N[C@H]2CCN(C3COC3)C[C@H]2F)nn2ccc(-c3ccc4nnn(CC(F)F)c4n3)c12] |
| 843 | 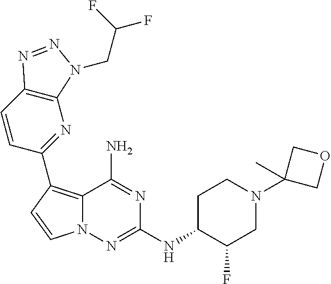 [SMILES: CC1(N2CC[C@@H](Nc3nc(N)c4c(-c5ccc6nnn(CC(F)F)c6n5)ccn4n3)[C@@H](F)C2)COC1] |
| 844 | 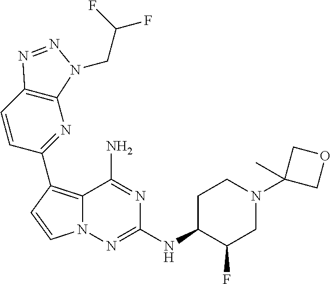 [SMILES: CC1(N2CC[C@H](Nc3nc(N)c4c(-c5ccc6nnn(CC(F)F)c6n5)ccn4n3)[C@H](F)C2)COC1] |
| 845 | 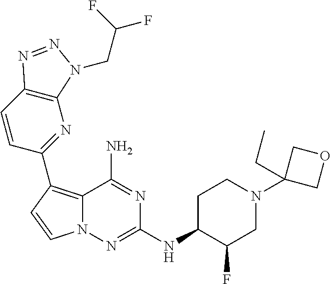 [SMILES: CCC1(N2CC[C@H](Nc3nc(N)c4c(-c5ccc6nnn(CC(F)F)c6n5)ccn4n3)[C@H](F)C2)COC1] |
| 846 | 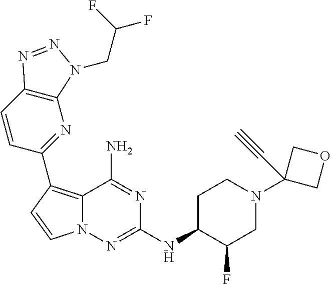 [SMILES: C#CC1(N2CC[C@H](Nc3nc(N)c4c(-c5ccc6nnn(CC(F)F)c6n5)ccn4n3)[C@H](F)C2)COC1] |
| 847 | 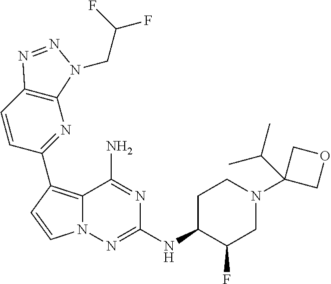 [SMILES: CC(C)C1(N2CC[C@H](Nc3nc(N)c4c(-c5ccc6nnn(CC(F)F)c6n5)ccn4n3)[C@H](F)C2)COC1] |
| 848 | 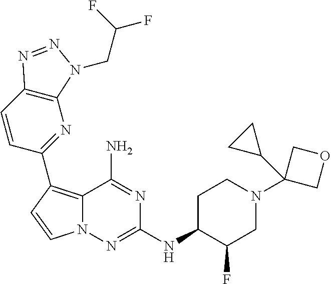 [SMILES: Nc1nc(N[C@H]2CCN(C3(C4CC4)COC3)C[C@H]2F)nn2ccc(-c3ccc4nnn(CC(F)F)c4n3)c12] |
| 849 | 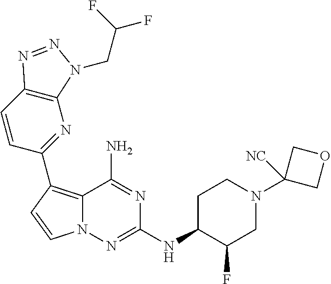 [SMILES: N#CC1(N2CC[C@H](Nc3nc(N)c4c(-c5ccc6nnn(CC(F)F)c6n5)ccn4n3)[C@H](F)C2)COC1] |
| 850 | 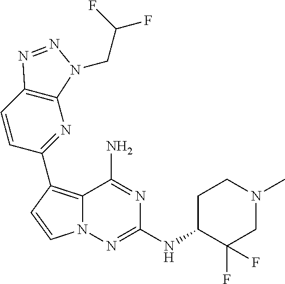 [SMILES: CN1CC[C@@H](Nc2nc(N)c3c(-c4ccc5nnn(CC(F)F)c5n4)ccn3n2)C(F)(F)C1] |
| 851 | 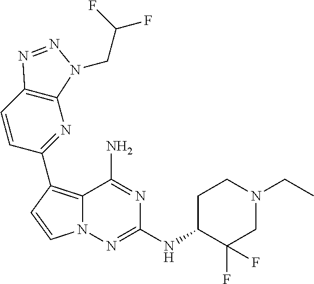 [SMILES: CCN1CC[C@@H](Nc2nc(N)c3c(-c4ccc5nnn(CC(F)F)c5n4)ccn3n2)C(F)(F)C1] |
| 852 | 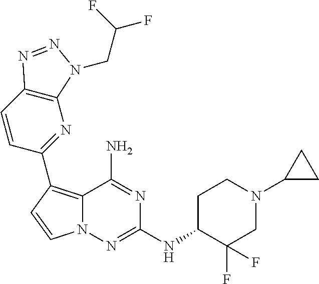 [SMILES: Nc1nc(N[C@@H]2CCN(C3CC3)CC2(F)F)nn2ccc(-c3ccc4nnn(CC(F)F)c4n3)c12] |
| 853 | 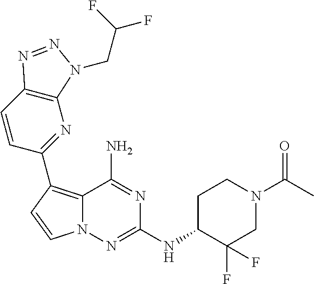 [SMILES: CC(=O)N1CC[C@@H](Nc2nc(N)c3c(-c4ccc5nnn(CC(F)F)c5n4)ccn3n2)C(F)(F)C1] |
| 854 | 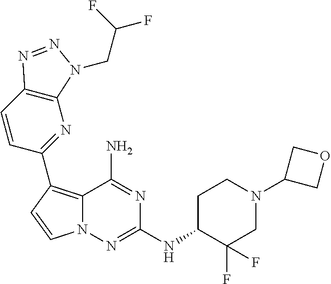 [SMILES: Nc1nc(N[C@@H]2CCN(C3COC3)CC2(F)F)nn2ccc(-c3ccc4nnn(CC(F)F)c4n3)c12] |
| 855 | 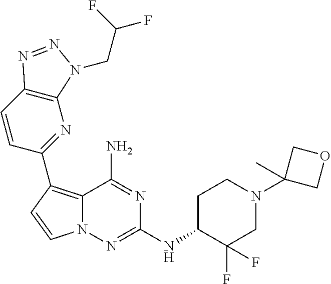 [SMILES: CC1(N2CC[C@@H](Nc3nc(N)c4c(-c5ccc6nnn(CC(F)F)c6n5)ccn4n3)C(F)(F)C2)COC1] |
| 856 | 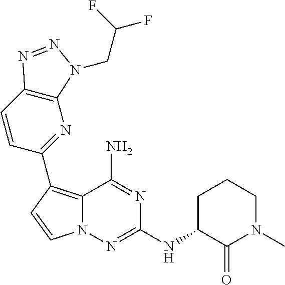 [SMILES: CN1CCC[C@@H](Nc2nc(N)c3c(-c4ccc5nnn(CC(F)F)c5n4)ccn3n2)C1=O] |
| 857 | 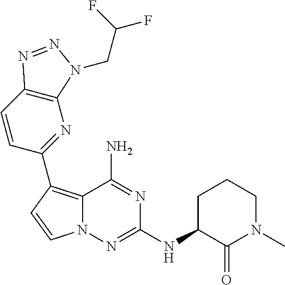 [SMILES: CN1CCC[C@H](Nc2nc(N)c3c(-c4ccc5nnn(CC(F)F)c5n4)ccn3n2)C1=O] |
| 858 | 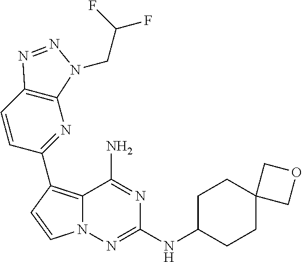 [SMILES: Nc1nc(NC2CCC3(CC2)COC3)nn2ccc(-c3ccc4nnn(CC(F)F)c4n3)c12] |
| 859 | 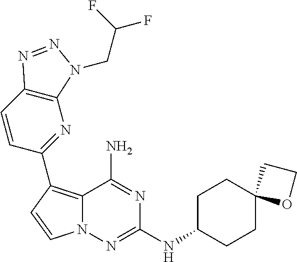 [SMILES: Nc1nc(N[C@H]2CC[C@@]3(CCO3)CC2)nn2ccc(-c3ccc4nnn(CC(F)F)c4n3)c12] |
| 860 | 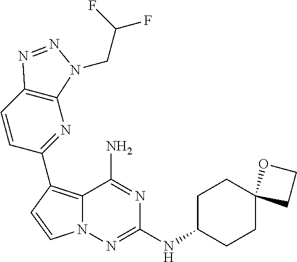 [SMILES: Nc1nc(N[C@H]2CC[C@]3(CCO3)CC2)nn2ccc(-c3ccc4nnn(CC(F)F)c4n3)c12] |
| 861 | 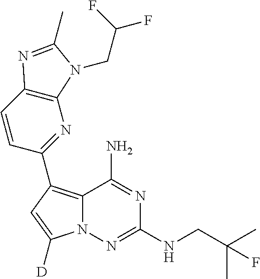 [SMILES: [2H]c1cc(-c2ccc3nc(C)n(CC(F)F)c3n2)c2c(N)nc(NCC(C)(C)F)nn12] |
| 862 | 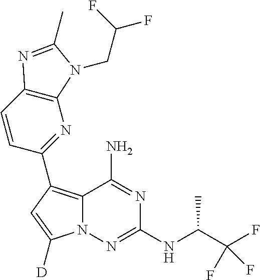 [SMILES: [2H]c1cc(-c2ccc3nc(C)n(CC(F)F)c3n2)c2c(N)nc(N[C@H](C)C(F)(F)F)nn12] |
| 863 | 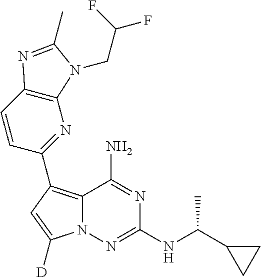 [SMILES: [2H]c1cc(-c2ccc3nc(C)n(CC(F)F)c3n2)c2c(N)nc(N[C@H](C)C3CC3)nn12] |
| 864 | 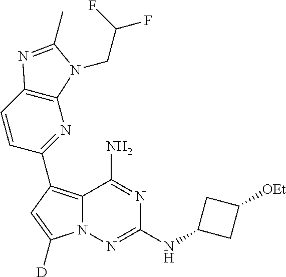 [SMILES: [2H]c1cc(-c2ccc3nc(C)n(CC(F)F)c3n2)c2c(N)nc(N[C@H]3C[C@@H](OCC)C3)nn12] |
| 865 | 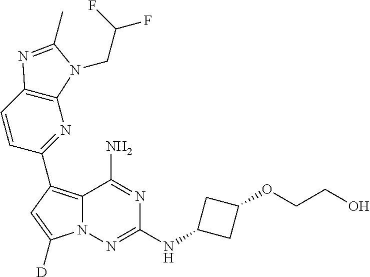 [SMILES: [2H]c1cc(-c2ccc3nc(C)n(CC(F)F)c3n2)c2c(N)nc(N[C@H]3C[C@@H](OCCO)C3)nn12] |
| 866 | 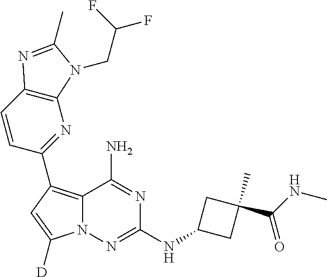 [SMILES: [2H]c1cc(-c2ccc3nc(C)n(CC(F)F)c3n2)c2c(N)nc(N[C@H]3C[C@](C)(C(=O)NC)C3)nn12] |
| 867 | 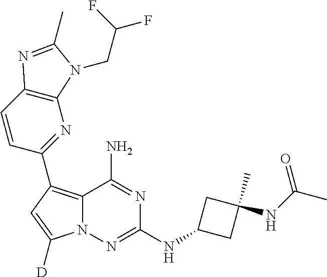 [SMILES: [2H]c1cc(-c2ccc3nc(C)n(CC(F)F)c3n2)c2c(N)nc(N[C@H]3C[C@](C)(NC(C)=O)C3)nn12] |
| 868 | 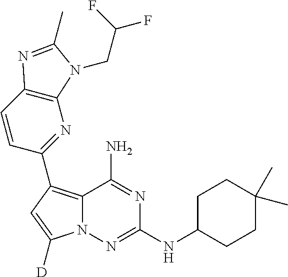 [SMILES: [2H]c1cc(-c2ccc3nc(C)n(CC(F)F)c3n2)c2c(N)nc(NC3CCC(C)(C)CC3)nn12] |
| 869 | 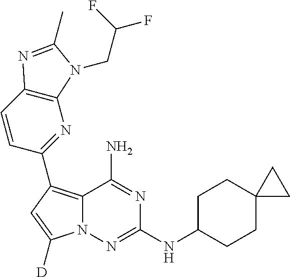 [SMILES: [2H]c1cc(-c2ccc3nc(C)n(CC(F)F)c3n2)c2c(N)nc(NC3CCC4(CC3)CC4)nn12] |
| 870 | 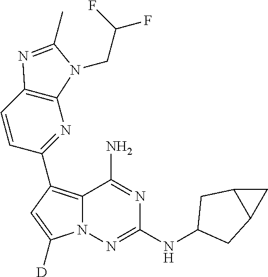 [SMILES: [2H]c1cc(-c2ccc3nc(C)n(CC(F)F)c3n2)c2c(N)nc(NC3CC4CC4C3)nn12] |
| 871 | 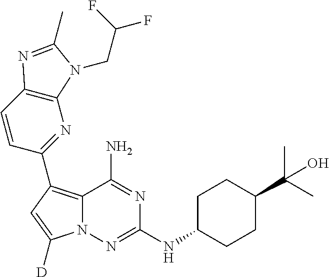 [SMILES: [2H]c1cc(-c2ccc3nc(C)n(CC(F)F)c3n2)c2c(N)nc(N[C@H]3CC[C@H](C(C)(C)O)CC3)nn12] |
| 872 | 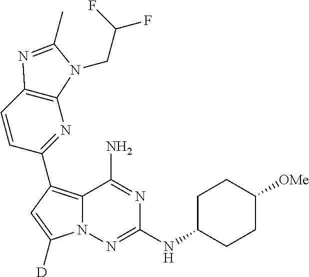 [SMILES: [2H]c1cc(-c2ccc3nc(C)n(CC(F)F)c3n2)c2c(N)nc(N[C@H]3CC[C@@H](OC)CC3)nn12] |
| 873 | 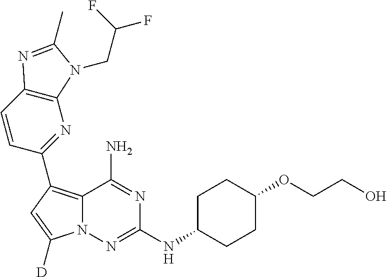 [SMILES: [2H]c1cc(-c2ccc3nc(C)n(CC(F)F)c3n2)c2c(N)nc(N[C@H]3CC[C@@H](OCCO)CC3)nn12] |
| 874 | 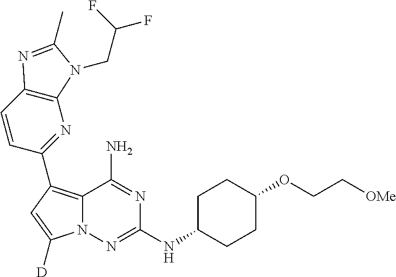 [SMILES: [2H]c1cc(-c2ccc3nc(C)n(CC(F)F)c3n2)c2c(N)nc(N[C@H]3CC[C@@H](OCCOC)CC3)nn12] |
| 875 | 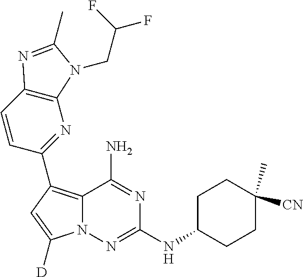 [SMILES: [2H]c1cc(-c2ccc3nc(C)n(CC(F)F)c3n2)c2c(N)nc(N[C@H]3CC[C@](C)(C#N)CC3)nn12] |
| 876 | 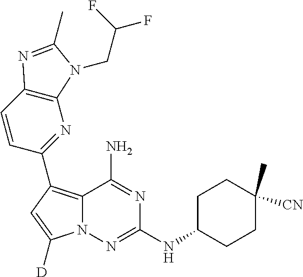 [SMILES: [2H]c1cc(-c2ccc3nc(C)n(CC(F)F)c3n2)c2c(N)nc(N[C@H]3CC[C@@](C)(C#N)CC3)nn12] |
| 877 | 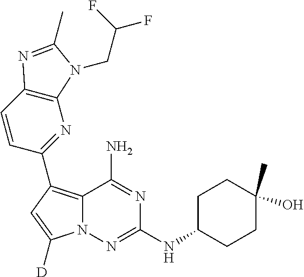 [SMILES: [2H]c1cc(-c2ccc3nc(C)n(CC(F)F)c3n2)c2c(N)nc(N[C@H]3CC[C@@](C)(O)CC3)nn12] |
| 878 | 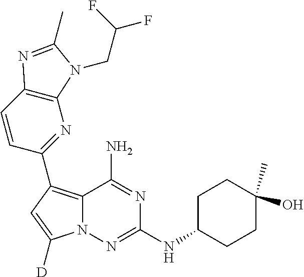 [SMILES: [2H]c1cc(-c2ccc3nc(C)n(CC(F)F)c3n2)c2c(N)nc(N[C@H]3CC[C@](C)(O)CC3)nn12] |
| 879 | 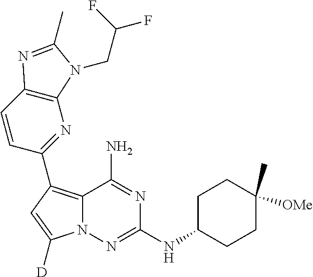 [SMILES: [2H]c1cc(-c2ccc3nc(C)n(CC(F)F)c3n2)c2c(N)nc(N[C@H]3CC[C@@](C)(OC)CC3)nn12] |
| 880 | 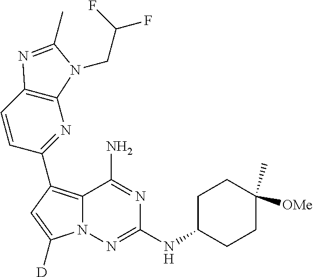 [SMILES: [2H]c1cc(-c2ccc3nc(C)n(CC(F)F)c3n2)c2c(N)nc(N[C@H]3CC[C@](C)(OC)CC3)nn12] |
| 881 | 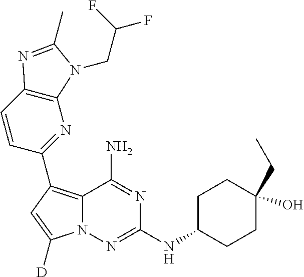 [SMILES: [2H]c1cc(-c2ccc3nc(C)n(CC(F)F)c3n2)c2c(N)nc(N[C@H]3CC[C@](O)(CC)CC3)nn12] |
| 882 | 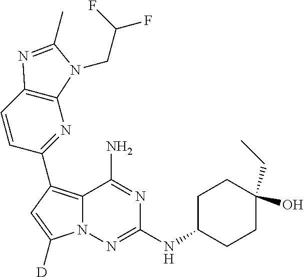 [SMILES: [2H]c1cc(-c2ccc3nc(C)n(CC(F)F)c3n2)c2c(N)nc(N[C@H]3CC[C@@](O)(CC)CC3)nn12] |
| 883 | 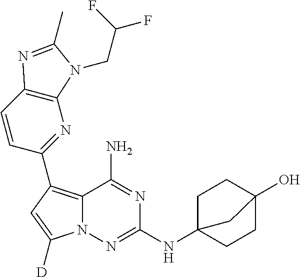 [SMILES: [2H]c1cc(-c2ccc3nc(C)n(CC(F)F)c3n2)c2c(N)nc(NC34CCC(O)(CC3)C4)nn12] |
| 884 | 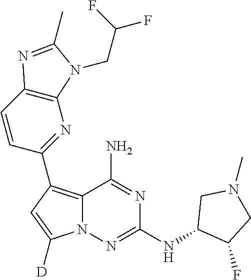 [SMILES: [2H]c1cc(-c2ccc3nc(C)n(CC(F)F)c3n2)c2c(N)nc(N[C@@H]3CN(C)C[C@@H]3F)nn12] |
| 885 | 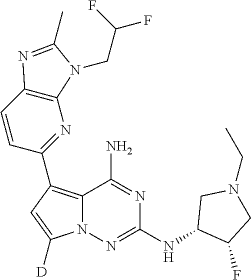 [SMILES: [2H]c1cc(-c2ccc3nc(C)n(CC(F)F)c3n2)c2c(N)nc(N[C@@H]3CN(CC)C[C@@H]3F)nn12] |
| 886 | 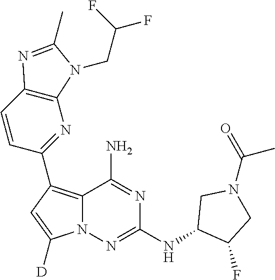 [SMILES: [2H]c1cc(-c2ccc3nc(C)n(CC(F)F)c3n2)c2c(N)nc(N[C@@H]3CN(C(C)=O)C[C@@H]3F)nn12] |
| 887 | 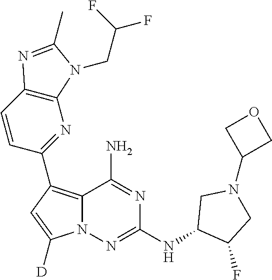 [SMILES: [2H]c1cc(-c2ccc3nc(C)n(CC(F)F)c3n2)c2c(N)nc(N[C@@H]3CN(C4COC4)C[C@@H]3F)nn12] |
| 888 | 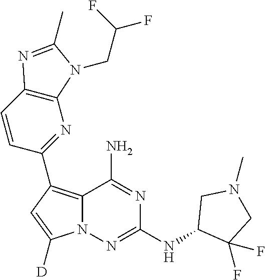 [SMILES: [2H]c1cc(-c2ccc3nc(C)n(CC(F)F)c3n2)c2c(N)nc(N[C@@H]3CN(C)CC3(F)F)nn12] |
| 889 | 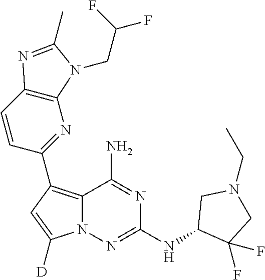 [SMILES: [2H]c1cc(-c2ccc3nc(C)n(CC(F)F)c3n2)c2c(N)nc(N[C@@H]3CN(CC)CC3(F)F)nn12] |
| 890 | 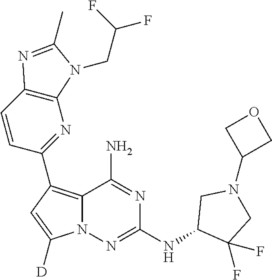 [SMILES: [2H]c1cc(-c2ccc3nc(C)n(CC(F)F)c3n2)c2c(N)nc(N[C@@H]3CN(C4COC4)CC3(F)F)nn12] |
| 891 | 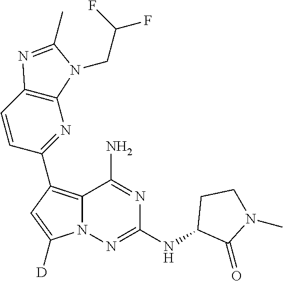 [SMILES: [2H]c1cc(-c2ccc3nc(C)n(CC(F)F)c3n2)c2c(N)nc(N[C@@H]3CCN(C)C3=O)nn12] |
| 892 | 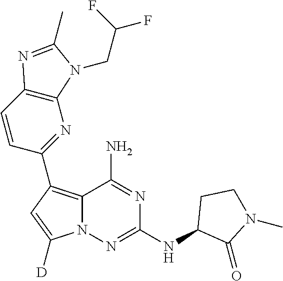 [SMILES: [2H]c1cc(-c2ccc3nc(C)n(CC(F)F)c3n2)c2c(N)nc(N[C@H]3CCN(C)C3=O)nn12] |
| 893 | 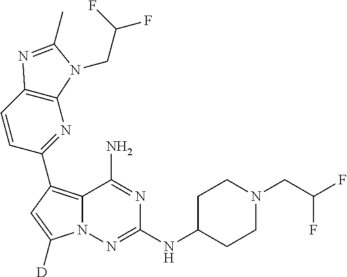 [SMILES: [2H]c1cc(-c2ccc3nc(C)n(CC(F)F)c3n2)c2c(N)nc(NC3CCN(CC(F)F)CC3)nn12] |
| 894 | 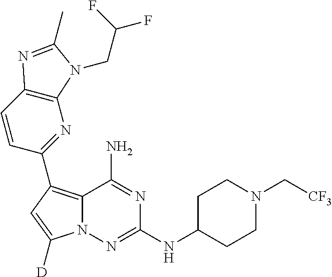 [SMILES: [2H]c1cc(-c2ccc3nc(C)n(CC(F)F)c3n2)c2c(N)nc(NC3CCN(CC(F)(F)F)CC3)nn12] |
| 895 | 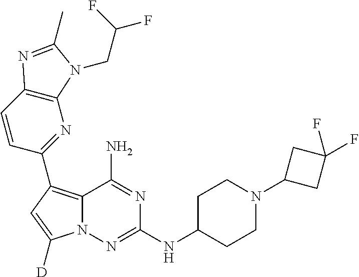 [SMILES: [2H]c1cc(-c2ccc3nc(C)n(CC(F)F)c3n2)c2c(N)nc(NC3CCN(C4CC(F)(F)C4)CC3)nn12] |
| 896 | 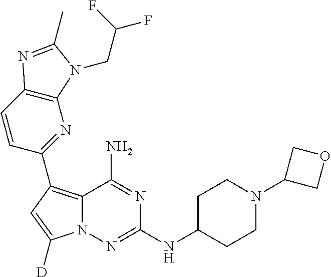 [SMILES: [2H]c1cc(-c2ccc3nc(C)n(CC(F)F)c3n2)c2c(N)nc(NC3CCN(C4COC4)CC3)nn12] |
| 897 | 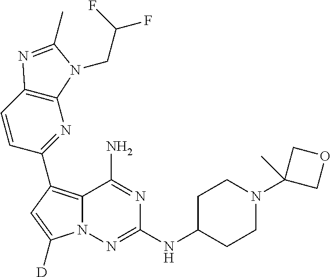 [SMILES: [2H]c1cc(-c2ccc3nc(C)n(CC(F)F)c3n2)c2c(N)nc(NC3CCN(C4(C)COC4)CC3)nn12] |
| 898 | 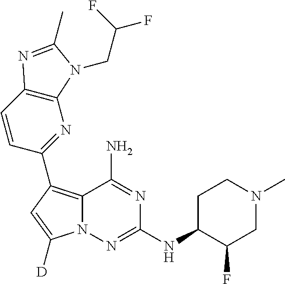 [SMILES: [2H]c1cc(-c2ccc3nc(C)n(CC(F)F)c3n2)c2c(N)nc(N[C@H]3CCN(C)C[C@H]3F)nn12] |
| 899 | 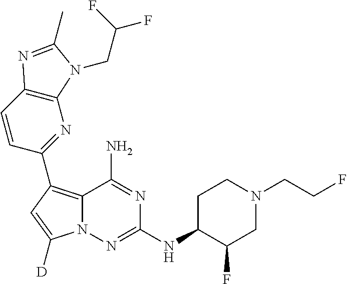 [SMILES: [2H]c1cc(-c2ccc3nc(C)n(CC(F)F)c3n2)c2c(N)nc(N[C@H]3CCN(CCF)C[C@H]3F)nn12] |
| 900 | 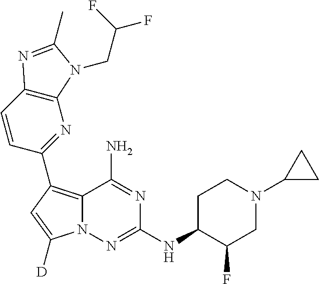 [SMILES: [2H]c1cc(-c2ccc3nc(C)n(CC(F)F)c3n2)c2c(N)nc(N[C@H]3CCN(C4CC4)C[C@H]3F)nn12] |
| 901 | 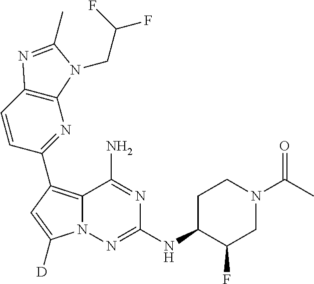 [SMILES: [2H]c1cc(-c2ccc3nc(C)n(CC(F)F)c3n2)c2c(N)nc(N[C@H]3CCN(C(C)=O)C[C@H]3F)nn12] |
| 902 | 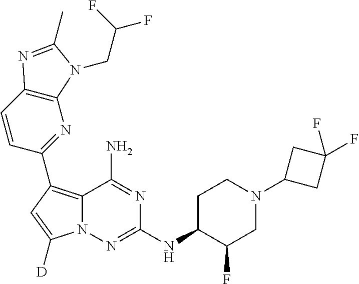 [SMILES: [2H]c1cc(-c2ccc3nc(C)n(CC(F)F)c3n2)c2c(N)nc(N[C@H]3CCN(C4CC(F)(F)C4)C[C@H]3F)nn12] |
| 903 | 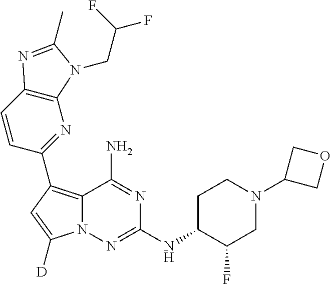 [SMILES: [2H]c1cc(-c2ccc3nc(C)n(CC(F)F)c3n2)c2c(N)nc(N[C@@H]3CCN(C4COC4)C[C@@H]3F)nn12] |
| 904 | 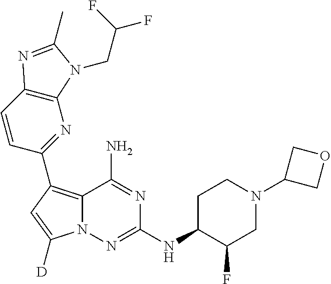 [SMILES: [2H]c1cc(-c2ccc3nc(C)n(CC(F)F)c3n2)c2c(N)nc(N[C@H]3CCN(C4COC4)C[C@H]3F)nn12] |
| 905 | 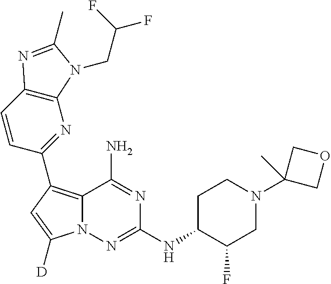 [SMILES: [2H]c1cc(-c2ccc3nc(C)n(CC(F)F)c3n2)c2c(N)nc(N[C@@H]3CCN(C4(C)COC4)C[C@@H]3F)nn12] |
| 906 | 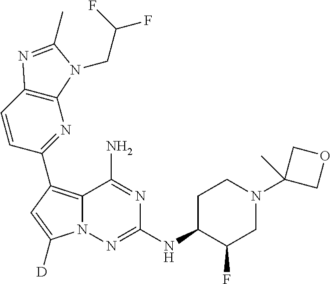 [SMILES: [2H]c1cc(-c2ccc3nc(C)n(CC(F)F)c3n2)c2c(N)nc(N[C@H]3CCN(C4(C)COC4)C[C@H]3F)nn12] |
| 907 | 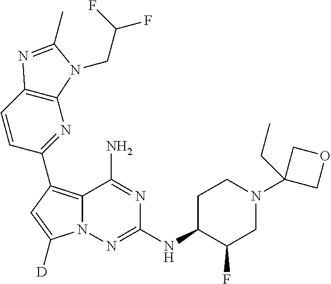 [SMILES: [2H]c1cc(-c2ccc3nc(C)n(CC(F)F)c3n2)c2c(N)nc(N[C@H]3CCN(C4(CC)COC4)C[C@H]3F)nn12] |
| 908 | 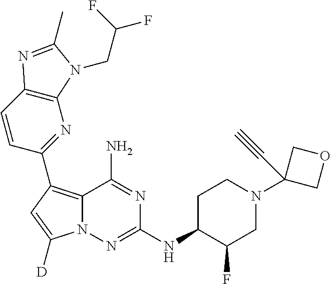 [SMILES: [2H]c1cc(-c2ccc3nc(C)n(CC(F)F)c3n2)c2c(N)nc(N[C@H]3CCN(C4(C#C)COC4)C[C@H]3F)nn12] |
| 909 | 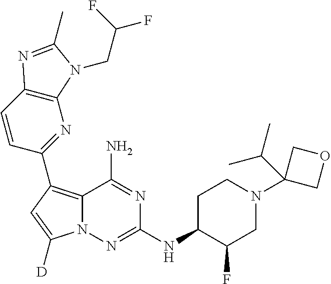 [SMILES: [2H]c1cc(-c2ccc3nc(C)n(CC(F)F)c3n2)c2c(N)nc(N[C@H]3CCN(C4(C(C)C)COC4)C[C@H]3F)nn12] |
| 910 | 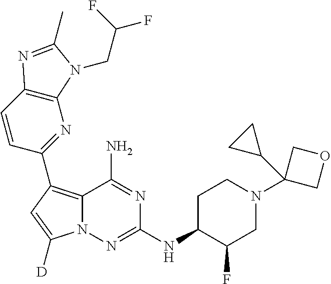 [SMILES: [2H]c1cc(-c2ccc3nc(C)n(CC(F)F)c3n2)c2c(N)nc(N[C@H]3CCN(C4(C5CC5)COC4)C[C@H]3F)nn12] |
| 911 | 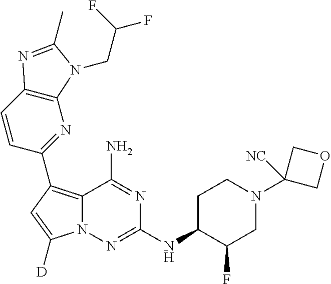 [SMILES: [2H]c1cc(-c2ccc3nc(C)n(CC(F)F)c3n2)c2c(N)nc(N[C@H]3CCN(C4(C#N)COC4)C[C@H]3F)nn12] |
| 912 | 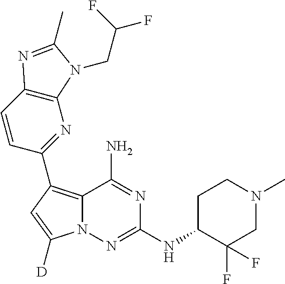 [SMILES: [2H]c1cc(-c2ccc3nc(C)n(CC(F)F)c3n2)c2c(N)nc(N[C@@H]3CCN(C)CC3(F)F)nn12] |
| 913 | 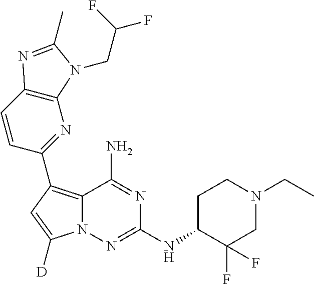 [SMILES: [2H]c1cc(-c2ccc3nc(C)n(CC(F)F)c3n2)c2c(N)nc(N[C@@H]3CCN(CC)CC3(F)F)nn12] |
| 914 | 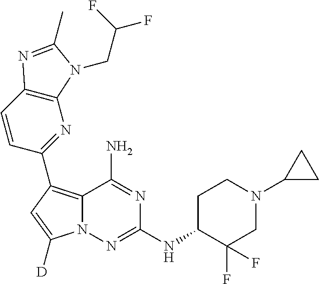 [SMILES: [2H]c1cc(-c2ccc3nc(C)n(CC(F)F)c3n2)c2c(N)nc(N[C@@H]3CCN(C4CC4)CC3(F)F)nn12] |
| 915 | 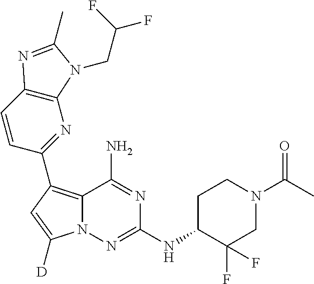 [SMILES: [2H]c1cc(-c2ccc3nc(C)n(CC(F)F)c3n2)c2c(N)nc(N[C@@H]3CCN(C(C)=O)CC3(F)F)nn12] |
| 916 | 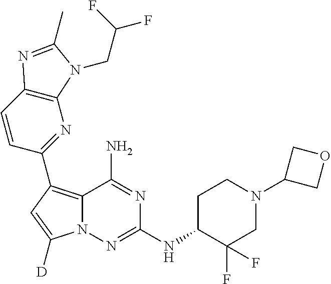 [SMILES: [2H]c1cc(-c2ccc3nc(C)n(CC(F)F)c3n2)c2c(N)nc(N[C@@H]3CCN(C4COC4)CC3(F)F)nn12] |
| 917 | 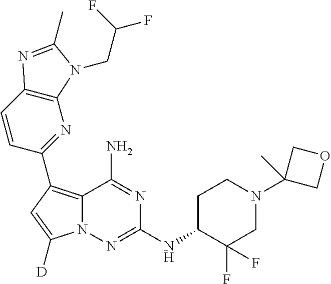 [SMILES: [2H]c1cc(-c2ccc3nc(C)n(CC(F)F)c3n2)c2c(N)nc(N[C@@H]3CCN(C4(C)COC4)CC3(F)F)nn12] |
| 918 | 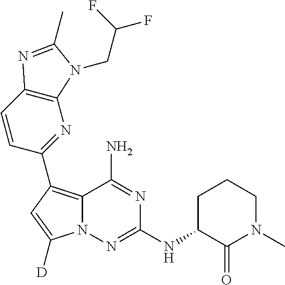 [SMILES: [2H]c1cc(-c2ccc3nc(C)n(CC(F)F)c3n2)c2c(N)nc(N[C@@H]3CCCN(C)C3=O)nn12] |
| 919 | 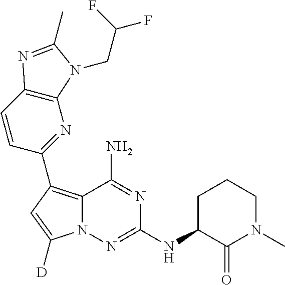 [SMILES: [2H]c1cc(-c2ccc3nc(C)n(CC(F)F)c3n2)c2c(N)nc(N[C@H]3CCCN(C)C3=O)nn12] |
| 920 | 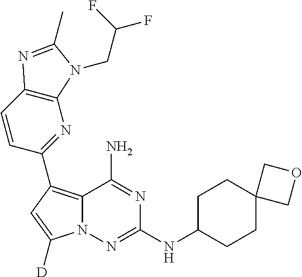 [SMILES: [2H]c1cc(-c2ccc3nc(C)n(CC(F)F)c3n2)c2c(N)nc(NC3CCC4(CC3)COC4)nn12] |
| 921 | 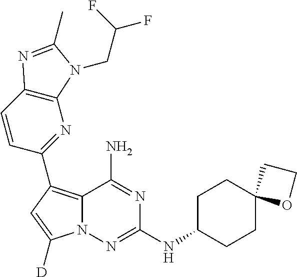 [SMILES: [2H]c1cc(-c2ccc3nc(C)n(CC(F)F)c3n2)c2c(N)nc(N[C@H]3CC[C@@]4(CCO4)CC3)nn12] |
| 922 | 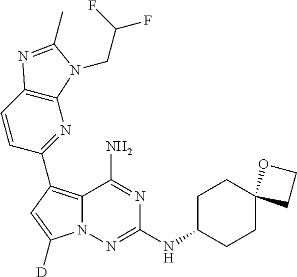 [SMILES: [2H]c1cc(-c2ccc3nc(C)n(CC(F)F)c3n2)c2c(N)nc(N[C@H]3CC[C@]4(CCO4)CC3)nn12] |
| 923 | 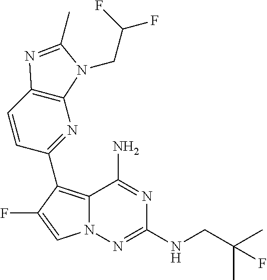 [SMILES: Cc1nc2ccc(-c3c(F)cn4nc(NCC(C)(C)F)nc(N)c34)nc2n1CC(F)F] |
| 924 | 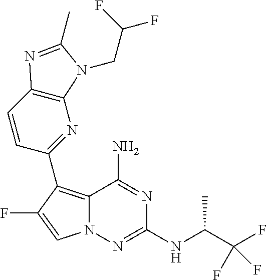 [SMILES: Cc1nc2ccc(-c3c(F)cn4nc(N[C@H](C)C(F)(F)F)nc(N)c34)nc2n1CC(F)F] |
| 925 | 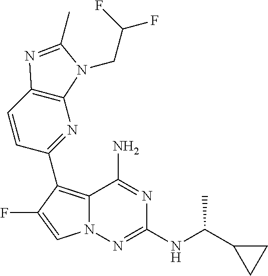 [SMILES: Cc1nc2ccc(-c3c(F)cn4nc(N[C@H](C)C5CC5)nc(N)c34)nc2n1CC(F)F] |
| 926 | 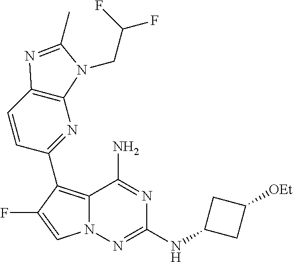 [SMILES: CCO[C@H]1C[C@@H](Nc2nc(N)c3c(-c4ccc5nc(C)n(CC(F)F)c5n4)c(F)cn3n2)C1] |
| 927 | 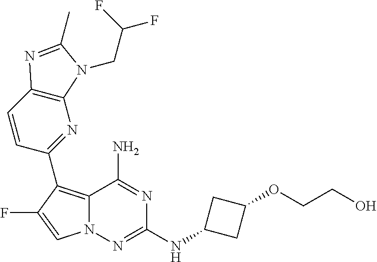 [SMILES: Cc1nc2ccc(-c3c(F)cn4nc(N[C@H]5C[C@@H](OCCO)C5)nc(N)c34)nc2n1CC(F)F] |
| 928 | 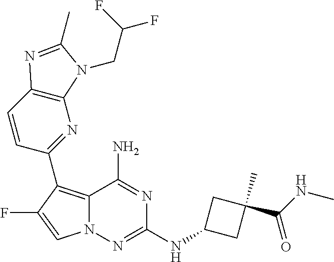 [SMILES: CNC(=O)[C@]1(C)C[C@@H](Nc2nc(N)c3c(-c4ccc5nc(C)n(CC(F)F)c5n4)c(F)cn3n2)C1] |
| 929 | 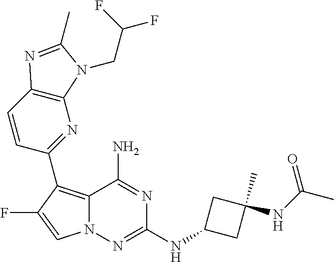 [SMILES: CC(=O)N[C@]1(C)C[C@@H](Nc2nc(N)c3c(-c4ccc5nc(C)n(CC(F)F)c5n4)c(F)cn3n2)C1] |
| 930 | 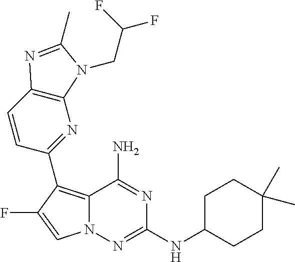 [SMILES: Cc1nc2ccc(-c3c(F)cn4nc(NC5CCC(C)(C)CC5)nc(N)c34)nc2n1CC(F)F] |
| 931 | 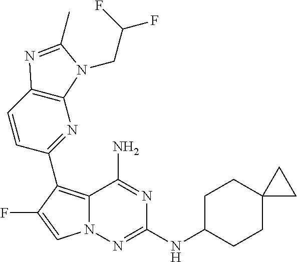 [SMILES: Cc1nc2ccc(-c3c(F)cn4nc(NC5CCC6(CC5)CC6)nc(N)c34)nc2n1CC(F)F] |
| 932 | 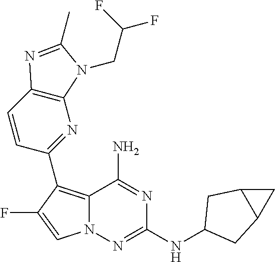 [SMILES: Cc1nc2ccc(-c3c(F)cn4nc(NC5CC6CC6C5)nc(N)c34)nc2n1CC(F)F] |
| 933 | 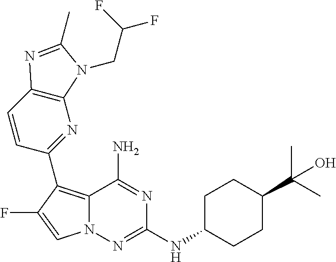 [SMILES: Cc1nc2ccc(-c3c(F)cn4nc(N[C@H]5CC[C@H](C(C)(C)O)CC5)nc(N)c34)nc2n1CC(F)F] |
| 934 | 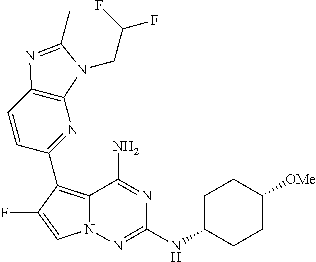 [SMILES: CO[C@H]1CC[C@@H](Nc2nc(N)c3c(-c4ccc5nc(C)n(CC(F)F)c5n4)c(F)cn3n2)CC1] |
| 935 | 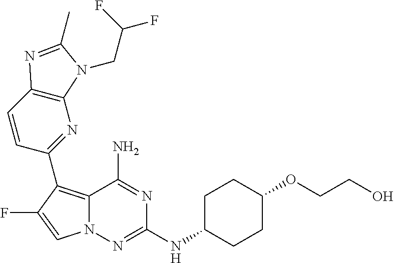 [SMILES: Cc1nc2ccc(-c3c(F)cn4nc(N[C@H]5CC[C@@H](OCCO)CC5)nc(N)c34)nc2n1CC(F)F] |
| 936 | 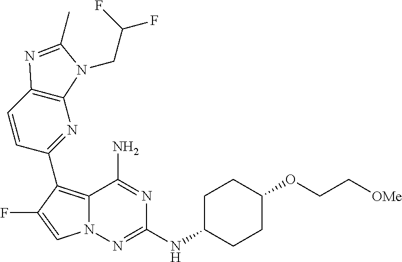 [SMILES: COCCO[C@H]1CC[C@@H](Nc2nc(N)c3c(-c4ccc5nc(C)n(CC(F)F)c5n4)c(F)cn3n2)CC1] |
| 937 | 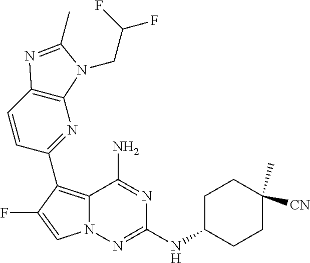 [SMILES: Cc1nc2ccc(-c3c(F)cn4nc(N[C@H]5CC[C@](C)(C#N)CC5)nc(N)c34)nc2n1CC(F)F] |
| 938 | 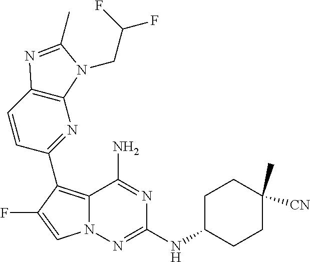 [SMILES: Cc1nc2ccc(-c3c(F)cn4nc(N[C@H]5CC[C@@](C)(C#N)CC5)nc(N)c34)nc2n1CC(F)F] |
| 939 | 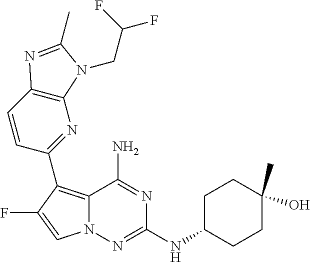 [SMILES: Cc1nc2ccc(-c3c(F)cn4nc(N[C@H]5CC[C@@](C)(O)CC5)nc(N)c34)nc2n1CC(F)F] |
| 940 | 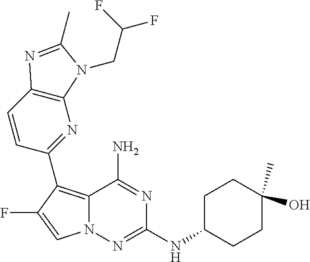 [SMILES: Cc1nc2ccc(-c3c(F)cn4nc(N[C@H]5CC[C@](C)(O)CC5)nc(N)c34)nc2n1CC(F)F] |
| 941 | 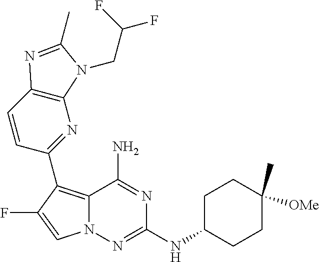 [SMILES: CO[C@]1(C)CC[C@H](Nc2nc(N)c3c(-c4ccc5nc(C)n(CC(F)F)c5n4)c(F)cn3n2)CC1] |
| 942 | 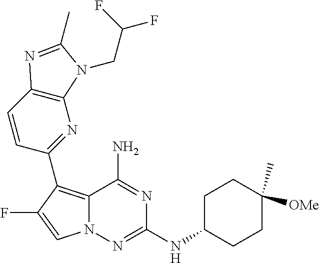 [SMILES: CO[C@]1(C)CC[C@@H](Nc2nc(N)c3c(-c4ccc5nc(C)n(CC(F)F)c5n4)c(F)cn3n2)CC1] |
| 943 | 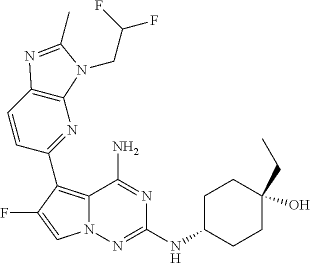 [SMILES: CC[C@]1(O)CC[C@@H](Nc2nc(N)c3c(-c4ccc5nc(C)n(CC(F)F)c5n4)c(F)cn3n2)CC1] |
| 944 | 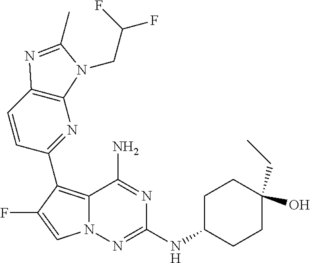 [SMILES: CC[C@]1(O)CC[C@H](Nc2nc(N)c3c(-c4ccc5nc(C)n(CC(F)F)c5n4)c(F)cn3n2)CC1] |
| 945 | 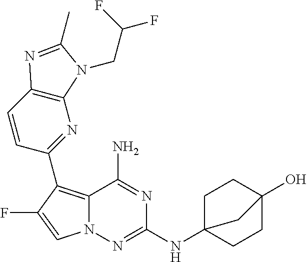 [SMILES: Cc1nc2ccc(-c3c(F)cn4nc(NC56CCC(O)(CC5)C6)nc(N)c34)nc2n1CC(F)F] |
| 946 | 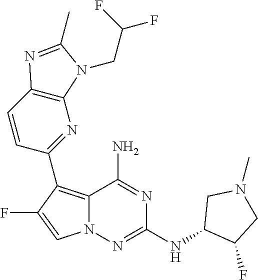 [SMILES: Cc1nc2ccc(-c3c(F)cn4nc(N[C@@H]5CN(C)C[C@@H]5F)nc(N)c34)nc2n1CC(F)F] |
| 947 | 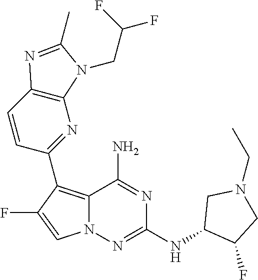 [SMILES: CCN1C[C@H](F)[C@H](Nc2nc(N)c3c(-c4ccc5nc(C)n(CC(F)F)c5n4)c(F)cn3n2)C1] |
| 948 | 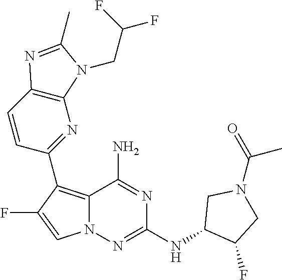 [SMILES: CC(=O)N1C[C@H](F)[C@H](Nc2nc(N)c3c(-c4ccc5nc(C)n(CC(F)F)c5n4)c(F)cn3n2)C1] |
| 949 | 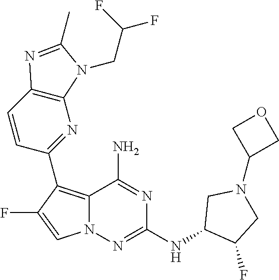 [SMILES: Cc1nc2ccc(-c3c(F)cn4nc(N[C@@H]5CN(C6COC6)C[C@@H]5F)nc(N)c34)nc2n1CC(F)F] |
| 950 | 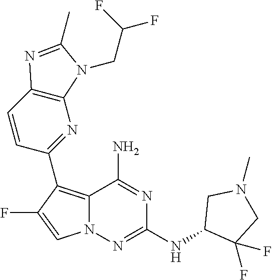 [SMILES: Cc1nc2ccc(-c3c(F)cn4nc(N[C@@H]5CN(C)CC5(F)F)nc(N)c34)nc2n1CC(F)F] |
| 951 | 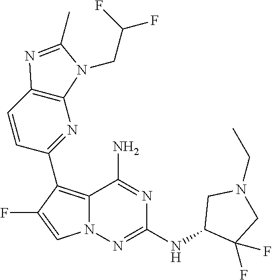 [SMILES: CCN1C[C@@H](Nc2nc(N)c3c(-c4ccc5nc(C)n(CC(F)F)c5n4)c(F)cn3n2)C(F)(F)C1] |
| 952 | 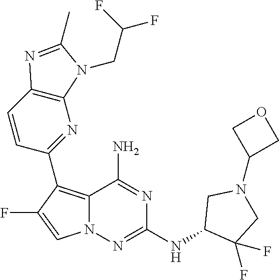 [SMILES: Cc1nc2ccc(-c3c(F)cn4nc(N[C@@H]5CN(C6COC6)CC5(F)F)nc(N)c34)nc2n1CC(F)F] |
| 953 | 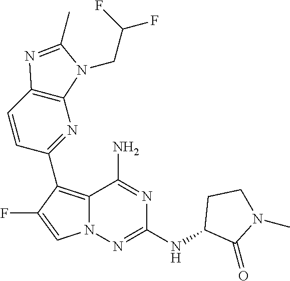 [SMILES: Cc1nc2ccc(-c3c(F)cn4nc(N[C@@H]5CCN(C)C5=O)nc(N)c34)nc2n1CC(F)F] |
| 954 | 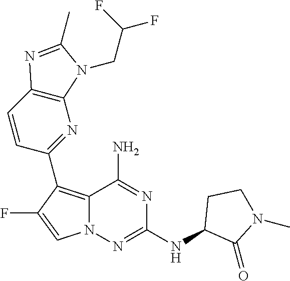 [SMILES: Cc1nc2ccc(-c3c(F)cn4nc(N[C@H]5CCN(C)C5=O)nc(N)c34)nc2n1CC(F)F] |
| 955 | 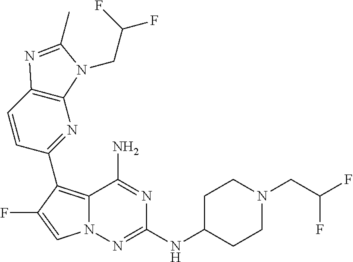 [SMILES: Cc1nc2ccc(-c3c(F)cn4nc(NC5CCN(CC(F)F)CC5)nc(N)c34)nc2n1CC(F)F] |
| 956 | 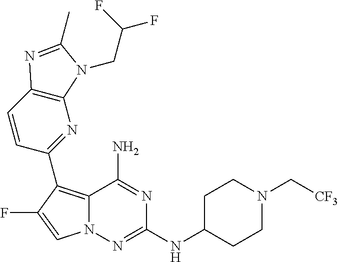 [SMILES: Cc1nc2ccc(-c3c(F)cn4nc(NC5CCN(CC(F)(F)F)CC5)nc(N)c34)nc2n1CC(F)F] |
| 957 | 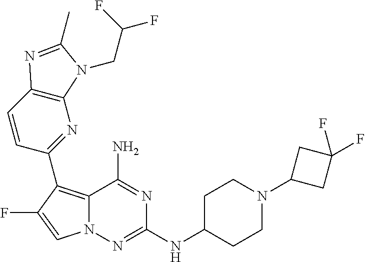 [SMILES: Cc1nc2ccc(-c3c(F)cn4nc(NC5CCN(C6CC(F)(F)C6)CC5)nc(N)c34)nc2n1CC(F)F] |
| 958 | 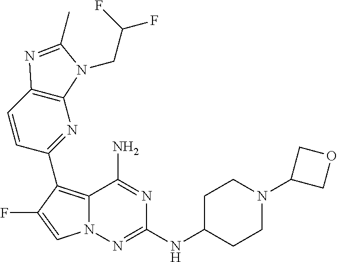 [SMILES: Cc1nc2ccc(-c3c(F)cn4nc(NC5CCN(C6COC6)CC5)nc(N)c34)nc2n1CC(F)F] |
| 959 | 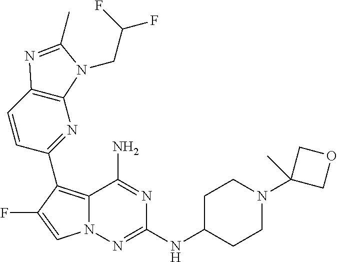 [SMILES: Cc1nc2ccc(-c3c(F)cn4nc(NC5CCN(C6(C)COC6)CC5)nc(N)c34)nc2n1CC(F)F] |
| 960 | 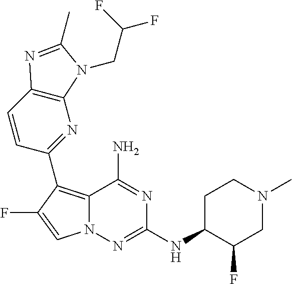 [SMILES: Cc1nc2ccc(-c3c(F)cn4nc(N[C@H]5CCN(C)C[C@H]5F)nc(N)c34)nc2n1CC(F)F] |
| 961 | 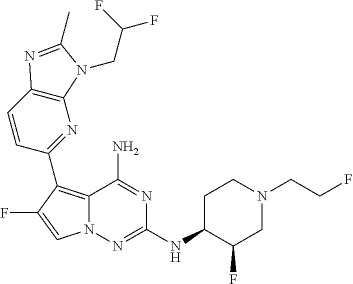 [SMILES: Cc1nc2ccc(-c3c(F)cn4nc(N[C@H]5CCN(CCF)C[C@H]5F)nc(N)c34)nc2n1CC(F)F] |
| 962 | 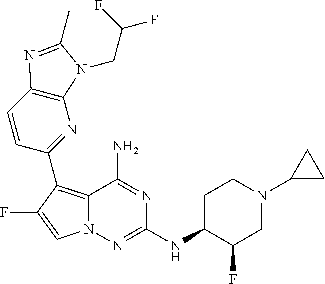 [SMILES: Cc1nc2ccc(-c3c(F)cn4nc(N[C@H]5CCN(C6CC6)C[C@H]5F)nc(N)c34)nc2n1CC(F)F] |
| 963 | 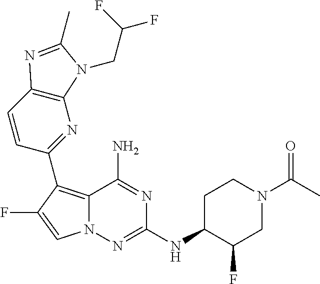 [SMILES: CC(=O)N1CC[C@H](Nc2nc(N)c3c(-c4ccc5nc(C)n(CC(F)F)c5n4)c(F)cn3n2)[C@H](F)C1] |
| 964 | 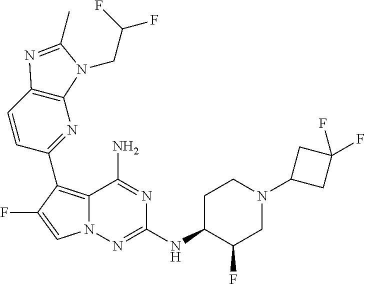 [SMILES: Cc1nc2ccc(-c3c(F)cn4nc(N[C@H]5CCN(C6CC(F)(F)C6)C[C@H]5F)nc(N)c34)nc2n1CC(F)F] |
| 965 | 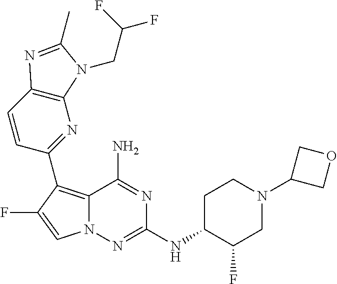 [SMILES: Cc1nc2ccc(-c3c(F)cn4nc(N[C@@H]5CCN(C6COC6)C[C@@H]5F)nc(N)c34)nc2n1CC(F)F] |
| 966 | 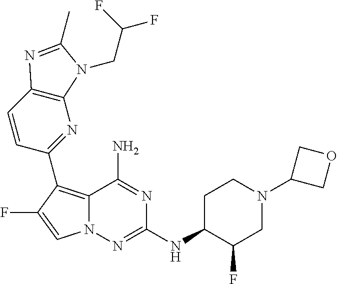 [SMILES: Cc1nc2ccc(-c3c(F)cn4nc(N[C@H]5CCN(C6COC6)C[C@H]5F)nc(N)c34)nc2n1CC(F)F] |
| 967 | 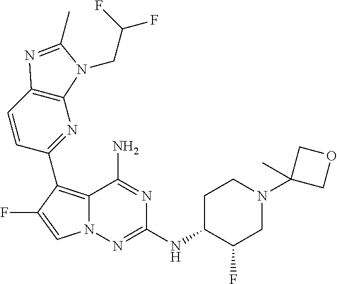 [SMILES: Cc1nc2ccc(-c3c(F)cn4nc(N[C@@H]5CCN(C6(C)COC6)C[C@@H]5F)nc(N)c34)nc2n1CC(F)F] |
| 968 | 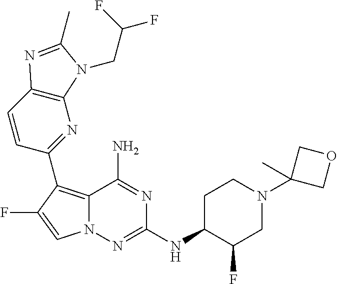 [SMILES: Cc1nc2ccc(-c3c(F)cn4nc(N[C@H]5CCN(C6(C)COC6)C[C@H]5F)nc(N)c34)nc2n1CC(F)F] |
| 969 | 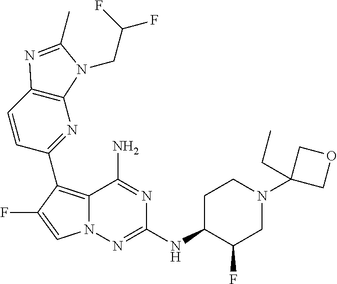 [SMILES: CCC1(N2CC[C@H](Nc3nc(N)c4c(-c5ccc6nc(C)n(CC(F)F)c6n5)c(F)cn4n3)[C@H](F)C2)COC1] |
| 970 | 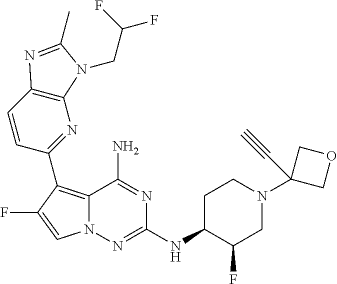 [SMILES: C#CC1(N2CC[C@H](Nc3nc(N)c4c(-c5ccc6nc(C)n(CC(F)F)c6n5)c(F)cn4n3)[C@H](F)C2)COC1] |
| 971 | 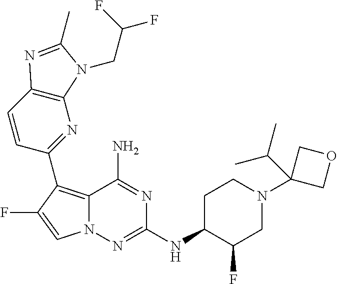 [SMILES: Cc1nc2ccc(-c3c(F)cn4nc(N[C@H]5CCN(C6(C(C)C)COC6)C[C@H]5F)nc(N)c34)nc2n1CC(F)F] |
| 972 | 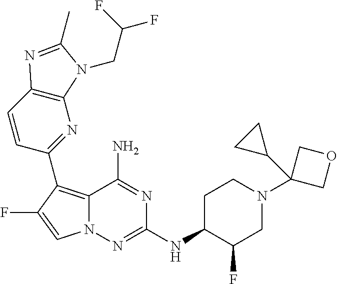 [SMILES: Cc1nc2ccc(-c3c(F)cn4nc(N[C@H]5CCN(C6(C7CC7)COC6)C[C@H]5F)nc(N)c34)nc2n1CC(F)F] |
| 973 | 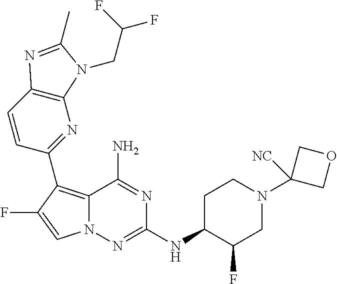 [SMILES: Cc1nc2ccc(-c3c(F)cn4nc(N[C@H]5CCN(C6(C#N)COC6)C[C@H]5F)nc(N)c34)nc2n1CC(F)F] |
| 974 | 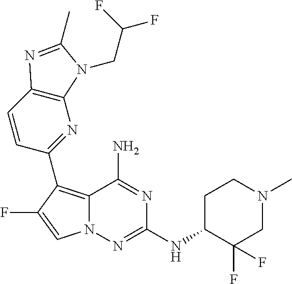 [SMILES: Cc1nc2ccc(-c3c(F)cn4nc(N[C@@H]5CCN(C)CC5(F)F)nc(N)c34)nc2n1CC(F)F] |
| 975 | 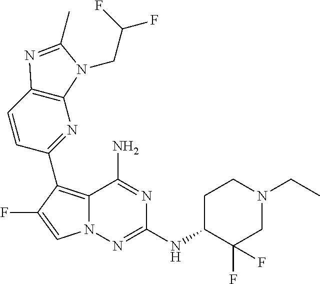 [SMILES: CCN1CC[C@@H](Nc2nc(N)c3c(-c4ccc5nc(C)n(CC(F)F)c5n4)c(F)cn3n2)C(F)(F)C1] |
| 976 | 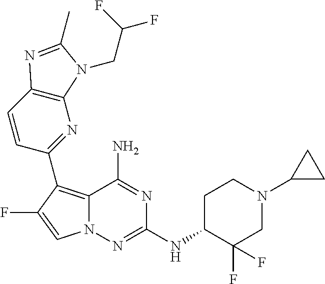 [SMILES: Cc1nc2ccc(-c3c(F)cn4nc(N[C@@H]5CCN(C6CC6)CC5(F)F)nc(N)c34)nc2n1CC(F)F] |
| 977 | 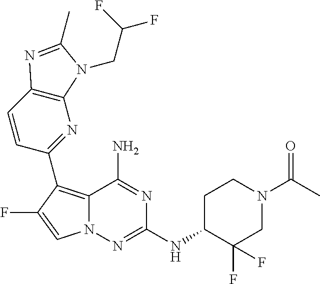 [SMILES: CC(=O)N1CC[C@@H](Nc2nc(N)c3c(-c4ccc5nc(C)n(CC(F)F)c5n4)c(F)cn3n2)C(F)(F)C1] |
| 978 | 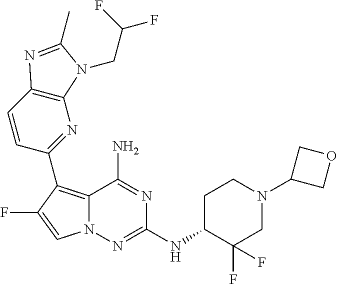 [SMILES: Cc1nc2ccc(-c3c(F)cn4nc(N[C@@H]5CCN(C6COC6)CC5(F)F)nc(N)c34)nc2n1CC(F)F] |
| 979 | 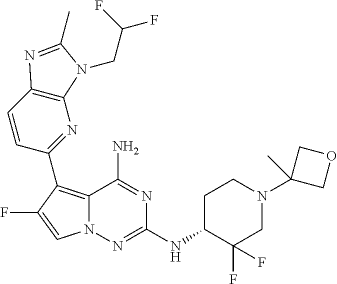 [SMILES: Cc1nc2ccc(-c3c(F)cn4nc(N[C@@H]5CCN(C6(C)COC6)CC5(F)F)nc(N)c34)nc2n1CC(F)F] |
| 980 | 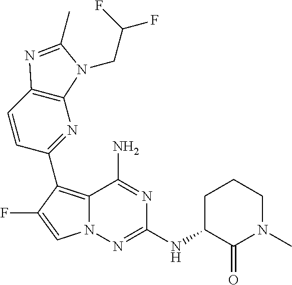 [SMILES: Cc1nc2ccc(-c3c(F)cn4nc(N[C@@H]5CCCN(C)C5=O)nc(N)c34)nc2n1CC(F)F] |
| 981 | 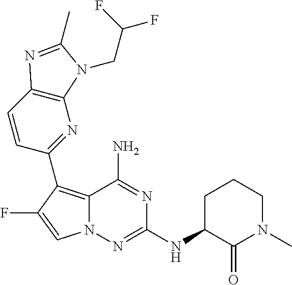 [SMILES: Cc1nc2ccc(-c3c(F)cn4nc(N[C@H]5CCCN(C)C5=O)nc(N)c34)nc2n1CC(F)F] |
| 982 | 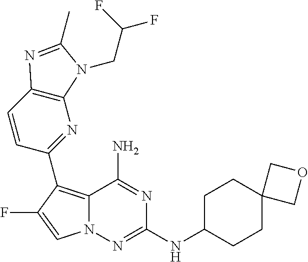 [SMILES: Cc1nc2ccc(-c3c(F)cn4nc(NC5CCC6(CC5)COC6)nc(N)c34)nc2n1CC(F)F] |
| 983 | 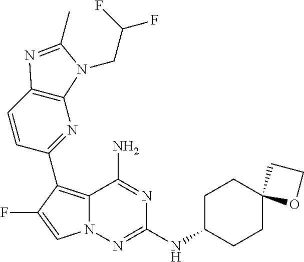 [SMILES: Cc1nc2ccc(-c3c(F)cn4nc(N[C@H]5CC[C@@]6(CCO6)CC5)nc(N)c34)nc2n1CC(F)F] |
| 984 | 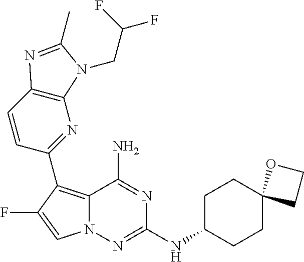 [SMILES: Cc1nc2ccc(-c3c(F)cn4nc(N[C@H]5CC[C@]6(CCO6)CC5)nc(N)c34)nc2n1CC(F)F] |
| 985 | 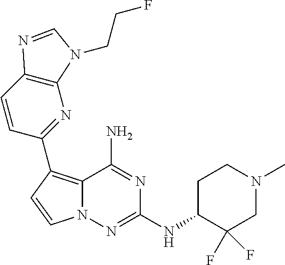 [SMILES: CN1CC[C@@H](Nc2nc(N)c3c(-c4ccc5ncn(CCF)c5n4)ccn3n2)C(F)(F)C1] |
| 986 | 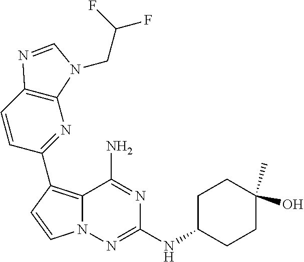 [SMILES: C[C@]1(O)CC[C@H](Nc2nc(N)c3c(-c4ccc5ncn(CC(F)F)c5n4)ccn3n2)CC1] |
| 987 | 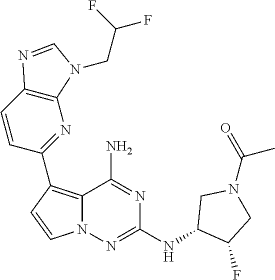 [SMILES: CC(=O)N1C[C@H](F)[C@H](Nc2nc(N)c3c(-c4ccc5ncn(CC(F)F)c5n4)ccn3n2)C1] |
| 988 | 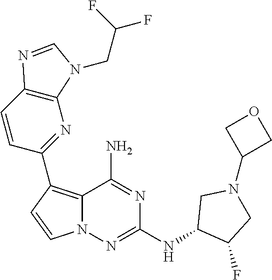 [SMILES: Nc1nc(N[C@@H]2CN(C3COC3)C[C@@H]2F)nn2ccc(-c3ccc4ncn(CC(F)F)c4n3)c12] |
| 989 | 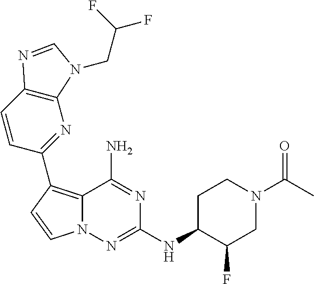 [SMILES: CC(=O)N1CC[C@H](Nc2nc(N)c3c(-c4ccc5ncn(CC(F)F)c5n4)ccn3n2)[C@H](F)C1] |
| 990 | 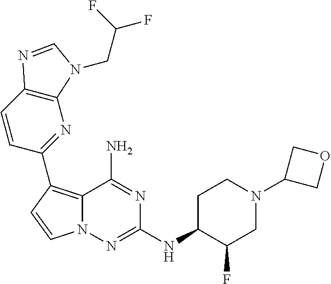 [SMILES: Nc1nc(N[C@H]2CCN(C3COC3)C[C@H]2F)nn2ccc(-c3ccc4ncn(CC(F)F)c4n3)c12] |
| 991 | 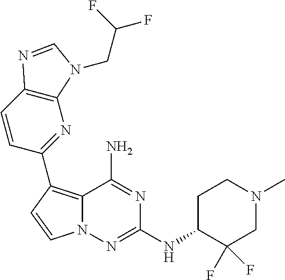 [SMILES: CN1CC[C@@H](Nc2nc(N)c3c(-c4ccc5ncn(CC(F)F)c5n4)ccn3n2)C(F)(F)C1] |
| 992 | 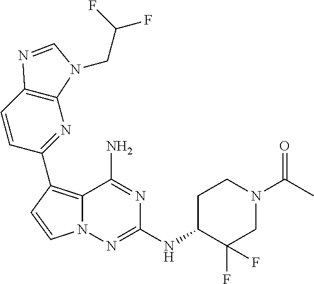 [SMILES: CC(=O)N1CC[C@@H](Nc2nc(N)c3c(-c4ccc5ncn(CC(F)F)c5n4)ccn3n2)C(F)(F)C1] |
| 993 | 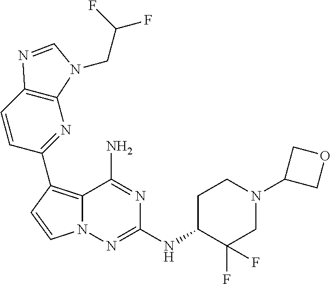 [SMILES: Nc1nc(N[C@@H]2CCN(C3COC3)CC2(F)F)nn2ccc(-c3ccc4ncn(CC(F)F)c4n3)c12] |
| 994 | 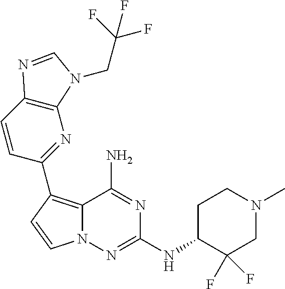 [SMILES: CN1CC[C@@H](Nc2nc(N)c3c(-c4ccc5ncn(CC(F)(F)F)c5n4)ccn3n2)C(F)(F)C1] |
| 995 | 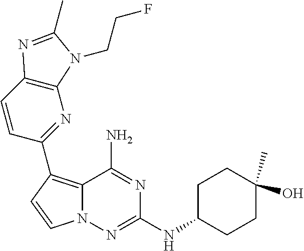 [SMILES: Cc1nc2ccc(-c3ccn4nc(N[C@H]5CC[C@](C)(O)CC5)nc(N)c34)nc2n1CCF] |
| 996 | 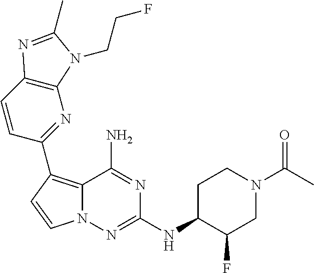 [SMILES: CC(=O)N1CC[C@H](Nc2nc(N)c3c(-c4ccc5nc(C)n(CCF)c5n4)ccn3n2)[C@H](F)C1] |
| 997 | 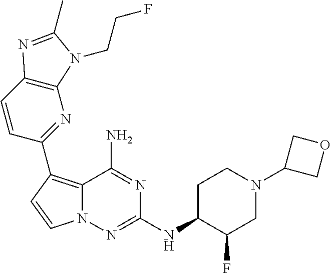 [SMILES: Cc1nc2ccc(-c3ccn4nc(N[C@H]5CCN(C6COC6)C[C@H]5F)nc(N)c34)nc2n1CCF] |
| 998 | 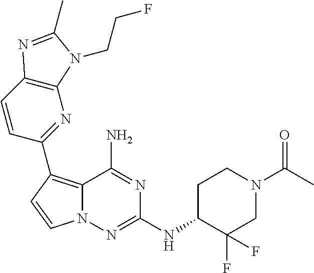 [SMILES: CC(=O)N1CC[C@@H](Nc2nc(N)c3c(-c4ccc5nc(C)n(CCF)c5n4)ccn3n2)C(F)(F)C1] |
| 999 | 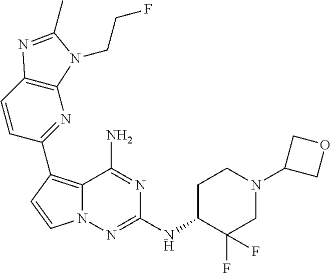 [SMILES: Cc1nc2ccc(-c3ccn4nc(N[C@@H]5CCN(C6COC6)CC5(F)F)nc(N)c34)nc2n1CCF] |
| 1000 | 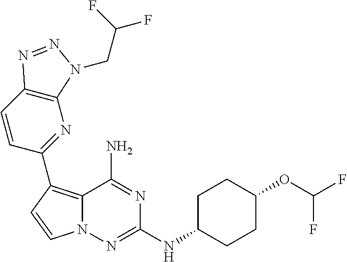 [SMILES: Nc1nc(N[C@H]2CC[C@@H](OC(F)F)CC2)nn2ccc(-c3ccc4nnn(CC(F)F)c4n3)c12] |
| 1001 | 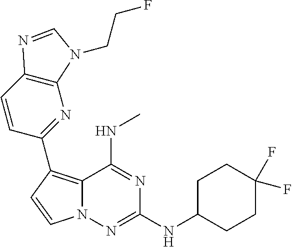 [SMILES: CNc1nc(NC2CCC(F)(F)CC2)nn2ccc(-c3ccc4ncn(CCF)c4n3)c12] |
| 1002 | 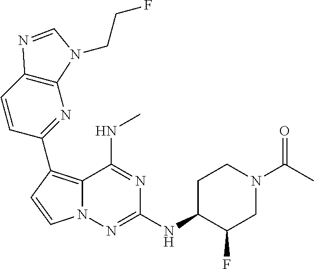 [SMILES: CNc1nc(N[C@H]2CCN(C(C)=O)C[C@H]2F)nn2ccc(-c3ccc4ncn(CCF)c4n3)c12] |
| 1003 | 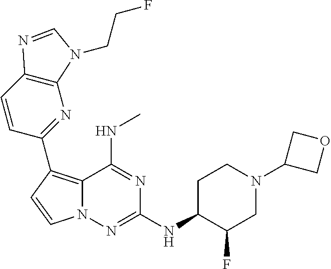 [SMILES: CNc1nc(N[C@H]2CCN(C3COC3)C[C@H]2F)nn2ccc(-c3ccc4ncn(CCF)c4n3)c12] |
| 1004 | 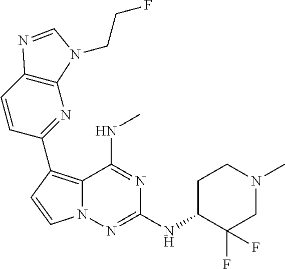 [SMILES: CNc1nc(N[C@@H]2CCN(C)CC2(F)F)nn2ccc(-c3ccc4ncn(CCF)c4n3)c12] |
| 1005 | 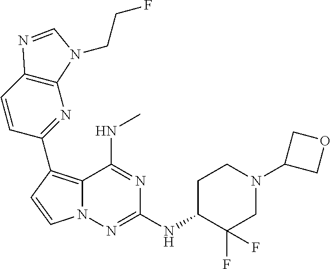 [SMILES: CNc1nc(N[C@@H]2CCN(C3COC3)CC2(F)F)nn2ccc(-c3ccc4ncn(CCF)c4n3)c12] |
| 1006 | 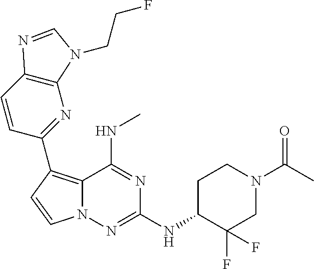 [SMILES: CNc1nc(N[C@@H]2CCN(C(C)=O)CC2(F)F)nn2ccc(-c3ccc4ncn(CCF)c4n3)c12] |
| 1007 | 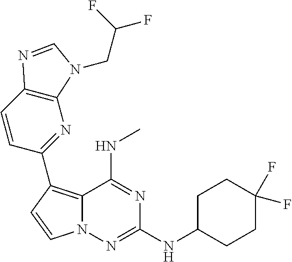 [SMILES: CNc1nc(NC2CCC(F)(F)CC2)nn2ccc(-c3ccc4ncn(CC(F)F)c4n3)c12] |
| 1008 | 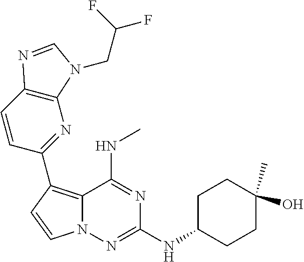 [SMILES: CNc1nc(N[C@H]2CC[C@](C)(O)CC2)nn2ccc(-c3ccc4ncn(CC(F)F)c4n3)c12] |
| 1009 | 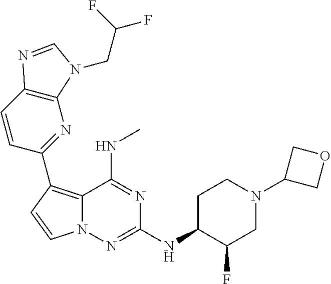 [SMILES: CNc1nc(N[C@H]2CCN(C3COC3)C[C@H]2F)nn2ccc(-c3ccc4ncn(CC(F)F)c4n3)c12] |
| 1010 | 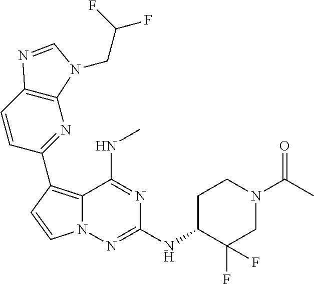 [SMILES: CNc1nc(N[C@@H]2CCN(C(C)=O)CC2(F)F)nn2ccc(-c3ccc4ncn(CC(F)F)c4n3)c12] |
| 1011 | 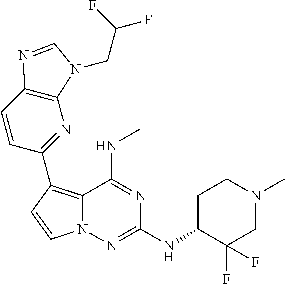 [SMILES: CNc1nc(N[C@@H]2CCN(C)CC2(F)F)nn2ccc(-c3ccc4ncn(CC(F)F)c4n3)c12] |
| 1012 | 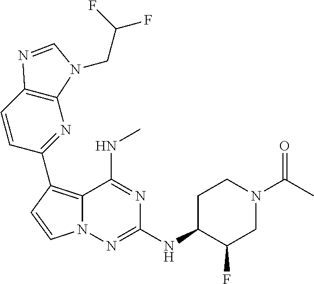 [SMILES: CNc1nc(N[C@H]2CCN(C(C)=O)C[C@H]2F)nn2ccc(-c3ccc4ncn(CC(F)F)c4n3)c12] |
| 1013 | 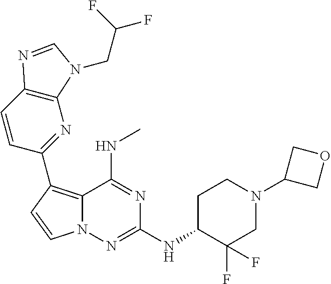 [SMILES: CNc1nc(N[C@@H]2CCN(C3COC3)CC2(F)F)nn2ccc(-c3ccc4ncn(CC(F)F)c4n3)c12] |
| 1014 | 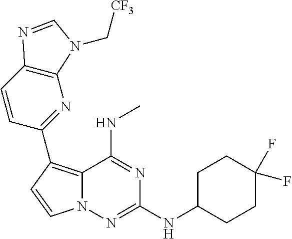 [SMILES: CNc1nc(NC2CCC(F)(F)CC2)nn2ccc(-c3ccc4ncn(CC(F)(F)F)c4n3)c12] |
| 1015 | 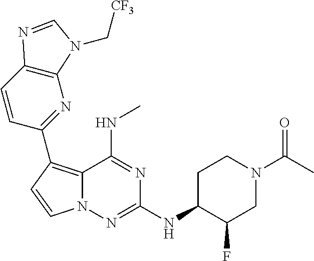 [SMILES: CNc1nc(N[C@H]2CCN(C(C)=O)C[C@H]2F)nn2ccc(-c3ccc4ncn(CC(F)(F)F)c4n3)c12] |
| 1016 | 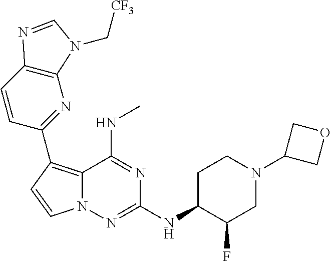 [SMILES: CNc1nc(N[C@H]2CCN(C3COC3)C[C@H]2F)nn2ccc(-c3ccc4ncn(CC(F)(F)F)c4n3)c12] |
| 1017 | 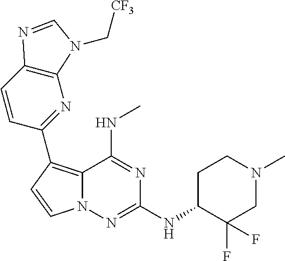 [SMILES: CNc1nc(N[C@@H]2CCN(C)CC2(F)F)nn2ccc(-c3ccc4ncn(CC(F)(F)F)c4n3)c12] |
| 1018 | 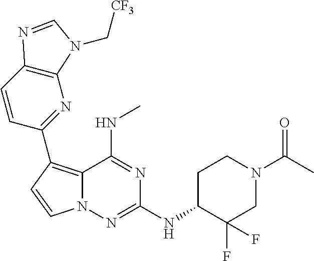 [SMILES: CNc1nc(N[C@@H]2CCN(C(C)=O)CC2(F)F)nn2ccc(-c3ccc4ncn(CC(F)(F)F)c4n3)c12] |
| 1019 | 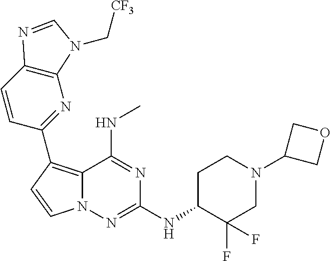 [SMILES: CNc1nc(N[C@@H]2CCN(C3COC3)CC2(F)F)nn2ccc(-c3ccc4ncn(CC(F)(F)F)c4n3)c12] |
| 1020 | 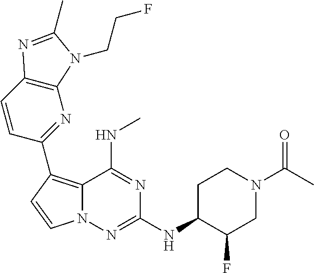 [SMILES: CNc1nc(N[C@H]2CCN(C(C)=O)C[C@H]2F)nn2ccc(-c3ccc4nc(C)n(CCF)c4n3)c12] |
| 1021 | 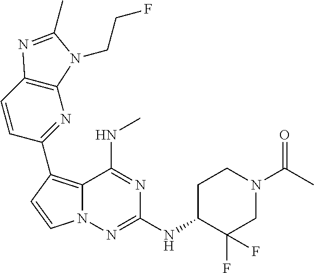 [SMILES: CNc1nc(N[C@@H]2CCN(C(C)=O)CC2(F)F)nn2ccc(-c3ccc4nc(C)n(CCF)c4n3)c12] |
| 1022 | 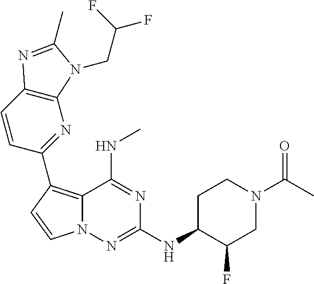 [SMILES: CNc1nc(N[C@H]2CCN(C(C)=O)C[C@H]2F)nn2ccc(-c3ccc4nc(C)n(CC(F)F)c4n3)c12] |
| 1023 | 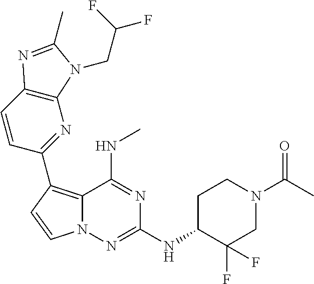 [SMILES: CNc1nc(N[C@@H]2CCN(C(C)=O)CC2(F)F)nn2ccc(-c3ccc4nc(C)n(CC(F)F)c4n3)c12] |
| 1024 | 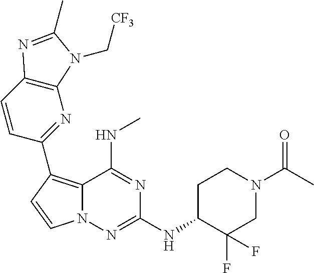 [SMILES: CNc1nc(N[C@@H]2CCN(C(C)=O)CC2(F)F)nn2ccc(-c3ccc4nc(C)n(CC(F)(F)F)c4n3)c12] |
| 1025 | 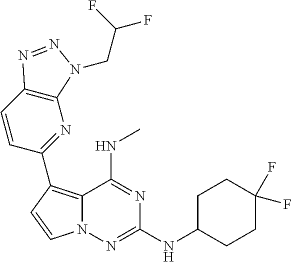 [SMILES: CNc1nc(NC2CCC(F)(F)CC2)nn2ccc(-c3ccc4nnn(CC(F)F)c4n3)c12] |
| 1026 | 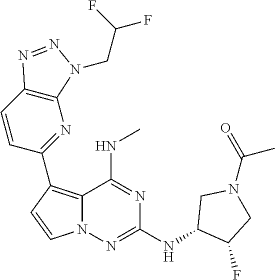 [SMILES: CNc1nc(N[C@@H]2CN(C(C)=O)C[C@@H]2F)nn2ccc(-c3ccc4nnn(CC(F)F)c4n3)c12] |
| 1027 | 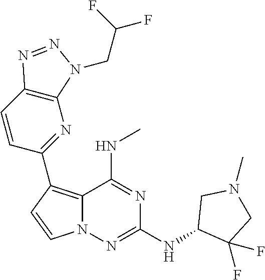 [SMILES: CNc1nc(N[C@@H]2CN(C)CC2(F)F)nn2ccc(-c3ccc4nnn(CC(F)F)c4n3)c12] |
| 1028 | 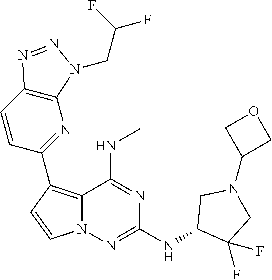 [SMILES: CNc1nc(N[C@@H]2CN(C3COC3)CC2(F)F)nn2ccc(-c3ccc4nnn(CC(F)F)c4n3)c12] |
| 1029 | 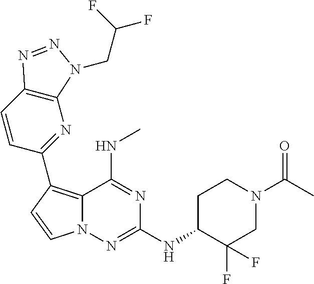 [SMILES: CNc1nc(N[C@@H]2CCN(C(C)=O)CC2(F)F)nn2ccc(-c3ccc4nnn(CC(F)F)c4n3)c12] |
| 1030 | 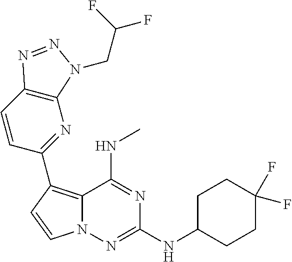 [SMILES: CNc1nc(NC2CCC(F)(F)CC2)nn2ccc(-c3ccc4nnn(CC(F)F)c4n3)c12] |
| 1031 | 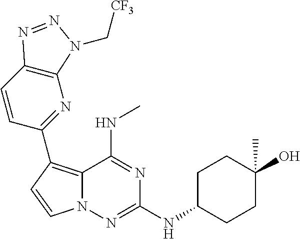 [SMILES: CNc1nc(N[C@H]2CC[C@](C)(O)CC2)nn2ccc(-c3ccc4nnn(CC(F)(F)F)c4n3)c12] |
| 1032 | 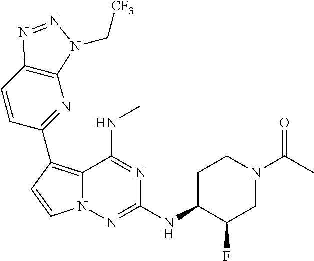 [SMILES: CNc1nc(N[C@H]2CCN(C(C)=O)C[C@H]2F)nn2ccc(-c3ccc4nnn(CC(F)(F)F)c4n3)c12] |
| 1033 | 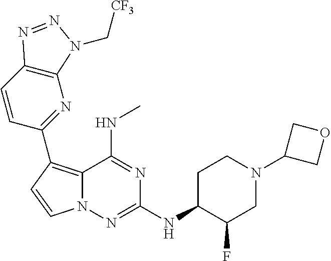 [SMILES: CNc1nc(N[C@H]2CCN(C3COC3)C[C@H]2F)nn2ccc(-c3ccc4nnn(CC(F)(F)F)c4n3)c12] |
| 1034 | 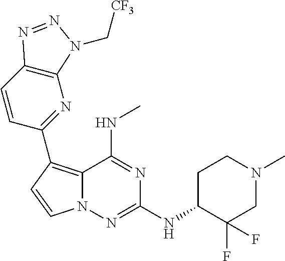 [SMILES: CNc1nc(N[C@@H]2CCN(C)CC2(F)F)nn2ccc(-c3ccc4nnn(CC(F)(F)F)c4n3)c12] |
| 1035 | 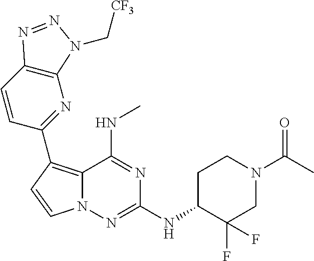 [SMILES: CNc1nc(N[C@@H]2CCN(C(C)=O)CC2(F)F)nn2ccc(-c3ccc4nnn(CC(F)(F)F)c4n3)c12] |
| 1036 | 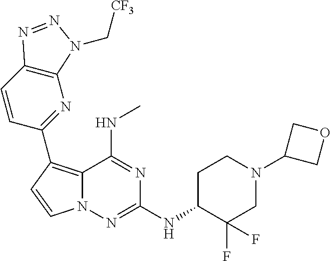 [SMILES: CNc1nc(N[C@@H]2CCN(C3COC3)CC2(F)F)nn2ccc(-c3ccc4nnn(CC(F)(F)F)c4n3)c12] |

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

The compounds provided herein may also be useful in combination (administered together or sequentially) with other known agents.

Non-limiting examples of diseases which can be treated with a combination of a compound of Formula (I) and another active agent are colorectal cancer, ovarian cancer, hepatocellular carcinoma, head and neck squamous cell carcinoma, acute lymphoblastic leukemia (ALL), pancreatic cancer, brain tumors, acute megakaryoblastic leukemia (AMKL), and osteoarthritis. For example, a compound of Formula (I) can be combined with one or more chemotherapeutic compounds.

In some embodiments, hepatocellular carcinoma can be treated with a combination of a compound of Formula (I) and one or more of the following drugs/therapies: sorafenib (Nexavar®); regorafenib (Stivarga®, Regonix®), nivolumab (Opdivo®); lenvatinib (Lenvima®); Pembrolizumab (Keytruda®); cabozantinib (Cometriq®, Cabometyx®); 5-fluorouracil (5-FU®); ramucirumab (Cyramza®); combination of gemcitabine and oxaliplatin (GEMOX). Other therapies that can be performed in combination with a compound of Formula (I) are i) transcatheter arterial chemoembolization (TACE) in combination with doxorubicin (DOXIL®), cisplatin, or mitomycin C (Mitosol®, Mutamycin®, Jelmyto®); ii) low-dose brachytherapy.

In some embodiments, head and neck squamous cell carcinoma can be treated with a combination of a compound of Formula (I) and one or more of the following drugs/therapies: TransOral Robotic Surgery (TORS); TORS with radiation therapy; larotrectinib (Vitrakvi®); EGFR inhibitors, e.g., erlotinib (Tarceva®), osimertinib (Tagrisso®), neratinib (Nerlynx®), gefitinib (Iressa®), cetuximab (Erbitux®), panitumumab (Vectibix®), dacomitinib (Vizimpro®), lapatinib (Tykerb®), necitumumab (Portrazza), and vandetanib (Caprelsa®).

In some embodiments, acute lymphoblastic leukemia (ALL) can be treated with a combination of a compound of Formula (I) and one or more of the following drugs/therapies: remission induction therapy; consolidation therapy; nelarabine (Arranon®); Asparaginase Erwinia Chrysanthemi (Erwinaze®); Asparaginase Erwinia Chrysanthemi (Recombinant)-rywn (Rylaze®); calaspargase Pegol-mknl (Asparlas®); inotuzumab ozogamicin (Besponsa®); blinatumomab (Blincyto®); daunorubicin hydrochloride (Cerubidine®); clofarabine (Clolar®); cyclophosphamide; methotrexate sodium (Trexall®); cytarabine (Cytosar-U®); dasatinib (Sprycel®); dexamethasone; imatinib mesylate (Gleevec®); ponatinib hydrochloride (Iclusig®); mercaptopurine (Purinethol®, Purixan®); tisagenlecleucel (Kymriah®); vincristine sulfate liposome (Marqibo®); pegaspargase (Oncaspar®); prednisone; daunorubicin hydrochloride (Rubidomycin®); and vincristine sulfate.

In some embodiments, pancreatic cancer can be treated with a combination of a compound of Formula (I) and one or more of the following drugs/therapies: ablation and embolization treatment; gemcitabine (Gemzar®); 5-fluorouracil (5-FU®); oxaliplatin (Eloxatin®); albumin-bound paclitaxel (Abraxane®); capecitabine (Xeloda®); cisplatin; irinotecan (Camptosar®); liposomal Irinotecan (Onivyde®); paclitaxel (Taxol®), and docetaxel (Taxotere®).

In some embodiments, brain tumors can be treated with a combination of a compound of Formula (I) and one or more of the following drugs/therapies: carmustine can be administered by way of a gliadel wafer; for glioblastoma and high-grade glioma, radiation therapy with daily low-dose temozolomide (Temodar®) followed by monthly doses of temozolomide after radiation therapy for 6 months to 1 year; lomustine (Gleostine®), procarbazine (Matulane®), and vincristine (Vincasar®), have been used along with radiation therapy; anti-angiogenesis therapy with bevacizumab (Avastin®, Mvasi®); and targeted therapy using larotrectinib (Vitrakvi®).

In some embodiments, acute megakaryoblastic leukemia (AMKL) can be treated with a combination of a compound of Formula (I) and one or more of the following drugs/therapies: cytarabine (Cytosar-U®), etoposide (Vepesid®), and anthracycline drugs. Anthracyclines include daunorubicin (Cerubidine®), idarubicin (Idamycin®), and mitoxantrone (Novantrone®).

In some embodiments, acute myeloid leukemia (AML) can be treated with a combination of a compound of Formula (I) and one or more of the following drugs/therapies: venetoclax and hypomethylating agents (e.g., decitabine, azacitidine), induction chemotherapy (cytarabine and an anthracycline (e.g., daunorubicin or idarubicin), all-trans-retinoic acid (ATRA) and either arsenic trioxide (ATO) monotherapy or an anthracycline), consolidation therapy (cytarabine).

In some embodiments, myelodysplastic syndrome (MDS) can be treated with a combination of a compound of Formula (I) and one or more of the following drugs/therapies: 5-azacytidine, decitabine, lenalidomide, and decitabine/cedazuridine (Ingovi®).

In some embodiments, colorectal cancer can be treated with a combination of a compound of Formula (I) and one or more of the following drugs: 5-Fluorouracil (5-FU), which can be administered with the vitamin-like drug leucovorin (also called folinic acid); capecitabine (XELODA®), irinotecan (CAMPOSTAR®), oxaliplatin (ELOXATIN®). Examples of combinations of these drugs which could be further combined with a compound of Formula (I) are FOLFOX (5-FU, leucovorin, and oxaliplatin), FOLFIRI (5-FU, leucovorin, and irinotecan), FOLFOXIRI (leucovorin, 5-FU, oxaliplatin, and irinotecan) and CapeOx (Capecitabine and oxaliplatin). For rectal cancer, chemo with 5-FU or capecitabine combined with radiation may be given before surgery (neoadjuvant treatment).

In some embodiments, ovarian cancer can be treated with a combination of a compound of Formula (I) and one or more of the following drugs: Topotecan, Liposomal doxorubicin (DOXIL®), Gemcitabine (GEMZAR®), Cyclophosphamide (CYTOXAN®), Vinorelbine (NAVELBINE®), Ifosfamide (IFEX®), Etoposide (VP-16), Altretamine (HEXALEN®), Capecitabine (XELODA®), Irinotecan (CPT-11, CAMPTOSAR®), Melphalan, Pemetrexed (ALIMTA®) and Albumin bound paclitaxel (nab-paclitaxel, ABRAXANE®). Examples of combinations of these drugs which could be further combined with a compound of Formula (I) are TIP (paclitaxel [Taxol], ifosfamide, and cisplatin), VeIP (vinblastine, ifosfamide, and cisplatin) and VIP (etoposide [VP-16], ifosfamide, and cisplatin). Ovarian cancer can also be treated with a combination of a compound of Formula (I) and immune checkpoint blockade (ICB) therapy.

In some embodiments, a compound of Formula (I) can be used to treat cancer in combination with any of the following methods: (a) Hormone therapy such as aromatase inhibitors, LHRH [luteinizing hormone-releasing hormone] analogs and inhibitors, and others; (b) Ablation or embolization procedures such as radiofrequency ablation (RFA), ethanol (alcohol) ablation, microwave thermotherapy and cryosurgery (cryotherapy); (c) Chemotherapy using alkylating agents such as cisplatin and carboplatin, oxaliplatin, mechlorethamine, cyclophosphamide, chlorambucil and ifosfamide; (d) Chemotherapy using anti-metabolites such as azathioprine and mercaptopurine; (e) Chemotherapy using plant alkaloids and terpenoids such as vinca alkaloids (i.e. Vincristine, Vinblastine, Vinorelbine and Vindesine) and taxanes; (f) Chemotherapy using podophyllotoxin, etoposide, teniposide and docetaxel; (g) Chemotherapy using topoisomerase inhibitors such as irinotecan, topotecan, amsacrine, etoposide, etoposide phosphate, and teniposide; (h) Chemotherapy using cytotoxic antibiotics such as actinomycin, anthracyclines, doxorubicin, daunorubicin, valrubicin, idarubicin, epirubicin, bleomycin, plicamycin and mitomycin; (i) Chemotherapy using tyrosine-kinase inhibitors such as Imatinib mesylate (GLEEVEC®, also known as STI-571), Gefitinib (Iressa, also known as ZD1839), Erlotinib (marketed as TARCEVA®), Bortezomib (VELCADE®), tamoxifen, tofacitinib, crizotinib, Bcl-2 inhibitors (e.g. obatoclax, navitoclax (ABT-263), oblimersen (G3139), venetoclax (ABT-199), Gossypol), PARP inhibitors (e.g. Iniparib, Olaparib, Rucaparib, Niraparib, Talazoparib), PI3K inhibitors (e.g. perifosine in a phase III trial), VEGF Receptor 2 inhibitors (e.g. Apatinib), AN-152, (AEZS-108), Braf inhibitors (e.g. vemurafenib, dabrafenib and LGX818), MEK inhibitors (e.g. trametinib and MEK162), CDK inhibitors, (e.g. PD-0332991), salinomycin and Sorafenib; (j) Chemotherapy using monoclonal antibodies such as Rituximab (marketed as MABTHERA® or RITUXAN®), Trastuzumab (Herceptin also known as ErbB2), Cetuximab (marketed as ERBITUX®), and Bevacizumab (marketed as AVASTIN®); (k) Chemotherapy using KRAS G12C inhibitors such as sotorasib (Lumakras® and Lumykras®), adagrasib (MRTX849), and ARS-3248 (Wellspring Biosciences); (l) Chemotherapy using checkpoint inhibitor therapy such as Ipilimumab (Yervoy®), Nivolumab (Opdivo®), Pembrolizumab (Keytruda®), Atezolizumab (Tecentriq®), Avelumab (Bavencio), Durvalumab (Imfinzi), Cemiplimab (Libtayo®), and Spartalizumab (PDR001); (m) Chemotherapy using antibody-drug conjugates (ADC) such as Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, and Loncastuximab tesirine; (n) Chemotherapy using proteasome inhibitors such as carfilzomib, lactacystin, disulfiram, salinosporamide A (marizomib), oprozomib, delanzomib, epoxomicin, MG132, β-hydroxy β-methylbutyric acid (HMB), bortezomib, ixazomib (alone or in in combination with lenalidomide and dexamethasone); and (o) radiation therapy.

In some embodiments, a compound of Formula I, can be used to treat diabetes mellitus in combination with any of the following methods: (a) injections of insulin; (b) biguanides such as metformin (Glucophage), phenformin (DBI), and buformin; (c) thiazolidinediones (TZDs) such as rosiglitazone (Avandia), pioglitazone (Actos), and yroglitazone (Rezulin); (d) lyn kinase activators such as glimepiride (Amaryl®) and tolimidone (MLR-1023); (e) secretagogues such as sulfonylureas (non-limiting examples are acetohexamide, carbutamide, chlorpropamide, glycyclamide (tolcyclamide), metahexamide, tolazamide, tolbutamide, glibenclamide (glyburide), glibornuride, gliclazide, glipizide, gliquidone, glisoxepide, glyclopyramide, and glimepiride) and meglitinides (nonlimiting examples are repaglinide (Prandin), nateglinide (Starlix), and mitiglinide (Glufast)); (f) alpha-glucosidase inhibitors such as acarbose (Glucobay, Precose, Prandase), miglitol (Glyset), and voglibose; (g) injectable incretin mimetics such as glucagon-like peptide-1 (GLP-1) and gastric inhibitory peptide (glucose-dependent insulinotropic peptide, GIP), nonlimiting examples of injectable glucagon-like peptide (GLP) analogs and agonists are exenatide (Exendin-4, marketed as Byetta), liraglutide (Victoza, Saxenda), taspoglutide, lixisenatide (Lyxumia), Semaglutide (Ozempic, Rybelsus), dulaglutide (Trulicity), albiglutide (Tanzeum), nonlimiting examples of dipeptidyl peptidase-4 (DPP-4) inhibitors are sitagliptin (Januvia), vildagliptin (Galvus), saxagliptin (Onglyza), linagliptin (Tradjenta), gemigliptin (Zemiglo), anagliptin (Suiny), teneligliptin (Tenelia), alogliptin (Nesina, Vipidia, Kazano, Vipidomet (with metformin), Oseni, Incresync (with pioglitazone)), trelagliptin (Zafatek, Wedica), omarigliptin (MK-3102), evogliptin (Suganon, Evodine), gosogliptin (Saterex), and dutogliptin; (h) injectable amylin analogues such as pramlintide (Symlin); (i) glycosurics (SGLT2 inhibitors) such as canagliflozin (Invokana, Sulisent, Prominad), dapagliflozin (Forxiga, Farxiga, Edistride), empagliflozin (Jardiance, Sciampa-M), ertugliflozin (Steglatro), ipragliflozin (Suglat), luseogliflozin (Lusefi), remogliflozin etabonate (pro-drug of remogliflozin), sergliflozin etabonate (GW869682X), sotagliflozin (Zynquista), and tofogliflozin (CSG452).

In some embodiments, a compound of Formula (I) can be used to treat osteoarthritis in combination with any of the following methods: (d) injections of a Wnt signaling pathway inhibitor (e.g. lorecivivint); (a) Nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g. Hyalgan, Synvisc); (e) narcotics, like codeine; (f) in combination with braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) in combination with a chronic pain class.

In some embodiments, a compound of Formula (I) can be used to treat Alzheimer's disease in combination with aducanumab (Aduhelm™); acetylcholinesterase inhibitors, e.g., tacrine, rivastigmine (Exelon®), galantamine (Razadyne® and GalantaMind™), and donepezil (Aricept®); and memantine (Axura®, Ebixa®, Namenda®).

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release, or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants, and the like, for prolonged and/or timed, pulsed administration at a predetermined rate.

The compounds can be administered either alone or in combination with a conventional pharmaceutical carrier, excipient, or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol, or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable and could be higher if the composition is a solid or suspension, which could be subsequently diluted to the above percentages.

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formula (I) disclosed herein intended for respiratory delivery (either systemic or local) can be administered as aqueous formulations, as non-aqueous solutions, or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formula (I) disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the disclosure can be incorporated into a gel formulation (e.g., U.S. Pat. Nos. 4,474,752 and 6,911,211).

In some embodiments, compounds of Formula (I) disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid compound of Formula (I) can be delivered from the reservoir of an external or internal implanted pumping system.

Formulations of the disclosure also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formula (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration of the drug (either as a solution, colloid, suspension or a complex) can be prepared by mixing a compound provided herein with a suitable non-irritating excipient that is solid at ordinary temperatures but liquid at the rectal temperature and will therefore melt or erode/dissolve in the rectum and release the compound. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter, is first melted.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of glioblastoma, ovarian, breast, pancreatic cancers, acute lymphoblastic leukemia, acute megakaryoblastic leukemia, chronic myeloid leukemia, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, Autism, Dementia, Epilepsy, Huntington's Disease, and Multiple Sclerosis.

### Methods of Treatment

The compounds and compositions provided herein can be used as inhibitors of DYRK1A, and thus can be used to treat a variety of disorders and diseases in which over expression of DYRK1A is implicated, such as cancer and neurological conditions/disorders/diseases. Non-limiting examples of diseases which can be treated with the compounds and compositions provided herein include a variety of cancers, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, tauopathies (e.g., Pick's disease, progressive supranuclear palsy, corticobasal degeneration, argyrophilic grain disease, globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

The gene encoding DYRK1A is located on chromosome 21, within the Down syndrome critical region (DSCR), the triploidy of which is responsible for most Down syndrome-associated deficiencies (FEBS Journal (2011), 278, 246-256). There is considerable genetical and pharmacological evidence showing that the mere 1.5-fold overexpression of DYRK1A is responsible for most cognitive deficits observed in Down syndrome patients (Pharmacology & Therapeutics (2019), 194, 199-221 and Brain Science (2018), 8(10), 187). Genetical normalization of DYRK1A levels or pharmacological inhibition of its catalytic activity restores cognitive functions. The development of pharmacological inhibitors of DYRK1A is a major avenue for the treatment of cognitive deficits associated with Down syndrome.

DYRK1A and DYRK1B are utilized during human cytomegalovirus (HCMV) placental replication. Inhibition of DYRKs prevent replication of various viruses, including hepatitis C virus (HCV), human cytomegalovirus (HCMV), human immunodeficiency virus type 1 (HIV-1), and herpes simplex virus 1 (HSV-1) (Journal of Virology (2020), 94(6) and PLoS ONE (2015), 10, e0144229).

There is a growing body of evidence showing that DYRK1A/1B inhibitors induce the proliferation of insulin-producing pancreatic β-cells, making DYRK1A/1B kinases attractive therapeutic targets for β-cell regeneration for both type 1 and type 2 diabetes mellitus and gestational diabetes (Nature Communications (2015), 6(8372); Diabetes (2016), 65(6), 1660-1671; JCI Insight (2020), 5(1), e132594; Science Translational Medicine (2020), 12(530); International Journal of Molecular Sciences (2021), 22(16), 9083; and Journal of Medicinal Chemistry (2021), 64(6), 2901-2922). Other forms of diabetes that may be treated with DYRK inhibitors are maturity onset diabetes of the young (MODY, monogenic diabetes), cases of diabetes that are caused by the body's tissue receptors not responding to insulin, double diabetes (when a type 1 diabetic becomes insulin resistant), diabetes associated with excessive secretion of insulin-antagonistic hormones, malnutrition-related diabetes mellitus (ICD-10 code E12), and diabetes caused by any genetic mutations (autosomal or mitochondrial) that leads to defects in beta cell function.

There is abundant literature linking DYRK1A with solid cancers and leukemias (Pharmacology & Therapeutics (2015), 151, 87-98; Cancers (2020), 12(8), 2106; and Cellular and Molecular Life Sciences (2021), 78, 603-619). The most prominent examples are pancreatic cancer (Gut (2019), 68(8), 1465-1476 and Gene (2020), 758, 144960), brain tumors, glioblastoma (Journal of Clinical Investigation (2013), 123(6), 2475-2487), acute megakaryoblastic leukemia (AMKL) (Journal of Clinical Investigation (2012), 122(3), 948-962), and acute lymphoblastic leukemia (ALL) (Journal of Clinical Investigation (2021), 131(1), e135937). Other cancers linked to DYRK1A are ovarian (Frontiers in Oncology (2021), 11, 637193), head and neck squamous cell carcinoma (Scientific Reports (2016), 6, 36132), hepatocellular carcinoma (Cell Death & Disease (2021), 12, 125), DYRK1A regulates DNA damage response (Scientific Reports (2019), 9, 6014 and Scientific Reports (2019), 9, 6539). In some situations, DYRK1A appears to function as a tumor-suppressor protein (Molecular & Cellular Oncology (2015), 2(1), e970048 and Nature (2016), 529, 172-177).

Other cancers can also be treated with the compounds and compositions described herein.

More particularly, cancers that may be treated by the compounds, compositions and methods described herein include, but are not limited to, the following:
1) Breast cancers, including, for example ER⁺ breast cancer, ER⁻ breast cancer, her2⁻ breast cancer, her2⁺ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; chemoresistant breast cancers (TNBC), and miscellaneous malignant neoplasms. Further examples of breast cancers can include luminal A, luminal B, basal A, basal B, and triple negative breast cancer, which is estrogen receptor negative (ER⁻), progesterone receptor negative, and her2 negative (her2⁻). In some embodiments, breast cancer may have a high risk Oncotype score.
2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.
3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; chemoresistant small cell lung cancer (SCLC), and mesothelioma.
4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; colon cancers with APC gene mutations; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.
5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.
6) Liver cancers, including, for example, hepatoma, e.g., hepatocellular carcinoma; cholangiocarcinoma; hepatoblastoma; angiosarcoma; hepatocellular adenoma; and hemangioma.
7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.
8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, oligodendrocytoma, schwannoma, retinoblastoma, and congenital tumors; and cancers of the spinal cord, e.g., neurofibroma, meningioma, glioma, and sarcoma.
9) Gynecological cancers, including, for example, cancers of the uterus, e.g., endometrial cancers (e.g., carcinoma, endometrioid adenocarcinoma, serous carcinoma, clear cell carcinoma, mucinous carcinomas, mixed or undifferentiated carcinoma (including mixed Müllerian tumor), endometrial stromal sarcoma, squamous cell carcinoma of the endometrium, urothelial carcinoma, endometrial cancer with CTNNB1 mutations); cancers of the cervix, e.g., cervical carcinoma, and pre tumor cervical dysplasia; cancers of the ovaries, e.g., BRCA-mutant ovarian cancer, surface epithelial-stromal tumors (epithelial ovarian cancer (Type 1 (endometroid, mucinous, clear cell, low grade serous) or Type 2 (poorly differentiated, carcinosarcoma, and high grade serous))), ovarian carcinoma, including serous cystadenocarcinoma, mucinous cystadenocarcinoma, endometrioid tumors, small cell ovarian cancer (small cell ovarian cancer of hypercalcemic type, small cell ovarian cancer of pulmonary type) unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma, primary fallopian tube cancer; Primary peritoneal cancer (also known as serous surface papillary carcinoma, primary peritoneal carcinoma, extra-ovarian serous carcinoma, primary serous papillary carcinoma, and psammomacarcinoma).
10) Hematologic cancers, including, for example, cancers of the blood, e.g., acute myeloid leukemia, chronic myeloid leukemia, myelodysplastic syndromes (refractory cytopenia with unilineage dysplasia (refractory anemia, refractory neutropenia, and refractory thrombocytopenia), refractory anemia with ring sideroblasts, refractory cytopenia with multilineage dysplasia, refractory anemias with excess blasts I and II, refractory cytopenia of childhood), and myeloproliferative neoplasms, acute lymphoblastic leukemia, chronic lymphocytic leukemia, myeloproliferative diseases, multiple myeloma, myelodysplastic syndrome, myelodysplastic-myeloproliferative diseases, Hodgkin's lymphoma, non-Hodgkin's lymphoma (malignant lymphoma) and Waldenstrom's macroglobulinemia.
11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.
12) Adrenal gland cancers, including, for example, neuroblastoma.
13) Soft-tissue sarcomas (STS) such as fibrosarcoma, malignant fibrous histiocytoma, dermatofibrosarcoma, liposarcoma, rhabdomyosarcoma, leiomyosarcoma, hemangiosarcoma, Kaposi's sarcoma, lymphangiosarcoma, synovial sarcoma, malignant peripheral nerve sheath tumors (also called neurofibrosarcomas, malignant schwannomas, and neurogenic sarcomas), neurofibrosarcoma, extraskeletal chondrosarcoma, extraskeletal osteosarcoma, extraskeletal myxoid chondrosarcoma, extraskeletal mesenchymal, embryonal, alveolar soft part sarcoma, and infantile hemangio-pericytoma.

More particularly, tumors of the central nervous system that may be treated by the compounds, compositions and methods described herein include:
1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.
2) Oligodendroglial tumors, e.g., oligodendroglioma and anaplastic oligodendroglioma.
3) Oligoastrocytic tumors, e.g., oligoastrocytoma and anaplastic oligoastrocytoma.
4) Ependymal tumors, e.g., subependymoma, myxopapillary ependymoma, ependymoma, (cellular, papillary, clear cell, tanycytic), and anaplastic (malignant) ependymoma.
5) Choroid plexus tumors, e.g., choroid plexus papilloma, atypical choroid plexus papilloma, and choroid plexus carcinoma.
6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.
7) Pineal tumors, e.g., pineocytoma, pineoblastoma, papillary tumors ofthe pineal region, and pineal parenchymal tumor of intermediate differentiation.
8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.
9) Neuroblastic tumors, e.g., olfactory (esthesioneuroblastoma), olfactory neuroepithelioma, and neuroblastomas of the adrenal gland and sympathetic nervous system.
10) Glial tumors, e.g., astroblastoma, chordoid glioma of the third ventricle, and angiocentric glioma.
11) Tumors of cranial and paraspinal nerves, e.g., schwannoma, neurofibroma Perineurioma, and malignant peripheral nerve sheath tumor.
12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.
13) Tumors of the hematopoietic system, e.g., malignant Lymphomas, plasmocytoma, and granulocytic sarcoma.
14) Germ cell tumors, e.g., germinoma, embryonal carcinoma, yolk sac tumor, choriocarcinoma, teratoma, and mixed germ cell tumors.
15) Tumors of the sellar region, e.g., craniopharyngioma, granular cell tumor, pituicytoma, and spindle cell oncocytoma of the adenohypophysis.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

There is mounting evidence for a role of DYRK1A in the onset of Alzheimer's Disease (Future Medicinal Chemistry (2016), 8(6), 681-696 and European Journal of Medicinal Chemistry (2018), 158, 559-592). DYRK1A phosphorylates key substrates involved in Alzheimer's Disease and dementia: Tau, septin 4, amyloid precursor protein (APP), presenilin 1, neprilysin, Munc18-1, α-synuclein, RCAN1, and β-tubulin. By modulating alternative splicing of Tau exon 10, DYRK1A favors the production of the 3R-Tau splice isoform (characteristic for DS/AD/tauopathy) over the 4R-Tau isoform (Journal of Biological Chemistry (2015), 290, 15219-15237).

Genome-wide association studies (GWAS) have revealed that DYRK1A is a risk factor for Parkinson's Disease (The Lancet Neurology (2019), 18(12), 1091-1102). DYRK1A phosphorylates key factors for Parkinson's Disease such as parkin, septin 4, and α-synuclein. Upregulation of micro-RNAs specific for Parkinson's Disease targets DYRK1A expression. There is further evidence that DYRK1A expression is increased in Parkinson's Disease and in Pick's disease (Neurobiology of Disease (2005), 20(2), 392-400).

The compounds and compositions provided herein can be used as inhibitors and/or modulators of the enzyme DYRK1A, and thus can be used to treat a variety of disorders and diseases associated with tau protein, including, but not limited to, Alzheimer's disease, amyotrophic lateral sclerosis (ALS), down syndrome, frontotemporal dementia (FTD) including FTD with Parkinsonism-17 (FTDP-17), behavioural variant frontotemporal dementia (bvFTD), FTD in patients with motor neuron disease (MND) (typically amyotrophic lateral sclerosis, also called FTD-ALS), corticobasal degeneration (CBD) (also called corticobasal ganglionic degeneration), progressive supranuclear palsy, primary progressive aphasia (PPA), globular glial tauopathy (GGT), myotonic dystrophy type 1 (DM1) (also called Steinert disease), myotonic dystrophy type 2 (DM2) (also called proximal myotonic myopathy), Guam complex, argyrophilic grain disease, dementia pugilistica, post-encephalitic parkinsonism, Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

Non-limiting examples of neurological disorders (e.g., neurological conditions and neurological diseases) which can be treated with the compounds and compositions provided herein include Alzheimer's disease, aphasia, apraxia, arachnoiditis, ataxia telangiectasia, attention deficit hyperactivity disorder, auditory processing disorder, autism, alcoholism, Bell's palsy, bipolar disorder, brachial plexus injury, Canavan disease, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, cerebral vasculitis, cervical spinal stenosis, Charcot-Marie-Tooth disease, Chiari malformation, chronic fatigue syndrome, chronic inflammatory demyelinating polyneuropathy (CIDP), chronic pain, Coffin-Lowry syndrome, complex regional pain syndrome, compression neuropathy, congenital facial diplegia, corticobasal degeneration, cranial arteritis, craniosynostosis, Creutzfeldt-Jakob disease, cumulative trauma disorder, Cushing's syndrome, cytomegalic inclusion body disease (CIBD), Dandy-Walker syndrome, Dawson disease, De Morsier's syndrome, Dejerine-Klumpke palsy, Dejerine-Sottas disease, delayed sleep phase syndrome, dementia, dermatomyositis, developmental dyspraxia, diabetic neuropathy, diffuse sclerosis, Dravet syndrome, dysautonomia, dyscalculia, dysgraphia, dyslexia, dystonia, empty sella syndrome, encephalitis, encephalocele, encephalotrigeminal angiomatosis, encopresis, epilepsy, Erb's palsy, erythromelalgia, essential tremor, Fabry's disease, Fahr's syndrome, familial spastic paralysis, febrile seizure, Fisher syndrome, Friedreich's ataxia, fibromyalgia, Foville's syndrome, Gaucher's disease, Gerstmann's syndrome, giant cell arteritis, giant cell inclusion disease, globoid cell leukodystrophy, gray matter heterotopia, Guillain-Barré syndrome, HTLV-1 associated myelopathy, Hallervorden-Spatz disease, hemifacial spasm, hereditary spastic paraplegia, heredopathia atactica polyneuritiformis, herpes zoster oticus, herpes zoster, Hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile Refsum disease, infantile spasms, inflammatory myopathy, intracranial cyst, intracranial hypertension, Joubert syndrome, Karak syndrome, Kearns-Sayre syndrome, Kennedy disease, Kinsbourne syndrome, Klippel Feil syndrome, Krabbe disease, Kugelberg-Welander disease, kuru, Lafora disease, Lambert-Eaton myasthenic syndrome, Landau-Kleffner syndrome, lateral medullary (Wallenberg) syndrome, Leigh's disease, Lennox-Gastaut syndrome, Lesch-Nyhan syndrome, leukodystrophy, Lewy body dementia, lissencephaly, locked-in syndrome, Lou Gehrig's disease, lumbar disc disease, lumbar spinal stenosis, Lyme disease, Machado-Joseph disease (Spinocerebellar ataxia type 3), macrencephaly, macropsia, megalencephaly, Melkersson-Rosenthal syndrome, Meniere's disease, meningitis, Menkes disease, metachromatic leukodystrophy, microcephaly, micropsia, Miller Fisher syndrome, misophonia, mitochondrial myopathy, Mobius syndrome, monomelic amyotrophy, motor neuron disease, motor skills disorder, Moyamoya disease, mucopolysaccharidoses, multi-infarct dementia, multifocal motor neuropathy, multiple sclerosis, multiple system atrophy, muscular dystrophy, myalgic encephalomyelitis, myasthenia gravis, myelinoclastic diffuse sclerosis, myoclonic Encephalopathy of infants, myoclonus, myopathy, myotubular myopathy, myotonia congenital, narcolepsy, neurofibromatosis, neuroleptic malignant syndrome, lupus erythematosus, neuromyotonia, neuronal ceroid lipofuscinosis, Niemann-Pick disease, O'Sullivan-McLeod syndrome, occipital Neuralgia, occult Spinal Dysraphism Sequence, Ohtahara syndrome, olivopontocerebellar atrophy, opsoclonus myoclonus syndrome, optic neuritis, orthostatic hypotension, palinopsia, paresthesia, Parkinson's disease, paramyotonia Congenita, paraneoplastic diseases, paroxysmal attacks, Parry-Romberg syndrome, Pelizaeus-Merzbacher disease, periodic paralyses, peripheral neuropathy, photic sneeze reflex, phytanic acid storage disease, Pick's disease, polymicrogyria (PMG), polymyositis, porencephaly, post-polio syndrome, postherpetic neuralgia (PHN), postural hypotension, Prader-Willi syndrome, primary lateral sclerosis, prion diseases, progressive hemifacial atrophy, progressive multifocal leukoencephalopathy, progressive supranuclear palsy, pseudotumor cerebri, Ramsay Hunt syndrome type I, Ramsay Hunt syndrome type II, Ramsay Hunt syndrome type III, Rasmussen's encephalitis, reflex neurovascular dystrophy, Refsum disease, restless legs syndrome, retrovirus-associated myelopathy, Rett syndrome, Reye's syndrome, rhythmic movement disorder, Romberg syndrome, Saint Vitus dance, Sandhoff disease, schizophrenia, Schilder's disease, schizencephaly, sensory integration dysfunction, septo-optic dysplasia, Shy-Drager syndrome, Sjögren's syndrome, snatiation, Sotos syndrome, spasticity, spina bifida, spinal cord tumors, spinal muscular atrophy, spinocerebellar ataxia, Steele-Richardson-Olszewski syndrome, Stiff-person syndrome, stroke, Sturge-Weber syndrome, subacute sclerosing panencephalitis, subcortical arteriosclerotic encephalopathy, superficial siderosis, Sydenham's chorea, syncope, synesthesia, syringomyelia, tarsal tunnel syndrome, tardive dyskinesia, tardive dysphrenia, Tarlov cyst, Tay-Sachs disease, temporal arteritis, tetanus, tethered spinal cord syndrome, Thomsen disease, thoracic outlet syndrome, tic douloureux, Todd's paralysis, Tourette syndrome, toxic encephalopathy, transient ischemic attack, transmissible spongiform encephalopathies, transverse myelitis, tremor, trigeminal neuralgia, tropical spastic paraparesis, trypanosomiasis, tuberous sclerosis, ubisiosis, Von Hippel-Lindau disease (VHL), Viliuisk Encephalomyelitis (VE), Wallenberg's syndrome, Werdnig, Hoffman disease, west syndrome, Williams syndrome, Wilson's disease, and Zellweger syndrome.

The compounds and compositions may also be useful in the inhibition of the development of invasive cancer, tumor angiogenesis and metastasis.

In some embodiments, the pharmaceutical composition comprises a therapeutically effective amount of a compound of Formula (I), or a pharmaceutically acceptable salt thereof, and a pharmaceutically acceptable excipient.

In some embodiments, the disorder or disease is cancer.

In some embodiments, the disorder or disease is metastatic melanoma.

In some embodiments, the disorder or disease is diabetes.

In some embodiments, the disorder or disease is a neurological disorder.

In some embodiments, the disorder or disease is Alzheimer's disease.

In some embodiments, the patient is a human.

In some embodiments, the cancer is chosen from: hepatocellular carcinoma, colon cancer, breast cancer, pancreatic cancer, chronic myeloid leukemia (CML), chronic myelomonocytic leukemia, chronic lymphocytic leukemia (CLL), acute myeloid leukemia, acute lymphocytic leukemia, Hodgkin lymphoma, lymphoma, sarcoma, and ovarian cancer.

In some embodiments, the cancer is chosen from: lung cancer—non-small cell, lung cancer—small cell, multiple myeloma, nasopharyngeal cancer, neuroblastoma, osteosarcoma, penile cancer, pituitary tumors, prostate cancer, retinoblastoma, synovial sarcoma, rhabdomyosarcoma, salivary gland cancer, skin cancer—basal and squamous cell, skin cancer—melanoma, small intestine cancer, stomach (gastric) cancers, testicular cancer, thymus cancer, thyroid cancer, uterine sarcoma, vaginal cancer, vulvar cancer, laryngeal or hypopharyngeal cancer, kidney cancer, Kaposi sarcoma, gestational trophoblastic disease, gastrointestinal stromal tumor, gastrointestinal carcinoid tumor, gallbladder cancer, eye cancer (melanoma and lymphoma), Ewing tumor, esophagus cancer, endometrial cancer, colorectal cancer, cervical cancer, brain or spinal cord tumor, bone metastasis, bone cancer, bladder cancer, bile duct cancer, anal cancer and adrenal cortical cancer.

In some embodiments, the cancer is hepatocellular carcinoma; in some embodiments, the cancer is colon cancer; in some embodiments, the cancer is colorectal cancer; in some embodiments, the cancer is breast cancer; in some embodiments, the cancer is pancreatic cancer; in some embodiments, the cancer is chronic myeloid leukemia (CML); in some embodiments, the cancer is chronic myelomonocytic leukemia; in some embodiments, the cancer is chronic lymphocytic leukemia (CLL); in some embodiments, the cancer is acute myeloid leukemia; in some embodiments, the cancer is acute lymphocytic leukemia; in some embodiments, the cancer is Hodgkin lymphoma; in some embodiments, the cancer is lymphoma; in some embodiments, the cancer is sarcoma; in some embodiments, the cancer is ovarian cancer; in some embodiments, the cancer is lung cancer—non-small cell; in some embodiments, the cancer is lung cancer—small cell; in some embodiments, the cancer is multiple myeloma; in some embodiments, the cancer is nasopharyngeal cancer; in some embodiments, the cancer is neuroblastoma; in some embodiments, the cancer is osteosarcoma; in some embodiments, the cancer is penile cancer; in some embodiments, the cancer is pituitary tumors; in some embodiments, the cancer is prostate cancer; in some embodiments, the cancer is retinoblastoma; in some embodiments, the cancer is rhabdomyosarcoma; in some embodiments, the cancer is salivary gland cancer; in some embodiments, the cancer is skin cancer—basal and squamous cell; in some embodiments, the cancer is skin cancer—melanoma; in some embodiments, the cancer is small intestine cancer; in some embodiments, the cancer is stomach (gastric) cancers; in some embodiments, the cancer is testicular cancer; in some embodiments, the cancer is thymus cancer; in some embodiments, the cancer is thyroid cancer; in some embodiments, the cancer is uterine sarcoma; in some embodiments, the cancer is vaginal cancer; in some embodiments, the cancer is vulvar cancer; in some embodiments, the cancer is Wilms tumor; in some embodiments, the cancer is laryngeal or hypopharyngeal cancer; in some embodiments, the cancer is kidney cancer; in some embodiments, the cancer is Kaposi sarcoma; in some embodiments, the cancer is gestational trophoblastic disease; in some embodiments, the cancer is gastrointestinal stromal tumor; in some embodiments, the cancer is gastrointestinal carcinoid tumor; in some embodiments, the cancer is gallbladder cancer; in some embodiments, the cancer is eye cancer (melanoma and lymphoma); in some embodiments, the cancer is Ewing tumor; in some embodiments, the cancer is esophagus cancer; in some embodiments, the cancer is endometrial cancer; in some embodiments, the cancer is colorectal cancer; in some embodiments, the cancer is cervical cancer; in some embodiments, the cancer is brain or spinal cord tumor; in some embodiments, the cancer is bone metastasis; in some embodiments, the cancer is bone cancer; in some embodiments, the cancer is bladder cancer; in some embodiments, the cancer is bile duct cancer; in some embodiments, the cancer is anal cancer; and in some embodiments, the cancer is adrenal cortical cancer.

In some embodiments, the disorder or disease is a neurological condition, disorder, or disease, wherein the neurological disease is selected from: Alzheimer's disease, frontotemporal dementias, Parkinson's disease, Huntington's disease, progressive supranuclear palsy, corticobasal degeneration, multiple system atrophy, amyotrophic lateral sclerosis (ALS), inclusion body myositis, autism, degenerative myopathies.

In some embodiments, the disorder or disease is selected from the group consisting of: Alzheimer's Disease, Amyotrophic Lateral Sclerosis, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, and Stroke.

In some embodiments, a compound of Formula (I) inhibits DYRK1A.

In some embodiments, the method treats a disease or disorder mediated by kinase activity in a patient, the method comprises administering to the patient a therapeutically effective amount of a compound (or compounds) of Formula (I), or a pharmaceutically acceptable salt thereof.

In some embodiments, the disease or disorder comprises tumor growth, cell proliferation, or angiogenesis.

In some embodiments, the method inhibits the activity of a protein kinase receptor, the method comprises contacting the receptor with an effective amount of a compound (or compounds) of Formula (I), or a pharmaceutically acceptable salt thereof.

In some embodiments, the method treats a disease or disorder associated with aberrant cellular proliferation in a patient; the method comprises administering to the patient a therapeutically effective amount of a compound (or compounds) of Formula (I), or a pharmaceutically acceptable salt thereof.

In some embodiments, the method prevents or reduces abnormal cellular proliferation in a patient; the method comprises administering to the patient a therapeutically effective amount of a compound (or compounds) of Formula (I), or a pharmaceutically acceptable salt thereof.

In some embodiments, the method treats a disease or disorder associated with aberrant cellular proliferation in a patient, the method comprises administering to the patient a pharmaceutical composition comprising one or more of the compounds of claim 1 in combination with a pharmaceutically acceptable carrier and one or more other agents.

### Evaluation of Biological Activity

The biological activity of the compounds described herein can be tested using any suitable assay known to those of skill in the art. For example, the activity of a compound may be tested using one or more of the test methods outlined below.

For example, in vitro assays for DYRK1A biological activity may be used, e.g., regulation of microtubule-associated protein tau (MAPT/Tau) phosphorylation in neuronal cell lines such as the human SH-SY5Y neuroblastoma cell line. Assays for DYRK1A-regulated level of phosphorylation can include monitoring levels of basal pSer396 Tau, which can be measured, for example, by serial dilutions of a candidate inhibitor composition using a ten micromolar top concentration and detected by ELISA or Western Blotting. An exemplary assay for DYRK-1A-regulated phosphorylation uses the SH-SY5Y cells cultured in a 96 well plate format for a period of time sufficient to stabilize microtubules and Tau phosphorylation, usually at least 2 days, then treated with a ⅓ serial dilution of compounds overnight and lysed. The cell lysate is resolved by SDS PAGE, then transferred to nitrocellulose and probed with an antibody specific for pSer396 Tau. The chemiluminescence signal for HRP-linked antibodies used in western blotting is detected using a Carestream Image Station and blot densitometry for pSer396 and beta-actin are analyzed using ImageJ (NIH).

In a further example, the activity of a candidate compound can be measured by phosphoTau (Thr212) AlphaLISA by adding the lysate mentioned above onto total Tau-coated plates and detected with a specific pThr212Tau antibody. Colorimetric detection of AlphaLISA signal is performed by EnVision Multilabel Plate Reader (Perkin Elmer).

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described and claimed herein. The reader will recognize that the skilled artisan, armed with the present disclosure, and skill in the art is able to prepare and use the disclosure without exhaustive examples.

### EXAMPLES

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

(¹H) nuclear magnetic resonance spectra (NMR) were measured in the indicated solvents on a Bruker NMR spectrometer (Avance™ DRX300, 300 MHz for ¹H or Avance™ DRX500, 500 MHz for ¹H) or Varian NMR spectrometer (Mercury 400BB, 400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; ddd, doublet of doublets of doublets; d/ABq, doublet of AB quartet; dt, doublet of triplets; td, triplet of doublets; dq, doublet of quartets; m, multiplet.

The following abbreviations have the indicated meanings:
Ac₂O=acetic anhydride
Boc=tert-butyloxycarbonyl
BrettPhos=dicyclohexyl(2′,4′,6′-triisopropyl-3,6-dimethoxy-[1,1′-biphenyl]-2-yl) phosphine
BrettPhos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
brine=saturated aqueous sodium chloride
CDCl₃=deuterated chloroform
Cs₂CO₃=cesium carbonate
DCE=1,2-dichloroethane
DCM=dichloromethane
DIPEA=N,N-diisopropylethylamine
DME=1,2-dimethoxyethane, or glyme, or monoglyme
DMF=N,N-dimethylformamide
DMPU=N,N′-dimethylpropyleneurea
DMSO-d₆=deuterated dimethylsulfoxide
ESIMS=electron spray mass spectrometry
EtOAc=ethyl acetate
HATU=1-[bis(dimethylamino)methylene]-1H-1,2,3-triazolo[4,5-b]pyridinium 3-oxid
hexafluorophosphate
HCl=hydrochloric acid
HOAc=acetic acid
ISCO=Teledyne ISCO, Inc brand CombiFlash® Rf 200
KOAc=potassium acetate
LCMS=liquid chromatography-mass spectrometry
LiHMDS=lithium bis(trimethylsilyl)amide
MeCN=acetonitrile
MeOH=methanol
MeTHF=2-methyltetrahydrofuran
MgSO₄=magnesium sulfate
MTBE=methyl tert-butyl ether
MW=microwave irradiation
NaO^{t}Bu=Sodium tert-butoxide
NaHCO₃=sodium bicarbonate
NaBH₃CN=sodium cyanoborohydride
NaBH(OAc)₃=Sodium triacetoxyborohydride
Na₂SO₄=sodium sulfate
NMR=nuclear magnetic resonance
ON=overnight
Pd/C=palladium on carbon
Pd(dppf)Cl₂=1,1′-bis(diphenylphosphino)ferrocene-palladium(II)dichloride
Pd(OAc)₂=palladium(II) acetate
Pd(OH)₂/C=palladium hydroxide on carbon
PE=petroleum ether
prep-TLC=preparative thin layer chromatography
r.t.=room temperature
TEA=triethylamine
TFA=trifluoroacetic acid
THF=tetrahydrofuran
TLC=thin layer chromatography
TMSOK=potassium trimethylsilanolate
XPhos=dicyclohexyl[2′,4′,6′-tris(propan-2-yl)[1,1′-biphenyl]-2-yl]phosphane

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only and should not be construed as or confused with same numberings in other sections of the application. Unless otherwise indicated, all variables are as defined above.

### General Procedures

Compounds of Formula I of the present disclosure can be prepared as depicted in Scheme 1.

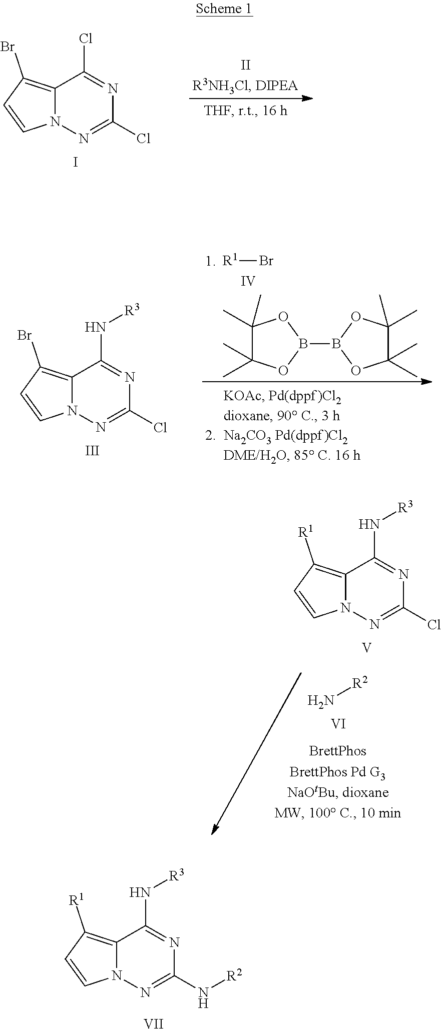

Scheme 1 describes a method for preparation of 4-alkylaminopyrrolo[2,1-f][1,2,4]triazine derivatives (VII) by first displacing the 4-chloride (I) with a variety of amines (II) to produce 2-chloro-5-bromo-4-aminopyrrolo[2,1-f][1,2,4]triazine III. Formation of a variety of boronic acid pinacol esters by reacting various bromides (IV) with bis(pinacolato)diboron followed by Suzuki coupling with bromide (III) produces 2-chloro-4-aminopyrrolo[2,1-f][1,2,4]triazine (V). The chloro is then displaced with a variety of amines (VI) to produce the final 4-aminopyrrolo[2,1-f][1,2,4]triazine (VII).

Compounds of Formula I of the present disclosure can also be prepared as depicted in Scheme 2.

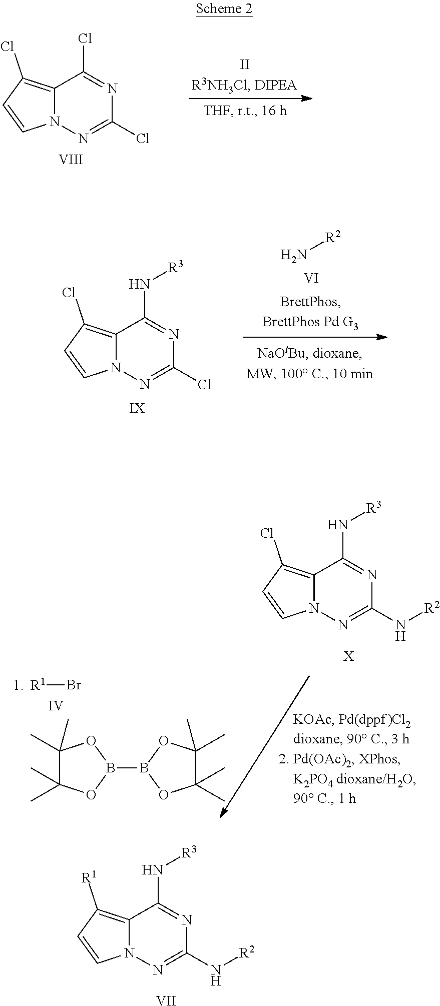

Scheme 2 describes a method for preparation of 4-aminopyrrolo[2,1-f][1,2,4]triazine derivatives (VII) by first displacing the 4-chloride (VIII) with a variety of amines (II) to produce 2,5-dichloro-4-aminopyrrolo[2,1-f][1,2,4]triazine IX. The 2-chloro is then displaced with a variety of amines (VI) to produce 5-chloro-4-aminopyrrolo[2,1-f][1,2,4]triazine (X). Formation of a variety of boronic acid pinacol esters by reacting various bromides (IV) with bis(pinacolato)diboron followed by Suzuki coupling with chloride (X) produces the final 4-aminopyrrolo[2,1-f][1,2,4]triazine (VII).

### Illustrative Compound Examples

Preparation of intermediate 5-bromo-2-chloro-N-methylpyrrolo[2,1-f][1,2,4]triazin-4-amine (XV) is depicted below in Scheme 3.

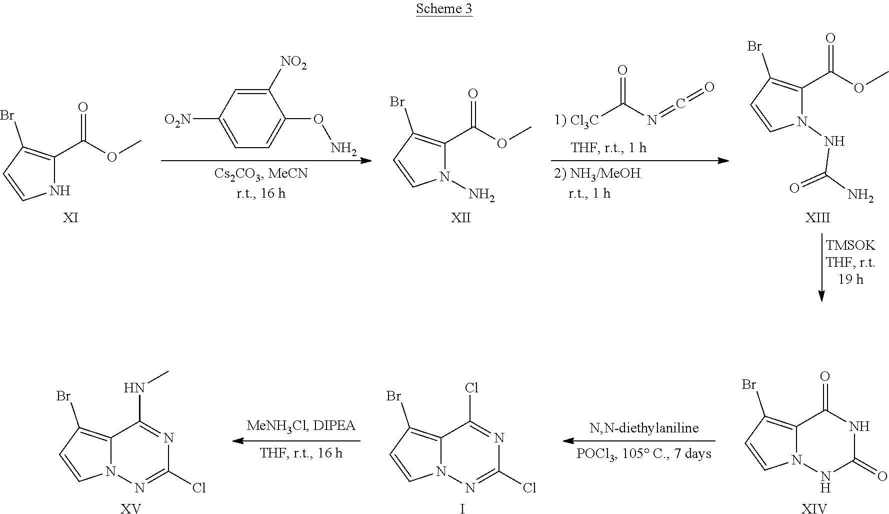

### Step 1

To a solution of methyl 3-bromo-1H-pyrrole-2-carboxylate (XI) (100 g, 490.15 mmol, 1 eq.) in MeCN (2 L) was added Cs₂CO₃ (255.52 g, 784.23 mmol, 1.6 eq.) in one portion at 20° C. (no exothermic). The reaction was stirred at room temperature for 5 h. A solution of O-(2,4-dinitrophenyl)hydroxylamine (146.40 g, 735.22 mmol, 1.5 eq.) in MeCN (2 L) was then added dropwise at 0-5° C. under N₂. After addition, the reaction was warmed to room temperature for 16 h. The reaction mixture was filtered, and the filtrate was diluted with MTBE (2 L) and washed with brine (2 L×3). The above reaction was performed six times.

The combined organics of five batches were combined, dried over MgSO₄, filtered, and concentrated under vacuum to give the crude product. The crude product was purified by column chromatography on silica gel (DCM/PE=0→50%, then EtOAc/PE=20%) to give methyl 1-amino-3-bromo-1H-pyrrole-2-carboxylate (XII) (380 g, 1.62 mol, 55.1% yield, 93.3% purity) as a yellow solid. ¹H NMR (400 MHz, DMSO-d₆) δ ppm 3.84 (3H, s), 6.24 (1H, d, J=2.8 Hz), 6.38 (2H, br s), 7.12 (1H, d, J=2.8 Hz).

### Steps 2-3

To a solution of methyl 1-amino-3-bromo-1H-pyrrole-2-carboxylate (XII) (190 g, 867.44 mmol, 1 eq.) in THF (1900 mL) was added dropwise 2,2,2-trichloroacetyl isocyanate (179.76 g, 954.18 mmol, 113.06 mL, 1.1 eq.) at 0˜5° C., then warmed to room temperature for 1 h. The reaction was then added dropwise to NH₃/MeOH (7 M, 1.24 L, 10 eq) at room temperature, stirred at room temperature for 1 h to give a yellow suspension. The above reaction was performed twice.

The two reactions were combined and concentrated under vacuum. The residue was triturated with MTBE (4 L) at room temperature for 30 min, filtered. The filter cake was washed with MTBE (500 mL×3), dried under vacuum to give methyl 3-bromo-1-ureido-1H-pyrrole-2-carboxylate (XIII) (402 g, 1.53 mol, 88.2% yield, 99.78% purity) as an off-white solid. ¹H NMR (400 MHz, DMSO-d₆) δ ppm 3.75 (3H, s), 6.27 (2H, br s), 6.28 (1H, d, J=2.8 Hz), 7.10 (1H, d, J=2.8 Hz), 9.27 (1H, br s); ESIMS found for C₇H₈BrN₃O₃ m/z 262.1 (⁷⁹BrM+H).

### Step 4

To a solution of methyl 3-bromo-1-ureido-1H-pyrrole-2-carboxylate (XIII) (67 g, 255.67 mmol, 1 eq.) in THF (4 L) was added TMSOK (65.60 g, 511.33 mmol, 2 eq.) in portions at 0-5° C. under N₂. After stirring for 5 min, the reaction became a thick slurry. The reaction mixture was stirred vigorously at room temperature for 16 h. LCMS showed ˜25.8% of starting material remained. An additional portion of TMSOK (16.40 g, 127.83 mmol, 0.5 eq.) was added at room temperature and stirred for 3 h. LCMS showed the reaction was complete. The reaction was concentrated. The residue was diluted with H₂O (1.6 L), cooled with ice-bath, added dropwise HCl (4 M, 160 mL) to pH 2-3, and filtered. The filter cake was washed with H₂O (600 mL×3). The above reaction was performed six times.

The filter cakes of six reactions were combined, triturated with H₂O (6 L) at room temperature, for 2 h and then filtered. The filter cake was dried in a vacuum drying oven at 60° C. for 24 h to give 5-bromopyrrolo[2,1-f][1,2,4]triazine-2,4(1H,3H)-dione (XIV) (320 g, 1.28 mol, 83.4% yield, 91.95% purity) as a white solid. ¹H NMR (400 MHz, DMSO-d₆) δ ppm 6.49 (1H, d, J=2.8 Hz), 7.17 (1H, d, J=2.8 Hz), 11.30 (1H, br s); ESIMS found for C₆H₄BrN₃O₂ m/z 232.0 (⁸¹BrM+H).

### Step 5

To a solution of POCl₃ (1500 mL) was added 5-bromopyrrolo[2,1-f][1,2,4]triazine-2,4(1H,3H)-dione (XIV) (180 g, 782.55 mmol, 1 eq.) in portions at room temperature. N,N-Diethylaniline (291.95 g, 1.96 mol, 312.92 mL, 2.5 eq.) was then added dropwise at room temperature (a little exothermic). After addition, the reaction was heated to 105° C. for 48 h. LCMS showed the starting material was consumed but the intermediate remained, ˜4.3% of desired product was formed. The reaction was cooled to 90° C., added an additional POCl₃ (500 mL) in one portion, then heated to reflux gently for 24 h. LCMS showed most of intermediate remained, ˜11.9% of desired product was formed. The reaction was heated to reflux gently for another 4 days. LCMS showed ˜7.4% of intermediate remained, ˜55.5% of desired product was formed. The reaction was cooled to 40° C. and distilled under reduced pressure to remove most of POCl₃. The residue was diluted with MeTHF (5 L), poured into ice-H₂O (2 L), the added brine (1 L), and separated. The aqueous layer was extracted with MeTHF (1.5 L×2). The combined organic were washed with brine (1.5 L×2), dried over MgSO₄, filtered, and concentrated under vacuum to give the crude product. The crude product was purified by column chromatography on silica gel (4 kg, 100˜200 mesh, DCM/PE=0-20%) and triturated with n-heptane (500 mL) at room temperature for 3 h, then filtered to afford 5-bromo-2,4-dichloropyrrolo[2,1-f][1,2,4]triazine (I) (150 g, 556.14 mmol, 71.1% yield, 98.96% purity) as a yellow solid. ¹H NMR (400 MHz, CDCl₃) δ ppm 7.02 (1H, d, J=2.4 Hz), 7.79 (1H, d, J=2.4 Hz); ESIMS found for C₆H₂BrCl₂N₃ m/z 266.1 (⁷⁹BrM+H).

### Step 6

To a stirred solution of 5-bromo-2,4-dichloropyrrolo[2,1-f][1,2,4]triazine (I) (10 g, 37.47 mmol) in THF (250 mL) was added methylammonium chloride (6.33 g, 93.75 mmol) and DIPEA (22.9 mL, 131.47 mmol). The mixture was stirred at room temperature for 16 h. The reaction mixture was quenched with water, extracted in EtOAc, washed with brine and dried over anhydrous Na₂SO₄. The solvent was evaporated on rotavapor, and the crude mixture was purified on ISCO (0→10% MeOH/CHCl₃) to obtain 5-bromo-2-chloro-N-methylpyrrolo[2,1-f][1,2,4]triazin-4-amine (XV) (8.9 g, 34.033 mmol, 90.8% yield) as a white solid. ESIMS found for C₇H₆BrClN₄ m/z 261.0 (M+H).

The following intermediates were prepared in accordance with the procedure described in the above Scheme 3.

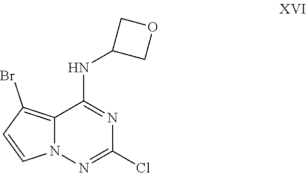 [SMILES: Clc1nc(NC2COC2)c2c(Br)ccn2n1]

5-Bromo-2-chloro-N-(oxetan-3-yl)pyrrolo[2,1-f][1,2,4]triazin-4-amine (XVI): White solid (461 mg, 1.519 mmol, 81.1% yield). ESIMS found C₉H₈BrClN₄O m/z 303.0 (M+H).

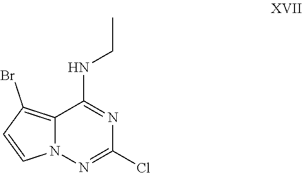

5-Bromo-2-chloro-N-ethylpyrrolo[2,1-f][1,2,4]triazin-4-amine (XVII): Beige solid (503.6 mg, 1.831 mmol, 97.6% yield). ESIMS found C₈H₈BrClN₄ m/z 275.0 (M+H).

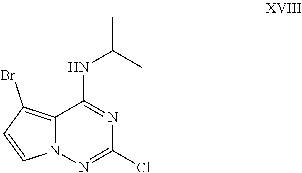

5-Bromo-2-chloro-N-isopropylpyrrolo[2,1-f][1,2,4]triazin-4-amine (XVIII): White solid (453.0 g, 1.564 mmol, 83.5% yield). ESIMS found C₉H₁₀BrClN₄ m/z 289.0 (M+H).

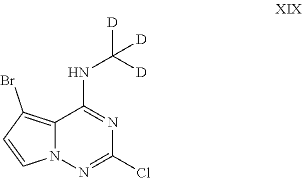 [SMILES: [2H]C([2H])([2H])Nc1nc(Cl)nn2ccc(Br)c12]

5-Bromo-2-chloro-N-(methyl-d₃)pyrrolo[2,1-f][1,2,4]triazin-4-amine (XIX): Orange solid (647 mg, 2.446 mmol, 21.8% yield). ESIMS found C₇H₃D₃BrClN₄ m/z 264.0 (M+H).

Preparation of intermediate 5-bromo-2-chloropyrrolo[2,1-f][1,2,4]triazin-4-amine (XX) is depicted below in Scheme 4.

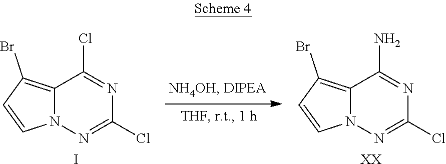

### Step 1

To a stirred solution of ammonium hydroxide (4.7 mL, 37.47 mmol) in THF (10 mL) was added 5-bromo-2,4-dichloropyrrolo[2,1-f][1,2,4]triazine (I) (1 g, 3.75 mmol) and the mixture was stirred at room temperature for 1 h. The reaction mixture was extracted in DCM, washed with brine, and dried over anhydrous Na₂SO₄. The solvent was evaporated on rotavapor and dried under high vacuo to obtain 5-bromo-2-chloropyrrolo[2,1-f][1,2,4]triazin-4-amine (XX) (900 mg, 3.637 mmol, 97.1% yield) as off-white solid. ESIMS found for C₆H₄BrClN₄ m/z 246.95 (M+H).

The following intermediate was prepared in accordance with the procedure described in the above Scheme 4.

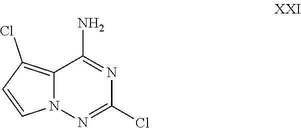 [SMILES: Nc1nc(Cl)nn2ccc(Cl)c12]

2,5-Dichloropyrrolo[2,1-f][1,2,4]triazin-4-amine (XXI): Pale-orange solid (308 mg, 1.517 mmol, 16.9% yield). ESIMS found C₆H₄Cl₂N₄ m/z 203.0 (M+H).

Preparation of intermediate 2,5-dichloro-N-methylpyrrolo[2,1-f][1,2,4]triazin-4-amine (XXII) is depicted below in Scheme 5.

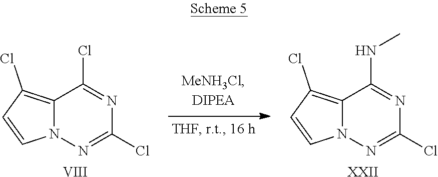

### Step 1

To a stirred solution of 2,4,5-trichloropyrrolo[2,1-f][1,2,4]triazine (commercially available from PharmaBlock Sciences Inc.) (VIII) (2 g, 8.99 mmol) in THF (50 mL) were added methylammonium chloride (1.51 g, 22.36 mmol) and DIPEA (5.48 mL, 31.46 mmol) at −78° C. and the mixture was stirred at room temperature for 16 h. The reaction mixture was quenched with saturated NaHCO₃ solution, extracted with DCM, washed with brine, and dried over anhydrous Na₂SO₄. The solvent was evaporated on rotavapor and dried under high vacuo to obtain 2,5-dichloro-N-methylpyrrolo[2,1-f][1,2,4]triazin-4-amine (XXII) (1.92 g, 8.846 mmol, 98.4% yield) as a yellow solid. ESIMS found for C₇H₆Cl₂N₄ m/z 217.0 (M+H).

Preparation of intermediate 6-bromo-1-(2,2-difluoroethyl)-2-methyl-1H-imidazo[4,5-b]pyridine (XXVII) is depicted below in Scheme 6.

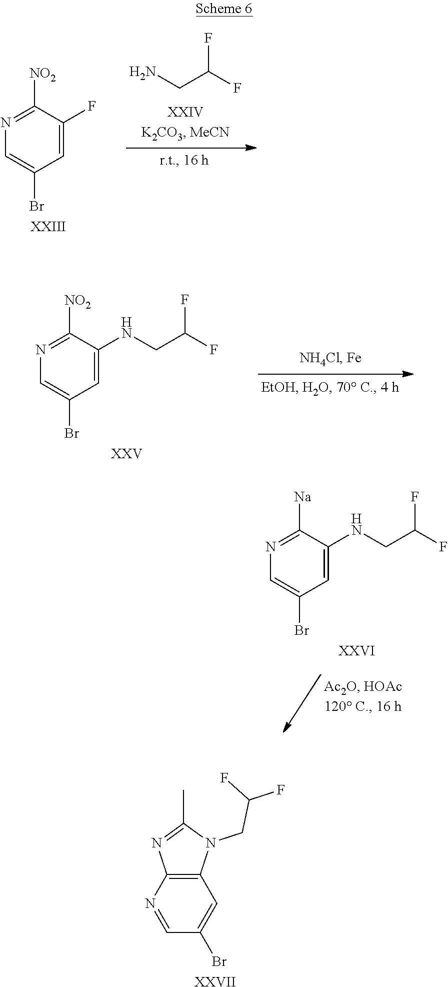

### Step 1

A mixture of 2,2-difluoroethan-1-amine (XXIV) (410 mg, 5.02 mmol), 5-bromo-3-fluoro-2-nitropyridine (XXIII) (commercially available from Ark Pharma Scientific Limited) (1.0 g, 4.53 mmol) and K₂CO₃ (1.38 g, 9.95 mmol) in MeCN (20 mL) was stirred at room temperature for 16 h. The reaction was filtered and concentrated under high vacuum. The residue was taken up in water, stirred for 1 hour and the solids were collected by filtration and dried in vacuo to obtain 5-bromo-N-(2,2-difluoroethyl)-2-nitropyridin-3-amine (XXV) (1.066 g, 3.780 mmol, 83.5% yield) as a yellow solid which was used for next step without purification. ESIMS found for C₇H₆BrF₂N₃O₂ m/z 282.0 (⁷⁹BrM+H).

### Step 2

A mixture of 5-bromo-N-(2,2-difluoroethyl)-2-nitropyridin-3-amine (XXV) (1.32 g, 4.69 mmol), Fe (3.07 g, 46.95 mmol) and NH₄Cl (3.77 g, 70.48 mmol) was taken in a mixture of EtOH (18 mL), and water (6 mL) and the mixture was heated to 70° C. for 4 h. The reaction mixture was cooled and filtered through Celite®. The filtrates were taken up in EtOAc, washed with water and brine, dried over anhydrous Na₂SO₄, filtered, and concentrated under reduced pressure to obtain 5-bromo-N³-(2,2-difluoroethyl)pyridine-2,3-diamine (XXVI) (630 mg, 2.499 mmol, 53.2% yield) as a grey solid which was used for next reaction without further purification. ESIMS found for C₇H₈BrF₂N₃ m/z 252.0 (⁷⁹BrM+H).

### Step 3

A solution of 5-bromo-N³-(2,2-difluoroethyl)pyridine-2,3-diamine (XXVI) (630 mg, 2.5 mmol) and acetic anhydride (0.28 mL, 2.97 mmol) in HOAc (15 mL) was heated to 120° C. for 16 h. The reaction mixture was concentrated, the residue partitioned between EtOAc/1 N NaOH, organics separated, and washed with water and brine. The organics were dried over anhydrous Na₂SO₄, solvents and concentrated under high vacuum. The residue was triturated with diethyl ether, sonicated and the solids were collected by filtration and dried under high vacuo to obtain 6-bromo-1-(2,2-difluoroethyl)-2-methylimidazo[4,5-b]pyridine (XXVII) (325 mg, 1.177 mmol, 47.10% yield) as a grey solid which was used for next step without purification. ESIMS found for C₉H₈BrF₂N₃ m/z 276.0 (⁷⁹BrM+H).

The following intermediate was prepared in accordance with the procedure described in the above Scheme 6.

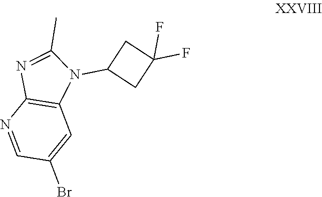 [SMILES: Cc1nc2ncc(Br)cc2n1C1CC(F)(F)C1]

6-Bromo-1-(3,3-difluorocyclobutyl)-2-methyl-1H-imidazo[4,5-b] pyridine (XXVIII): Grey solid (1.57 g, 6.178 mmol, 68.3% yield). ESIMS found C₁₁H₁₀BrF₂N₃ m/z 302.1 (M+H).

Preparation of intermediate 6-bromo-1-isopropyl-2-methyl-1H-imidazo[4,5-b]pyridine (XXXII) is depicted below in Scheme 7.

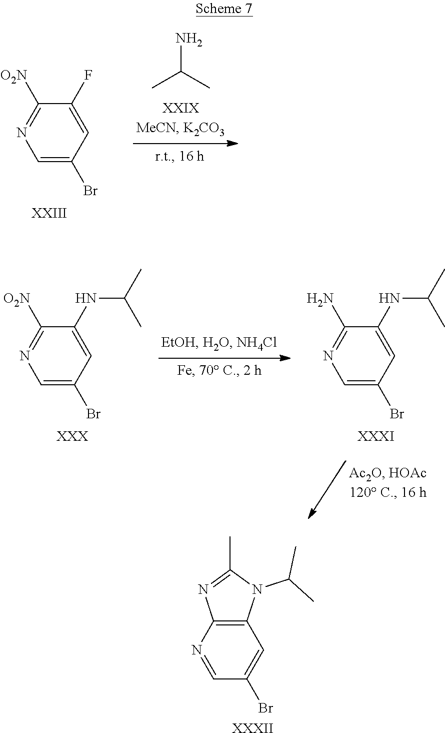

### Step 1

A mixture of 2-aminopropane (XXIX) (0.86 mL, 9.96 mmol), 5-bromo-3-fluoro-2-nitropyridine (XXIII) (2 g, 9.05 mmol) and K₂CO₃ (2.5 g, 18.1 mmol) in MeCN (40 mL) was stirred at room temperature for 16 h. The reaction mixture was added to water (200 mL), stirred for 1 h and the resulting solids were collected by filtration and dried under high vacuo to obtain 5-bromo-N-isopropyl-2-nitropyridin-3-amine (XXX) (2.36 g, 9.074 mmol, 100.3% yield) as a yellow solid which was used for next step without purification. ESIMS found for C₈H₁₀BrN₃O₂ m/z 260.0 (M+H).

### Step 2

A mixture of 5-bromo-N-isopropyl-2-nitropyridin-3-amine (XXX) (2.35 g, 9.04 mmol) Fe (5.91 g, 90.35 mmol) and NH₄Cl (7.25 g, 135.53 mmol) was taken in a mixture of EtOH (30 mL) and water (10 mL) and the mixture was heated to 70° C. for 2 h. The reaction mixture was cooled, filtered through Celite®, filtrates were taken into EtOAc, washed with water then brine, dried over anhydrous Na₂SO₄, filtered, and concentrated under reduced pressure to obtain 5-bromo-N³-isopropylpyridine-2,3-diamine (XXXI) (2.2 g, 9.561 mmol, 105.8% yield) as a dark brown solid which was used for next step without purification. ESIMS found for C₈H₁₂BrN₃ m/z 230.05 (M+H).

### Step 3

A solution of 5-bromo-N³-isopropylpyridine-2,3-diamine (XXXI) (2.08 g, 9.04 mmol) and Ac₂O (1.05 mL, 10.84 mmol) in HOAc (20 mL) was heated to 120° C. for 16 h. The reaction mixture was concentrated, the residue partitioned between EtOAc/1 N NaOH, organics separated, washed with water and brine. The organics were dried over anhydrous Na₂SO₄, solvents concentrated and dried under high vacuo to give 6-bromo-1-isopropyl-2-methyl-1H-imidazo[4,5-b]pyridine (XXXII) (1.57 g, 6.178 mmol, 68.3% yield) as a dark brown solid which was used for next step without purification. ESIMS found for C₁₀H₁₂BrN₃ m/z 254.0 (M+H).

Preparation of intermediate 5-chloro-3-ethyl-2-methyl-3H-imidazo[4,5-b]pyridine (XXXIV) is depicted below in Scheme 8.

### Step 1

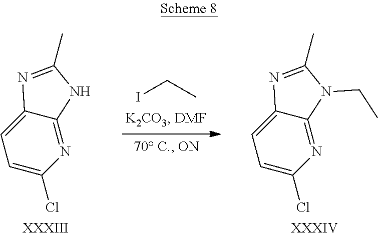

A mixture of 5-chloro-2-methyl-3H-imidazo[4,5-b]pyridine (XXXIII) (commercially available from eNovation Chemicals, LLC) (0.5 g, 2.98 mmol), iodoethane (0.56 g, 3.58 mmol) and K₂CO₃ (0.83 g, 5.97 mmol) in DMF (10 mL) was heated to 70° C. overnight. The reaction mixture was cooled, solvents concentrated, the residue partitioned between EtOAc/water, the organic layers were separated, washed with brine, dried over anhydrous MgSO₄ and the solvents were concentrated under vacuo and dried to obtain 5-chloro-3-ethyl-2-methylimidazo[4,5-b]pyridine (XXXIV) (466 mg, 2.382 mmol, 79.8% yield) as a dark brown solid which was used for next step without purification. ESIMS found for C₉H₁₀ClN₃ m/z 196.05 (M+H).

The following intermediates were prepared in accordance with the procedure described in the above Scheme 8.

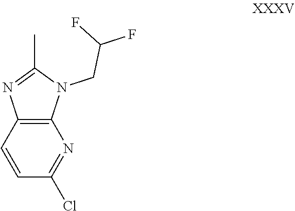

5-Chloro-3-(2,2-difluoroethyl)-2-methyl-3H-imidazo[4,5-b]pyridine (XXXV): Beige solid (830 mg, 3.583 mmol, 60.1% yield). ESIMS found C₉H₈ClF₂N₃ m/z 232.0 (M+H).

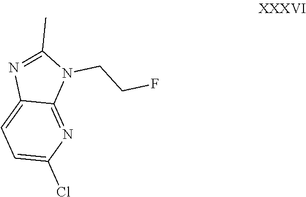

5-Chloro-3-(2-fluoroethyl)-2-methyl-3H-imidazo[4,5-b]pyridine (XXXVI): Beige solid (220 mg, 1.030 mmol, 57.5% yield). ESIMS found C₉H₉ClFN₃ m/z 214.05 (M+H).

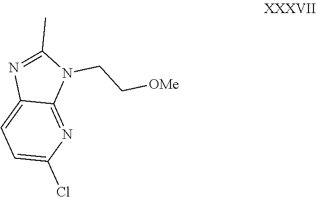

5-Chloro-3-(2-methoxyethyl)-2-methyl-3H-imidazo[4,5-b]pyridine (XXXVII): Beige solid (195 mg, 0.864 mmol, 48.3% yield). ESIMS found C₁₀H₁₂ClN₃O m/z 226.1 (M+H).

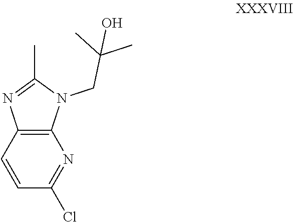 [SMILES: Cc1nc2ccc(Cl)nc2n1CC(C)(C)O]

1-(5-Chloro-2-methyl-3H-imidazo[4,5-b]pyridin-3-yl)-2-methylpropan-2-ol (XXXVIII): White solid (229.9 mg, 0.959 mmol, 39.7% yield). H NMR (499 MHz, DMSO-d₆) δ ppm 1.13 (6H, s), 2.63 (3H, s), 4.11 (2H, s), 4.80 (1H, s), 7.25 (1H, d, J=8.21 Hz), 7.96 (1H, d, J=8.21 Hz); ESIMS found C₁₁H₁₄ClN₃O m/z 240.1 (M+H).

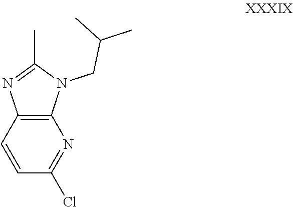

5-Chloro-3-isobutyl-2-methyl-3H-imidazo[4,5-b]pyridine (XXXIX): White solid (206.8 mg, 0.925 mmol, 38.7% yield). ¹H NMR (499 MHz, DMSO-d₆) δ ppm 0.87 (6H, d, J=6.57 Hz), 2.22 (1H, dquin, J=13.89, 7.07, 7.07, 7.07, 7.07 Hz), 2.58 (3H, s), 4.01 (2H, d, J=7.67 Hz), 7.26 (1H, d, J=8.21 Hz), 7.98 (1H, d, J=8.21 Hz); ESIMS found C₁₁H₁₄ClN₃ m/z 224.1 (M+H).

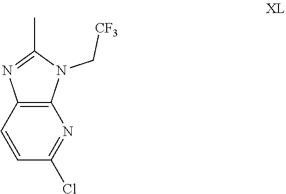 [SMILES: Cc1nc2ccc(Cl)nc2n1CC(F)(F)F]

5-Chloro-2-methyl-3-(2,2,2-trifluoroethyl)-3H-imidazo[4,5-b]pyridine (XL): Beige solid (372 mg, 1.490 mmol, 50.0% yield). ESIMS found C₉H₇ClF₃N₃ m/z 250.0 (M+H).

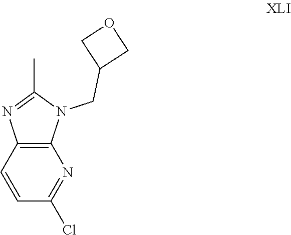 [SMILES: Cc1nc2ccc(Cl)nc2n1CC1COC1]

5-Chloro-2-methyl-3-(oxetan-3-ylmethyl)-3H-imidazo[4,5-b]pyridine (XLI): Light brown solid (289.7 mg, 1.219 mmol, 40.4% yield). ¹H NMR (499 MHz, DMSO-d₆) δ ppm 2.59 (3H, s), 3.43-3.56 (1H, m), 4.47 (2H, t, J=6.02 Hz), 4.52 (2H, d, J=7.67 Hz), 4.62 (2H, dd, J=7.67, 6.02 Hz), 7.27 (1H, d, J=8.21 Hz), 7.98 (1H, d, J=8.21 Hz); ESIMS found C₁₁H₁₂ClN₃O m/z 238.1 (M+H).

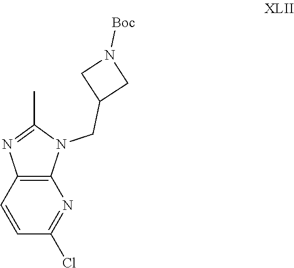 [SMILES: Cc1nc2ccc(Cl)nc2n1CC1CN(C(=O)OC(C)(C)C)C1]

tert-Butyl 3-((5-chloro-2-methyl-3H-imidazo[4,5-b]pyridin-3-yl)methyl) azetidine-1-carboxylate (XLII): Off-white amorphous solid (532.6 mg, 1.581 mmol, 52.1% yield). ESIMS found C₁₆H₂₁ClN₄O₂ m/z 337.1 (M+H).

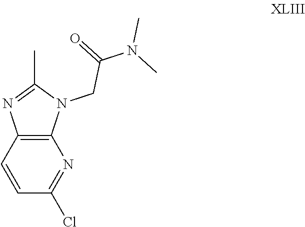 [SMILES: Cc1nc2ccc(Cl)nc2n1CC(=O)N(C)C]

2-(5-Chloro-2-methyl-3H-imidazo[4,5-b]pyridin-3-yl)-N,N-dimethylacetamide (XLIII): Off-white solid (474.4 mg, 1.877 mmol, 62.6% yield). ESIMS found C₁₁H₁₃ClN₄O m/z 253.1 (M+H).

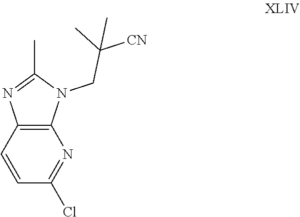 [SMILES: Cc1nc2ccc(Cl)nc2n1CC(C)(C)C#N]

3-(5-Chloro-2-methyl-3H-imidazo[4,5-b]pyridin-3-yl)-2,2-dimethylpropanenitrile (XLIV): Off-white amorphous solid (59.1 mg, 0.238 mmol, 7.9% yield). ESIMS found C₁₂H₁₃ClN₄ m/z 249.1 (M+H).

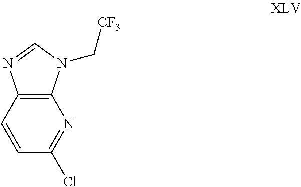 [SMILES: FC(F)(F)Cn1cnc2ccc(Cl)nc21]

5-Chloro-3-(2,2,2-trifluoroethyl)-3H-imidazo[4,5-b]pyridine (XLV): Light yellow solid (335 mg, 1.422 mmol, 21.8% yield). ESIMS found C₈H₅ClF₃N₃ m/z 236.0 (M+H).

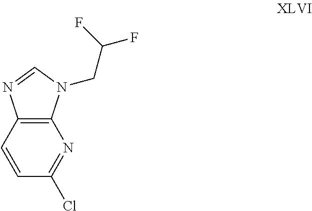 [SMILES: FC(F)Cn1cnc2ccc(Cl)nc21]

5-Chloro-3-(2,2-difluoroethyl)-3H-imidazo[4,5-b]pyridine (XLVI): White solid (253 mg, 1.163 mmol, 20.5% yield). ESIMS found C₈H₆ClF₂N₃ m/z 218.0 (M+H).

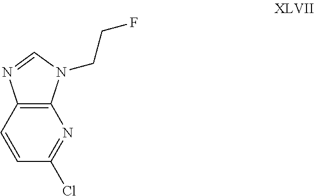 [SMILES: FCCn1cnc2ccc(Cl)nc21]

5-Chloro-3-(2-fluoroethyl)-3H-imidazo[4,5-b]pyridine (XLVII): Beige solid (470 mg, 2.355 mmol, 36.2% yield). ESIMS found C₈H₇ClFN₃ m/z 200.05 (M+H).

Preparation of intermediate 5-chloro-3-(2,2-difluoroethyl)-3H-[1,2,3]triazolo[4,5-b]pyridine (LI) is depicted below in Scheme 9.

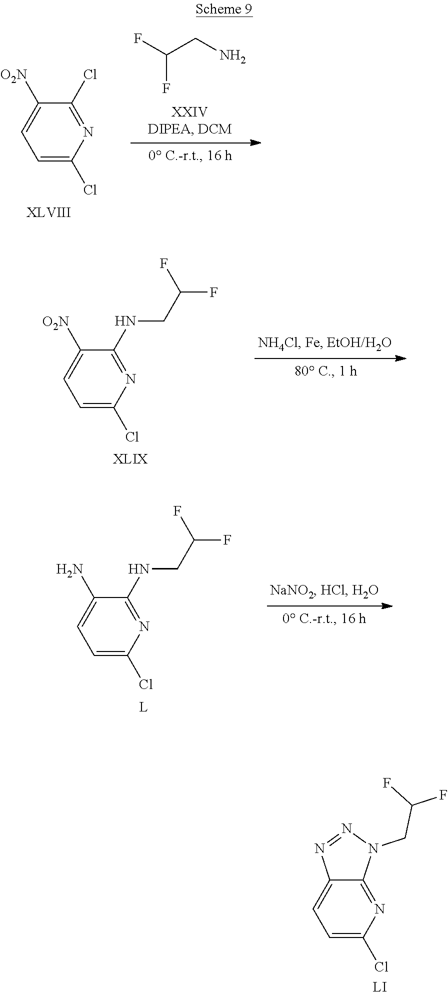

### Step 1

To a solution of 2,6-dichloro-3-nitropyridine (XLVIII) (14.0 g, 73.30 mmol) in DCM (500.0 mL) cooled to 0° C. was added 2,2-difluoroethan-1-amine (XXIV) (8.91 g, 109.95 mmol) at 0° C. DIPEA (18.91 g, 146.60 mmol) was then added, and the reaction was warmed to room temperature for 16 h. The reaction mixture was extracted with DCM (500 mL×2). The combined organics were washed with brine (500 mL×3), dried over anhydrous Na₂SO₄, filtered, and concentrated to give the crude product. The crude was purified by silica gel column chromatography (PE/EtOAc=10:1→5:1) to give 6-chloro-N-(2,2-difluoroethyl)-3-nitropyridin-2-amine (XLIX) (12.0 g, 50.507 mmol, 68.9%) as a yellow solid. ¹H NMR (400 MHz, DMSO-d₆) δ 4.01-3.93 (m, 2H), 6.43-6.03 (m, 1H), 6.95-6.79 (m, 1H), 8.54-8.40 (m, 1H), 8.81 (t, J=5.4 Hz, 1H).

### Step 2

6-Chloro-N-(2,2-difluoroethyl)-3-nitropyridin-2-amine (XLIX) (12.0 g, 50.51 mmol) was added to a mixture of NH₄Cl (7.37 g, 139.00 mmol) in EtOH/H₂O (300/100 mL, 3:1 ratio). The resulting mixture was sealed and heated at 80° C. Fe (12.97 g, 231.66 mmol) was then added at 80° C., sealed and heated at 80° C. for 1 h. The reaction mixture was cooled to room temperature, filtered, and diluted with EtOAc (300 mL×2). The solution was washed with water (300 mL×2), and the aqueous portion was extracted with EtOAc (300 mL×2). The combined organic extracts were washed with brine (300 mL×2), dried over anhydrous Na₂SO₄, filtrated, and concentrated in vacuo to afford the crude product. The crude product was purified by silica gel column chromatography (PE/EtOAc=10:1→3:1) to afford the 6-chloro-N²-(2,2-difluoroethyl)pyridine-2,3-diamine (L) (10.0 g, 48.167 mmol, 95.4% yield) as a purple solid. ¹H NMR (400 MHz, DMSO-d₆) δ 6.76 (d, J=7.8 Hz, 1H), 3.76-3.66 (m, 2H), 4.93 (s, 2H), 6.11 (tt, J=56.6, 4.2 Hz, 1H), 6.45 (d, J=7.8 Hz, 1H); ESIMS found for C₇H₈ClF₂N₃ m/z 208.1 (M+H).

### Step 3

To a solution of 6-chloro-N²-(2,2-difluoroethyl)pyridine-2,3-diamine (L) (10.0 g, 48.17 mmol) in HCl (360 mL) at 0° C. was added NaNO₂ (4.00 g, 57.97 mmol) in H₂O (55 mL). The mixture was stirred for room temperature at 16 h under N₂. The reaction mixture was adjusted to pH=9 with 4 M NaOH and extracted with EtOAc (300 mL×3). The combined organic layers were washed with brine and dried over anhydrous Na₂SO₄, filtered and concentrated to give the crude product. The crude product was purified by silica gel column chromatography (PE/EtOAc=10:1→1:1) to yield 5-chloro-3-(2,2-difluoroethyl)-3H-[1,2,3]triazolo[4,5-b]pyridine (LI) (6.2 g, 28.364 mmol, 58.9% yield) as a yellow solid. ¹H NMR (400 MHz, DMSO-d₆) δ 5.25 (td, J=16.0, 3.0 Hz, 2H), 6.85-6.41 (m, 1H), 7.65 (d, J=8.6 Hz, 1H), 8.72 (d, J=8.6 Hz, 1H); ESIMS found for C₇H₅ClF₂N₄ m/z 219.0 (M+H).

The following intermediates were prepared in accordance with the procedure described in the above Scheme 9.

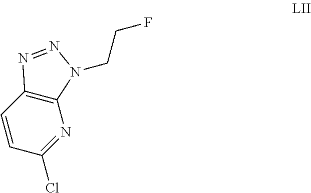 [SMILES: FCCn1nnc2ccc(Cl)nc21]

5-Chloro-3-(2-fluoroethyl)-3H-[1,2,3]triazolo[4,5-b]pyridine (LII): Yellow solid (2.7 g, 13.460 mmol, 39.3% yield). ¹H NMR (400 MHz, DMSO-d₆) δ 5.02-4.90 (m, 2H), 5.10-5.03 (m, 2H), 7.62 (d, J=8.6 Hz, 1H), 8.68 (d, J=8.6 Hz, 1H); ESIMS found for C₇H₆ClFN₄ m/z 201.1 (M+H).

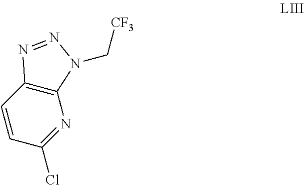 [SMILES: FC(F)(F)Cn1nnc2ccc(Cl)nc21]

5-Chloro-3-(2,2,2-trifluoroethyl)-3H-[1,2,3]triazolo[4,5-b]pyridine (LIII): Yellow solid (3.1 g, 13.103 mmol, 42.2%). ¹H NMR (400 MHz, DMSO-d₆) δ 5.83 (q, J=9.2 Hz, 2H), 7.70 (d, J=8.6 Hz, 1H), 8.76 (d, J=8.6 Hz, 1H); ESIMS found for C₇H₄ClF₃N₄ m/z 237.0 (M+H).

Preparation of intermediate cis-4-(methoxy-d₃)cyclohexan-1-amine (LIX) is depicted below in Scheme 10.

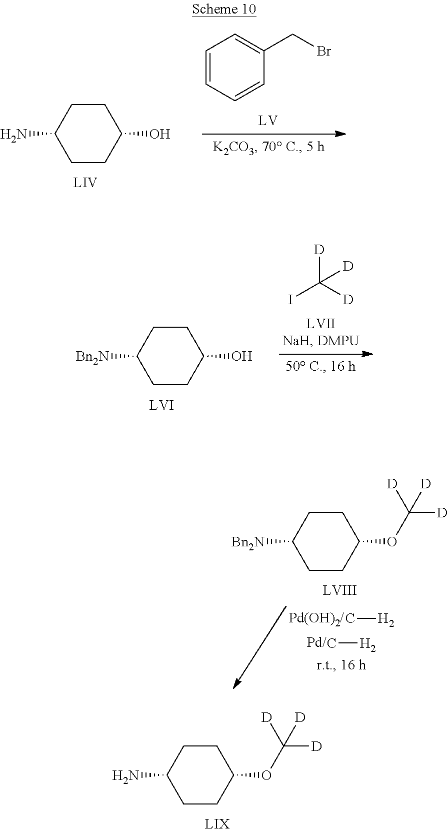

### Step 1

To a solution of cis-4-aminocyclohexan-1-ol (LIV) (5 g, 32.9 mmol), (bromomethyl)benzene (LV) (11.25 g, 65.8 mmol) in MeCN (80 mL) was added K₂CO₃ (13.64 g, 98.7 mmol). The mixture was stirred at 70° C. for 5 h. The reaction mixture was concentrated under reduced pressure to remove MeCN. The mixture was diluted with EtOAc and then extracted with EtOAc (100 mL×3) and H₂O. The combined organic layers were concentrated, and the crude residue was purified by silica gel column chromatography (0%→30% EtOAc/PE) to give the cis-4-(dibenzylamino)cyclohexan-1-ol (LVI) (8.0 g, 27.08 mmol, 82.3% yield) as a white solid. ESIMS found for C₂₀H₂₅NO m/z 296.4 (M+H).

### Step 2

To a solution of cis-4-(dibenzylamino)cyclohexan-1-ol (LVI) (8.0 g, 27.08 mmol) in DMPU (80 mL), NaH (5.98 g, 149.7 mmol) was added slowly under nitrogen atmosphere with continuous stirring. The reaction mixture was stirred at room temperature for 1 h. Then iodomethane-d₃ (LVII) (10.85 g, 74.86 mmol) was added at room temperature over a period of 10 min. After complete addition, the reaction mixture was stirred for 16 h at 50° C. The reaction mixture was then quenched with saturated aqueous NH₄Cl (300 mL) and stirred for 10 min. The mixture was diluted with EtOAc and then extracted with EtOAc (300 mL×3) and H₂O. The crude residue was purified by silica gel column chromatography (0%→20% EtOAc/PE) to yield cis-N,N-dibenzyl-4-(methoxy-d₃)cyclohexan-1-amine (LVIII) (6 g, 19.202 mmol, 70.9% yield) as a colorless oil. ESIMS found for C₂₁H₂₄D₃NO m/z 313.0 (M+H).

### Step 3

To a solution of cis-N,N-dibenzyl-4-(methoxy-d₃)cyclohexan-1-amine (LVIII) (200 mg, 0.64 mmol) in EtOH (5 mL) was added Pd(OH)₂/C (50 mg) and Pd/C (50 mg). The mixture was stirred at room temperature for 16 h. The mixture was filtered through Celite® and washed with EtOH. The reaction mixture was concentrated under reduced pressure to give the cis-4-(methoxy-d₃)cyclohexan-1-amine (LIX) (76.4 mg, 0.578 mmol, 90.3% yield) as a colorless oil. H NMR (400 MHz, DMSO-d₆) δ ppm 1.51-1.40 (m, 4H), 1.67-1.56 (m, 4H), 1.86 (td, J=9.8, 4.6 Hz, 2H), 2.71 (tt, J=10.8, 5.4 Hz, 1H), 3.34 (td, J=4.8, 2.4 Hz, 1H); ESIMS found for C₇H₁₂D₃NO m/z 133.0 (M+H).

The following intermediates were prepared in accordance with the procedure described in the above Scheme 10.

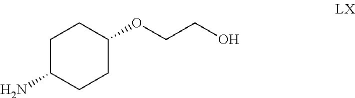 [SMILES: N[C@H]1CC[C@@H](OCCO)CC1]

2-((cis-4-Aminocyclohexyl)oxy)ethan-1-ol (LX): Colorless oil (0.5 g, 3.14 mmol, 67.4% yield). ESIMS found for C₈H₁₇NO₂ m/z 160. (M+H).

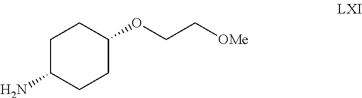 [SMILES: COCCO[C@H]1CC[C@@H](N)CC1]

cis-4-(2-Methoxyethoxy)cyclohexan-1-amine (LXI): Colorless oil (1.5 g, 8.65 mmol, 76.6% yield). ESIMS found for C₉H₁₉NO₂ m/z 174.1 (M+H).

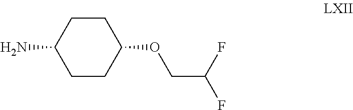 [SMILES: N[C@H]1CC[C@@H](OCC(F)F)CC1]

cis-4-(2,2-Difluoroethoxy)cyclohexan-1-amine (LXII): White solid (1.352 g, 7.54 mmol, 90.3% yield). ESIMS found for C₈H₁₅F₂NO m/z 180.1 (M+H).

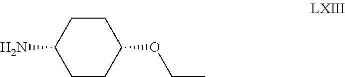

cis-4-Ethoxycyclohexan-1-amine (LXIII): Colorless oil (2 g, 13.96 mmol, 64.4% yield). ¹H NMR (400 MHz, CDCl₃) δ 1.19 (t, J=7.0 Hz, 3H), 1.49-1.44 (m, 6H), 1.62-1.55 (m, 2H), 1.86-1.79 (m, 2H), 2.4-2.723 (m, 1H), 3.49-3.41 (m, 3H).

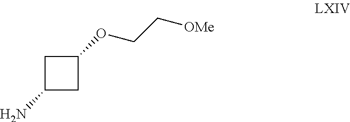

cis-3-(2-Methoxyethoxy)cyclobutan-1-amine (LXIV): Colorless oil. ¹H NMR (400 MHz, DMSO-d₆) δ 1.51 (dd, J=13.4, 5.2 Hz, 2H), 2.49-2.40 (m, 2H), 2.89-2.75 (m, 1H), 3.23 (s, 3H), 3.37-3.35 (m, 3H), 3.47 (s, 2H), 3.54-3.48 (m, 1H).

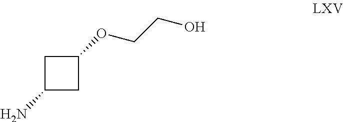

2-(cis-3-Aminocyclobutoxy)ethan-1-ol (LXV): Colorless oil. ¹H NMR (400 MHz, DMSO-d₆) δ 1.79-1.72 (m, 2H), 2.50-2.45 (m, 2H), 3.08-2.97 (m, 1H), 3.29 (t, J=5.4 Hz, 2H), 3.46 (t, J=5.4 Hz, 2H), 3.73-3.57 (m, 1H).

Preparation of intermediate cis-4-(difluoromethoxy)cyclohexan-1-amine (LXVIII) is depicted below in Scheme 11.

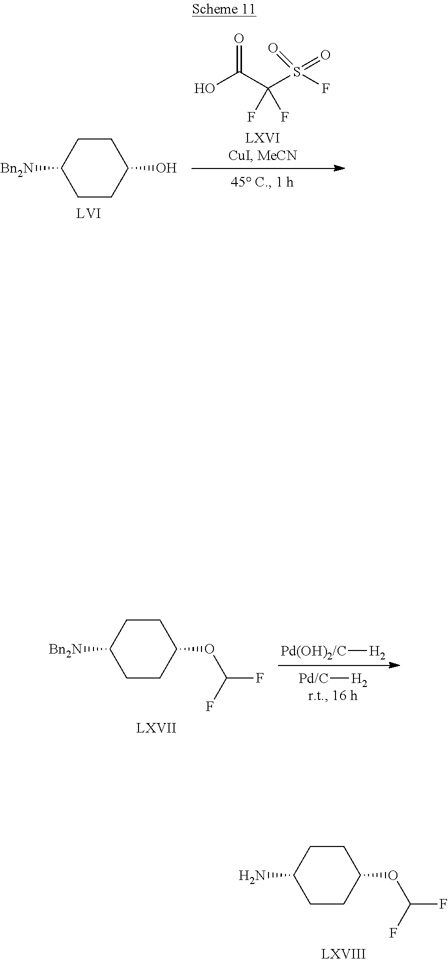

### Step 1

To a solution of cis-4-(dibenzylamino)cyclohexan-1-ol (LVI) (50 mg, 0.170 mmol), CuI (6.5 mg, 0.034 mmol) in MeCN (5 mL) and heated to 45° C. under nitrogen atmosphere for 5 min. To this mixture was added a solution of 2,2-difluoro-2-(fluorosulfonyl)acetic acid (LXVI) (60 mg, 0.339 mmol) in (2 mL) MeCN over 10 min. Then the mixture was stirred at 45° C. for 1 h. Volatile components were then removed via evaporation and the residue was diluted with EtOAc (100 mL) and 100 mL of a 1:1 mixture of water and saturated aqueous NaHCO₃. The resulting biphasic mixture containing solids was filtered through a sintered glass Buchner funnel. The filtrate layers were separated, and the aqueous layer was extracted with EtOAc (50 mL). The combined EtOAc layers were washed with 50 mL of a 1:1 mixture of brine and water, dried over anhydrous MgSO₄, filtered, and concentrated to an oil. The crude oil was purified by silica gel chromatography (100% hexanes→30% EtOAc/hexanes). Product containing fractions were combined and concentrated to afford the cis-N,N-dibenzyl-4-(difluoromethoxy)cyclohexan-1-amine (LXVII) (25 mg, 0.072 mmol, 42.3% yield) as an oil that solidified to an off-white solid. ESIMS found for C₂₁H₂₅F₂NO m/z 346.1 (M+H).

### Step 2

To a solution of cis-N,N-dibenzyl-4-(difluoromethoxy)cyclohexan-1-amine (LXVII) (2.8 g, 8.11 mmol) in THF (60 mL) was added Pd (OH)₂/C (1.4 g) and Pd/C (1.4 g). The mixture was stirred at room temperature for 16 h. The mixture was filtered through Celite® and washed with THF. The reaction mixture was concentrated under reduced pressure to afford cis-4-(difluoromethoxy)cyclohexan-1-amine (LXVIII) (1.05 g, 6.36 mmol, 78.4% yield) as a colorless oil. ESIMS found for C₇H₁₃F₂NO m/z 166.1 (M+H).

Preparation of intermediate 1-(3,3,3-trifluoropropyl)piperidin-4-amine (LXIII) is depicted below in Scheme 12.

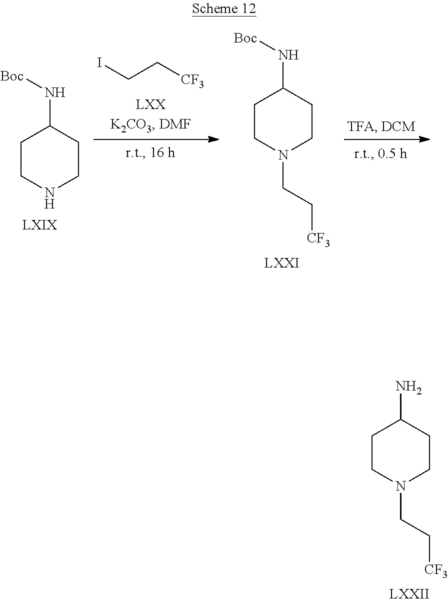 [SMILES: CC(C)(C)OC(=O)NC1CCNCC1 ; FC(F)(F)CCI ; CC(C)(C)OC(=O)NC1CCN(CCC(F)(F)F)CC1 ; NC1CCN(CCC(F)(F)F)CC1]

### Step 1

tert-Butyl piperidin-4-ylcarbamate (LXIX) (Commercially available from Combi-Blocks Inc.) (1 g, 4.99 mmol) and K₂CO₃ (1.73 g, 12.52 mmol) were dissolved in DMF (15 mL) and 1-iodo-3,3,3-trifluoropropane (LXX) (878 μL, 7.49 mmol) was added and the reaction was stirred at room temperature for 16 h. The reaction mixture was poured in EtOAc, and the aqueous layer was separated. The aqueous layer was extracted with EtOAc (×3) and then the combined organic layers were acidified to pH 4.5 with 1 M citric acid. The organic layer was washed three times with small volumes of water to remove unreacted SM. Sufficient amounts of the product remained in the organic layer which was dried using anhydrous MgSO₄ and reduced in vacuo to give the product tert-butyl N-[1-(3,3,3-trifluoropropyl)piperidin-4-yl]carbamate (LXXI) (861 mg, 2.906 mmol, 58.2% yield) as a white solid. ESIMS found for C₁₃H₂₃F₃N₂O₂ m/z 297.2 (M+H).

### Step 2

tert-Butyl N-[1-(3,3,3-trifluoropropyl)piperidin-4-yl]carbamate (LXXI) (200 mg, 0.670 mmol) was dissolved in DCE (3.2 mL) and TFA (800 μL, 10.38 mmol) was added and the reaction was stirred at room temperature for 30 m. The reaction mixture was blown dry and excess TFA removed by high vacuum to give the crude intermediate 1-(3,3,3-trifluoropropyl)piperidin-4-amine (LXXII) (209 mg, 0.674 mmol, 99.8% yield) as a white semi-solid which was used without further purification. ESIMS found for C₈H₁₅F₃N₂ m/z 197.1 (M+H).

Preparation of intermediate N-(trans-4-aminocyclohexyl)acetamide (LXXV) is depicted below in Scheme 13.

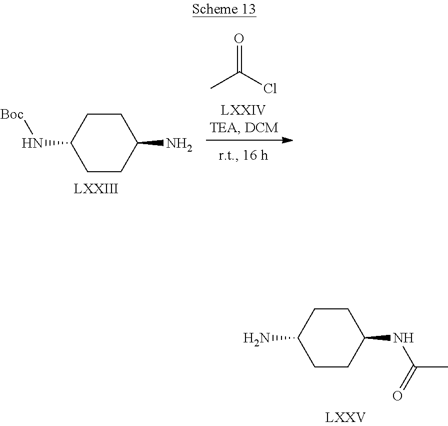

### Step 1

To a stirring solution of tert-butyl (trans-4-aminocyclohexyl)carbamate (LXXIII) (Commercially available from Combi-Blocks Inc.) (0.6 g, 2.8 mmol) in DCM (6 mL) was added TEA (1.2 mL, 8.61 mmol). Acetyl chloride (LXXIV) (0.22 mL, 3.09 mmol) was then slowly, and the reaction mixture was stirred at room temperature for 16 h. The solvent was removed, and the crude material was dissolved in EtOAc, washed with 1 M NaOH, brine, and dried over anhydrous MgSO₄ and finally concentrated. The product was dissolved in EtOH (2 mL) and 4 M HCl (1 mL). The solution was stirred at room temperature for 2 h before evaporating to dryness to give the HCl salt of N-(trans-4-aminocyclohexyl)acetamide (LXXV) (480 mg, 2.49 mmol, 89.0% yield) as a white solid. ESIMS found for C₈H₁₆N₂O m/z 157.05 (M+H).

Preparation of intermediate cis-4-amino-N,N-dimethylcyclohexane-1-carboxamide (LXXVII) is depicted below in Scheme 14.

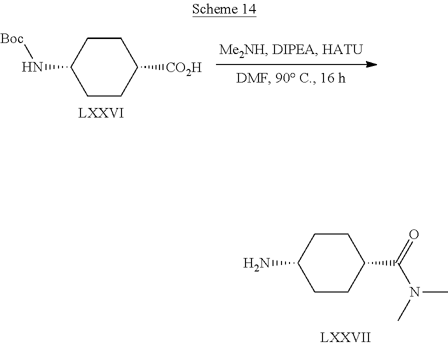 [SMILES: CC(C)(C)OC(=O)N[C@H]1CC[C@@H](C(=O)O)CC1 ; CN(C)C(=O)[C@H]1CC[C@@H](N)CC1]

### Step 1

To a stirring solution of 4-(tert-butoxycarbonylamino)cyclohexanecarboxylic acid (LXXVI) (Commercially available from Combi-Blocks Inc.) (0.3 g, 1.23 mmol) in DMF (6 mL) was added DIPEA (0.65 mL, 3.73 mmol) and HATU (0.7 g, 1.85 mmol). Reaction was stirred for 5 min at room temperature. Dimethylamine (0.92 mL, 1.84 mmol) was added, and reaction was heated to 90° C. for 16 h. The reaction was concentrated and dissolved in EtOH (2 mL) and 4 M HCl in dioxane (1 mL). The mixture was stirred for 2 h at room temperature, concentrated under vacuum to yield the HCl salt of 4-amino-N,N-dimethylcyclohexane-1-carboxamide (LXXVII) (280 mg, 1.355 mmol, 109.9% yield) as a light brown viscous solid. ESIMS found for C₉H₁₈N₂O m/z 171.15 (M+H).

The following intermediate was prepared in accordance with the procedure described in the above Scheme 14.

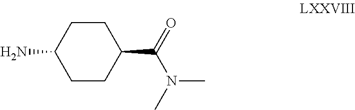

trans-4-Amino-N,N-dimethylcyclohexane-1-carboxamide (LXXVIII): Light brown viscous solid (290 mg, 1.403 mmol, 113.8% yield). ESIMS found for C₉H₁₈N₂O m/z 171.1 (M+H).

### Example 1

Preparation of 5-(3-(2,2-difluoroethyl)-2-methyl-3H-imidazo[4,5-b]pyridin-5-yl)-N²-((3S,4R)-3-fluoro-1-(oxetan-3-yl)piperidin-4-yl)-N⁴-methylpyrrolo[2,1-f][1,2,4]triazine-2,4-diamine (210) is depicted below in Scheme 15.

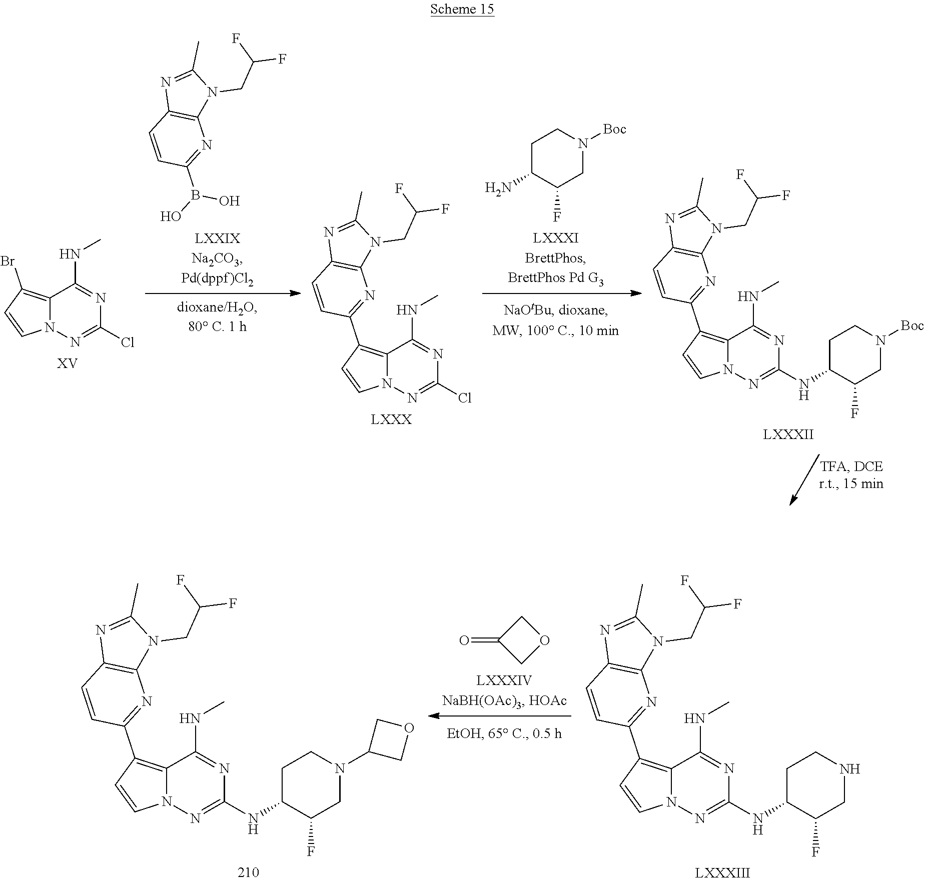

### Step 1

5-Bromo-2-chloro-N-methylpyrrolo[2,1-f][1,2,4]triazin-4-amine (XV) (1 g, 3.82 mmol), [3-(2,2-difluoroethyl)-2-methylimidazo[4,5-b]pyridin-5-yl]boronic acid (LXXIX) (1.01 g, 4.21 mmol), Pd(dppf)Cl2 (158 mg, 0.19 mmol) and NaHCO₃ (1.46 g, 13.77 mmol) were added to a mixture of dry 1,4-dioxane (20 mL) and water (5 mL). The reaction mixture was purged with Ar for 5 min, and the reaction was heated to 80° C. for 1 h. The reaction mixture was poured into aqueous saturated NH₄Cl solution and then extracted with DCM (×3), dried (MgSO₄) and reduced in vacuo to give a crude orange solid. The product was purified by column chromatography (0→100% EtOAc/hexanes followed by 0→6% MeOH/CHCl₃). The product was further purified by reverse phase column chromatography (10→70% MeCN/H₂O with 0.1% formic acid). Appropriate fractions were combined and neutralized with aqueous saturated NaHCO₃, extracted with DCM (×3), dried (MgSO₄) and reduced in vacuo to give a brown solid. The solid was triturated in MeOH and filtered while washing with MeOH. The product 2-chloro-5-[3-(2,2-difluoroethyl)-2-methylimidazo[4,5-b]pyridin-5-yl]-N-methylpyrrolo[2,1-f][1,2,4]triazin-4-amine (LXXI) (1.005 g, 2.660 mmol, 69.6% yield) was collected as an off-white solid. Impure fractions were combined and worked-up as previously described to give a second batch of product 2-chloro-5-[3-(2,2-difluoroethyl)-2-methylimidazo[4,5-b]pyridin-5-yl]-N-methylpyrrolo[2,1-f][1,2,4]triazin-4-amine (LXXX) (444 mg, 1.175 mmol, 30.7% yield) as an off-white solid. ESIMS found for C₁₆H₁₄ClF₂N₇ m/z 378.1 (M+H).

### Step 2

2-Chloro-5-[3-(2,2-difluoroethyl)-2-methylimidazo[4,5-b]pyridin-5-yl]-N-methylpyrrolo[2,1-f][1,2,4]triazin-4-amine (LXXX) (150 mg, 0.4 mmol), tert-butyl (3S,4R)-4-amino-3-fluoropiperidine-1-carboxylate (LXXXI) (commercially available from Advanced ChemBlocks Inc.) (104 mg, 0.48 mmol), BrettPhos Pd G₃ (30 mg, 0.03 mmol), BrettPhos (20 mg, 0.04 mmol), and NaO^{t}Bu (116 mg, 1.21 mmol) were added to dry 1,4-dioxane (3 mL) in a microwave vial. The suspension was purged with Ar for 5 min before the reaction was microwave irradiated to 100° C. for 10 min. The reaction mixture was reduced in vacuo and purified by column chromatography (0→100% EtOAc/hexanes followed by 0→3% 7.0 M NH₃ in MeOH/CHCl₃). The product was further purified by reverse phase column chromatography (10→70% MeCN/H₂O with 0.1% formic acid). Appropriate fractions were collected and neutralized with aqueous saturated NaHCO₃ and extracted with DCM (×2). The combined organic layers were dried (MgSO₄) and reduced in vacuo to give tert-butyl (3S,4R)-4-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-fluoropiperidine-1-carboxylate (LXXXII) (167 mg, 0.298 mmol, 75.2% yield) as a pale-yellow solid. ESIMS found for C₂₆H₃₂F₃N₉O₂ m/z 560.3 (M+H).

### Step 3

To a solution of tert-butyl (3S,4R)-4-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-fluoropiperidine-1-carboxylate (LXXXII) (167 mg, 0.3 mmol) in DCE (3 mL) was added TFA (1 mL, 12.98 mmol). The reaction was stirred at room temperature for 15 min. The reaction mixture turned an emerald-green color and the solvent was blown dry and excess TFA removed by high vacuum to give the crude intermediate (3S,4R)-4-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-fluoropiperidin-1-ium 2,2,2-trifluoroacetate (LXXXIII) (171 mg, 0.298 mmol, 99.9% yield), assuming quantitative yield, as a green semi-solid and was used without purification. ESIMS found for C₂₁H₂₄F₃N₉ m/z 460.25 (M+H).

### Step 4

5-(3-(2,2-Difluoroethyl)-2-methyl-3H-imidazo[4,5-b]pyridin-5-yl)-N2-((3S,4R)-3-fluoropiperidin-4-yl)-N4-methylpyrrolo[2,1-f][1,2,4]triazine-2,4-diamine (LXXXIII) (103 mg, 0.22 mmol) was dissolved in EtOH (2 mL) and HOAc (26 μL, 0.45 mmol) and oxetan-3-one (LXXXIV) (20 μL, 0.34 mmol) was added and the reaction was stirred for 10 m. NaBH(OAc)₃ (71 mg, 0.34 mmol) was added and the reaction was stirred at 65° C. for 30 min. The reaction mixture was loaded on Celite® and purified by column chromatography (0→2% hold 7.0 M NH₃ in MeOH/CHCl₃). Appropriate fractions were reduced in vacuo to give a white solid. The solid was triturated in MeOH and the solid filtered, washing with minimal MeOH to give 5-(3-(2,2-difluoroethyl)-2-methyl-3H-imidazo[4,5-b]pyridin-5-yl)-N²-((3S,4R)-3-fluoro-1-(oxetan-3-yl)piperidin-4-yl)-N⁴-methylpyrrolo[2,1-f][1,2,4]triazine-2,4-diamine (210) (64 mg, 0.124 mmol, 55.4% yield) as a white solid. ¹H NMR (499 MHz, DMSO-d₆) δ ppm 1.67-1.76 (1H, m), 1.81-1.91 (1H, m), 1.95-2.03 (1H, m), 2.06-2.20 (1H, m), 2.62 (3H, s), 2.75 (1H, br d, J=9.58 Hz), 2.92-3.02 (1H, m), 3.12 (3H, d, J=4.65 Hz), 3.48 (1H, quin, J=6.37 Hz), 3.72-3.89 (1H, m), 4.43 (2H, dt, J=29.30, 6.16 Hz), 4.54 (2H, td, J=6.50, 3.15 Hz), 4.72-4.83 (2H, m), 4.90 (1H, d, J=49.90 Hz), 5.95 (1H, d, J=8.21 Hz), 6.64 (1H, tt, J=54.30, 3.00 Hz), 7.10 (1H, d, J=3.01 Hz), 7.36 (1H, d, J=2.74 Hz), 7.85 (1H, d, J=8.49 Hz), 8.00 (1H, d, J=8.49 Hz), 10.97 (1H, q, J=4.38 Hz); ESIMS found for C₂₄H₂₈F₃N₉O m/z 516.3 (M+1).

### Example 2

Preparation of N²-((1R,5S,6r)-3-azabicyclo[3.1.0]hexan-6-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (219) and 1-((1R,5S,6r)-6-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-azabicyclo[3.1.0]hexan-3-yl)ethan-1-one (220) were depicted below in Scheme 16.

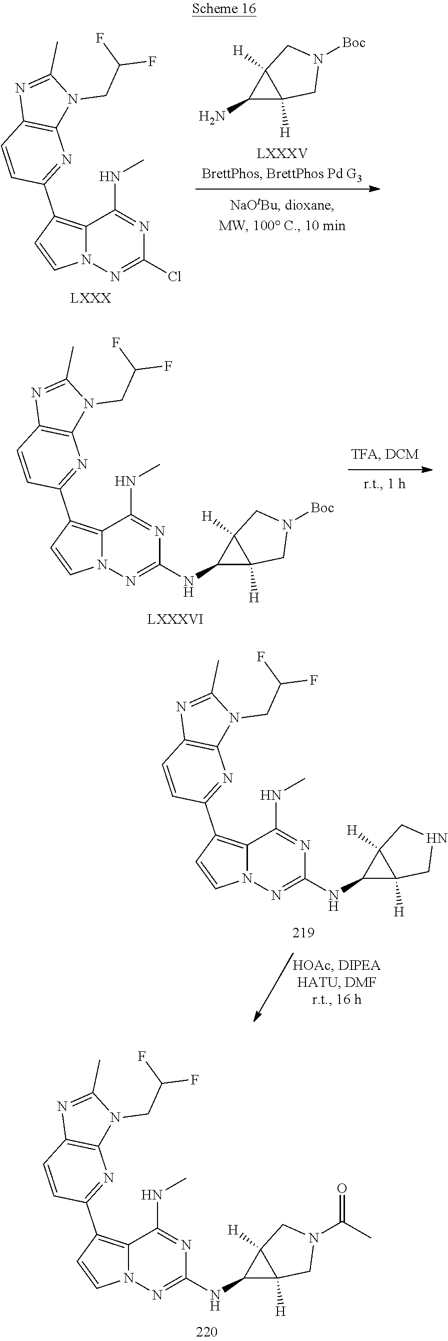 [SMILES: CNc1nc(Cl)nn2ccc(-c3ccc4nc(C)n(CC(F)F)c4n3)c12 ; CC(C)(C)OC(=O)N1C[C@@H]2[C@H](N)[C@@H]2C1 ; CNc1nc(N[C@@H]2[C@@H]3CN(C(=O)OC(C)(C)C)C[C@@H]32)nn2ccc(-c3ccc4nc(C)n(CC(F)F)c4n3)c12 ; CNc1nc(N[C@@H]2[C@@H]3CNC[C@@H]32)nn2ccc(-c3ccc4nc(C)n(CC(F)F)c4n3)c12 ; CNc1nc(N[C@@H]2[C@@H]3CN(C(C)=O)C[C@@H]32)nn2ccc(-c3ccc4nc(C)n(CC(F)F)c4n3)c12]

### Step 1

A mixture of 2-chloro-5-[3-(2,2-difluoroethyl)-2-methylimidazo[4,5-b]pyridin-5-yl]-N-methylpyrrolo[2,1-f][1,2,4]triazin-4-amine (LXXX) (100 mg, 0.26 mmol), tert-butyl (1R,5S,6r)-6-amino-3-azabicyclo[3.1.0]hexane-3-carboxylate (LXXXV) (commercially available from Combi-Blocks Inc.) (74 mg, 0.37 mmol), NaO^{t}Bu (70 mg, 0.73 mmol), BrettPhos Pd G₃ (20 mg, 0.02 mmol), and BrettPhos (14 mg, 0.03 mmol) in 1,4-dioxane (2 mL) was purged with N₂. The reaction was microwave irradiated to 100° C. for 10 min. The reaction mixture was filtered, concentrated and the crude purified by ISCO (0→10% MeOH/CHCl₃). Fractions were collected, concentrated under reduced pressure and the residue triturated in MTBE. The resulting solid was filtered and dried under high vacuo to obtain tert-butyl (1R,5S,6r)-6-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-azabicyclo[3.1.0]hexane-3-carboxylate (LXXXVI) (132 mg, 0.245 mmol, 92.4% yield) as an off-white solid. ESIMS found for C₂₆H₃₁F₂N₉O₂ m/z 540.3 (M+H).

### Step 2

To a stirred solution of tert-butyl (1R,5S,6r)-6-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-azabicyclo[3.1.0]hexane-3-carboxylate (LXXXVI) (130 mg, 0.24 mmol) in DCM (2 mL) was added TFA (1 mL, 12.98 mmol) and the mixture was stirred at room temperature for 1 h. The reaction mixture was concentrated, and the crude was purified by ISCO (1-10% 7 N NH₃ MeOH/CHCl₃). The pure fractions were collected, concentrated under reduced pressure and dried under high vacuo to obtain N²-((1R,5S,6r)-3-azabicyclo[3.1.0]hexan-6-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (219) (65 mg, 0.148 mmol, 61.4% yield) as an off-white solid. ¹H NMR (499 MHz, DMSO-d₆) δ ppm 1.64 (2H, br d, J=7.12 Hz), 2.62 (3H, s), 2.88-2.92 (1H, m), 2.92-2.97 (2H, m), 2.99-3.04 (2H, m), 3.11 (3H, d, J=4.65 Hz), 4.71-4.83 (2H, m), 6.04 (1H, d, J=4.11 Hz), 6.63 (1H, tt, J=54.30, 3.00 Hz), 7.10 (1H, d, J=2.74 Hz), 7.39 (1H, d, J=2.46 Hz), 7.85 (1H, d, J=8.49 Hz), 8.00 (1H, d, J=8.49 Hz), 10.93 (1H, br q, J=4.65 Hz); ESIMS found for C₂₁H₂₃F₂N₉ m/z 440.2 (M+1).

### Step 3

To a solution of HOAc (5.52 μL, 0.1 mmol), HATU (34 mg, 0.09 mmol) in DMF (200 μL) was added DIPEA (38 μL, 0.22 mmol). The mixture was stirred for 5 min. N²-((1R,5S,6r)-3-Azabicyclo[3.1.0]hexan-6-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (219) (30 mg, 0.07 mmol) in DMF (300 μL) was added and the reaction mixture was stirred at room temperature for 16 h. Water (10 mL) was added and the solution was extracted with EtOAc. The organics were separated, concentrated, absorbed on silica gel, and purified by preparative TLC (4% MeOH/CHCl₃) to give 1-((1R,5S,6r)-6-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)-3-azabicyclo[3.1.0] hexan-3-yl)ethan-1-one (220) (24 mg, 0.050 mmol, 73.0% yield) as an off-white solid. ¹H NMR (499 MHz, DMSO-d₆) δ ppm 1.83 (3H, s), 1.84-1.88 (1H, m), 1.89-1.95 (1H, m), 2.62 (3H, s), 2.69 (1H, td, J=6.91, 1.23 Hz), 3.12 (3H, d, J=4.65 Hz), 3.40-3.49 (2H, m), 3.54 (1H, d, J=10.40 Hz), 3.67 (1H, dd, J=10.40, 5.48 Hz), 4.72-4.83 (2H, m), 6.23 (1H, s), 6.63 (1H, tt, J=54.30, 3.00 Hz), 7.11 (1H, d, J=3.01 Hz), 7.40 (1H, d, J=2.74 Hz), 7.85 (1H, d, J=8.76 Hz), 8.01 (1H, d, J=8.49 Hz), 10.94 (1H, q, J=4.56 Hz); ESIMS found for C₂₃H₂₅F₂N₉O m/z 482.2 (M+1).

### Example 3

Preparation of N-(trans-4-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)cyclohexyl)acetamide (187) is depicted below in Scheme 17.

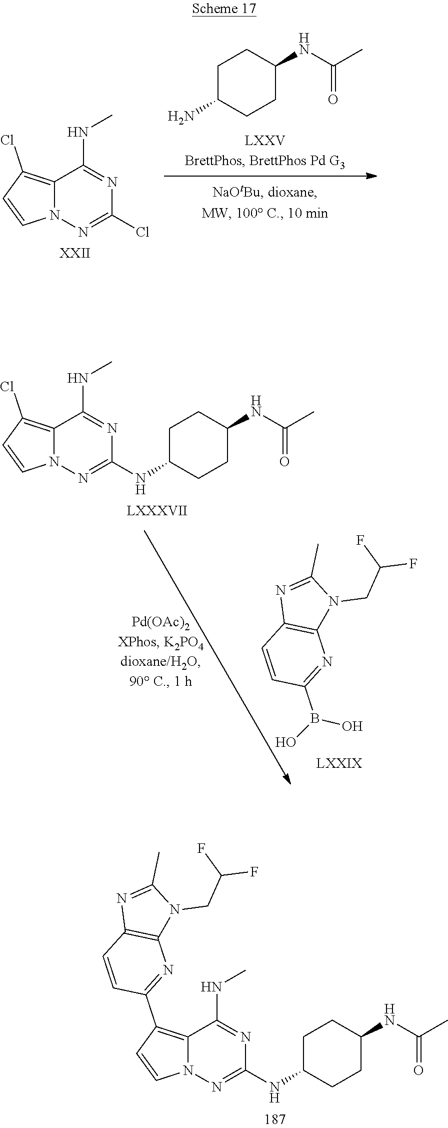

### Step 1

2,5-Dichloro-N-methylpyrrolo[2,1-f][1,2,4]triazin-4-amine (XXII) (100 mg, 0.46 mmol), NaO^{t}Bu (200 mg, 2.08 mmol), BrettPhos Pd G₃ (34. mg, 0.04 mmol), BrettPhos (25 mg, 0.05 mmol) and N-(trans-4-aminocyclohexyl)acetamide (LXXV) (124 mg, 0.64 mmol) dissolved in dry 1,4-dioxane (2 mL) were added to a microwave vessel and purged with Ar for 5 min. The reaction was microwave irradiated to 100° C. for 10 min. The reaction mixture was cooled and added to mixture of aqueous saturated NH₄Cl solution and DCM. The organic layer was separated, and the aqueous layer was extracted with DCM (×3), the combined organic layers were dried (MgSO₄) and reduced in vacuo to give an orange oil. The crude product was purified by column chromatography (0→% 7.0 M NH₃ in MeOH/CHCl₃) to produce N-(trans-4-((5-chloro-4-(methylamino)pyrrolo[2,1-f][1,2,4]triazin-2-yl)amino)cyclohexyl)acetamide (LXXXVII) (87 mg, 0.258 mmol, 56.1% yield) as a beige solid. ESIMS found for C₁₅H₂₁ClN₆O m/z 337.2 (M+H).

### Step 2

N-(trans-4-((5-chloro-4-(methylamino)pyrrolo[2,1-f][1,2,4]triazin-2-yl)amino) cyclohexyl)acetamide (LXXXVII) (87 mg, 0.26 mmol), [3-(2,2-difluoroethyl)-2-methylimidazo[4,5-b]pyridin-5-yl]boronic acid (LXXIX) (82 mg, 0.34 mmol), Pd(OAc)₂ (7 mg, 0.03 mmol) and XPhos (14 mg, 0.03 mmol) dissolved in dry 1,4-dioxane (1.5 mL) were added to a microwave vial. The reaction mixture was purged with Ar for 5 min. before adding an aqueous 2 M solution of K₂PO₄ (388 μL, 0.78 mmol) and further purged with Ar for 2 min. The reaction was heated to 90° C. for 1 h. The reaction mixture was reduced under vacuum and purified by reverse phase column chromatography (C18 column) (0→80% MeCN/H₂O with 0.1% formic acid). Corresponding fractions were lyophilized to give N-(trans-4-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino) cyclohexyl)acetamide (187) (22 mg, 0.044 mmol, 17.1% yield) as an off-white solid. ¹H NMR (499 MHz, DMSO-d₆) δ ppm 1.18-1.27 (2H, m), 1.27-1.37 (2H, m), 1.78 (3H, s), 1.79-1.83 (2H, m), 1.97 (2H, br d, J=10.68 Hz), 2.62 (3H, s), 3.10 (3H, d, J=4.65 Hz), 3.44-3.60 (2H, m), 4.72-4.83 (2H, m), 5.90 (1H, d, J=8.49 Hz), 6.63 (1H, tt, J=54.30, 3.00 Hz), 7.08 (1H, d, J=3.01 Hz), 7.37 (1H, d, J=3.01 Hz), 7.72 (1H, d, J=7.94 Hz), 7.84 (1H, d, J=8.76 Hz), 8.00 (1H, d, J=8.49 Hz), 10.89 (1H, q, J=4.65 Hz); ESIMS found for C₂₄H₂₉F₂N₉O m/z 498.3 (M+1).

### Example 4

Preparation of N²-((3S,4R)-1-Cyclopropyl-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (???) is depicted below in Scheme 18.

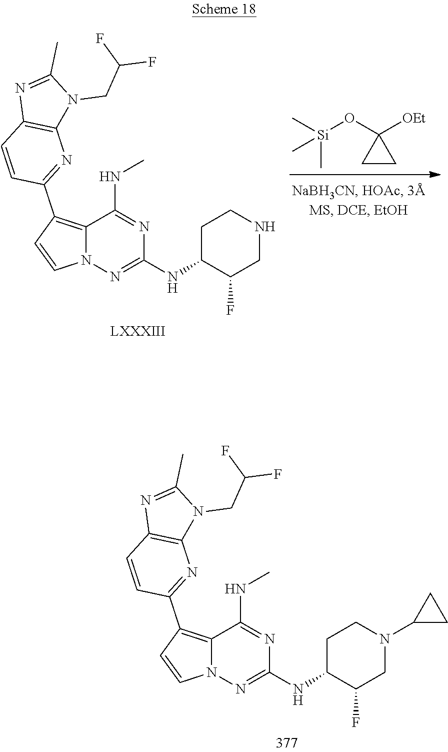 [SMILES: CNc1nc(N[C@@H]2CCNC[C@@H]2F)nn2ccc(-c3ccc4nc(C)n(CC(F)F)c4n3)c12 ; CCOC1(O[Si](C)(C)C)CC1 ; CNc1nc(N[C@@H]2CCN(C3CC3)C[C@@H]2F)nn2ccc(-c3ccc4nc(C)n(CC(F)F)c4n3)c12]

### Step 1

To a suspension of 5-(3-(2,2-difluoroethyl)-2-methyl-3H-imidazo[4,5-b]pyridin-5-yl)-N²-((3S,4R)-3-fluoropiperidin-4-yl)-N⁴-methylpyrrolo[2,1-f][1,2,4]triazine-2,4-diamine (LXXXIII) (30 mg, 0.06 mmol) and 2 beads of 3 AMS in dry DCE (0.9 mL) and ethanol (0.1 mL) was added HOAc (7 μL, 0.12 mmol) and (1-ethoxycyclopropoxy)trimethylsilane (commercially available from AmBeed, Inc.) (27 μL, 0.13 mmol). The reaction was stirred at 65° C. for 1 h. The reaction mixture was cooled to room temperature and NaBH₃CN (21 mg, 0.33 mmol) was added, and the reaction was stirred for 10 min. LCMS showed some starting material remained. Additional (1-ethoxycyclopropoxy)trimethylsilane (27 μL, 0.13 mmol) was added and the reaction was heated to 65° C. for 30 min. The reaction mixture was cooled and additional NaBH₃CN (21 mg, 0.33 mmol) was added, and the reaction was stirred for 10 min. LCMS confirmed the reaction to be complete. The reaction mixture was loaded onto Celite®, and the product was purified by column chromatography (0→4% MeOH/CHCl₃). The product was further purified by HPLC (0→40% MeCN/H₂O in 0.1% formic acid) to produce N²-((3S,4R)-1-Cyclopropyl-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (377) (14 mg, 0.028 mmol, 42.9% yield) as a pale-yellow fluffy solid after lyophilization. ¹H NMR (499 MHz, DMSO-d₆) δ ppm 0.20-0.29 (1H, m), 0.30-0.37 (1H, m), 0.39-0.49 (2H, m), 1.61-1.72 (2H, m), 1.79 (1H, qd, J=12.32, 3.83 Hz), 2.37 (1H, dd, J=45.80, 13.15 Hz), 2.33-2.38 (1H, m), 2.62 (3H, s), 2.95 (1H, br d, J=11.77 Hz), 3.12 (3H, d, J=4.65 Hz), 3.14-3.24 (1H, m), 3.70-3.88 (1H, m), 4.78 (2H, td, J=16.08, 2.60 Hz), 4.87 (1H, d, J=50.20 Hz), 5.87 (1H, br d, J=7.94 Hz), 6.64 (1H, tt, J=54.30, 3.00 Hz), 7.11 (1H, d, J=3.01 Hz), 7.37 (1H, d, J=2.74 Hz), 7.85 (1H, d, J=8.76 Hz), 8.01 (1H, d, J=8.49 Hz), 10.97 (1H, q, J=4.56 Hz); ESIMS found for C₂₄H₂₈F₃N₉ m/z 500.3 (M+1).

### Example 5

Preparation of N²-((3S,4R)-1-(3,3-Difluorocyclobutyl)-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (???) is depicted below in Scheme 19.

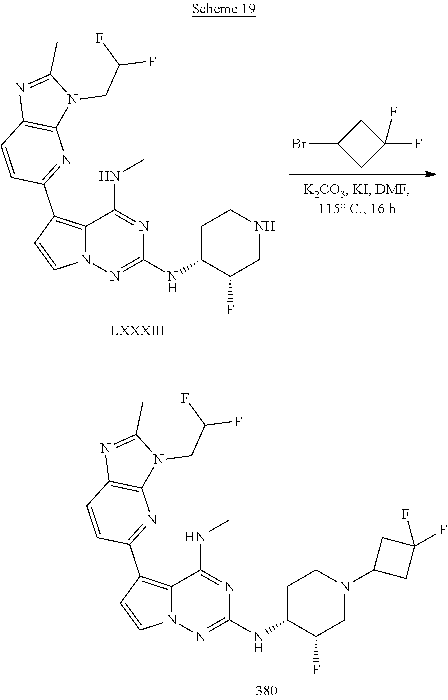 [SMILES: CNc1nc(N[C@@H]2CCNC[C@@H]2F)nn2ccc(-c3ccc4nc(C)n(CC(F)F)c4n3)c12 ; FC1(F)CC(Br)C1 ; CNc1nc(N[C@@H]2CCN(C3CC(F)(F)C3)C[C@@H]2F)nn2ccc(-c3ccc4nc(C)n(CC(F)F)c4n3)c12]

### Step 1

5-(3-(2,2-Difluoroethyl)-2-methyl-3H-imidazo[4,5-b]pyridin-5-yl)-N²-((3S,4R)-3-fluoropiperidin-4-yl)-N⁴-methylpyrrolo[2,1-f][1,2,4]triazine-2,4-diamine (LXXXIII) (65 mg, 0.14 mmol), K₂CO₃ (33 mg, 0.24 mmol) and KI (30 mg, 0.18 mmol) were suspended in DMF (1 mL). 3-Bromo-1,1-difluorocyclobutane (35 mg, 0.2 mmol) was added and stirred at 115° C. for 16 h. The reaction mixture was loaded onto Celite® and the product was purified by column chromatography (0→4% MeOH/CHCl₃). The product was further purified by HPLC (0→40% MeCN/H₂O in 0.1% formic acid) to give N²-((3S,4R)-1-(3,3-Difluorocyclobutyl)-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine (380) (6 mg, 0.011 mmol, 7.7% yield) as an off-white fluffy solid after lyophilization. ¹H NMR (499 MHz, DMSO-d₆) δ ppm 1.66-1.75 (1H, m), 1.85 (1H, qd, J=12.18, 3.70 Hz), 1.97-2.08 (1H, m), 2.16 (1H, dd, J=37.55, 12.32 Hz), 2.28-2.47 (2H, m), 2.62 (3H, s), 2.64-2.77 (3H, m), 2.83 (1H, br d, J=10.13 Hz), 3.02-3.09 (1H, m), 3.12 (3H, d, J=4.65 Hz), 3.73-3.89 (1H, m), 4.78 (2H, td, J=15.74, 2.19 Hz), 4.91 (1H, d, J=49.90 Hz), 5.95 (1H, d, J=8.21 Hz), 6.64 (1H, tt, J=54.30, 3.00 Hz), 7.11 (1H, d, J=3.01 Hz), 7.37 (1H, d, J=3.01 Hz), 7.85 (1H, d, J=8.76 Hz), 8.01 (1H, d, J=8.49 Hz), 10.98 (1H, q, J=4.56 Hz); ESIMS found for C₂₅H₂₈F₅N₉ m/z 550.3 (M+1).

### Example 6

Preparation of (1r,4r)-4-((4-Amino-5-(3-(2,2-difluoroethyl)-3H-imidazo[4,5-b]pyridin-5-yl)pyrrolo[2,1-f][1,2,4]triazin-2-yl)amino)-1-methylcyclohexan-1-ol (986) is depicted below in Scheme 20.

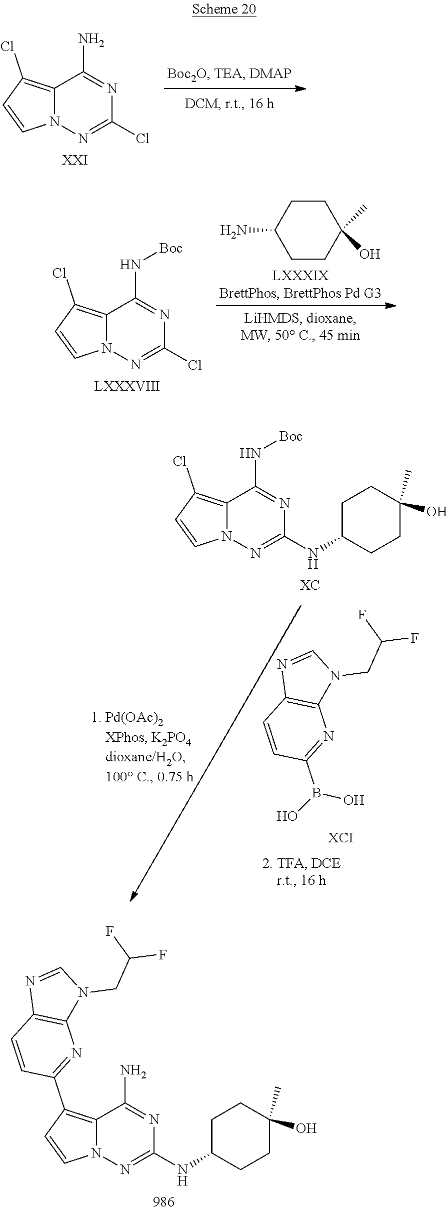

### Step 1

2,5-Dichloropyrrolo[2,1-f][1,2,4]triazin-4-amine (XXI) (100 mg, 0.49 mmol), di-tert-butyl decarbonate (162 mg, 0.74 mmol) and DMAP (8 mg, 0.07 mmol) were dissolved in DCM (4 mL). TEA (103 μL, 0.74 mmol) was added and the reaction was stirred at room temperature for 16 h. The reaction mixture was loaded onto Celite® and purified by silica gel column chromatography (EtOAc/hexanes=0→10) to produce tert-butyl (2,5-dichloropyrrolo[2,1-f][1,2,4]triazin-4-yl)carbamate (LXXXVIII) (107 mg, 0.353 mmol, 71.7% yield) as an off-white solid. ESIMS found for C₁₁H₁₂Cl₂N₄O₂ m/z 303.05 (M+H).

### Step 2

tert-Butyl (2,5-dichloropyrrolo[2,1-f][1,2,4]triazin-4-yl)carbamate (LXXXVIII) (200 mg, 0.66 mmol), LiHMDS (1.0 M in THF) (1.26 mL, 1.26 mmol), BrettPhos Pd G3 (30 mg, 0.03 mmol), BrettPhos (54 mg, 0.1 mmol) and trans-4-amino-1-methylcyclohexanol (LXXXIX) (94 mg, 0.73 mmol) were added to a microwave vial. Dry 1,4-dioxane (4 mL) was added and the suspension was purged with Ar for 5 minutes. The reaction was heated at 50° C. for 45 minutes. The reaction mixture was loaded onto Celite® and purified by silica gel column chromatography (EtOAc/hexanes=0→45%) to yield tert-butyl (5-chloro-2-(((1r,4r)-4-hydroxy-4-methylcyclohexyl)amino)pyrrolo[2,1-f][1,2,4]triazin-4-yl) carbamate (XC) (81 mg, 0.205 mmol, 31.0% yield) as an off-white solid. ESIMS found for C₁₈H₂₆ClN₅O₃ m/z 396.2 (M+1).

### Step 3

tert-Butyl (5-chloro-2-(((1r,4r)-4-hydroxy-4-methylcyclohexyl)amino) pyrrolo[2,1-f][1,2,4]triazin-4-yl) carbamate (XC) (27 mg, 0.07 mmol), [3-(2,2-difluoroethyl)imidazo[4,5-b]pyridin-5-yl]boronic acid (XCI) (22 mg, 0.1 mmol), Pd(OAc)₂ (1.6 mg, 0.01 mmol), and XPhos (5.1 mg, 0.01 mmol) were suspended in dry 1,4-dioxane (1 mL). An aqueous 2 M solution of K₂PO₄ (104.1 μL, 0.21 mmol) was then added and the reaction was sonicated and purged with Ar for 5 minutes. The reaction was heated to 100° C. for 45 minutes. To his mixture was added DCE (2 mL) followed by TFA (1 mL) and stirred at room temperature for 16 h. The reaction solvent was blown off with a stream of N₂, the residue was purified by silica gel column chromatography (MeOH/CHCl₃=0→10%), the solvent was removed from the fraction containing product and re-purified by C18 prep HPLC (MeCN/water (01% formic acid)=0→50%). The fractions containing product were frozen and lyophilized to give (1r,4r)-4-((4-Amino-5-(3-(2,2-difluoroethyl)-3H-imidazo[4,5-b]pyridin-5-yl)pyrrolo[2,1-f][1,2,4]triazin-2-yl)amino)-1-methylcyclohexan-1-ol (986) (8 mg, 0.018 mmol, 26.5% yield) as a white solid. ¹H NMR (499 MHz, DMSO-d₆) δ ppm 1.14 (3H, s), 1.34-1.46 (4H, m), 1.51-1.62 (2H, m), 1.77-1.89 (2H, m), 3.62 (1H, dt, J=8.08, 3.90 Hz), 4.20 (1H, br s), 4.74 (2H, td, J=16.50, 2.33 Hz), 5.77 (1H, d, J=8.21 Hz), 6.47 (1H, tt, J=54.45, 2.75 Hz), 7.11 (1H, d, J=3.01 Hz), 7.40 (1H, d, J=3.01 Hz), 7.45 (1H, br s), 7.87 (1H, d, J=8.76 Hz), 8.14 (1H, d, J=8.49 Hz), 8.39 (1H, s), 10.49 (1H, br s); ESIMS found for C₂₁H₂₄F₂N₈O m/z 443.2 (M+1).

The following compounds were prepared in accordance with the procedures described in the above Schemes 1-20.

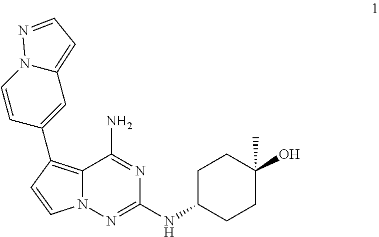 [SMILES: C[C@]1(O)CC[C@H](Nc2nc(N)c3c(-c4ccn5nccc5c4)ccn3n2)CC1]

### trans-4-((4-Amino-5-(pyrazolo[1,5-a]pyridin-5-yl)pyrrolo[2,1-f][1,2,4]triazin-2-yl)amino)-1-methylcyclohexan-1-ol 1

Beige solid (1.3 mg, 0.003 mmol, 1.0% yield). ¹H NMR (499 MHz, METHANOL-d₄) δ ppm 1.27 (3H, s), 1.44-1.55 (2H, m), 1.56-1.65 (2H, m), 1.67-1.76 (2H, m), 1.98-2.08 (2H, m), 3.15-3.23 (1H, m), 3.73 (1H, tt, J=8.73, 4.14 Hz), 4.60 (2H, br s), 6.57 (1H, d, J=2.46 Hz), 6.62 (1H, d, J=1.64 Hz), 7.02 (1H, dd, J=7.12, 1.92 Hz), 7.39 (1H, d, J=2.46 Hz), 7.68 (1H, d, J=1.09 Hz), 7.97 (1H, d, J=2.46 Hz), 8.57 (1H, d, J=7.39 Hz); ESIMS found for C₂₀H₂₃N₇O m/z 378.2 (M+1).

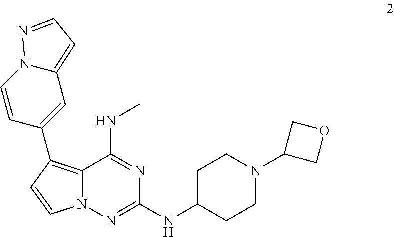 [SMILES: CNc1nc(NC2CCN(C3COC3)CC2)nn2ccc(-c3ccn4nccc4c3)c12]

### N⁴-Methyl-N²-(1-(oxetan-3-yl)piperidin-4-yl)-5-(pyrazolo[1,5-a]pyridin-5-yl)pyrrolo[2,1-f][1,2,4]triazine-2,4-diamine 2

Off-white solid (34 mg, 0.081 mmol, 50.7% yield). ¹H NMR (499 MHz, DMSO-d₆) δ ppm 1.46-1.57 (2H, m), 1.80-1.94 (4H, m), 2.67 (2H, br d, J=11.22 Hz), 2.86 (3H, d, J=4.65 Hz), 3.35-3.42 (1H, m), 3.52-3.63 (1H, m), 4.42 (2H, t, J=5.89 Hz), 4.53 (2H, t, J=6.43 Hz), 6.02 (1H, br d, J=8.21 Hz), 6.26 (1H, br q, J=4.65 Hz), 6.48 (1H, d, J=2.46 Hz), 6.59 (1H, d, J=1.64 Hz), 6.89 (1H, dd, J=7.39, 1.92 Hz), 7.39 (1H, d, J=2.74 Hz), 7.61 (1H, d, J=1.09 Hz), 7.99 (1H, d, J=2.19 Hz), 8.66 (1H, d, J=7.39 Hz); ESIMS found for C₂₂H₂₆N₈O m/z 419.3 (M+1).

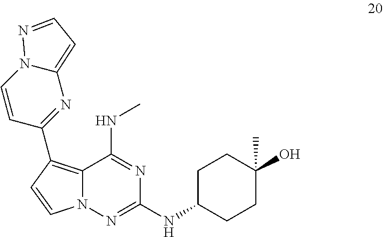 [SMILES: CNc1nc(N[C@H]2CC[C@](C)(O)CC2)nn2ccc(-c3ccn4nccc4n3)c12]

### trans-1-Methyl-4-((4-(methylamino)-5-(pyrazolo[1,5-a]pyrimidin-5-yl)pyrrolo[2,1-f][1,2,4]triazin-2-yl)amino)cyclohexan-1-ol 20

Beige solid (24 mg, 0.061 mmol, 12.6% yield). ¹H NMR (499 MHz, DMSO-d₆) δ ppm 1.14 (3H, s), 1.37-1.49 (4H, m), 1.54-1.64 (2H, m), 1.79-1.90 (2H, m), 3.10 (3H, d, J=4.65 Hz), 3.63 (1H, br s), 4.21 (1H, s), 6.05 (1H, d, J=7.94 Hz), 6.69 (1H, d, J=1.92 Hz), 7.30 (1H, d, J=3.01 Hz), 7.42 (1H, d, J=3.01 Hz), 7.54 (1H, d, J=7.67 Hz), 8.15 (1H, d, J=2.19 Hz), 9.01 (1H, d, J=7.67 Hz), 11.43 (1H, q, J=4.65 Hz); ESIMS found for C₂₀H₂₄N₈O m/z 393.25 (M+1).

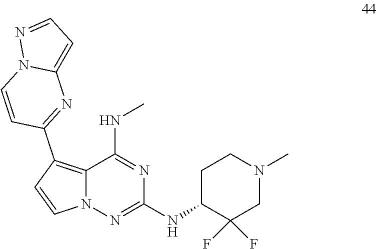 [SMILES: CNc1nc(N[C@@H]2CCN(C)CC2(F)F)nn2ccc(-c3ccn4nccc4n3)c12]

### (R)—N²-(3,3-Difluoro-1-methylpiperidin-4-yl)-N⁴-methyl-5-(pyrazolo[1,5-a]pyrimidin-5-yl)pyrrolo[2,1-f][1,2,4]triazine-2,4-diamine 44

Fluffy yellow solid (13 mg, 0.031 mmol, 83.7% yield). ¹H NMR (499 MHz, DMSO-d₆) δ ppm 1.73-1.88 (2H, m), 2.16 (1H, br s), 2.26 (3H, s), 2.31-2.45 (1H, m), 2.78 (1H, br d, J=10.40 Hz), 3.05 (1H, br s), 3.13 (3H, d, J=4.38 Hz), 4.16-4.32 (1H, m), 6.34 (1H, br d, J=9.31 Hz), 6.71 (1H, d, J=2.19 Hz), 7.34 (1H, d, J=3.01 Hz), 7.43 (1H, d, J=3.01 Hz), 7.56 (1H, d, J=7.67 Hz), 8.17 (1H, d, J=1.92 Hz), 9.03 (1H, d, J=7.67 Hz), 11.53 (1H, q, J=4.65 Hz); ESIMS found for C₁₉H₂₁F₂N₉ m/z 414.2 (M+1).

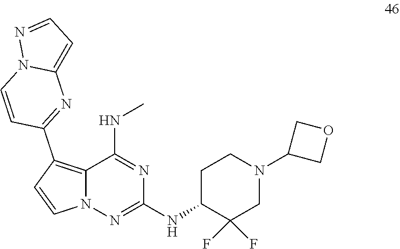 [SMILES: CNc1nc(N[C@@H]2CCN(C3COC3)CC2(F)F)nn2ccc(-c3ccn4nccc4n3)c12]

### (R)—N²-(3,3-Difluoro-1-(oxetan-3-yl)piperidin-4-yl)-N⁴-methyl-5-(pyrazolo[1,5-a]pyrimidin-5-yl)pyrrolo[2,1-f][1,2,4]triazine-2,4-diamine 46

Fluffy yellow solid (14 mg, 0.031 mmol, 64.6% yield). ¹H NMR (499 MHz, DMSO-d₆) δ ppm 1.74-1.83 (1H, m), 1.84-1.91 (1H, m), 2.14 (1H, br t, J=10.68 Hz), 2.37 (1H, ddd, J=27.15, 11.80, 1.35 Hz), 2.75 (1H, br d, J=11.23 Hz), 2.95-3.06 (1H, m), 3.13 (3H, d, J=4.65 Hz), 3.59 (1H, quin, J=6.30 Hz), 4.21-4.37 (1H, m), 4.44 (2H, dt, J=14.65, 6.09 Hz), 4.55 (2H, td, J=6.64, 3.42 Hz), 6.42 (1H, d, J=9.58 Hz), 6.72 (1H, d, J=2.19 Hz), 7.34 (1H, d, J=3.01 Hz), 7.43 (1H, d, J=3.01 Hz), 7.56 (1H, d, J=7.67 Hz), 8.17 (1H, d, J=2.19 Hz), 9.03 (1H, d, J=7.67 Hz), 11.54 (1H, q, J=4.56 Hz); ESIMS found for C₂₁H₂₃F₂N₉O m/z 456.2 (M+1).

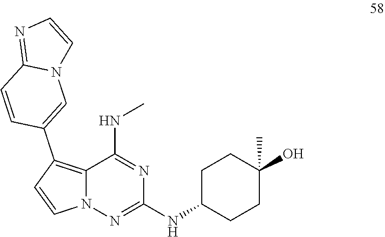 [SMILES: CNc1nc(N[C@H]2CC[C@](C)(O)CC2)nn2ccc(-c3ccc4nccn4c3)c12]

### trans-4-((5-(Imidazo[1,2-a]pyridin-6-yl)-4-(methylamino)pyrrolo[2,1-f][1,2,4]triazin-2-yl)amino)-1-methylcyclohexan-1-ol 58

Beige solid (47 mg, 0.120 mmol, 23.9% yield). ¹H NMR (499 MHz, DMSO-d₆) δ ppm 1.14 (3H, s), 1.36-1.48 (4H, m), 1.53-1.63 (2H, m), 1.79-1.90 (2H, m), 2.84 (3H, d, J=4.65 Hz), 3.54-3.66 (1H, m), 4.20 (1H, s), 5.83 (1H, d, J=8.21 Hz), 6.21 (1H, q, J=4.47 Hz), 6.41 (1H, d, J=2.46 Hz), 7.23 (1H, dd, J=9.31, 1.64 Hz), 7.39 (1H, d, J=2.46 Hz), 7.58 (1H, br d, J=9.03 Hz), 7.58 (1H, s), 7.94 (1H, s), 8.53 (1H, s) ESIMS found for C₂₁H₂₅N₇O m/z 392.3 (M+1).

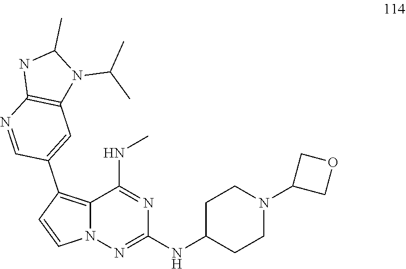 [SMILES: CNc1nc(NC2CCN(C3COC3)CC2)nn2ccc(-c3cnc4c(c3)N(C(C)C)C(C)N4)c12]

### 5-(1-(2,2-Difluoroethyl)-2-methyl-1H-imidazo[4,5-b]pyridin-6-yl)-N⁴-methyl-N²-(1-(oxetan-3-yl)piperidin-4-yl)pyrrolo[2,1-f][1,2,4]triazine-2,4-diamine 114

Off-white solid (8 mg, 0.017 mmol, 11.3% yield). ¹H NMR (499 MHz, DMSO-d₆) δ ppm 1.43-1.54 (2H, m), 1.57 (6H, d, J=6.84 Hz), 1.81-1.93 (3H, m), 1.96-2.03 (1H, m), 2.63 (3H, s), 2.67 (2H, br dd, J=6.57, 2.46 Hz), 2.82 (3H, d, J=4.65 Hz), 3.34-3.42 (1H, m), 3.53-3.64 (1H, m), 4.42 (2H, br s), 4.53 (2H, br s), 4.78 (1H, quin, J=6.98 Hz), 5.98 (1H, br d, J=7.67 Hz), 6.06 (1H, br d, J=1.37 Hz), 6.48 (1H, s), 7.40 (1H, d, J=2.46 Hz), 7.99 (1H, d, J=1.92 Hz), 8.37 (1H, d, J=1.92 Hz); ESIMS found for C₂₅H₃₃N₉O m/z 476.3 (M+1).

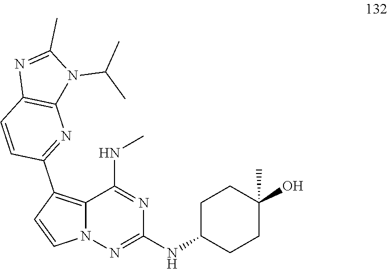 [SMILES: CNc1nc(N[C@H]2CC[C@](C)(O)CC2)nn2ccc(-c3ccc4nc(C)n(C(C)C)c4n3)c12]

### trans-4-((5-(3-Isopropyl-2-methyl-3H-imidazo[4,5-b]pyridin-5-yl)-4-(methylamino)pyrrolo[2,1-f][1,2,4]triazin-2-yl)amino)-1-methylcyclohexan-1-ol 132

Off-white solid (62 mg, 0.138 mmol, 73.4% yield). ¹H NMR (499 MHz, DMSO-d₆) δ ppm 1.14 (3H, s), 1.36-1.50 (4H, m), 1.54-1.62 (2H, m), 1.67 (6H, d, J=7.12 Hz), 1.81-1.90 (2H, m), 2.65 (3H, s), 3.12 (3H, d, J=4.65 Hz), 3.62 (1H, br s), 4.21 (1H, s), 4.92 (1H, dt, J=14.03, 7.08 Hz), 5.87 (1H, d, J=8.21 Hz), 7.01 (1H, d, J=3.01 Hz), 7.38 (1H, d, J=3.01 Hz), 7.75 (1H, d, J=8.49 Hz), 7.95 (1H, d, J=8.49 Hz), 10.50 (1H, br d, J=4.65 Hz); ESIMS found for C₂₄H₃₂N₈O m/z 449.3 (M+1).

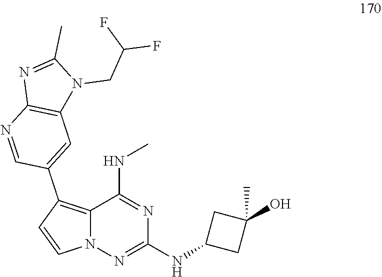 [SMILES: CNc1nc(N[C@H]2C[C@](C)(O)C2)nn2ccc(-c3cnc4nc(C)n(CC(F)F)c4c3)c12]

### trans-3-((5-(1-(2,2-Difluoroethyl)-2-methyl-1H-imidazo[4,5-b]pyridin-6-yl)-4-(methylamino)pyrrolo[2,1-f][1,2,4]triazin-2-yl)amino)-1-methylcyclobutan-1-ol 170

Off-white solid (13 mg, 0.029 mmol, 16.1% yield). ¹H NMR (499 MHz, DMSO-d₆) δ ppm 1.27 (3H, s), 1.92-2.00 (2H, m), 2.25-2.33 (2H, m), 2.63 (3H, s), 2.82 (3H, d, J=4.65 Hz), 4.25 (1H, sxt, J=7.45 Hz), 4.73 (1H, s), 4.77-4.90 (2H, m), 5.95 (1H, q, J=4.38 Hz), 6.32 (1H, d, J=7.12 Hz), 6.47 (1H, tt, J=54.60, 3.00 Hz), 6.44 (1H, d, J=2.46 Hz), 7.40 (1H, d, J=2.46 Hz), 7.98 (1H, d, J=1.92 Hz), 8.40 (1H, d, J=1.92 Hz); ESIMS found for C₂₁H₂₄F₂N₈O m/z 443.25 (M+1).

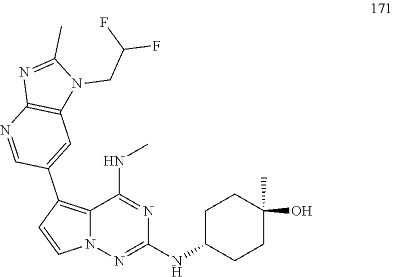 [SMILES: CNc1nc(N[C@H]2CC[C@](C)(O)CC2)nn2ccc(-c3cnc4nc(C)n(CC(F)F)c4c3)c12]

### trans-4-((5-(1-(2,2-Difluoroethyl)-2-methyl-1H-imidazo[4,5-b]pyridin-6-yl)-4-(methylamino)pyrrolo[2,1-f][1,2,4]triazin-2-yl)amino)-1-methylcyclohexan-1-ol 171

Beige solid (7 mg, 0.015 mmol, 8.1% yield). ¹H NMR (499 MHz, DMSO-d₆) δ ppm 1.14 (3H, s), 1.38-1.49 (4H, m), 1.54-1.62 (2H, m), 1.80-1.88 (2H, m), 2.63 (4H, s), 2.82 (3H, d, J=4.65 Hz), 3.62 (1H, br d, J=4.38 Hz), 4.20 (1H, s), 4.77-4.89 (2H, m), 5.83 (1H, d, J=8.21 Hz), 5.94 (1H, q, J=4.65 Hz), 6.47 (1H, tt, J=54.60, 3.00 Hz), 6.44 (1H, d, J=2.46 Hz), 7.42 (1H, d, J=2.46 Hz), 7.98 (1H, d, J=1.64 Hz), 8.40 (1H, d, J=2.19 Hz); ESIMS found for C₂₃H₂₈F₂N₈O m/z 471.3 (M+1).

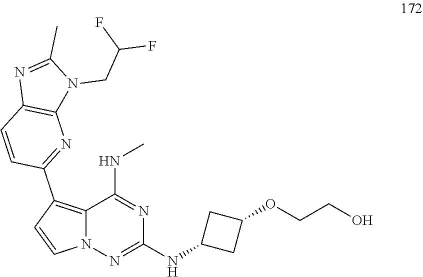 [SMILES: CNc1nc(N[C@H]2C[C@@H](OCCO)C2)nn2ccc(-c3ccc4nc(C)n(CC(F)F)c4n3)c12]

### 2-(cis-3-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)cyclobutoxy)ethan-1-ol 172

White solid (35 mg, 0.074 mmol, 56.0% yield). ¹H NMR (499 MHz, DMSO-d₆) δ ppm 1.84 (2H, qd, J=8.49, 2.74 Hz), 2.57-2.61 (2H, m), 2.62 (3H, s), 3.11 (3H, d, J=4.65 Hz), 3.29-3.33 (2H, m), 3.48 (2H, q, J=5.38 Hz), 3.69 (1H, quin, J=7.19 Hz), 3.78 (1H, dq, J=16.02, 8.08 Hz), 4.59 (1H, t, J=5.61 Hz), 4.72-4.82 (2H, m), 6.42 (1H, d, J=7.67 Hz), 6.63 (1H, tt, J=54.35, 3.00 Hz), 7.08 (1H, d, J=3.01 Hz), 7.34 (1H, d, J=2.74 Hz), 7.84 (1H, d, J=8.76 Hz), 8.00 (1H, d, J=8.49 Hz), 10.92 (1H, q, J=4.47 Hz); ESIMS found for C₂₂H₂₆F₂N₈O₂ m/z 473.2 (M+1).

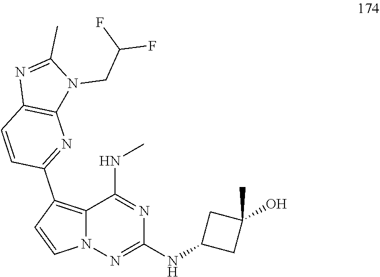 [SMILES: CNc1nc(N[C@H]2C[C@@](C)(O)C2)nn2ccc(-c3ccc4nc(C)n(CC(F)F)c4n3)c12]

### Cis-3-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-1-methylcyclobutan-1-ol 174

Beige solid (30 mg, 0.068 mmol, 51.2% yield). ¹H NMR (499 MHz, DMSO-d₆) δ ppm 1.25 (3H, s), 1.98 (2H, td, J=8.90, 2.19 Hz), 2.28-2.36 (2H, m), 2.62 (3H, s), 3.11 (3H, d, J=4.93 Hz), 3.72 (1H, sxt, J=7.78 Hz), 4.77 (2H, td, J=16.00, 2.20 Hz), 4.86 (1H, s), 6.28 (1H, d, J=6.84 Hz), 6.63 (1H, tt, J=54.60, 3.00 Hz), 7.08 (1H, d, J=3.01 Hz), 7.37 (1H, d, J=3.01 Hz), 7.84 (1H, d, J=8.76 Hz), 8.00 (1H, d, J=8.49 Hz), 10.90 (1H, q, J=4.56 Hz); ESIMS found for C₂₁H₂₄F₂N₈O m/z 443.2 (M+1).

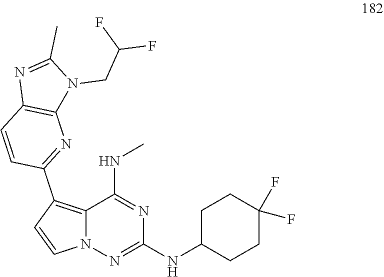 [SMILES: CNc1nc(NC2CCC(F)(F)CC2)nn2ccc(-c3ccc4nc(C)n(CC(F)F)c4n3)c12]

### N²-(4,4-Difluorocyclohexyl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 182

Off-white solid (15 mg, 0.032 mmol, 19.8% yield). ¹H NMR (499 MHz, DMSO-d₆) δ ppm 1.54-1.69 (2H, m), 1.83-2.01 (4H, m), 2.01-2.14 (2H, m), 2.62 (3H, s), 3.12 (3H, d, J=4.65 Hz), 3.70-3.84 (1H, m), 4.71-4.84 (2H, m), 6.15 (1H, d, J=7.67 Hz), 6.63 (1H, tt, J=54.30, 3.00 Hz), 7.09 (1H, d, J=3.01 Hz), 7.36 (1H, d, J=3.01 Hz), 7.84 (1H, d, J=8.49 Hz), 8.00 (1H, d, J=8.49 Hz), 10.92 (1H, q, J=4.56 Hz); ESIMS found for C₂₂H₂₄F₄N₈ m/z 477.2 (M+1).

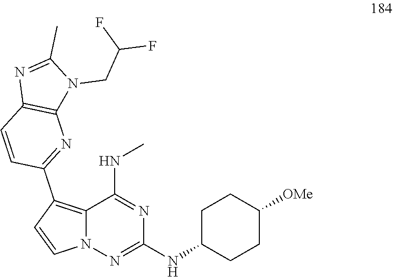 [SMILES: CNc1nc(N[C@H]2CC[C@@H](OC)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-(cis-4-methoxycyclohexyl)-N⁴-methylpyrrolo[2,1-f][1,2,4]triazine-2,4-diamine 183

White solid (6 mg, 0.013 mmol, 9.6% yield). ¹H NMR (499 MHz, DMSO-d₆) δ ppm 1.44-1.52 (2H, m), 1.53-1.62 (2H, m), 1.63-1.71 (2H, m), 1.79-1.88 (2H, m), 2.62 (3H, s), 3.11 (3H, d, J=4.65 Hz), 3.22 (3H, s), 3.29-3.32 (1H, m), 3.57-3.68 (1H, m), 4.71-4.83 (2H, m), 5.90 (1H, d, J=7.94 Hz), 6.63 (1H, tt, J=54.20, 3.00 Hz), 7.07 (1H, d, J=3.01 Hz), 7.35 (1H, d, J=2.74 Hz), 7.83 (1H, d, J=8.49 Hz), 7.99 (1H, d, J=8.49 Hz), 10.88 (1H, br q, J=4.65 Hz); ESIMS found for C₂₃H₂₈F₂N₈O m/z 471.3 (M+1).

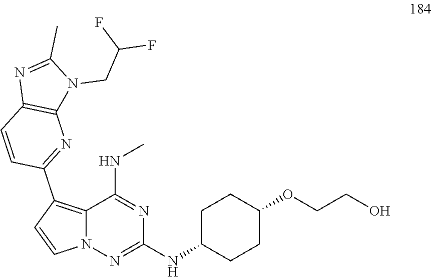 [SMILES: CNc1nc(N[C@H]2CC[C@@H](OCCO)CC2)nn2ccc(-c3ccc4nc(C)n(CC(F)F)c4n3)c12]

### 2-((cis-4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)cyclohexyl)oxy)ethan-1-ol 184

Off-white solid (16 mg, 0.03 mmol, 24.2% yield). ¹H NMR (499 MHz, DMSO-d₆) δ ppm 1.44-1.55 (2H, m), 1.58-1.73 (4H, m), 1.76-1.87 (2H, m), 2.62 (3H, s), 3.11 (3H, d, J=4.65 Hz), 3.39 (2H, t, J=5.50 Hz), 3.46 (1H, br s), 3.50 (2H, q, J=5.48 Hz), 3.56-3.67 (1H, m), 4.51 (1H, t, J=5.61 Hz), 4.70-4.82 (2H, m), 5.86 (1H, d, J=7.67 Hz), 6.63 (1H, tt, J=54.30, 2.75 Hz), 7.07 (1H, d, J=3.01 Hz), 7.35 (1H, d, J=2.74 Hz), 7.83 (1H, d, J=8.76 Hz), 7.99 (1H, d, J=8.49 Hz), 10.88 (1H, br q, J=4.65 Hz); ESIMS found for C₂₄H₃₀F₂N₈O₂ m/z 501.3 (M+1).

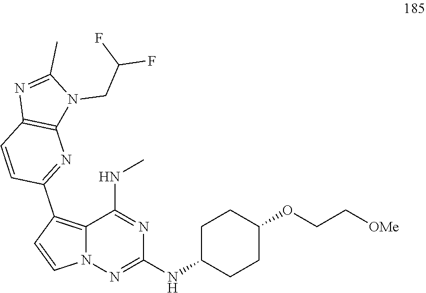 [SMILES: CNc1nc(N[C@H]2CC[C@@H](OCCOC)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-(cis-4-(2-methoxyethoxy)cyclohexyl)-N⁴-methylpyrrolo[2,1-f][1,2,4]triazine-2,4-diamine 185

White solid (29 mg, 0.056 mmol, 30.7% yield). ¹H NMR (499 MHz, DMSO-d₆) δ ppm 1.44-1.54 (2H, m), 1.57-1.72 (4H, m), 1.75-1.87 (2H, m), 2.62 (3H, s), 3.11 (3H, d, J=4.65 Hz), 3.27 (3H, s), 3.43-3.47 (3H, m), 3.47-3.52 (2H, m), 3.56-3.68 (1H, m), 4.71-4.84 (2H, m), 5.90 (1H, d, J=7.67 Hz), 6.63 (1H, tt, J=54.30, 3.00 Hz), 7.07 (1H, d, J=3.01 Hz), 7.35 (1H, d, J=2.74 Hz), 7.83 (1H, d, J=8.49 Hz), 7.99 (1H, d, J=8.49 Hz), 10.88 (1H, q, J=4.38 Hz); ESIMS found for C₂₅H₃₂F₂N₈O₂ m/z 515.3 (M+1).

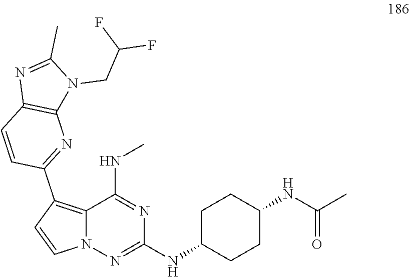 [SMILES: CNc1nc(N[C@H]2CC[C@@H](NC(C)=O)CC2)nn2ccc(-c3ccc4nc(C)n(CC(F)F)c4n3)c12]

### N-(cis-4-((5-(3-(2,2-difluoroethyl)-2-methyl-3H-imidazo[4,5-b]pyridin-5-yl)-4-(methylamino)pyrrolo[2,1-f][1,2,4]triazin-2-yl)amino)cyclohexyl)acetamide 186

Off-white solid (17 mg, 0.034 mmol, 74.1% yield). ¹H NMR (499 MHz, DMSO-d₆) δ ppm 1.49-1.58 (2H, m), 1.58-1.69 (4H, m), 1.71-1.79 (2H, m), 1.81 (3H, s), 2.62 (3H, s), 3.12 (3H, d, J=4.65 Hz), 3.29-3.32 (1H, m), 3.68 (2H, br s), 4.72-4.84 (2H, m), 5.72 (1H, d, J=6.57 Hz), 6.63 (1H, tt, J=54.30, 2.75 Hz), 7.08 (1H, d, J=3.01 Hz), 7.36 (1H, d, J=2.74 Hz), 7.68 (1H, br d, J=7.12 Hz), 7.84 (1H, d, J=8.76 Hz), 8.00 (1H, d, J=8.49 Hz), 10.91 (1H, q, J=4.38 Hz); ESIMS found for C₂₄H₂₉F₂N₉O m/z 498.3 (M+1).

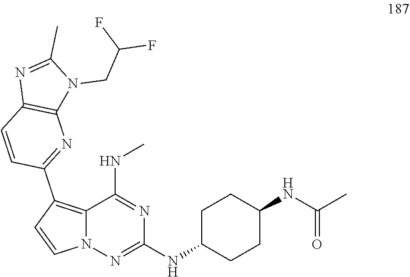 [SMILES: CNc1nc(N[C@H]2CC[C@H](NC(C)=O)CC2)nn2ccc(-c3ccc4nc(C)n(CC(F)F)c4n3)c12]

### N-(trans-4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)cyclohexyl)acetamide 187

Off-white solid (22 mg, 0.044 mmol, 17.1% yield). ¹H NMR (499 MHz, DMSO-d₆) δ ppm 1.18-1.27 (2H, m), 1.27-1.37 (2H, m), 1.78 (3H, s), 1.79-1.83 (2H, m), 1.97 (2H, br d, J=10.68 Hz), 2.62 (3H, s), 3.10 (3H, d, J=4.65 Hz), 3.44-3.60 (2H, m), 4.72-4.83 (2H, m), 5.90 (1H, d, J=8.49 Hz), 6.63 (1H, tt, J=54.30, 3.00 Hz), 7.08 (1H, d, J=3.01 Hz), 7.37 (1H, d, J=3.01 Hz), 7.72 (1H, d, J=7.94 Hz), 7.84 (1H, d, J=8.76 Hz), 8.00 (1H, d, J=8.49 Hz), 10.89 (1H, q, J=4.65 Hz); ESIMS found for C₂₄H₂₉F₂N₉O m/z 498.3 (M+1).

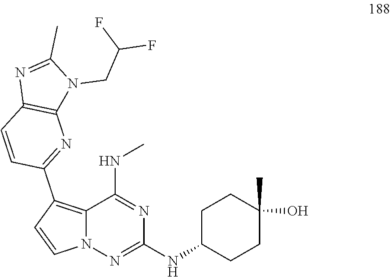 [SMILES: CNc1nc(N[C@H]2CC[C@@](C)(O)CC2)nn2ccc(-c3ccc4nc(C)n(CC(F)F)c4n3)c12]

### Cis-4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-1-methylcyclohexan-1-ol 188

Beige solid (27 mg, 0.057 mmol, 43.4% yield). ¹H NMR (499 MHz, DMSO-d₆) δ ppm 1.12 (3H, s), 1.35 (2H, td, J=13.14, 3.83 Hz), 1.57 (2H, br d, J=12.05 Hz), 1.59-1.66 (2H, m), 1.66-1.72 (2H, m), 2.62 (3H, s), 3.11 (3H, d, J=4.65 Hz), 3.45-3.56 (1H, m), 3.98 (1 H, s), 4.70-4.83 (2H, m), 5.81 (1H, d, J=7.94 Hz), 6.63 (1H, tt, J=54.60, 3.00 Hz), 7.07 (1H, d, J=3.01 Hz), 7.34 (1H, d, J=2.74 Hz), 7.83 (1H, d, J=8.76 Hz), 7.99 (1H, d, J=8.49 Hz), 10.87 (1H, q, J=4.38 Hz); ESIMS found for C₂₃H₂₈F₂N₈O m/z 471.3 (M+1).

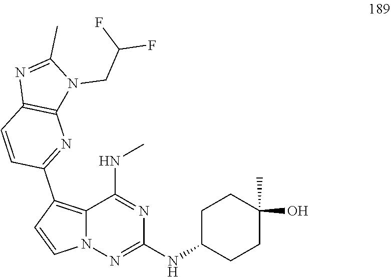 [SMILES: CNc1nc(N[C@H]2CC[C@](C)(O)CC2)nn2ccc(-c3ccc4nc(C)n(CC(F)F)c4n3)c12]

### trans-4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-1-methylcyclohexan-1-ol 189

Off-white solid (20 mg, 0.043 mmol, 26.3% yield). ¹H NMR (499 MHz, METHANOL-d₄) δ ppm 1.28 (3H, s), 1.46-1.56 (2H, m), 1.57-1.66 (2H, m), 1.69-1.77 (2H, m), 2.04 (2H, dq, J=13.18, 4.19 Hz), 2.68 (3H, s), 3.14-3.19 (3H, m), 3.70-3.80 (1H, m), 4.59 (1H, s), 4.75 (2H, td, J=15.26, 3.15 Hz), 5.49 (1H, s), 6.44 (1H, tt, J=54.60, 3.05 Hz), 7.00 (1H, d, J=3.01 Hz), 7.28 (1H, d, J=2.74 Hz), 7.83 (1H, d, J=8.76 Hz), 7.94 (1H, d, J=8.49 Hz), 10.87-10.94 (1H, m); ESIMS found for C₂₃H₂₈F₂N₈O m/z 471.3 (M+1).

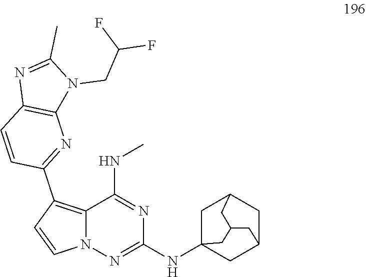 [SMILES: CNc1nc(NC23CC4CC(CC(C4)C2)C3)nn2ccc(-c3ccc4nc(C)n(CC(F)F)c4n3)c12]

### N²-(Adamantan-1-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 196

Off-white solid (25 mg, 0.062 mmol, 24.0% yield). ¹H NMR (499 MHz, DMSO-d₆) δ ppm 1.66 (6H, br s), 2.06 (3H, br s), 2.09 (6H, s), 2.62 (3H, s), 3.11 (3H, d, J=4.65 Hz), 4.77 (2H, td, J=15.95, 2.33 Hz), 5.36 (1H, s), 6.63 (1H, tt, J=54.30, 3.00 Hz), 7.08 (1H, d, J=2.74 Hz), 7.32 (1H, d, J=2.74 Hz), 7.83 (1H, d, J=8.49 Hz), 7.99 (1H, d, J=8.49 Hz), 10.84 (1H, q, J=4.29 Hz) ESIMS found for C₂₆H₃₀F₂N₈ m/z 493.3 (M+1).

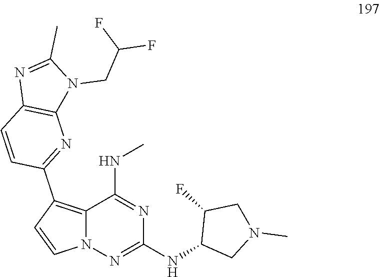 [SMILES: CNc1nc(N[C@H]2CN(C)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3S,4R)-4-fluoro-1-methylpyrrolidin-3-yl)-N⁴-methylpyrrolo[2,1-f][1,2,4]triazine-2,4-diamine 197

White solid (34 mg, 0.074 mmol, 43.4% yield). ¹H NMR (499 MHz, DMSO-d₆) δ ppm 2.31 (3H, s), 2.53-2.59 (2H, m), 2.62 (3H, s), 2.91 (1H, t, J=8.08 Hz), 3.12 (3H, d, J=4.65 Hz), 3.11-3.22 (1H, m), 4.22-4.36 (1H, m), 4.78 (2H, td, J=16.20, 2.75 Hz), 5.08-5.26 (1H, m), 6.05 (1H, d, J=8.21 Hz), 6.64 (1H, tt, J=54.60, 3.00 Hz), 7.12 (1H, d, J=3.01 Hz), 7.38 (1H, d, J=3.01 Hz), 7.85 (1H, d, J=8.49 Hz), 8.01 (1H, d, J=8.49 Hz), 10.99 (1H, q, J=4.38 Hz); ESIMS found for C₂₁H₂₄F₃N₉ m/z 460.25 (M+1).

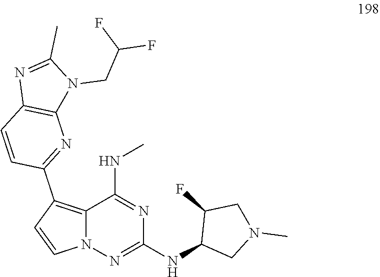 [SMILES: CNc1nc(N[C@@H]2CN(C)C[C@@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4S)-4-fluoro-1-methylpyrrolidin-3-yl)-N⁴-methylpyrrolo[2,1-f][1,2,4]triazine-2,4-diamine 198

White solid (41 mg, 0.089 mmol, 59.3% yield). ¹H NMR (499 MHz, DMSO-d₆) δ ppm 2.31 (3H, s), 2.56 (1H, br d, J=7.67 Hz), 2.62 (3H, s), 2.91 (1H, t, J=8.21 Hz), 3.09-3.22 (2H, m), 3.12 (3H, d, J=4.65 Hz), 4.22-4.36 (1H, m), 4.78 (2H, td, J=16.18, 2.75 Hz), 5.08-5.25 (1H, m), 6.05 (1H, d, J=8.21 Hz), 6.64 (1H, tt, J=54.30, 3.00 Hz), 7.12 (1H, d, J=3.01 Hz), 7.38 (1H, d, J=2.74 Hz), 7.85 (1H, d, J=8.49 Hz), 8.01 (1H, d, J=8.49 Hz), 10.99 (1H, q, J=4.38 Hz); ESIMS found for C₂₁H₂₄F₃N₉ m/z 460.2 (M+1).

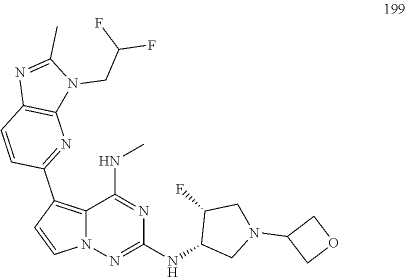 [SMILES: CNc1nc(N[C@H]2CN(C3COC3)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3S,4R)-4-fluoro-1-(oxetan-3-yl)pyrrolidin-3-yl)-N⁴-methylpyrrolo[2,1-f][1,2,4]triazine-2,4-diamine 199

Off-white solid (64 mg, 0.128 mmol, 41.5% yield). ¹H NMR (499 MHz, DMSO-d₆) δ ppm ¹H NMR (499 MHz, DMSO-d₆) δ ppm 2.59-2.66 (1H, m), 2.62 (3H, s), 2.74 (1H, ddd, J=30.45, 12.05, 1.37 Hz), 3.00 (1H, t, J=8.21 Hz), 3.09-3.24 (1H, m), 3.13 (3H, d, J=4.65 Hz), 3.75-3.82 (1H, m), 4.25-4.39 (1H, m), 4.46 (2H, t, J=6.02 Hz), 4.59 (2H, t, J=6.57 Hz), 4.78 (2H, td, J=15.95, 2.60 Hz), 5.20 (1H, dtd, J=55.95, 4.52, 4.52, 1.37 Hz), 6.12 (1H, d, J=7.94 Hz), 6.64 (1H, tt, J=54.30, 3.00 Hz), 7.12 (1H, d, J=3.01 Hz), 7.38 (1H, d, J=2.74 Hz), 7.86 (1H, d, J=8.49 Hz), 8.01 (1H, d, J=8.49 Hz), 11.00 (1H, q, J=4.56 Hz); ESIMS found for C₂₃H₂₆F₃N₉O m/z 502.3 (M+1).

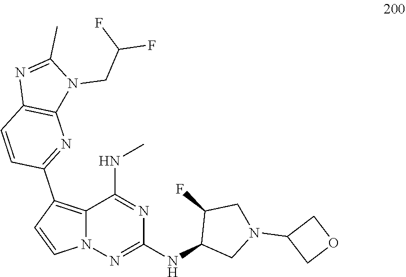 [SMILES: CNc1nc(N[C@@H]2CN(C3COC3)C[C@@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4S)-4-fluoro-1-(oxetan-3-yl)pyrrolidin-3-yl)-N⁴-methylpyrrolo[2,1-f][1,2,4]triazine-2,4-diamine 200

Off-white solid (72 mg, 0.144 mmol, 49.9% yield). ¹H NMR (499 MHz, DMSO-d₆) δ ppm 2.59-2.66 (1H, m), 2.62 (3H, s), 2.74 (1H, ddd, J=30.45, 12.05, 1.37 Hz), 3.00 (1H, t, J=8.21 Hz), 3.08-3.25 (1H, m), 3.13 (3H, d, J=4.65 Hz), 3.74-3.83 (1H, m), 4.24-4.38 (1H, m), 4.46 (2H, t, J=5.89 Hz), 4.59 (2H, t, J=6.57 Hz), 4.78 (2H, td, J=15.88, 2.46 Hz), 5.20 (1H, dtd, J=55.95, 4.52, 4.52, 1.37 Hz), 6.12 (1H, d, J=7.94 Hz), 6.64 (1H, tt, J=54.30, 3.00 Hz), 7.12 (1H, d, J=3.01 Hz), 7.38 (1H, d, J=2.74 Hz), 7.86 (1H, d, J=8.76 Hz), 8.01 (1H, d, J=8.49 Hz), 11.00 (1H, q, J=4.47 Hz); ESIMS found for C₂₃H₂₆F₃N₉O m/z 502.3 (M+1).

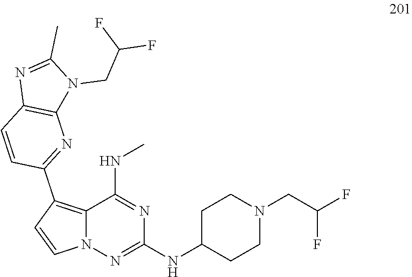 [SMILES: CNc1nc(NC2CCN(CC(F)F)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-(1-(2,2-difluoroethyl)piperidin-4-yl)-N⁴-methylpyrrolo[2,1-f][1,2,4]triazine-2,4-diamine 201

White solid (43 mg, 0.085 mmol, 53.6% yield). ¹H NMR (499 MHz, DMSO-d₆) δ ppm 1.45-1.57 (2H, m), 1.87 (2H, br d, J=10.13 Hz), 2.20-2.28 (2H, m), 2.62 (3H, s), 2.71 (2H, td, J=15.74, 4.38 Hz), 2.89 (2H, br d, J=11.77 Hz), 3.11 (3H, d, J=4.65 Hz), 3.51-3.62 (1H, m), 4.72-4.83 (2H, m), 5.98 (1H, d, J=8.21 Hz), 6.13 (1H, tt, J=55.95, 4.35 Hz), 6.63 (1H, tt, J=54.60, 3.00 Hz), 7.08 (1H, d, J=3.01 Hz), 7.36 (1H, d, J=2.74 Hz), 7.84 (1H, d, J=8.76 Hz), 8.00 (1H, d, J=8.49 Hz), 10.91 (1H, q, J=4.65 Hz); ESIMS found for C₂₃H₂₇F₄N₉ m/z 506.3 (M+1).

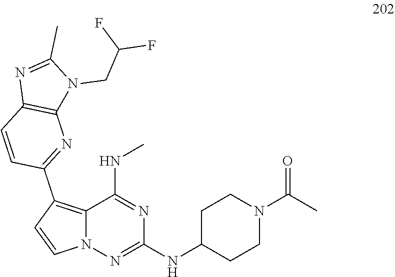 [SMILES: CNc1nc(NC2CCN(C(C)=O)CC2)nn2ccc(-c3ccc4nc(C)n(CC(F)F)c4n3)c12]

### 1-(4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)piperidin-1-yl)ethan-1-one 202

Beige solid (20 mg, 0.041 mmol, 15.6% yield). ¹H NMR (499 MHz, DMSO-d₆) δ ppm 1.78-1.86 (1H, m), 1.87-1.97 (1H, m), 2.35-2.42 (1H, m), 2.42-2.48 (1H, m), 2.50 (3H, s), 3.12 (3H, s), 3.17-3.27 (1H, m), 3.61 (3H, d, J=4.93 Hz), 4.11 (1H, dtd, J=13.21, 6.54, 6.54, 3.83 Hz), 4.26-4.34 (2H, m), 4.77 (1H, br d, J=14.24 Hz), 5.27 (2H, td, J=15.88, 2.46 Hz), 6.60 (1H, d, J=8.21 Hz), 7.13 (1H, tt, J=54.30, 3.00 Hz), 7.59 (1H, d, J=2.74 Hz), 7.87 (1H, d, J=3.01 Hz), 8.34 (1H, d, J=8.76 Hz), 8.50 (1H, d, J=8.49 Hz), 11.42 (1H, q, J=4.29 Hz); ESIMS found for C₂₃H₂₇F₂N₉O m/z 484.3 (M+1).

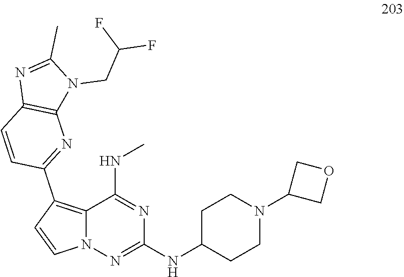 [SMILES: CNc1nc(NC2CCN(C3COC3)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-(1-(oxetan-3-yl)piperidin-4-yl)pyrrolo[2,1-f][1,2,4]triazine-2,4-diamine 203

Off-white solid (51 mg, 0.103 mmol, 49.3% yield). ¹H NMR (499 MHz, DMSO-d₆) δ ppm 1.45-1.57 (2H, m), 1.81-1.87 (2H, m), 1.89 (2H, br d, J=11.23 Hz), 2.62 (3H, s), 2.67 (2H, br d, J=10.95 Hz), 3.11 (3H, d, J=4.65 Hz), 3.35-3.41 (1H, m), 3.52-3.64 (1H, m), 4.42 (2H, t, J=6.02 Hz), 4.52 (2H, t, J=6.43 Hz), 4.71-4.83 (2H, m), 6.00 (1H, d, J=8.21 Hz), 6.63 (1H, tt, J=54.45, 3.00 Hz), 7.08 (1H, d, J=3.01 Hz), 7.36 (1H, d, J=2.74 Hz), 7.84 (1H, d, J=8.76 Hz), 8.00 (1H, d, J=8.49 Hz), 10.91 (1H, br q, J=4.70 Hz); ESIMS found for C₂₄H₂₉F₂N₉O m/z 498.3 (M+1).

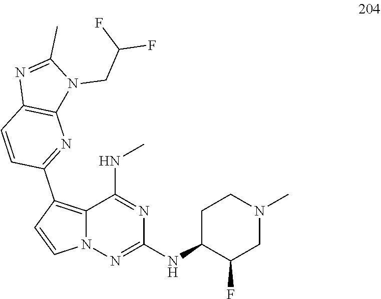 [SMILES: CNc1nc(N[C@H]2CCN(C)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4S)-3-fluoro-1-methylpiperidin-4-yl)-N⁴-methylpyrrolo[2,1-f][1,2,4]triazine-2,4-diamine 204

White solid (35 mg, 0.074 mmol, 38.5% yield). ¹H NMR (499 MHz, DMSO-d₆) δ ppm 1.68 (1H, br dd, J=12.59, 3.01 Hz), 1.87 (1H, qd, J=12.23, 3.56 Hz), 2.00-2.07 (1H, m), 2.10-2.22 (1H, m), 2.18 (3H, s), 2.79 (1H, br d, J=10.95 Hz), 3.04 (1H, br t, J=10.95 Hz), 3.12 (3H, d, J=4.65 Hz), 3.66-3.82 (1H, m), 4.78 (2H, td, J=15.90, 2.20 Hz), 4.88 (1H, d, J=49.90 Hz), 5.87 (1H, d, J=7.94 Hz), 6.64 (1H, tt, J=54.60, 2.75 Hz), 7.10 (1H, d, J=3.01 Hz), 7.37 (1H, d, J=2.74 Hz), 7.85 (1H, d, J=8.49 Hz), 8.00 (1H, d, J=8.49 Hz), 10.97 (1H, q, J=4.65 Hz); ESIMS found for C₂₂H₂₆F₃N₉ m/z 474.25 (M+1).

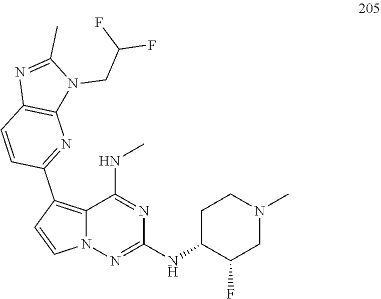 [SMILES: CNc1nc(N[C@@H]2CCN(C)C[C@@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3S,4R)-3-fluoro-1-methylpiperidin-4-yl)-N⁴-methylpyrrolo[2,1-f][1,2,4]triazine-2,4-diamine 205

White solid (40 mg, 0.085 mmol, 44.0% yield). ¹H NMR (499 MHz, DMSO-d₆) δ ppm 1.64-1.72 (1H, m), 1.87 (1H, qd, J=12.23, 3.83 Hz), 2.04 (1H, br t, J=10.95 Hz), 2.10-2.22 (1H, m), 2.18 (3H, s), 2.62 (3H, s), 2.79 (1H, br d, J=10.40 Hz), 3.04 (1H, br t, J=11.23 Hz), 3.12 (3H, d, J=4.65 Hz), 3.66-3.81 (1H, m), 4.73-4.81 (2H, m), 4.88 (1H, d, J=49.65 Hz), 5.87 (1H, d, J=8.21 Hz), 6.64 (1H, tt, J=54.30, 2.75 Hz), 7.10 (1H, d, J=3.01 Hz), 7.37 (1H, d, J=2.74 Hz), 7.85 (1H, d, J=8.49 Hz), 8.00 (1H, d, J=8.76 Hz), 10.97 (1H, q, J=4.65 Hz); ESIMS found for C₂₂H₂₆F₃N₉ m/z 474.3 (M+1).

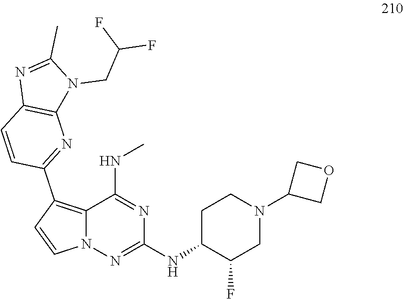 [SMILES: CNc1nc(N[C@@H]2CCN(C3COC3)C[C@@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3S,4R)-3-fluoro-1-(oxetan-3-yl)piperidin-4-yl)-N⁴-methylpyrrolo[2,1-f][1,2,4]triazine-2,4-diamine 210

White solid (64 mg, 0.124 mmol, 55.4% yield). ¹H NMR (499 MHz, DMSO-d₆) δ ppm 1.67-1.76 (1H, m), 1.81-1.91 (1H, m), 1.95-2.03 (1H, m), 2.06-2.20 (1H, m), 2.62 (3H, s), 2.75 (1H, br d, J=9.58 Hz), 2.92-3.02 (1H, m), 3.12 (3H, d, J=4.65 Hz), 3.48 (1H, quin, J=6.37 Hz), 3.72-3.89 (1H, m), 4.43 (2H, dt, J=29.30, 6.16 Hz), 4.54 (2H, td, J=6.50, 3.15 Hz), 4.72-4.83 (2H, m), 4.90 (1H, d, J=49.90 Hz), 5.95 (1H, d, J=8.21 Hz), 6.64 (1H, tt, J=54.30, 3.00 Hz), 7.10 (1H, d, J=3.01 Hz), 7.36 (1H, d, J=2.74 Hz), 7.85 (1H, d, J=8.49 Hz), 8.00 (1H, d, J=8.49 Hz), 10.97 (1H, q, J=4.38 Hz); ESIMS found for C₂₄H₂₈F₃N₉O m/z 516.3 (M+1).

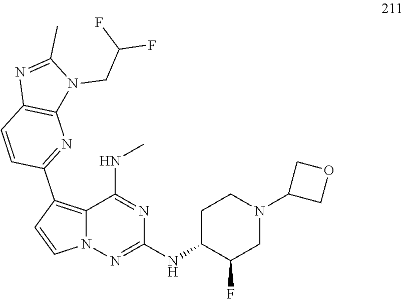 [SMILES: CNc1nc(N[C@@H]2CCN(C3COC3)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4R)-3-fluoro-1-(oxetan-3-yl)piperidin-4-yl)-N⁴-methylpyrrolo[2,1-f][1,2,4]triazine-2,4-diamine 211

Pale yellow solid (22 mg, 0.043 mmol, 65.4% yield). ¹H NMR (499 MHz, DMSO-d₆) δ ppm 1.44-1.57 (1H, m), 1.94 (1H, td, J=11.57, 2.05 Hz), 2.00 (2H, td, J=9.65, 5.34 Hz), 2.62 (3H, s), 2.62-2.67 (1H, m), 2.98-3.06 (1H, m), 3.12 (3H, d, J=4.65 Hz), 3.51 (1H, quin, J=6.30 Hz), 3.78-3.91 (1H, m), 4.42 (1H, t, J=6.16 Hz), 4.45 (1H, t, J=6.16 Hz), 4.50-4.66 (1H, m), 4.54 (2H, t, J=6.60 Hz), 4.78 (2H, td, J=16.20, 2.75 Hz), 6.32 (1H, d, J=8.76 Hz), 6.64 (1H, tt, J=54.30, 3.00 Hz), 7.09 (1H, d, J=3.01 Hz), 7.37 (1H, d, J=2.74 Hz), 7.85 (1H, d, J=8.76 Hz), 8.00 (1H, d, J=8.49 Hz), 10.96 (1H, q, J=4.56 Hz); ESIMS found for C₂₄H₂₈F₃N₉O m/z 516.3 (M+1).

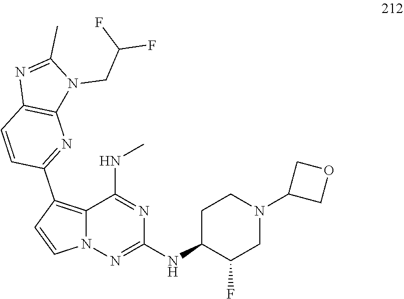 [SMILES: CNc1nc(N[C@H]2CCN(C3COC3)C[C@@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3S,4S)-3-fluoro-1-(oxetan-3-yl)piperidin-4-yl)-N⁴-methylpyrrolo[2,1-f][1,2,4]triazine-2,4-diamine 212

Pale yellow solid (23 mg, 0.045 mmol, 66.1% yield). ¹H NMR (499 MHz, DMSO-d₆) δ ppm 1.45-1.57 (1H, m), 1.91-1.96 (1H, m), 2.00 (2H, td, J=9.58, 5.48 Hz), 2.60-2.67 (1H, m), 2.62 (3H, s), 2.98-3.05 (1H, m), 3.12 (3H, d, J=4.65 Hz), 3.51 (1H, quin, J=6.30 Hz), 3.78-3.91 (1H, m), 4.42 (1H, t, J=6.16 Hz), 4.45 (1H, t, J=6.16 Hz), 4.50-4.66 (1H, m), 4.54 (2H, t, J=6.57 Hz), 4.78 (2H, td, J=15.81, 2.60 Hz), 6.32 (1H, d, J=8.76 Hz), 6.64 (1H, tt, J=54.30, 3.00 Hz), 7.09 (1H, d, J=3.01 Hz), 7.37 (1H, d, J=3.01 Hz), 7.85 (1H, d, J=8.49 Hz), 8.00 (1H, d, J=8.49 Hz), 10.96 (1H, q, J=4.65 Hz); ESIMS found for C₂₄H₂₈F₃N₉O m/z 516.3 (M+1).

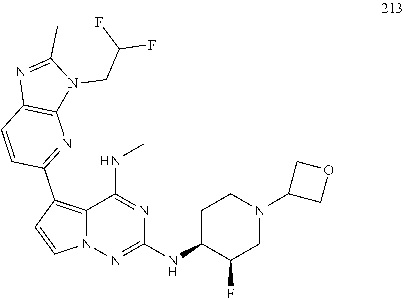 [SMILES: CNc1nc(N[C@H]2CCN(C3COC3)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4S)-3-fluoro-1-(oxetan-3-yl)piperidin-4-yl)-N⁴-methylpyrrolo[2,1-f][1,2,4]triazine-2,4-diamine 213

White solid (48 mg, 0.093 mmol, 44.1% yield). ¹H NMR (499 MHz, DMSO-d₆) δ ppm 1.67-1.76 (1H, m), 1.87 (1H, qd, J=12.09, 3.42 Hz), 1.96-2.05 (1H, m), 2.06-2.22 (1H, m), 2.62 (3H, s), 2.75 (1H, br d, J=10.40 Hz), 2.97 (1H, br t, J=10.27 Hz), 3.12 (3H, d, J=4.65 Hz), 3.48 (1H, quin, J=6.37 Hz), 3.71-3.89 (1H, m), 4.43 (2H, dt, J=29.35, 6.16 Hz), 4.54 (2H, td, J=6.50, 3.15 Hz), 4.72-4.82 (2H, m), 4.90 (1H, d, J=49.90 Hz), 5.95 (1H, d, J=8.21 Hz), 6.64 (1H, tt, J=54.30, 3.00 Hz), 7.10 (1H, d, J=3.01 Hz), 7.36 (1H, d, J=3.01 Hz), 7.85 (1H, d, J=8.76 Hz), 8.00 (1H, d, J=8.49 Hz), 10.97 (1H, q, J=4.47 Hz); ESIMS found for C₂₄H₂₈F₃N₉O m/z 516.3 (M+1).

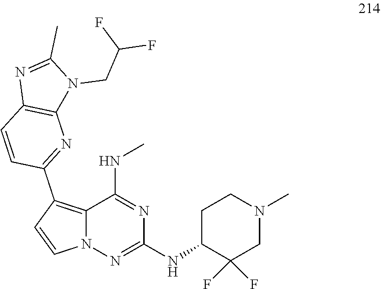 [SMILES: CNc1nc(N[C@@H]2CCN(C)CC2(F)F)nn2ccc(-c3ccc4nc(C)n(CC(F)F)c4n3)c12]

### (R)—N²-(3,3-Difluoro-1-methylpiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 214

Yellow solid (7 mg, 0.014 mmol, 32.4% yield). ¹H NMR (499 MHz, DMSO-d₆) δ ppm 1.72-1.89 (2H, m), 2.11-2.21 (1H, m), 2.26 (3H, br s), 2.32-2.46 (1H, m), 2.62 (3H, s), 2.74-2.84 (1H, m), 2.98-3.10 (1H, m), 3.14 (3H, d, J=4.65 Hz), 4.16-4.31 (1H, m), 4.73-4.84 (2H, m), 6.12 (1H, br d, J=9.03 Hz), 6.64 (1H, tt, J=54.30, 3.00 Hz), 7.11 (1H, d, J=3.01 Hz), 7.38 (1H, d, J=3.01 Hz), 7.85 (1H, d, J=8.76 Hz), 8.01 (1H, d, J=8.49 Hz), 10.99 (1H, q, J=4.47 Hz); ESIMS found for C₂₂H₂₅F₄N₉ m/z 492.3 (M+1).

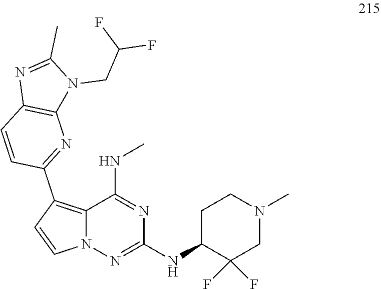 [SMILES: CNc1nc(N[C@H]2CCN(C)CC2(F)F)nn2ccc(-c3ccc4nc(C)n(CC(F)F)c4n3)c12]

### (S)—N²-(3,3-Difluoro-1-methylpiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 215

Yellow solid (8 mg, 0.016 mmol, 37.0% yield). ¹H NMR (499 MHz, DMSO-d₆) δ ppm 1.72-1.88 (2H, m), 2.10-2.22 (1H, m), 2.27 (3H, brs), 2.36 (1H, brs), 2.62 (3H, s), 2.75-2.85 (1H, m), 2.99-3.10 (1H, m), 3.14 (3H, d, J=4.65 Hz), 4.18-4.34 (1H, m), 4.78 (2H, td, J=15.74, 2.19 Hz), 6.13 (1H, br d, J=9.03 Hz), 6.64 (1H, tt, J=54.30, 3.00 Hz), 7.11 (1H, d, J=3.01 Hz), 7.38 (1H, d, J=3.01 Hz), 7.85 (1H, d, J=8.76 Hz), 8.01 (1H, d, J=8.49 Hz), 11.00 (1H, q, J=4.56 Hz); ESIMS found for C₂₂H₂₅F₄N₉ m/z 492.3 (M+1).

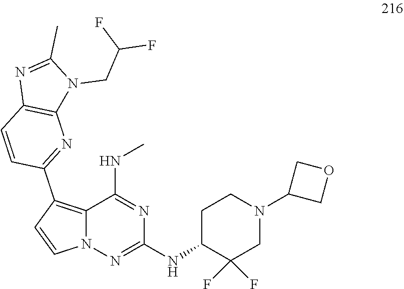 [SMILES: CNc1nc(N[C@@H]2CCN(C3COC3)CC2(F)F)nn2ccc(-c3ccc4nc(C)n(CC(F)F)c4n3)c12]

### (R)—N²-(3,3-Difluoro-1-(oxetan-3-yl)piperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 216

Yellow solid (12 mg, 0.023 mmol, 27.2% yield). ¹H NMR (499 MHz, DMSO-d₆) δ ppm 1.70-1.82 (1H, m), 1.88 (1H, br dd, J=9.03, 4.38 Hz), 2.14 (1H, br t, J=10.54 Hz), 2.32-2.44 (1H, m), 2.62 (3H, s), 2.74 (1H, brd, J=11.77 Hz), 2.95-3.06 (1H, m), 3.14 (3H, d, J=4.65 Hz), 3.60 (1H, quin, J=6.30 Hz), 4.22-4.36 (1H, m), 4.44 (2H, dt, J=14.24, 6.30 Hz), 4.55 (2H, td, J=6.64, 3.42 Hz), 4.73-4.85 (2H, m), 6.19 (1H, d, J=9.58 Hz), 6.64 (1H, tt, J=54.30, 3.00 Hz), 7.11 (1H, d, J=2.74 Hz), 7.37 (1H, d, J=2.74 Hz), 7.85 (1H, d, J=8.49 Hz), 8.01 (1H, d, J=8.49 Hz), 11.00 (1H, q, J=4.47 Hz); ESIMS found for C₂₄H₂₇F₄N₉O m/z 534.3 (M+1).

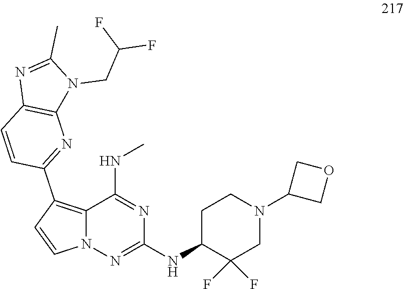 [SMILES: CNc1nc(N[C@H]2CCN(C3COC3)CC2(F)F)nn2ccc(-c3ccc4nc(C)n(CC(F)F)c4n3)c12]

### (S)—N²-(3,3-Difluoro-1-(oxetan-3-yl)piperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 217

Yellow solid (11 mg, 0.021 mmol, 29.7% yield). ¹H NMR (499 MHz, DMSO-d₆) δ ppm 1.70-1.82 (1H, m), 1.88 (1H, br dd, J=9.17, 4.52 Hz), 2.14 (1H, br t, J=10.68 Hz), 2.32-2.44 (1H, m), 2.62 (3H, s), 2.75 (1H, brd, J=11.50 Hz), 2.95-3.05 (1H, m), 3.14 (3H, d, J=4.93 Hz), 3.60 (1H, quin, J=6.30 Hz), 4.21-4.36 (1H, m), 4.44 (2H, dt, J=14.31, 6.26 Hz), 4.55 (2H, td, J=6.64, 3.42 Hz), 4.72-4.85 (2H, m), 6.19 (1H, d, J=9.58 Hz), 6.64 (1H, tt, J=54.30, 3.00 Hz), 7.11 (1H, d, J=3.01 Hz), 7.37 (1H, d, J=2.74 Hz), 7.85 (1H, d, J=8.76 Hz), 8.01 (1H, d, J=8.49 Hz), 11.00 (1H, q, J=4.75 Hz); ESIMS found for C₂₄H₂₇F₄N₉O m/z 534.3 (M+1).

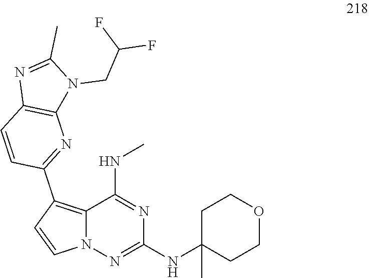 [SMILES: CNc1nc(NC2(C)CCOCC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-(4-methyltetrahydro-2H-pyran-4-yl)pyrrolo[2,1-f][1,2,4]triazine-2,4-diamine 218

White solid (4.0 mg, 0.009 mmol, 4.1% yield). ¹H NMR (499 MHz, DMSO-d₆) δ ppm 1.43 (3H, s), 1.54-1.65 (2H, m), 2.26 (2H, br d, J=13.69 Hz), 2.62 (3H, s), 3.12 (3H, d, J=4.65 Hz), 3.55-3.60 (2H, m), 3.61-3.66 (2H, m), 4.72-4.83 (2H, m), 5.61 (1H, s), 6.64 (1H, tt, J=54.30, 3.00 Hz), 7.09 (1H, d, J=3.01 Hz), 7.34 (1H, d, J=2.74 Hz), 7.84 (1H, d, J=8.49 Hz), 8.00 (1H, d, J=8.49 Hz), 10.84-10.93 (1H, m); ESIMS found for C₂₂H₂₆F₂N₈O m/z 457.2 (M+1).

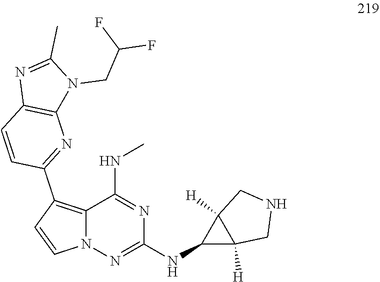 [SMILES: CNc1nc(N[C@@H]2[C@@H]3CNC[C@@H]32)nn2ccc(-c3ccc4nc(C)n(CC(F)F)c4n3)c12]

### N²-((1R,5S,6r)-3-Azabicyclo[3.1.0]hexan-6-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 219

Off-white solid (65 mg, 0.148 mmol, 61.4% yield). ¹H NMR (499 MHz, DMSO-d₆) δ ppm 1.64 (2H, br d, J=7.12 Hz), 2.62 (3H, s), 2.88-2.92 (1H, m), 2.92-2.97 (2H, m), 2.99-3.04 (2H, m), 3.11 (3H, d, J=4.65 Hz), 4.71-4.83 (2H, m), 6.04 (1H, d, J=4.11 Hz), 6.63 (1H, tt, J=54.30, 3.00 Hz), 7.10 (1H, d, J=2.74 Hz), 7.39 (1H, d, J=2.46 Hz), 7.85 (1H, d, J=8.49 Hz), 8.00 (1H, d, J=8.49 Hz), 10.93 (1H, br q, J=4.65 Hz); ESIMS found for C₂₁H₂₃F₂N₉ m/z 440.2 (M+1).

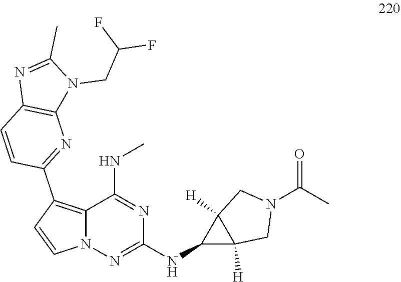 [SMILES: CNc1nc(N[C@@H]2[C@@H]3CN(C(C)=O)C[C@@H]32)nn2ccc(-c3ccc4nc(C)n(CC(F)F)c4n3)c12]

### 1-((1R,5S,6r)-6-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-3-azabicyclo[3.1.0]hexan-3-yl)ethan-1-one 220

Off-white solid (24 mg, 0.050 mmol, 73.0% yield). ¹H NMR (499 MHz, DMSO-d₆) δ ppm 1.83 (3H, s), 1.84-1.88 (1H, m), 1.89-1.95 (1H, m), 2.62 (3H, s), 2.69 (1H, td, J=6.91, 1.23 Hz), 3.12 (3H, d, J=4.65 Hz), 3.40-3.49 (2H, m), 3.54 (1H, d, J=10.40 Hz), 3.67 (1H, dd, J=10.40, 5.48 Hz), 4.72-4.83 (2H, m), 6.23 (1H, s), 6.63 (1H, tt, J=54.30, 3.00 Hz), 7.11 (1H, d, J=3.01 Hz), 7.40 (1H, d, J=2.74 Hz), 7.85 (1H, d, J=8.76 Hz), 8.01 (1H, d, J=8.49 Hz), 10.94 (1H, q, J=4.56 Hz); ESIMS found for C₂₃H₂₅F₂N₉O m/z 482.2 (M+1).

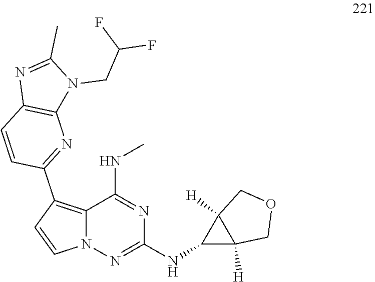 [SMILES: CNc1nc(N[C@H]2[C@@H]3COC[C@@H]32)nn2ccc(-c3ccc4nc(C)n(CC(F)F)c4n3)c12]

### N²-((1R,5S,6r)-3-Oxabicyclo[3.1.0]hexan-6-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 221

Off-white solid (12 mg, 0.027 mmol, 14.4% yield). ¹H NMR (499 MHz, DMSO-d₆) δ ppm 1.81 (2H, s), 2.40 (1H, q, J=2.46 Hz), 2.62 (3H, s), 3.10 (3H, d, J=4.65 Hz), 3.64 (2H, d, J=8.21 Hz), 3.86 (2H, d, J=8.21 Hz), 4.77 (2H, td, J=15.95, 2.33 Hz), 6.42 (1H, d, J=3.29 Hz), 6.63 (1H, tt, J=54.30, 3.00 Hz), 7.10 (1H, d, J=2.74 Hz), 7.39 (1H, d, J=3.01 Hz), 7.85 (1H, d, J=8.49 Hz), 8.00 (1H, d, J=8.49 Hz), 10.93 (1H, q, J=4.56 Hz); ESIMS found for C₂₁H₂₂F₂N₈O m/z 441.2 (M+1).

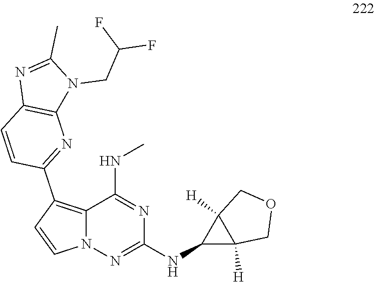 [SMILES: CNc1nc(N[C@@H]2[C@@H]3COC[C@@H]32)nn2ccc(-c3ccc4nc(C)n(CC(F)F)c4n3)c12]

### N²-((1R,5S,6s)-3-Oxabicyclo[3.1.0]hexan-6-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 222

Off-white solid (38 mg, 0.086 mmol, 32.6% yield). ¹H NMR (499 MHz, DMSO-d₆) δ ppm 1.96 (2H, br d, J=6.57 Hz), 2.62 (3H, s), 2.91 (1H, td, J=6.84, 4.11 Hz), 3.12 (3H, d, J=4.65 Hz), 3.81-3.88 (4H, m), 4.72-4.83 (2H, m), 5.64 (1H, d, J=4.11 Hz), 6.64 (1H, tt, J=54.18, 2.75 Hz), 7.10 (1H, d, J=3.01 Hz), 7.39 (1H, d, J=3.01 Hz), 7.85 (1H, d, J=8.76 Hz), 8.00 (1H, d, J=8.49 Hz), 10.94 (1H, q, J=4.65 Hz); ESIMS found for C₂₁H₂₂F₂N₈O m/z 441.1 (M+1).

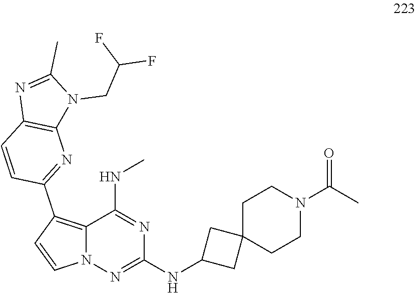 [SMILES: CNc1nc(NC2CC3(CCN(C(C)=O)CC3)C2)nn2ccc(-c3ccc4nc(C)n(CC(F)F)c4n3)c12]

### 1-(2-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-7-azaspiro[3.5]nonan-7-yl)ethan-1-one 223

Off-white solid (45 mg, 0.086 mmol, 90.0% yield). ¹H NMR (499 MHz, DMSO-d₆) δ ppm 1.40-1.45 (1H, m), 1.46-1.54 (2H, m), 1.55-1.61 (1H, m), 1.76 (2H, br t, J=8.76 Hz), 1.97 (3H, d, J=8.76 Hz), 2.23 (2H, br dd, J=9.58, 8.21 Hz), 2.62 (3H, s), 3.11 (3H, d, J=4.65 Hz), 3.26-3.31 (2H, m), 3.36-3.44 (2H, m), 4.13-4.25 (1H, m), 4.72-4.83 (2H, m), 6.40 (1H, t, J=7.26 Hz), 6.63 (1H, tt, J=54.30, 3.00 Hz), 7.08 (1H, d, J=3.01 Hz), 7.36 (1H, d, J=3.01 Hz), 7.84 (1H, d, J=8.49 Hz), 8.00 (1H, d, J=8.49 Hz), 10.91 (1H, q, J=4.47 Hz); ESIMS found for C₂₆H₃₁F₂N₉O m/z 524.3 (M+1).

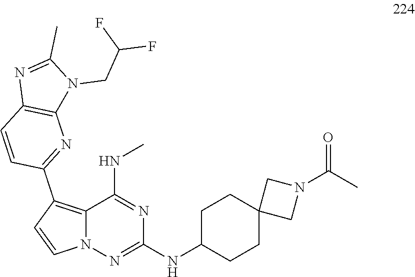 [SMILES: CNc1nc(NC2CCC3(CC2)CN(C(C)=O)C3)nn2ccc(-c3ccc4nc(C)n(CC(F)F)c4n3)c12]

### 1-(7-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-2-azaspiro[3.5]nonan-2-yl)ethan-1-one 224

Off-white solid (48 mg, 0.092 mmol, 84.9% yield). ¹H NMR (499 MHz, DMSO-d₆) δ ppm 0.94 (4H, d, J=6.57 Hz), 1.22-1.38 (2H, m), 1.46-1.59 (2H, m), 1.75 (3H, d, J=9.03 Hz), 1.82-1.92 (4H, m), 2.62 (3H, s), 3.11 (3H, d, J=4.11 Hz), 3.46 (1H, s), 3.51 (1H, s), 3.55 (1H, br d, J=8.49 Hz), 3.74 (1H, s), 3.79 (1H, s), 4.77 (2H, td, J=15.95, 2.33 Hz), 5.88 (1H, dd, J=19.16, 8.21 Hz), 6.63 (1H, tt, J=54.30, 3.00 Hz), 7.08 (1H, d, J=3.01 Hz), 7.36 (1H, t, J=2.74 Hz), 7.81-7.86 (1H, m), 8.00 (1H, d, J=8.49 Hz), 10.87-10.93 (1H, m); ESIMS found for C₂₆H₃₁F₂N₉O m/z 524.3 (M+1).

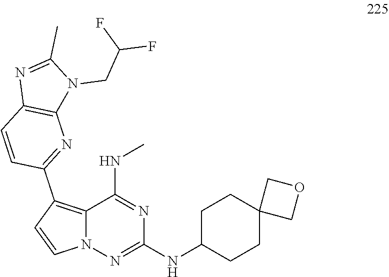 [SMILES: CNc1nc(NC2CCC3(CC2)COC3)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-(2-oxaspiro[3.5]nonan-7-yl)pyrrolo[2,1-f][1,2,4]triazine-2,4-diamine 225

White solid (31 mg, 0.064 mmol, 20.2% yield). ¹H NMR (499 MHz, DMSO-d₆) δ ppm 1.17-1.30 (2H, m), 1.51 (2H, td, J=12.94, 3.42 Hz), 1.85 (2H, br dd, J=12.87, 3.01 Hz), 2.05 (2H, br d, J=13.14 Hz), 2.62 (3H, s), 3.10 (3H, d, J=4.65 Hz), 3.47-3.59 (1H, m), 4.23 (2H, s), 4.31 (2H, s), 4.77 (2H, td, J=15.81, 2.05 Hz), 5.86 (1H, d, J=8.21 Hz), 6.63 (1H, tt, J=54.30, 3.00 Hz), 7.07 (1H, d, J=3.01 Hz), 7.35 (1H, d, J=3.01 Hz), 7.83 (1H, d, J=8.76 Hz), 7.99 (1H, d, J=8.49 Hz), 10.89 (1H, q, J=4.29 Hz); ESIMS found for C₂₄H₂₈F₂N₈O m/z 483.3 (M+1).

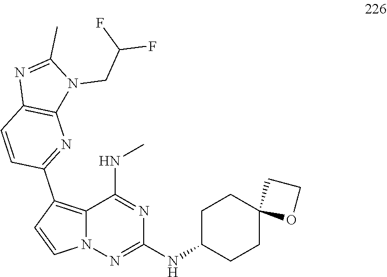 [SMILES: CNc1nc(N[C@H]2CC[C@@]3(CCO3)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-((4r,7r)-1-oxaspiro[3.5]nonan-7-yl)pyrrolo[2,1-f][1,2,4]triazine-2,4-diamine 226

Beige solid (15.6 mg, 0.032 mmol, 10.2% yield). ¹H NMR (400 MHz, DMSO-d₆) δ ppm 1.51 (3H, td, J=12.53, 3.30 Hz), 1.85-1.95 (2H, m), 2.04 (2H, br d, J=12.23 Hz), 2.31-2.37 (3H, m), 2.62 (3H, s), 3.11 (3H, d, J=4.52 Hz), 3.49-3.62 (2H, m), 4.37 (2H, t, J=7.70 Hz), 4.69-4.86 (2H, m), 5.90 (1H, br d, J=8.07 Hz), 6.63 (1H, tt, J=54.30, 3.00 Hz), 7.08 (1H, d, J=2.93 Hz), 7.37 (1H, d, J=2.81 Hz), 7.84 (1H, d, J=8.68 Hz), 8.00 (1H, d, J=8.44 Hz), 10.89 (1H, q, J=3.99 Hz); ESIMS found for C₂₄H₂₈F₂N₈O m/z 483.1 (M+1).

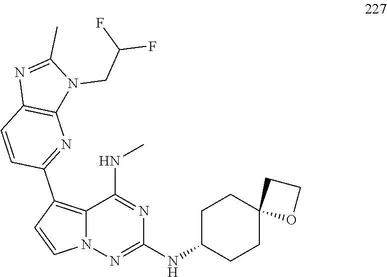 [SMILES: CNc1nc(N[C@H]2CC[C@]3(CCO3)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-((4s,7s)-1-oxaspiro[3.5]nonan-7-yl)pyrrolo[2,1-f][1,2,4]triazine-2,4-diamine 227

Off-white solid (8.77 mg, 0.018 mmol, 5.7% yield). ¹H NMR (400 MHz, DMSO-d₆) δ ppm 1.88-2.02 (2H, m), 2.03-2.18 (1H, m), 2.10 (3H, brt, J=6.72 Hz), 2.62 (3H, s), 3.11 (3H, d, J=4.65 Hz), 3.43-3.55 (2H, m), 3.70-3.84 (1H, m), 4.40 (1H, t, J=5.32 Hz), 4.77 (2H, td, J=15.93, 2.14 Hz), 5.36 (1H, br s), 5.91 (1H, d, J=8.31 Hz), 6.63 (1H, tt, J=54.30, 3.00 Hz), 7.08 (1H, d, J=3.06 Hz), 7.36 (1H, d, J=2.93 Hz), 7.84 (1H, d, J=8.68 Hz), 8.00 (1H, d, J=8.56 Hz), 10.90 (1H, q, J=4.28 Hz); ESIMS found for C₂₄H₂₈F₂N₈O m/z 483.1 (M+1).

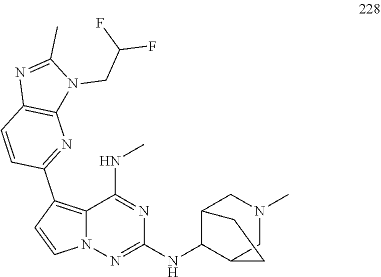 [SMILES: CNc1nc(NC2C3CCC2CN(C)C3)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-(3-methyl-3-azabicyclo[3.2.1]octan-8-yl)pyrrolo[2,1-f][1,2,4]triazine-2,4-diamine 228

White solid (25.0 mg, 0.052 mmol, 24.5% yield). ¹H NMR (499 MHz, DMSO-d₆) δ ppm 1.54-1.67 (4H, m), 1.75 (2H, br s), 1.94-2.02 (2H, m), 2.24 (3H, br s), 2.62 (3H, s), 3.11 (3H, d, J=4.65 Hz), 3.12-3.16 (2H, m), 3.88-4.01 (1H, m), 4.71-4.83 (2H, m), 5.88 (1H, br d, J=8.21 Hz), 6.63 (1H, tt, J=54.30, 3.00 Hz), 7.07 (1H, d, J=3.01 Hz), 7.33 (1H, d, J=2.74 Hz), 7.83 (1H, d, J=8.49 Hz), 7.99 (1H, d, J=8.76 Hz), 10.86-10.94 (1H, m); ESIMS found for C₂₄H₂₉F₂N₉ m/z 482.3 (M+1).

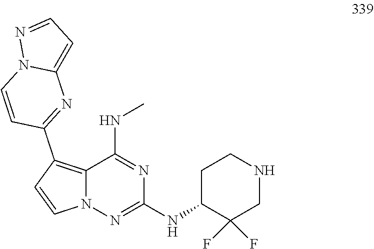 [SMILES: CNc1nc(N[C@@H]2CCNCC2(F)F)nn2ccc(-c3ccn4nccc4n3)c12]

### (R)—N²-(3,3-Difluoropiperidin-4-yl)-N⁴-methyl-5-(pyrazolo[1,5-a]pyrimidin-5-yl)pyrrolo[2,1-f][1,2,4]triazine-2,4-diamine 339

Yellow solid (38 mg, 0.095 mmol, 86.4% yield). ¹H NMR (499 MHz, DMSO-d₆) δ ppm 1.66 (1H, qd, J=11.82, 4.24 Hz), 1.78-1.89 (1H, m), 2.57 (1H, br t, J=11.50 Hz), 2.78 (1H, dd, J=29.30, 13.70 Hz), 2.91 (1H, br d, J=13.42 Hz), 3.06-3.12 (1H, m), 3.13 (3H, d, J=4.65 Hz), 4.24-4.41 (1H, m), 6.35 (1H, d, J=9.58 Hz), 6.68-6.74 (1H, m), 7.33 (1H, d, J=3.01 Hz), 7.43 (1H, d, J=3.01 Hz), 7.56 (1H, d, J=7.67 Hz), 8.17 (1H, d, J=2.19 Hz), 9.00-9.08 (1H, m), 11.52 (1H, q, J=4.56 Hz); ESIMS found for C₁₈H₁₉F₂N₉ m/z 400.2 (M+1).

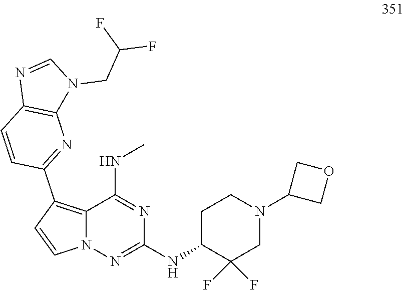 [SMILES: CNc1nc(N[C@@H]2CCN(C3COC3)CC2(F)F)nn2ccc(-c3ccc4ncn(CC(F)F)c4n3)c12]

### (R)—N²-(3,3-Difluoro-1-(oxetan-3-yl)piperidin-4-yl)-5-(3-(2,2-difluoroethyl)-3H-imidazo[4,5-b]pyridin-5-yl)-N⁴-methylpyrrolo[2,1-f][1,2,4]triazine-2,4-diamine 351

Yellowish white solid (6 mg, 0.012 mmol, 20.6% yield). ¹H NMR (499 MHz, DMSO-d₆) δ ppm 1.71-1.82 (1H, m), 1.83-1.91 (1H, m), 2.09-2.22 (1H, m), 2.37 (1H, dd, J=27.05, 11.85 Hz), 2.74 (1H, br d, J=11.77 Hz), 2.94-3.04 (1H, m), 3.10 (3H, d, J=4.65 Hz), 3.59 (1H, quin, J=6.30 Hz), 4.21-4.36 (1H, m), 4.44 (2H, dt, J=14.24, 6.16 Hz), 4.55 (2H, td, J=6.57, 3.29 Hz), 4.89 (2H, td, J=16.08, 2.60 Hz), 6.17 (1H, d, J=9.58 Hz), 6.49 (1H, tt, J=54.30, 3.00 Hz), 7.15 (1H, d, J=3.01 Hz), 7.36 (1H, d, J=2.74 Hz), 7.94 (1H, d, J=8.76 Hz), 8.18 (1H, d, J=8.76 Hz), 8.48 (1H, s), 12.24 (1H, q, J=4.29 Hz); ESIMS found for C₂₃H₂₅F₄N₉O m/z 520.2 (M+1).

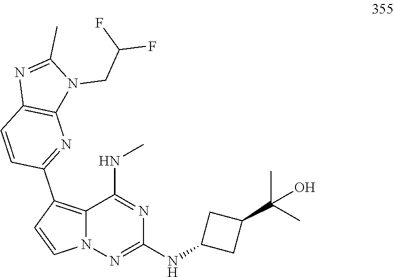 [SMILES: CNc1nc(N[C@H]2C[C@H](C(C)(C)O)C2)nn2ccc(-c3ccc4nc(C)n(CC(F)F)c4n3)c12]

### 2-(trans-3-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)cyclobutyl)propan-2-ol 355

Brown solid (33 mg, 0.070 mmol, 35.3% yield). ¹H NMR (499 MHz, DMSO-d₆) δ ppm 1.04 (6H, s), 1.88-1.98 (2H, m), 2.15-2.23 (1H, m), 2.24-2.33 (2H, m), 2.62 (3H, s), 3.11 (3H, d, J=4.65 Hz), 4.08 (1H, dq, J=14.03, 7.00 Hz), 4.14 (1H, s), 4.72-4.83 (2H, m), 6.33 (1H, d, J=7.12 Hz), 6.63 (1H, tt, J=54.30, 3.00 Hz), 7.07 (1H, d, J=3.01 Hz), 7.35 (1H, d, J=3.01 Hz), 7.83 (1H, d, J=8.49 Hz), 7.99 (1H, d, J=8.49 Hz), 10.88 (1H, q, J=4.56 Hz); ESIMS found for C₂₃H₂₈F₂N₈O m/z 471.3 (M+1).

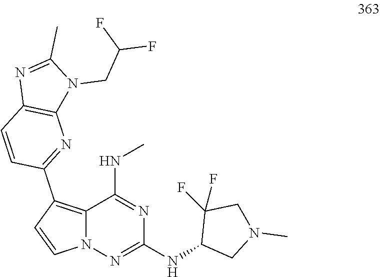 [SMILES: CNc1nc(N[C@H]2CN(C)CC2(F)F)nn2ccc(-c3ccc4nc(C)n(CC(F)F)c4n3)c12]

### (S)—N²-(4,4-Difluoro-1-methylpyrrolidin-3-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 363

Off-white solid (29 mg, 0.061 mmol, 38.2% yield). ¹H NMR (499 MHz, DMSO-d₆) δ ppm 2.27 (3H, s), 2.39 (1H, t, J=9.03 Hz), 2.55 (1H, dt, J=15.13, 3.80 Hz), 2.62 (3 H, s), 3.13 (3H, d, J=4.65 Hz), 3.19-3.25 (2H, m), 4.53-4.69 (1H, m), 4.73-4.85 (2H, m), 6.39 (1H, d, J=9.03 Hz), 6.64 (1H, tt, J=54.30, 3.00 Hz), 7.13 (1H, d, J=3.01 Hz), 7.38 (1H, d, J=3.01 Hz), 7.86 (1H, d, J=8.49 Hz), 8.01 (1H, d, J=8.49 Hz), 11.02 (1H, q, J=4.38 Hz); ESIMS found for C₂₁H₂₃F₄N₉ m/z 478.2 (M+1).

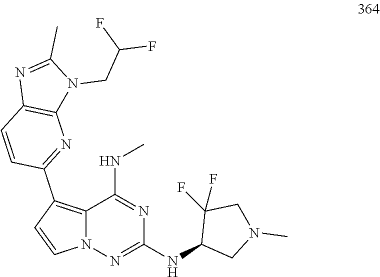 [SMILES: CNc1nc(N[C@@H]2CN(C)CC2(F)F)nn2ccc(-c3ccc4nc(C)n(CC(F)F)c4n3)c12]

### (R)—N²-(4,4-Difluoro-1-methylpyrrolidin-3-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 364

Off-white solid (19 mg, 0.040 mmol, 37.6% yield). ¹H NMR (499 MHz, DMSO-d₆) δ ppm 2.27 (3H, s), 2.39 (1H, t, J=9.03 Hz), 2.52-2.61 (1H, m), 2.62 (3H, s), 3.13 (3H, d, J=4.65 Hz), 3.18-3.26 (2H, m), 4.55-4.68 (1H, m), 4.78 (2H, td, J=16.02, 2.74 Hz), 6.39 (1H, d, J=9.31 Hz), 6.64 (1H, tt, J=54.30, 3.00 Hz), 7.13 (1H, d, J=3.01 Hz), 7.38 (1H, d, J=2.74 Hz), 7.86 (1H, d, J=8.76 Hz), 8.01 (1H, d, J=8.49 Hz), 11.02 (1H, q, J=4.47 Hz); ESIMS found for C₂₁H₂₃F₄N₉ m/z 478.2 (M+1).

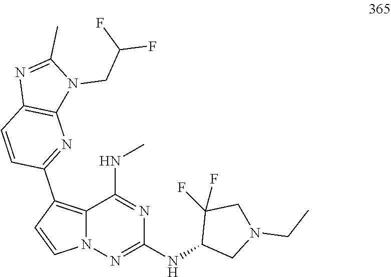 [SMILES: CCN1C[C@H](Nc2nc(NC)c3c(-c4ccc5nc(C)n(CC(F)F)c5n4)ccn3n2)C(F)(F)C1]

### (S)-5-(3-(2,2-Difluoroethyl)-2-methyl-3H-imidazo[4,5-b]pyridin-5-yl)-N²-(1-ethyl-4,4-difluoropyrrolidin-3-yl)-N⁴-methylpyrrolo[2,1-f][1,2,4]triazine-2,4-diamine 365

Pale-yellow fluffy solid (12 mg, 0.024 mmol, 15.4% yield). ¹H NMR (499 MHz, DMSO-d₆) δ ppm 1.03 (3H, t, J=7.26 Hz), 2.35-2.41 (1H, m), 2.41-2.48 (2H, m), 2.56 (1H, ddd, J=18.69, 15.40, 11.09 Hz), 2.62 (3H, s), 3.13 (3H, d, J=4.65 Hz), 3.27 (2H, brt, J=8.62 Hz), 4.60 (1H, dt, J=15.19, 7.73 Hz), 4.78 (2H, td, J=16.08, 2.60 Hz), 6.38 (1H, d, J=9.03 Hz), 6.64 (1H, tt, J=54.30, 3.00 Hz), 7.13 (1H, d, J=3.01 Hz), 7.38 (1H, d, J=3.01 Hz), 7.86 (1H, d, J=8.49 Hz), 8.01 (1H, d, J=8.49 Hz), 11.02 (1H, q, J=4.65 Hz); ESIMS found for C₂₂H₂₅F₄N₉ m/z 492.2 (M+1).

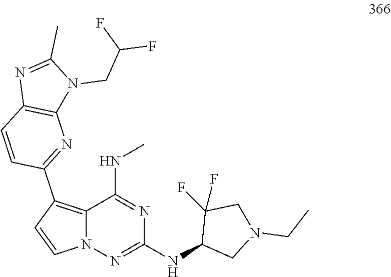 [SMILES: CCN1C[C@@H](Nc2nc(NC)c3c(-c4ccc5nc(C)n(CC(F)F)c5n4)ccn3n2)C(F)(F)C1]

### (R)-5-(3-(2,2-Difluoroethyl)-2-methyl-3H-imidazo[4,5-b]pyridin-5-yl)-N²-(1-ethyl-4,4-difluoropyrrolidin-3-yl)-N⁴-methylpyrrolo[2,1-f][1,2,4]triazine-2,4-diamine 366

Pale-yellow solid (11 mg, 0.022 mmol, 33.8% yield). ¹H NMR (499 MHz, DMSO-d₆) δ ppm 1.03 (3H, t, J=7.12 Hz), 2.35-2.41 (1H, m), 2.41-2.48 (2H, m), 2.51-2.61 (1H, m), 2.62 (3H, s), 3.13 (3H, d, J=4.65 Hz), 3.27 (2H, br t, J=8.62 Hz), 4.53-4.68 (1H, m), 4.78 (2H, td, J=16.02, 2.46 Hz), 6.38 (1H, d, J=9.03 Hz), 6.64 (1H, tt, J=54.30, 3.00 Hz), 7.13 (1H, d, J=3.01 Hz), 7.38 (1H, d, J=3.01 Hz), 7.86 (1H, d, J=8.76 Hz), 8.01 (1H, d, J=8.49 Hz), 11.02 (1H, q, J=4.75 Hz); ESIMS found for C₂₂H₂₅F₄N₉ m/z 492.2 (M+1).

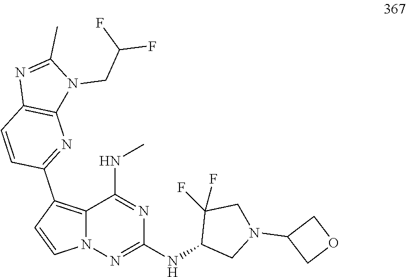 [SMILES: CNc1nc(N[C@H]2CN(C3COC3)CC2(F)F)nn2ccc(-c3ccc4nc(C)n(CC(F)F)c4n3)c12]

### (S)—N²-(4,4-difluoro-1-(oxetan-3-yl)pyrrolidin-3-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 367

Yellow solid (5 mg, 0.010 mmol, 9.1% yield). ¹H NMR (499 MHz, DMSO-d₆) δ ppm 2.44-2.49 (1H, m), 2.63 (3H, s), 2.65-2.75 (1H, m), 3.14 (3H, d, J=4.65 Hz), 3.27 (2H, brt, J=8.76 Hz), 3.68 (1H, quin, J=6.09 Hz), 4.46-4.52 (2H, m), 4.58 (2H, q, J=6.84 Hz), 4.61-4.71 (1H, m), 4.78 (2H, td, J=15.95, 2.60 Hz), 6.43 (1H, d, J=9.31 Hz), 6.64 (1H, tt, J=54.30, 3.00 Hz), 7.13 (1H, d, J=3.01 Hz), 7.39 (1H, d, J=3.01 Hz), 7.86 (1H, d, J=8.76 Hz), 8.01 (1H, d, J=8.49 Hz), 11.03 (1H, q, J=4.56 Hz); ESIMS found for C₂₃H₂₅F₄N₉O m/z 520.2 (M+1).

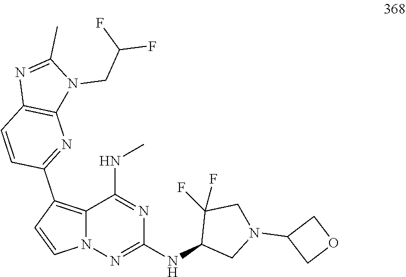 [SMILES: CNc1nc(N[C@@H]2CN(C3COC3)CC2(F)F)nn2ccc(-c3ccc4nc(C)n(CC(F)F)c4n3)c12]

### (R)—N²-(4,4-Difluoro-1-(oxetan-3-yl)pyrrolidin-3-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 368

Pale-orange solid (6 mg, 0.012 mmol, 7.3% yield). ¹H NMR (499 MHz, DMSO-d₆) δ ppm 2.45-2.49 (1H, m), 2.63 (3H, s), 2.65-2.75 (1H, m), 3.14 (3H, d, J=4.65 Hz), 3.22-3.30 (2H, m), 3.68 (1H, quin, J=6.02 Hz), 4.46-4.53 (2H, m), 4.58 (2H, q, J=6.75 Hz), 4.61-4.71 (1H, m), 4.79 (2H, td, J=15.88, 2.74 Hz), 6.43 (1H, d, J=9.03 Hz), 6.64 (1H, tt, J=54.30, 3.00 Hz), 7.13 (1H, d, J=3.01 Hz), 7.39 (1H, d, J=3.01 Hz), 7.86 (1H, d, J=8.76 Hz), 8.01 (1H, d, J=8.49 Hz), 11.03 (1H, q, J=4.65 Hz); ESIMS found for C₂₃H₂₅F₄N₉O m/z 520.2 (M+1).

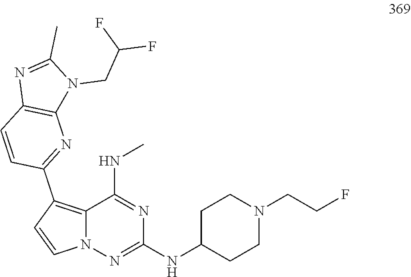 [SMILES: CNc1nc(NC2CCN(CCF)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-(1-(2-fluoroethyl)piperidin-4-yl)-N⁴-methylpyrrolo[2,1-f][1,2,4]triazine-2,4-diamine 369

Pale-yellow fluffy solid (29 mg, 0.060 mmol, 79.6% yield). ¹H NMR (499 MHz, DMSO-d₆) δ ppm 1.50 (2H, qd, J=11.73, 3.42 Hz), 1.83-1.93 (2H, m), 2.04-2.15 (2H, m), 2.60 (2H, dt, J=28.25, 4.93 Hz), 2.62 (3H, s), 2.87 (2H, br d, J=11.77 Hz), 3.11 (3H, d, J=4.65 Hz), 3.50-3.62 (1H, m), 4.52 (2H, dt, J=48.00, 4.95 Hz), 4.77 (2H, td, J=16.02, 2.74 Hz), 5.96 (1H, d, J=8.21 Hz), 6.63 (1H, tt, J=54.30, 3.00 Hz), 7.08 (1H, d, J=3.01 Hz), 7.36 (1H, d, J=2.74 Hz), 7.84 (1H, d, J=8.76 Hz), 8.00 (1H, d, J=8.49 Hz), 10.90 (1H, q, J=4.56 Hz); ESIMS found for C₂₃H₂₈F₃N₉ m/z 488.3 (M+1).

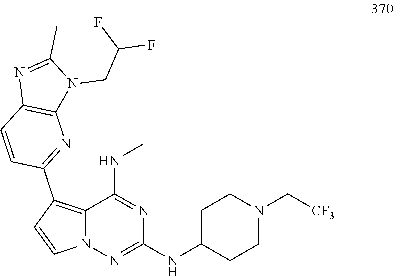 [SMILES: CNc1nc(NC2CCN(CC(F)(F)F)CC2)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-(1-(2,2,2-trifluoroethyl)piperidin-4-yl)pyrrolo[2,1-f][1,2,4]triazine-2,4-diamine 370

Pale-yellow fluffy solid (2 mg, 0.004 mmol, 5.1% yield). ¹H NMR (499 MHz, DMSO-d₆) δ ppm 1.44-1.60 (2H, m), 1.83-1.92 (2H, m), 2.42 (2H, br t, J=10.54 Hz), 2.62 (3H, s), 2.92 (2H, br d, J=12.05 Hz), 3.11 (3H, d, J=4.65 Hz), 3.15 (2H, q, J=10.15 Hz), 3.53-3.62 (1H, m), 4.69-4.85 (2H, m), 5.99 (1H, d, J=7.94 Hz), 6.63 (1H, tt, J=54.30, 3.00 Hz), 7.08 (1H, d, J=3.01 Hz), 7.36 (1H, d, J=3.01 Hz), 7.84 (1H, d, J=8.49 Hz), 8.00 (1H, d, J=8.49 Hz), 10.91 (1H, q, J=4.80 Hz); ESIMS found for C₂₃H₂₆F₅N₉ m/z 524.3 (M+1).

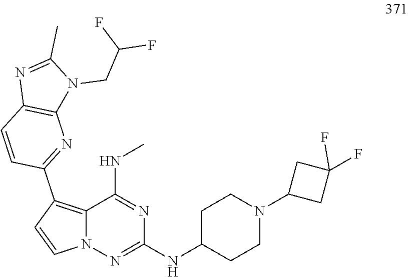 [SMILES: CNc1nc(NC2CCN(C3CC(F)(F)C3)CC2)nn2ccc(-c3ccc4nc(C)n(CC(F)F)c4n3)c12]

### N²-(1-(3,3-Difluorocyclobutyl)piperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 371

Pale yellow solid (5 mg, 0.009 mmol, 5.9% yield). ¹H NMR (499 MHz, DMSO-d₆) δ ppm 1.43-1.55 (2H, m), 1.84-1.94 (4H, m), 2.31-2.44 (2H, m), 2.62 (3H, s), 2.63-2.72 (3H, m), 2.78 (2H, br d, J=10.95 Hz), 3.11 (3H, d, J=4.65 Hz), 3.51-3.65 (1H, m), 4.71-4.83 (2H, m), 6.00 (1H, d, J=8.21 Hz), 6.63 (1H, tt, J=54.30, 3.00 Hz), 7.08 (1H, d, J=2.74 Hz), 7.36 (1H, d, J=2.74 Hz), 7.84 (1H, d, J=8.76 Hz), 8.00 (1H, d, J=8.49 Hz), 10.91 (1H, q, J=4.38 Hz); ESIMS found for C₅H₂₉F₄N₉ m/z 532.3 (M+1).

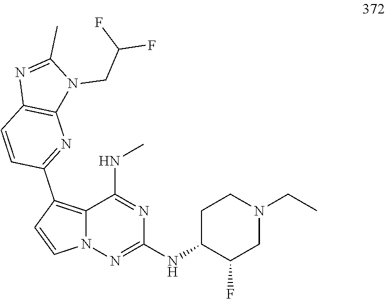 [SMILES: CCN1CC[C@@H](Nc2nc(NC)c3c(-c4ccc5nc(C)n(CC(F)F)c5n4)ccn3n2)[C@@H](F)C1]

### 5-(3-(2,2-Difluoroethyl)-2-methyl-3H-imidazo[4,5-b]pyridin-5-yl)-N²-((3S,4R)-1-ethyl-3-fluoropiperidin-4-yl)-N⁴-methylpyrrolo[2,1-f][1,2,4]triazine-2,4-diamine 372

Pale-yellow fluffy solid (4 mg, 0.008 mmol, 12.6% yield). ¹H NMR (499 MHz, DMSO-d₆) δ ppm 1.00 (3H, t, J=7.12 Hz), 1.64-1.74 (1H, m), 1.85 (1H, qd, J=12.27, 3.97 Hz), 1.98-2.08 (1H, m), 2.17 (1H, dd, J=38.15, 12.32 Hz), 2.36 (2H, q, J=7.21 Hz), 2.62 (3H, s), 2.88 (1H, brd, J=11.50 Hz), 3.09-3.16 (1H, m), 3.12 (3H, d, J=4.65 Hz), 3.67-3.87 (1H, m), 4.78 (2H, td, J=15.81, 2.33 Hz), 4.89 (1H, d, J=50.50 Hz), 5.87 (1H, d, J=8.21 Hz), 6.64 (1H, tt, J=54.30, 3.00 Hz), 7.10 (1H, d, J=3.01 Hz), 7.37 (1H, d, J=3.01 Hz), 7.85 (1H, d, J=8.76 Hz), 8.01 (1H, d, J=8.49 Hz), 10.97 (1H, q, J=4.38 Hz); ESIMS found for C₂₃H₂₈F₃N₉ m/z 488.3 (M+1).

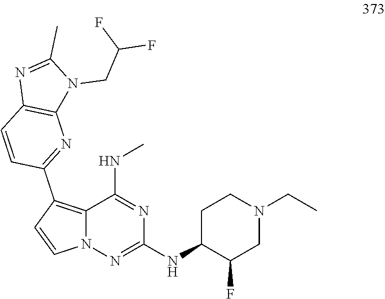 [SMILES: CCN1CC[C@H](Nc2nc(NC)c3c(-c4ccc5nc(C)n(CC(F)F)c5n4)ccn3n2)[C@H](F)C1]

### 5-(3-(2,2-Difluoroethyl)-2-methyl-3H-imidazo[4,5-b]pyridin-5-yl)-N²-((3R,4S)-1-ethyl-3-fluoropiperidin-4-yl)-N⁴-methylpyrrolo[2,1-f][1,2,4]triazine-2,4-diamine 373

Pale-yellow fluffy solid (6 mg, 0.012 mmol, 18.8% yield). ¹H NMR (499 MHz, DMSO-d₆) δ ppm 1.00 (3H, t, J=7.12 Hz), 1.64-1.75 (1H, m), 1.85 (1H, qd, J=12.18, 3.42 Hz), 1.99-2.08 (1H, m), 2.17 (1H, dd, J=37.85, 12.59 Hz), 2.36 (2H, q, J=7.12 Hz), 2.62 (3H, s), 2.88 (1H, brd, J=10.95 Hz), 3.08-3.16 (1H, m), 3.12 (3H, d, J=4.65 Hz), 3.68-3.85 (1H, m), 4.78 (2H, td, J=15.95, 2.33 Hz), 4.89 (1H, d, J=50.50 Hz), 5.87 (1H, d, J=8.21 Hz), 6.64 (1H, tt, J=54.30, 3.00 Hz), 7.10 (1H, d, J=2.74 Hz), 7.37 (1H, d, J=2.74 Hz), 7.85 (1H, d, J=8.49 Hz), 8.01 (1H, d, J=8.49 Hz), 10.97 (1H, J=4.47 Hz); ESIMS found for C₂₃H₂₈F₃N₉ m/z 488.3 (M+1).

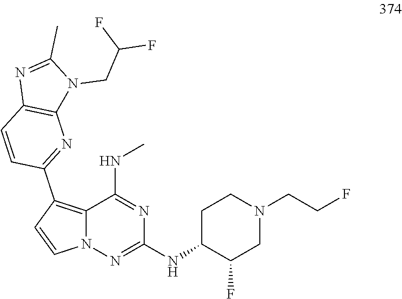 [SMILES: CNc1nc(N[C@@H]2CCN(CCF)C[C@@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3S,4R)-3-fluoro-1-(2-fluoroethyl)piperidin-4-yl)-N⁴-methylpyrrolo[2,1-f][1,2,4]triazine-2,4-diamine 374

Pale-yellow fluffy solid (29 mg, 0.057 mmol, 87.9% yield). ¹H NMR (499 MHz, DMSO-d₆) δ ppm 1.63-1.74 (1H, m), 1.82-1.93 (1H, m), 2.23 (1H, br t, J=11.09 Hz), 2.37 (1H, dd, J=37.85, 12.60 Hz), 2.62 (3H, s), 2.63-2.73 (2H, m), 2.92 (1H, br d, J=10.95 Hz), 3.12 (3H, d, J=4.65 Hz), 3.17 (1H, brt, J=10.54 Hz), 3.70-3.88 (1H, m), 4.54 (2H, dt, J=47.70, 4.93 Hz), 4.78 (2H, td, J=16.08, 2.60 Hz), 4.89 (1H, d, J=49.90 Hz), 5.91 (1H, br d, J=7.94 Hz), 6.64 (1H, tt, J=54.30, 3.00 Hz), 7.11 (1H, d, J=3.01 Hz), 7.37 (1H, d, J=2.74 Hz), 7.85 (1H, d, J=8.76 Hz), 8.01 (1H, d, J=8.49 Hz), 10.97 (1H, q, J=4.38 Hz); ESIMS found for C₂₃H₂₇F₄N₉ m/z 506.25 (M+1).

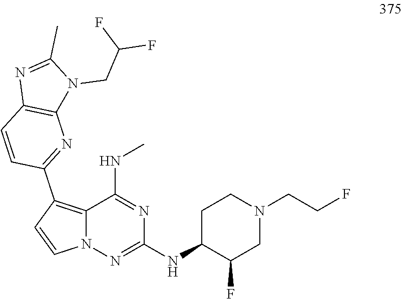 [SMILES: CNc1nc(N[C@H]2CCN(CCF)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4S)-3-fluoro-1-(2-fluoroethyl)piperidin-4-yl)-N⁴-methylpyrrolo[2,1-f][1,2,4]triazine-2,4-diamine 375

White solid (28 mg, 0.055 mmol, 84.8% yield). ¹H NMR (499 MHz, DMSO-d₆) δ ppm 1.63-1.73 (1H, m), 1.87 (1H, qd, J=12.27, 3.42 Hz), 2.23 (1H, br t, J=11.09 Hz), 2.37 (1H, dd, J=37.85, 12.90 Hz), 2.62 (3H, s), 2.67 (2H, dt, J=28.50, 4.95 Hz), 2.92 (1H, br d, J=11.50 Hz), 3.12 (3H, d, J=4.65 Hz), 3.13-3.20 (1H, m), 3.69-3.87 (1H, m), 4.54 (2H, dt, J=48.00, 4.95 Hz), 4.78 (2H, td, J=15.74, 2.46 Hz), 4.89 (1H, d, J=50.15 Hz), 5.90 (1H, d, J=8.21 Hz), 6.64 (1H, tt, J=54.30, 3.00 Hz), 7.11 (1H, d, J=3.01 Hz), 7.37 (1H, d, J=3.01 Hz), 7.85 (1H, d, J=8.49 Hz), 8.01 (1H, d, J=8.49 Hz), 10.97 (1H, q, J=4.29 Hz); ESIMS found for C₂₃H₂₇F₄N₉ m/z 506.3 (M+1).

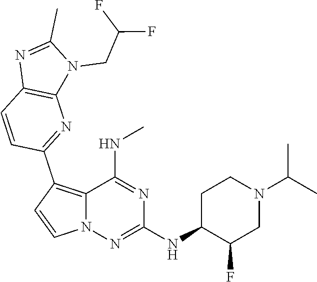 [SMILES: CNc1nc(N[C@H]2CCN(C(C)C)C[C@H]2F)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N²-((3R,4S)-3-fluoro-1-isopropylpiperidin-4-yl)-N⁴-methylpyrrolo[2,1-f][1,2,4]triazine-2,4-diamine 376

Pale-yellow fluffy solid (25 mg, 0.050 mmol, 76.3% yield). ¹H NMR (499 MHz, DMSO-d₆) δ ppm 0.96 (3H, d, J=5.20 Hz), 0.98 (3H, d, J=5.20 Hz), 1.66-1.73 (1H, m), 1.81 (1H, qd, J=11.96, 3.83 Hz), 2.25 (1H, br t, J=10.95 Hz), 2.39 (1H, dd, J=37.05, 12.35 Hz), 2.62 (3H, s), 2.73 (1H, dt, J=13.07, 6.47 Hz), 2.77-2.82 (1H, m), 3.01-3.08 (1H, m), 3.12 (3H, d, J=4.65 Hz), 3.67-3.83 (1H, m), 4.78 (2H, td, J=15.95, 2.60 Hz), 4.88 (1H, d, J=50.15 Hz), 5.85 (1H, d, J=8.21 Hz), 6.64 (1H, tt, J=54.30, 3.00 Hz), 7.10 (1H, d, J=3.01 Hz), 7.36 (1H, d, J=3.01 Hz), 7.85 (1H, d, J=8.76 Hz), 8.00 (1H, d, J=8.76 Hz), 10.97 (1H, q, J=4.38 Hz); ESIMS found for C₂₄H₃₀F₃N₉ m/z 502.3 (M+1).

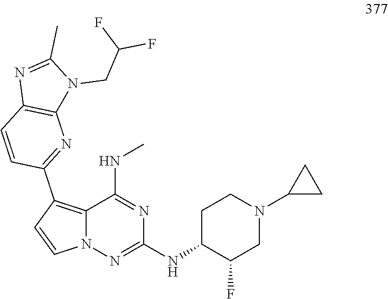 [SMILES: CNc1nc(N[C@@H]2CCN(C3CC3)C[C@@H]2F)nn2ccc(-c3ccc4nc(C)n(CC(F)F)c4n3)c12]

### N²-((3S,4R)-1-Cyclopropyl-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 377

Pale-yellow fluffy solid (14 mg, 0.028 mmol, 42.9% yield). ¹H NMR (499 MHz, DMSO-d₆) δ ppm 0.20-0.29 (1H, m), 0.30-0.37 (1H, m), 0.39-0.49 (2H, m), 1.61-1.72 (2H, m), 1.79 (1H, qd, J=12.32, 3.83 Hz), 2.37 (1H, dd, J=45.80, 13.15 Hz), 2.33-2.38 (1H, m), 2.62 (3H, s), 2.95 (1H, br d, J=11.77 Hz), 3.12 (3H, d, J=4.65 Hz), 3.14-3.24 (1H, m), 3.70-3.88 (1H, m), 4.78 (2H, td, J=16.08, 2.60 Hz), 4.87 (1H, d, J=50.20 Hz), 5.87 (1H, br d, J=7.94 Hz), 6.64 (1H, tt, J=54.30, 3.00 Hz), 7.11 (1H, d, J=3.01 Hz), 7.37 (1H, d, J=2.74 Hz), 7.85 (1H, d, J=8.76 Hz), 8.01 (1H, d, J=8.49 Hz), 10.97 (1H, q, J=4.56 Hz); ESIMS found for C₂₄H₂₈F₃N₉ m/z 500.3 (M+1).

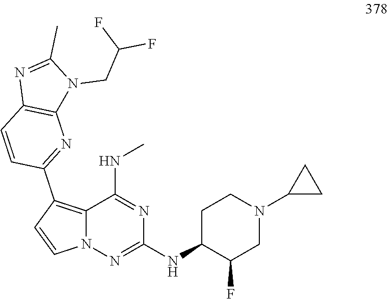 [SMILES: CNc1nc(N[C@H]2CCN(C3CC3)C[C@H]2F)nn2ccc(-c3ccc4nc(C)n(CC(F)F)c4n3)c12]

### N²-((3R,4S)-1-Cyclopropyl-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 378

Yellow solid (12 mg, 0.024 mmol, 36.8% yield). ¹HNMR (499 MHz, DMSO-d₆) δ ppm 0.21-0.28 (1H, m), 0.30-0.37 (1H, m), 0.43 (2H, br d, J=6.30 Hz), 1.61-1.72 (2H, m), 1.78 (1H, qd, J=12.18, 3.70 Hz), 2.29-2.44 (2H, m), 2.62 (3H, s), 2.95 (1H, br d, J=11.50 Hz), 3.12 (3H, d, J=4.65 Hz), 3.17 (1H, br t, J=10.95 Hz), 3.70-3.88 (1H, m), 4.73-4.81 (2H, m), 4.87 (1H, d, J=50.45 Hz), 5.86 (1H, d, J=8.21 Hz), 6.64 (1H, tt, J=54.30, 3.00 Hz), 7.10 (1H, d, J=3.01 Hz), 7.37 (1H, d, J=2.74 Hz), 7.85 (1H, d, J=8.49 Hz), 8.01 (1H, d, J=8.49 Hz), 10.97 (1H, q, J=4.65 Hz); ESIMS found for C₂₄H₂₈F₃N₉ m/z 500.3 (M+1).

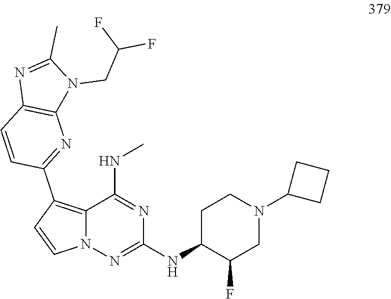 [SMILES: CNc1nc(N[C@H]2CCN(C3CCC3)C[C@H]2F)nn2ccc(-c3ccc4nc(C)n(CC(F)F)c4n3)c12]

### N2-((3R,4S)-1-Cyclobutyl-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 379

Pale-yellow fluffy solid (24 mg, 0.047 mmol, 71.6% yield). ¹H NMR (499 MHz, DMSO-d₆) δ ppm 1.58-1.65 (2H, m), 1.66-1.89 (5H, m), 1.92-2.07 (3H, m), 2.62 (3H, s), 2.74 (1H, quin, J=7.94 Hz), 2.80 (1H, br d, J=9.58 Hz), 2.99-3.07 (1H, m), 3.12 (3H, d, J=4.65 Hz), 3.69-3.84 (1H, m), 4.77 (2H, td, J=15.88, 2.46 Hz), 4.88 (1H, d, J=50.15 Hz), 5.88 (1H, d, J=8.21 Hz), 6.64 (1H, tt, J=54.30, 3.00 Hz), 7.10 (1H, d, J=3.01 Hz), 7.36 (1H, d, J=2.74 Hz), 7.85 (1H, d, J=8.76 Hz), 8.00 (1H, d, J=8.49 Hz), 10.97 (1H, q, J=4.56 Hz); ESIMS found for C₂₅H₃₀F₃N₉ m/z 514.3 (M+1).

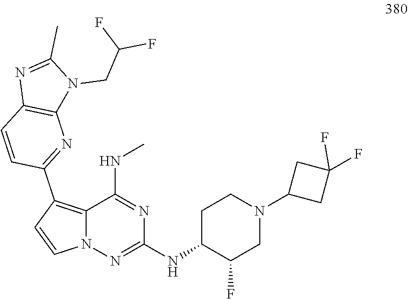 [SMILES: CNc1nc(N[C@@H]2CCN(C3CC(F)(F)C3)C[C@@H]2F)nn2ccc(-c3ccc4nc(C)n(CC(F)F)c4n3)c12]

### N²-((3S,4R)-1-(3,3-Difluorocyclobutyl)-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 380

Off-white fluffy solid (6 mg, 0.011 mmol, 7.7% yield). ¹H NMR (499 MHz, DMSO-d₆) δ ppm 1.66-1.75 (1H, m), 1.85 (1H, qd, J=12.18, 3.70 Hz), 1.97-2.08 (1H, m), 2.16 (1H, dd, J=37.55, 12.32 Hz), 2.28-2.47 (2H, m), 2.62 (3H, s), 2.64-2.77 (3H, m), 2.83 (1H, br d, J=10.13 Hz), 3.02-3.09 (1H, m), 3.12 (3H, d, J=4.65 Hz), 3.73-3.89 (1H, m), 4.78 (2H, td, J=15.74, 2.19 Hz), 4.91 (1H, d, J=49.90 Hz), 5.95 (1H, d, J=8.21 Hz), 6.64 (1H, tt, J=54.30, 3.00 Hz), 7.11 (1H, d, J=3.01 Hz), 7.37 (1H, d, J=3.01 Hz), 7.85 (1H, d, J=8.76 Hz), 8.01 (1H, d, J=8.49 Hz), 10.98 (1H, q, J=4.56 Hz); ESIMS found for C₂₅H₂₈F₅N₉ m/z 550.3 (M+1).

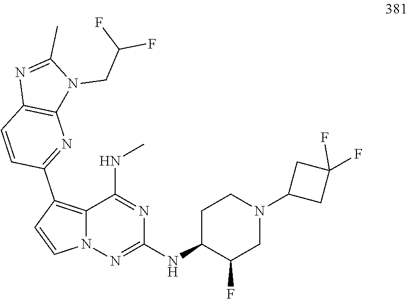 [SMILES: CNc1nc(N[C@H]2CCN(C3CC(F)(F)C3)C[C@H]2F)nn2ccc(-c3ccc4nc(C)n(CC(F)F)c4n3)c12]

### N²-((3R,4S)-1-(3,3-Difluorocyclobutyl)-3-fluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 381

Pale yellow solid (9 mg, 0.016 mmol, 25.1% yield). ¹H NMR (499 MHz, DMSO-d₆) δ ppm 1.67-1.74 (1H, m), 1.85 (1H, qd, J=12.18, 3.97 Hz), 1.98-2.06 (1H, m), 2.16 (1H, dd, J=37.30, 13.15 Hz), 2.28-2.46 (2H, m), 2.62 (3H, s), 2.66-2.77 (3H, m), 2.83 (1H, br d, J=10.13 Hz), 3.06 (1H, br t, J=10.95 Hz), 3.12 (3H, d, J=4.65 Hz), 3.73-3.89 (1H, m), 4.72-4.82 (2H, m), 4.91 (1H, d, J=49.05 Hz), 5.94 (1H, d, J=8.21 Hz), 6.64 (1H, tt, J=54.30, 3.00 Hz), 7.11 (1H, d, J=2.74 Hz), 7.37 (1H, d, J=3.01 Hz), 7.85 (1H, d, J=8.76 Hz), 8.01 (1H, d, J=8.49 Hz), 10.98 (1H, q, J=4.53 Hz); ESIMS found for C₂₅H₂₈F₅N₉ m/z 550.3 (M+1).

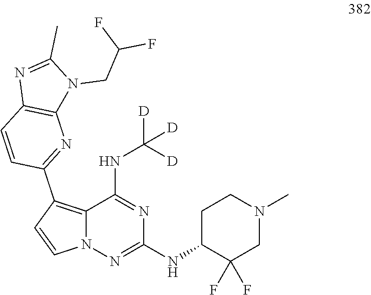 [SMILES: [2H]C([2H])([2H])Nc1nc(N[C@@H]2CCN(C)CC2(F)F)nn2ccc(-c3ccc4nc(C)n(CC(F)F)c4n3)c12]

### (R)—N²-(3,3-Difluoro-1-methylpiperidin-4-yl)-5-(3-(2,2-difluoroethyl)-2-methyl-3H-imidazo[4,5-b]pyridin-5-yl)-N⁴-(methyl-d₃)pyrrolo[2,1-f][1,2,4]triazine-2,4-diamine 382

Off-white solid (9 mg, 0.018 mmol, 43.7% yield). ¹H NMR (499 MHz, DMSO-d₆) δ ppm 1.72-1.81 (1H, m), 1.81-1.88 (1H, m), 2.10-2.21 (1H, m), 2.25 (3H, s), 2.36 (1H, ddd, J=27.20, 11.91, 1.12 Hz), 2.62 (3H, s), 2.77 (1H, br d, J=11.77 Hz), 2.98-3.10 (1H, m), 4.16-4.30 (1H, m), 4.78 (2H, td, J=15.95, 2.60 Hz), 6.12 (1H, d, J=9.31 Hz), 6.64 (1H, tt, J=54.30, 3.00 Hz), 7.11 (1H, d, J=3.01 Hz), 7.38 (1H, d, J=2.74 Hz), 7.85 (1H, d, J=8.49 Hz), 8.01 (1H, d, J=8.49 Hz), 10.97 (1H, s); ESIMS found for C₂₂H₂₂[2H₃]F₄N₉ m/z 495.3 (M+1).

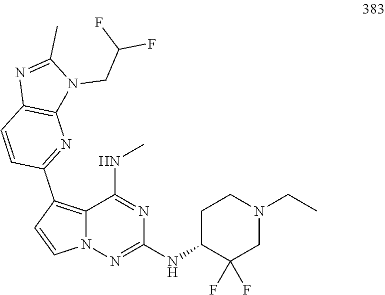 [SMILES: CCN1CC[C@@H](Nc2nc(NC)c3c(-c4ccc5nc(C)n(CC(F)F)c5n4)ccn3n2)C(F)(F)C1]

### (R)-5-(3-(2,2-Difluoroethyl)-2-methyl-3H-imidazo[4,5-b]pyridin-5-yl)-N²-(1-ethyl-3,3-difluoropiperidin-4-yl)-N⁴-methylpyrrolo[2,1-f][1,2,4]triazine-2,4-diamine 383

Off-white solid (19 mg, 0.038 mmol, 59.8% yield). ¹H NMR (499 MHz, DMSO-d₆) δ ppm 1.01 (3H, t, J=7.12 Hz), 1.74 (1H, qd, J=11.96, 3.56 Hz), 1.85 (1H, br dd, J=8.76, 4.11 Hz), 2.16 (1H, br t, J=10.54 Hz), 2.36 (1H, dd, J=27.70, 12.35 Hz), 2.44 (2H, q, J=7.12 Hz), 2.62 (3H, s), 2.86 (1H, br d, J=11.77 Hz), 3.06-3.13 (1H, m), 3.14 (3H, d, J=4.65 Hz), 4.18-4.32 (1H, m), 4.78 (2H, td, J=16.02, 2.74 Hz), 6.12 (1H, d, J=9.31 Hz), 6.64 (1H, tt, J=54.30, 3.00 Hz), 7.11 (1H, d, J=3.01 Hz), 7.38 (1H, d, J=3.01 Hz), 7.85 (1H, d, J=8.76 Hz), 8.01 (1H, d, J=8.49 Hz), 11.00 (1H, q, J=4.56 Hz); ESIMS found for C₂₃H₂₇F₄N₉ m/z 506.3 (M+1).

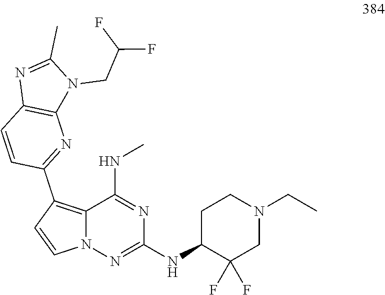 [SMILES: CCN1CC[C@H](Nc2nc(NC)c3c(-c4ccc5nc(C)n(CC(F)F)c5n4)ccn3n2)C(F)(F)C1]

### (S)-5-(3-(2,2-Difluoroethyl)-2-methyl-3H-imidazo[4,5-b]pyridin-5-yl)-N²-(1-ethyl-3,3-difluoropiperidin-4-yl)-N⁴-methylpyrrolo[2,1-f][1,2,4]triazine-2,4-diamine 384

Off-white solid (13 mg, 0.026 mmol, 40.9% yield). ¹H NMR (499 MHz, DMSO-d₆) δ ppm 1.01 (3H, t, J=7.12 Hz), 1.69-1.79 (1H, m), 1.85 (1H, br dd, J=8.90, 4.24 Hz), 2.16 (1H, br t, J=10.81 Hz), 2.36 (1H, dd, J=27.70, 11.75 Hz), 2.44 (2H, q, J=7.03 Hz), 2.62 (3H, s), 2.86 (1H, brd, J=11.50 Hz), 3.06-3.12 (1H, m), 3.14 (3H, d, J=4.65 Hz), 4.17-4.32 (1H, m), 4.78 (2H, td, J=15.95, 2.60 Hz), 6.11 (1H, d, J=9.58 Hz), 6.64 (1H, tt, J=54.30, 3.00 Hz), 7.11 (1H, d, J=3.01 Hz), 7.38 (1H, d, J=2.74 Hz), 7.85 (1H, d, J=8.76 Hz), 8.01 (1H, d, J=8.49 Hz), 10.99 (1H, q, J=4.47 Hz); ESIMS found for C₂₃H₂₇F₄N₉ m/z 506.3 (M+1).

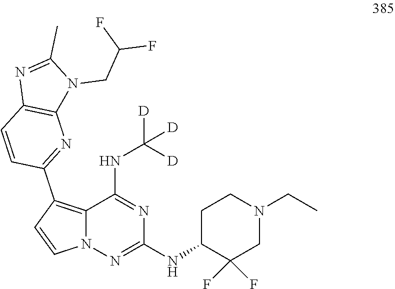 [SMILES: [2H]C([2H])([2H])Nc1nc(N[C@@H]2CCN(CC)CC2(F)F)nn2ccc(-c3ccc4nc(C)n(CC(F)F)c4n3)c12]

### (R)-5-(3-(2,2-Difluoroethyl)-2-methyl-3H-imidazo[4,5-b]pyridin-5-yl)-N²-(1-ethyl-3,3-difluoropiperidin-4-yl)-N⁴-(methyl-d₃)pyrrolo[2,1-f][1,2,4]triazine-2,4-diamine 385

White solid (6 mg, 0.012 mmol, 18.9% yield). ¹H NMR (499 MHz, DMSO-d₆) δ ppm 1.01 (3H, t, J=7.26 Hz), 1.66-1.80 (1H, m), 1.80-1.90 (1H, m), 2.16 (1H, br t, J=10.95 Hz), 2.36 (1H, dd, J=27.45, 12.05 Hz), 2.44 (2H, q, J=6.84 Hz), 2.62 (3H, s), 2.86 (1H, br d, J=11.77 Hz), 3.05-3.17 (1H, m), 4.16-4.33 (1H, m), 4.73-4.84 (2H, m), 6.12 (1H, d, J=9.58 Hz), 6.64 (1H, tt, J=54.18, 3.00 Hz), 7.11 (1H, d, J=3.01 Hz), 7.38 (1H, d, J=2.74 Hz), 7.85 (1H, d, J=8.49 Hz), 8.01 (1H, d, J=8.49 Hz), 10.97 (1H, s); ESIMS found for C₂₃H₂₄[2H₃]F₄N₉ m/z 509.2 (M+1).

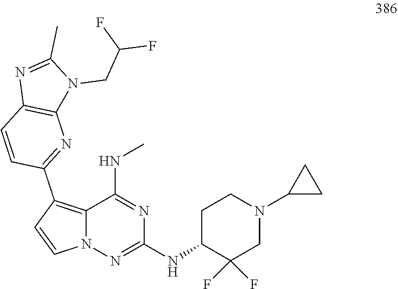 [SMILES: CNc1nc(N[C@@H]2CCN(C3CC3)CC2(F)F)nn2ccc(-c3ccc4nc(C)n(CC(F)F)c4n3)c12]

### (R)—N²-(1-Cyclopropyl-3,3-difluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 386

Pale-yellow semi-solid (2 mg, 0.004 mmol, 18.5% yield). ¹H NMR (499 MHz, DMSO-d₆) δ ppm 0.27-0.33 (1H, m), 0.33-0.39 (1H, m), 0.46 (2H, br d, J=6.30 Hz), 1.64-1.74 (1H, m), 1.76-1.86 (2H, m), 2.45 (1H, br t, J=10.54 Hz), 2.62 (3H, s), 2.63-2.71 (1H, m), 2.94 (1H, brd, J=11.50 Hz), 3.10-3.19 (1H, m), 3.14 (3H, d, J=4.65 Hz), 4.19-4.36 (1H, m), 4.70-4.84 (2H, m), 6.12 (1H, d, J=9.58 Hz), 6.64 (1H, tt, J=54.30, 3.00 Hz), 7.12 (1H, d, J=2.74 Hz), 7.38 (1H, d, J=2.74 Hz), 7.86 (1H, d, J=8.49 Hz), 8.01 (1H, d, J=8.49 Hz), 11.00 (1H, q, J=4.38 Hz); ESIMS found for C₂₄H₂₇F₄N₉ m/z 518.3 (M+1).

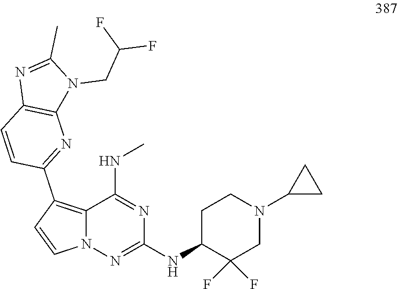 [SMILES: CNc1nc(N[C@H]2CCN(C3CC3)CC2(F)F)nn2ccc(-c3ccc4nc(C)n(CC(F)F)c4n3)c12]

### (S)—N²-(1-Cyclopropyl-3,3-difluoropiperidin-4-yl)-5-(3-(2,2-difluoroethyl)-2-methyl-3H-imidazo[4,5-b]pyridin-5-yl)-N⁴-methylpyrrolo[2,1-f][1,2,4]triazine-2,4-diamine 387

Pale-yellow semi-solid (1.3 mg, 0.003 mmol, 12.0% yield). ¹H NMR (499 MHz, DMSO-d₆) δ ppm 0.27-0.33 (1H, m), 0.33-0.39 (1H, m), 0.46 (2H, br d, J=6.30 Hz), 1.64-1.74 (1H, m), 1.76-1.87 (1H, m), 1.97-2.04 (1H, m), 2.45 (1H, br t, J=10.40 Hz), 2.62 (3H, s), 2.63-2.72 (1H, m), 2.94 (1H, br d, J=11.50 Hz), 3.10-3.18 (1H, m), 3.14 (3H, d, J=4.65 Hz), 4.19-4.36 (1H, m), 4.71-4.86 (2H, m), 6.12 (1H, d, J=9.31 Hz), 6.64 (1H, tt, J=54.30, 3.00 Hz), 7.12 (1H, d, J=3.01 Hz), 7.38 (1H, d, J=3.01 Hz), 7.86 (1H, d, J=8.49 Hz), 8.01 (1H, d, J=8.49 Hz), 10.97-11.03 (1H, m); ESIMS found for C₂₄H₂₇F₄N₉ m/z 518.3 (M+1).

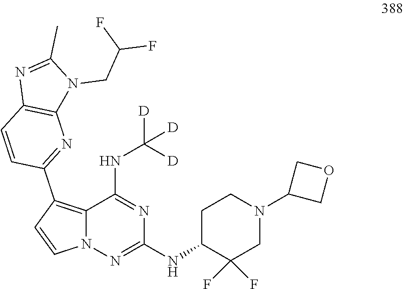 [SMILES: [2H]C([2H])([2H])Nc1nc(N[C@@H]2CCN(C3COC3)CC2(F)F)nn2ccc(-c3ccc4nc(C)n(CC(F)F)c4n3)c12]

### (R)—N²-(3,3-Difluoro-1-(oxetan-3-yl)piperidin-4-yl)-5-(3-(2,2-difluoroethyl)-2-methyl-3H-imidazo[4,5-b]pyridin-5-yl)-N⁴-(methyl-d₃)pyrrolo[2,1-f][1,2,4]triazine-2,4-diamine 388

White solid (12 mg, 0.022 mmol, 43.0% yield). ¹H NMR (499 MHz, DMSO-d₆) δ ppm 1.71-1.83 (1H, m), 1.76-1.76 (1H, m), 1.88 (1H, br dd, J=9.03, 4.38 Hz), 2.14 (1H, br t, J=10.54 Hz), 2.38 (1H, dd, J=26.05, 11.50 Hz), 2.74 (1H, br d, J=11.23 Hz), 2.95-3.06 (1H, m), 3.60 (1H, quin, J=6.30 Hz), 4.22-4.37 (1H, m), 4.44 (2H, dt, J=14.37, 6.23 Hz), 4.55 (2H, td, J=6.57, 3.29 Hz), 4.78 (2H, td, J=15.88, 2.19 Hz), 6.20 (1H, d, J=9.58 Hz), 6.64 (1H, tt, J=54.30, 3.00 Hz), 7.11 (1H, d, J=3.01 Hz), 7.37 (1H, d, J=3.01 Hz), 7.85 (1H, d, J=8.76 Hz), 8.01 (1H, d, J=8.49 Hz), 10.98 (1H, s); ESIMS found for C₂₄H₂₄[2H₃]F₄N₉O m/z 537.3 (M+1).

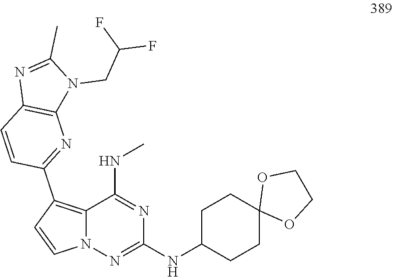 [SMILES: CNc1nc(NC2CCC3(CC2)OCCO3)nn2ccc(-c3ccc4nc(C)n(CC(F)F)c4n3)c12]

### 5-(3-(2,2-Difluoroethyl)-2-methyl-3H-imidazo[4,5-b]pyridin-5-yl)-N⁴-methyl-N²-(1,4-dioxaspiro[4.5]decan-8-yl)pyrrolo[2,1-f][1,2,4]triazine-2,4-diamine 389

Off-white solid (72 mg, 0.144 mmol, 72.7% yield). ¹H NMR (499 MHz, DMSO-d₆) δ ppm 1.49-1.63 (4H, m), 1.72 (2H, br dd, J=7.67, 3.83 Hz), 1.83-1.94 (2H, m), 2.62 (3H, s), 3.11 (3H, d, J=4.65 Hz), 3.64 (1H, br d, J=3.56 Hz), 3.83-3.90 (4H, m), 4.77 (2H, td, J=15.81, 2.60 Hz), 5.99 (1H, d, J=7.94 Hz), 6.63 (1H, tt, J=54.30, 3.00 Hz), 7.08 (1H, d, J=3.01 Hz), 7.36 (1H, d, J=3.01 Hz), 7.84 (1H, d, J=8.76 Hz), 8.00 (1H, d, J=8.49 Hz), 10.89 (1H, q, J=4.56 Hz); ESIMS found for C₂₄H₂₈F₂N₈O₂ m/z 499.3 (M+1).

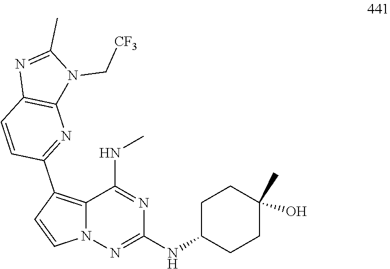 [SMILES: CNc1nc(N[C@H]2CC[C@@](C)(O)CC2)nn2ccc(-c3ccc4nc(C)n(CC(F)(F)F)c4n3)c12]

### (1s,4s)-1-Methyl-4-((5-(2-methyl-3-(2,2,2-trifluoroethyl)-3H-imidazo[4,5-b]pyridin-5-yl)-4-(methylamino)pyrrolo[2,1-f][1,2,4]triazin-2-yl)amino)cyclohexan-1-ol 441

Beige solid (7 mg, 0.014 mmol, 11.3% yield). ¹H NMR (499 MHz, DMSO-d₆) δ ppm 1.12 (3H, s), 1.35 (2H, td, J=13.21, 3.97 Hz), 1.51-1.64 (4H, m), 1.64-1.73 (2H, m), 2.64 (3H, s), 3.10 (3H, d, J=4.65 Hz), 3.43-3.56 (1H, m), 3.97 (1H, s), 5.29 (2H, q, J=9.31 Hz), 5.86 (1H, d, J=7.94 Hz), 7.10 (1H, d, J=3.01 Hz), 7.35 (1H, d, J=3.01 Hz), 7.89 (1H, d, J=8.76 Hz), 8.03 (1H, d, J=8.49 Hz), 10.78 (1H, q, J=4.56 Hz); ESIMS found for C₂₃H₂₇F₃N₈O m/z 489.3 (M+1).

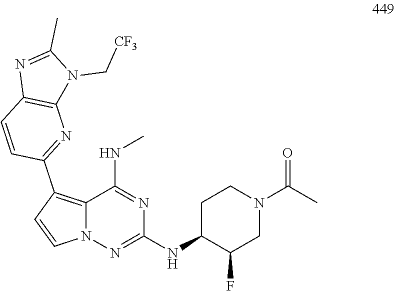 [SMILES: CNc1nc(N[C@H]2CCN(C(C)=O)C[C@H]2F)nn2ccc(-c3ccc4nc(C)n(CC(F)(F)F)c4n3)c12]

### 1-((3R,4S)-3-Fluoro-4-((5-(2-methyl-3-(2,2,2-trifluoroethyl)-3H-imidazo[4,5-b]pyridin-5-yl)-4-(methylamino)pyrrolo[2,1-f][1,2,4]triazin-2-yl)amino)piperidin-1-yl)ethan-1-one 449

White solid (10 mg, 0.019 mmol, 38.6% yield). ¹H NMR (499 MHz, DMSO-d₆) δ ppm 1.60-1.72 (1H, m), 1.74-1.87 (1H, m), 1.98-2.07 (3H, m), 2.66-2.96 (1H, m), 3.12 (3H, d, J=4.93 Hz), 3.89 (1H, br d, J=11.50 Hz), 3.93-4.06 (1H, m), 4.07-4.17 (1H, m), 4.40-4.75 (1H, m), 4.97 (1H, d, J=49.35 Hz), 5.30 (2H, q, J=9.13 Hz), 6.12 (1H, dd, J=7.94, 3.01 Hz), 7.14 (1H, d, J=3.29 Hz), 7.38 (1H, d, J=3.29 Hz), 7.91 (1H, d, J=8.76 Hz), 8.04 (1H, d, J=8.76 Hz), 10.89 (1H, q, J=4.93 Hz); ESIMS found for C₂₃H₂₅F₄N₉O m/z 520.25 (M+1).

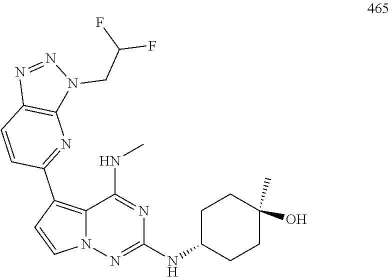 [SMILES: CNc1nc(N[C@H]2CC[C@](C)(O)CC2)nn2ccc(-c3ccc4nnn(CC(F)F)c4n3)c12]

### (1r,4r)-4-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-1-methylcyclohexan-1-ol 465

Fluffy orange solid (19 mg, 0.042 mmol, 64.3% yield). ¹H NMR (499 MHz, DMSO-d₆) δ ppm 1.14 (3H, s), 1.34-1.49 (4H, m), 1.53-1.65 (2H, m), 1.79-1.91 (2H, m), 3.12 (3H, d, J=4.65 Hz), 3.63 (1H, br s), 4.22 (1H, s), 5.25 (2H, td, J=15.95, 2.60 Hz), 6.04 (1H, d, J=8.21 Hz), 6.72 (1H, tt, J=54.30, 3.00 Hz), 7.31 (1H, d, J=3.29 Hz), 7.44 (1H, d, J=3.01 Hz), 8.10 (1H, d, J=9.04 Hz), 8.54 (1H, d, J=9.03 Hz), 10.85 (1H, q, J=4.56 Hz); ESIMS found for C₂₁H₂₅F₂N₉O m/z 458.2 (M+1).

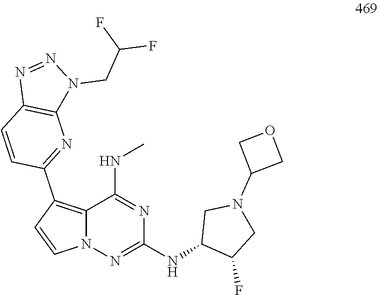 [SMILES: CNc1nc(N[C@@H]2CN(C3COC3)C[C@@H]2F)nn2ccc(-c3ccc4nnn(CC(F)F)c4n3)c12]

### 5-(3-(2,2-Difluoroethyl)-3H-[1,2,3]triazolo[4,5-b]pyridin-5-yl)-N²-((3R,4S)-4-fluoro-1-(oxetan-3-yl)pyrrolidin-3-yl)-N⁴-methylpyrrolo[2,1-f][1,2,4]triazine-2,4-diamine 469

Yellow solid (32 mg, 0.066 mmol, 63.0% yield). ¹H NMR (499 MHz, DMSO-d₆) δ ppm 2.64 (1H, t, J=9.31 Hz), 2.75 (1H, ddd, J=30.20, 12.05, 1.10 Hz), 3.00 (1H, t, J=8.21 Hz), 3.14 (3H, d, J=4.93 Hz), 3.18 (1H, ddd, J=32.90, 12.05, 4.38 Hz), 3.78 (1H, quin, J=6.16 Hz), 4.24-4.41 (1H, m), 4.47 (2H, t, J=5.75 Hz), 4.59 (2H, t, J=6.57 Hz), 5.13-5.27 (1H, m), 5.27 (2H, td, J=16.45, 2.75 Hz), 6.32 (1H, d, J=7.67 Hz), 6.72 (1H, tt, J=54.30, 3.00 Hz), 7.35 (1H, d, J=2.74 Hz), 7.43-7.48 (1H, m), 8.12 (1H, d, J=9.31 Hz), 8.56 (1H, d, J=8.76 Hz), 10.95 (1H, q, J=4.38 Hz); ESIMS found for C₂₁H₂₃F₃N₁₀O m/z 489.2 (M+1).

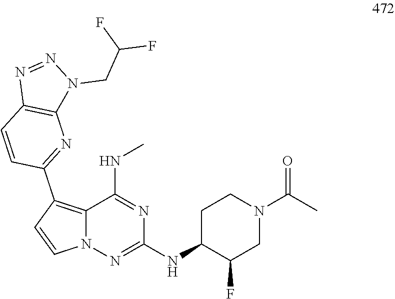 [SMILES: CNc1nc(N[C@H]2CCN(C(C)=O)C[C@H]2F)nn2ccc(-c3ccc4nnn(CC(F)F)c4n3)c12]

### 1-((3R,4S)-4-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-3-fluoropiperidin-1-yl)ethan-1-one 472

Yellow solid (12 mg, 0.025 mmol, 49.2% yield). ¹H NMR (499 MHz, DMSO-d₆) δ ppm 1.61-1.72 (1H, m), 1.72-1.89 (1H, m), 1.97-2.07 (3H, m), 2.80-2.96 (1H, m), 3.14 (3H, d, J=4.38 Hz), 3.89 (1H, br d, J=14.24 Hz), 3.94-4.06 (1H, m), 4.07-4.16 (1H, m), 4.40-4.75 (1H, m), 4.97 (1H, d, J=48.80 Hz), 5.27 (2H, td, J=16.02, 2.46 Hz), 6.28 (1H, d, J=7.67 Hz), 6.72 (1H, tt, J=53.70, 3.00 Hz), 7.35 (1H, d, J=2.74 Hz), 7.44 (1H, d, J=3.29 Hz), 8.12 (1H, d, J=9.31 Hz), 8.56 (1H, d, J=8.76 Hz), 10.94 (1H, q, J=4.38 Hz); ESIMS found for C₂₁H₂₃F₃N₁₀O m/z 489.2 (M+1).

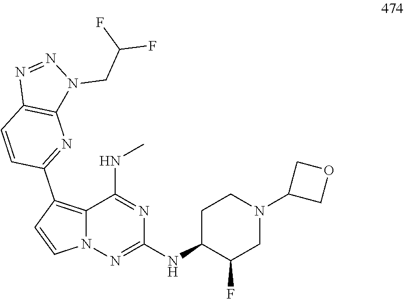 [SMILES: CNc1nc(N[C@H]2CCN(C3COC3)C[C@H]2F)nn2ccc(-c3ccc4nnn(CC(F)F)c4n3)c12]

### 5-(3-(2,2-Difluoroethyl)-3H-[1,2,3]triazolo[4,5-b]pyridin-5-yl)-N²-((3R,4S)-3-fluoro-1-(oxetan-3-yl)piperidin-4-yl)-N⁴-methylpyrrolo[2,1-f][1,2,4]triazine-2,4-diamine 474

Fluffy yellow solid (16 mg, 0.032 mmol, 56.5% yield). ¹H NMR (499 MHz, DMSO-d₆) δ ppm 1.71 (1H, br d, J=10.13 Hz), 1.88 (1H, qd, J=12.18, 3.42 Hz), 1.96-2.04 (1H, m), 2.13 (1H, dd, J=36.75, 12.32 Hz), 2.75 (1H, br d, J=9.58 Hz), 2.98 (1H, br t, J=10.40 Hz), 3.13 (3H, d, J=4.65 Hz), 3.49 (1H, quin, J=6.30 Hz), 3.74-3.92 (1H, m), 4.40 (1H, t, J=6.16 Hz), 4.46 (1H, t, J=6.16 Hz), 4.54 (2H, td, J=6.50, 3.15 Hz), 4.90 (1H, d, J=49.35 Hz), 5.26 (2H, td, J=16.08, 2.60 Hz), 6.17 (1H, d, J=8.21 Hz), 6.72 (1H, tt, J=54.05, 3.00 Hz), 7.34 (1H, d, J=3.01 Hz), 7.44 (1H, d, J=2.74 Hz), 8.11 (1H, d, J=9.31 Hz), 8.55 (1H, d, J=9.03 Hz), 10.93 (1H, q, J=4.56 Hz); ESIMS found for C₂₂H₂₅F₃N₁₀O m/z 503.2 (M+1).

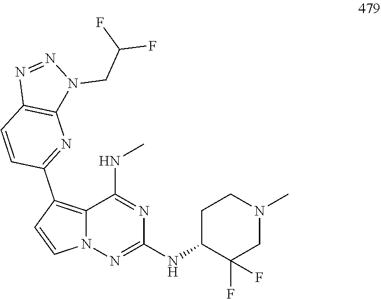 [SMILES: CNc1nc(N[C@@H]2CCN(C)CC2(F)F)nn2ccc(-c3ccc4nnn(CC(F)F)c4n3)c12]

### (R)—N²-(3,3-Difluoro-1-methylpiperidin-4-yl)-5-(3-(2,2-difluoroethyl)-3H-[1,2,3]triazolo[4,5-b]pyridin-5-yl)-N4-methylpyrrolo[2,1-f][1,2,4]triazine-2,4-diamine 479

Fluffy yellow solid (11 mg, 0.023 mmol, 47.5% yield). ¹H NMR (499 MHz, DMSO-d₆) δ ppm 1.72-1.90 (2H, m), 2.09-2.20 (1H, m), 2.25 (3H, s), 2.36 (1H, dd, J=28.00, 11.77 Hz), 2.78 (1H, brd, J=10.13 Hz), 2.97-3.08 (1H, m), 3.14 (3H, d, J=4.65 Hz), 4.15-4.32 (1H, m), 5.27 (2H, td, J=16.02, 2.46 Hz), 6.33 (1H, d, J=9.58 Hz), 6.72 (1H, tt, J=54.00, 3.00 Hz), 7.35 (1H, d, J=3.29 Hz), 7.45 (1H, d, J=3.01 Hz), 8.12 (1H, d, J=9.03 Hz), 8.56 (1H, d, J=9.04 Hz), 10.95 (1H, q, J=4.65 Hz); ESIMS found for C₂₀H₂₂F₄N₁₀ m/z 479.2 (M+1).

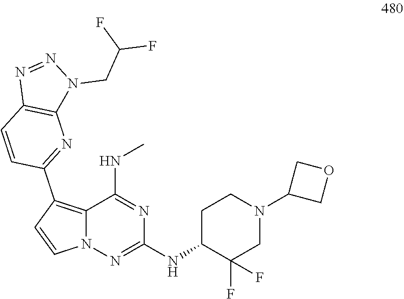 [SMILES: CNc1nc(N[C@@H]2CCN(C3COC3)CC2(F)F)nn2ccc(-c3ccc4nnn(CC(F)F)c4n3)c12]

### (R)—N²-(3,3-Difluoro-1-(oxetan-3-yl)piperidin-4-yl)-5-(3-(2,2-difluoroethyl)-3H-[1,2,3]triazolo[4,5-b]pyridin-5-yl)-N⁴-methylpyrrolo[2,1-f][1,2,4]triazine-2,4-diamine 480

Fluffy yellow solid (11 mg, 0.021 mmol, 43.8% yield). ¹H NMR (499 MHz, DMSO-d₆) δ ppm 1.73-1.83 (1H, m), 1.84-1.91 (1H, m), 2.14 (1H, br t, J=10.54 Hz), 2.38 (1H, dd, J=25.50, 12.05 Hz), 2.75 (1H, br d, J=12.59 Hz), 2.95-3.07 (1H, m), 3.15 (3H, d, J=4.65 Hz), 3.60 (1H, quin, J=6.30 Hz), 4.21-4.37 (1H, m), 4.44 (2H, dt, J=14.51, 6.16 Hz), 4.55 (2H, td, J=6.64, 3.42 Hz), 5.27 (2H, td, J=16.02, 2.46 Hz), 6.41 (1H, d, J=9.58 Hz), 6.72 (1H, tt, J=54.00, 3.00 Hz), 7.35 (1H, d, J=3.29 Hz), 7.44 (1H, d, J=3.01 Hz), 8.12 (1H, d, J=9.04 Hz), 8.56 (1H, d, J=9.03 Hz), 10.96 (1H, q, J=4.65 Hz); ESIMS found for C₂₂H₂₄F₄N₁₀O m/z 521.2 (M+1).

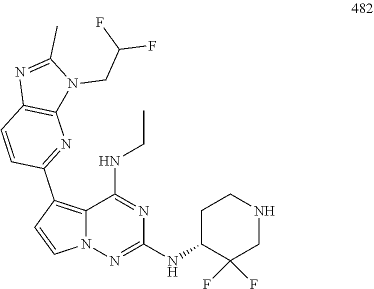 [SMILES: CCNc1nc(N[C@@H]2CCNCC2(F)F)nn2ccc(-c3ccc4nc(C)n(CC(F)F)c4n3)c12]

### (R)-5-(3-(2,2-Difluoroethyl)-2-methyl-3H-imidazo[4,5-b]pyridin-5-yl)-N²-(3,3-difluoropiperidin-4-yl)-N⁴-ethylpyrrolo[2,1-f][1,2,4]triazine-2,4-diamine 482

White solid (100 mg, 0.204 mmol, 43.9% yield). ¹H NMR (499 MHz, DMSO-d₆) δ ppm 1.24 (3H, t, J=6.98 Hz), 1.65 (1H, qd, J=11.96, 3.56 Hz), 1.78-1.88 (1H, m), 2.54-2.61 (1H, m), 2.63 (3H, s), 2.77 (1H, dd, J=29.35, 13.75 Hz), 2.92 (1H, br d, J=12.87 Hz), 3.05-3.17 (1H, m), 3.67 (2H, quin, J=6.78 Hz), 4.25-4.40 (1H, m), 4.75 (2H, td, J=15.74, 2.74 Hz), 6.13 (1H, d, J=9.58 Hz), 6.62 (1H, tt, J=54.30, 3.00 Hz), 7.09 (1H, d, J=3.01 Hz), 7.38 (1H, d, J=2.74 Hz), 7.84 (1H, d, J=8.76 Hz), 8.01 (1H, d, J=8.49 Hz), 10.93 (1H, t, J=6.16 Hz); ESIMS found for C₂₂H₂₅F₄N₉ m/z 492.25 (M+1).

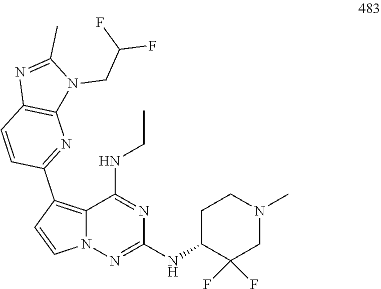 [SMILES: CCNc1nc(N[C@@H]2CCN(C)CC2(F)F)nn2ccc(-c3ccc4nc(C)n(CC(F)F)c4n3)c12]

### (R)—N²-(3,3-difluoro-1-methylpiperidin-4-yl)-5-(3-(2,2-difluoroethyl)-2-methyl-3H-imidazo[4,5-b]pyridin-5-yl)-N⁴-ethylpyrrolo[2,1-f][1,2,4]triazine-2,4-diamine 483

Off-white solid (2 mg, 0.004 mmol, 3.9% yield). ¹H NMR (499 MHz, DMSO-d₆) δ ppm 1.24 (3H, t, J=6.98 Hz), 1.72-1.81 (1H, m), 1.81-1.88 (1H, m), 2.10-2.19 (1H, m), 2.25 (3H, s), 2.29-2.40 (1H, m), 2.63 (3H, s), 2.78 (1H, br d, J=11.23 Hz), 3.00-3.11 (1H, m), 3.66 (2H, quin, J=7.05 Hz), 4.15-4.31 (1H, m), 4.69-4.81 (2H, m), 6.12 (1H, d, J=9.31 Hz), 6.62 (1H, tt, J=54.30, 3.00 Hz), 7.09 (1H, d, J=3.01 Hz), 7.38 (1H, d, J=2.74 Hz), 7.84 (1H, d, J=8.76 Hz), 8.01 (1H, d, J=8.49 Hz), 10.94 (1H, t, J=6.16 Hz); ESIMS found for C₂₃H₂₇F₄N₉ m/z 506.2 (M+1).

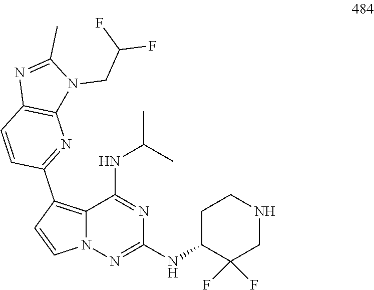 [SMILES: Cc1nc2ccc(-c3ccn4nc(N[C@@H]5CCNCC5(F)F)nc(NC(C)C)c34)nc2n1CC(F)F]

### (R)-5-(3-(2,2-Difluoroethyl)-2-methyl-3H-imidazo[4,5-b]pyridin-5-yl)-N²-(3,3-difluoropiperidin-4-yl)-N⁴-isopropylpyrrolo[2,1-f][1,2,4]triazine-2,4-diamine 484

White solid (118 mg, 0.233 mmol, 81.2% yield). ¹H NMR (499 MHz, DMSO-d₆) δ ppm 1.32 (6H, dd, J=6.57, 1.64 Hz), 1.59-1.73 (1H, m), 1.79-1.89 (1H, m), 2.57 (1H, br t, J=11.77 Hz), 2.63 (3H, s), 2.77 (1H, dd, J=28.80, 13.69 Hz), 2.92 (1H, br d, J=12.32 Hz), 3.06-3.16 (1H, m), 4.24-4.40 (1H, m), 4.51-4.63 (1H, m), 4.82 (2H, td, J=15.19, 3.29 Hz), 6.16 (1H, d, J=9.31 Hz), 6.55 (1H, tt, J=54.30, 3.00 Hz), 6.94 (1H, d, J=3.01 Hz), 7.40 (1H, d, J=2.74 Hz), 7.73 (1H, d, J=8.49 Hz), 8.03 (1H, d, J=8.21 Hz), 9.45 (1H, d, J=8.21 Hz); ESIMS found for C₂₃H₂₇F₄N₉ m/z 506.3 (M+1).

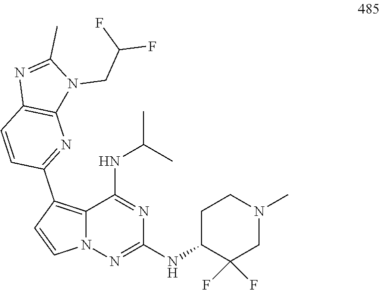 [SMILES: Cc1nc2ccc(-c3ccn4nc(N[C@@H]5CCN(C)CC5(F)F)nc(NC(C)C)c34)nc2n1CC(F)F]

### (R)—N²-(3,3-Difluoro-1-methylpiperidin-4-yl)-5-(3-(2,2-difluoroethyl)-2-methyl-3H-imidazo[4,5-b]pyridin-5-yl)-N⁴-isopropylpyrrolo[2,1-f][1,2,4]triazine-2,4-diamine 485

Off-white solid (31 mg, 0.060 mmol, 60.3% yield). ¹H NMR (499 MHz, DMSO-d₆) δ ppm 1.32 (6H, dd, J=6.71, 1.51 Hz), 1.74-1.81 (1H, m), 1.81-1.87 (1H, m), 2.15 (1H, br t, J=10.54 Hz), 2.25 (3H, s), 2.29-2.39 (1H, m), 2.63 (3H, s), 2.73-2.82 (1H, m), 2.98-3.10 (1H, m), 4.15-4.31 (1H, m), 4.51-4.59 (1H, m), 4.82 (2H, td, J=15.19, 3.29 Hz), 6.15 (1H, d, J=9.31 Hz), 6.55 (1H, tt, J=54.30, 3.00 Hz), 6.94 (1H, d, J=2.74 Hz), 7.40 (1H, d, J=2.74 Hz), 7.73 (1H, d, J=8.49 Hz), 8.03 (1H, d, J=8.49 Hz), 9.46 (1H, d, J=8.21 Hz); ESIMS found for C₂₄H₂₉F₄N₉ m/z 520.3 (M+1).

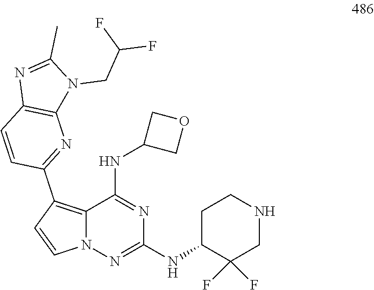 [SMILES: Cc1nc2ccc(-c3ccn4nc(N[C@@H]5CCNCC5(F)F)nc(NC5COC5)c34)nc2n1CC(F)F]

### (R)-5-(3-(2,2-Difluoroethyl)-2-methyl-3H-imidazo[4,5-b]pyridin-5-yl)-N²-(3,3-difluoropiperidin-4-yl)-N⁴-(oxetan-3-yl)pyrrolo[2,1-f][1,2,4]triazine-2,4-diamine 486

White solid (18 mg, 0.035 mmol, 32.5% yield). ¹H NMR (499 MHz, DMSO-d₆) δ ppm 1.67-1.81 (1H, m), 1.87 (1H, br d, J=11.23 Hz), 2.55-2.63 (1H, m), 2.59 (3H, s), 2.72-2.89 (1H, m), 2.94 (1H, br d, J=12.05 Hz), 3.07-3.20 (1H, m), 3.41-3.56 (1H, m), 3.57-3.68 (1H, m), 3.70-3.97 (1H, m), 3.98-4.19 (1H, m), 4.23-4.42 (2H, m), 4.75 (2H, brt, J=13.96 Hz), 4.82-4.95 (1H, m), 6.16-6.35 (1H, m), 6.42-6.68 (1H, m), 7.02 (1H, br s), 7.22 (1H, br d, J=1.92 Hz), 7.87 (1H, br s), 8.82-8.97 (1H, m); ESIMS found for C₂₃H₂₅F₄N₉O m/z 520.2 (M+1).

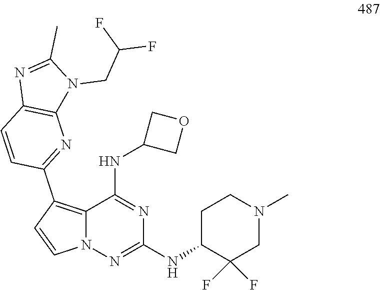 [SMILES: Cc1nc2ccc(-c3ccn4nc(N[C@@H]5CCN(C)CC5(F)F)nc(NC5COC5)c34)nc2n1CC(F)F]

### (R)—N2-(3,3-Difluoro-1-methylpiperidin-4-yl)-5-(3-(2,2-difluoroethyl)-2-methyl-3H-imidazo[4,5-b]pyridin-5-yl)-N4-(oxetan-3-yl)pyrrolo[2,1-f][1,2,4]triazine-2,4-diamine 487

Tan solid (4.1 mg, 0.008 mmol, 8.7% yield). ¹H NMR (499 MHz, DMSO-d₆) δ ppm 1.72-1.85 (2H, m), 2.15 (1H, br t, J=11.09 Hz), 2.25 (3H, s), 2.31-2.42 (1H, m), 2.62 (3H, s), 2.77 (1H, br d, J=11.50 Hz), 2.99-3.09 (1H, m), 4.16-4.33 (1H, m), 4.67 (1H, t, J=6.30 Hz), 4.70 (1H, br t, J=6.43 Hz), 4.78 (2H, br t, J=14.92 Hz), 4.94 (2H, t, J=6.98 Hz), 5.20-5.29 (1H, m), 6.28 (1H, br d, J=9.31 Hz), 6.53 (1H, tt, J=54.30, 3.00 Hz), 7.05 (1H, d, J=3.01 Hz), 7.44 (1H, d, J=2.74 Hz), 7.81 (1H, d, J=8.49 Hz), 8.04 (1H, d, J=8.49 Hz), 10.50 (1H, d, J=6.02 Hz); ESIMS found for C₂₄H₂₇F₄N₉O m/z 534.2 (M+1).

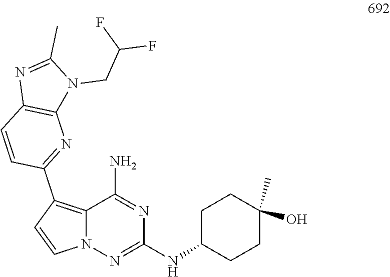 [SMILES: Cc1nc2ccc(-c3ccn4nc(N[C@H]5CC[C@](C)(O)CC5)nc(N)c34)nc2n1CC(F)F]

(1r,4r)-4-((4-Amino-5-(3-(2,2-difluoroethyl)-2-methyl-3H-imidazo[4,5-b]pyridin-5-yl)pyrrolo[2,1-f][1,2,4]triazin-2-yl)amino)-1-methylcyclohexan-1-ol 692.

White solid (20 mg, 0.044 mmol, 74.9% yield). ¹H NMR (499 MHz, DMSO-d₆) δ ppm 1.14 (3H, s), 1.33-1.46 (4H, m), 1.51-1.62 (2H, m), 1.78-1.87 (2H, m), 2.60 (3H, s), 3.61 (1H, br dd, J=8.21, 3.83 Hz), 4.21 (1H, s), 4.70 (2H, td, J=16.45, 1.65 Hz), 5.74 (1H, d, J=8.21 Hz), 6.46 (1H, tt, J=54.00, 2.75 Hz), 7.07 (1H, d, J=2.74 Hz), 7.38 (1H, d, J=2.74 Hz), 7.42 (1H, br s), 7.80 (1H, d, J=8.76 Hz), 7.99 (1H, d, J=8.21 Hz), 10.52 (1H, br s); ESIMS found for C₂₂H₂₆F₂N₈O m/z 457.2 (M+1).

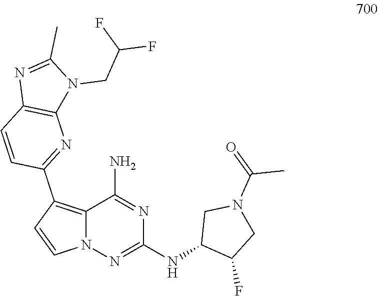 [SMILES: CC(=O)N1C[C@H](F)[C@H](Nc2nc(N)c3c(-c4ccc5nc(C)n(CC(F)F)c5n4)ccn3n2)C1]

### 1-((3R,4S)-3-((4-Amino-5-(3-(2,2-difluoroethyl)-2-methyl-3H-imidazo[4,5-b]pyridin-5-yl)pyrrolo[2,1-f][1,2,4]triazin-2-yl)amino)-4-fluoropyrrolidin-1-yl)ethan-1-one 700

White solid (20 mg, 0.042 mmol, 34.8% yield). ¹H NMR (499 MHz, DMSO-d₆) δ ppm 1.92-2.00 (3H, m), 2.60 (3H, s), 3.41-3.58 (1H, m), 3.62-3.73 (1H, m), 3.75-3.86 (1H, m), 3.91 (1H, t, J=9.03 Hz), 4.26-4.56 (1H, m), 4.71 (2H, td, J=16.45, 1.65 Hz), 5.20-5.43 (1H, m), 6.33 (1H, dd, J=11.77, 7.67 Hz), 6.47 (1H, tt, J=54.30, 2.20 Hz), 7.13 (1H, dd, J=3.01, 1.92 Hz), 7.41 (1H, dd, J=4.24, 2.87 Hz), 7.58-7.68 (1H, m), 7.83 (1H, d, J=8.76 Hz), 8.01 (1H, d, J=8.49 Hz), 10.67 (1H, br s); ESIMS found for C₂₁H₂₂F₃N₉O m/z 474.2 (M+1).

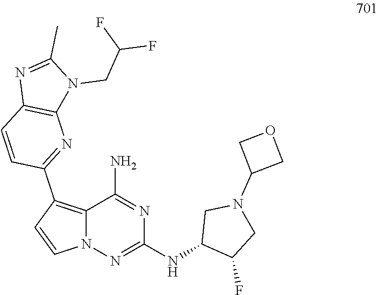 [SMILES: Cc1nc2ccc(-c3ccn4nc(N[C@@H]5CN(C6COC6)C[C@@H]5F)nc(N)c34)nc2n1CC(F)F]

### 5-(3-(2,2-Difluoroethyl)-2-methyl-3H-imidazo[4,5-b]pyridin-5-yl)-N²-((3R,4S)-4-fluoro-1-(oxetan-3-yl)pyrrolidin-3-yl)pyrrolo[2,1-f][1,2,4]triazine-2,4-diamine 701

White solid (29 mg, 0.060 mmol, 46.3% yield). ¹H NMR (499 MHz, DMSO-d₆) δ ppm 2.60 (3H, s), 2.60-2.64 (1H, m), 2.76 (1H, ddd, J=30.15, 12.35, 1.35 Hz), 2.99 (1H, t, J=8.21 Hz), 3.15 (1H, ddd, J=33.75, 12.10, 4.40 Hz), 3.78 (1H, quin, J=6.23 Hz), 4.22-4.37 (1H, m), 4.46 (2H, t, J=6.02 Hz), 4.59 (2H, t, J=6.57 Hz), 4.71 (2H, td, J=16.75, 1.90 Hz), 5.19 (1H, dtd, J=55.70, 4.60, 4.60, 1.35 Hz), 6.02 (1H, d, J=7.94 Hz), 6.47 (1H, tt, J=54.40, 2.20 Hz), 7.11 (1H, d, J=3.01 Hz), 7.41 (1H, d, J=2.74 Hz), 7.61 (1H, br s), 7.82 (1H, d, J=8.49 Hz), 8.00 (1H, d, J=8.49 Hz), 10.62 (1H, br s); ESIMS found for C₂₂H₂₄F₃N₉O m/z 488.2 (M+1).

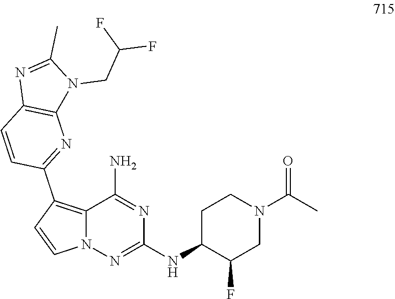 [SMILES: CC(=O)N1CC[C@H](Nc2nc(N)c3c(-c4ccc5nc(C)n(CC(F)F)c5n4)ccn3n2)[C@H](F)C1]

### 1-((3R,4S)-4-((4-Amino-5-(3-(2,2-difluoroethyl)-2-methyl-3H-imidazo[4,5-b]pyridin-5-yl)pyrrolo[2,1-f][1,2,4]triazin-2-yl)amino)-3-fluoropiperidin-1-yl)ethan-1-one 715

Fluffy pale-yellow solid (8 mg, 0.016 mmol, 26.8% yield). ¹H NMR (499 MHz, DMSO-d₆) δ ppm 1.57-1.70 (1H, m), 1.73-1.84 (1H, m), 1.97-2.06 (3H, m), 2.60 (3H, s), 2.67 (1H, td, J=12.80, 3.42 Hz), 2.86 (1H, dd, J=40.05, 13.96 Hz), 3.83-3.98 (1H, m), 4.00-4.17 (1H, m), 4.38-4.48 (1H, m), 4.66-4.79 (2H, m), 4.95 (1H, d, J=49.90 Hz), 5.96 (1H, dd, J=8.21, 5.75 Hz), 6.47 (1H, tt, J=54.00, 2.75 Hz), 7.11 (1H, d, J=3.01 Hz), 7.39 (1H, d, J=3.01 Hz), 7.56 (1H, br s), 7.82 (1H, d, J=8.76 Hz), 8.00 (1H, d, J=8.49 Hz), 10.63 (1H, br s); ESIMS found for C₂₂H₂₄F₃N₉O m/z 244.65 (M/2+1).

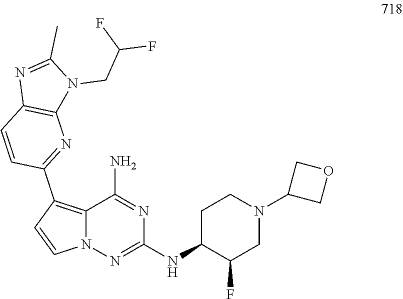 [SMILES: Cc1nc2ccc(-c3ccn4nc(N[C@H]5CCN(C6COC6)C[C@H]5F)nc(N)c34)nc2n1CC(F)F]

### 5-(3-(2,2-Difluoroethyl)-2-methyl-3H-imidazo[4,5-b]pyridin-5-yl)-N²-((3R,4S)-3-fluoro-1-(oxetan-3-yl)piperidin-4-yl)pyrrolo[2,1-f][1,2,4]triazine-2,4-diamine 718

Fluffy pale-yellow solid (3 mg, 0.006 mmol, 14.6% yield). ¹H NMR (499 MHz, DMSO-d₆) δ ppm 1.64-1.75 (1H, m), 1.85 (1H, qd, J=12.05, 3.56 Hz), 1.94-2.02 (1H, m), 2.12 (1H, dd, J=37.00, 12.59 Hz), 2.60 (3H, s), 2.69-2.79 (1H, m), 2.97 (1H, br t, J=10.81 Hz), 3.48 (1H, dt, J=12.66, 6.40 Hz), 3.73-3.91 (1H, m), 4.39 (1H, t, J=6.16 Hz), 4.45 (1H, t, J=6.16 Hz), 4.54 (2H, td, J=6.43, 2.74 Hz), 4.64-4.76 (2H, m), 4.88 (1H, d, J=49.40 Hz), 5.83 (1H, d, J=8.21 Hz), 6.46 (1H, tt, J=54.00, 2.75 Hz), 7.10 (1H, d, J=3.01 Hz), 7.38 (1H, d, J=2.74 Hz), 7.55 (1H, br s), 7.81 (1H, d, J=8.49 Hz), 8.00 (1H, d, J=8.49 Hz), 10.61 (1H, br s); ESIMS found for C₂₃H₂₆F₃N₉O m/z 251.65 (M/2+1).

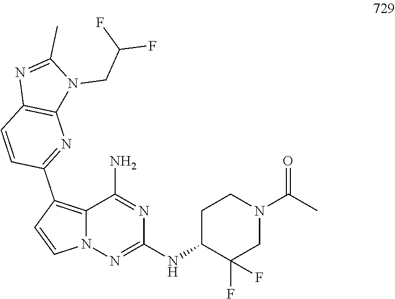 [SMILES: CC(=O)N1CC[C@@H](Nc2nc(N)c3c(-c4ccc5nc(C)n(CC(F)F)c5n4)ccn3n2)C(F)(F)C1]

### (R)-1-(4-((4-Amino-5-(3-(2,2-difluoroethyl)-2-methyl-3H-imidazo[4,5-b]pyridin-5-yl)pyrrolo[2,1-f][1,2,4]triazin-2-yl)amino)-3,3-difluoropiperidin-1-yl)ethan-1-one 729

Fluffy pale-yellow solid (3 mg, 0.006 mmol, 17.1% yield). ¹H NMR (499 MHz, DMSO-d₆) δ ppm 1.52-1.80 (1H, m), 1.81-1.97 (1H, m), 2.02-2.08 (3H, m), 2.60 (3H, s), 2.98 (1H, brt, J=11.36 Hz), 3.57-3.91 (1H, m), 4.09-4.27 (1H, m), 4.42-4.58 (2H, m), 4.63-4.79 (2H, m), 6.20-6.31 (1H, m), 6.47 (1H, tt, J=54.00, 2.75 Hz), 7.12 (1H, d, J=3.01 Hz), 7.39 (1H, dd, J=2.87, 1.51 Hz), 7.60 (1H, br s), 7.83 (1H, d, J=8.76 Hz), 8.00 (1H, d, J=8.49 Hz), 10.67 (1H, br s); ESIMS found for C₂₂H₂₃F₄N₉O m/z 506.2 (M+1).

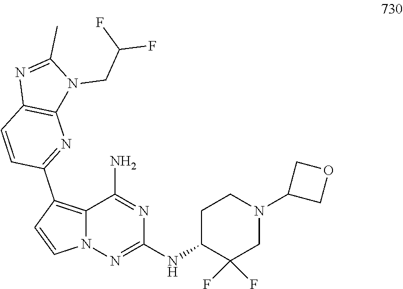 [SMILES: Cc1nc2ccc(-c3ccn4nc(N[C@@H]5CCN(C6COC6)CC5(F)F)nc(N)c34)nc2n1CC(F)F]

### (R)—N²-(3,3-Difluoro-1-(oxetan-3-yl)piperidin-4-yl)-5-(3-(2,2-difluoroethyl)-2-methyl-3H-imidazo[4,5-b]pyridin-5-yl)pyrrolo[2,1-f][1,2,4]triazine-2,4-diamine

Off-white fluffy solid (14 mg, 0.027 mmol, 48.0% yield). ¹H NMR (499 MHz, DMSO-d₆) δ ppm 1.69-1.81 (1H, m), 1.81-1.90 (1H, m), 2.06-2.18 (1H, m), 2.34 (1H, ddd, J=26.60, 12.05, 1.35 Hz), 2.60 (3H, s), 2.74 (1H, br d, J=11.23 Hz), 2.95-3.06 (1H, m), 3.59 (1H, quin, J=6.30 Hz), 4.20-4.36 (1H, m), 4.43 (2H, dt, J=13.89, 6.19 Hz), 4.55 (2H, td, J=6.57, 3.56 Hz), 4.65-4.79 (2H, m), 6.12 (1H, d, J=9.58 Hz), 6.47 (1H, tt, J=54.05, 2.20 Hz), 7.11 (1H, d, J=3.01 Hz), 7.39 (1H, d, J=3.01 Hz), 7.60 (1H, br s), 7.82 (1H, d, J=8.76 Hz), 8.00 (1H, d, J=8.49 Hz), 10.66 (1H, br s); ESIMS found for C₂₃H₂₅F₄N₉O m/z 520.2 (M+1).

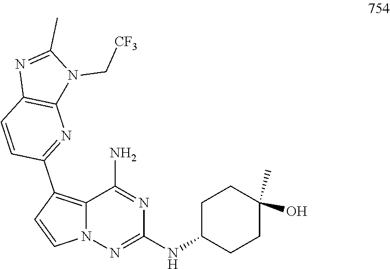 [SMILES: Cc1nc2ccc(-c3ccn4nc(N[C@H]5CC[C@](C)(O)CC5)nc(N)c34)nc2n1CC(F)(F)F]

### (1r,4r)-4-((4-Amino-5-(2-methyl-3-(2,2,2-trifluoroethyl)-3H-imidazo[4,5-b]pyridin-5-yl)pyrrolo[2,1-f][1,2,4]triazin-2-yl)amino)-1-methylcyclohexan-1-ol 754

White solid (10 mg, 0.021 mmol, 30.9% yield). ¹H NMR (499 MHz, DMSO-d₆) δ ppm 1.14 (3H, s), 1.33-1.46 (4H, m), 1.50-1.62 (2H, m), 1.77-1.88 (2H, m), 2.62 (3H, s), 3.62 (1H, dt, J=7.94, 3.97 Hz), 4.21 (1H, br s), 5.15 (2H, q, J=9.22 Hz), 5.74 (1H, d, J=8.21 Hz), 7.08 (1H, d, J=2.74 Hz), 7.39 (1H, d, J=2.74 Hz), 7.54 (1H, br s), 7.84 (1H, d, J=8.76 Hz), 8.02 (1H, d, J=8.49 Hz), 10.31 (1H, br s); ESIMS found for C₂₂H₂₅F₃N₈O m/z 475.2 (M+1).

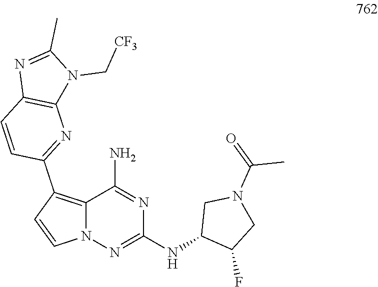 [SMILES: CC(=O)N1C[C@H](F)[C@H](Nc2nc(N)c3c(-c4ccc5nc(C)n(CC(F)(F)F)c5n4)ccn3n2)C1]

### 1-((3R,4S)-3-((4-Amino-5-(2-methyl-3-(2,2,2-trifluoroethyl)-3H-imidazo[4,5-b]pyridin-5-yl)pyrrolo[2,1-f][1,2,4]triazin-2-yl)amino)-4-fluoropyrrolidin-1-yl)ethan-1-one 762

White solid (19 mg, 0.039 mmol, 31.8% yield). ¹H NMR (499 MHz, DMSO-d₆) δ ppm 1.92-2.01 (3H, m), 2.63 (3H, s), 3.49-3.61 (1H, m), 3.62-3.74 (1H, m), 3.75-3.86 (1H, m), 3.91 (1H, t, J=9.03 Hz), 4.27-4.56 (1H, m), 5.16 (2H, q, J=8.94 Hz), 5.20-5.42 (1H, m), 6.33 (1H, dd, J=10.40, 7.67 Hz), 7.14 (1H, dd, J=2.87, 2.05 Hz), 7.43 (1H, dd, J=4.38, 3.01 Hz), 7.70-7.82 (1H, m), 7.87 (1H, d, J=8.49 Hz), 8.04 (1H, d, J=8.49 Hz), 10.46 (1H, br s); ESIMS found for C₂₁H₂₁F₄N₉O m/z 492.2 (M+1).

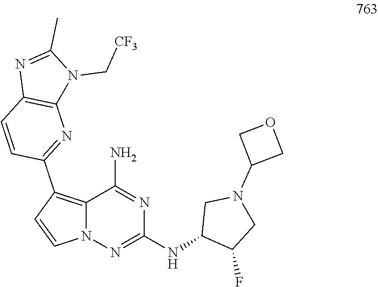 [SMILES: Cc1nc2ccc(-c3ccn4nc(N[C@@H]5CN(C6COC6)C[C@@H]5F)nc(N)c34)nc2n1CC(F)(F)F]

### N²-((3R,4S)-4-Fluoro-1-(oxetan-3-yl)pyrrolidin-3-yl)-5-(2-methyl-3-(2,2,2-trifluoroethyl)-3H-imidazo[4,5-b]pyridin-5-yl)pyrrolo[2,1-f][1,2,4]triazine-2,4-diamine 763

White solid (20 mg, 0.040 mmol, 30.8% yield). ¹H NMR (499 MHz, DMSO-d₆) δ ppm 2.60-2.64 (1H, m), 2.63 (3H, s), 2.76 (1H, ddd, J=30.20, 12.18, 1.23 Hz), 3.00 (1H, t, J=8.21 Hz), 3.15 (1H, ddd, J=33.75, 12.05, 4.38 Hz), 3.74-3.83 (1H, m), 4.23-4.38 (1H, m), 4.46 (2H, t, J=5.89 Hz), 4.59 (2H, t, J=6.57 Hz), 5.19 (1H, dtd, J=55.40, 4.65, 4.65, 1.10 Hz), 5.16 (2H, q, J=9.22 Hz), 6.01 (1H, d, J=7.94 Hz), 7.12 (1H, d, J=3.01 Hz), 7.42 (1H, d, J=2.74 Hz), 7.73 (1H, br s), 7.86 (1H, d, J=8.49 Hz), 8.03 (1H, d, J=8.49 Hz), 10.41 (1H, br s); ESIMS found for C₂₂H₂₃F₄N₉O m/z 506.2 (M+1).

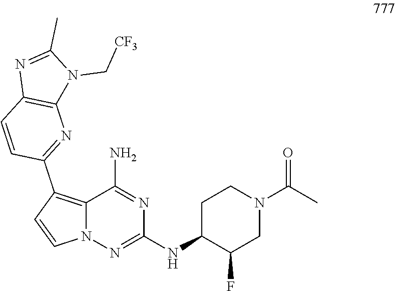 [SMILES: CC(=O)N1CC[C@H](Nc2nc(N)c3c(-c4ccc5nc(C)n(CC(F)(F)F)c5n4)ccn3n2)[C@H](F)C1]

### 1-((3R,4S)-4-((4-Amino-5-(2-methyl-3-(2,2,2-trifluoroethyl)-3H-imidazo[4,5-b]pyridin-5-yl)pyrrolo[2,1-f][1,2,4]triazin-2-yl)amino)-3-fluoropiperidin-1-yl)ethan-1-one 777

Fluffy pale-yellow solid (5 mg, 0.010 mmol, 16.2% yield). ¹H NMR (499 MHz, DMSO-d₆) δ ppm 1.56-1.70 (1H, m), 1.73-1.85 (1H, m), 1.97-2.07 (3H, m), 2.63 (3H, s), 2.67 (1H, td, J=12.87, 3.29 Hz), 2.86 (1H, dd, J=39.80, 14.24 Hz), 3.92-4.06 (1H, m), 4.37-4.49 (1H, m), 4.61-4.76 (1H, m), 4.94 (1H, d, J=49.90 Hz), 5.16 (2H, q, J=9.13 Hz), 5.96 (1H, dd, J=8.08, 5.61 Hz), 7.12 (1H, d, J=3.01 Hz), 7.40 (1H, d, J=2.74 Hz), 7.69 (1H, br s), 7.86 (1H, d, J=8.76 Hz), 8.03 (1H, d, J=8.49 Hz), 10.42 (1H, br s); ESIMS found for C₂₂H₂₃F₄N₉O m/z 253.7 (M/2+1).

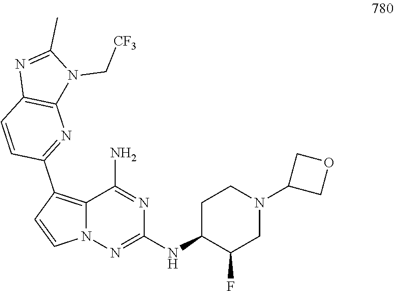 [SMILES: Cc1nc2ccc(-c3ccn4nc(N[C@H]5CCN(C6COC6)C[C@H]5F)nc(N)c34)nc2n1CC(F)(F)F]

### N²-((3R,4S)-3-Fluoro-1-(oxetan-3-yl)piperidin-4-yl)-5-(2-methyl-3-(2,2,2-trifluoroethyl)-3H-imidazo[4,5-b]pyridin-5-yl)pyrrolo[2,1-f][1,2,4]triazine-2,4-diamine 780

Fluffy pale-yellow solid (10 mg, 0.019 mmol, 46.9% yield). ¹H NMR (499 MHz, DMSO-d₆) δ ppm 1.66-1.76 (1H, m), 1.85 (1H, qd, J=12.14, 3.29 Hz), 1.95-2.02 (1H, m), 2.12 (1H, dd, J=37.55, 12.59 Hz), 2.62 (3H, s), 2.74 (1H, br d, J=10.40 Hz), 2.91-3.02 (1H, m), 3.48 (1H, quin, J=6.23 Hz), 3.72-3.89 (1H, m), 4.39 (1H, t, J=6.16 Hz), 4.45 (1H, t, J=6.16 Hz), 4.54 (2H, td, J=6.43, 2.74 Hz), 4.88 (1H, d, J=49.90 Hz), 5.15 (2H, q, J=9.22 Hz), 5.83 (1H, d, J=8.21 Hz), 7.11 (1H, d, J=3.01 Hz), 7.39 (1H, d, J=3.01 Hz), 7.68 (1H, br s), 7.85 (1H, d, J=8.76 Hz), 8.03 (1H, d, J=8.49 Hz), 10.40 (1H, br s); ESIMS found for C₂₃H₂₅F₄N₉O m/z 260.7 (M+1).

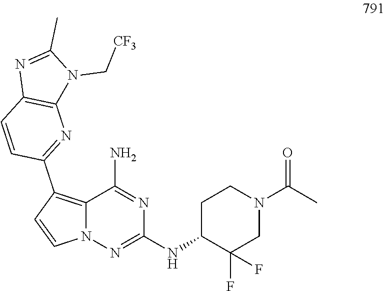 [SMILES: CC(=O)N1CC[C@@H](Nc2nc(N)c3c(-c4ccc5nc(C)n(CC(F)(F)F)c5n4)ccn3n2)C(F)(F)C1]

### (R)-1-(4-((4-Amino-5-(2-methyl-3-(2,2,2-trifluoroethyl)-3H-imidazo[4,5-b]pyridin-5-yl)pyrrolo[2,1-f][1,2,4]triazin-2-yl)amino)-3,3-difluoropiperidin-1-yl)ethan-1-one 791

Fluffy pale-yellow solid (2 mg, 0.004 mmol, 11.0% yield). ¹H NMR (499 MHz, DMSO-d₆) δ ppm 1.52-1.80 (1H, m), 1.81-1.98 (1H, m), 2.02-2.09 (3H, m), 2.63 (3H, s), 2.91-3.03 (1H, m), 3.59-3.93 (1H, m), 4.07-4.30 (1H, m), 4.42-4.59 (2H, m), 5.16 (2H, q, J=8.94 Hz), 6.20-6.30 (1H, m), 7.13 (1H, d, J=3.01 Hz), 7.40 (1H, dd, J=2.74, 1.64 Hz), 7.72 (1H, br s), 7.87 (1H, d, J=8.49 Hz), 8.04 (1H, d, J=8.49 Hz), 10.46 (1H, br s); ESIMS found for C₂₂H₂₂F₅N₉O m/z 524.15 (M+1).

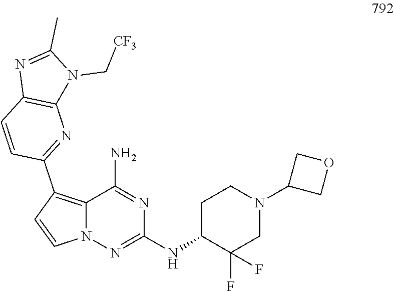 [SMILES: Cc1nc2ccc(-c3ccn4nc(N[C@@H]5CCN(C6COC6)CC5(F)F)nc(N)c34)nc2n1CC(F)(F)F]

### (R)—N²-(3,3-Difluoro-1-(oxetan-3-yl)piperidin-4-yl)-5-(2-methyl-3-(2,2,2-trifluoroethyl)-3H-imidazo[4,5-b]pyridin-5-yl)pyrrolo[2,1-f][1,2,4]triazine-2,4-diamine 792

Fluffy white solid (11 mg, 0.021 mmol, 48.9% yield). ¹H NMR (499 MHz, DMSO-d₆) δ ppm 1.71-1.81 (1H, m), 1.82-1.90 (1H, m), 2.05-2.17 (1H, m), 2.34 (1H, dd, J=26.60, 11.77 Hz), 2.74 (1H, br d, J=10.68 Hz), 2.95-3.06 (1H, m), 3.59 (1H, quin, J=6.16 Hz), 4.21-4.36 (1H, m), 4.43 (2H, dt, J=14.10, 6.09 Hz), 4.55 (2H, td, J=6.57, 3.56 Hz), 5.15 (2H, q, J=9.13 Hz), 6.12 (1H, br d, J=9.58 Hz), 7.12 (1H, d, J=3.01 Hz), 7.40 (1H, d, J=2.74 Hz), 7.72 (1H, br s), 7.86 (1H, d, J=8.76 Hz), 8.03 (1H, d, J=8.49 Hz), 10.45 (1H, br s); ESIMS found for C₂₃H₂₄F₅N₉O m/z 538.2 (M+1).

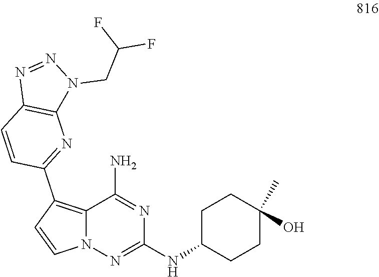 [SMILES: C[C@]1(O)CC[C@H](Nc2nc(N)c3c(-c4ccc5nnn(CC(F)F)c5n4)ccn3n2)CC1]

### (1r,4r)-4-((4-Amino-5-(3-(2,2-difluoroethyl)-3H-[1,2,3]triazolo[4,5-b]pyridin-5-yl)pyrrolo[2,1-f][1,2,4]triazin-2-yl)amino)-1-methylcyclohexan-1-ol 816

Fluffy yellow solid (3.5 mg, 0.008 mmol, 9.7% yield). ¹H NMR (499 MHz, DMSO-d₆) δ ppm 1.14 (3H, s), 1.33-1.47 (4H, m), 1.52-1.63 (2H, m), 1.76-1.88 (2H, m), 3.55-3.68 (1H, m), 4.22 (1H, br s), 5.16 (2H, td, J=16.15, 2.46 Hz), 5.95 (1H, d, J=8.21 Hz), 6.58 (2H, tt, J=54.10, 2.75 Hz), 7.32 (1H, d, J=3.01 Hz), 7.46 (1H, d, J=3.01 Hz), 7.56 (1H, br s), 8.08 (1H, d, J=9.31 Hz), 8.53 (1H, d, J=8.76 Hz), 10.39 (1H, br s); ESIMS found for C₂₀H₂₃F₂N₉O m/z 444.2 (M+1).

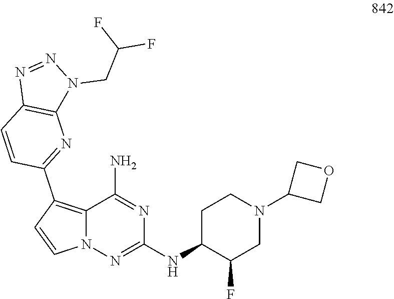 [SMILES: Nc1nc(N[C@H]2CCN(C3COC3)C[C@H]2F)nn2ccc(-c3ccc4nnn(CC(F)F)c4n3)c12]

### 5-(3-(2,2-Difluoroethyl)-3H-[1,2,3]triazolo[4,5-b]pyridin-5-yl)-N²-((3R,4S)-3-fluoro-1-(oxetan-3-yl)piperidin-4-yl)pyrrolo[2,1-f][1,2,4]triazine-2,4-diamine 842

Fluffy yellow solid (7 mg, 0.014 mmol, 48.8% yield). ¹H NMR (499 MHz, DMSO-d₆) δ ppm 1.64-1.77 (1H, m), 1.86 (1H, qd, J=12.09, 3.97 Hz), 1.95-2.03 (1H, m), 2.12 (1H, dd, J=37.50, 12.32 Hz), 2.74 (1H, br d, J=11.23 Hz), 2.90-3.05 (1H, m), 3.48 (1H, quin, J=6.23 Hz), 3.72-3.90 (1H, m), 4.39 (1H, t, J=6.16 Hz), 4.45 (1H, t, J=6.16 Hz), 4.54 (2H, td, J=6.57, 2.74 Hz), 4.88 (1H, d, J=49.90 Hz), 5.16 (2H, td, J=16.22, 2.60 Hz), 6.07 (1H, d, J=8.21 Hz), 6.58 (2H, tt, J=54.00, 2.75 Hz), 7.34 (1H, d, J=3.01 Hz), 7.46 (1H, d, J=3.01 Hz), 7.69 (1H, br s), 8.09 (1H, d, J=9.03 Hz), 8.55 (1H, d, J=9.03 Hz), 10.46 (1H, br s); ESIMS found for C₂₁H₂₃F₃N₁₀O m/z 489.2 (M+1).

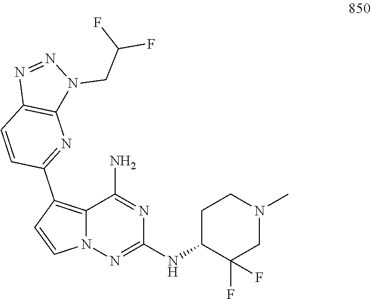 [SMILES: CN1CC[C@@H](Nc2nc(N)c3c(-c4ccc5nnn(CC(F)F)c5n4)ccn3n2)C(F)(F)C1]

### (R)—N²-(3,3-Difluoro-1-methylpiperidin-4-yl)-5-(3-(2,2-difluoroethyl)-3H-[1,2,3]triazolo[4,5-b]pyridin-5-yl)pyrrolo[2,1-f][1,2,4]triazine-2,4-diamine 850

Fluffy yellow solid (20 mg, 0.043 mmol, 52.5% yield). ¹H NMR (499 MHz, DMSO-d₆) δ ppm 1.70-1.87 (2H, m), 2.07-2.17 (1H, m), 2.25 (3H, s), 2.33 (1H, dd, J=27.40, 11.66 Hz), 2.78 (1H, brd, J=12.05 Hz), 2.98-3.10 (1H, m), 4.15-4.32 (1H, m), 5.16 (2H, td, J=16.15, 2.46 Hz), 6.25 (1H, d, J=9.58 Hz), 6.58 (1H, tt, J=54.05, 2.75 Hz), 7.36 (1H, d, J=3.01 Hz), 7.47 (1H, d, J=3.01 Hz), 7.74 (1H, br s), 8.10 (1H, d, J=9.31 Hz), 8.55 (1H, d, J=9.03 Hz), 10.49 (1H, br s); ESIMS found for C₁₉H₂₀F₄N₁₀ m/z 465.2 (M+1).

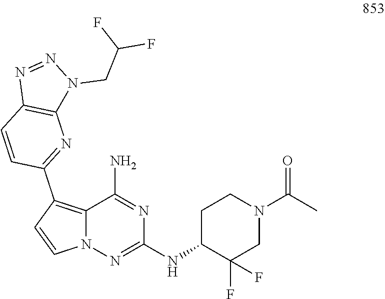 [SMILES: CC(=O)N1CC[C@@H](Nc2nc(N)c3c(-c4ccc5nnn(CC(F)F)c5n4)ccn3n2)C(F)(F)C1]

### (R)-1-(4-((4-Amino-5-(3-(2,2-difluoroethyl)-3H-[1,2,3]triazolo[4,5-b]pyridin-5-yl)pyrrolo[2,1-f][1,2,4]triazin-2-yl)amino)-3,3-difluoropiperidin-1-yl)ethan-1-one 853

Fluffy yellow solid (1.3 mg, 0.003 mmol, 6.1% yield). ¹H NMR (499 MHz, DMSO-d₆) δ ppm 1.53-1.80 (1H, m), 1.82-1.99 (1H, m), 2.02-2.08 (3H, m), 2.88-3.02 (1H, m), 3.57-3.93 (1H, m), 4.05-4.31 (1H, m), 4.41-4.59 (2H, m), 5.17 (2H, td, J=16.22, 2.33 Hz), 6.59 (1H, tt, J=54.00, 2.75 Hz), 6.68 (1H, s), 7.37 (1H, d, J=3.01 Hz), 7.47 (1H, dd, J=2.74, 1.37 Hz), 7.73 (1H, br s), 8.11 (1H, d, J=9.03 Hz), 8.56 (1H, d, J=9.03 Hz), 10.52 (1H, br s); ESIMS found for C₂₀H₂₀F₄N₁₀O m/z 493.15 (M+1).

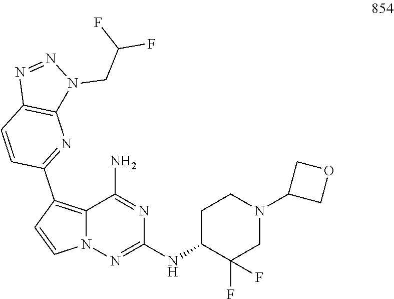 [SMILES: Nc1nc(N[C@@H]2CCN(C3COC3)CC2(F)F)nn2ccc(-c3ccc4nnn(CC(F)F)c4n3)c12]

### (R)—N²-(3,3-Difluoro-1-(oxetan-3-yl)piperidin-4-yl)-5-(3-(2,2-difluoroethyl)-3H-[1,2,3]triazolo[4,5-b]pyridin-5-yl)pyrrolo[2,1-f][1,2,4]triazine-2,4-diamine 854

Fluffy yellow solid (9 mg, 0.018 mmol, 42.5% yield). ¹H NMR (499 MHz, DMSO-d₆) δ ppm 1.71-1.81 (1H, m), 1.86 (1H, br dd, J=8.62, 4.52 Hz), 2.06-2.17 (1H, m), 2.35 (1H, dd, J=26.35, 11.77 Hz), 2.74 (1H, br d, J=11.23 Hz), 2.95-3.06 (1H, m), 3.59 (1H, quin, J=6.37 Hz), 4.20-4.36 (1H, m), 4.43 (2H, dt, J=14.24, 6.30 Hz), 4.55 (2H, td, J=6.64, 3.70 Hz), 5.17 (2H, td, J=16.15, 2.19 Hz), 6.34 (1H, br d, J=9.31 Hz), 6.59 (1H, tt, J=54.05, 2.75 Hz), 7.36 (1H, d, J=3.01 Hz), 7.47 (1H, d, J=3.01 Hz), 7.73 (1H, br s), 8.10 (1H, d, J=9.03 Hz), 8.55 (1H, d, J=9.03 Hz), 10.50 (1H, br s) ESIMS found for C₂₁H₂₂F₄N₁₀O m/z 507.2 (M+1).

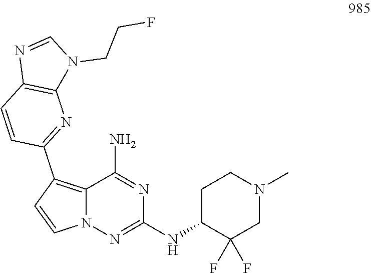 [SMILES: CN1CC[C@@H](Nc2nc(N)c3c(-c4ccc5ncn(CCF)c5n4)ccn3n2)C(F)(F)C1]

### (R)—N²-(3,3-Difluoro-1-methylpiperidin-4-yl)-5-(3-(2-fluoroethyl)-3H-imidazo[4,5-b]pyridin-5-yl)pyrrolo[2,1-f][1,2,4]triazine-2,4-diamine 985

White solid (15 mg, 0.034 mmol, 50.8% yield). ¹H NMR (499 MHz, DMSO-d₆) δ ppm 1.70-1.79 (1H, m), 1.80-1.85 (1H, m), 2.13 (1H, br t, J=10.40 Hz), 2.25 (3H, s), 2.32 (1H, ddd, J=27.10, 12.30, 1.65 Hz), 2.77 (1H, br d, J=11.50 Hz), 2.98-3.10 (1H, m), 4.13-4.31 (1H, m), 4.58 (2H, dt, J=27.70, 4.65 Hz), 4.82 (2H, dt, J=47.15, 4.65 Hz), 6.07 (1H, d, J=9.31 Hz), 7.16 (1H, d, J=3.01 Hz), 7.40 (1H, d, J=3.01 Hz), 7.65 (1H, br s), 7.88 (1H, d, J=8.49 Hz), 8.13 (1H, d, J=8.76 Hz), 8.42 (1H, s), 10.81 (1H, br s); ESIMS found for C₂₀H₂₂F₃N₉ m/z 446.1 (M+1).

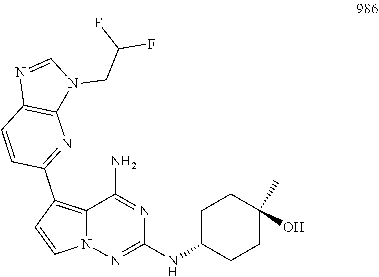 [SMILES: C[C@]1(O)CC[C@H](Nc2nc(N)c3c(-c4ccc5ncn(CC(F)F)c5n4)ccn3n2)CC1]

### (1r,4r)-4-((4-Amino-5-(3-(2,2-difluoroethyl)-3H-imidazo[4,5-b]pyridin-5-yl)pyrrolo[2,1-f][1,2,4]triazin-2-yl)amino)-1-methylcyclohexan-1-ol 986

White solid (8 mg, 0.018 mmol, 26.5% yield). ¹H NMR (499 MHz, DMSO-d₆) δ ppm 1.14 (3H, s), 1.34-1.46 (4H, m), 1.51-1.62 (2H, m), 1.77-1.89 (2H, m), 3.62 (1H, dt, J=8.08, 3.90 Hz), 4.20 (1H, br s), 4.74 (2H, td, J=16.50, 2.33 Hz), 5.77 (1H, d, J=8.21 Hz), 6.47 (1H, tt, J=54.45, 2.75 Hz), 7.11 (1H, d, J=3.01 Hz), 7.40 (1H, d, J=3.01 Hz), 7.45 (1H, br s), 7.87 (1H, d, J=8.76 Hz), 8.14 (1H, d, J=8.49 Hz), 8.39 (1H, s), 10.49 (1H, br s); ESIMS found for C₂₁H₂₄F₂N₈O m/z 443.2 (M+1).

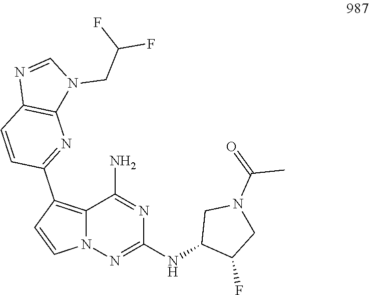 [SMILES: CC(=O)N1C[C@H](F)[C@H](Nc2nc(N)c3c(-c4ccc5ncn(CC(F)F)c5n4)ccn3n2)C1]

### 1-((3R,4S)-3-((4-Amino-5-(3-(2,2-difluoroethyl)-3H-imidazo[4,5-b]pyridin-5-yl)pyrrolo[2,1-f][1,2,4]triazin-2-yl)amino)-4-fluoropyrrolidin-1-yl)ethan-1-one 987

White solid (15 mg, 0.033 mmol, 26.9% yield). ¹H NMR (499 MHz, DMSO-d₆) δ ppm 1.98 (3H, s), 3.41-3.60 (1H, m), 3.62-3.73 (1H, m), 3.75-3.87 (1H, m), 3.91 (1H, t, J=9.03 Hz), 4.25-4.55 (1H, m), 4.75 (2H, td, J=16.45, 2.20 Hz), 5.18-5.43 (1H, m), 6.36 (1H, dd, J=11.50, 7.67 Hz), 6.48 (1H, tt, J=54.30, 2.45 Hz), 7.17 (1H, dd, J=3.01, 1.64 Hz), 7.43 (1H, dd, J=4.24, 2.87 Hz), 7.62-7.70 (1H, m), 7.90 (1H, d, J=8.76 Hz), 8.16 (1H, d, J=8.76 Hz), 8.41 (1H, s), 10.63 (1H, br s); ESIMS found for C₂₀H₂₀F₃N₉O m/z 460.2 M+1).

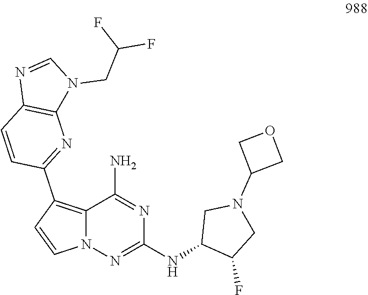 [SMILES: Nc1nc(N[C@@H]2CN(C3COC3)C[C@@H]2F)nn2ccc(-c3ccc4ncn(CC(F)F)c4n3)c12]

### 5-(3-(2,2-Difluoroethyl)-3H-imidazo[4,5-b]pyridin-5-yl)-N²-((3R,4S)-4-fluoro-1-(oxetan-3-yl)pyrrolidin-3-yl)pyrrolo[2,1-f][1,2,4]triazine-2,4-diamine 988

White solid (21 mg, 0.044 mmol, 34.5% yield). ¹H NMR (499 MHz, DMSO-d₆) δ ppm 2.62 (1H, t, J=9.03 Hz), 2.76 (1H, ddd, J=30.15, 12.05, 1.10 Hz), 2.99 (1H, t, J=8.21 Hz), 3.16 (1H, ddd, J=33.75, 12.18, 4.52 Hz), 3.78 (1H, quin, J=6.16 Hz), 4.22-4.37 (1H, m), 4.46 (2H, t, J=6.02 Hz), 4.59 (2H, t, J=6.57 Hz), 4.75 (2H, td, J=16.43, 2.19 Hz), 5.19 (1H, dtd, J=55.95, 4.40, 4.40, 1.40 Hz), 6.05 (1H, d, J=8.21 Hz), 6.48 (1H, tt, J=54.55, 2.60 Hz), 7.16 (1H, d, J=2.74 Hz), 7.42 (1H, d, J=2.74 Hz), 7.64 (1H, br s), 7.89 (1H, d, J=8.76 Hz), 8.15 (1H, d, J=8.49 Hz), 8.41 (1H, s), 10.58 (1H, br s); ESIMS found for C₂₁H₂₂F₃N₉O m/z 474.2 (M+1).

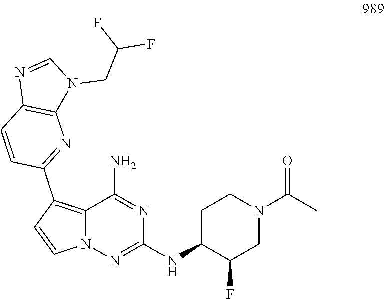 [SMILES: CC(=O)N1CC[C@H](Nc2nc(N)c3c(-c4ccc5ncn(CC(F)F)c5n4)ccn3n2)[C@H](F)C1]

### 1-((3R,4S)-4-((4-Amino-5-(3-(2,2-difluoroethyl)-3H-imidazo[4,5-b]pyridin-5-yl)pyrrolo[2,1-f][1,2,4]triazin-2-yl)amino)-3-fluoropiperidin-1-yl)ethan-1-one 989

Fluffy pale-yellow solid (10 mg, 0.021 mmol, 34.5% yield). ¹H NMR (499 MHz, DMSO-d₆) δ ppm 1.58-1.72 (1H, m), 1.73-1.85 (1H, m), 1.97-2.05 (3H, m), 2.67 (1H, td, J=12.66, 3.70 Hz), 2.86 (1H, dd, J=39.75, 13.96 Hz), 3.92-4.05 (1H, m), 4.07-4.16 (1H, m), 4.43 (1H, br dd, J=11.22, 2.19 Hz), 4.70-4.82 (2H, m), 4.94 (1H, d, J=49.90 Hz), 6.00 (1H, dd, J=7.94, 5.20 Hz), 6.48 (1H, tt, J=54.00, 2.75 Hz), 7.15 (1H, d, J=3.01 Hz), 7.40 (1H, d, J=2.74 Hz), 7.59 (1H, br s), 7.89 (1H, d, J=8.76 Hz), 8.15 (1H, d, J=8.49 Hz), 8.40 (1H, s), 10.59 (1H, br s); ESIMS found for C₂₁H₂₂F₃N₉O m/z 474.2 (M+1).

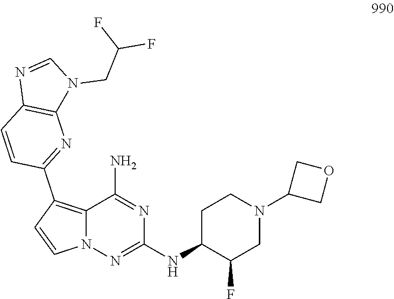 [SMILES: Nc1nc(N[C@H]2CCN(C3COC3)C[C@H]2F)nn2ccc(-c3ccc4ncn(CC(F)F)c4n3)c12]

### 5-(3-(2,2-Difluoroethyl)-3H-imidazo[4,5-b]pyridin-5-yl)-N²-((3R,4S)-3-fluoro-1-(oxetan-3-yl)piperidin-4-yl)pyrrolo[2,1-f][1,2,4]triazine-2,4-diamine 990

Fluffy pale-yellow solid (14 mg, 0.029 mmol, 69.9% yield). ¹H NMR (499 MHz, DMSO-d₆) δ ppm 1.64-1.75 (1H, m), 1.85 (1H, qd, J=12.09, 3.70 Hz), 1.95-2.02 (1H, m), 2.12 (1H, dd, J=37.30, 12.59 Hz), 2.74 (1H, br d, J=10.68 Hz), 2.90-3.03 (1H, m), 3.48 (1H, quin, J=6.30 Hz), 3.73-3.87 (1H, m), 4.39 (1H, t, J=6.16 Hz), 4.45 (1H, t, J=6.16 Hz), 4.53 (2H, td, J=6.57, 2.74 Hz), 4.74 (2H, td, J=16.48, 2.20 Hz), 4.88 (1H, d, J=49.90 Hz), 5.87 (1H, d, J=8.21 Hz), 6.48 (1H, tt, J=54.00, 2.75 Hz), 7.14 (1H, d, J=3.01 Hz), 7.40 (1H, d, J=2.74 Hz), 7.58 (1H, br s), 7.88 (1H, d, J=8.76 Hz), 8.15 (1H, d, J=8.76 Hz), 8.40 (1H, s), 10.58 (1H, br s); ESIMS found for C₂₂H₂₄F₃N₉O m/z 488.2 (M+1).

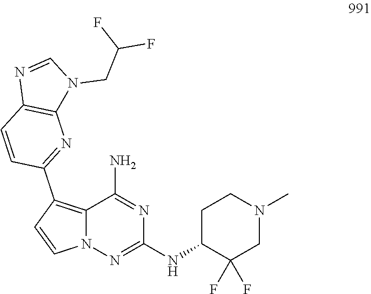 [SMILES: CN1CC[C@@H](Nc2nc(N)c3c(-c4ccc5ncn(CC(F)F)c5n4)ccn3n2)C(F)(F)C1]

### (R)—N²-(3,3-Difluoro-1-methylpiperidin-4-yl)-5-(3-(2,2-difluoroethyl)-3H-imidazo[4,5-b]pyridin-5-yl)pyrrolo[2,1-f][1,2,4]triazine-2,4-diamine 991

White solid (20 mg, 0.043 mmol, 65.1% yield). ¹H NMR (499 MHz, DMSO-d₆) δ ppm 1.70-1.79 (1H, m), 1.79-1.86 (1H, m), 2.08-2.19 (1H, m), 2.25 (3H, s), 2.32 (1H, ddd, J=26.85, 12.05, 1.37 Hz), 2.77 (1H, br d, J=11.50 Hz), 3.00-3.10 (1H, m), 4.15-4.29 (1H, m), 4.75 (2H, td, J=16.36, 2.05 Hz), 6.07 (1H, d, J=9.58 Hz), 6.48 (1H, tt, J=54.30, 2.50 Hz), 7.15 (1H, d, J=3.01 Hz), 7.41 (1H, d, J=2.74 Hz), 7.63 (1H, br s), 7.89 (1H, d, J=8.76 Hz), 8.15 (1H, d, J=8.76 Hz), 8.40 (1H, s), 10.61 (1H, br s); ESIMS found for C₂₀H₂₁F₄N₉ m/z 464.1 (M+1).

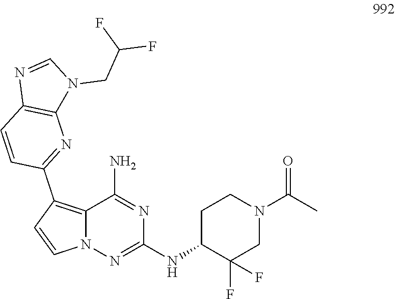 [SMILES: CC(=O)N1CC[C@@H](Nc2nc(N)c3c(-c4ccc5ncn(CC(F)F)c5n4)ccn3n2)C(F)(F)C1]

### (R)-1-(4-((4-Amino-5-(3-(2,2-difluoroethyl)-3H-imidazo[4,5-b]pyridin-5-yl)pyrrolo[2,1-f][1,2,4]triazin-2-yl)amino)-3,3-difluoropiperidin-1-yl)ethan-1-one 992

Fluffy pale-yellow solid (8 mg, 0.016 mmol, 46.8% yield). ¹H NMR (499 MHz, DMSO-d₆) δ ppm 1.53-1.78 (1H, m), 1.80-1.98 (1H, m), 2.02-2.08 (3H, m), 2.91-3.04 (1H, m), 3.65 (1H, dd, J=28.00, 14.24 Hz), 4.07-4.27 (1H, m), 4.41-4.59 (2H, m), 4.75 (2H, td, J=16.50, 2.33 Hz), 6.29 (1H, dd, J=9.31, 6.02 Hz), 6.48 (1H, tt, J=54.00, 2.75 Hz), 7.16 (1H, d, J=3.01 Hz), 7.41 (1H, dd, J=2.87, 1.51 Hz), 7.62 (1H, br s), 7.89 (1H, d, J=8.76 Hz), 8.16 (1H, d, J=8.49 Hz), 8.41 (1H, s), 10.64 (1H, br s); ESIMS found for C₂₁H₂₁F₄N₉O m/z 492.2 (M+1).

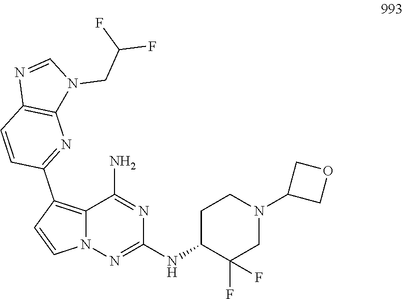 [SMILES: Nc1nc(N[C@@H]2CCN(C3COC3)CC2(F)F)nn2ccc(-c3ccc4ncn(CC(F)F)c4n3)c12]

### (R)—N²-(3,3-Difluoro-1-(oxetan-3-yl)piperidin-4-yl)-5-(3-(2,2-difluoroethyl)-3H-imidazo[4,5-b]pyridin-5-yl)pyrrolo[2,1-f][1,2,4]triazine-2,4-diamine 993

Fluffy white solid (13 mg, 0.026 mmol, 61.5% yield). ¹H NMR (499 MHz, DMSO-d₆) δ ppm 1.71-1.81 (1H, m), 1.82-1.89 (1H, m), 2.06-2.17 (1H, m), 2.35 (1H, dd, J=26.60, 11.23 Hz), 2.74 (1H, br d, J=11.77 Hz), 2.95-3.05 (1H, m), 3.59 (1H, quin, J=6.37 Hz), 4.19-4.35 (1H, m), 4.43 (2H, dt, J=14.10, 6.09 Hz), 4.55 (2H, td, J=6.64, 3.70 Hz), 4.75 (2H, td, J=16.45, 2.20 Hz), 6.15 (1H, br d, J=9.58 Hz), 6.48 (1H, tt, J=54.00, 2.75 Hz), 7.15 (1H, d, J=3.01 Hz), 7.40 (1H, d, J=2.74 Hz), 7.62 (1H, br s), 7.89 (1H, d, J=8.76 Hz), 8.15 (1H, d, J=8.76 Hz), 8.40 (1H, s), 10.62 (1H, br s); ESIMS found for C₂₂H₂₃F₄N₉O m/z 506.25 (M+1).

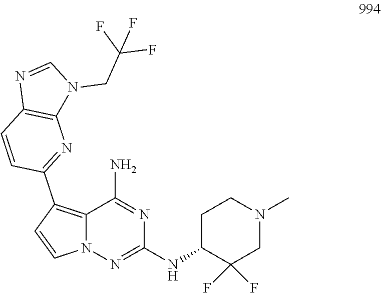 [SMILES: CN1CC[C@@H](Nc2nc(N)c3c(-c4ccc5ncn(CC(F)(F)F)c5n4)ccn3n2)C(F)(F)C1]

### (R)—N²-(3,3-Difluoro-1-methylpiperidin-4-yl)-5-(3-(2,2,2-trifluoroethyl)-3H-imidazo[4,5-b]pyridin-5-yl)pyrrolo[2,1-f][1,2,4]triazine-2,4-diamine 994

White solid (18 mg, 0.037 mmol, 56.4% yield). ¹H NMR (499 MHz, DMSO-d₆) δ ppm 1.71-1.79 (1H, m), 1.80-1.86 (1H, m), 2.08-2.19 (1H, m), 2.25 (3H, s), 2.32 (1H, ddd, J=26.35, 12.59, 1.64 Hz), 2.77 (1H, br d, J=11.50 Hz), 2.98-3.09 (1H, m), 4.14-4.31 (1H, m), 5.20 (2H, q, J=9.22 Hz), 6.07 (1H, d, J=9.58 Hz), 7.17 (1H, d, J=3.01 Hz), 7.42 (1H, d, J=2.74 Hz), 7.72 (1H, br s), 7.93 (1H, d, J=8.76 Hz), 8.18 (1H, d, J=8.49 Hz), 8.48 (1H, s), 10.48 (1H, br s); ESIMS found for C₂₀H₂₀F₅N₉ m/z 482.2 (M+1).

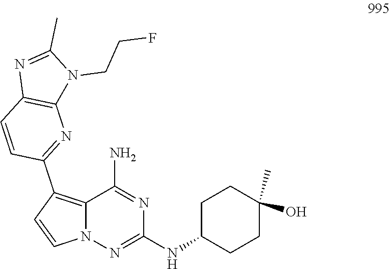 [SMILES: Cc1nc2ccc(-c3ccn4nc(N[C@H]5CC[C@](C)(O)CC5)nc(N)c34)nc2n1CCF]

### (1r,4r)-4-((4-Amino-5-(3-(2-fluoroethyl)-2-methyl-3H-imidazo[4,5-b]pyridin-5-yl)pyrrolo[2,1-f][1,2,4]triazin-2-yl)amino)-1-methylcyclohexan-1-ol 995

White solid (2 mg, 0.005 mmol, 6.7% yield). ¹H NMR (499 MHz, DMSO-d₆) δ ppm 1.14 (3H, s), 1.33-1.45 (4H, m), 1.50-1.61 (2H, m), 1.74-1.86 (2H, m), 2.59 (3H, s), 3.54-3.68 (1H, m), 4.21 (1H, br s), 4.54 (2H, dt, J=27.45, 4.52 Hz), 4.78 (2H, dt, J=46.90, 4.65 Hz), 5.74 (1H, d, J=8.21 Hz), 7.08 (1H, d, J=2.74 Hz), 7.38 (1H, d, J=2.74 Hz), 7.46 (1H, br s), 7.79 (1H, d, J=8.49 Hz), 7.97 (1H, d, J=8.49 Hz), 10.71 (1H, br s); ESIMS found for C₂₂H₂₇FN₈O m/z 439.25 (M+1).

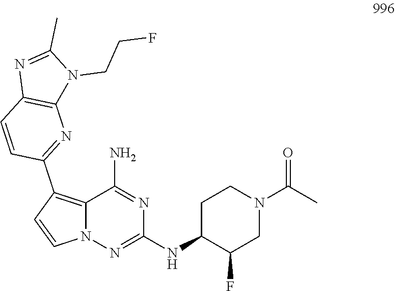 [SMILES: CC(=O)N1CC[C@H](Nc2nc(N)c3c(-c4ccc5nc(C)n(CCF)c5n4)ccn3n2)[C@H](F)C1]

### 1-((3R,4S)-4-((4-Amino-5-(3-(2-fluoroethyl)-2-methyl-3H-imidazo[4,5-b]pyridin-5-yl)pyrrolo[2,1-f][1,2,4]triazin-2-yl)amino)-3-fluoropiperidin-1-yl)ethan-1-one 996

Fluffy pale-yellow solid (3 mg, 0.006 mmol, 10.4% yield). ¹H NMR (499 MHz, DMSO-d₆) δ ppm 1.58-1.71 (1H, m), 1.73-1.85 (1H, m), 1.96-2.07 (3H, m), 2.59 (3H, s), 2.67 (1H, td, J=12.73, 3.29 Hz), 2.86 (1H, dd, J=40.05, 13.96 Hz), 3.91-4.04 (1H, m), 4.38-4.47 (1H, m), 4.55 (2H, dt, J=27.40, 4.65 Hz), 4.64-4.72 (1H, m), 4.78 (2H, dt, J=47.20, 4.65 Hz), 4.94 (1H, d, J=49.90 Hz), 5.96 (1H, dd, J=8.08, 5.61 Hz), 7.12 (1H, d, J=3.01 Hz), 7.38 (1H, d, J=3.01 Hz), 7.60 (1H, br s), 7.81 (1H, d, J=8.49 Hz), 7.98 (1H, d, J=8.49 Hz), 10.81 (1H, br s); ESIMS found for C₂₂H₂₅F₂N₉O m/z 235.65 (M/2+1).

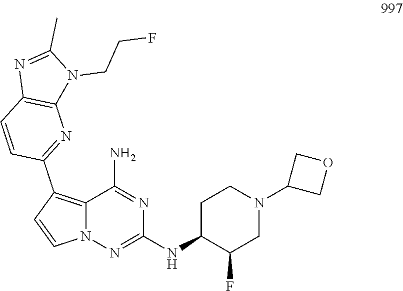 [SMILES: Cc1nc2ccc(-c3ccn4nc(N[C@H]5CCN(C6COC6)C[C@H]5F)nc(N)c34)nc2n1CCF]

### N²-((3R,4S)-3-Fluoro-1-(oxetan-3-yl)piperidin-4-yl)-5-(3-(2-fluoroethyl)-2-methyl-3H-imidazo[4,5-b]pyridn-5-yl)pyrrolo[2,1-f][1,2,4]triazine-2,4-diamine 997

Fluffy pale-yellow solid (4 mg, 0.008 mmol, 20.1% yield). ¹H NMR (499 MHz, DMSO-d₆) δ ppm 1.65-1.76 (1H, m), 1.85 (1H, qd, J=12.23, 3.83 Hz), 1.94-2.02 (1H, m), 2.12 (1H, dd, J=37.60, 12.59 Hz), 2.59 (3H, s), 2.74 (1H, br d, J=11.23 Hz), 2.91-3.04 (1H, m), 3.48 (1H, dt, J=12.80, 6.33 Hz), 3.69-3.90 (1H, m), 4.39 (1H, t, J=6.16 Hz), 4.45 (1H, t, J=6.16 Hz), 4.50-4.60 (4H, m), 4.78 (2H, dt, J=46.90, 4.73 Hz), 4.88 (1H, d, J=49.90 Hz), 5.83 (1H, d, J=8.21 Hz), 7.11 (1H, d, J=3.01 Hz), 7.38 (1H, d, J=2.74 Hz), 7.59 (1H, br s), 7.80 (1H, d, J=8.76 Hz), 7.98 (1H, d, J=8.49 Hz), 10.80 (1H, br s); ESIMS found for C₂₃H₂₇F₂N₉O m/z 242.7 (M/2+1).

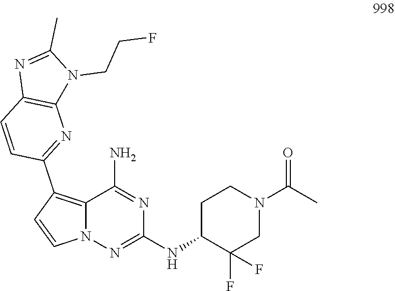 [SMILES: CC(=O)N1CC[C@@H](Nc2nc(N)c3c(-c4ccc5nc(C)n(CCF)c5n4)ccn3n2)C(F)(F)C1]

### (R)-1-(4-((4-Amino-5-(3-(2-fluoroethyl)-2-methyl-3H-imidazo[4,5-b]pyridin-5-yl)pyrrolo[2,1-f][1,2,4]triazin-2-yl)amino)-3,3-difluoropiperidin-1-yl)ethan-1-one 998

Fluffy yellow solid (0.31 mg, 0.0006 mmol, 1.8% yield). ¹H NMR (499 MHz, DMSO-d₆) δ ppm 1.52-1.78 (1H, m), 1.81-1.96 (1H, m), 2.02-2.08 (3H, m), 2.59 (3H, s), 2.90-3.03 (1H, m), 3.84 (1H, br d, J=13.69 Hz), 4.07-4.26 (1H, m), 4.42-4.52 (2H, m), 4.55 (2H, dt, J=27.75, 4.65 Hz), 4.78 (2H, dt, J=46.90, 4.65 Hz), 6.26 (1H, dd, J=9.58, 6.57 Hz), 7.13 (1H, d, J=3.01 Hz), 7.39 (1H, dd, J=2.74, 1.64 Hz), 7.63 (1H, br s), 7.81 (1H, d, J=8.76 Hz), 7.99 (1H, d, J=8.49 Hz), 8.50 (3H, s), 10.85 (1H, br s); ESIMS found for C₂₂H₂₄F₃N₉O m/z 488.2 (M+1).

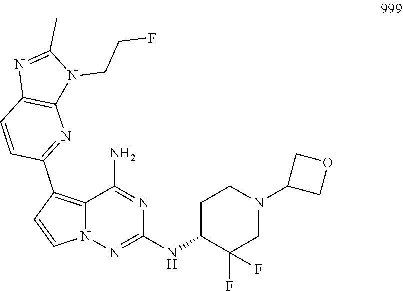 [SMILES: Cc1nc2ccc(-c3ccn4nc(N[C@@H]5CCN(C6COC6)CC5(F)F)nc(N)c34)nc2n1CCF]

### (R)—N²-(3,3-Difluoro-1-(oxetan-3-yl)piperidin-4-yl)-5-(3-(2-fluoroethyl)-2-methyl-3H-imidazo[4,5-b]pyridin-5-yl)pyrrolo[2,1-f][1,2,4]triazine-2,4-diamine 999

Fluffy white solid (6 mg, 0.012 mmol, 28.6% yield). ¹H NMR (499 MHz, DMSO-d₆) δ ppm 1.70-1.80 (1H, m), 1.82-1.89 (1H, m), 2.06-2.17 (1H, m), 2.34 (1H, dd, J=26.35, 12.05 Hz), 2.59 (3H, s), 2.69-2.79 (1H, m), 2.93-3.05 (1H, m), 3.59 (1H, quin, J=6.30 Hz), 4.18-4.35 (1H, m), 4.43 (2H, dt, J=13.83, 6.23 Hz), 4.50-4.56 (2H, m), 4.55 (2H, dt, J=27.45, 4.65 Hz), 4.78 (2H, dt, J=47.20, 4.73 Hz), 6.12 (1H, br d, J=9.58 Hz), 7.12 (1H, d, J=3.01 Hz), 7.38 (1H, d, J=2.74 Hz), 7.63 (1H, br s), 7.81 (1H, d, J=8.49 Hz), 7.98 (1H, d, J=8.49 Hz), 10.85 (1H, br s); ESIMS found for C₂₃H₂₆F₃N₉O m/z 251.7 (M/2+1).

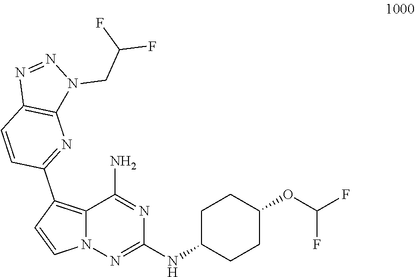 [SMILES: Nc1nc(N[C@H]2CC[C@@H](OC(F)F)CC2)nn2ccc(-c3ccc4nnn(CC(F)F)c4n3)c12]

### 5-(3-(2,2-Difluoroethyl)-3H-[1,2,3]triazolo[4,5-b]pyridin-5-yl)-N²-(cis-4-(difluoromethoxy)cyclohexyl)pyrrolo[2,1-f][1,2,4]triazine-2,4-diamine 1000

Fluffy yellow solid (15 mg, 0.031 mmol, 49.4% yield). ¹H NMR (499 MHz, DMSO-d₆) δ ppm 1.57-1.68 (4H, m), 1.70-1.78 (2H, m), 1.82-1.90 (2H, m), 3.60-3.72 (1H, m), 4.27 (1H, br s), 5.16 (2H, td, J=16.22, 2.33 Hz), 6.13 (1H, d, J=7.94 Hz), 6.58 (1H, tt, J=54.00, 2.75 Hz), 6.72 (1H, t, J=77.05 Hz), 7.32 (1H, d, J=3.01 Hz), 7.45 (1H, d, J=2.74 Hz), 7.57 (1H, br s), 8.08 (1H, d, J=9.03 Hz), 8.53 (1H, d, J=9.03 Hz), 10.40 (1H, br s); ESIMS found for C₂₀H₂₁F₄N₉O m/z 480.2 (M+1).

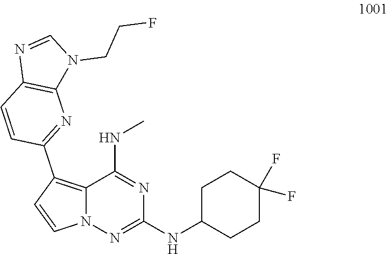 [SMILES: CNc1nc(NC2CCC(F)(F)CC2)nn2ccc(-c3ccc4ncn(CCF)c4n3)c12]

### N²-(4,4-Difluorocyclohexyl)-5-(3-(2-fluoroethyl)-3H-imidazo[4,5-b]pyridin-5-yl)-N⁴-methylpyrrolo[2,1-f][1,2,4]triazine-2,4-diamine 1001

Off-white solid (10 mg, 0.023 mmol, 54.6% yield). ¹H NMR (499 MHz, DMSO-d₆) δ ppm 1.55-1.68 (2H, m), 1.82-2.00 (4H, m), 2.01-2.14 (2H, m), 3.10 (3H, d, J=4.65 Hz), 3.78 (1H, q, J=8.30 Hz), 4.65 (2H, dt, J=27.45, 4.79 Hz), 4.93 (2H, dt, J=46.90, 4.79 Hz), 6.16 (1H, d, J=7.94 Hz), 7.12 (1H, d, J=3.01 Hz), 7.37 (1H, d, J=3.01 Hz), 7.88 (1H, d, J=8.76 Hz), 8.13 (1H, d, J=8.49 Hz), 8.45 (1H, s), 11.11 (1H, q, J=4.65 Hz); ESIMS found for C₂₁H₂₃F₃N₈ m/z 223.2 (M/2+1).

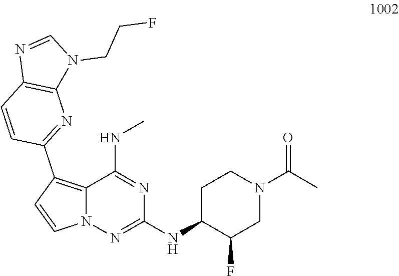 [SMILES: CNc1nc(N[C@H]2CCN(C(C)=O)C[C@H]2F)nn2ccc(-c3ccc4ncn(CCF)c4n3)c12]

### 1-((3R,4S)-3-Fluoro-4-((5-(3-(2-fluoroethyl)-3H-imidazo[4,5-b]pyridin-5-yl)-4-(methylamino)pyrrolo[2,1-f][1,2,4]triazin-2-yl)amino)piperidin-1-yl)ethan-1-one 1002

Fluffy white solid (11 mg, 0.023 mmol, 61.4% yield). ¹H NMR (499 MHz, DMSO-d₆) δ ppm 1.61-1.71 (1H, m), 1.73-1.86 (1H, m), 1.98-2.05 (3H, m), 2.60-2.93 (1H, m), 3.11 (3H, d, J=4.65 Hz), 3.83-3.92 (1H, m), 3.93-4.05 (1H, m), 4.07-4.18 (1H, m), 4.40-4.49 (1H, m), 4.66 (2H, dt, J=27.45, 4.93 Hz), 4.93 (2H, dt, J=46.90, 4.79 Hz), 4.97 (1H, d, J=49.10 Hz), 6.09 (1H, dd, J=7.94, 4.11 Hz), 7.14 (1H, d, J=3.01 Hz), 7.37 (1H, d, J=2.74 Hz), 7.89 (1H, d, J=8.76 Hz), 8.14 (1H, d, J=8.49 Hz), 8.45 (1H, s), 11.17 (1H, q, J=4.29 Hz); ESIMS found for C₂₂H₂₅F₂N₉O m/z 235.65 (M/2+1).

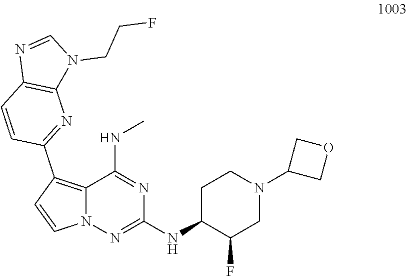 [SMILES: CNc1nc(N[C@H]2CCN(C3COC3)C[C@H]2F)nn2ccc(-c3ccc4ncn(CCF)c4n3)c12]

### N²-((3R,4S)-3-Fluoro-1-(oxetan-3-yl)piperidin-4-yl)-5-(3-(2-fluoroethyl)-3H-imidazo[4,5-b]pyridin-5-yl)-N4-methylpyrrolo[2,1-f][1,2,4]triazine-2,4-diamine 1003

Off-white fluffy solid (10 mg, 0.021 mmol, 56.4% yield). ¹H NMR (499 MHz, DMSO-d₆) δ ppm 1.65-1.76 (1H, m), 1.87 (1H, qd, J=12.09, 3.70 Hz), 1.96-2.03 (1H, m), 2.13 (1H, dd, J=37.00, 12.59 Hz), 2.74 (1H, br d, J=12.05 Hz), 2.97 (1H, brt, J=10.54 Hz), 3.11 (3H, d, J=4.93 Hz), 3.48 (1H, quin, J=6.43 Hz), 3.71-3.90 (1H, m), 4.40 (1H, t, J=6.16 Hz), 4.45 (1H, t, J=6.16 Hz), 4.54 (2H, td, J=6.50, 3.15 Hz), 4.65 (2H, dt, J=27.45, 3.79 Hz), 4.90 (1H, d, J=49.90 Hz), 4.93 (2H, dt, J=46.90, 3.79 Hz), 5.96 (1H, d, J=7.94 Hz), 7.13 (1H, d, J=3.01 Hz), 7.37 (1H, d, J=3.01 Hz), 7.88 (1H, d, J=8.49 Hz), 8.14 (1H, d, J=8.76 Hz), 8.45 (1H, s), 11.15 (1H, q, J=4.56 Hz); ESIMS found for C₂₃H₂₇F₂N₉O m/z 242.7 (M/2+1).

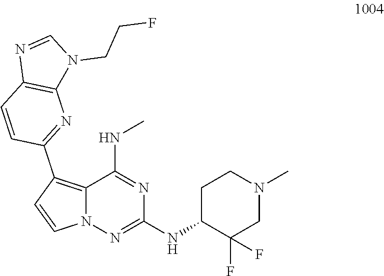 [SMILES: CNc1nc(N[C@@H]2CCN(C)CC2(F)F)nn2ccc(-c3ccc4ncn(CCF)c4n3)c12]

### (R)—N²-(3,3-Difluoro-1-methylpiperidin-4-yl)-5-(3-(2-fluoroethyl)-3H-imidazo[4,5-b]pyridin-5-yl)-N⁴-methylpyrrolo[2,1-f][1,2,4]triazine-2,4-diamine 1004

Fluffy white solid (13 mg, 0.028 mmol, 72.0% yield). ¹H NMR (499 MHz, DMSO-d₆) δ ppm 1.72-1.81 (1H, m), 1.81-1.88 (1H, m), 2.11-2.20 (1H, m), 2.25 (3H, s), 2.36 (1H, dd, J=27.15, 12.34 Hz), 2.77 (1H, br d, J=11.77 Hz), 2.97-3.08 (1H, m), 3.12 (3H, d, J=4.65 Hz), 4.14-4.31 (1H, m), 4.66 (2H, dt, J=27.40, 4.79 Hz), 4.93 (2H, dt, J=46.90, 4.79 Hz), 6.12 (1H, d, J=9.58 Hz), 7.14 (1H, d, J=3.01 Hz), 7.38 (1H, d, J=2.74 Hz), 7.89 (1H, d, J=8.76 Hz), 8.14 (1H, d, J=8.49 Hz), 8.45 (1H, s), 11.17 (1H, q, J=4.65 Hz); ESIMS found for C₂₁H₂₄F₃N₉ m/z 230.65 (M/2+1).

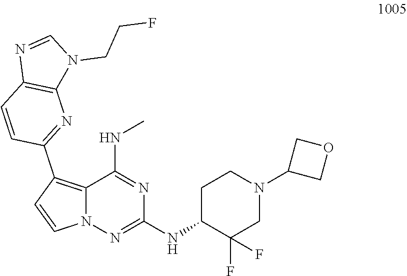 [SMILES: CNc1nc(N[C@@H]2CCN(C3COC3)CC2(F)F)nn2ccc(-c3ccc4ncn(CCF)c4n3)c12]

### (R)—N²-(3,3-Difluoro-1-(oxetan-3-yl)piperidin-4-yl)-5-(3-(2-fluoroethyl)-3H-imidazo[4,5-b]pyridin-5-yl)-N⁴-methylpyrrolo[2,1-f][1,2,4]triazine-2,4-diamine 1005

Fluffy off-white solid (e (10 mg, 0.020 mmol, 57.2% yield). ¹H NMR (499 MHz, DMSO-d₆) δ ppm 1.72-1.82 (1H, m), 1.83-1.91 (1H, m), 2.14 (1H, br t, J=10.68 Hz), 2.38 (1H, dd, J=26.30, 11.77 Hz), 2.74 (1H, br d, J=11.50 Hz), 2.95-3.05 (1H, m), 3.13 (3H, d, J=4.65 Hz), 3.60 (1H, quin, J=6.30 Hz), 4.20-4.35 (1H, m), 4.44 (2H, dt, J=14.31, 6.26 Hz), 4.55 (2H, td, J=6.64, 3.42 Hz), 4.66 (2H, dt, J=27.40, 4.79 Hz), 4.94 (2H, dt, J=46.90, 4.79 Hz), 6.21 (1H, d, J=9.31 Hz), 7.15 (1H, d, J=3.01 Hz), 7.38 (1H, d, J=2.74 Hz), 7.89 (1H, d, J=8.76 Hz), 8.14 (1H, d, J=8.49 Hz), 8.45 (1H, s), 11.18 (1H, q, J=4.65 Hz); ESIMS found for C₂₃H₂₆F₃N₉O m/z 251.70 (M/2+1).

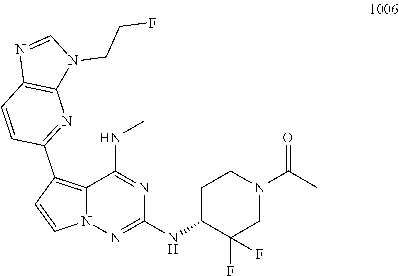 [SMILES: CNc1nc(N[C@@H]2CCN(C(C)=O)CC2(F)F)nn2ccc(-c3ccc4ncn(CCF)c4n3)c12]

### (R)-1-(3,3-Difluoro-4-((5-(3-(2-fluoroethyl)-3H-imidazo[4,5-b]pyridin-5-yl)-4-(methylamino)pyrrolo[2,1-f][1,2,4]triazin-2-yl)amino)piperidin-1-yl)ethan-1-one 1006

Fluffy white solid (7 mg, 0.014 mmol, 46.8% yield). ¹H NMR (499 MHz, DMSO-d₆) δ ppm 1.56-1.80 (1H, m), 1.82-1.99 (1H, m), 2.02-2.08 (3H, m), 3.00 (1H, br t, J=10.81 Hz), 3.13 (3H, d, J=4.65 Hz), 3.61-3.90 (1H, m), 4.08-4.28 (1H, m), 4.41-4.57 (2H, m), 4.66 (2H, dt, J=27.45, 4.79 Hz), 4.94 (2H, dt, J=47.20, 4.95 Hz), 6.35 (1H, dd, J=9.45, 6.43 Hz), 7.15 (1H, d, J=3.01 Hz), 7.38 (1H, dd, J=2.74, 1.64 Hz), 7.89 (1H, d, J=8.76 Hz), 8.14 (1H, d, J=8.76 Hz), 8.46 (1H, s), 11.20 (1H, q, J=4.38 Hz); ESIMS found for C₂₂H₂₄F₃N₉O m/z 244.65 (M/2+1).

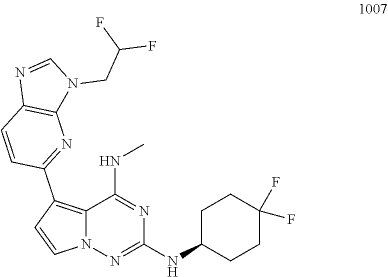

### N²-(4,4-Difluorocyclohexyl)-5-(3-(2,2-difluoroethyl)-3H-imidazo[4,5-b]pyridin-5-yl)-N⁴-methylpyrrolo[2,1-f][1,2,4]triazine-2,4-diamine 1007

Off-white solid (12 mg, 0.026 mmol, 63.0% yield). ¹H NMR (499 MHz, DMSO-d₆) δ ppm 1.55-1.69 (2H, m), 1.82-2.00 (4H, m), 2.01-2.14 (2H, m), 3.12 (3H, d, J=4.65 Hz), 3.73-3.84 (1H, m), 4.83 (2H, td, J=16.08, 2.60 Hz), 6.17 (1H, d, J=7.94 Hz), 6.62 (1H, tt, J=54.60, 3.01 Hz), 7.13 (1H, d, J=2.74 Hz), 7.37 (1H, d, J=2.74 Hz), 7.91 (1H, d, J=8.49 Hz), 8.15 (1H, d, J=8.49 Hz), 8.43 (1H, s), 10.99 (1H, q, J=4.56 Hz); ESIMS found for C₂₁H₂₂F₄N₈ m/z 232.1 (M/2+1).

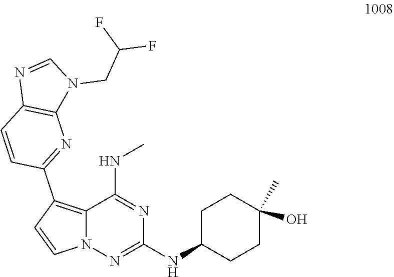 [SMILES: CNc1nc(N[C@H]2CC[C@@](C)(O)CC2)nn2ccc(-c3ccc4ncn(CC(F)F)c4n3)c12]

### (1r,4r)-4-((5-(3-(2,2-Difluoroethyl)-3H-imidazo[4,5-b]pyridin-5-yl)-4-(methylamino)pyrrolo[2,1-f][1,2,4]triazin-2-yl)amino)-1-methylcyclohexan-1-ol 1008

Off-white fluffy solid (12 mg, 0.026 mmol, 54.3% yield). ¹H NMR (499 MHz, DMSO-d₆) δ ppm 1.14 (3H, s), 1.35-1.49 (4H, m), 1.53-1.65 (2H, m), 1.78-1.90 (2H, m), 3.11 (3H, d, J=4.65 Hz), 3.62 (1H, br dd, J=8.35, 3.97 Hz), 4.21 (1H, s), 4.82 (2H, td, J=16.08, 2.60 Hz), 5.86 (1H, d, J=8.21 Hz), 6.62 (1H, tt, J=54.30, 3.00 Hz), 7.12 (1H, d, J=2.74 Hz), 7.38 (1H, d, J=3.01 Hz), 7.90 (1H, d, J=8.76 Hz), 8.14 (1H, d, J=8.76 Hz), 8.41 (1H, s), 10.95 (1H, q, J=4.38 Hz); ESIMS found for C₂₂H₂₆F₂N₈O m/z 457.2 (M+1).

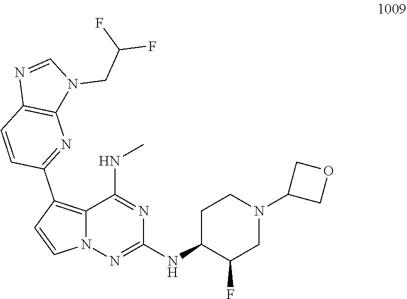 [SMILES: CNc1nc(N[C@H]2CCN(C3COC3)C[C@H]2F)nn2ccc(-c3ccc4ncn(CC(F)F)c4n3)c12]

### 5-(3-(2,2-Difluoroethyl)-3H-imidazo[4,5-b]pyridin-5-yl)-N²-((3R,4S)-3-fluoro-1-(oxetan-3-yl)piperidin-4-yl)-N4-methylpyrrolo[2,1-f][1,2,4]triazine-2,4-diamine 1009

Off-white fluffy solid (7 mg, 0.014 mmol, 33.0% yield). ¹H NMR (499 MHz, DMSO-d₆) δ ppm 1.65-1.76 (1H, m), 1.87 (1H, qd, J=12.05, 3.56 Hz), 1.95-2.04 (1H, m), 2.13 (1H, dd, J=37.30, 12.59 Hz), 2.75 (1H, br d, J=11.23 Hz), 2.92-3.03 (1H, m), 3.12 (3H, d, J=4.65 Hz), 3.49 (1H, quin, J=6.37 Hz), 3.73-3.90 (1H, m), 4.40 (1H, t, J=6.02 Hz), 4.46 (1H, t, J=6.16 Hz), 4.54 (2H, td, J=6.50, 3.15 Hz), 4.83 (2H, td, J=16.20, 2.60 Hz), 4.91 (1H, d, J=47.45 Hz), 5.97 (1H, d, J=8.21 Hz), 6.63 (1H, tt, J=54.30, 3.00 Hz), 7.15 (1H, d, J=3.01 Hz), 7.37 (1H, d, J=3.01 Hz), 7.91 (1H, d, J=8.76 Hz), 8.16 (1H, d, J=8.76 Hz), 8.42 (1H, s), 11.04 (1H, q, J=4.56 Hz); ESIMS found for C₂₃H₂₆F₃N₉O m/z 502.2 (M+1).

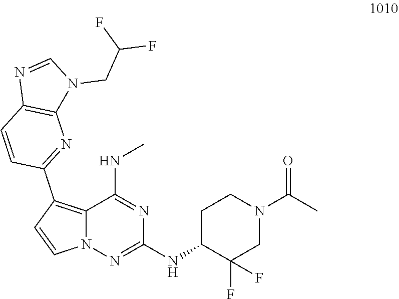 [SMILES: CNc1nc(N[C@@H]2CCN(C(C)=O)CC2(F)F)nn2ccc(-c3ccc4ncn(CC(F)F)c4n3)c12]

### (R)-1-(4-((5-(3-(2,2-Difluoroethyl)-3H-imidazo[4,5-b]pyridin-5-yl)-4-(methylamino)pyrrolo[2,1-f][1,2,4]triazin-2-yl)amino)-3,3-difluoropiperidin-1-yl)ethan-1-one 1010

White solid (14 mg, 0.028 mmol, 32.1% yield). ¹H NMR (499 MHz, DMSO-d₆) δ ppm 1.53-1.81 (1H, m), 1.82-1.98 (1H, m), 2.01-2.09 (3H, m), 2.92-3.05 (1H, m), 3.15 (3H, d, J=4.65 Hz), 3.58-3.91 (1H, m), 4.08-4.27 (1H, m), 4.41-4.61 (2H, m), 4.84 (2H, td, J=16.08, 2.60 Hz), 6.37 (1H, dd, J=9.31, 5.75 Hz), 6.63 (1H, tt, J=54.30, 3.00 Hz), 7.17 (1H, d, J=3.01 Hz), 7.38 (1H, dd, J=2.87, 1.51 Hz), 7.92 (1H, d, J=8.76 Hz), 8.16 (1H, d, J=8.76 Hz), 8.43 (1H, s), 11.09 (1H, q, J=4.56 Hz); ESIMS found for C₂₂H₂₃F₄N₉O m/z 506.2 (M+1).

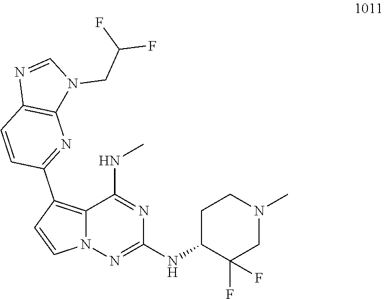 [SMILES: CNc1nc(N[C@@H]2CCN(C)CC2(F)F)nn2ccc(-c3ccc4ncn(CC(F)F)c4n3)c12]

### (R)—N²-(3,3-Difluoro-1-methylpiperidin-4-yl)-5-(3-(2,2-difluoroethyl)-3H-imidazo[4,5-b]pyridin-5-yl)-N⁴-methylpyrrolo[2,1-f][1,2,4]triazine-2,4-diamine 1011

Fluffy off-white solid (14 mg, 0.029 mmol, 74.6% yield). ¹H NMR (499 MHz, DMSO-d₆) δ ppm 1.72-1.81 (1H, m), 1.81-1.87 (1H, m), 2.16 (1H, br t, J=10.13 Hz), 2.25 (3H, s), 2.36 (1H, dd, J=27.15, 11.77 Hz), 2.77 (1H, br d, J=11.77 Hz), 2.99-3.08 (1H, m), 3.13 (3H, d, J=4.93 Hz), 4.15-4.32 (1H, m), 4.83 (2H, td, J=16.45, 2.75 Hz), 6.14 (1H, d, J=9.58 Hz), 6.63 (1H, tt, J=54.30, 3.00 Hz), 7.16 (1H, d, J=3.01 Hz), 7.39 (1H, d, J=2.74 Hz), 7.92 (1H, d, J=8.76 Hz), 8.16 (1H, d, J=8.76 Hz), 8.42 (1H, s), 11.06 (1H, q, J=4.38 Hz); ESIMS found for C₂₁H₂₃F₄N₉ m/z 239.65 (M/2+1).

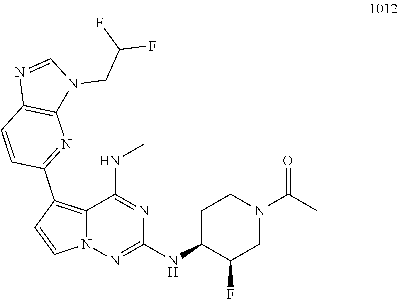 [SMILES: CNc1nc(N[C@H]2CCN(C(C)=O)C[C@H]2F)nn2ccc(-c3ccc4ncn(CC(F)F)c4n3)c12]

### 1-((3R,4S)-4-((5-(3-(2,2-Difluoroethyl)-3H-imidazo[4,5-b]pyridin-5-yl)-4-(methylamino)pyrrolo[2,1-f][1,2,4]triazin-2-yl)amino)-3-fluoropiperidin-1-yl)ethan-1-one 1012

Light yellow solid (21 mg, 0.043 mmol, 54.4% yield). ¹H NMR (499 MHz, DMSO-d₆) δ ppm 1.59-1.71 (1H, m), 1.73-1.89 (1H, m), 1.97-2.06 (3H, m), 2.66-2.95 (1H, m), 3.13 (3H, d, J=4.65 Hz), 3.84-3.92 (1H, m), 3.93-4.06 (1H, m), 4.07-4.17 (1H, m), 4.37-4.73 (1H, m), 4.83 (2H, td, J=16.29, 2.74 Hz), 4.97 (1H, d, J=49.65 Hz), 6.11 (1H, dd, J=8.08, 3.70 Hz), 6.63 (1H, tt, J=54.55, 3.05 Hz), 7.16 (1H, d, J=3.01 Hz), 7.38 (1H, d, J=3.01 Hz), 7.92 (1H, d, J=8.76 Hz), 8.16 (1H, d, J=8.49 Hz), 8.43 (1H, s), 11.05 (1H, q, J=4.38 Hz); ESIMS found for C₂₂H₂₄F₃N₉O m/z 488.2 M+1

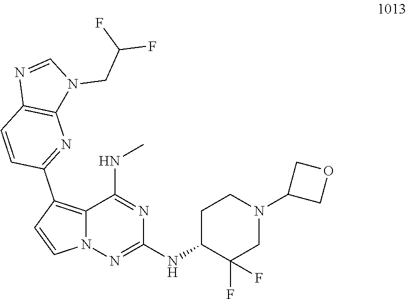 [SMILES: CNc1nc(N[C@@H]2CCN(C3COC3)CC2(F)F)nn2ccc(-c3ccc4ncn(CC(F)F)c4n3)c12]

### (R)—N²-(3,3-Difluoro-1-(oxetan-3-yl)piperidin-4-yl)-5-(3-(2,2-difluoroethyl)-3H-imidazo[4,5-b]pyridin-5-yl)-N4-methylpyrrolo[2,1-f][1,2,4]triazine-2,4-diamine 1013

Yellowish white solid (5 mg, 0.010 mmol, 11.2% yield). ¹H NMR (499 MHz, DMSO-d₆) δ ppm 1.70-1.82 (1H, m), 1.85-1.92 (1H, m), 2.09-2.20 (1H, m), 2.32-2.44 (1H, m), 2.74 (1H, br d, J=11.50 Hz), 2.93-3.04 (1H, m), 3.14 (3H, d, J=4.65 Hz), 3.60 (1H, quin, J=6.30 Hz), 4.22-4.36 (1H, m), 4.44 (2H, dt, J=14.44, 6.19 Hz), 4.55 (2H, td, J=6.57, 3.29 Hz), 4.75-4.90 (2H, m), 6.23 (1H, d, J=9.58 Hz), 6.63 (1H, tt, J=54.30, 3.00 Hz), 7.16 (1H, d, J=3.01 Hz), 7.38 (1H, d, J=3.01 Hz), 7.92 (1H, d, J=8.76 Hz), 8.16 (1H, d, J=8.49 Hz), 8.41-8.45 (1H, m), 11.04-11.12 (1H, m); ESIMS found for C₂₃H₂₅F₄N₉O m/z 520.2 (M+1).

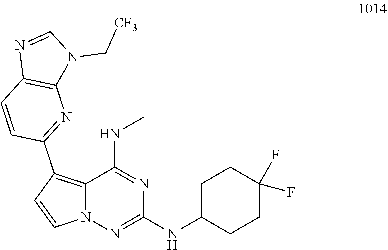 [SMILES: CNc1nc(NC2CCC(F)(F)CC2)nn2ccc(-c3ccc4ncn(CC(F)(F)F)c4n3)c12]

### N²-(4,4-Difluorocyclohexyl)-N⁴-methyl-5-(3-(2,2,2-trifluoroethyl)-3H-imidazo[4,5-b]pyridin-5-yl)pyrrolo[2,1-f][1,2,4]triazine-2,4-diamine 1014

Fluffy white solid (11 mg, 0.023 mmol, 55.6% yield). ¹H NMR (499 MHz, DMSO-d₆) δ ppm 1.53-1.68 (2H, m), 1.81-2.00 (4H, m), 2.01-2.14 (2H, m), 3.11 (3H, d, J=4.65 Hz), 3.70-3.85 (1H, m), 5.35 (2H, q, J=9.49 Hz), 6.21 (1H, d, J=7.94 Hz), 7.16 (1H, d, J=3.01 Hz), 7.38 (1H, d, J=2.74 Hz), 7.96 (1H, d, J=8.76 Hz), 8.18 (1H, d, J=8.76 Hz), 8.49 (1H, s), 10.91 (1H, q, J=4.38 Hz); ESIMS found for C₂₁H₂₁F₅N₈ m/z 481.2 (M+1).

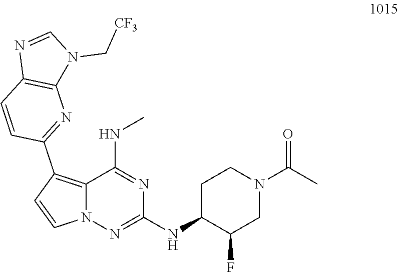 [SMILES: CNc1nc(N[C@H]2CCN(C(C)=O)C[C@H]2F)nn2ccc(-c3ccc4ncn(CC(F)(F)F)c4n3)c12]

### 1-((3R,4S)-3-Fluoro-4-((4-(methylamino)-5-(3-(2,2,2-trifluoroethyl)-3H-imidazo[4,5-b]pyridin-5-yl)pyrrolo[2,1-f][1,2,4]triazin-2-yl)amino)piperidin-1-yl)ethan-1-one 1015

Fluffy off-white solid (15 mg, 0.030 mmol, 77.8% yield). ¹H NMR (499 MHz, DMSO-d₆) δ ppm 1.61-1.71 (1H, m), 1.72-1.86 (1H, m), 1.98-2.05 (3H, m), 2.66-2.96 (1H, m), 3.12 (3H, d, J=4.65 Hz), 3.83-3.92 (1H, m), 3.92-4.05 (1H, m), 4.06-4.17 (1H, m), 4.39-4.76 (1H, m), 4.97 (1H, d, J=49.35 Hz), 5.36 (2H, q, J=9.22 Hz), 6.15 (1H, dd, J=7.94, 3.29 Hz), 7.19 (1H, d, J=3.01 Hz), 7.39 (1H, d, J=2.74 Hz), 7.97 (1H, d, J=8.76 Hz), 8.19 (1H, d, J=8.76 Hz), 8.49 (1H, s), 10.97 (1H, q, J=4.56 Hz); ESIMS found for C₂₂H₂₃F₄N₉O m/z 506.2 (M+1).

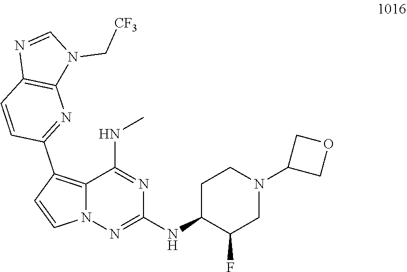 [SMILES: CNc1nc(N[C@H]2CCN(C3COC3)C[C@H]2F)nn2ccc(-c3ccc4ncn(CC(F)(F)F)c4n3)c12]

### N²-((3R,4S)-3-Fluoro-1-(oxetan-3-yl)piperidin-4-yl)-N⁴-methyl-5-(3-(2,2,2-trifluoroethyl)-3H-imidazo[4,5-b]pyridin-5-yl)pyrrolo[2,1-f][1,2,4]triazine-2,4-diamine 1016

Fluffy off-white solid (15 mg, 0.029 mmol, 78.8% yield). ¹HNMR (499 MHz, DMSO-d₆) δ ppm 1.65-1.76 (1H, m), 1.88 (1H, qd, J=12.14, 3.83 Hz), 1.95-2.05 (1H, m), 2.13 (1H, dd, J=37.60, 12.87 Hz), 2.74 (1H, br d, J=10.40 Hz), 2.91-3.03 (1H, m), 3.12 (3H, d, J=4.65 Hz), 3.48 (1H, quin, J=6.37 Hz), 3.72-3.90 (1H, m), 4.40 (1H, t, J=6.16 Hz), 4.46 (1H, t, J=6.16 Hz), 4.54 (2H, td, J=6.50, 3.15 Hz), 4.90 (1H, d, J=49.90 Hz), 5.35 (2H, q, J=9.22 Hz), 6.02 (1H, d, J=7.94 Hz), 7.18 (1H, d, J=3.01 Hz), 7.39 (1H, d, J=2.74 Hz), 7.96 (1H, d, J=8.76 Hz), 8.19 (1H, d, J=8.76 Hz), 8.49 (1H, s), 10.95 (1H, q, J=4.56 Hz); ESIMS found for C₂₃H₂₅F₄N₉O m/z 260.70 (M/2+1).

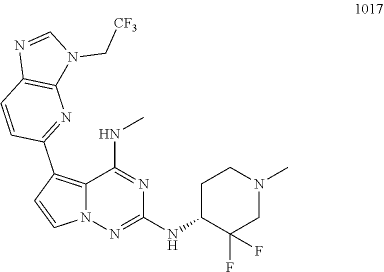 [SMILES: CNc1nc(N[C@@H]2CCN(C)CC2(F)F)nn2ccc(-c3ccc4ncn(CC(F)(F)F)c4n3)c12]

### (R)—N²-(3,3-Difluoro-1-methylpiperidin-4-yl)-N⁴-methyl-5-(3-(2,2,2-trifluoroethyl)-3H-imidazo[4,5-b]pyridin-5-yl)pyrrolo[2,1-f][1,2,4]triazine-2,4-diamine 1017

Fluffy off-white solid (13 mg, 0.026 mmol, 66.8% yield). ¹H NMR (499 MHz, DMSO-d₆) δ ppm 1.69-1.90 (2H, m), 2.08-2.19 (1H, m), 2.25 (3H, s), 2.35 (1H, dd, J=26.90, 12.32 Hz), 2.71-2.82 (1H, m), 2.97-3.08 (1H, m), 3.13 (3H, d, J=4.65 Hz), 4.14-4.31 (1H, m), 5.36 (2H, q, J=9.22 Hz), 6.18 (1H, d, J=9.58 Hz), 7.19 (1H, d, J=3.01 Hz), 7.40 (1H, d, J=2.74 Hz), 7.97 (1H, d, J=8.76 Hz), 8.19 (1H, d, J=8.76 Hz), 8.49 (1H, s), 10.97 (1H, q, J=4.65 Hz); ESIMS found for C₂₁H₂₂F₅N₉ m/z 248.65 (M/2+1).

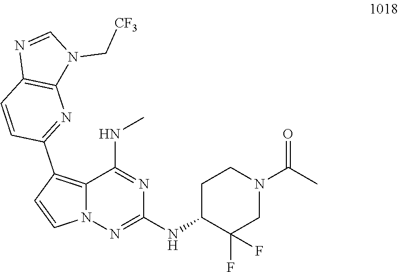 [SMILES: CNc1nc(N[C@@H]2CCN(C(C)=O)CC2(F)F)nn2ccc(-c3ccc4ncn(CC(F)(F)F)c4n3)c12]

### (R)-1-(3,3-Difluoro-4-((4-(methylamino)-5-(3-(2,2,2-trifluoroethyl)-3H-imidazo[4,5-b]pyridin-5-yl)pyrrolo[2,1-f][1,2,4]triazin-2-yl)amino)piperidin-1-yl)ethan-1-one 1018

Fluffy off-white solid (15 mg, 0.029 mmol, 79.1% yield). ¹H NMR (499 MHz, DMSO-d₆) δ ppm 1.55-1.80 (1H, m), 1.82-1.98 (1H, m), 2.02-2.08 (3H, m), 2.99 (1H, br t, J=10.95 Hz), 3.14 (3H, d, J=4.65 Hz), 3.62-3.89 (1H, m), 4.09-4.28 (1H, m), 4.42-4.60 (2H, m), 5.36 (2H, q, J=9.22 Hz), 6.41 (1H, dd, J=9.31, 5.48 Hz), 7.20 (1H, d, J=3.01 Hz), 7.39 (1H, dd, J=2.74, 1.64 Hz), 7.98 (1H, d, J=8.76 Hz), 8.20 (1H, d, J=8.49 Hz), 8.50 (1H, s), 11.00 (1H, q, J=4.56 Hz); ESIMS found for C₂₂H₂₂F₅N₉O m/z 524.2 (M+1).

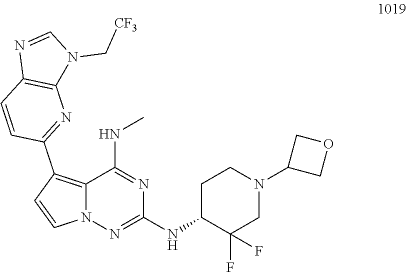 [SMILES: CNc1nc(N[C@@H]2CCN(C3COC3)CC2(F)F)nn2ccc(-c3ccc4ncn(CC(F)(F)F)c4n3)c12]

### (R)—N²-(3,3-Difluoro-1-(oxetan-3-yl)piperidin-4-yl)-N⁴-methyl-5-(3-(2,2,2-trifluoroethyl)-3H-imidazo[4,5-b]pyridin-5-yl)pyrrolo[2,1-f][1,2,4]triazine-2,4-diamine 1019

Fluffy off-white solid (4 mg, 0.007 mmol, 21.3% yield). ¹H NMR (499 MHz, DMSO-d₆) δ ppm 1.73-1.82 (1H, m), 1.84-1.91 (1H, m), 2.14 (1H, br t, J=10.68 Hz), 2.38 (1H, dd, J=26.85, 11.77 Hz), 2.74 (1H, br d, J=11.23 Hz), 2.95-3.05 (1H, m), 3.13 (3H, d, J=4.65 Hz), 3.60 (1H, quin, J=6.23 Hz), 4.20-4.36 (1H, m), 4.44 (2H, dt, J=14.44, 6.19 Hz), 4.55 (2H, td, J=6.64, 3.42 Hz), 5.36 (2H, q, J=9.13 Hz), 6.26 (1H, d, J=9.31 Hz), 7.19 (1H, d, J=3.01 Hz), 7.39 (1H, d, J=3.01 Hz), 7.97 (1H, d, J=8.76 Hz), 8.19 (1H, d, J=8.49 Hz), 8.49 (1H, s), 10.98 (1H, q, J=4.38 Hz); ESIMS found for C₂₃H₂₄F₅N₉O m/z 269.65 (M/2+1).

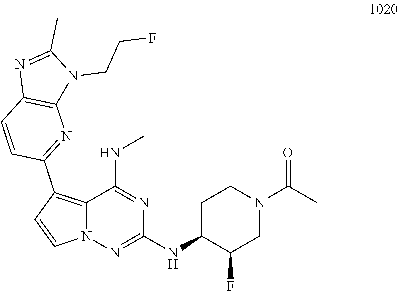 [SMILES: CNc1nc(N[C@H]2CCN(C(C)=O)C[C@H]2F)nn2ccc(-c3ccc4nc(C)n(CCF)c4n3)c12]

### 1-((3R,4S)-3-Fluoro-4-((5-(3-(2-fluoroethyl)-2-methyl-3H-imidazo[4,5-b]pyridin-5-yl)-4-(methylamino)pyrrolo[2,1-f][1,2,4]triazin-2-yl)amino)piperidin-1-yl)ethan-1-one 1020

White solid (13 mg, 0.027 mmol, 53.9% yield). ¹H NMR (499 MHz, DMSO-d₆) δ ppm 1.56-1.72 (1H, m), 1.72-1.87 (1H, m), 1.98-2.07 (3H, m), 2.61 (3H, s), 2.78-2.96 (1H, m), 3.11 (3H, d, J=4.65 Hz), 3.84-3.92 (1H, m), 3.93-4.06 (1H, m), 4.07-4.16 (1H, m), 4.34-4.49 (1H, m), 4.61 (2H, dt, J=26.60, 4.79 Hz), 4.90 (2H, dt, J=46.90, 4.93 Hz), 4.97 (1H, d, J=49.65 Hz), 6.06 (1H, dd, J=8.08, 4.79 Hz), 7.10 (1H, d, J=3.01 Hz), 7.36 (1H, d, J=2.74 Hz), 7.82 (1H, d, J=8.49 Hz), 7.99 (1H, d, J=8.49 Hz), 11.13 (1H, q, J=4.47 Hz); ESIMS found for C₂₃H₂₇F₂N₉O m/z 484.2 (M+1).

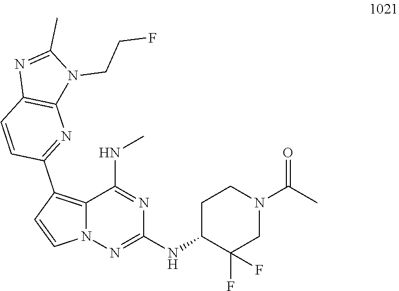 [SMILES: CNc1nc(N[C@@H]2CCN(C(C)=O)CC2(F)F)nn2ccc(-c3ccc4nc(C)n(CCF)c4n3)c12]

### (R)-1-(3,3-Difluoro-4-((5-(3-(2-fluoroethyl)-2-methyl-3H-imidazo[4,5-b]pyridin-5-yl)-4-(methylamino)pyrrolo[2,1-f][1,2,4]triazin-2-yl)amino)piperidin-1-yl)ethan-1-one 1021

White solid (11 mg, 0.022 mmol, 43.7% yield). ¹H NMR (499 MHz, DMSO-d₆) δ ppm 1.54-1.80 (1H, m), 1.82-2.00 (1H, m), 2.02-2.09 (3H, m), 2.62 (3H, s), 3.00 (1H, br t, J=10.95 Hz), 3.13 (3H, d, J=4.93 Hz), 3.60-3.90 (1H, m), 4.08-4.27 (1H, m), 4.40-4.55 (2H, m), 4.61 (2H, dt, J=26.90, 4.79 Hz), 4.90 (2H, dt, J=46.65, 4.93 Hz), 6.33 (1H, dd, J=9.45, 6.71 Hz), 7.11 (1H, d, J=2.74 Hz), 7.36 (1H, dd, J=2.74, 1.64 Hz), 7.82 (1H, d, J=8.49 Hz), 7.99 (1H, d, J=8.49 Hz), 11.16 (1H, q, J=4.56 Hz); ESIMS found for C₂₃H₂₆F₃N₉O m/z 502.2 (M+1).

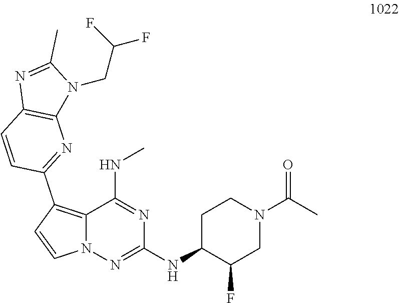 [SMILES: CNc1nc(N[C@H]2CCN(C(C)=O)C[C@H]2F)nn2ccc(-c3ccc4nc(C)n(CC(F)F)c4n3)c12]

### 1-((3R,4S)-4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-3-fluoropiperidin-1-yl)ethan-1-one 1022

White solid (17 mg, 0.034 mmol, 68.0% yield). ¹H NMR (499 MHz, DMSO-d₆) δ ppm 1.58-1.71 (1H, m), 1.73-1.87 (1H, m), 1.97-2.06 (3H, m), 2.80-2.96 (1H, m), 3.13 (3H, d, J=4.93 Hz), 3.89 (1H, br d, J=13.69 Hz), 3.93-4.06 (1H, m), 4.11 (1H, ddd, J=12.05, 9.86, 2.46 Hz), 4.39-4.71 (1H, m), 4.78 (2H, td, J=16.02, 2.46 Hz), 4.97 (1H, d, J=49.40 Hz), 6.08 (1H, dd, J=8.08, 4.24 Hz), 6.64 (1H, tt, J=54.60, 3.00 Hz), 7.11 (1H, d, J=3.01 Hz), 7.37 (1H, d, J=3.01 Hz), 7.85 (1H, d, J=8.49 Hz), 8.01 (1H, d, J=8.49 Hz), 10.99 (1H, q, J=4.65 Hz); ESIMS found for C₂₃H₂₆F₃N₉O m/z 502.2 (M+1).

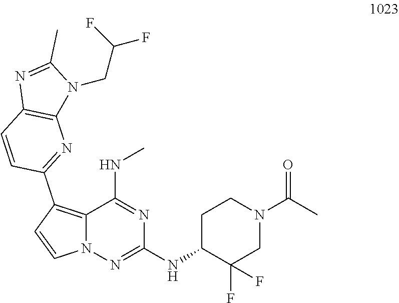 [SMILES: CNc1nc(N[C@@H]2CCN(C(C)=O)CC2(F)F)nn2ccc(-c3ccc4nc(C)n(CC(F)F)c4n3)c12]

### (R)-1-(4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-3,3-difluoropiperidin-1-yl)ethan-1-one 1023

White solid (11 mg, 0.021 mmol, 42.2% yield). ¹H NMR (499 MHz, DMSO-d₆) δ ppm 1.54-1.80 (1H, m), 1.82-1.98 (1H, m), 2.03-2.08 (3H, m), 2.63 (3H, s), 2.95-3.05 (1H, m), 3.15 (3H, d, J=4.65 Hz), 3.62-3.90 (1H, m), 4.08-4.28 (1H, m), 4.42-4.60 (2H, m), 4.78 (2H, td, J=15.88, 2.46 Hz), 6.34 (1H, dd, J=9.45, 6.43 Hz), 6.64 (1H, tt, J=54.30, 3.05 Hz), 7.12 (1H, d, J=3.01 Hz), 7.37 (1H, dd, J=2.74, 1.64 Hz), 7.86 (1H, d, J=8.49 Hz), 8.01 (1H, d, J=8.49 Hz), 11.02 (1H, q, J=4.65 Hz); ESIMS found for C₂₃H₂₅F₄N₉O m/z 520 (M+1).

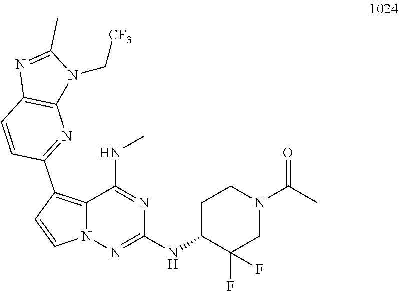 [SMILES: CNc1nc(N[C@@H]2CCN(C(C)=O)CC2(F)F)nn2ccc(-c3ccc4nc(C)n(CC(F)(F)F)c4n3)c12]

### (R)-1-(3,3-Difluoro-4-((5-(2-methyl-3-(2,2,2-trifluoroethyl)-3H-imidazo[4,5-b]pyridin-5-yl)-4-(methylamino)pyrrolo[2,1-f][1,2,4]triazin-2-yl)amino)piperidin-1-yl)ethan-1-one 1024

White solid (17 mg, 0.032 mmol, 63.0% yield). ¹H NMR (499 MHz, DMSO-d₆) δ ppm 1.55-1.78 (1H, m), 1.81-1.98 (1H, m), 2.02-2.11 (3H, m), 2.64 (3H, s), 2.94-3.06 (1H, m), 3.14 (3H, d, J=4.65 Hz), 3.60-3.91 (1H, m), 4.08-4.28 (1H, m), 4.41-4.59 (2H, m), 5.30 (2H, q, J=9.22 Hz), 6.38 (1H, dd, J=9.58, 5.75 Hz), 7.15 (1H, d, J=3.01 Hz), 7.38 (1H, dd, J=2.74, 1.64 Hz), 7.91 (1H, d, J=8.76 Hz), 8.05 (1H, d, J=8.49 Hz), 10.92 (1H, q, J=4.47 Hz); ESIMS found for C₂₃H₂₄F₅N₉O m/z 538.2 (M+1).

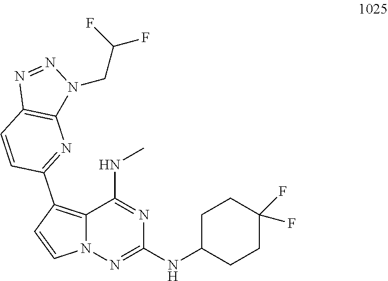 [SMILES: CNc1nc(NC2CCC(F)(F)CC2)nn2ccc(-c3ccc4nnn(CC(F)F)c4n3)c12]

### N²-(4,4-Difluorocyclohexyl)-5-(3-(2,2-difluoroethyl)-3H-[1,2,3]triazolo[4,5-b]pyridin-5-yl)-N⁴-methylpyrrolo[2,1-f][1,2,4]triazine-2,4-diamine 1025

Fluffy yellow solid (16 mg, 0.035 mmol, 83.9% yield). ¹H NMR (499 MHz, DMSO-d₆) δ ppm 1.56-1.69 (2H, m), 1.83-2.00 (4H, m), 2.02-2.14 (2H, m), 3.13 (3H, d, J=4.65 Hz), 3.72-3.86 (1H, m), 5.26 (2H, td, J=16.08, 2.60 Hz), 6.33 (1H, d, J=7.94 Hz), 6.72 (2H, tt, J=54.05, 3.01 Hz), 7.33 (1H, d, J=3.01 Hz), 7.43 (1H, d, J=3.01 Hz), 8.11 (1H, d, J=9.04 Hz), 8.55 (1H, d, J=8.76 Hz), 10.89 (1H, q, J=4.38 Hz); ESIMS found for C₂₀H₂₁F₄N₉ m/z 464.2 (M+1).

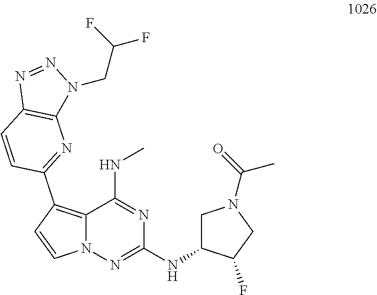 [SMILES: CNc1nc(N[C@@H]2CN(C(C)=O)C[C@@H]2F)nn2ccc(-c3ccc4nnn(CC(F)F)c4n3)c12]

### 1-((3R,4S)-3-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-4-fluoropyrrolidin-1-yl)ethan-1-one 1026

Yellow solid (23 mg, 0.049 mmol, 46.6% yield). ¹H NMR (499 MHz, DMSO-d₆) δ ppm 1.92-2.00 (3H, m), 3.15 (3H, d, J=4.93 Hz), 3.44-3.55 (1H, m), 3.60-3.74 (1H, m), 3.77-3.85 (1H, m), 3.86-3.95 (1H, m), 4.29-4.61 (1H, m), 5.21-5.44 (1H, m), 5.27 (2H, td, J=16.45, 2.75 Hz), 6.62 (1H, s), 6.73 (1H, tt, J=54.30, 3.00 Hz), 7.36-7.38 (1H, m), 7.45-7.48 (1H, m), 8.13 (1H, d, J=9.31 Hz), 8.53-8.60 (1H, m), 10.96-11.02 (1H, m); ESIMS found for C₂₀H₂₁F₃N₁₀O m/z 475.2 (M+1).

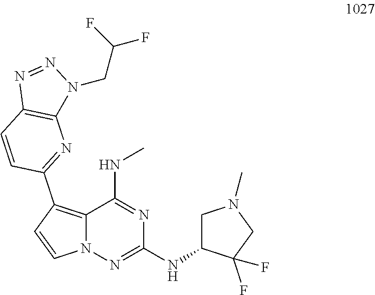 [SMILES: CNc1nc(N[C@@H]2CN(C)CC2(F)F)nn2ccc(-c3ccc4nnn(CC(F)F)c4n3)c12]

### (R)—N²-(4,4-Difluoro-1-methylpyrrolidin-3-yl)-5-(3-(2,2-difluoroethyl)-3H-[1,2,3]triazolo[4,5-b]pyridin-5-yl)-N⁴-methylpyrrolo[2,1-f][1,2,4]triazine-2,4-diamine 1027

Yellow solid (11 mg, 0.024 mmol, 53.3% yield). ¹H NMR (499 MHz, DMSO-d₆) δ ppm 2.27 (3H, s), 2.40 (1H, t, J=9.31 Hz), 2.52-2.61 (1H, m), 3.14 (3H, d, J=4.93 Hz), 3.20-3.26 (2H, m), 4.56-4.69 (1H, m), 5.27 (2H, td, J=16.15, 2.74 Hz), 6.57 (1H, d, J=8.76 Hz), 6.72 (1H, tt, J=54.00, 3.00 Hz), 7.36 (1H, d, J=3.29 Hz), 7.44-7.49 (1H, m), 8.12 (1H, d, J=9.31 Hz), 8.56 (1H, d, J=8.76 Hz), 10.97 (1H, q, J=4.75 Hz); ESIMS found for C₁₉H₂₀F₄N₁₀ m/z 465.2 (M+1).

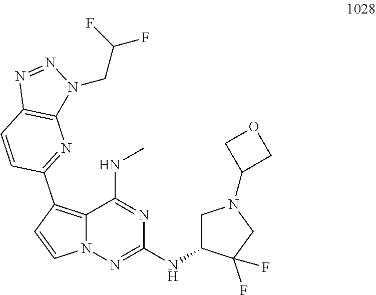 [SMILES: CNc1nc(N[C@@H]2CN(C3COC3)CC2(F)F)nn2ccc(-c3ccc4nnn(CC(F)F)c4n3)c12]

### (R)—N²-(4,4-Difluoro-1-(oxetan-3-yl)pyrrolidin-3-yl)-5-(3-(2,2-difluoroethyl)-3H-[1,2,3]triazolo[4,5-b]pyridin-5-yl)-N⁴-methylpyrrolo[2,1-f][1,2,4]triazine-2,4-diamine 1028

Yellow solid (15 mg, 0.030 mmol, 66.7% yield). ¹H NMR (499 MHz, DMSO-d₆) δ ppm 2.61-2.77 (1H, m), 3.15 (3H, d, J=4.93 Hz), 3.21-3.30 (3H, m), 3.64-3.73 (1H, m), 4.44-4.52 (2H, m), 4.58 (2H, q, J=6.94 Hz), 4.62-4.74 (1H, m), 5.27 (2H, td, J=16.02, 2.46 Hz), 6.61 (1H, d, J=9.31 Hz), 6.72 (1H, tt, J=54.00, 3.00 Hz), 7.37 (1H, d, J=3.29 Hz), 7.46 (1H, d, J=2.74 Hz), 8.13 (1H, d, J=8.76 Hz), 8.56 (1H, d, J=8.76 Hz), 10.98 (1H, q, J=4.38 Hz); ESIMS found for C₂₁H₂₂F₄N₁₀O m/z 507.2 (M+1).

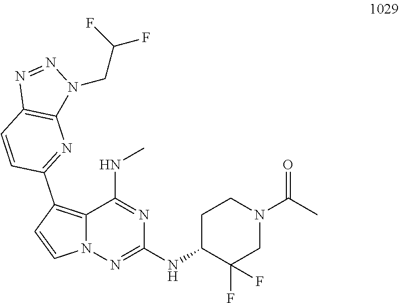 [SMILES: CNc1nc(N[C@@H]2CCN(C(C)=O)CC2(F)F)nn2ccc(-c3ccc4nnn(CC(F)F)c4n3)c12]

### (R)-1-(4-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-3,3-difluoropiperidin-1-yl)ethan-1-one 1029

Fluffy yellow solid (12 mg, 0.024 mmol, 42.5% yield). ¹H NMR (499 MHz, DMSO-d₆) δ ppm 1.55-1.82 (1H, m), 1.82-1.98 (1H, m), 2.02-2.09 (3H, m), 2.94-3.06 (1H, m), 3.16 (3H, d, J=4.93 Hz), 3.62-3.90 (1H, m), 4.09-4.28 (1H, m), 4.41-4.61 (2H, m), 5.27 (2H, td, J=16.08, 2.33 Hz), 6.54 (1H, dd, J=9.31, 4.11 Hz), 6.72 (1H, tt, J=54.05, 2.75 Hz), 7.36 (1H, d, J=3.29 Hz), 7.44 (1H, dd, J=3.01, 1.64 Hz), 8.13 (1H, d, J=9.03 Hz), 8.56 (1H, d, J=9.03 Hz), 10.92-11.02 (1H, m); ESIMS found for C₂₁H₂₂F₄N₁₀O m/z 507.2 (M+1).

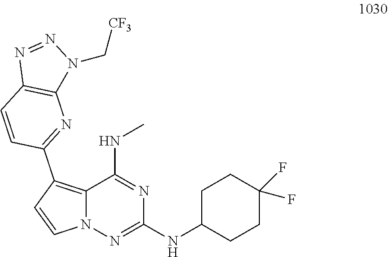 [SMILES: CNc1nc(NC2CCC(F)(F)CC2)nn2ccc(-c3ccc4nnn(CC(F)(F)F)c4n3)c12]

### N²-(4,4-Difluorocyclohexyl)-N⁴-methyl-5-(3-(2,2,2-trifluoroethyl)-3H-[1,2,3]triazolo[4,5-b]pyridin-5-yl)pyrrolo[2,1-f][1,2,4]triazine-2,4-diamine 1030

Fluffy yellow solid (11 mg, 0.023 mmol, 55.5% yield). ¹H NMR (499 MHz, DMSO-d₆) δ ppm 1.55-1.69 (2H, m), 1.83-2.00 (4H, m), 2.02-2.14 (2H, m), 3.12 (3H, d, J=4.65 Hz), 3.74-3.85 (1H, m), 5.86 (2H, q, J=9.22 Hz), 6.37 (1H, d, J=7.67 Hz), 7.36 (1H, d, J=3.29 Hz), 7.45 (1H, d, J=3.01 Hz), 8.15 (1H, d, J=9.04 Hz), 8.58 (1H, d, J=9.04 Hz), 10.80 (1H, q, J=4.47 Hz); ESIMS found for C₂₀H₂₀F₅N₉ m/z 482.2 (M+1).

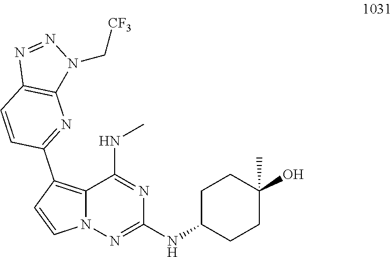 [SMILES: CNc1nc(N[C@H]2CC[C@](C)(O)CC2)nn2ccc(-c3ccc4nnn(CC(F)(F)F)c4n3)c12]

### (1r,4r)-1-Methyl-4-((4-(methylamino)-5-(3-(2,2,2-trifluoroethyl)-3H-[1,2,3]triazolo[4,5-b]pyridin-5-yl)pyrrolo[2,1-f][1,2,4]triazin-2-yl)amino)cyclohexan-1-ol 1031

Fluffy yellow solid (5 mg, 0.011 mmol, 21.7% yield). ¹H NMR (499 MHz, DMSO-d₆) δ ppm 1.14 (3H, s), 1.35-1.48 (4H, m), 1.59 (2H, br dd, J=8.35, 5.34 Hz), 1.78-1.90 (2H, m), 3.11 (3H, d, J=4.65 Hz), 3.58-3.71 (1H, m), 4.22 (1H, s), 5.85 (2H, q, J=9.31 Hz), 6.08 (1H, d, J=7.94 Hz), 7.34 (1H, d, J=3.29 Hz), 7.45 (1H, d, J=3.01 Hz), 8.14 (1H, d, J=9.31 Hz), 8.57 (1H, d, J=9.03 Hz), 10.77 (1H, q, J=4.20 Hz); ESIMS found for C₂₁H₂₄F₃N₉O m/z 476.2 (M+1).

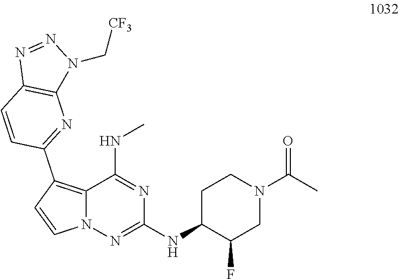 [SMILES: CNc1nc(N[C@H]2CCN(C(C)=O)C[C@H]2F)nn2ccc(-c3ccc4nnn(CC(F)(F)F)c4n3)c12]

### 1-((3R,4S)-3-Fluoro-4-((4-(methylamino)-5-(3-(2,2,2-trifluoroethyl)-3H-[1,2,3]triazolo[4,5-b]pyridin-5-yl)pyrrolo[2,1-f][1,2,4]triazin-2-yl)amino)piperidin-1-yl)ethan-1-one 1032

Fluffy yellow solid (11 mg, 0.022 mmol, 56.9% yield). ¹H NMR (499 MHz, DMSO-d₆) δ ppm 1.62-1.70 (1H, m), 1.72-1.88 (1H, m), 1.98-2.05 (3H, m), 2.66-2.96 (1H, m), 3.13 (3H, d, J=4.65 Hz), 3.83-3.92 (1H, m), 3.93-4.07 (1H, m), 4.07-4.18 (1H, m), 4.38-4.75 (1H, m), 4.97 (1H, d, J=49.35 Hz), 5.86 (2H, q, J=9.22 Hz), 6.33 (1H, d, J=7.94 Hz), 7.38 (1H, d, J=3.29 Hz), 7.45 (1H, d, J=3.01 Hz), 8.16 (1H, d, J=9.31 Hz), 8.59 (1H, d, J=9.03 Hz), 10.85 (1H, q, J=4.65 Hz); ESIMS found for C₂₁H₂₂F₄N₁₀O m/z 507.2 (M+1).

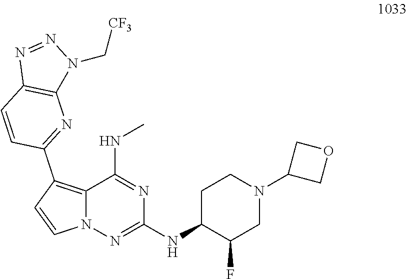 [SMILES: CNc1nc(N[C@H]2CCN(C3COC3)C[C@H]2F)nn2ccc(-c3ccc4nnn(CC(F)(F)F)c4n3)c12]

### N²-((3R,4S)-3-Fluoro-1-(oxetan-3-yl)piperidin-4-yl)-N⁴-methyl-5-(3-(2,2,2-trifluoroethyl)-3H-[1,2,3]triazolo[4,5-b]pyridin-5-yl)pyrrolo[2,1-f][1,2,4]triazine-2,4-diamine 1033

Fluffy yellow solid (6 mg, 0.012 mmol, 31.5% yield). ¹H NMR (499 MHz, DMSO-d₆) δ ppm 1.65-1.75 (1H, m), 1.89 (1H, qd, J=12.05, 3.83 Hz), 1.95-2.04 (1H, m), 2.13 (1H, dd, J=37.30, 12.59 Hz), 2.75 (1H, br d, J=9.86 Hz), 2.93-3.04 (1H, m), 3.12 (3H, d, J=4.93 Hz), 3.44-3.53 (1H, m), 3.74-3.90 (1H, m), 4.40 (1H, t, J=6.16 Hz), 4.46 (1H, t, J=6.16 Hz), 4.54 (2H, td, J=6.43, 3.01 Hz), 4.90 (1H, d, J=49.60 Hz), 5.86 (2H, q, J=9.31 Hz), 6.21 (1H, d, J=7.94 Hz), 7.37 (1H, d, J=3.29 Hz), 7.45 (1H, d, J=3.01 Hz), 8.16 (1H, d, J=9.31 Hz), 8.59 (1H, d, J=9.03 Hz), 10.84 (1H, q, J=4.65 Hz); ESIMS found for C₂₂H₂₄F₄N₁₀O m/z 521.2 (M+1).

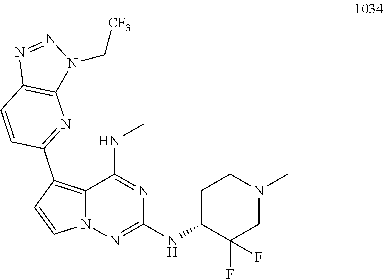 [SMILES: CNc1nc(N[C@@H]2CCN(C)CC2(F)F)nn2ccc(-c3ccc4nnn(CC(F)(F)F)c4n3)c12]

### (R)—N²-(3,3-Difluoro-1-methylpiperidin-4-yl)-N⁴-methyl-5-(3-(2,2,2-trifluoroethyl)-3H-[1,2,3]triazolo[4,5-b]pyridin-5-yl)pyrrolo[2,1-f][1,2,4]triazine-2,4-diamine 1034

Fluffy yellow solid (7 mg, 0.014 mmol, 35.9% yield). ¹H NMR (499 MHz, DMSO-d₆) δ ppm 1.70-1.87 (2H, m), 2.10-2.19 (1H, m), 2.25 (3H, s), 2.30-2.41 (1H, m), 2.77 (1H, br d, J=11.77 Hz), 2.95-3.08 (1H, m), 3.13 (3H, d, J=4.65 Hz), 4.15-4.31 (1H, m), 5.86 (2H, q, J=9.22 Hz), 6.37 (1H, d, J=9.58 Hz), 7.38 (1H, d, J=3.01 Hz), 7.46 (1H, d, J=3.01 Hz), 8.16 (1H, d, J=9.31 Hz), 8.59 (1H, d, J=9.03 Hz), 10.85 (1H, q, J=4.56 Hz); ESIMS found for C₂₀H₂₁F₅N₁₀ m/z 497.2 (M+1).

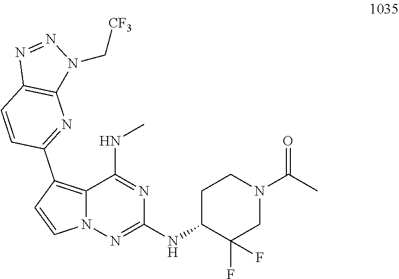 [SMILES: CNc1nc(N[C@@H]2CCN(C(C)=O)CC2(F)F)nn2ccc(-c3ccc4nnn(CC(F)(F)F)c4n3)c12]

### (R)-1-(3,3-Difluoro-4-((4-(methylamino)-5-(3-(2,2,2-trifluoroethyl)-3H-[1,2,3]triazolo[4,5-b]pyridin-5-yl)pyrrolo[2,1-f][1,2,4]triazin-2-yl)amino)piperidin-1-yl)ethan-1-one 1035

Fluffy yellow solid (7 mg, 0.013 mmol, 39.9% yield). ¹H NMR (499 MHz, DMSO-d₆) δ ppm 1.54-1.81 (1H, m), 1.82-1.98 (1H, m), 2.02-2.09 (3H, m), 2.91-3.06 (1H, m), 3.15 (3H, d, J=4.65 Hz), 3.61-3.92 (1H, m), 4.10-4.30 (1H, m), 4.42-4.61 (2H, m), 5.87 (2H, q, J=9.22 Hz), 6.58 (1H, dd, J=9.58, 3.29 Hz), 7.39 (1H, d, J=3.29 Hz), 7.46 (1H, dd, J=3.01, 1.64 Hz), 8.17 (1H, d, J=9.31 Hz), 8.60 (1H, d, J=9.03 Hz), 10.88 (1H, q, J=4.65 Hz); ESIMS found for C₂₁H₂₁F₅N₁₀O m/z 525.2 (M+1).

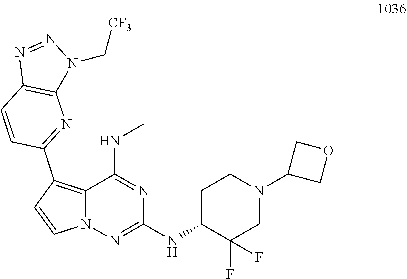 [SMILES: CNc1nc(N[C@@H]2CCN(C3COC3)CC2(F)F)nn2ccc(-c3ccc4nnn(CC(F)(F)F)c4n3)c12]

### (R)—N²-(3,3-Difluoro-1-(oxetan-3-yl)piperidin-4-yl)-N⁴-methyl-5-(3-(2,2,2-trifluoroethyl)-3H-[1,2,3]triazolo[4,5-b]pyridin-5-yl)pyrrolo[2,1-f][1,2,4]triazine-2,4-diamine 1036

Fluffy yellow solid (7 mg, 0.013 mmol, 37.3% yield). ¹H NMR (499 MHz, DMSO-d₆) δ ppm 1.73-1.83 (1H, m), 1.84-1.91 (1H, m), 2.09-2.20 (1H, m), 2.38 (1H, dd, J=27.15, 11.77 Hz), 2.75 (1H, br d, J=11.77 Hz), 2.95-3.06 (1H, m), 3.14 (3H, d, J=4.65 Hz), 3.60 (1H, quin, J=6.30 Hz), 4.21-4.37 (1H, m), 4.44 (2H, dt, J=14.72, 6.19 Hz), 4.55 (2H, td, J=6.57, 3.56 Hz), 5.87 (2H, q, J=9.22 Hz), 6.45 (1H, d, J=9.31 Hz), 7.38 (1H, d, J=3.29 Hz), 7.46 (1H, d, J=3.01 Hz), 8.16 (1H, d, J=9.04 Hz), 8.59 (1H, d, J=9.04 Hz), 10.86 (1H, q, J=4.75 Hz); ESIMS found for C₂₂H₂₃F₅N₁₀O m/z 539.15 (M+1).

### Example 7

Representative compounds were screened using the assay procedure for DYRK1A kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, CA) into 1536-well black-walled round bottom plates (Corning).

The DYRK1A kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant DYRK1A kinase, ATP and Ser/Thr peptide 18 were prepared in 1× Kinase buffer to final concentrations of 0.25 μg/mL, 15 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 uM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio×F100%)−C100%)/((C0%−C100%)+(Em ratio×(F100%−F0%)))]. Dose-response curves were generated, and inhibitory concentration (IC₅₀) values were calculated using non-linear regression curve fit in the Dotmatics' Studies Software (Bishops Stortford, UK).

Table 2 shows the measured activity for representative compounds of Formula I as described herein.

TABLE 2

| Compound | IC₅₀ (μM) | Compound | IC₅₀ (μM) | Compound | IC₅₀ (μM) | Compound | IC₅₀ (μM) |
|---|---|---|---|---|---|---|---|
| 1 | 0.1725 | 221 | 0.0016 | 469 | 0.0010 | 996 | 0.0018 |
| 2 | 0.2702 | 222 | 0.0041 | 472 | 0.0016 | 997 | 0.0017 |
| 20 | 0.0025 | 223 | 0.0011 | 474 | 0.0013 | 998 | 0.0092 |
| 44 | 0.0058 | 224 | 0.0002 | 479 | 0.0006 | 999 | 0.0018 |
| 46 | 0.0061 | 225 | 0.0008 | 480 | 0.0013 | 1000 | 0.0051 |
| 58 | 0.1701 | 226 | 0.0020 | 482 | 0.0031 | 1001 | 0.0022 |
| 114 | 0.7442 | 227 | 0.0033 | 483 | 0.0036 | 1002 | 0.0011 |
| 132 | 0.0017 | 228 | 0.0028 | 484 | 0.0064 | 1003 | 0.0011 |
| 170 | 0.0048 | 339 | 0.0022 | 485 | 0.0073 | 1004 | 0.0011 |
| 171 | 0.0080 | 351 | 0.3648 | 486 | 7.8265 | 1005 | 0.0009 |
| 172 | 0.0009 | 355 | 0.0042 | 487 | 0.0038 | 1006 | 0.0010 |
| 174 | 0.0017 | 363 | 0.0021 | 692 | 0.0009 | 1007 | 0.0041 |
| 182 | 0.0120 | 364 | 0.0006 | 700 | 0.0010 | 1008 | 0.0004 |
| 183 | 0.0041 | 365 | 0.0063 | 701 | 0.0010 | 1009 | 0.0020 |
| 184 | 0.0005 | 366 | 0.0014 | 715 | 0.0004 | 1010 | 0.0005 |
| 185 | 0.0024 | 367 | 0.0064 | 718 | 0.0013 | 1011 | 0.0014 |
| 186 | 0.0015 | 368 | 0.0012 | 729 | 0.0004 | 1012 | 0.0005 |
| 187 | 0.0011 | 369 | 0.0029 | 730 | 0.0021 | 1013 | 0.0009 |
| 188 | 0.0022 | 370 | 0.0155 | 754 | 0.0005 | 1014 | 0.0101 |
| 189 | 0.0013 | 371 | 0.0022 | 762 | 0.0015 | 1015 | 0.0014 |
| 196 | 0.5951 | 372 | 0.0050 | 763 | 0.0014 | 1016 | 0.0015 |
| 197 | 0.0020 | 373 | 0.0038 | 777 | 0.0012 | 1017 | 0.0017 |
| 198 | 0.0006 | 374 | 0.0022 | 780 | 0.0012 | 1018 | 0.0016 |
| 199 | 0.0024 | 375 | 0.0006 | 791 | 0.0011 | 1019 | 0.0017 |
| 200 | 0.0013 | 376 | 0.0015 | 792 | 0.0048 | 1020 | 0.0021 |
| 201 | 0.0007 | 377 | 0.0036 | 816 | 0.0013 | 1021 | 0.0013 |
| 202 | 0.0018 | 378 | 0.0041 | 842 | 0.0015 | 1022 | 0.0004 |
| 203 | 0.0020 | 379 | 0.0034 | 850 | 0.0006 | 1023 | 0.0047 |
| 204 | 0.0008 | 380 | 0.0032 | 853 | 0.0015 | 1024 | 0.0010 |
| 205 | 0.0010 | 381 | 0.0022 | 854 | 0.0021 | 1025 | 0.0101 |
| 210 | 0.0019 | 382 | 0.0033 | 985 | 0.0015 | 1026 | 0.0003 |
| 211 | 0.0037 | 383 | 0.0035 | 986 | 0.0007 | 1027 | 0.0006 |
| 212 | 0.0048 | 384 | 0.0020 | 987 | 0.0008 | 1028 | 0.0013 |
| 213 | 0.0022 | 385 | 0.0046 | 988 | 0.0006 | 1029 | 0.0013 |
| 214 | 0.0011 | 386 | 0.0046 | 989 | 0.0006 | 1030 | 0.1932 |
| 215 | 0.0044 | 387 | 0.0128 | 990 | 0.0006 | 1031 | 0.0040 |
| 216 | 0.0008 | 388 | 0.0047 | 991 | 0.0004 | 1032 | 0.0028 |
| 217 | 0.0017 | 389 | 0.0018 | 992 | 0.0007 | 1033 | 0.0020 |
| 218 | 0.0142 | 441 | 0.0007 | 993 | 0.0006 | 1034 | 0.0019 |
| 219 | 0.0067 | 449 | 0.0005 | 994 | 0.0012 | 1035 | 0.0011 |
| 220 | 0.0097 | 465 | 0.0014 | 995 | 0.0008 | 1036 | 0.0010 |

What is claimed is:

1. A compound, or a pharmaceutically acceptable salt thereof, of Formula I: 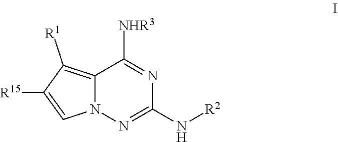
wherein:
R¹ is (9-10 membered heteroaryl) optionally substituted with 1-10 R⁴;
R² is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁵ and —(C₁₋₅ alkylene)ₚcarbocyclyl optionally substituted with 1-12 R⁶, wherein the —(C₁₋₅ alkylene) is optionally substituted with 1-5 halide and/or 1-3 unsubstituted —(C₁₋₃ alkyl);
R³ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R¹⁶;
each R⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl);
each R⁵ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R⁷, -carbocyclyl optionally substituted with 1-12 R¹⁴, —(C₁₋₅ alkylene)ₚOR⁸, and —C(═O)R⁹, wherein the —(C₁₋₅ alkylene) is optionally substituted with 1-5 halide and/or 1-3 unsubstituted —(C₁₋₃ alkyl);
alternatively, two R⁵ attached to the same carbon atom are taken together to form a carbonyl group;
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —(C₁₋₅ alkylene)ₚOR¹⁰, —C(═O)R¹¹, —NHC(═O)R¹², and -carbocyclyl optionally substituted with 1-12 R¹⁴, wherein the —(C₁₋₅ alkylene) is optionally substituted with 1-5 halide and/or 1-3 unsubstituted —(C₁₋₃ alkyl);
each R⁷ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, and -carbocyclyl optionally substituted with 1-12 R⁴;
each R⁸ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁹ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁰ is independently selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(C₁₋₅ alkylene)ₚOR⁸;
each R¹¹ is —N(R¹³)₂;
each R¹² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹³ is independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹⁴ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R¹⁵ is selected from the group consisting of H and halide;
each R¹⁶ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each p is independently 0 or 1; and
wherein each H atom is optionally, independently replaced by ²H (D) (deuterium).

2. The compound of claim 1, wherein R¹⁵ is H.

3. The compound of claim 1, wherein R¹⁵ is F.

4. The compound of claim 2, wherein R¹ is selected from the group consisting of: 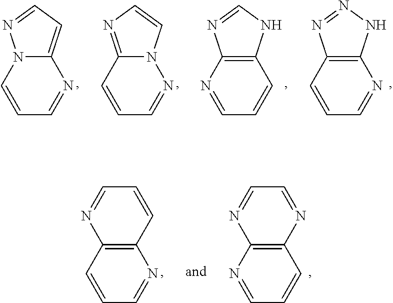
optionally substituted with 1-10 R⁴.

5. The compound of claim 4, wherein R¹ is selected from the group consisting of: 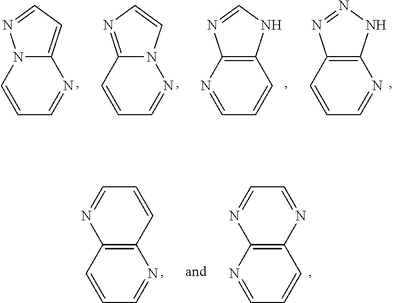
optionally substituted with 1-3 R⁴.

6. The compound of claim 5, wherein R¹ is selected from the group consisting of: 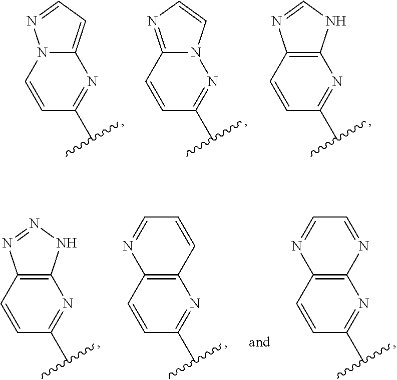 [SMILES: *c1ccn2nccc2n1 ; *c1ccc2nccn2n1 ; *c1ccc2nc[nH]c2n1 ; *c1ccc2nn[nH]c2n1 ; *c1ccc2ncccc2n1 ; *c1ccc2nccnc2n1]
optionally substituted with 1-3 R⁴.

7. The compound of claim 6, wherein R¹ is selected from the group consisting of: 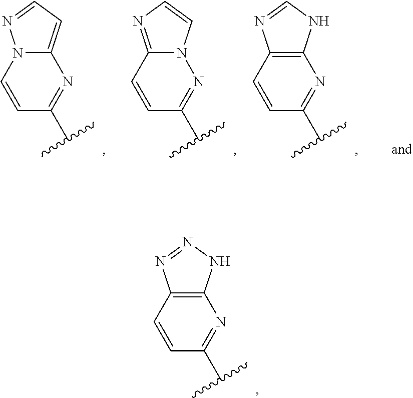 [SMILES: *c1ccn2nccc2n1 ; *c1ccc2nccn2n1 ; *c1ccc2nc[nH]c2n1 ; *c1ccc2nn[nH]c2n1]
optionally substituted with 1-3 R⁴.

8. The compound of claim 7, wherein R⁴ is selected from the group consisting of halide, unsubstituted —(C₁₋₄ alkyl), and unsubstituted —(C₁₋₄ haloalkyl).

9. The compound of claim 8, wherein R¹ is selected from the group consisting of:

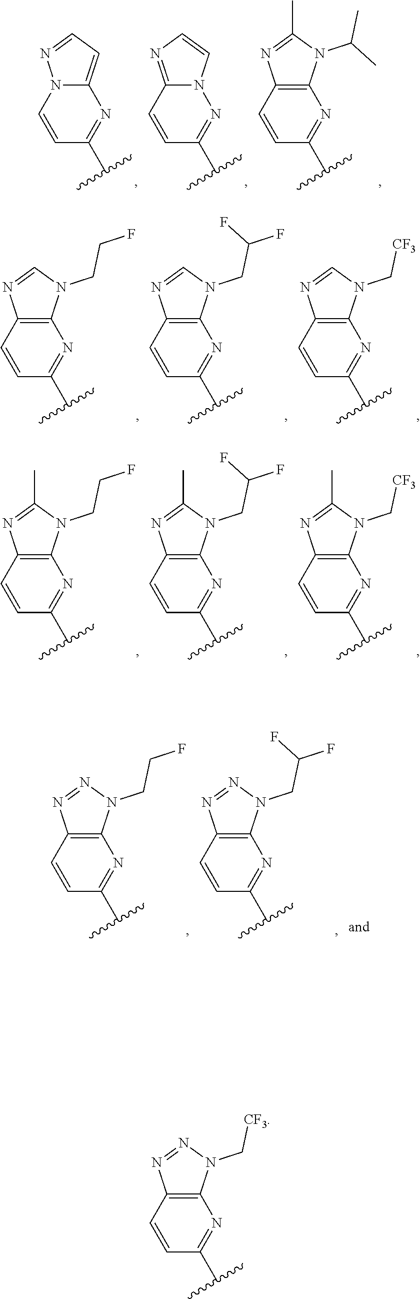

10. The compound of claim 9, wherein R¹ is selected from the group consisting of:

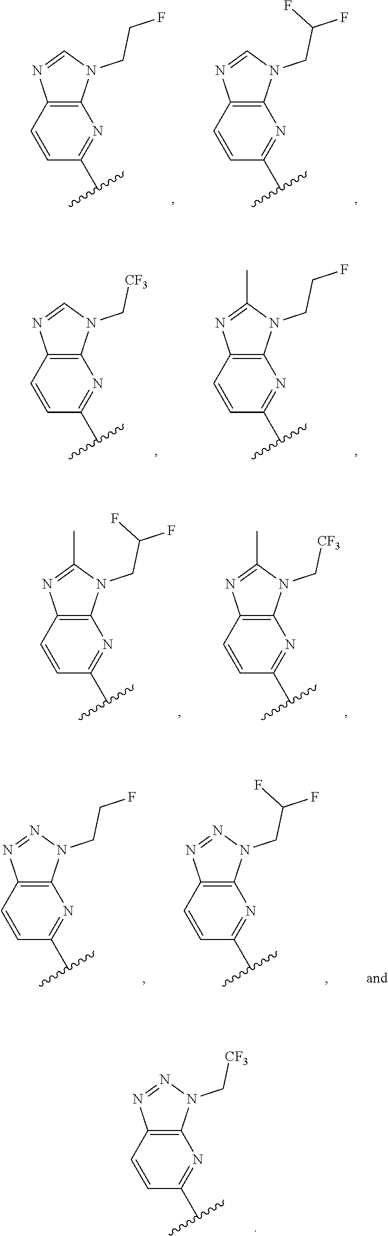

11. The compound of claim 1, wherein R² is selected from the group consisting of -heterocyclyl optionally substituted with 1-4 R⁵, and -carbocyclyl optionally substituted with 1-4 R⁶.

12. The compound of claim 10, wherein R² is selected from the group consisting of -heterocyclyl optionally substituted with 1-4 R⁵, and -carbocyclyl optionally substituted with 1-4 R⁶.

13. The compound of claim 1, wherein R² is selected from the group consisting of: 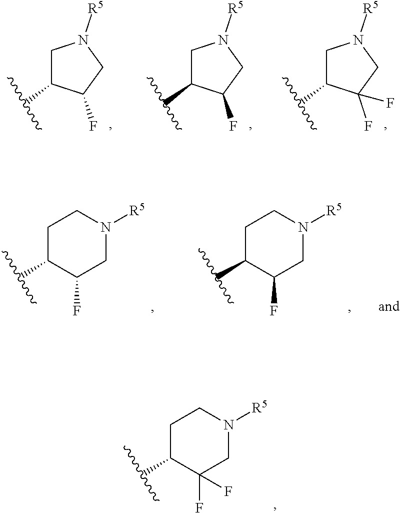
wherein each R⁵ is selected from the group consisting of Me, —C(═O)Me, and

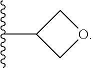 [SMILES: *C1COC1]

14. The compound of claim 10, wherein R² is selected from the group consisting of: 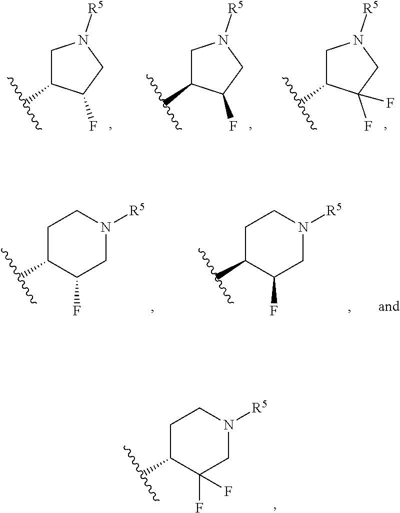
wherein each R⁵ is selected from the group consisting of Me, —C(═O)Me, and

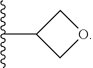 [SMILES: *C1COC1]

15. The compound of claim 1, wherein R³ is selected from the group consisting of H and unsubstituted —(C₁₋₃ alkyl).

16. The compound of claim 1, wherein R³ is H.

17. The compound of claim 14, wherein R³ is H.

18. The compound of claim 1, wherein R³ is methyl.

19. The compound of claim 14, wherein R³ is methyl.

20. The compound of claim 1, wherein the compound of Formula I is selected from the group consisting of 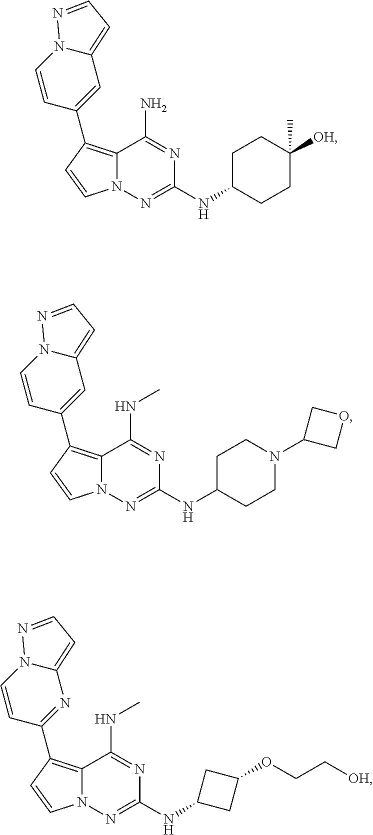 [SMILES: C[C@]1(O)CC[C@H](Nc2nc(N)c3c(-c4ccn5nccc5c4)ccn3n2)CC1 ; CNc1nc(NC2CCN(C3COC3)CC2)nn2ccc(-c3ccn4nccc4c3)c12 ; CNc1nc(N[C@H]2C[C@@H](OCCO)C2)nn2ccc(-c3ccn4nccc4n3)c12] 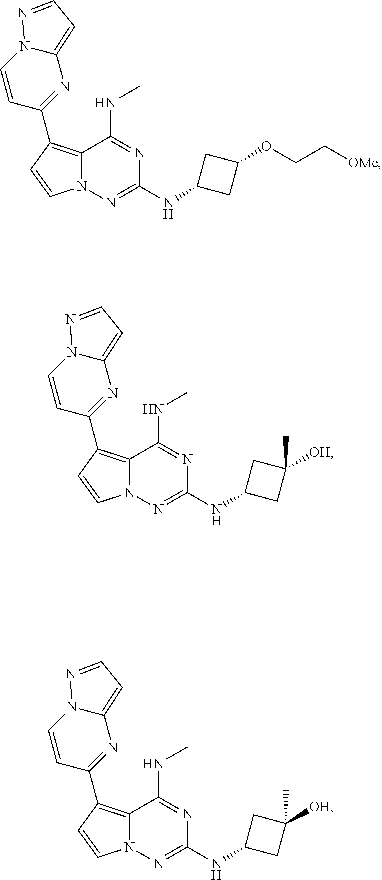 [SMILES: CNc1nc(N[C@H]2C[C@@H](OCCOC)C2)nn2ccc(-c3ccn4nccc4n3)c12 ; CNc1nc(N[C@H]2C[C@@](C)(O)C2)nn2ccc(-c3ccn4nccc4n3)c12 ; CNc1nc(N[C@H]2C[C@](C)(O)C2)nn2ccc(-c3ccn4nccc4n3)c12] 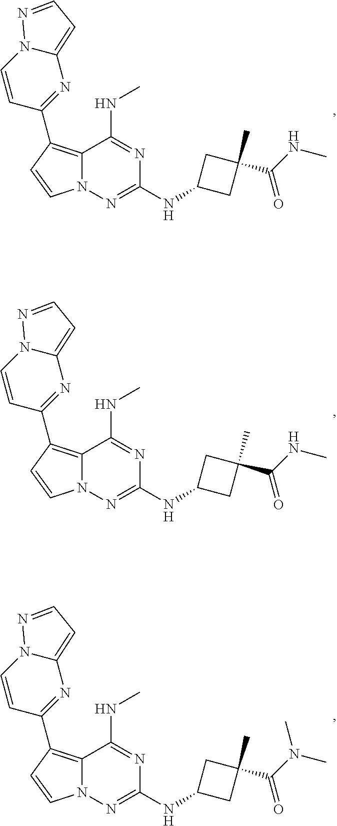 [SMILES: CNc1nc(N[C@H]2C[C@@](C)(C(=O)NC)C2)nn2ccc(-c3ccn4nccc4n3)c12 ; CNc1nc(N[C@H]2C[C@](C)(C(=O)NC)C2)nn2ccc(-c3ccn4nccc4n3)c12 ; CNc1nc(N[C@H]2C[C@@](C)(C(=O)N(C)C)C2)nn2ccc(-c3ccn4nccc4n3)c12] 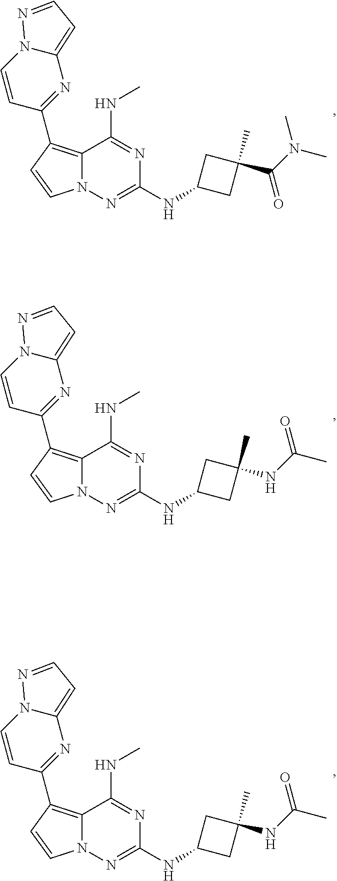 [SMILES: CNc1nc(N[C@H]2C[C@](C)(C(=O)N(C)C)C2)nn2ccc(-c3ccn4nccc4n3)c12 ; CNc1nc(N[C@H]2C[C@@](C)(NC(C)=O)C2)nn2ccc(-c3ccn4nccc4n3)c12 ; CNc1nc(N[C@H]2C[C@](C)(NC(C)=O)C2)nn2ccc(-c3ccn4nccc4n3)c12] 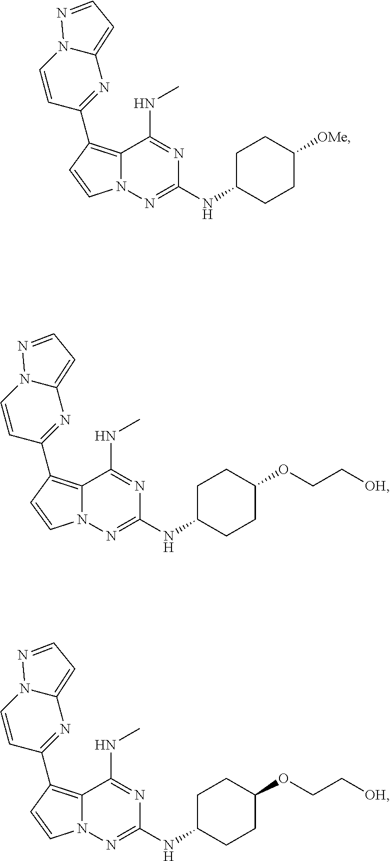 [SMILES: CNc1nc(N[C@H]2CC[C@@H](OC)CC2)nn2ccc(-c3ccn4nccc4n3)c12 ; CNc1nc(N[C@H]2CC[C@@H](OCCO)CC2)nn2ccc(-c3ccn4nccc4n3)c12 ; CNc1nc(N[C@H]2CC[C@H](OCCO)CC2)nn2ccc(-c3ccn4nccc4n3)c12] 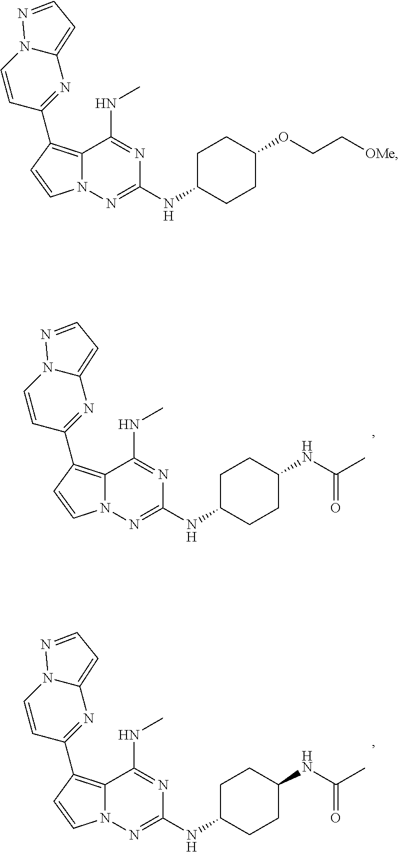 [SMILES: CNc1nc(N[C@H]2CC[C@@H](OCCOC)CC2)nn2ccc(-c3ccn4nccc4n3)c12 ; CNc1nc(N[C@H]2CC[C@@H](NC(C)=O)CC2)nn2ccc(-c3ccn4nccc4n3)c12 ; CNc1nc(N[C@H]2CC[C@H](NC(C)=O)CC2)nn2ccc(-c3ccn4nccc4n3)c12] 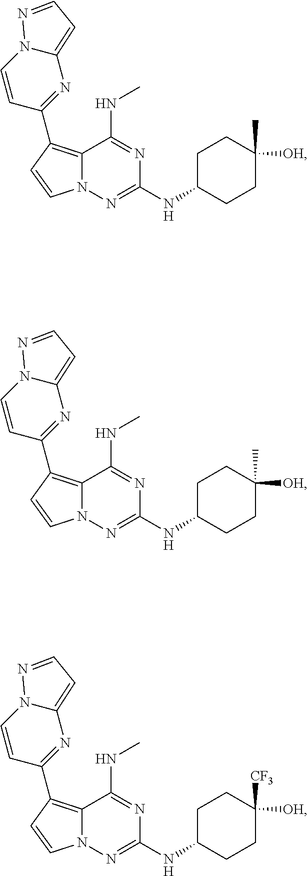 [SMILES: CNc1nc(N[C@H]2CC[C@@](C)(O)CC2)nn2ccc(-c3ccn4nccc4n3)c12 ; CNc1nc(N[C@H]2CC[C@](C)(O)CC2)nn2ccc(-c3ccn4nccc4n3)c12 ; CNc1nc(N[C@H]2CC[C@](O)(C(F)(F)F)CC2)nn2ccc(-c3ccn4nccc4n3)c12] 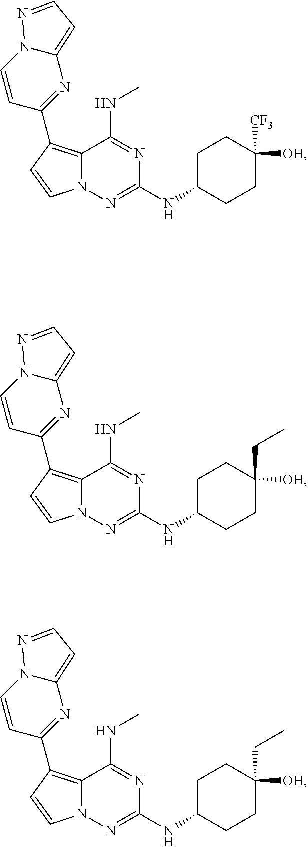 [SMILES: CNc1nc(N[C@H]2CC[C@@](O)(C(F)(F)F)CC2)nn2ccc(-c3ccn4nccc4n3)c12 ; CC[C@]1(O)CC[C@@H](Nc2nc(NC)c3c(-c4ccn5nccc5n4)ccn3n2)CC1 ; CC[C@]1(O)CC[C@H](Nc2nc(NC)c3c(-c4ccn5nccc5n4)ccn3n2)CC1] 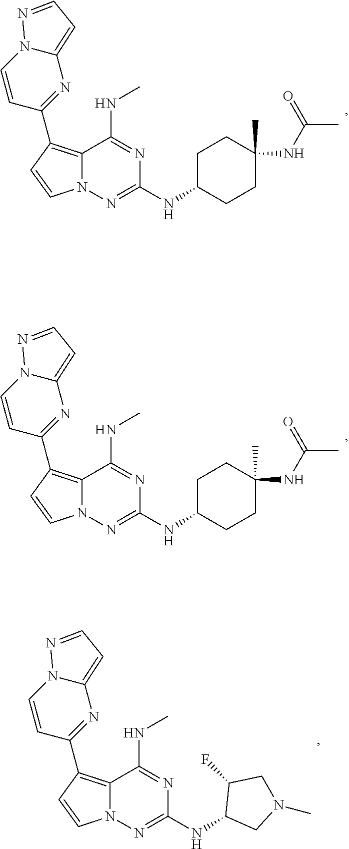 [SMILES: CNc1nc(N[C@H]2CC[C@@](C)(NC(C)=O)CC2)nn2ccc(-c3ccn4nccc4n3)c12 ; CNc1nc(N[C@H]2CC[C@](C)(NC(C)=O)CC2)nn2ccc(-c3ccn4nccc4n3)c12 ; CNc1nc(N[C@H]2CN(C)C[C@H]2F)nn2ccc(-c3ccn4nccc4n3)c12] 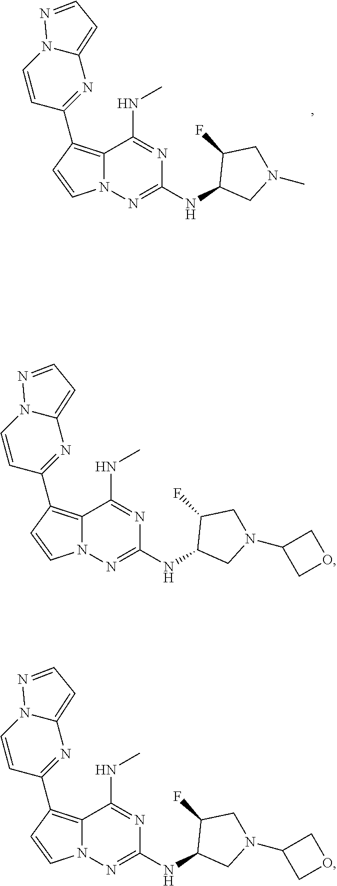 [SMILES: CNc1nc(N[C@@H]2CN(C)C[C@@H]2F)nn2ccc(-c3ccn4nccc4n3)c12 ; CNc1nc(N[C@H]2CN(C3COC3)C[C@H]2F)nn2ccc(-c3ccn4nccc4n3)c12 ; CNc1nc(N[C@@H]2CN(C3COC3)C[C@@H]2F)nn2ccc(-c3ccn4nccc4n3)c12] 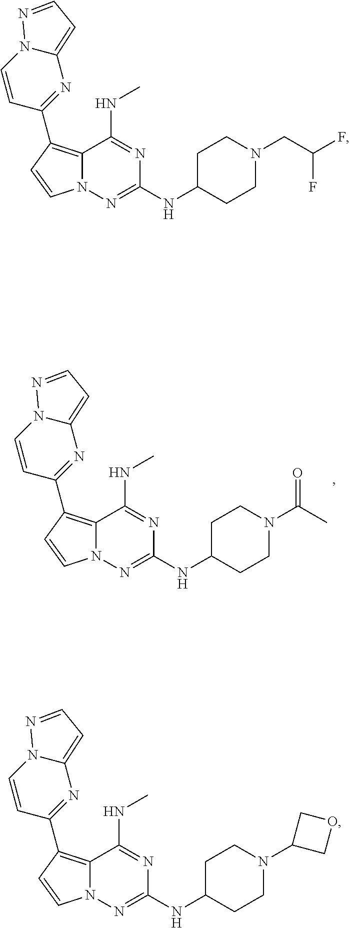 [SMILES: CNc1nc(NC2CCN(CC(F)F)CC2)nn2ccc(-c3ccn4nccc4n3)c12 ; CNc1nc(NC2CCN(C(C)=O)CC2)nn2ccc(-c3ccn4nccc4n3)c12 ; CNc1nc(NC2CCN(C3COC3)CC2)nn2ccc(-c3ccn4nccc4n3)c12] 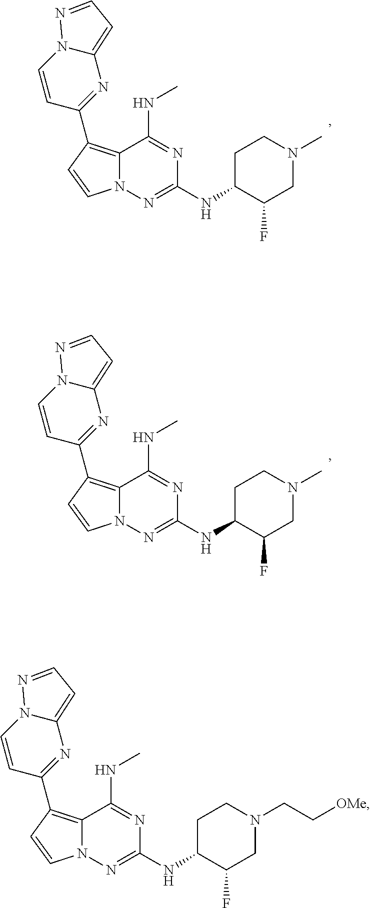 [SMILES: CNc1nc(N[C@@H]2CCN(C)C[C@@H]2F)nn2ccc(-c3ccn4nccc4n3)c12 ; CNc1nc(N[C@H]2CCN(C)C[C@H]2F)nn2ccc(-c3ccn4nccc4n3)c12 ; CNc1nc(N[C@@H]2CCN(CCOC)C[C@@H]2F)nn2ccc(-c3ccn4nccc4n3)c12] 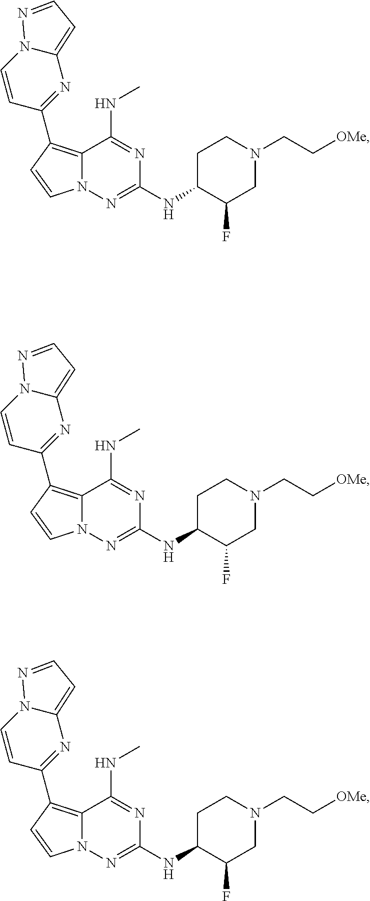 [SMILES: CNc1nc(N[C@@H]2CCN(CCOC)C[C@H]2F)nn2ccc(-c3ccn4nccc4n3)c12 ; CNc1nc(N[C@H]2CCN(CCOC)C[C@@H]2F)nn2ccc(-c3ccn4nccc4n3)c12 ; CNc1nc(N[C@H]2CCN(CCOC)C[C@H]2F)nn2ccc(-c3ccn4nccc4n3)c12] 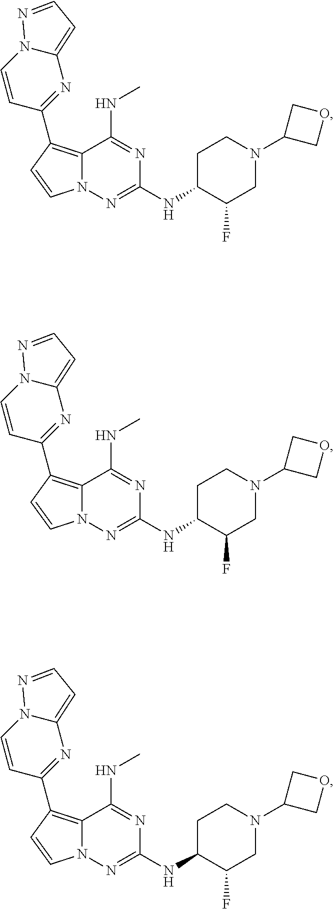 [SMILES: CNc1nc(N[C@@H]2CCN(C3COC3)C[C@@H]2F)nn2ccc(-c3ccn4nccc4n3)c12 ; CNc1nc(N[C@@H]2CCN(C3COC3)C[C@H]2F)nn2ccc(-c3ccn4nccc4n3)c12 ; CNc1nc(N[C@H]2CCN(C3COC3)C[C@@H]2F)nn2ccc(-c3ccn4nccc4n3)c12] 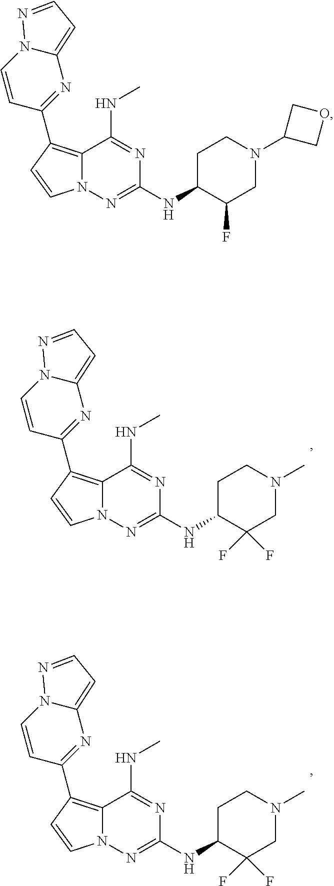 [SMILES: CNc1nc(N[C@H]2CCN(C3COC3)C[C@H]2F)nn2ccc(-c3ccn4nccc4n3)c12 ; CNc1nc(N[C@@H]2CCN(C)CC2(F)F)nn2ccc(-c3ccn4nccc4n3)c12 ; CNc1nc(N[C@H]2CCN(C)CC2(F)F)nn2ccc(-c3ccn4nccc4n3)c12] 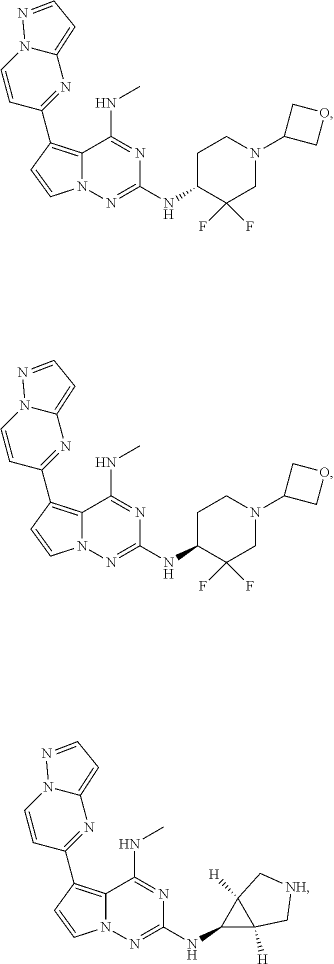 [SMILES: CNc1nc(N[C@@H]2CCN(C3COC3)CC2(F)F)nn2ccc(-c3ccn4nccc4n3)c12 ; CNc1nc(N[C@H]2CCN(C3COC3)CC2(F)F)nn2ccc(-c3ccn4nccc4n3)c12 ; CNc1nc(N[C@@H]2[C@@H]3CNC[C@@H]32)nn2ccc(-c3ccn4nccc4n3)c12] 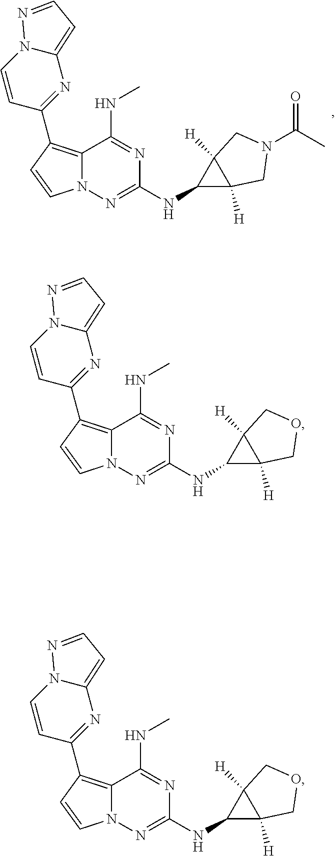 [SMILES: CNc1nc(N[C@@H]2[C@@H]3CN(C(C)=O)C[C@@H]32)nn2ccc(-c3ccn4nccc4n3)c12 ; CNc1nc(N[C@H]2[C@@H]3COC[C@@H]32)nn2ccc(-c3ccn4nccc4n3)c12 ; CNc1nc(N[C@@H]2[C@@H]3COC[C@@H]32)nn2ccc(-c3ccn4nccc4n3)c12] 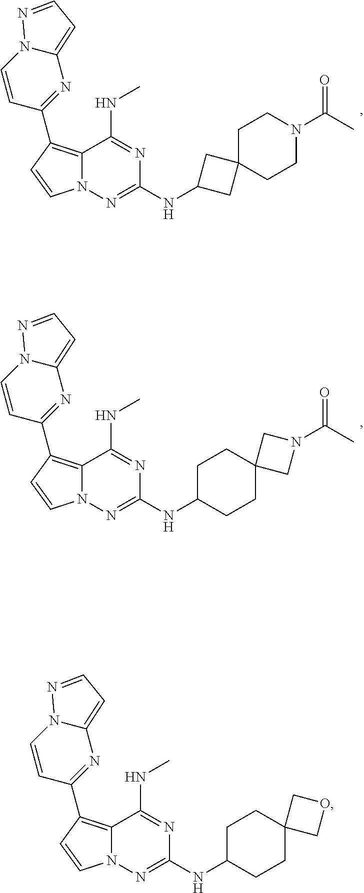 [SMILES: CNc1nc(NC2CC3(CCN(C(C)=O)CC3)C2)nn2ccc(-c3ccn4nccc4n3)c12 ; CNc1nc(NC2CCC3(CC2)CN(C(C)=O)C3)nn2ccc(-c3ccn4nccc4n3)c12 ; CNc1nc(NC2CCC3(CC2)COC3)nn2ccc(-c3ccn4nccc4n3)c12] 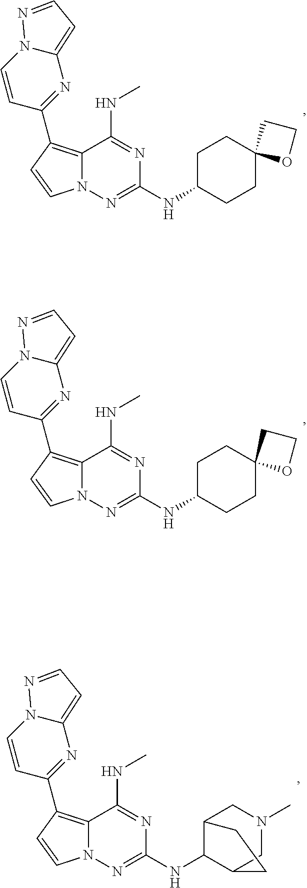 [SMILES: CNc1nc(N[C@H]2CC[C@@]3(CCO3)CC2)nn2ccc(-c3ccn4nccc4n3)c12 ; CNc1nc(N[C@H]2CC[C@]3(CCO3)CC2)nn2ccc(-c3ccn4nccc4n3)c12 ; CNc1nc(NC2C3CCC2CN(C)C3)nn2ccc(-c3ccn4nccc4n3)c12] 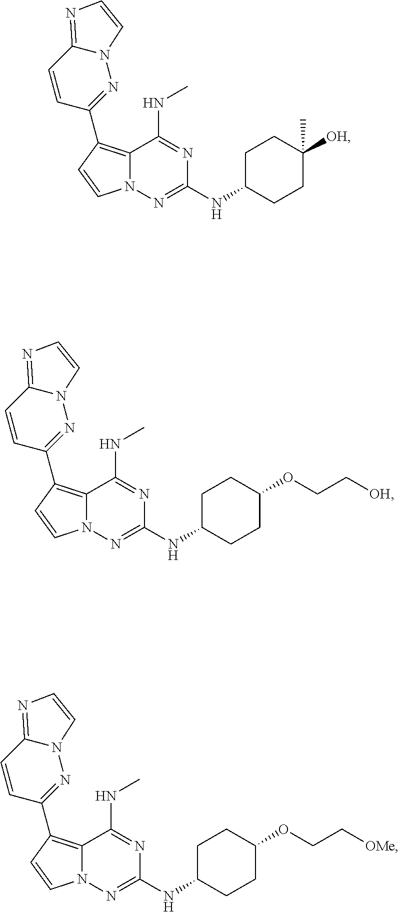 [SMILES: CNc1nc(N[C@H]2CC[C@](C)(O)CC2)nn2ccc(-c3ccc4nccn4n3)c12 ; CNc1nc(N[C@H]2CC[C@@H](OCCO)CC2)nn2ccc(-c3ccc4nccn4n3)c12 ; CNc1nc(N[C@H]2CC[C@@H](OCCOC)CC2)nn2ccc(-c3ccc4nccn4n3)c12] 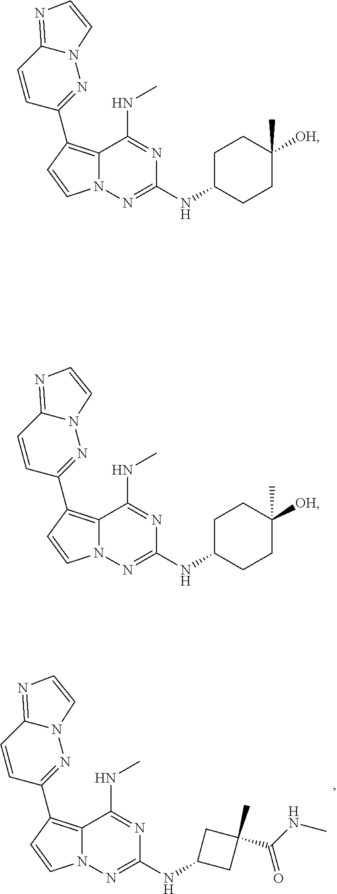 [SMILES: CNc1nc(N[C@H]2CC[C@@](C)(O)CC2)nn2ccc(-c3ccc4nccn4n3)c12 ; CNc1nc(N[C@H]2CC[C@](C)(O)CC2)nn2ccc(-c3ccc4nccn4n3)c12 ; CNc1nc(N[C@H]2C[C@@](C)(C(=O)NC)C2)nn2ccc(-c3ccc4nccn4n3)c12] 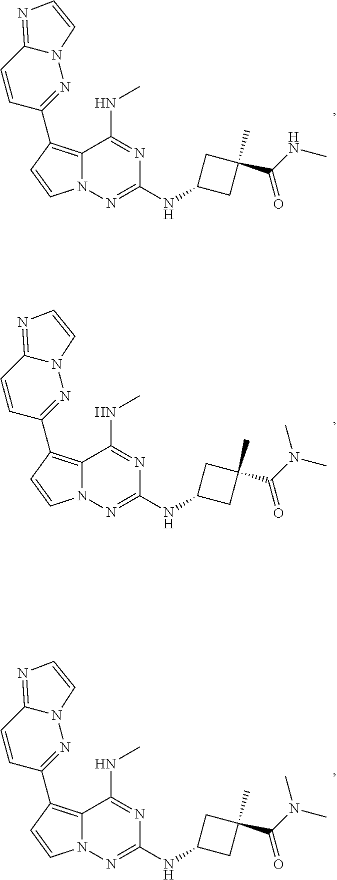 [SMILES: CNc1nc(N[C@H]2C[C@](C)(C(=O)NC)C2)nn2ccc(-c3ccc4nccn4n3)c12 ; CNc1nc(N[C@H]2C[C@@](C)(C(=O)N(C)C)C2)nn2ccc(-c3ccc4nccn4n3)c12 ; CNc1nc(N[C@H]2C[C@](C)(C(=O)N(C)C)C2)nn2ccc(-c3ccc4nccn4n3)c12] 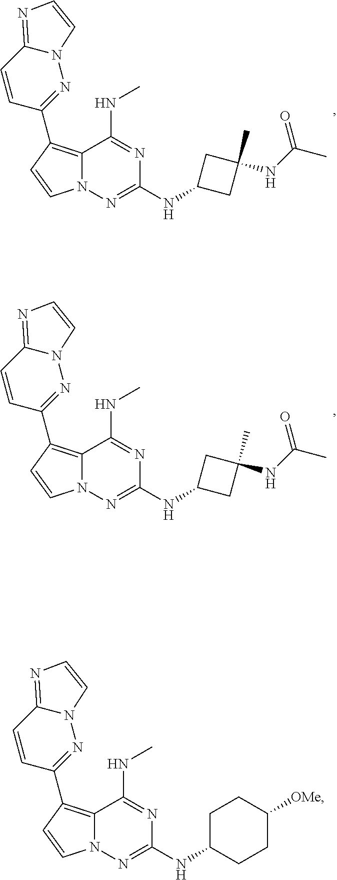 [SMILES: CNc1nc(N[C@H]2C[C@@](C)(NC(C)=O)C2)nn2ccc(-c3ccc4nccn4n3)c12 ; CNc1nc(N[C@H]2C[C@](C)(NC(C)=O)C2)nn2ccc(-c3ccc4nccn4n3)c12 ; CNc1nc(N[C@H]2CC[C@@H](OC)CC2)nn2ccc(-c3ccc4nccn4n3)c12] 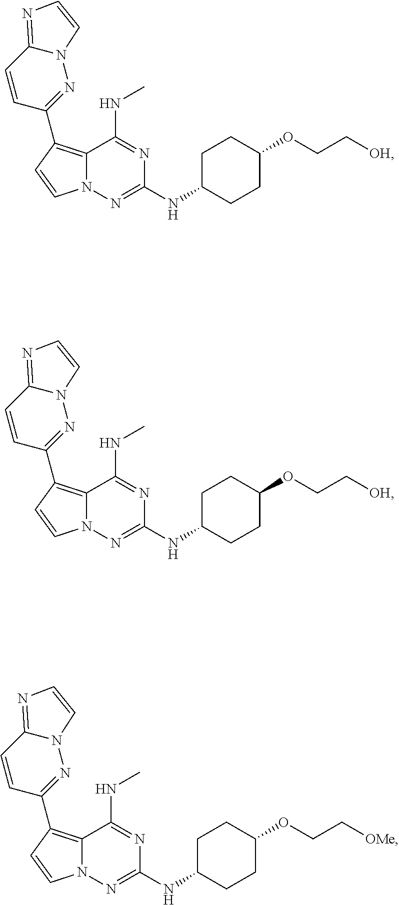 [SMILES: CNc1nc(N[C@H]2CC[C@@H](OCCO)CC2)nn2ccc(-c3ccc4nccn4n3)c12 ; CNc1nc(N[C@H]2CC[C@H](OCCO)CC2)nn2ccc(-c3ccc4nccn4n3)c12 ; CNc1nc(N[C@H]2CC[C@@H](OCCOC)CC2)nn2ccc(-c3ccc4nccn4n3)c12] 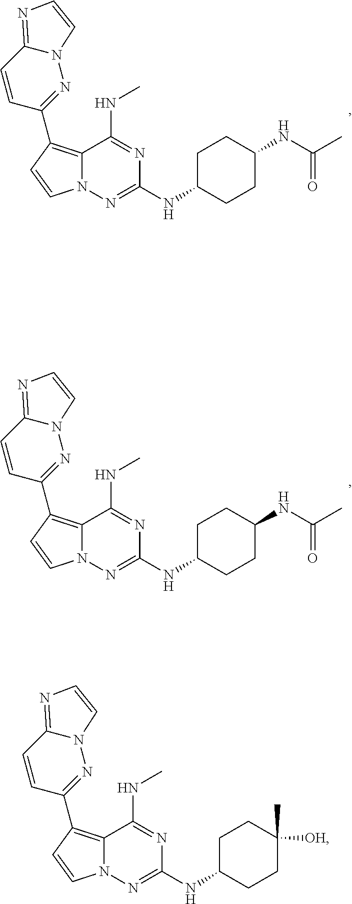 [SMILES: CNc1nc(N[C@H]2CC[C@@H](NC(C)=O)CC2)nn2ccc(-c3ccc4nccn4n3)c12 ; CNc1nc(N[C@H]2CC[C@H](NC(C)=O)CC2)nn2ccc(-c3ccc4nccn4n3)c12 ; CNc1nc(N[C@H]2CC[C@@](C)(O)CC2)nn2ccc(-c3ccc4nccn4n3)c12] 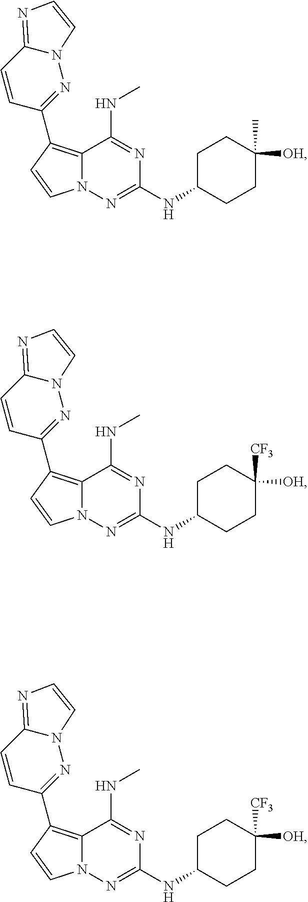 [SMILES: CNc1nc(N[C@H]2CC[C@](C)(O)CC2)nn2ccc(-c3ccc4nccn4n3)c12 ; CNc1nc(N[C@H]2CC[C@](O)(C(F)(F)F)CC2)nn2ccc(-c3ccc4nccn4n3)c12 ; CNc1nc(N[C@H]2CC[C@@](O)(C(F)(F)F)CC2)nn2ccc(-c3ccc4nccn4n3)c12] 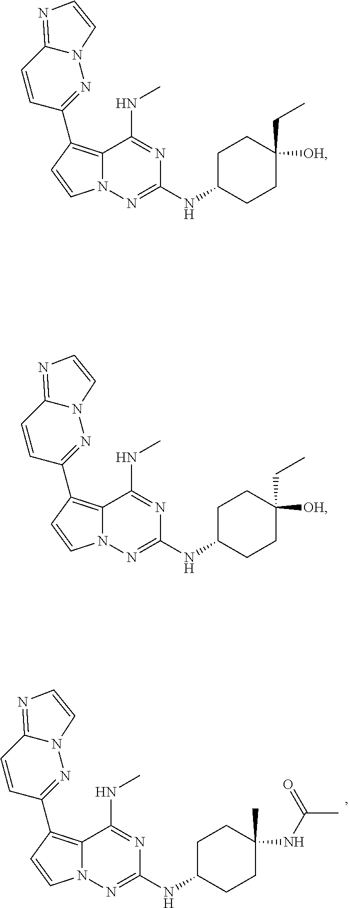 [SMILES: CC[C@]1(O)CC[C@@H](Nc2nc(NC)c3c(-c4ccc5nccn5n4)ccn3n2)CC1 ; CC[C@]1(O)CC[C@H](Nc2nc(NC)c3c(-c4ccc5nccn5n4)ccn3n2)CC1 ; CNc1nc(N[C@H]2CC[C@@](C)(NC(C)=O)CC2)nn2ccc(-c3ccc4nccn4n3)c12] 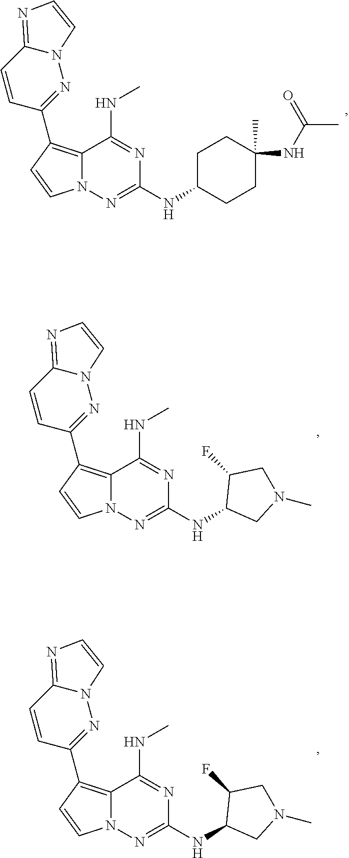 [SMILES: CNc1nc(N[C@H]2CC[C@](C)(NC(C)=O)CC2)nn2ccc(-c3ccc4nccn4n3)c12 ; CNc1nc(N[C@H]2CN(C)C[C@H]2F)nn2ccc(-c3ccc4nccn4n3)c12 ; CNc1nc(N[C@@H]2CN(C)C[C@@H]2F)nn2ccc(-c3ccc4nccn4n3)c12] 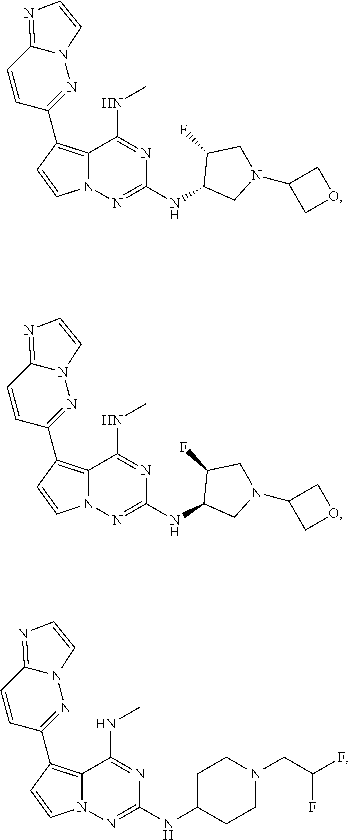 [SMILES: CNc1nc(N[C@H]2CN(C3COC3)C[C@H]2F)nn2ccc(-c3ccc4nccn4n3)c12 ; CNc1nc(N[C@@H]2CN(C3COC3)C[C@@H]2F)nn2ccc(-c3ccc4nccn4n3)c12 ; CNc1nc(NC2CCN(CC(F)F)CC2)nn2ccc(-c3ccc4nccn4n3)c12] 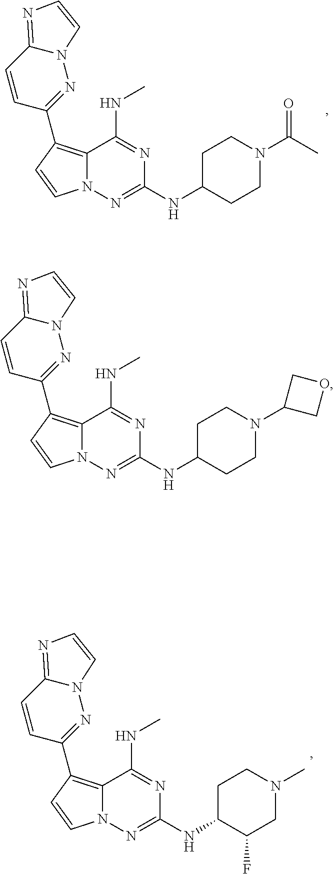 [SMILES: CNc1nc(NC2CCN(C(C)=O)CC2)nn2ccc(-c3ccc4nccn4n3)c12 ; CNc1nc(NC2CCN(C3COC3)CC2)nn2ccc(-c3ccc4nccn4n3)c12 ; CNc1nc(N[C@@H]2CCN(C)C[C@@H]2F)nn2ccc(-c3ccc4nccn4n3)c12] 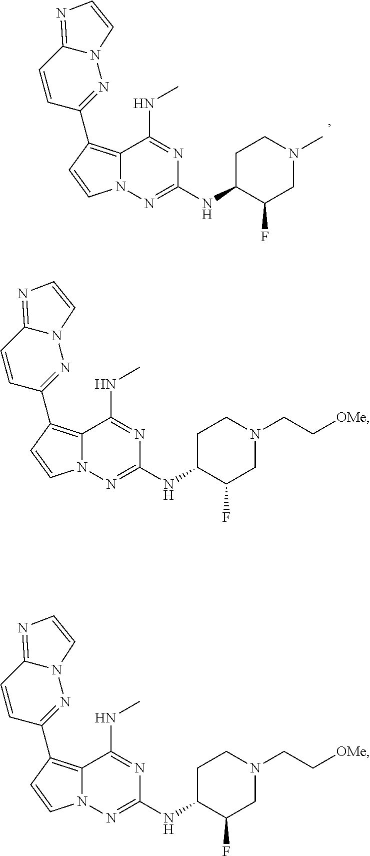 [SMILES: CNc1nc(N[C@H]2CCN(C)C[C@H]2F)nn2ccc(-c3ccc4nccn4n3)c12 ; CNc1nc(N[C@@H]2CCN(CCOC)C[C@@H]2F)nn2ccc(-c3ccc4nccn4n3)c12 ; CNc1nc(N[C@@H]2CCN(CCOC)C[C@H]2F)nn2ccc(-c3ccc4nccn4n3)c12] 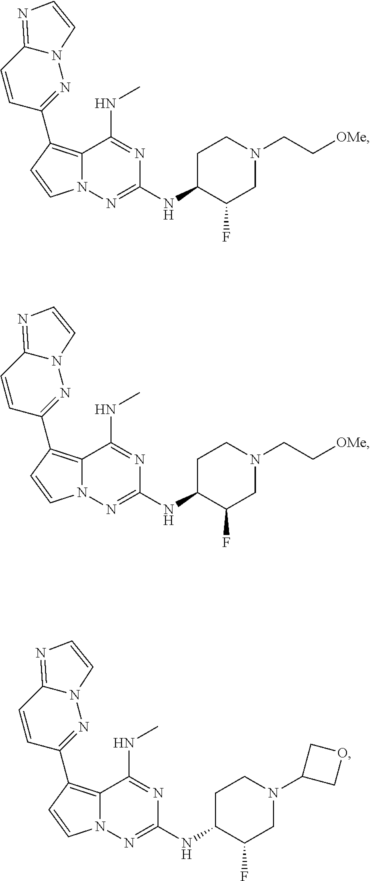 [SMILES: CNc1nc(N[C@H]2CCN(CCOC)C[C@@H]2F)nn2ccc(-c3ccc4nccn4n3)c12 ; CNc1nc(N[C@H]2CCN(CCOC)C[C@H]2F)nn2ccc(-c3ccc4nccn4n3)c12 ; CNc1nc(N[C@@H]2CCN(C3COC3)C[C@@H]2F)nn2ccc(-c3ccc4nccn4n3)c12] 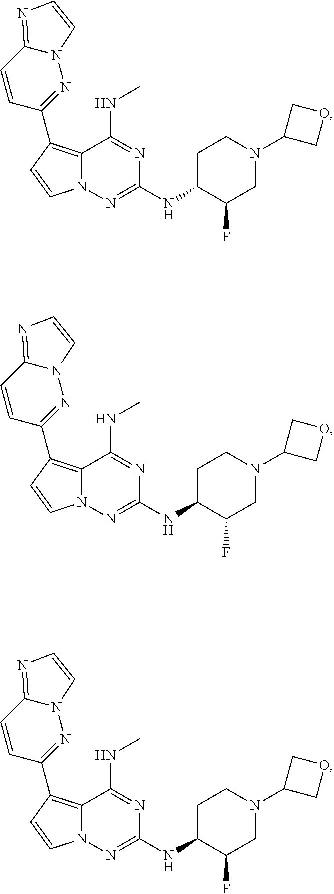 [SMILES: CNc1nc(N[C@@H]2CCN(C3COC3)C[C@H]2F)nn2ccc(-c3ccc4nccn4n3)c12 ; CNc1nc(N[C@H]2CCN(C3COC3)C[C@@H]2F)nn2ccc(-c3ccc4nccn4n3)c12 ; CNc1nc(N[C@H]2CCN(C3COC3)C[C@H]2F)nn2ccc(-c3ccc4nccn4n3)c12] 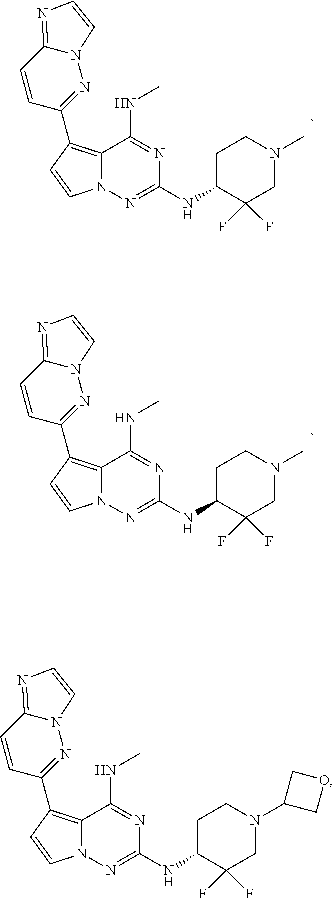 [SMILES: CNc1nc(N[C@@H]2CCN(C)CC2(F)F)nn2ccc(-c3ccc4nccn4n3)c12 ; CNc1nc(N[C@H]2CCN(C)CC2(F)F)nn2ccc(-c3ccc4nccn4n3)c12 ; CNc1nc(N[C@@H]2CCN(C3COC3)CC2(F)F)nn2ccc(-c3ccc4nccn4n3)c12] 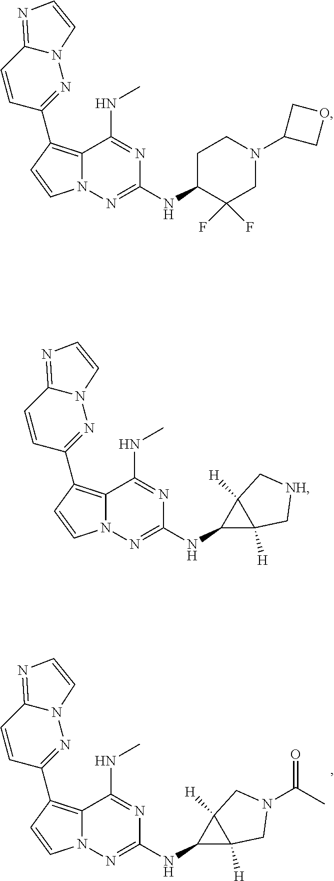 [SMILES: CNc1nc(N[C@H]2CCN(C3COC3)CC2(F)F)nn2ccc(-c3ccc4nccn4n3)c12 ; CNc1nc(N[C@@H]2[C@@H]3CNC[C@@H]32)nn2ccc(-c3ccc4nccn4n3)c12 ; CNc1nc(N[C@@H]2[C@@H]3CN(C(C)=O)C[C@@H]32)nn2ccc(-c3ccc4nccn4n3)c12] 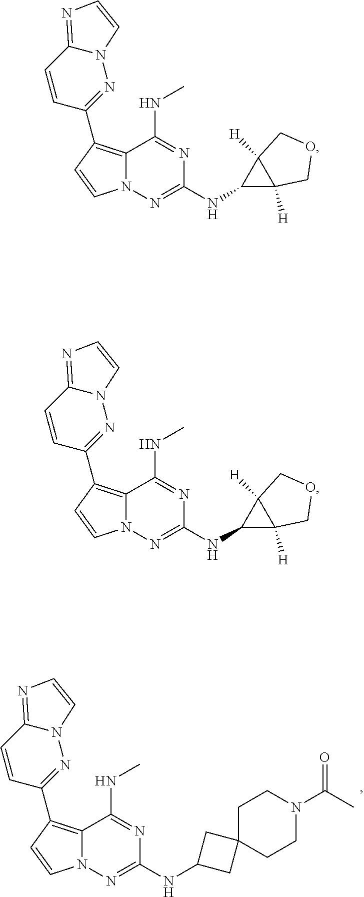 [SMILES: CNc1nc(N[C@H]2[C@@H]3COC[C@@H]32)nn2ccc(-c3ccc4nccn4n3)c12 ; CNc1nc(N[C@@H]2[C@@H]3COC[C@@H]32)nn2ccc(-c3ccc4nccn4n3)c12 ; CNc1nc(NC2CC3(CCN(C(C)=O)CC3)C2)nn2ccc(-c3ccc4nccn4n3)c12] 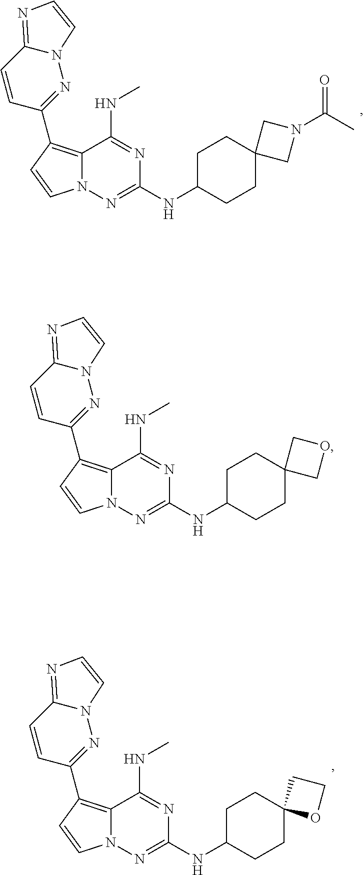 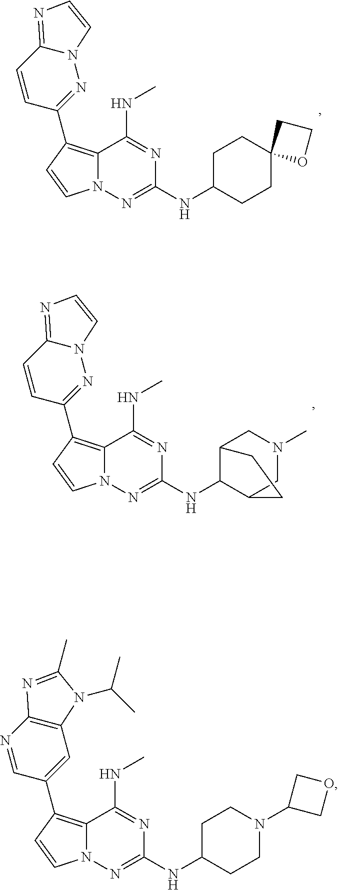 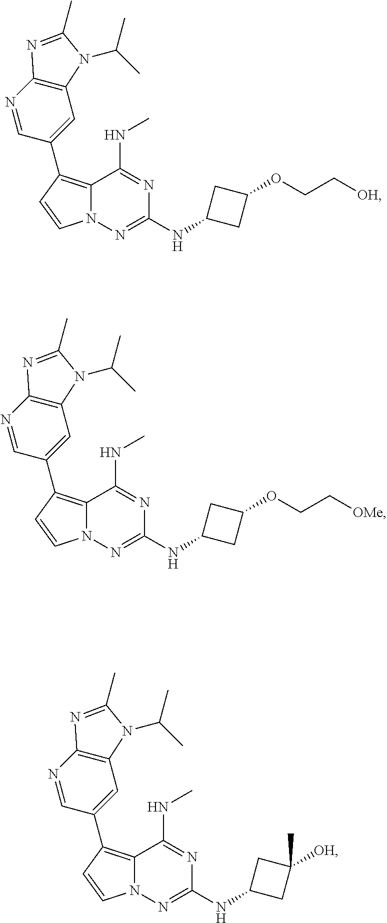 [SMILES: CNc1nc(N[C@H]2C[C@@H](OCCO)C2)nn2ccc(-c3cnc4nc(C)n(C(C)C)c4c3)c12 ; CNc1nc(N[C@H]2C[C@@H](OCCOC)C2)nn2ccc(-c3cnc4nc(C)n(C(C)C)c4c3)c12 ; CNc1nc(N[C@H]2C[C@@](C)(O)C2)nn2ccc(-c3cnc4nc(C)n(C(C)C)c4c3)c12] 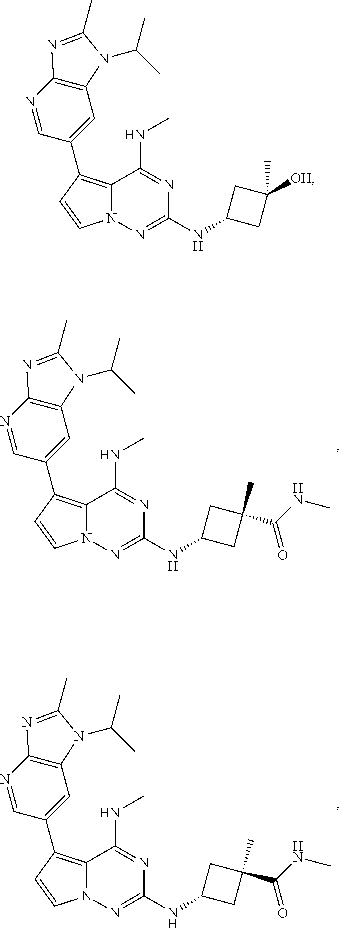 [SMILES: CNc1nc(N[C@H]2C[C@](C)(O)C2)nn2ccc(-c3cnc4nc(C)n(C(C)C)c4c3)c12 ; CNc1nc(N[C@H]2C[C@@](C)(C(=O)NC)C2)nn2ccc(-c3cnc4nc(C)n(C(C)C)c4c3)c12 ; CNc1nc(N[C@H]2C[C@](C)(C(=O)NC)C2)nn2ccc(-c3cnc4nc(C)n(C(C)C)c4c3)c12] 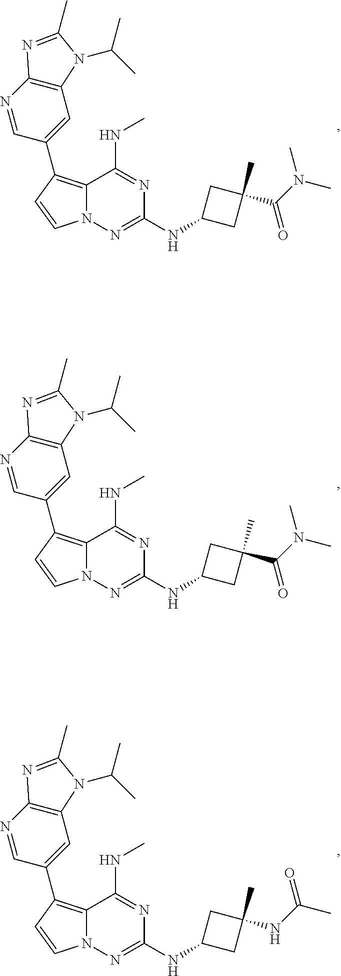 [SMILES: CNc1nc(N[C@H]2C[C@@](C)(C(=O)N(C)C)C2)nn2ccc(-c3cnc4nc(C)n(C(C)C)c4c3)c12 ; CNc1nc(N[C@H]2C[C@](C)(C(=O)N(C)C)C2)nn2ccc(-c3cnc4nc(C)n(C(C)C)c4c3)c12 ; CNc1nc(N[C@H]2C[C@@](C)(NC(C)=O)C2)nn2ccc(-c3cnc4nc(C)n(C(C)C)c4c3)c12] 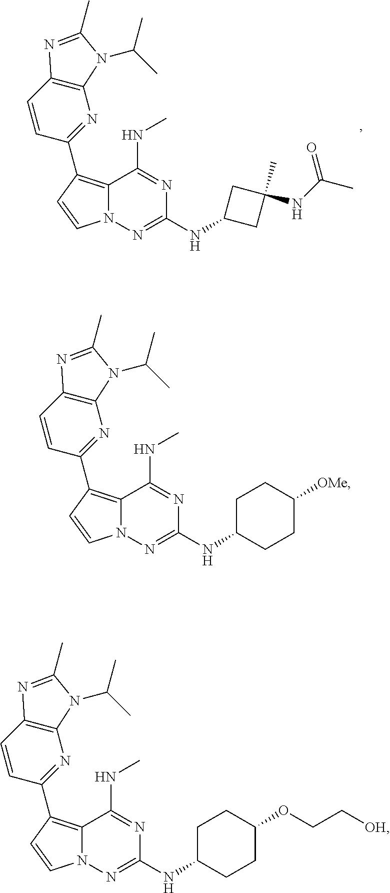 [SMILES: CNc1nc(N[C@H]2C[C@](C)(NC(C)=O)C2)nn2ccc(-c3ccc4nc(C)n(C(C)C)c4n3)c12 ; CNc1nc(N[C@H]2CC[C@@H](OC)CC2)nn2ccc(-c3ccc4nc(C)n(C(C)C)c4n3)c12 ; CNc1nc(N[C@H]2CC[C@@H](OCCO)CC2)nn2ccc(-c3ccc4nc(C)n(C(C)C)c4n3)c12] 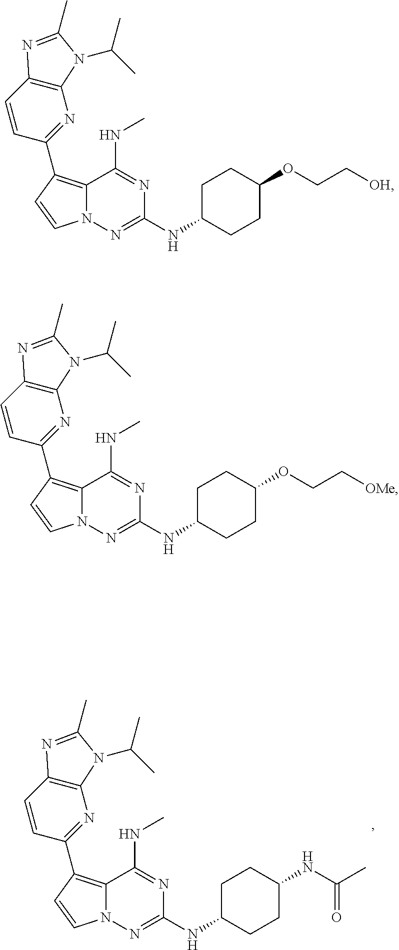 [SMILES: CNc1nc(N[C@H]2CC[C@H](OCCO)CC2)nn2ccc(-c3ccc4nc(C)n(C(C)C)c4n3)c12 ; CNc1nc(N[C@H]2CC[C@@H](OCCOC)CC2)nn2ccc(-c3ccc4nc(C)n(C(C)C)c4n3)c12 ; CNc1nc(N[C@H]2CC[C@@H](NC(C)=O)CC2)nn2ccc(-c3ccc4nc(C)n(C(C)C)c4n3)c12] 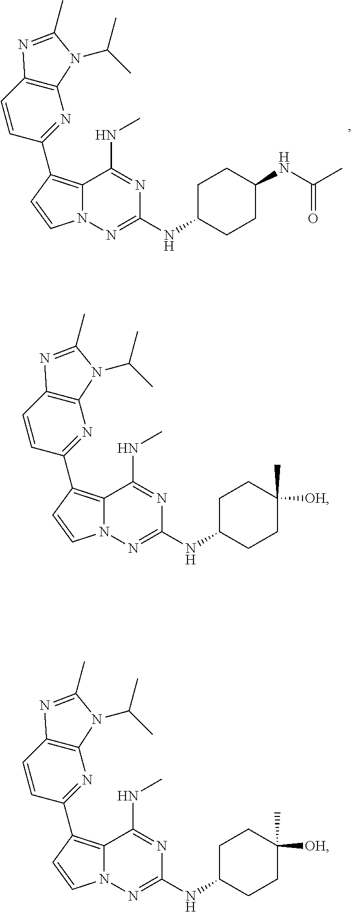 [SMILES: CNc1nc(N[C@H]2CC[C@H](NC(C)=O)CC2)nn2ccc(-c3ccc4nc(C)n(C(C)C)c4n3)c12 ; CNc1nc(N[C@H]2CC[C@@](C)(O)CC2)nn2ccc(-c3ccc4nc(C)n(C(C)C)c4n3)c12 ; CNc1nc(N[C@H]2CC[C@](C)(O)CC2)nn2ccc(-c3ccc4nc(C)n(C(C)C)c4n3)c12] 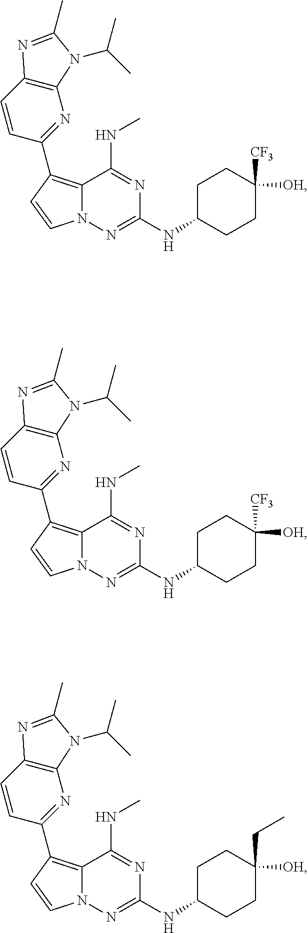 [SMILES: CNc1nc(N[C@H]2CC[C@](O)(C(F)(F)F)CC2)nn2ccc(-c3ccc4nc(C)n(C(C)C)c4n3)c12 ; CNc1nc(N[C@H]2CC[C@@](O)(C(F)(F)F)CC2)nn2ccc(-c3ccc4nc(C)n(C(C)C)c4n3)c12 ; CC[C@]1(O)CC[C@@H](Nc2nc(NC)c3c(-c4ccc5nc(C)n(C(C)C)c5n4)ccn3n2)CC1] 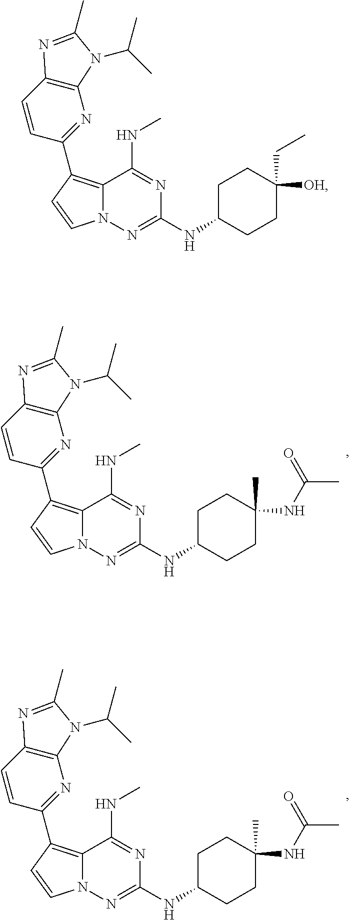 [SMILES: CC[C@]1(O)CC[C@H](Nc2nc(NC)c3c(-c4ccc5nc(C)n(C(C)C)c5n4)ccn3n2)CC1 ; CNc1nc(N[C@H]2CC[C@@](C)(NC(C)=O)CC2)nn2ccc(-c3ccc4nc(C)n(C(C)C)c4n3)c12 ; CNc1nc(N[C@H]2CC[C@](C)(NC(C)=O)CC2)nn2ccc(-c3ccc4nc(C)n(C(C)C)c4n3)c12] 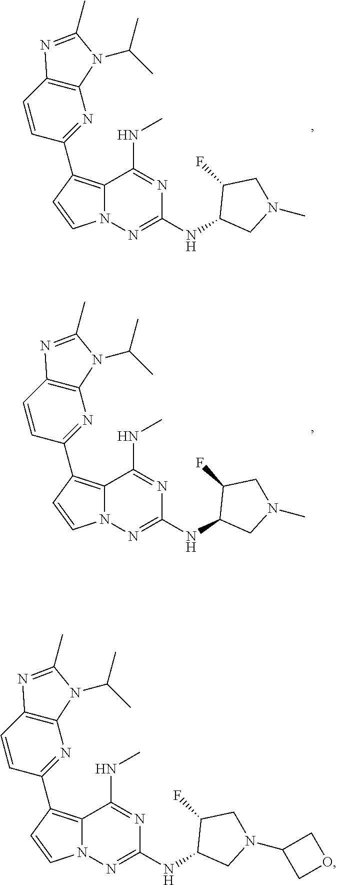 [SMILES: CNc1nc(N[C@H]2CN(C)C[C@H]2F)nn2ccc(-c3ccc4nc(C)n(C(C)C)c4n3)c12 ; CNc1nc(N[C@@H]2CN(C)C[C@@H]2F)nn2ccc(-c3ccc4nc(C)n(C(C)C)c4n3)c12 ; CNc1nc(N[C@H]2CN(C3COC3)C[C@H]2F)nn2ccc(-c3ccc4nc(C)n(C(C)C)c4n3)c12] 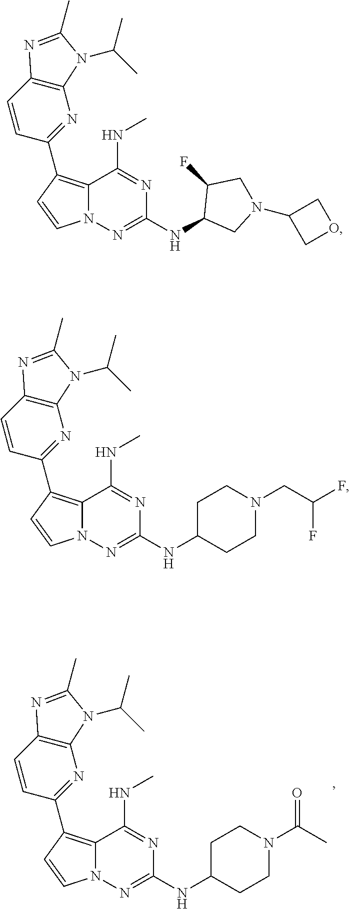 [SMILES: CNc1nc(N[C@@H]2CN(C3COC3)C[C@@H]2F)nn2ccc(-c3ccc4nc(C)n(C(C)C)c4n3)c12 ; CNc1nc(NC2CCN(CC(F)F)CC2)nn2ccc(-c3ccc4nc(C)n(C(C)C)c4n3)c12 ; CNc1nc(NC2CCN(C(C)=O)CC2)nn2ccc(-c3ccc4nc(C)n(C(C)C)c4n3)c12] 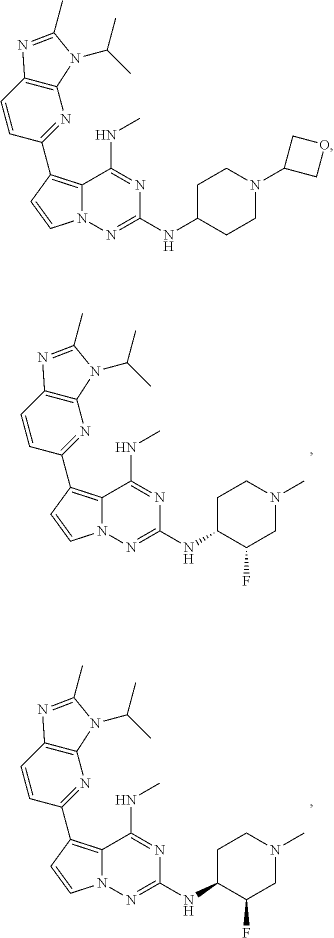 [SMILES: CNc1nc(NC2CCN(C3COC3)CC2)nn2ccc(-c3ccc4nc(C)n(C(C)C)c4n3)c12 ; CNc1nc(N[C@@H]2CCN(C)C[C@@H]2F)nn2ccc(-c3ccc4nc(C)n(C(C)C)c4n3)c12 ; CNc1nc(N[C@H]2CCN(C)C[C@H]2F)nn2ccc(-c3ccc4nc(C)n(C(C)C)c4n3)c12] 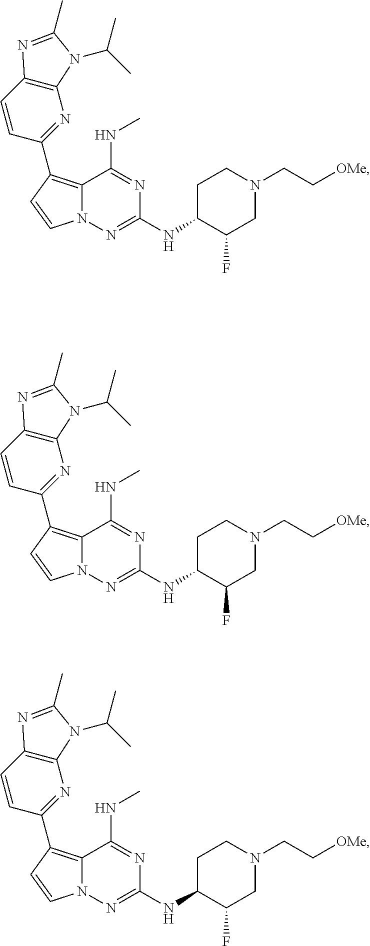 [SMILES: CNc1nc(N[C@@H]2CCN(CCOC)C[C@@H]2F)nn2ccc(-c3ccc4nc(C)n(C(C)C)c4n3)c12 ; CNc1nc(N[C@@H]2CCN(CCOC)C[C@H]2F)nn2ccc(-c3ccc4nc(C)n(C(C)C)c4n3)c12 ; CNc1nc(N[C@H]2CCN(CCOC)C[C@@H]2F)nn2ccc(-c3ccc4nc(C)n(C(C)C)c4n3)c12] 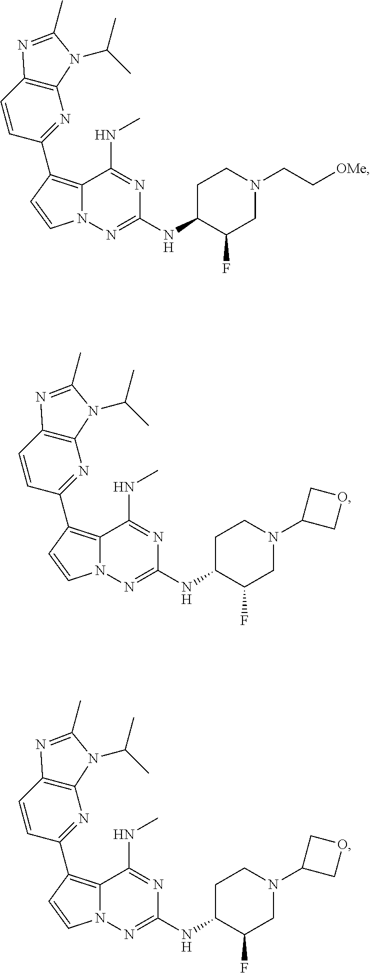 [SMILES: CNc1nc(N[C@H]2CCN(CCOC)C[C@H]2F)nn2ccc(-c3ccc4nc(C)n(C(C)C)c4n3)c12 ; CNc1nc(N[C@@H]2CCN(C3COC3)C[C@@H]2F)nn2ccc(-c3ccc4nc(C)n(C(C)C)c4n3)c12 ; CNc1nc(N[C@@H]2CCN(C3COC3)C[C@H]2F)nn2ccc(-c3ccc4nc(C)n(C(C)C)c4n3)c12] 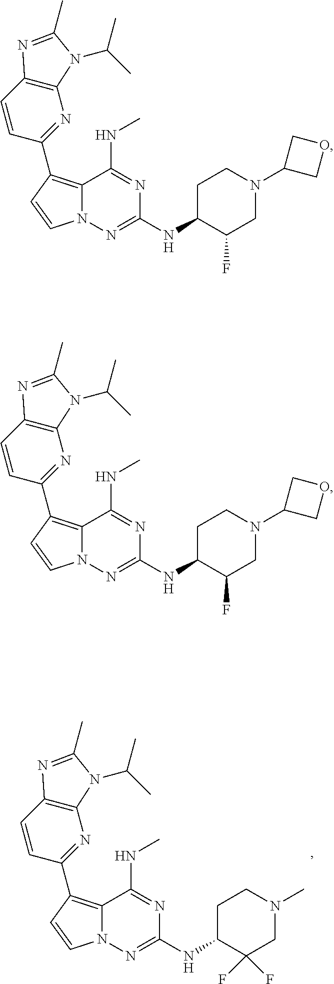 [SMILES: CNc1nc(N[C@H]2CCN(C3COC3)C[C@@H]2F)nn2ccc(-c3ccc4nc(C)n(C(C)C)c4n3)c12 ; CNc1nc(N[C@H]2CCN(C3COC3)C[C@H]2F)nn2ccc(-c3ccc4nc(C)n(C(C)C)c4n3)c12 ; CNc1nc(N[C@@H]2CCN(C)CC2(F)F)nn2ccc(-c3ccc4nc(C)n(C(C)C)c4n3)c12] 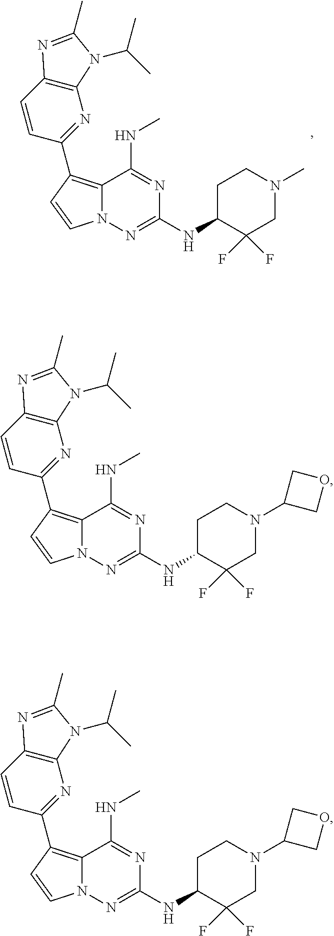 [SMILES: CNc1nc(N[C@H]2CCN(C)CC2(F)F)nn2ccc(-c3ccc4nc(C)n(C(C)C)c4n3)c12 ; CNc1nc(N[C@@H]2CCN(C3COC3)CC2(F)F)nn2ccc(-c3ccc4nc(C)n(C(C)C)c4n3)c12 ; CNc1nc(N[C@H]2CCN(C3COC3)CC2(F)F)nn2ccc(-c3ccc4nc(C)n(C(C)C)c4n3)c12] 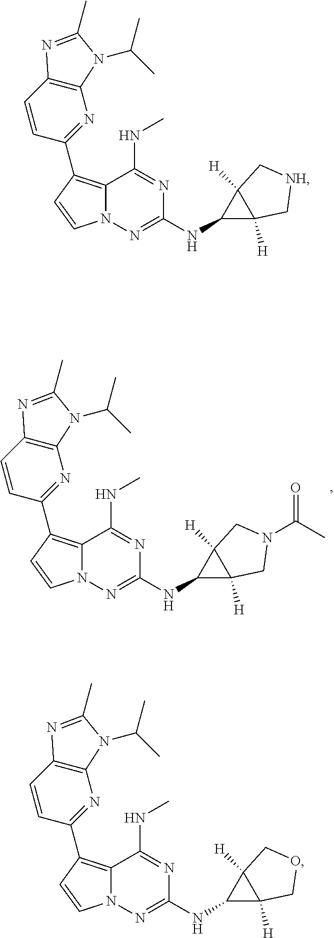 [SMILES: CNc1nc(N[C@@H]2[C@@H]3CNC[C@@H]32)nn2ccc(-c3ccc4nc(C)n(C(C)C)c4n3)c12 ; CNc1nc(N[C@@H]2[C@@H]3CN(C(C)=O)C[C@@H]32)nn2ccc(-c3ccc4nc(C)n(C(C)C)c4n3)c12 ; CNc1nc(N[C@H]2[C@@H]3COC[C@@H]32)nn2ccc(-c3ccc4nc(C)n(C(C)C)c4n3)c12] 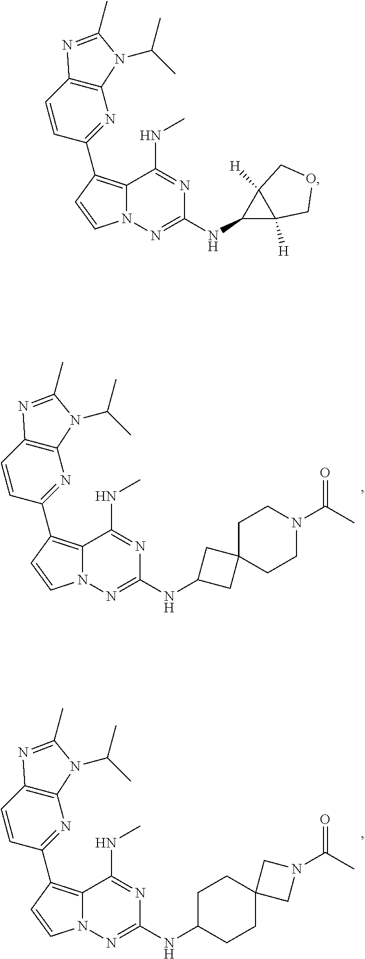 [SMILES: CNc1nc(N[C@@H]2[C@@H]3COC[C@@H]32)nn2ccc(-c3ccc4nc(C)n(C(C)C)c4n3)c12 ; CNc1nc(NC2CC3(CCN(C(C)=O)CC3)C2)nn2ccc(-c3ccc4nc(C)n(C(C)C)c4n3)c12 ; CNc1nc(NC2CCC3(CC2)CN(C(C)=O)C3)nn2ccc(-c3ccc4nc(C)n(C(C)C)c4n3)c12] 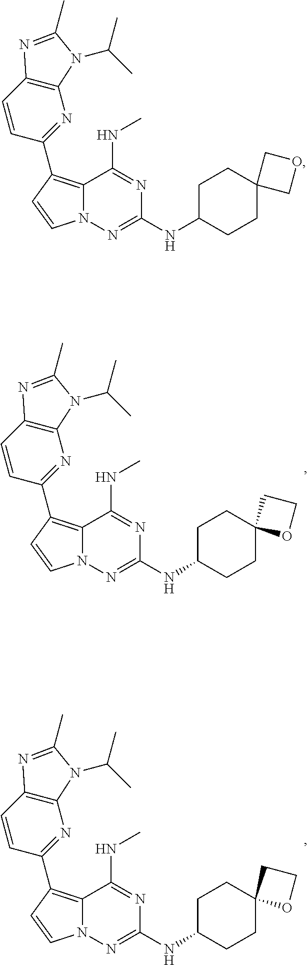 [SMILES: CNc1nc(NC2CCC3(CC2)COC3)nn2ccc(-c3ccc4nc(C)n(C(C)C)c4n3)c12 ; CNc1nc(N[C@H]2CC[C@@]3(CCO3)CC2)nn2ccc(-c3ccc4nc(C)n(C(C)C)c4n3)c12 ; CNc1nc(N[C@H]2CC[C@]3(CCO3)CC2)nn2ccc(-c3ccc4nc(C)n(C(C)C)c4n3)c12] 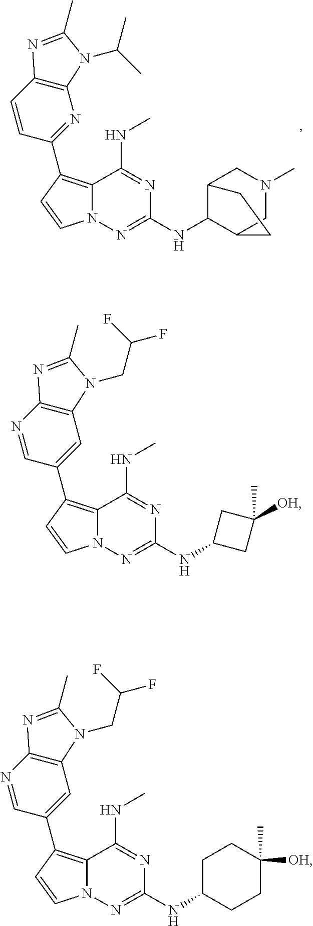 [SMILES: CNc1nc(NC2C3CCC2CN(C)C3)nn2ccc(-c3ccc4nc(C)n(C(C)C)c4n3)c12 ; CNc1nc(N[C@H]2C[C@](C)(O)C2)nn2ccc(-c3cnc4nc(C)n(CC(F)F)c4c3)c12 ; CNc1nc(N[C@H]2CC[C@](C)(O)CC2)nn2ccc(-c3cnc4nc(C)n(CC(F)F)c4c3)c12] 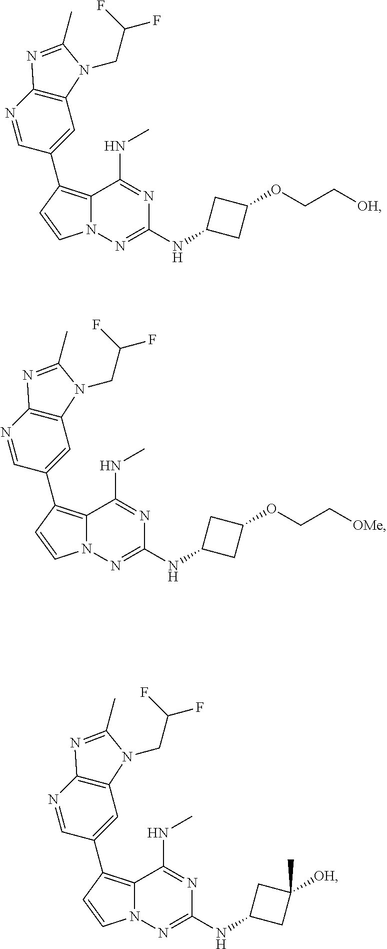 [SMILES: CNc1nc(N[C@H]2C[C@@H](OCCO)C2)nn2ccc(-c3cnc4nc(C)n(CC(F)F)c4c3)c12 ; CNc1nc(N[C@H]2C[C@@H](OCCOC)C2)nn2ccc(-c3cnc4nc(C)n(CC(F)F)c4c3)c12 ; CNc1nc(N[C@H]2C[C@@](C)(O)C2)nn2ccc(-c3cnc4nc(C)n(CC(F)F)c4c3)c12] 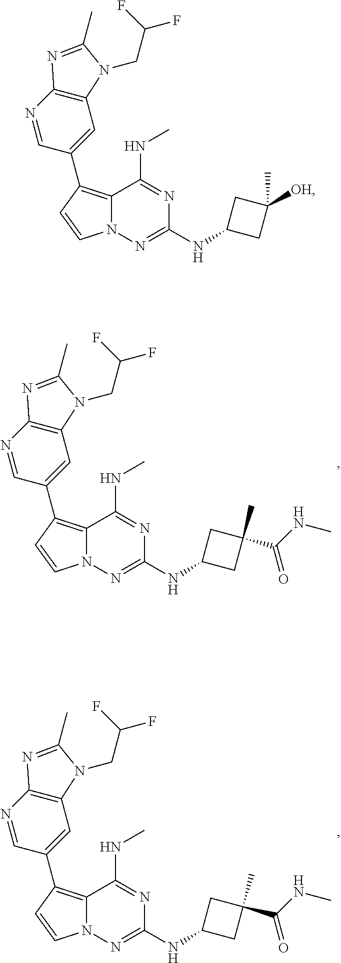 [SMILES: CNc1nc(N[C@H]2C[C@](C)(O)C2)nn2ccc(-c3cnc4nc(C)n(CC(F)F)c4c3)c12 ; CNc1nc(N[C@H]2C[C@@](C)(C(=O)NC)C2)nn2ccc(-c3cnc4nc(C)n(CC(F)F)c4c3)c12 ; CNc1nc(N[C@H]2C[C@](C)(C(=O)NC)C2)nn2ccc(-c3cnc4nc(C)n(CC(F)F)c4c3)c12] 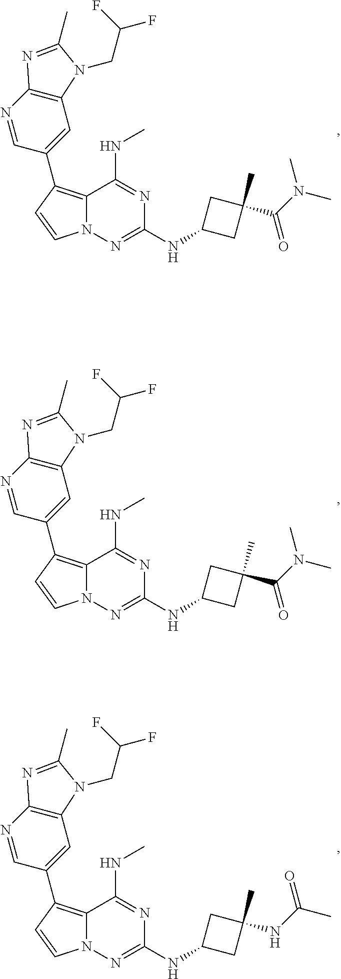 [SMILES: CNc1nc(N[C@H]2C[C@@](C)(C(=O)N(C)C)C2)nn2ccc(-c3cnc4nc(C)n(CC(F)F)c4c3)c12 ; CNc1nc(N[C@H]2C[C@](C)(C(=O)N(C)C)C2)nn2ccc(-c3cnc4nc(C)n(CC(F)F)c4c3)c12 ; CNc1nc(N[C@H]2C[C@@](C)(NC(C)=O)C2)nn2ccc(-c3cnc4nc(C)n(CC(F)F)c4c3)c12] 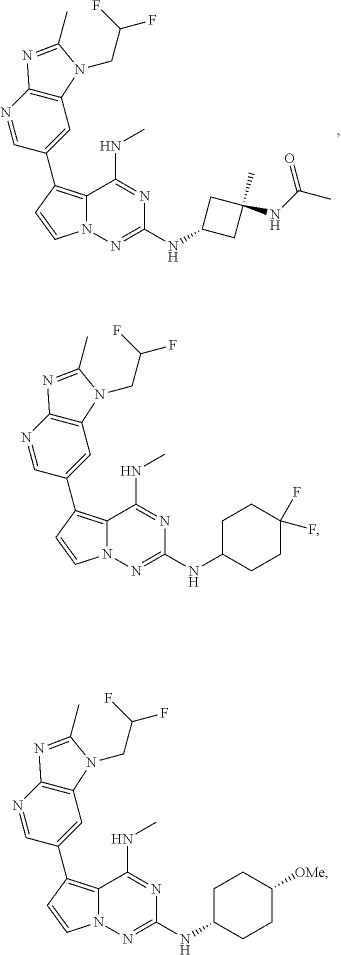 [SMILES: CNc1nc(N[C@H]2C[C@](C)(NC(C)=O)C2)nn2ccc(-c3cnc4nc(C)n(CC(F)F)c4c3)c12 ; CNc1nc(NC2CCC(F)(F)CC2)nn2ccc(-c3cnc4nc(C)n(CC(F)F)c4c3)c12 ; CNc1nc(N[C@H]2CC[C@@H](OC)CC2)nn2ccc(-c3cnc4nc(C)n(CC(F)F)c4c3)c12] 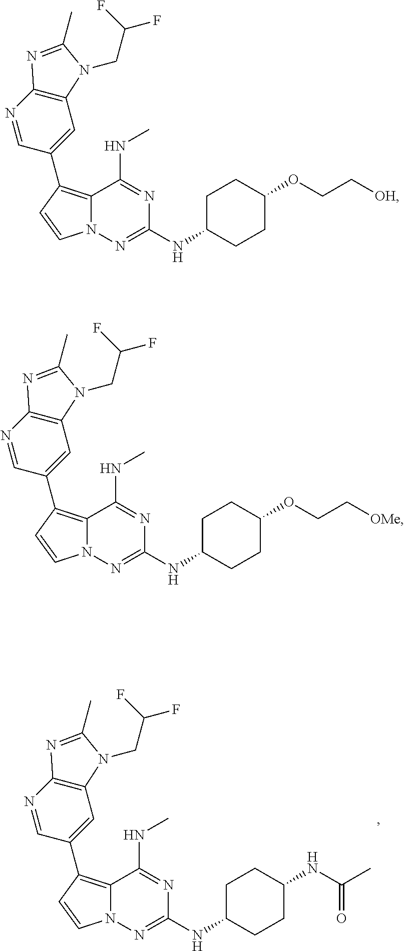 [SMILES: CNc1nc(N[C@H]2CC[C@@H](OCCO)CC2)nn2ccc(-c3cnc4nc(C)n(CC(F)F)c4c3)c12 ; CNc1nc(N[C@H]2CC[C@@H](OCCOC)CC2)nn2ccc(-c3cnc4nc(C)n(CC(F)F)c4c3)c12 ; CNc1nc(N[C@H]2CC[C@@H](NC(C)=O)CC2)nn2ccc(-c3cnc4nc(C)n(CC(F)F)c4c3)c12] 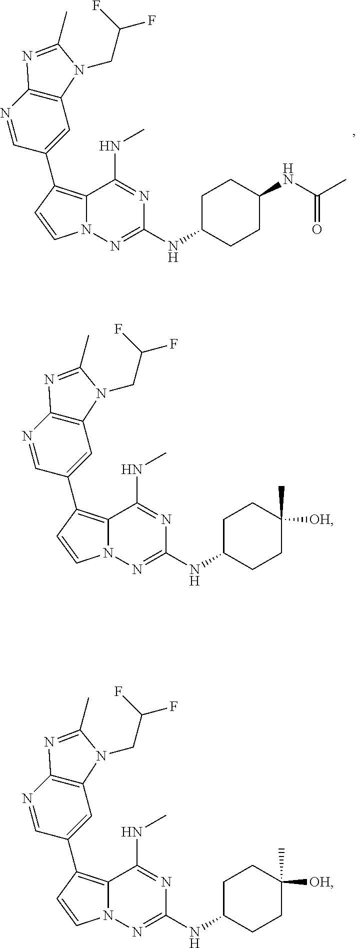 [SMILES: CNc1nc(N[C@H]2CC[C@H](NC(C)=O)CC2)nn2ccc(-c3cnc4nc(C)n(CC(F)F)c4c3)c12 ; CNc1nc(N[C@H]2CC[C@@](C)(O)CC2)nn2ccc(-c3cnc4nc(C)n(CC(F)F)c4c3)c12 ; CNc1nc(N[C@H]2CC[C@](C)(O)CC2)nn2ccc(-c3cnc4nc(C)n(CC(F)F)c4c3)c12] 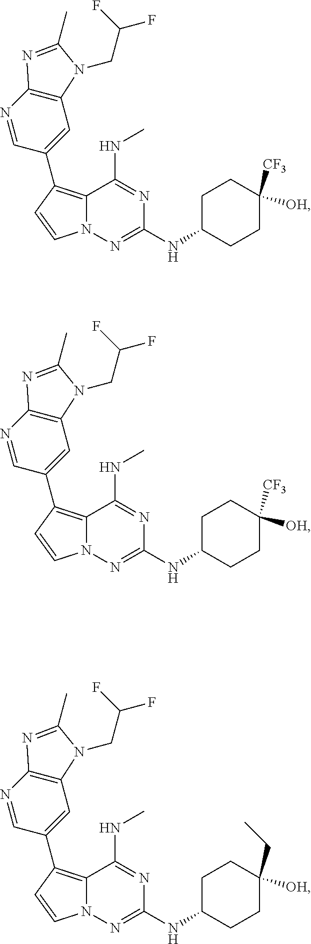 [SMILES: CNc1nc(N[C@H]2CC[C@](O)(C(F)(F)F)CC2)nn2ccc(-c3cnc4nc(C)n(CC(F)F)c4c3)c12 ; CNc1nc(N[C@H]2CC[C@@](O)(C(F)(F)F)CC2)nn2ccc(-c3cnc4nc(C)n(CC(F)F)c4c3)c12 ; CC[C@]1(O)CC[C@@H](Nc2nc(NC)c3c(-c4cnc5nc(C)n(CC(F)F)c5c4)ccn3n2)CC1] 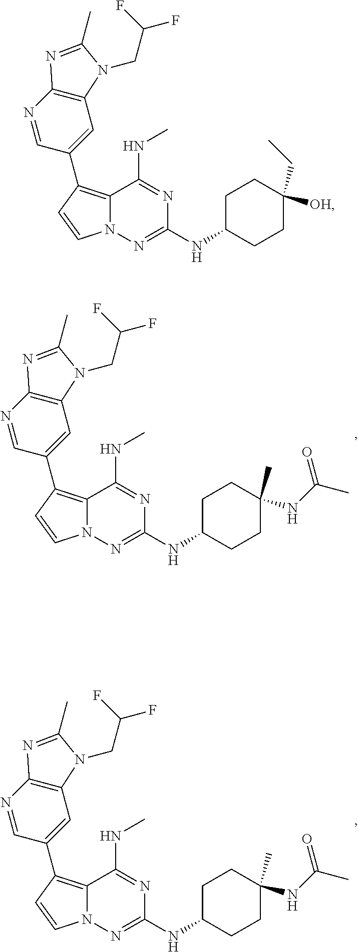 [SMILES: CC[C@]1(O)CC[C@H](Nc2nc(NC)c3c(-c4cnc5nc(C)n(CC(F)F)c5c4)ccn3n2)CC1 ; CNc1nc(N[C@H]2CC[C@@](C)(NC(C)=O)CC2)nn2ccc(-c3cnc4nc(C)n(CC(F)F)c4c3)c12 ; CNc1nc(N[C@H]2CC[C@](C)(NC(C)=O)CC2)nn2ccc(-c3cnc4nc(C)n(CC(F)F)c4c3)c12] 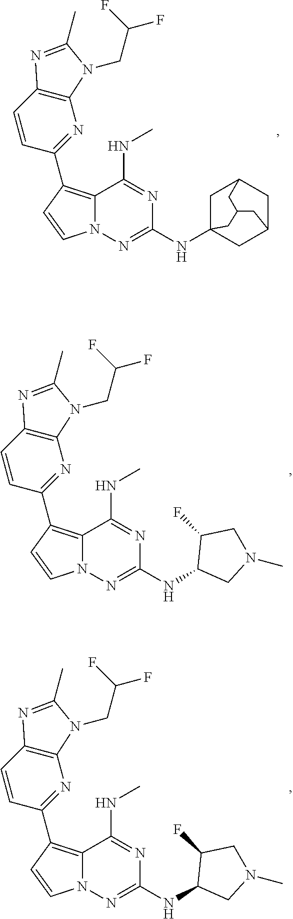 [SMILES: CNc1nc(NC23CC4CC(CC(C4)C2)C3)nn2ccc(-c3ccc4nc(C)n(CC(F)F)c4n3)c12 ; CNc1nc(N[C@H]2CN(C)C[C@H]2F)nn2ccc(-c3ccc4nc(C)n(CC(F)F)c4n3)c12 ; CNc1nc(N[C@@H]2CN(C)C[C@@H]2F)nn2ccc(-c3ccc4nc(C)n(CC(F)F)c4n3)c12] 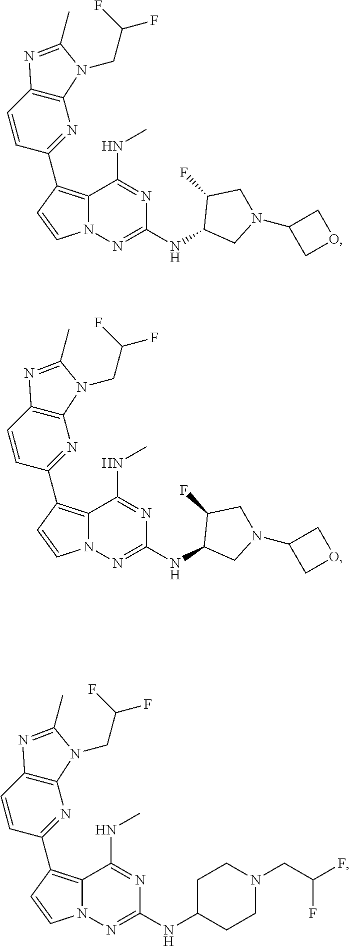 [SMILES: CNc1nc(N[C@H]2CN(C3COC3)C[C@H]2F)nn2ccc(-c3ccc4nc(C)n(CC(F)F)c4n3)c12 ; CNc1nc(N[C@@H]2CN(C3COC3)C[C@@H]2F)nn2ccc(-c3ccc4nc(C)n(CC(F)F)c4n3)c12 ; CNc1nc(NC2CCN(CC(F)F)CC2)nn2ccc(-c3ccc4nc(C)n(CC(F)F)c4n3)c12] 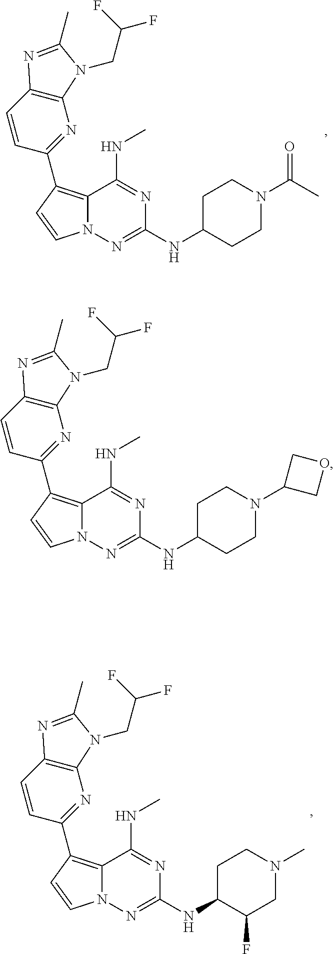 [SMILES: CNc1nc(NC2CCN(C(C)=O)CC2)nn2ccc(-c3ccc4nc(C)n(CC(F)F)c4n3)c12 ; CNc1nc(NC2CCN(C3COC3)CC2)nn2ccc(-c3ccc4nc(C)n(CC(F)F)c4n3)c12 ; CNc1nc(N[C@H]2CCN(C)C[C@H]2F)nn2ccc(-c3ccc4nc(C)n(CC(F)F)c4n3)c12] 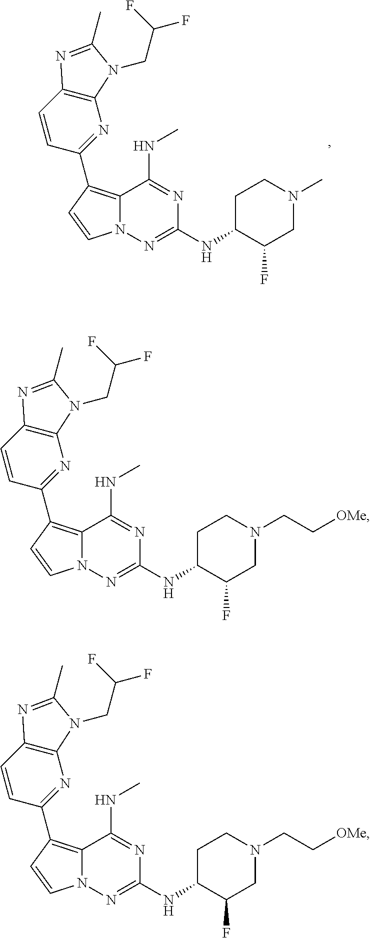 [SMILES: CNc1nc(N[C@@H]2CCN(C)C[C@@H]2F)nn2ccc(-c3ccc4nc(C)n(CC(F)F)c4n3)c12 ; CNc1nc(N[C@@H]2CCN(CCOC)C[C@@H]2F)nn2ccc(-c3ccc4nc(C)n(CC(F)F)c4n3)c12 ; CNc1nc(N[C@@H]2CCN(CCOC)C[C@H]2F)nn2ccc(-c3ccc4nc(C)n(CC(F)F)c4n3)c12] 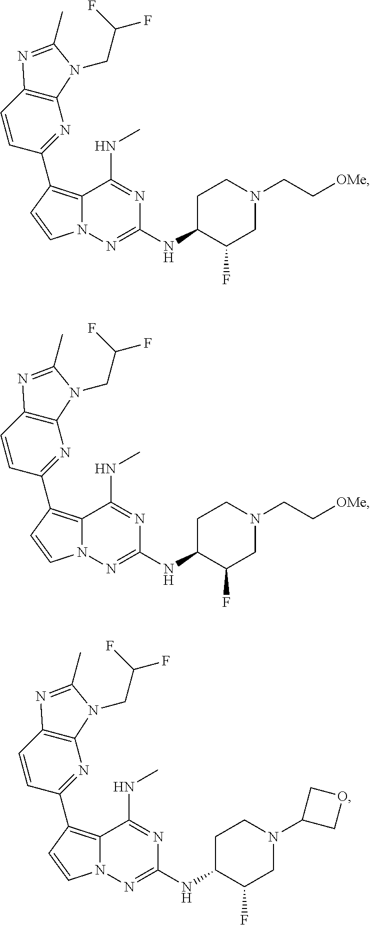 [SMILES: CNc1nc(N[C@H]2CCN(CCOC)C[C@@H]2F)nn2ccc(-c3ccc4nc(C)n(CC(F)F)c4n3)c12 ; CNc1nc(N[C@H]2CCN(CCOC)C[C@H]2F)nn2ccc(-c3ccc4nc(C)n(CC(F)F)c4n3)c12 ; CNc1nc(N[C@@H]2CCN(C3COC3)C[C@@H]2F)nn2ccc(-c3ccc4nc(C)n(CC(F)F)c4n3)c12] 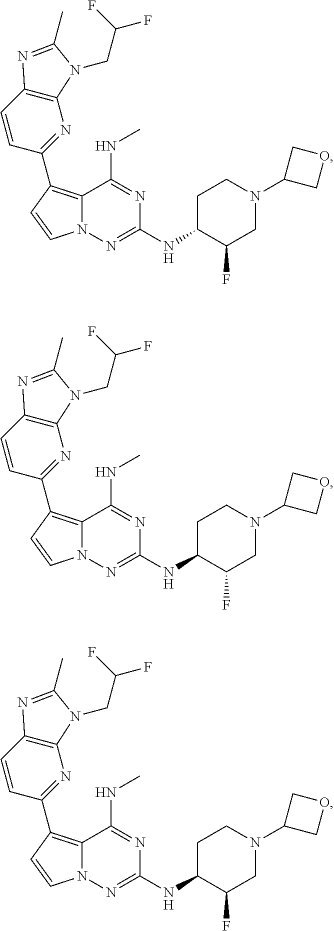 [SMILES: CNc1nc(N[C@@H]2CCN(C3COC3)C[C@H]2F)nn2ccc(-c3ccc4nc(C)n(CC(F)F)c4n3)c12 ; CNc1nc(N[C@H]2CCN(C3COC3)C[C@@H]2F)nn2ccc(-c3ccc4nc(C)n(CC(F)F)c4n3)c12 ; CNc1nc(N[C@H]2CCN(C3COC3)C[C@H]2F)nn2ccc(-c3ccc4nc(C)n(CC(F)F)c4n3)c12] 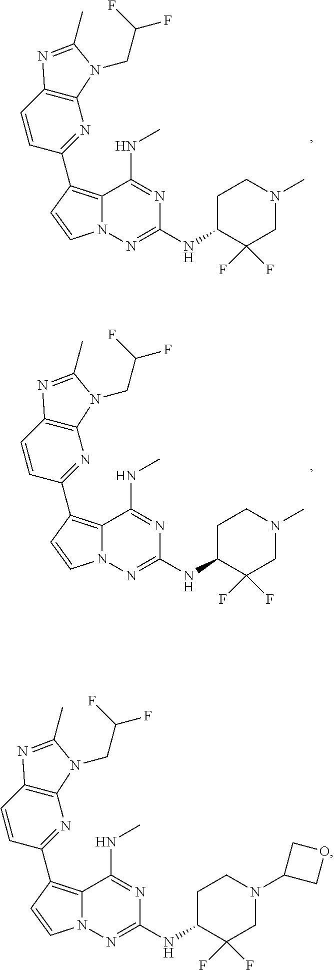 [SMILES: CNc1nc(N[C@@H]2CCN(C)CC2(F)F)nn2ccc(-c3ccc4nc(C)n(CC(F)F)c4n3)c12 ; CNc1nc(N[C@H]2CCN(C)CC2(F)F)nn2ccc(-c3ccc4nc(C)n(CC(F)F)c4n3)c12 ; CNc1nc(N[C@@H]2CCN(C3COC3)CC2(F)F)nn2ccc(-c3ccc4nc(C)n(CC(F)F)c4n3)c12] 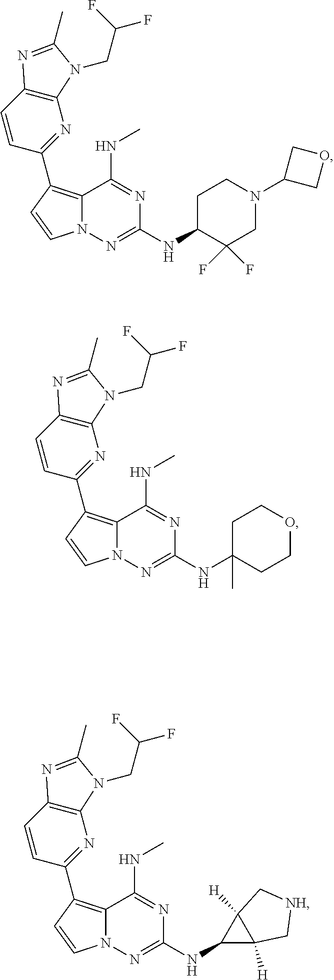 [SMILES: CNc1nc(N[C@H]2CCN(C3COC3)CC2(F)F)nn2ccc(-c3ccc4nc(C)n(CC(F)F)c4n3)c12 ; CNc1nc(NC2(C)CCOCC2)nn2ccc(-c3ccc4nc(C)n(CC(F)F)c4n3)c12 ; CNc1nc(N[C@@H]2[C@@H]3CNC[C@@H]32)nn2ccc(-c3ccc4nc(C)n(CC(F)F)c4n3)c12] 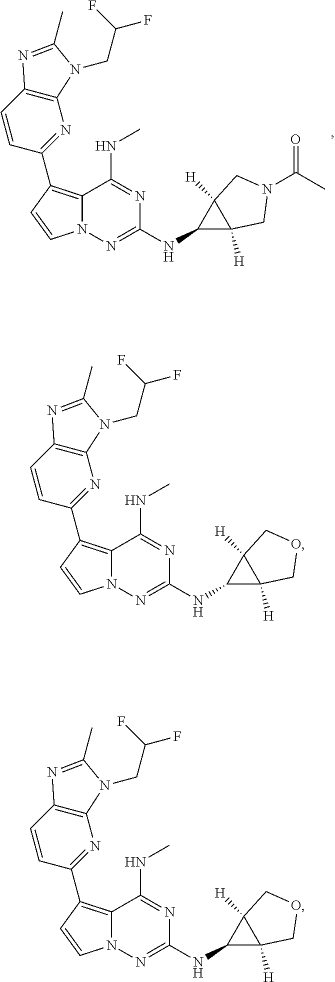 [SMILES: CNc1nc(N[C@@H]2[C@@H]3CN(C(C)=O)C[C@@H]32)nn2ccc(-c3ccc4nc(C)n(CC(F)F)c4n3)c12 ; CNc1nc(N[C@H]2[C@@H]3COC[C@@H]32)nn2ccc(-c3ccc4nc(C)n(CC(F)F)c4n3)c12 ; CNc1nc(N[C@@H]2[C@@H]3COC[C@@H]32)nn2ccc(-c3ccc4nc(C)n(CC(F)F)c4n3)c12] 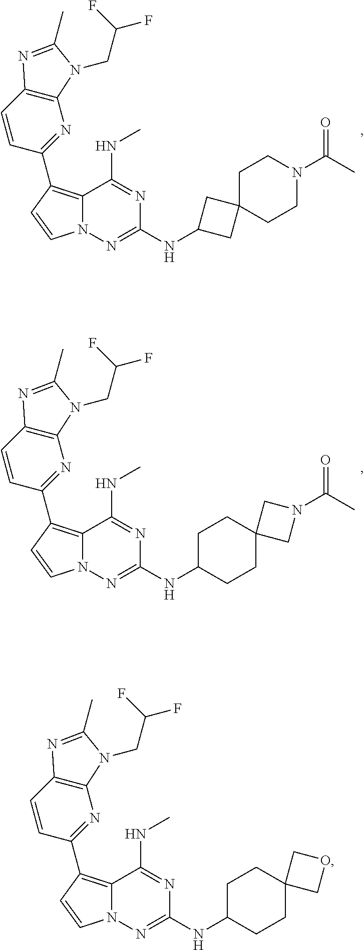 [SMILES: CNc1nc(NC2CC3(CCN(C(C)=O)CC3)C2)nn2ccc(-c3ccc4nc(C)n(CC(F)F)c4n3)c12 ; CNc1nc(NC2CCC3(CC2)CN(C(C)=O)C3)nn2ccc(-c3ccc4nc(C)n(CC(F)F)c4n3)c12 ; CNc1nc(NC2CCC3(CC2)COC3)nn2ccc(-c3ccc4nc(C)n(CC(F)F)c4n3)c12] 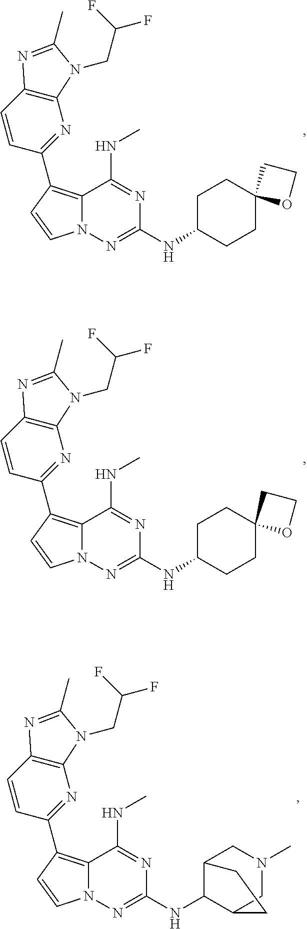 [SMILES: CNc1nc(N[C@H]2CC[C@@]3(CCO3)CC2)nn2ccc(-c3ccc4nc(C)n(CC(F)F)c4n3)c12 ; CNc1nc(N[C@H]2CC[C@]3(CCO3)CC2)nn2ccc(-c3ccc4nc(C)n(CC(F)F)c4n3)c12 ; CNc1nc(NC2C3CCC2CN(C)C3)nn2ccc(-c3ccc4nc(C)n(CC(F)F)c4n3)c12] 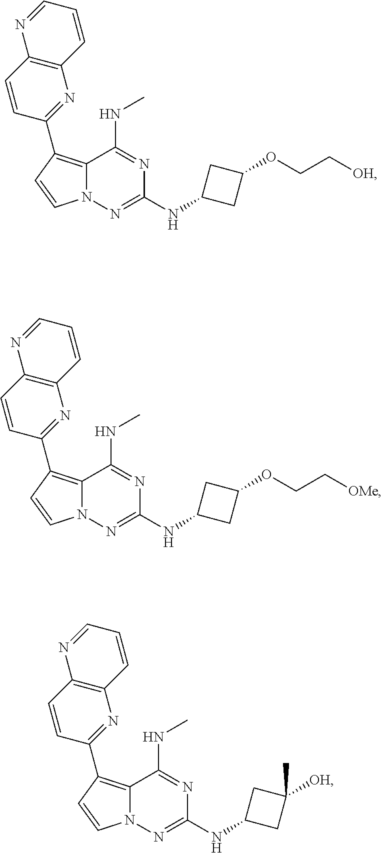 [SMILES: CNc1nc(N[C@H]2C[C@@H](OCCO)C2)nn2ccc(-c3ccc4ncccc4n3)c12 ; CNc1nc(N[C@H]2C[C@@H](OCCOC)C2)nn2ccc(-c3ccc4ncccc4n3)c12 ; CNc1nc(N[C@H]2C[C@@](C)(O)C2)nn2ccc(-c3ccc4ncccc4n3)c12] 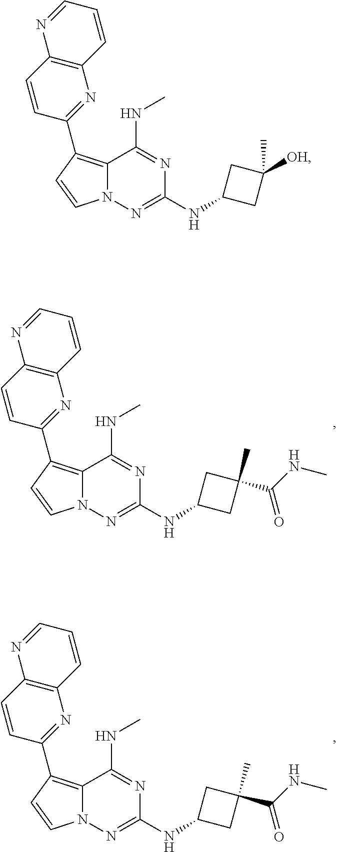 [SMILES: CNc1nc(N[C@H]2C[C@](C)(O)C2)nn2ccc(-c3ccc4ncccc4n3)c12 ; CNc1nc(N[C@H]2C[C@@](C)(C(=O)NC)C2)nn2ccc(-c3ccc4ncccc4n3)c12 ; CNc1nc(N[C@H]2C[C@](C)(C(=O)NC)C2)nn2ccc(-c3ccc4ncccc4n3)c12] 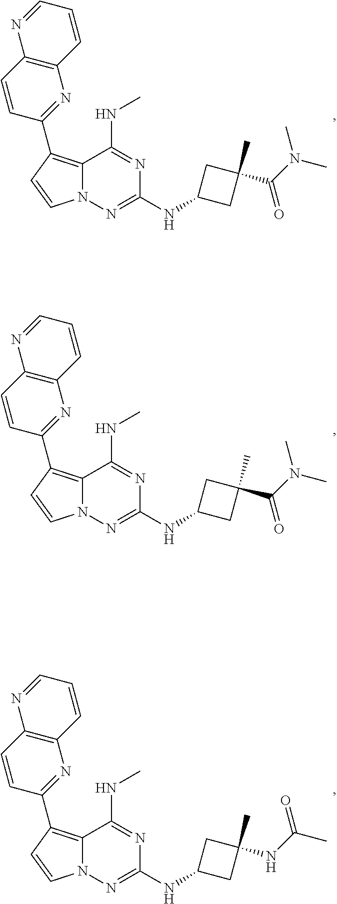 [SMILES: CNc1nc(N[C@H]2C[C@@](C)(C(=O)N(C)C)C2)nn2ccc(-c3ccc4ncccc4n3)c12 ; CNc1nc(N[C@H]2C[C@](C)(C(=O)N(C)C)C2)nn2ccc(-c3ccc4ncccc4n3)c12 ; CNc1nc(N[C@H]2C[C@@](C)(NC(C)=O)C2)nn2ccc(-c3ccc4ncccc4n3)c12] 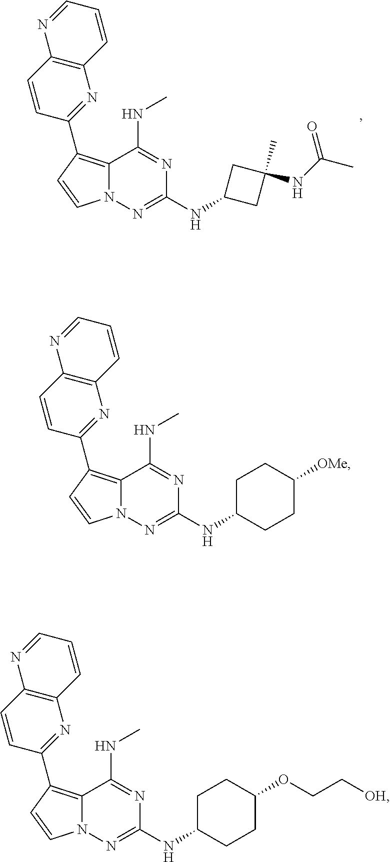 [SMILES: CNc1nc(N[C@H]2C[C@](C)(NC(C)=O)C2)nn2ccc(-c3ccc4ncccc4n3)c12 ; CNc1nc(N[C@H]2CC[C@@H](OC)CC2)nn2ccc(-c3ccc4ncccc4n3)c12 ; CNc1nc(N[C@H]2CC[C@@H](OCCO)CC2)nn2ccc(-c3ccc4ncccc4n3)c12] 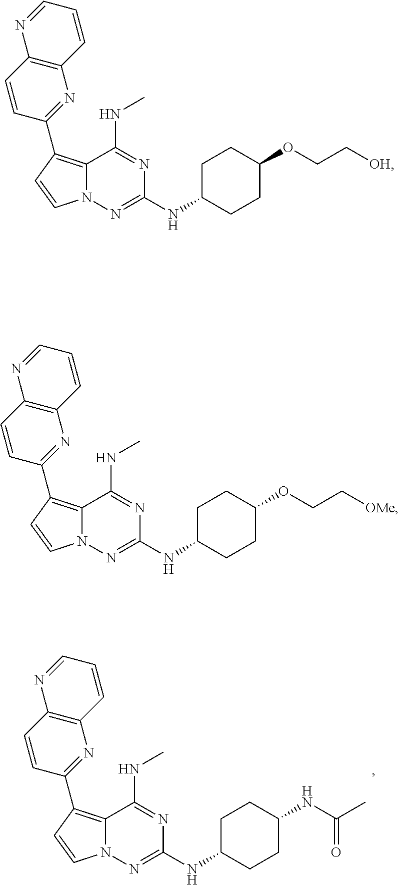 [SMILES: CNc1nc(N[C@H]2CC[C@H](OCCO)CC2)nn2ccc(-c3ccc4ncccc4n3)c12 ; CNc1nc(N[C@H]2CC[C@@H](OCCOC)CC2)nn2ccc(-c3ccc4ncccc4n3)c12 ; CNc1nc(N[C@H]2CC[C@@H](NC(C)=O)CC2)nn2ccc(-c3ccc4ncccc4n3)c12] 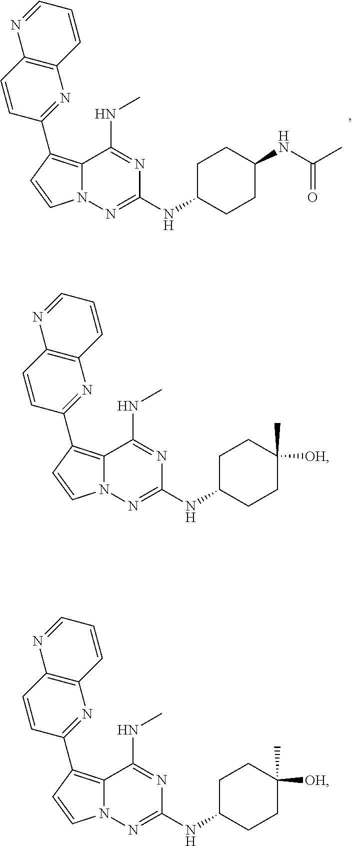 [SMILES: CNc1nc(N[C@H]2CC[C@H](NC(C)=O)CC2)nn2ccc(-c3ccc4ncccc4n3)c12 ; CNc1nc(N[C@H]2CC[C@@](C)(O)CC2)nn2ccc(-c3ccc4ncccc4n3)c12 ; CNc1nc(N[C@H]2CC[C@](C)(O)CC2)nn2ccc(-c3ccc4ncccc4n3)c12] 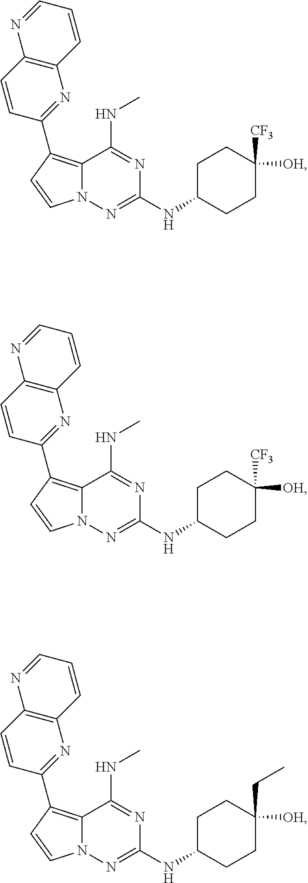 [SMILES: CNc1nc(N[C@H]2CC[C@](O)(C(F)(F)F)CC2)nn2ccc(-c3ccc4ncccc4n3)c12 ; CNc1nc(N[C@H]2CC[C@@](O)(C(F)(F)F)CC2)nn2ccc(-c3ccc4ncccc4n3)c12 ; CC[C@]1(O)CC[C@@H](Nc2nc(NC)c3c(-c4ccc5ncccc5n4)ccn3n2)CC1] 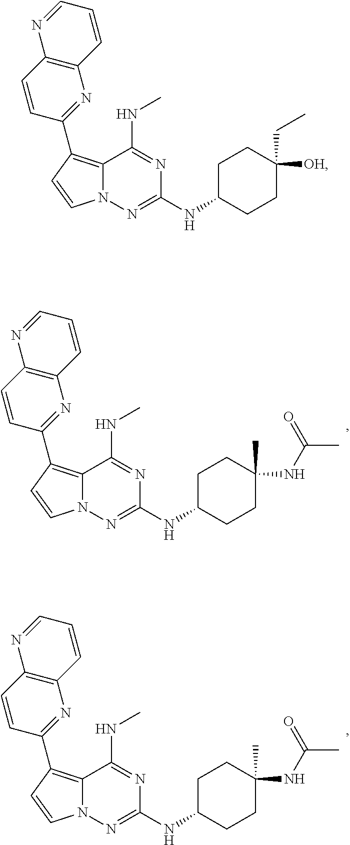 [SMILES: CC[C@]1(O)CC[C@H](Nc2nc(NC)c3c(-c4ccc5ncccc5n4)ccn3n2)CC1 ; CNc1nc(N[C@H]2CC[C@@](C)(NC(C)=O)CC2)nn2ccc(-c3ccc4ncccc4n3)c12 ; CNc1nc(N[C@H]2CC[C@](C)(NC(C)=O)CC2)nn2ccc(-c3ccc4ncccc4n3)c12] 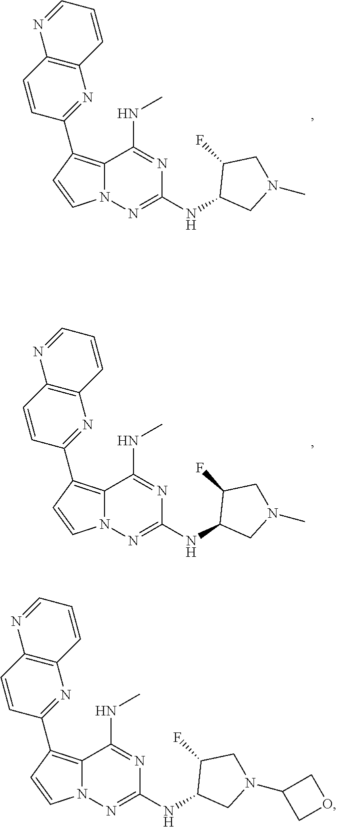 [SMILES: CNc1nc(N[C@H]2CN(C)C[C@H]2F)nn2ccc(-c3ccc4ncccc4n3)c12 ; CNc1nc(N[C@@H]2CN(C)C[C@@H]2F)nn2ccc(-c3ccc4ncccc4n3)c12 ; CNc1nc(N[C@H]2CN(C3COC3)C[C@H]2F)nn2ccc(-c3ccc4ncccc4n3)c12] 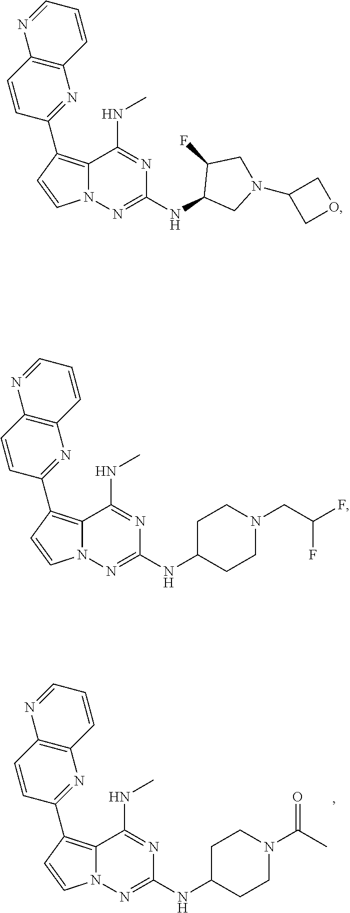 [SMILES: CNc1nc(N[C@@H]2CN(C3COC3)C[C@@H]2F)nn2ccc(-c3ccc4ncccc4n3)c12 ; CNc1nc(NC2CCN(CC(F)F)CC2)nn2ccc(-c3ccc4ncccc4n3)c12 ; CNc1nc(NC2CCN(C(C)=O)CC2)nn2ccc(-c3ccc4ncccc4n3)c12] 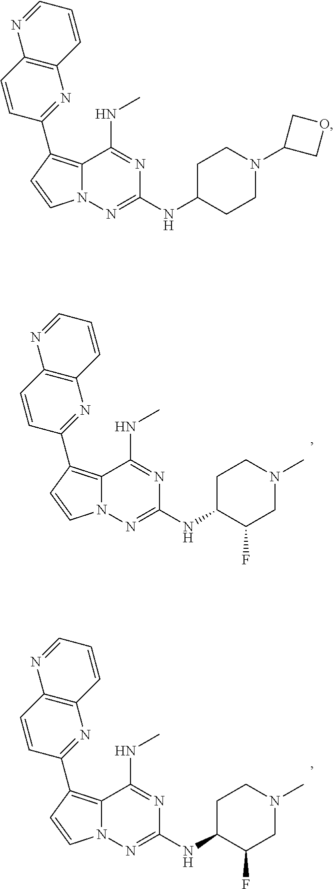 [SMILES: CNc1nc(NC2CCN(C3COC3)CC2)nn2ccc(-c3ccc4ncccc4n3)c12 ; CNc1nc(N[C@@H]2CCN(C)C[C@@H]2F)nn2ccc(-c3ccc4ncccc4n3)c12 ; CNc1nc(N[C@H]2CCN(C)C[C@H]2F)nn2ccc(-c3ccc4ncccc4n3)c12] 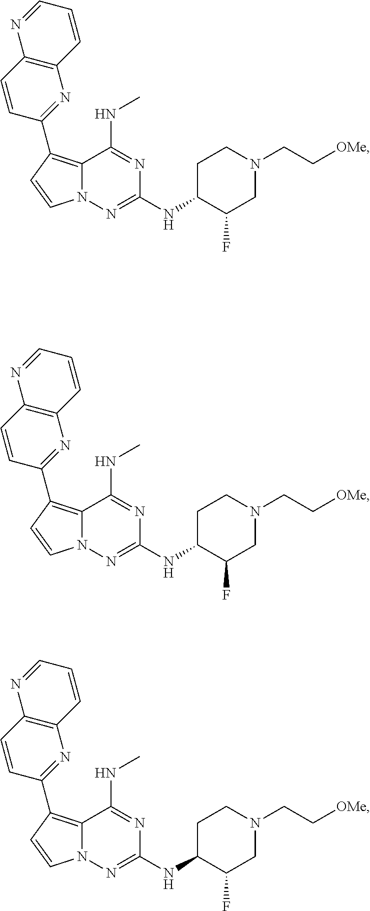 [SMILES: CNc1nc(N[C@@H]2CCN(CCOC)C[C@@H]2F)nn2ccc(-c3ccc4ncccc4n3)c12 ; CNc1nc(N[C@@H]2CCN(CCOC)C[C@H]2F)nn2ccc(-c3ccc4ncccc4n3)c12 ; CNc1nc(N[C@H]2CCN(CCOC)C[C@@H]2F)nn2ccc(-c3ccc4ncccc4n3)c12] 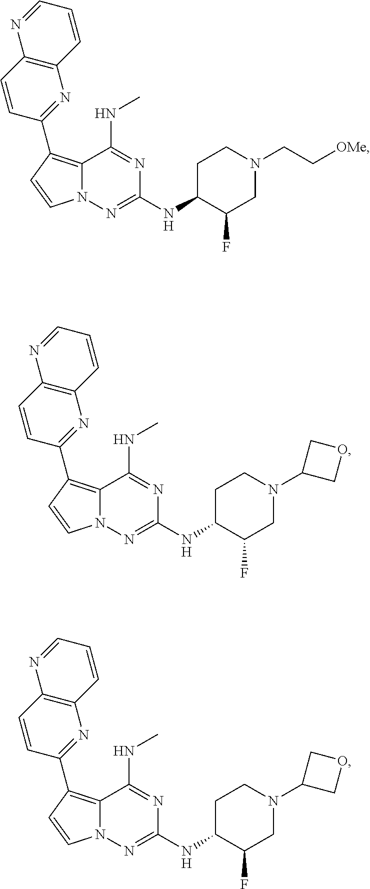 [SMILES: CNc1nc(N[C@H]2CCN(CCOC)C[C@H]2F)nn2ccc(-c3ccc4ncccc4n3)c12 ; CNc1nc(N[C@@H]2CCN(C3COC3)C[C@@H]2F)nn2ccc(-c3ccc4ncccc4n3)c12 ; CNc1nc(N[C@@H]2CCN(C3COC3)C[C@H]2F)nn2ccc(-c3ccc4ncccc4n3)c12] 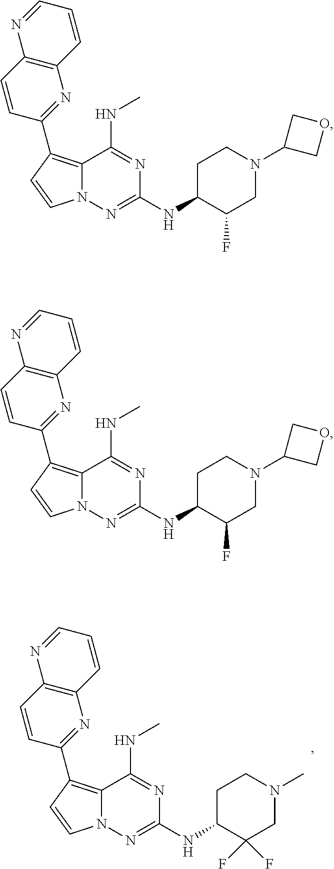 [SMILES: CNc1nc(N[C@H]2CCN(C3COC3)C[C@@H]2F)nn2ccc(-c3ccc4ncccc4n3)c12 ; CNc1nc(N[C@H]2CCN(C3COC3)C[C@H]2F)nn2ccc(-c3ccc4ncccc4n3)c12 ; CNc1nc(N[C@@H]2CCN(C)CC2(F)F)nn2ccc(-c3ccc4ncccc4n3)c12] 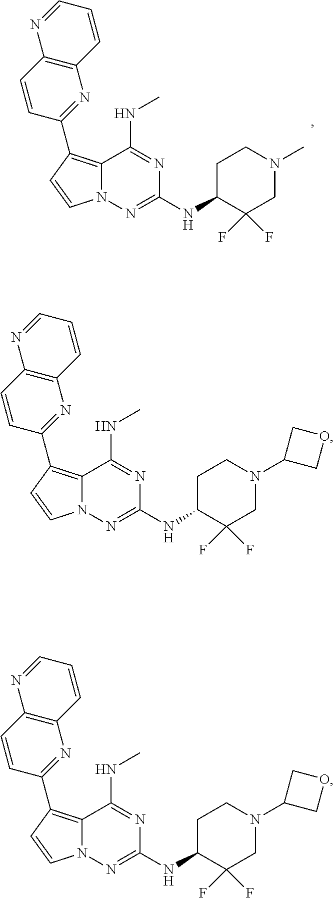 [SMILES: CNc1nc(N[C@H]2CCN(C)CC2(F)F)nn2ccc(-c3ccc4ncccc4n3)c12 ; CNc1nc(N[C@@H]2CCN(C3COC3)CC2(F)F)nn2ccc(-c3ccc4ncccc4n3)c12 ; CNc1nc(N[C@H]2CCN(C3COC3)CC2(F)F)nn2ccc(-c3ccc4ncccc4n3)c12] 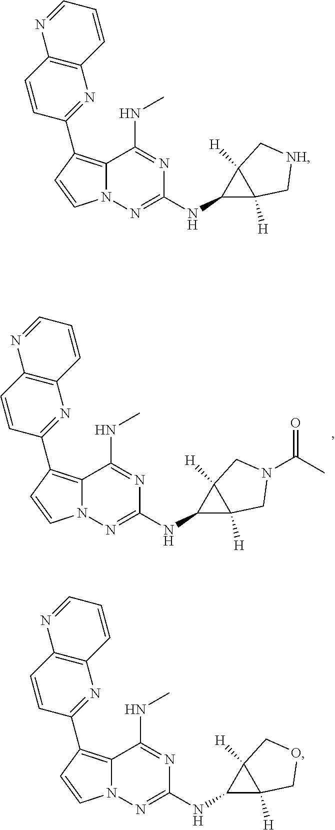 [SMILES: CNc1nc(N[C@@H]2[C@@H]3CNC[C@@H]32)nn2ccc(-c3ccc4ncccc4n3)c12 ; CNc1nc(N[C@@H]2[C@@H]3CN(C(C)=O)C[C@@H]32)nn2ccc(-c3ccc4ncccc4n3)c12 ; CNc1nc(N[C@H]2[C@@H]3COC[C@@H]32)nn2ccc(-c3ccc4ncccc4n3)c12] 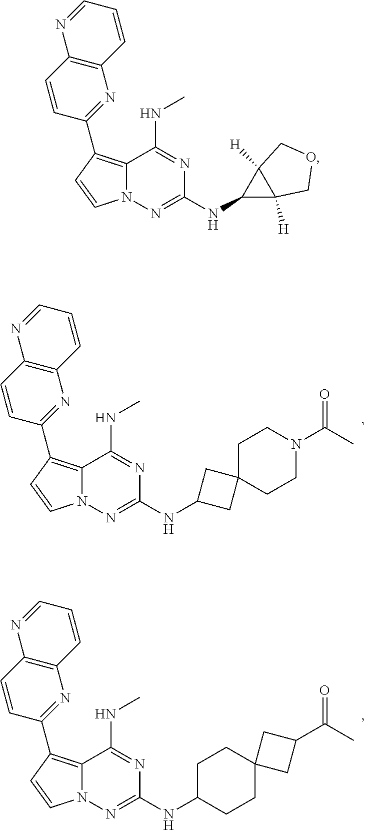 [SMILES: CNc1nc(N[C@@H]2[C@@H]3COC[C@@H]32)nn2ccc(-c3ccc4ncccc4n3)c12 ; CNc1nc(NC2CC3(CCN(C(C)=O)CC3)C2)nn2ccc(-c3ccc4ncccc4n3)c12 ; CNc1nc(NC2CCC3(CC2)CC(C(C)=O)C3)nn2ccc(-c3ccc4ncccc4n3)c12] 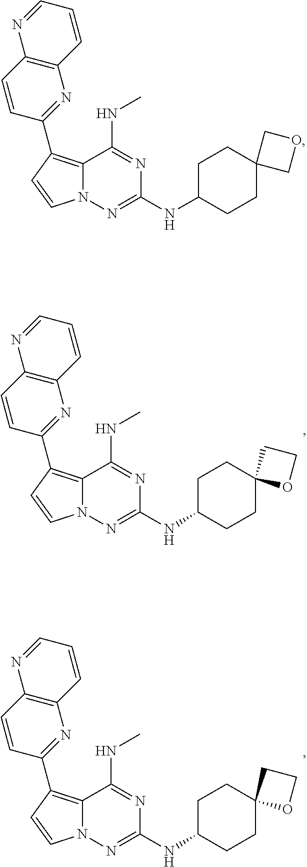 [SMILES: CNc1nc(NC2CCC3(CC2)COC3)nn2ccc(-c3ccc4ncccc4n3)c12 ; CNc1nc(N[C@H]2CC[C@@]3(CCO3)CC2)nn2ccc(-c3ccc4ncccc4n3)c12 ; CNc1nc(N[C@H]2CC[C@]3(CCO3)CC2)nn2ccc(-c3ccc4ncccc4n3)c12] 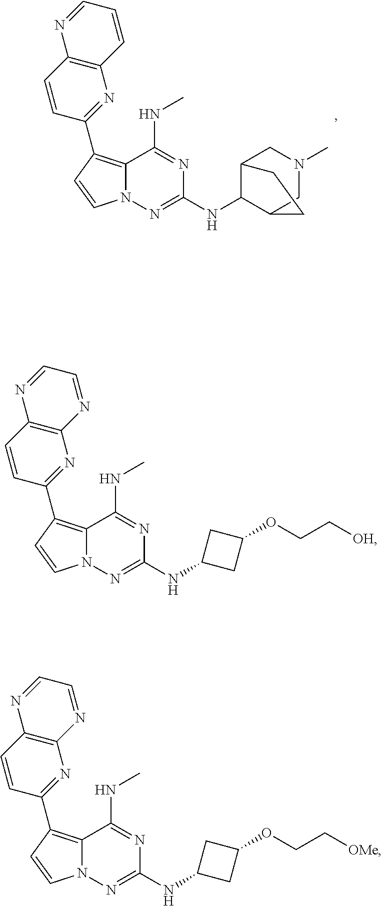 [SMILES: CNc1nc(NC2C3CCC2CN(C)C3)nn2ccc(-c3ccc4ncccc4n3)c12 ; CNc1nc(N[C@H]2C[C@@H](OCCO)C2)nn2ccc(-c3ccc4nccnc4n3)c12 ; CNc1nc(N[C@H]2C[C@@H](OCCOC)C2)nn2ccc(-c3ccc4nccnc4n3)c12] 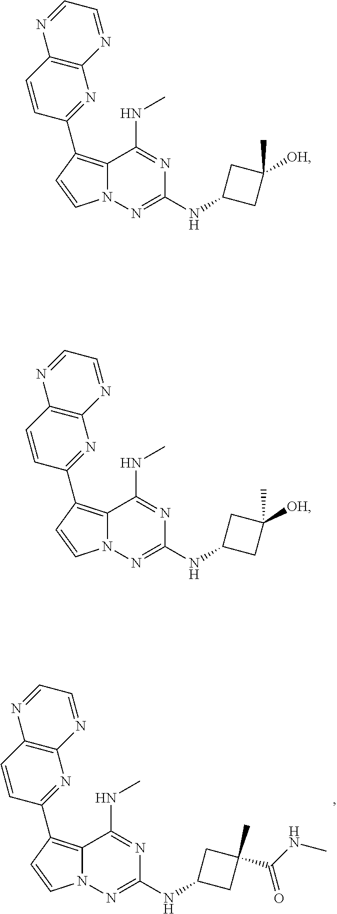 [SMILES: CNc1nc(N[C@H]2C[C@@](C)(O)C2)nn2ccc(-c3ccc4nccnc4n3)c12 ; CNc1nc(N[C@H]2C[C@](C)(O)C2)nn2ccc(-c3ccc4nccnc4n3)c12 ; CNc1nc(N[C@H]2C[C@@](C)(C(=O)NC)C2)nn2ccc(-c3ccc4nccnc4n3)c12] 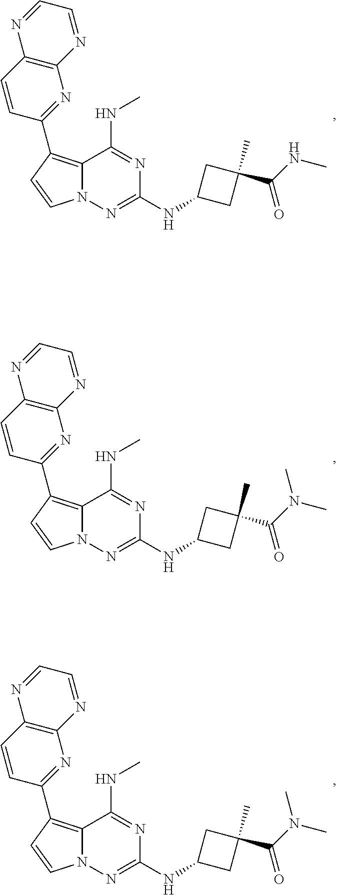 [SMILES: CNc1nc(N[C@H]2C[C@](C)(C(=O)NC)C2)nn2ccc(-c3ccc4nccnc4n3)c12 ; CNc1nc(N[C@H]2C[C@@](C)(C(=O)N(C)C)C2)nn2ccc(-c3ccc4nccnc4n3)c12 ; CNc1nc(N[C@H]2C[C@](C)(C(=O)N(C)C)C2)nn2ccc(-c3ccc4nccnc4n3)c12] 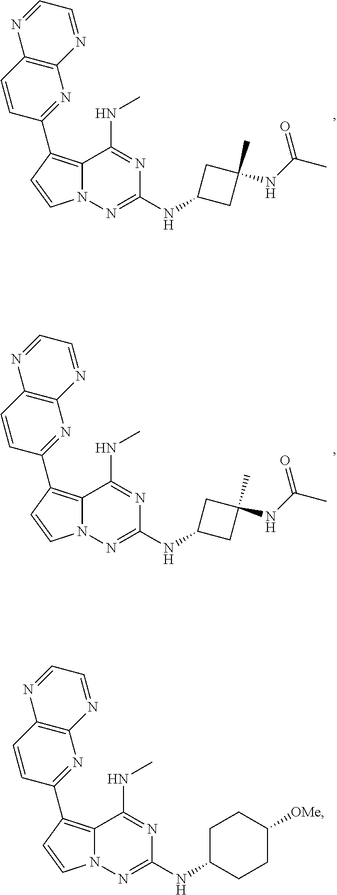 [SMILES: CNc1nc(N[C@H]2C[C@@](C)(NC(C)=O)C2)nn2ccc(-c3ccc4nccnc4n3)c12 ; CNc1nc(N[C@H]2C[C@](C)(NC(C)=O)C2)nn2ccc(-c3ccc4nccnc4n3)c12 ; CNc1nc(N[C@H]2CC[C@@H](OC)CC2)nn2ccc(-c3ccc4nccnc4n3)c12] 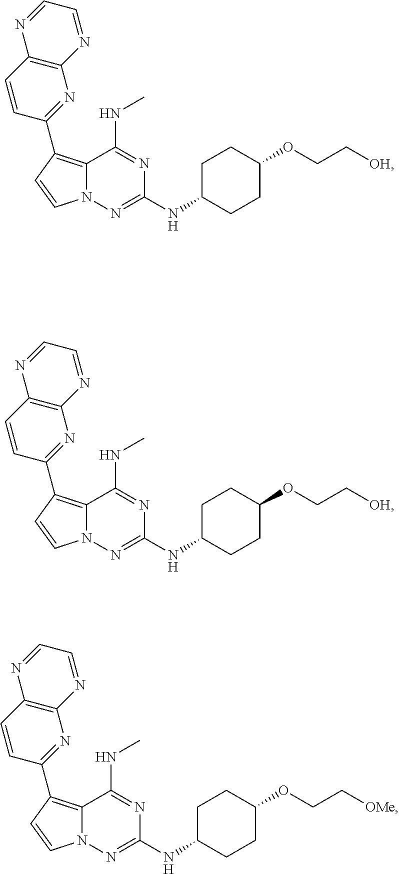 [SMILES: CNc1nc(N[C@H]2CC[C@@H](OCCO)CC2)nn2ccc(-c3ccc4nccnc4n3)c12 ; CNc1nc(N[C@H]2CC[C@H](OCCO)CC2)nn2ccc(-c3ccc4nccnc4n3)c12 ; CNc1nc(N[C@H]2CC[C@@H](OCCOC)CC2)nn2ccc(-c3ccc4nccnc4n3)c12] 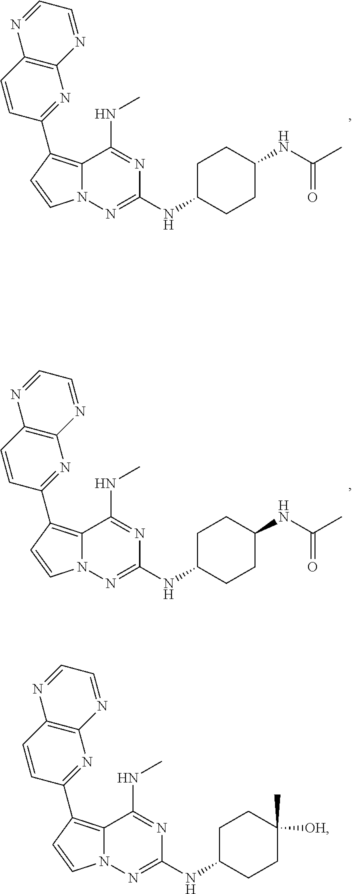 [SMILES: CNc1nc(N[C@H]2CC[C@@H](NC(C)=O)CC2)nn2ccc(-c3ccc4nccnc4n3)c12 ; CNc1nc(N[C@H]2CC[C@H](NC(C)=O)CC2)nn2ccc(-c3ccc4nccnc4n3)c12 ; CNc1nc(N[C@H]2CC[C@@](C)(O)CC2)nn2ccc(-c3ccc4nccnc4n3)c12] 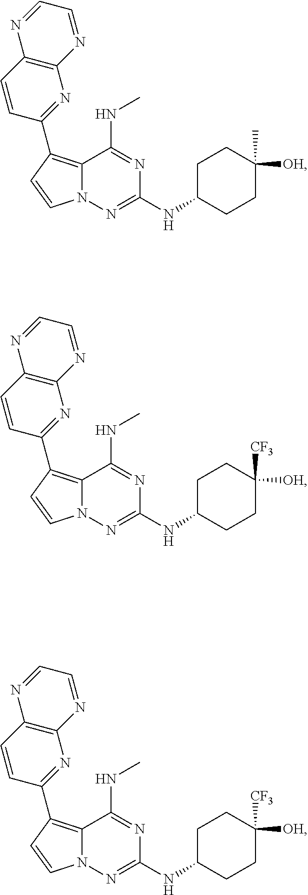 [SMILES: CNc1nc(N[C@H]2CC[C@](C)(O)CC2)nn2ccc(-c3ccc4nccnc4n3)c12 ; CNc1nc(N[C@H]2CC[C@](O)(C(F)(F)F)CC2)nn2ccc(-c3ccc4nccnc4n3)c12 ; CNc1nc(N[C@H]2CC[C@@](O)(C(F)(F)F)CC2)nn2ccc(-c3ccc4nccnc4n3)c12] 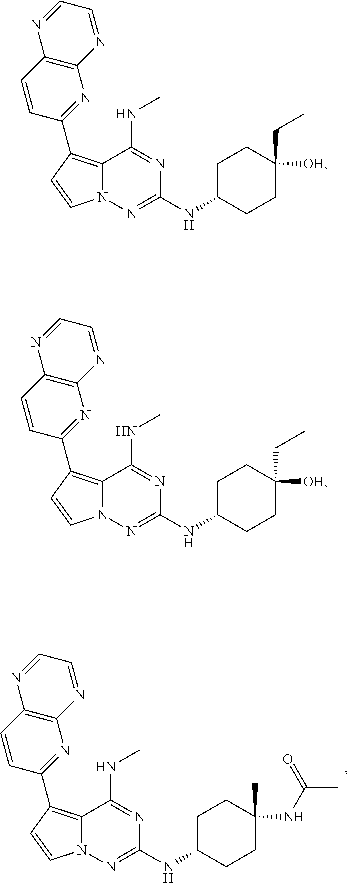 [SMILES: CC[C@]1(O)CC[C@@H](Nc2nc(NC)c3c(-c4ccc5nccnc5n4)ccn3n2)CC1 ; CC[C@]1(O)CC[C@H](Nc2nc(NC)c3c(-c4ccc5nccnc5n4)ccn3n2)CC1 ; CNc1nc(N[C@H]2CC[C@@](C)(NC(C)=O)CC2)nn2ccc(-c3ccc4nccnc4n3)c12] 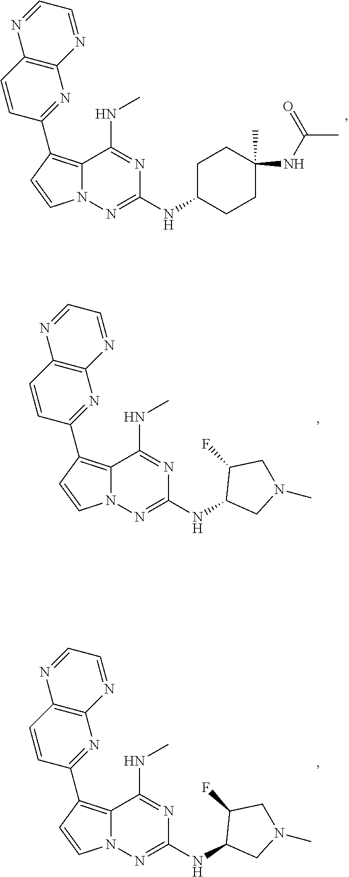 [SMILES: CNc1nc(N[C@H]2CC[C@](C)(NC(C)=O)CC2)nn2ccc(-c3ccc4nccnc4n3)c12 ; CNc1nc(N[C@H]2CN(C)C[C@H]2F)nn2ccc(-c3ccc4nccnc4n3)c12 ; CNc1nc(N[C@@H]2CN(C)C[C@@H]2F)nn2ccc(-c3ccc4nccnc4n3)c12] 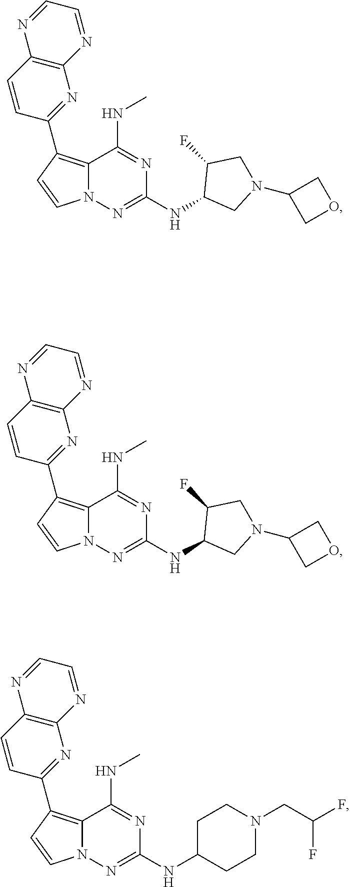 [SMILES: CNc1nc(N[C@H]2CN(C3COC3)C[C@H]2F)nn2ccc(-c3ccc4nccnc4n3)c12 ; CNc1nc(N[C@@H]2CN(C3COC3)C[C@@H]2F)nn2ccc(-c3ccc4nccnc4n3)c12 ; CNc1nc(NC2CCN(CC(F)F)CC2)nn2ccc(-c3ccc4nccnc4n3)c12] 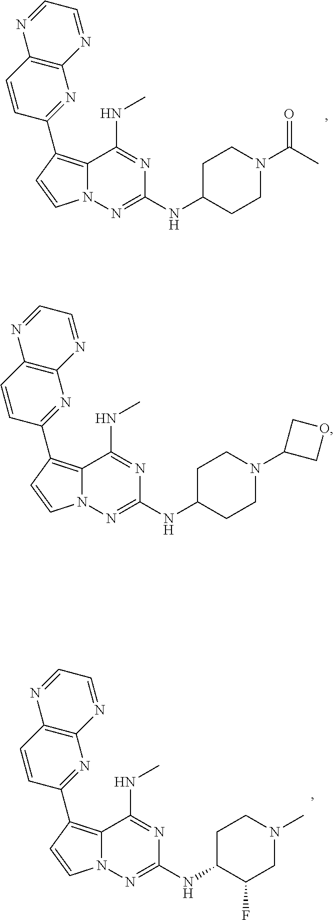 [SMILES: CNc1nc(NC2CCN(C(C)=O)CC2)nn2ccc(-c3ccc4nccnc4n3)c12 ; CNc1nc(NC2CCN(C3COC3)CC2)nn2ccc(-c3ccc4nccnc4n3)c12 ; CNc1nc(N[C@@H]2CCN(C)C[C@@H]2F)nn2ccc(-c3ccc4nccnc4n3)c12] 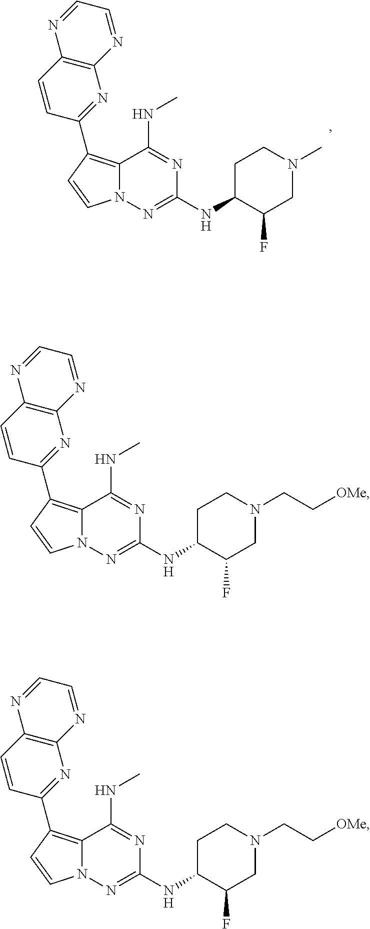 [SMILES: CNc1nc(N[C@H]2CCN(C)C[C@H]2F)nn2ccc(-c3ccc4nccnc4n3)c12 ; CNc1nc(N[C@@H]2CCN(CCOC)C[C@@H]2F)nn2ccc(-c3ccc4nccnc4n3)c12 ; CNc1nc(N[C@@H]2CCN(CCOC)C[C@H]2F)nn2ccc(-c3ccc4nccnc4n3)c12] 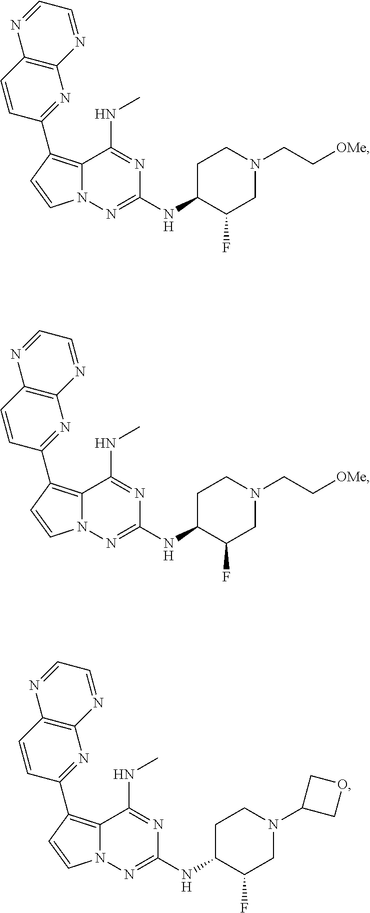 [SMILES: CNc1nc(N[C@H]2CCN(CCOC)C[C@@H]2F)nn2ccc(-c3ccc4nccnc4n3)c12 ; CNc1nc(N[C@H]2CCN(CCOC)C[C@H]2F)nn2ccc(-c3ccc4nccnc4n3)c12 ; CNc1nc(N[C@@H]2CCN(C3COC3)C[C@@H]2F)nn2ccc(-c3ccc4nccnc4n3)c12] 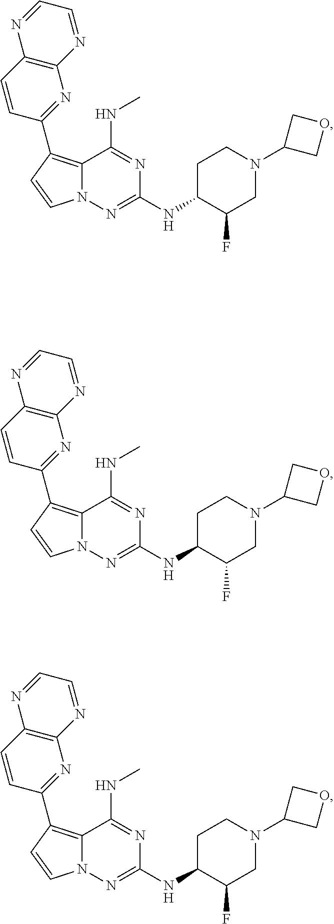 [SMILES: CNc1nc(N[C@@H]2CCN(C3COC3)C[C@H]2F)nn2ccc(-c3ccc4nccnc4n3)c12 ; CNc1nc(N[C@H]2CCN(C3COC3)C[C@@H]2F)nn2ccc(-c3ccc4nccnc4n3)c12 ; CNc1nc(N[C@H]2CCN(C3COC3)C[C@H]2F)nn2ccc(-c3ccc4nccnc4n3)c12] 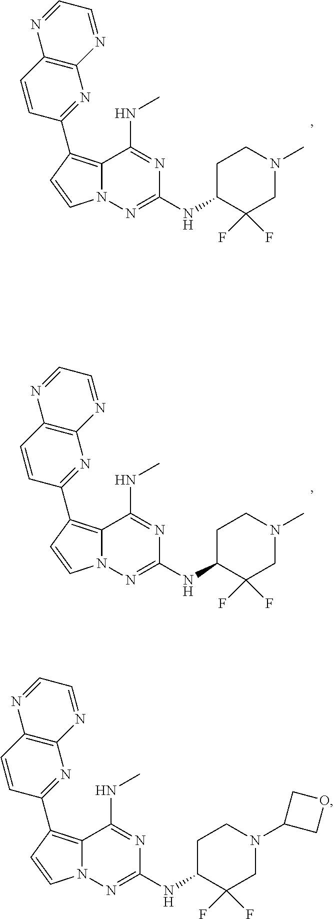 [SMILES: CNc1nc(N[C@@H]2CCN(C)CC2(F)F)nn2ccc(-c3ccc4nccnc4n3)c12 ; CNc1nc(N[C@H]2CCN(C)CC2(F)F)nn2ccc(-c3ccc4nccnc4n3)c12 ; CNc1nc(N[C@@H]2CCN(C3COC3)CC2(F)F)nn2ccc(-c3ccc4nccnc4n3)c12] 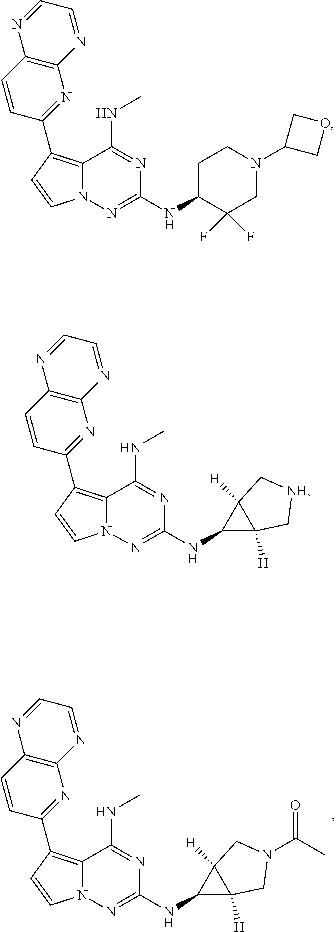 [SMILES: CNc1nc(N[C@H]2CCN(C3COC3)CC2(F)F)nn2ccc(-c3ccc4nccnc4n3)c12 ; CNc1nc(N[C@@H]2[C@@H]3CNC[C@@H]32)nn2ccc(-c3ccc4nccnc4n3)c12 ; CNc1nc(N[C@@H]2[C@@H]3CN(C(C)=O)C[C@@H]32)nn2ccc(-c3ccc4nccnc4n3)c12] 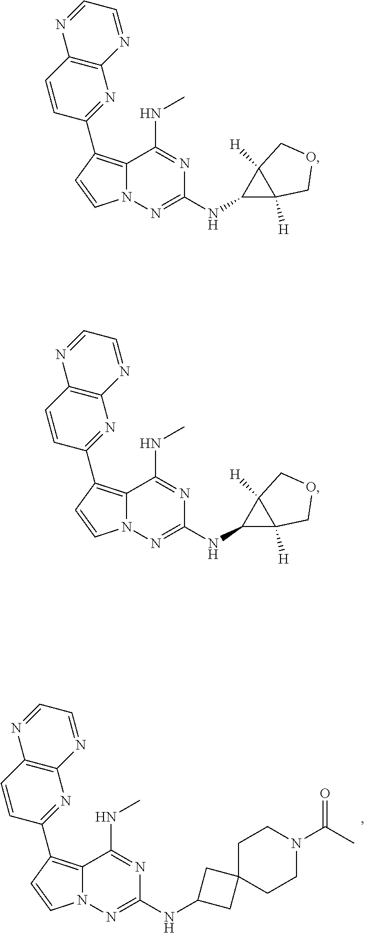 [SMILES: CNc1nc(N[C@H]2[C@@H]3COC[C@@H]32)nn2ccc(-c3ccc4nccnc4n3)c12 ; CNc1nc(N[C@@H]2[C@@H]3COC[C@@H]32)nn2ccc(-c3ccc4nccnc4n3)c12 ; CNc1nc(NC2CC3(CCN(C(C)=O)CC3)C2)nn2ccc(-c3ccc4nccnc4n3)c12] 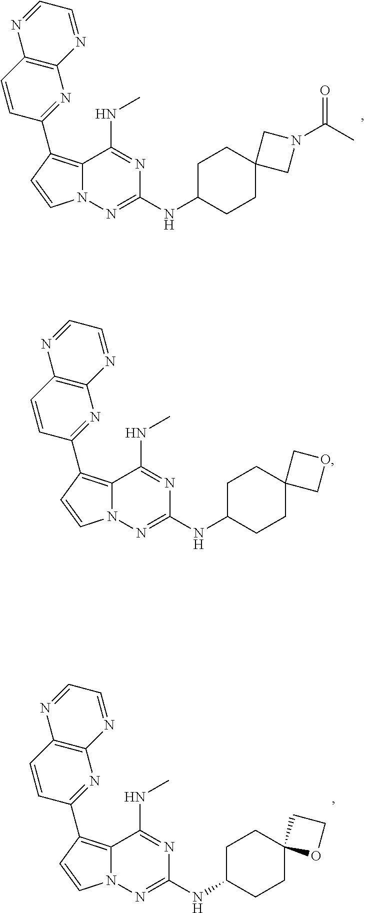 [SMILES: CNc1nc(NC2CCC3(CC2)CN(C(C)=O)C3)nn2ccc(-c3ccc4nccnc4n3)c12 ; CNc1nc(NC2CCC3(CC2)COC3)nn2ccc(-c3ccc4nccnc4n3)c12 ; CNc1nc(N[C@H]2CC[C@@]3(CCO3)CC2)nn2ccc(-c3ccc4nccnc4n3)c12] 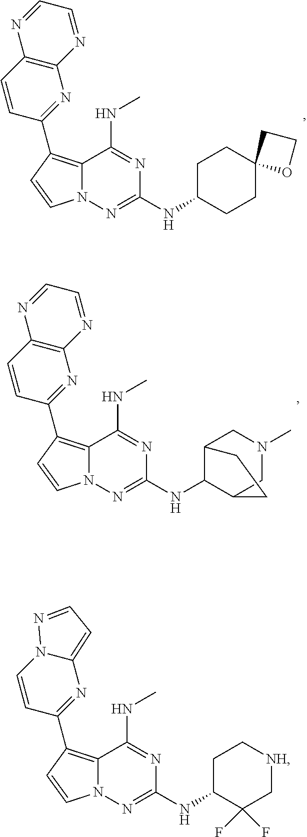 [SMILES: CNc1nc(N[C@H]2CC[C@]3(CCO3)CC2)nn2ccc(-c3ccc4nccnc4n3)c12 ; CNc1nc(NC2C3CCC2CN(C)C3)nn2ccc(-c3ccc4nccnc4n3)c12 ; CNc1nc(N[C@@H]2CCNCC2(F)F)nn2ccc(-c3ccn4nccc4n3)c12] 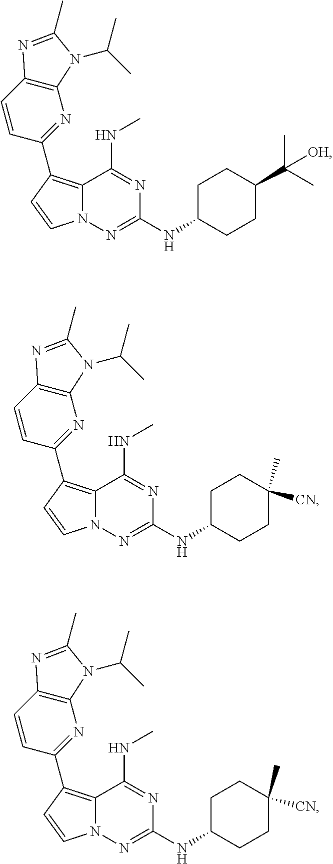 [SMILES: CNc1nc(N[C@H]2CC[C@H](C(C)(C)O)CC2)nn2ccc(-c3ccc4nc(C)n(C(C)C)c4n3)c12 ; CNc1nc(N[C@H]2CC[C@](C)(C#N)CC2)nn2ccc(-c3ccc4nc(C)n(C(C)C)c4n3)c12 ; CNc1nc(N[C@H]2CC[C@@](C)(C#N)CC2)nn2ccc(-c3ccc4nc(C)n(C(C)C)c4n3)c12] 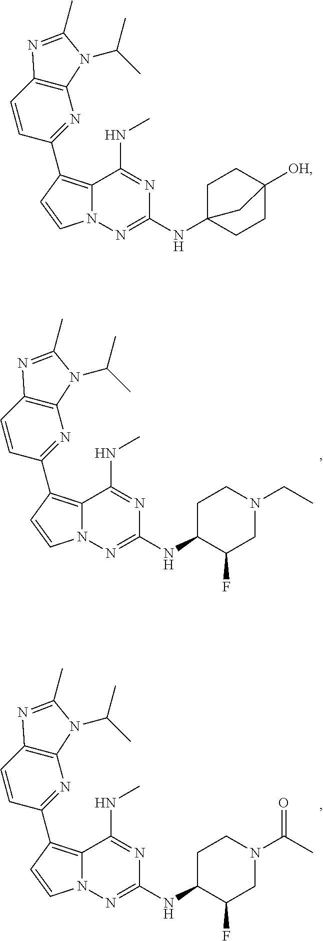 [SMILES: CNc1nc(NC23CCC(O)(CC2)C3)nn2ccc(-c3ccc4nc(C)n(C(C)C)c4n3)c12 ; CCN1CC[C@H](Nc2nc(NC)c3c(-c4ccc5nc(C)n(C(C)C)c5n4)ccn3n2)[C@H](F)C1 ; CNc1nc(N[C@H]2CCN(C(C)=O)C[C@H]2F)nn2ccc(-c3ccc4nc(C)n(C(C)C)c4n3)c12] 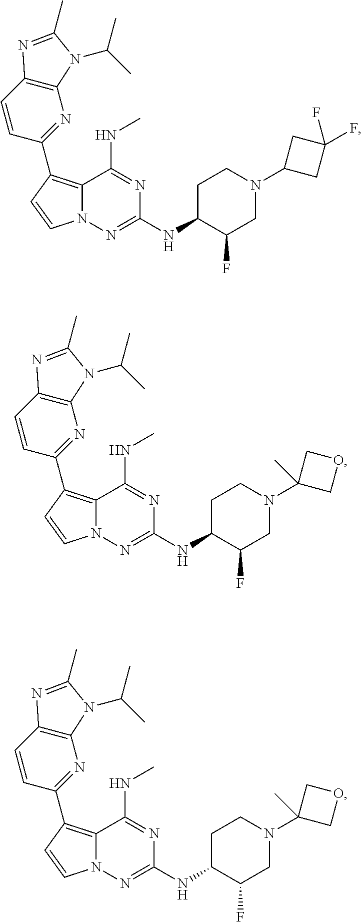 [SMILES: CNc1nc(N[C@H]2CCN(C3CC(F)(F)C3)C[C@H]2F)nn2ccc(-c3ccc4nc(C)n(C(C)C)c4n3)c12 ; CNc1nc(N[C@H]2CCN(C3(C)COC3)C[C@H]2F)nn2ccc(-c3ccc4nc(C)n(C(C)C)c4n3)c12 ; CNc1nc(N[C@@H]2CCN(C3(C)COC3)C[C@@H]2F)nn2ccc(-c3ccc4nc(C)n(C(C)C)c4n3)c12] 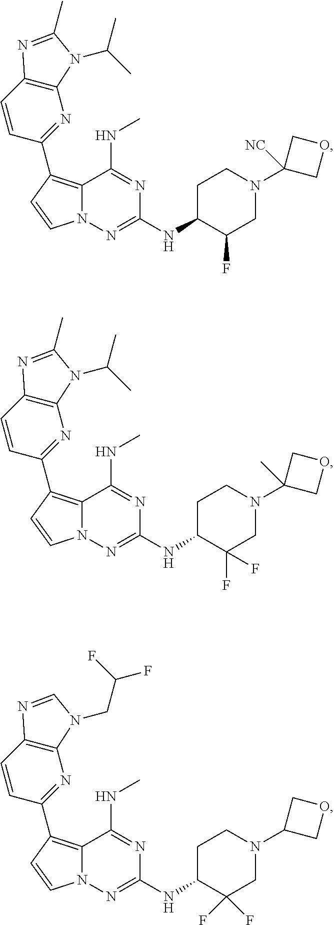 [SMILES: CNc1nc(N[C@H]2CCN(C3(C#N)COC3)C[C@H]2F)nn2ccc(-c3ccc4nc(C)n(C(C)C)c4n3)c12 ; CNc1nc(N[C@@H]2CCN(C3(C)COC3)CC2(F)F)nn2ccc(-c3ccc4nc(C)n(C(C)C)c4n3)c12 ; CNc1nc(N[C@@H]2CCN(C3COC3)CC2(F)F)nn2ccc(-c3ccc4ncn(CC(F)F)c4n3)c12] 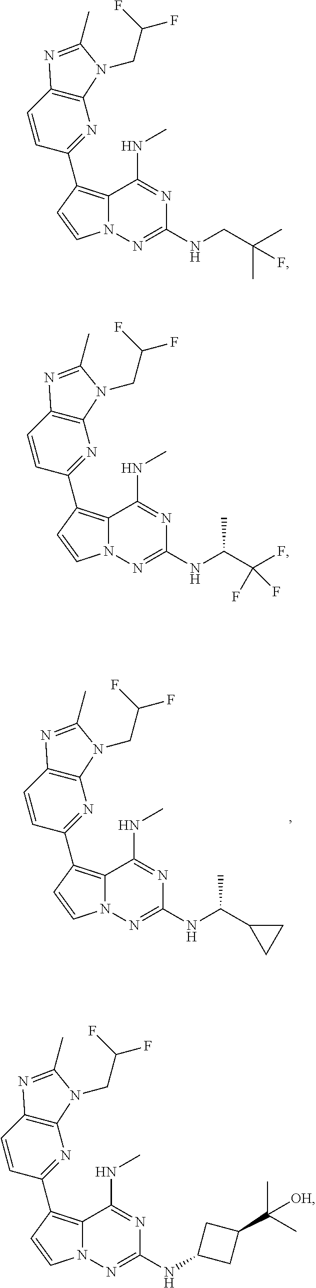 [SMILES: CNc1nc(NCC(C)(C)F)nn2ccc(-c3ccc4nc(C)n(CC(F)F)c4n3)c12 ; CNc1nc(N[C@H](C)C(F)(F)F)nn2ccc(-c3ccc4nc(C)n(CC(F)F)c4n3)c12 ; CNc1nc(N[C@H](C)C2CC2)nn2ccc(-c3ccc4nc(C)n(CC(F)F)c4n3)c12 ; CNc1nc(N[C@H]2C[C@H](C(C)(C)O)C2)nn2ccc(-c3ccc4nc(C)n(CC(F)F)c4n3)c12] 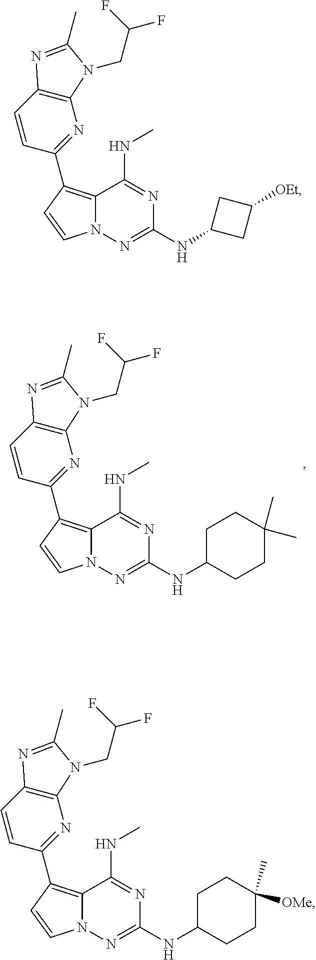 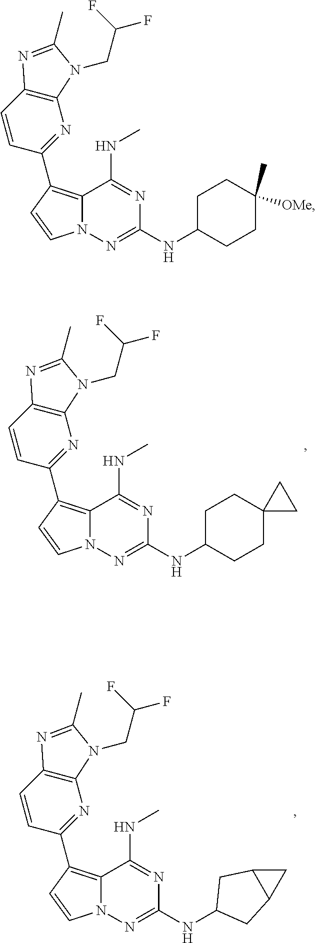 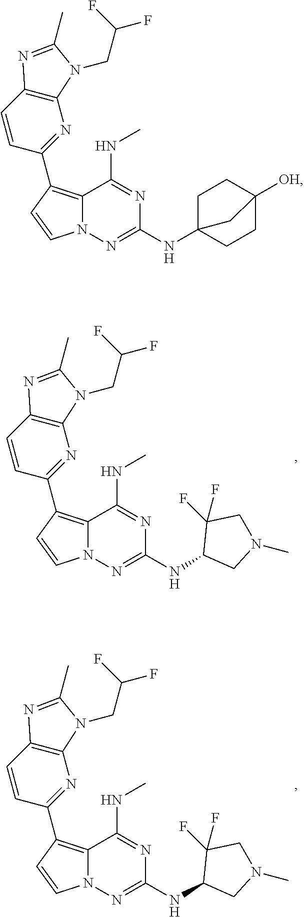 [SMILES: CNc1nc(NC23CCC(O)(CC2)C3)nn2ccc(-c3ccc4nc(C)n(CC(F)F)c4n3)c12 ; CNc1nc(N[C@H]2CN(C)CC2(F)F)nn2ccc(-c3ccc4nc(C)n(CC(F)F)c4n3)c12 ; CNc1nc(N[C@@H]2CN(C)CC2(F)F)nn2ccc(-c3ccc4nc(C)n(CC(F)F)c4n3)c12] 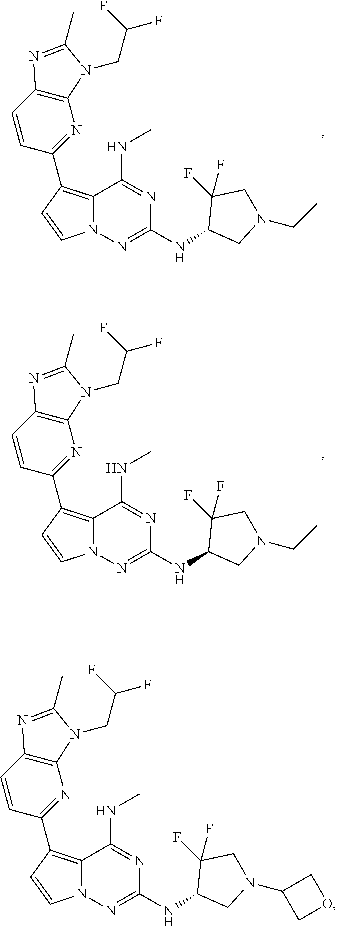 [SMILES: CCN1C[C@H](Nc2nc(NC)c3c(-c4ccc5nc(C)n(CC(F)F)c5n4)ccn3n2)C(F)(F)C1 ; CCN1C[C@@H](Nc2nc(NC)c3c(-c4ccc5nc(C)n(CC(F)F)c5n4)ccn3n2)C(F)(F)C1 ; CNc1nc(N[C@H]2CN(C3COC3)CC2(F)F)nn2ccc(-c3ccc4nc(C)n(CC(F)F)c4n3)c12] 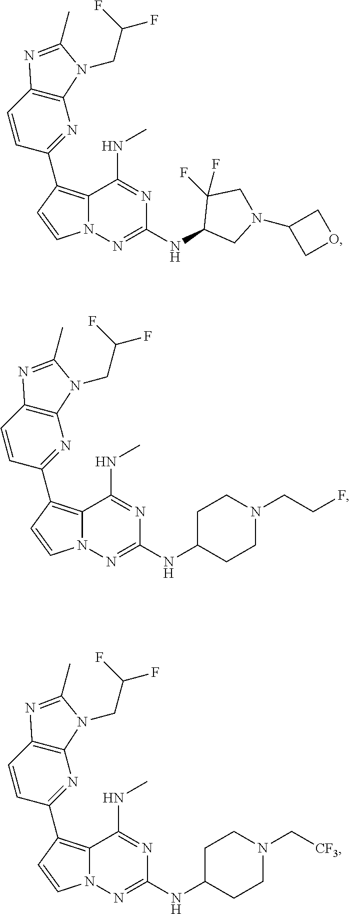 [SMILES: CNc1nc(N[C@@H]2CN(C3COC3)CC2(F)F)nn2ccc(-c3ccc4nc(C)n(CC(F)F)c4n3)c12 ; CNc1nc(NC2CCN(CCF)CC2)nn2ccc(-c3ccc4nc(C)n(CC(F)F)c4n3)c12 ; CNc1nc(NC2CCN(CC(F)(F)F)CC2)nn2ccc(-c3ccc4nc(C)n(CC(F)F)c4n3)c12] 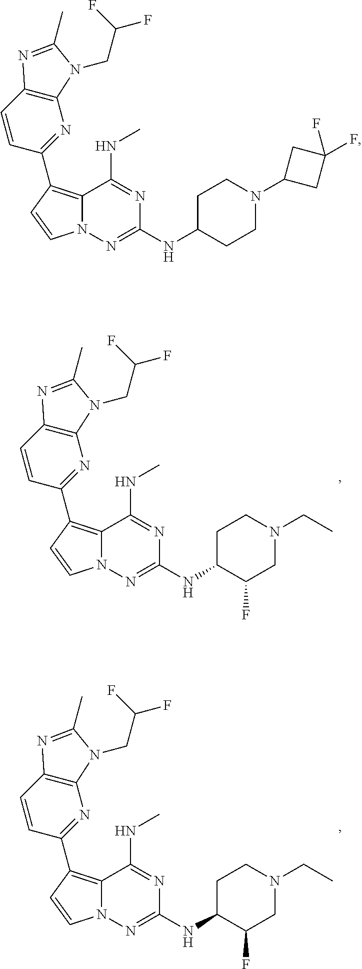 [SMILES: CNc1nc(NC2CCN(C3CC(F)(F)C3)CC2)nn2ccc(-c3ccc4nc(C)n(CC(F)F)c4n3)c12 ; CCN1CC[C@@H](Nc2nc(NC)c3c(-c4ccc5nc(C)n(CC(F)F)c5n4)ccn3n2)[C@@H](F)C1 ; CCN1CC[C@H](Nc2nc(NC)c3c(-c4ccc5nc(C)n(CC(F)F)c5n4)ccn3n2)[C@H](F)C1] 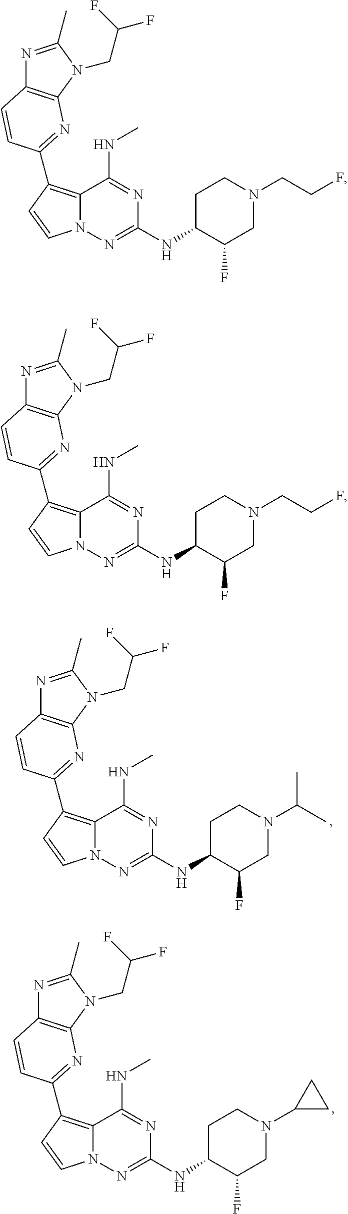 [SMILES: CNc1nc(N[C@@H]2CCN(CCF)C[C@@H]2F)nn2ccc(-c3ccc4nc(C)n(CC(F)F)c4n3)c12 ; CNc1nc(N[C@H]2CCN(CCF)C[C@H]2F)nn2ccc(-c3ccc4nc(C)n(CC(F)F)c4n3)c12 ; CNc1nc(N[C@H]2CCN(C(C)C)C[C@H]2F)nn2ccc(-c3ccc4nc(C)n(CC(F)F)c4n3)c12 ; CNc1nc(N[C@@H]2CCN(C3CC3)C[C@@H]2F)nn2ccc(-c3ccc4nc(C)n(CC(F)F)c4n3)c12] 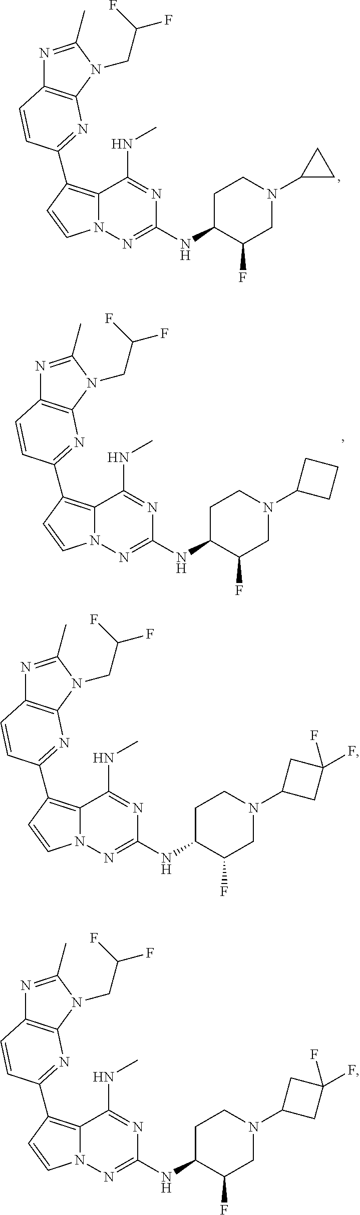 [SMILES: CNc1nc(N[C@H]2CCN(C3CC3)C[C@H]2F)nn2ccc(-c3ccc4nc(C)n(CC(F)F)c4n3)c12 ; CNc1nc(N[C@H]2CCN(C3CCC3)C[C@H]2F)nn2ccc(-c3ccc4nc(C)n(CC(F)F)c4n3)c12 ; CNc1nc(N[C@@H]2CCN(C3CC(F)(F)C3)C[C@@H]2F)nn2ccc(-c3ccc4nc(C)n(CC(F)F)c4n3)c12 ; CNc1nc(N[C@H]2CCN(C3CC(F)(F)C3)C[C@H]2F)nn2ccc(-c3ccc4nc(C)n(CC(F)F)c4n3)c12] 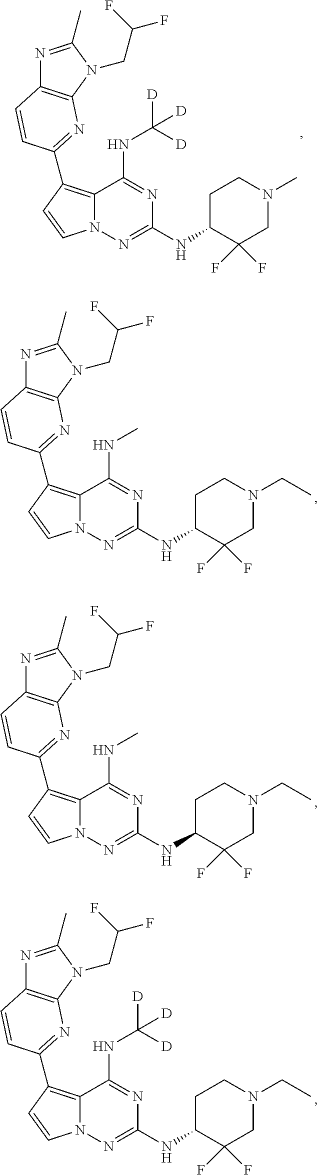 [SMILES: [2H]C([2H])([2H])Nc1nc(N[C@@H]2CCN(C)CC2(F)F)nn2ccc(-c3ccc4nc(C)n(CC(F)F)c4n3)c12 ; CCN1CC[C@@H](Nc2nc(NC)c3c(-c4ccc5nc(C)n(CC(F)F)c5n4)ccn3n2)C(F)(F)C1 ; CCN1CC[C@H](Nc2nc(NC)c3c(-c4ccc5nc(C)n(CC(F)F)c5n4)ccn3n2)C(F)(F)C1 ; [2H]C([2H])([2H])Nc1nc(N[C@@H]2CCN(CC)CC2(F)F)nn2ccc(-c3ccc4nc(C)n(CC(F)F)c4n3)c12] 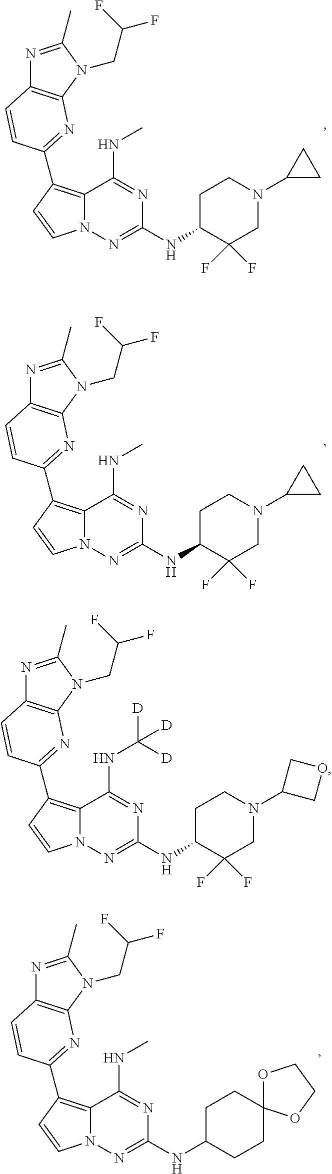 [SMILES: CNc1nc(N[C@@H]2CCN(C3CC3)CC2(F)F)nn2ccc(-c3ccc4nc(C)n(CC(F)F)c4n3)c12 ; CNc1nc(N[C@H]2CCN(C3CC3)CC2(F)F)nn2ccc(-c3ccc4nc(C)n(CC(F)F)c4n3)c12 ; [2H]C([2H])([2H])Nc1nc(N[C@@H]2CCN(C3COC3)CC2(F)F)nn2ccc(-c3ccc4nc(C)n(CC(F)F)c4n3)c12 ; CNc1nc(NC2CCC3(CC2)OCCO3)nn2ccc(-c3ccc4nc(C)n(CC(F)F)c4n3)c12] 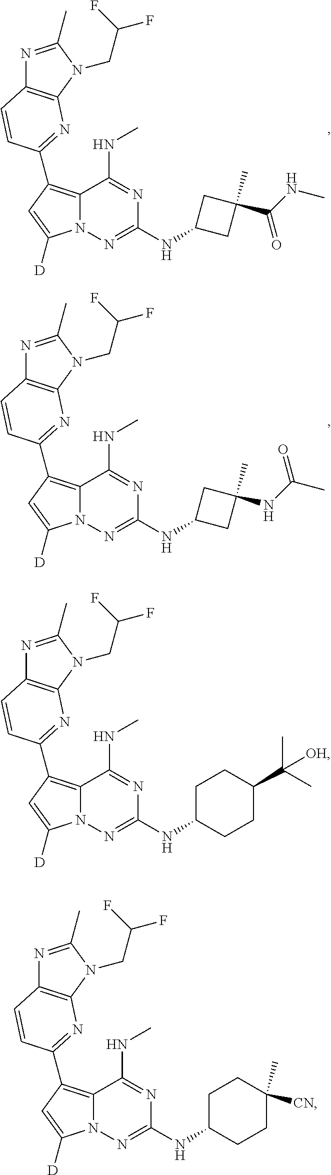 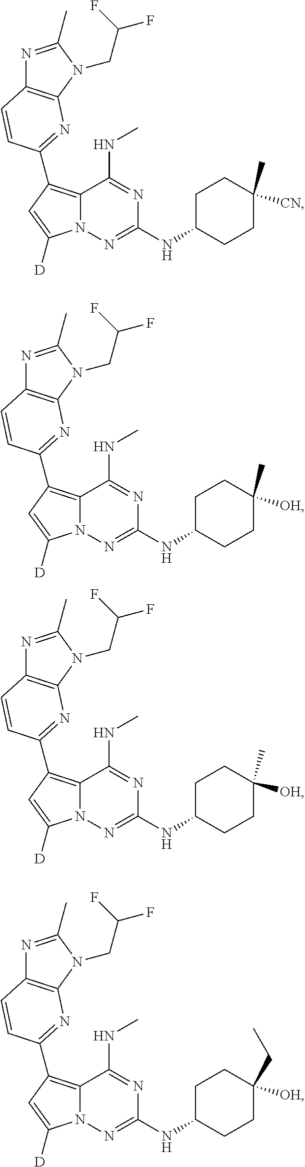 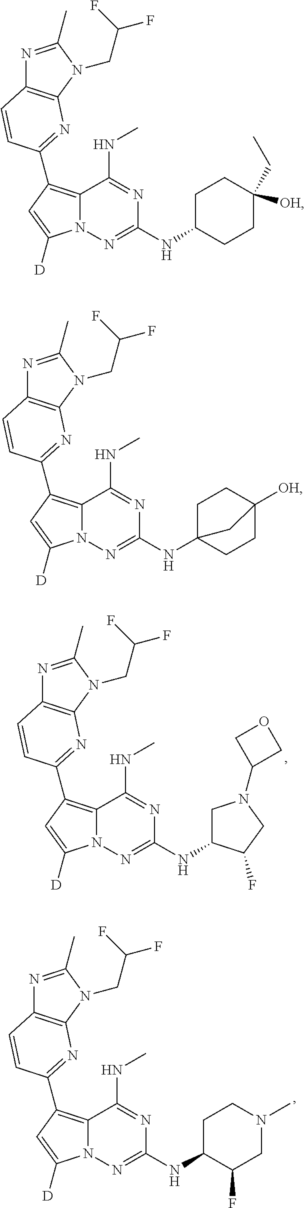 [SMILES: [2H]c1cc(-c2ccc3nc(C)n(CC(F)F)c3n2)c2c(NC)nc(N[C@H]3CC[C@@](O)(CC)CC3)nn12 ; [2H]c1cc(-c2ccc3nc(C)n(CC(F)F)c3n2)c2c(NC)nc(NC34CCC(O)(CC3)C4)nn12 ; [2H]c1cc(-c2ccc3nc(C)n(CC(F)F)c3n2)c2c(NC)nc(N[C@@H]3CN(C4COC4)C[C@@H]3F)nn12 ; [2H]c1cc(-c2ccc3nc(C)n(CC(F)F)c3n2)c2c(NC)nc(N[C@H]3CCN(C)C[C@H]3F)nn12] 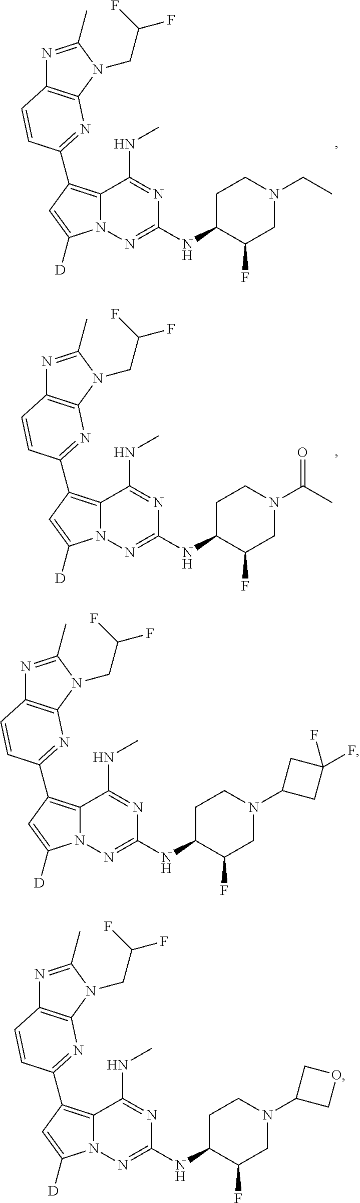 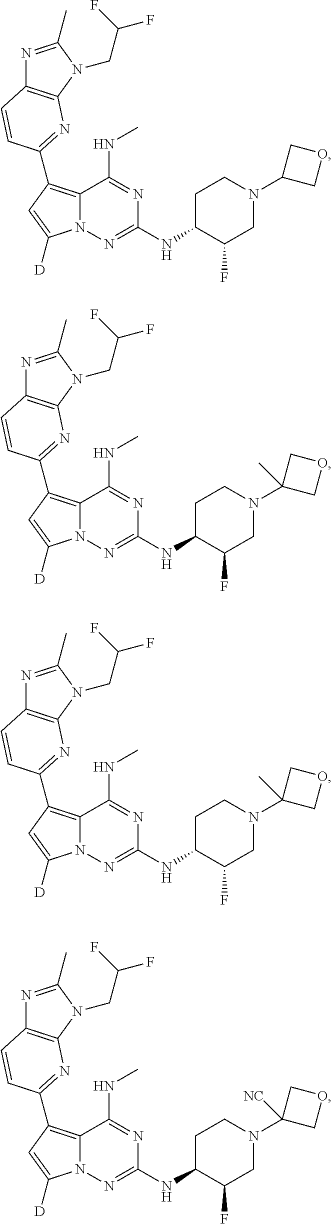 [SMILES: [2H]c1cc(-c2ccc3nc(C)n(CC(F)F)c3n2)c2c(NC)nc(N[C@@H]3CCN(C4COC4)C[C@@H]3F)nn12 ; [2H]c1cc(-c2ccc3nc(C)n(CC(F)F)c3n2)c2c(NC)nc(N[C@H]3CCN(C4(C)COC4)C[C@H]3F)nn12 ; [2H]c1cc(-c2ccc3nc(C)n(CC(F)F)c3n2)c2c(NC)nc(N[C@@H]3CCN(C4(C)COC4)C[C@@H]3F)nn12 ; [2H]c1cc(-c2ccc3nc(C)n(CC(F)F)c3n2)c2c(NC)nc(N[C@H]3CCN(C4(C#N)COC4)C[C@H]3F)nn12] 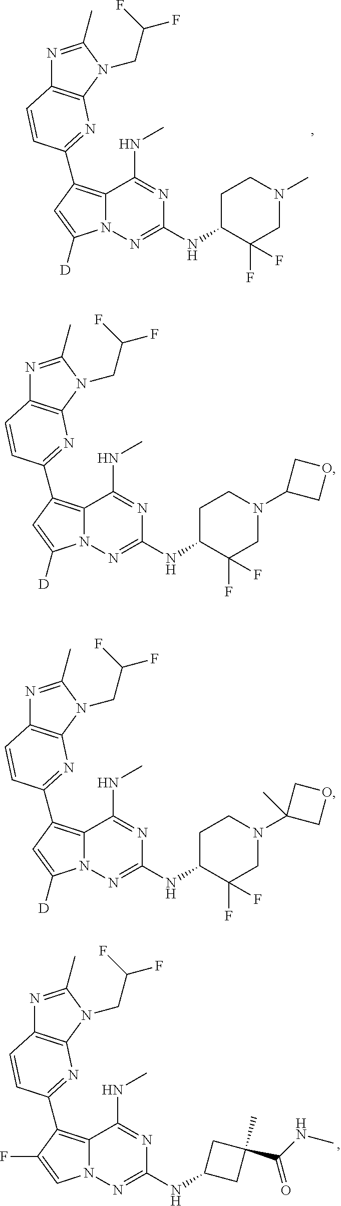 [SMILES: [2H]c1cc(-c2ccc3nc(C)n(CC(F)F)c3n2)c2c(NC)nc(N[C@@H]3CCN(C)CC3(F)F)nn12 ; [2H]c1cc(-c2ccc3nc(C)n(CC(F)F)c3n2)c2c(NC)nc(N[C@@H]3CCN(C4COC4)CC3(F)F)nn12 ; [2H]c1cc(-c2ccc3nc(C)n(CC(F)F)c3n2)c2c(NC)nc(N[C@@H]3CCN(C4(C)COC4)CC3(F)F)nn12 ; CNc1nc(N[C@H]2C[C@](C)(C(=O)NC)C2)nn2cc(F)c(-c3ccc4nc(C)n(CC(F)F)c4n3)c12] 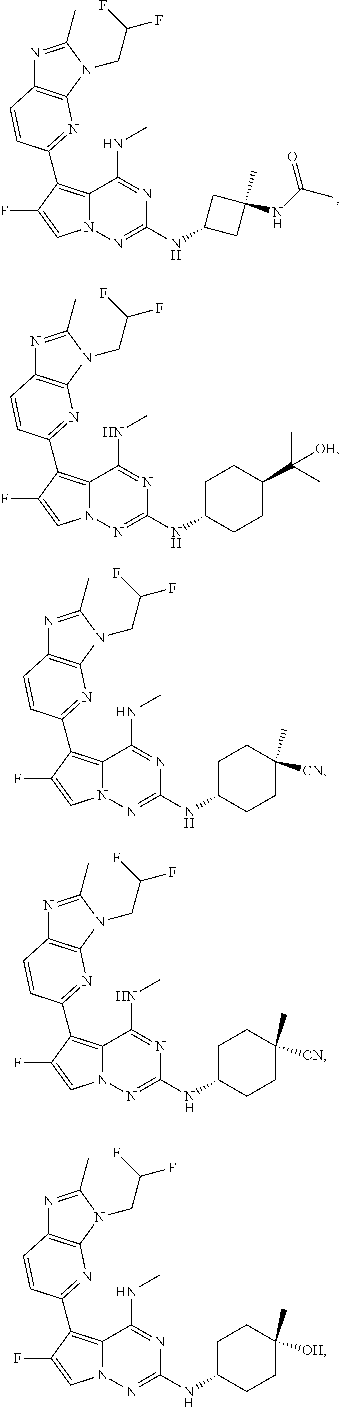 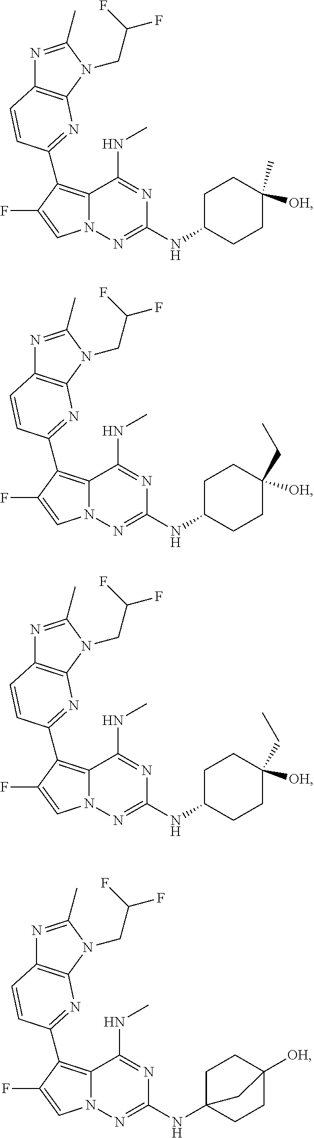 [SMILES: CNc1nc(N[C@H]2CC[C@](C)(O)CC2)nn2cc(F)c(-c3ccc4nc(C)n(CC(F)F)c4n3)c12 ; CC[C@]1(O)CC[C@@H](Nc2nc(NC)c3c(-c4ccc5nc(C)n(CC(F)F)c5n4)c(F)cn3n2)CC1 ; CC[C@]1(O)CC[C@H](Nc2nc(NC)c3c(-c4ccc5nc(C)n(CC(F)F)c5n4)c(F)cn3n2)CC1 ; CNc1nc(NC23CCC(O)(CC2)C3)nn2cc(F)c(-c3ccc4nc(C)n(CC(F)F)c4n3)c12] 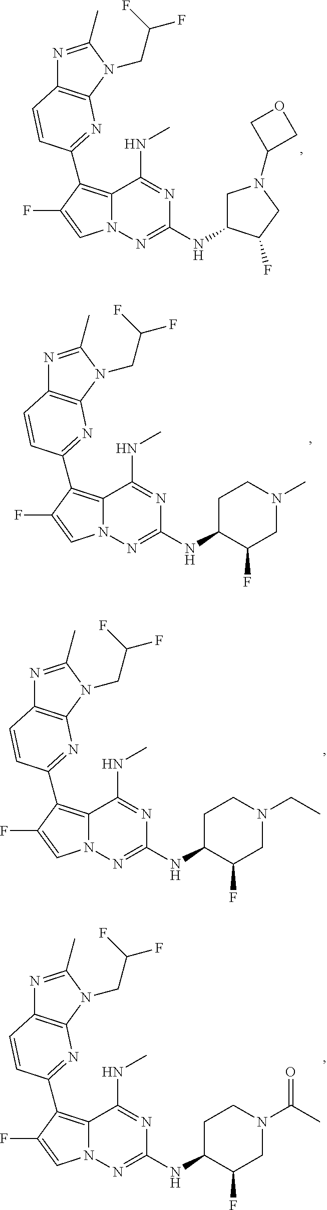 [SMILES: CNc1nc(N[C@@H]2CN(C3COC3)C[C@@H]2F)nn2cc(F)c(-c3ccc4nc(C)n(CC(F)F)c4n3)c12 ; CNc1nc(N[C@H]2CCN(C)C[C@H]2F)nn2cc(F)c(-c3ccc4nc(C)n(CC(F)F)c4n3)c12 ; CCN1CC[C@H](Nc2nc(NC)c3c(-c4ccc5nc(C)n(CC(F)F)c5n4)c(F)cn3n2)[C@H](F)C1 ; CNc1nc(N[C@H]2CCN(C(C)=O)C[C@H]2F)nn2cc(F)c(-c3ccc4nc(C)n(CC(F)F)c4n3)c12] 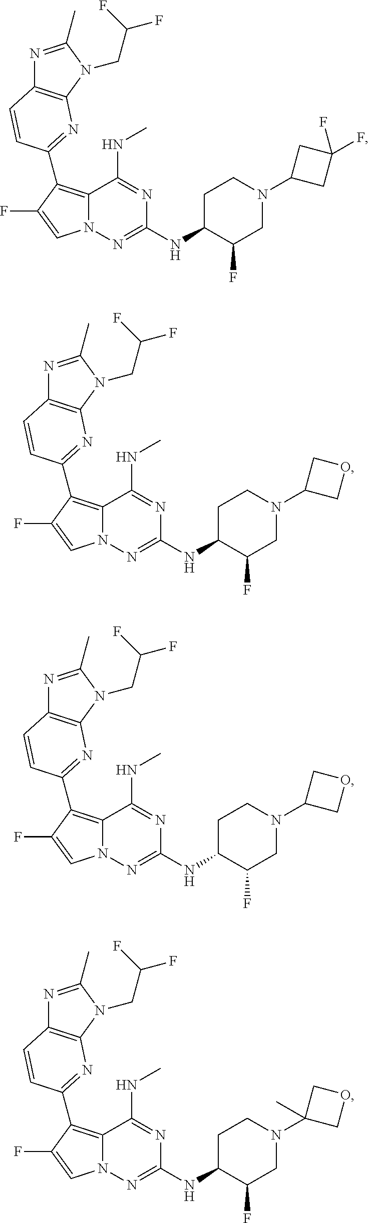 [SMILES: CNc1nc(N[C@H]2CCN(C3CC(F)(F)C3)C[C@H]2F)nn2cc(F)c(-c3ccc4nc(C)n(CC(F)F)c4n3)c12 ; CNc1nc(N[C@H]2CCN(C3COC3)C[C@H]2F)nn2cc(F)c(-c3ccc4nc(C)n(CC(F)F)c4n3)c12 ; CNc1nc(N[C@@H]2CCN(C3COC3)C[C@@H]2F)nn2cc(F)c(-c3ccc4nc(C)n(CC(F)F)c4n3)c12 ; CNc1nc(N[C@H]2CCN(C3(C)COC3)C[C@H]2F)nn2cc(F)c(-c3ccc4nc(C)n(CC(F)F)c4n3)c12] 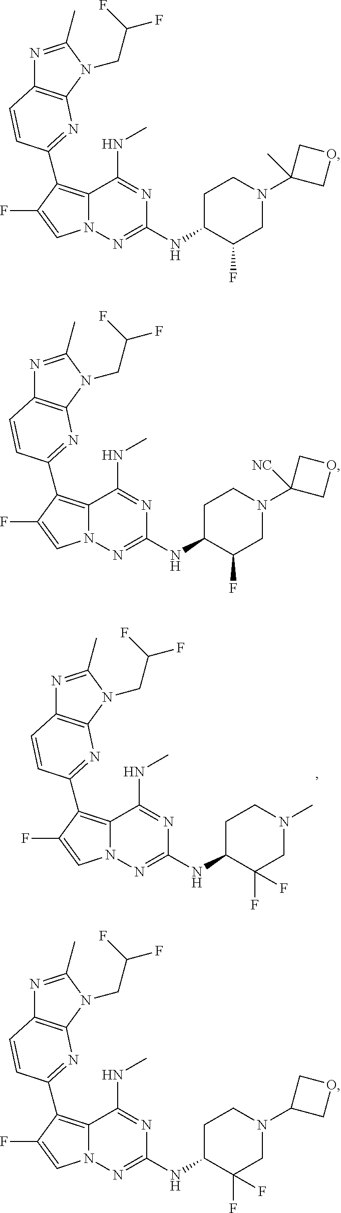 [SMILES: CNc1nc(N[C@@H]2CCN(C3(C)COC3)C[C@@H]2F)nn2cc(F)c(-c3ccc4nc(C)n(CC(F)F)c4n3)c12 ; CNc1nc(N[C@H]2CCN(C3(C#N)COC3)C[C@H]2F)nn2cc(F)c(-c3ccc4nc(C)n(CC(F)F)c4n3)c12 ; CNc1nc(N[C@H]2CCN(C)CC2(F)F)nn2cc(F)c(-c3ccc4nc(C)n(CC(F)F)c4n3)c12 ; CNc1nc(N[C@@H]2CCN(C3COC3)CC2(F)F)nn2cc(F)c(-c3ccc4nc(C)n(CC(F)F)c4n3)c12] 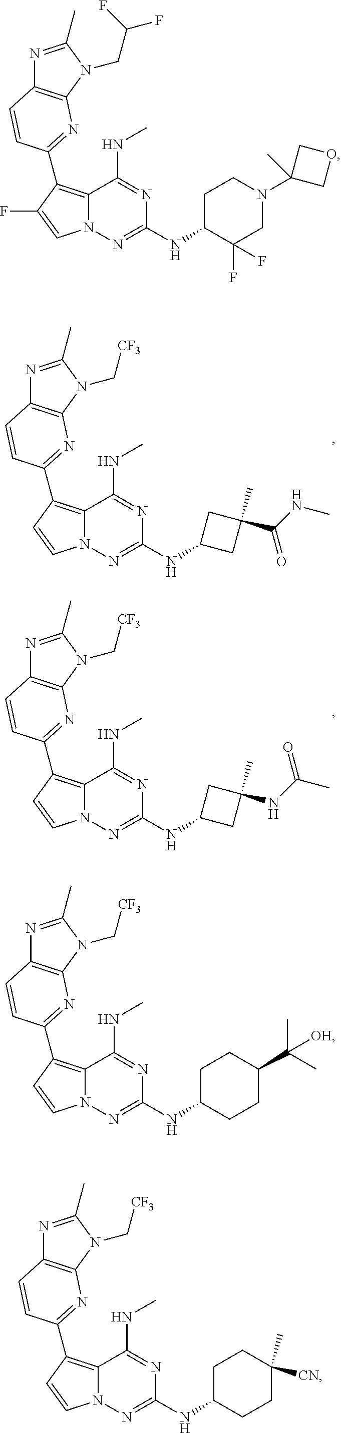 [SMILES: CNc1nc(N[C@@H]2CCN(C3(C)COC3)CC2(F)F)nn2cc(F)c(-c3ccc4nc(C)n(CC(F)F)c4n3)c12 ; CNc1nc(N[C@H]2C[C@](C)(C(=O)NC)C2)nn2ccc(-c3ccc4nc(C)n(CC(F)(F)F)c4n3)c12 ; CNc1nc(N[C@H]2C[C@](C)(NC(C)=O)C2)nn2ccc(-c3ccc4nc(C)n(CC(F)(F)F)c4n3)c12 ; CNc1nc(N[C@H]2CC[C@H](C(C)(C)O)CC2)nn2ccc(-c3ccc4nc(C)n(CC(F)(F)F)c4n3)c12 ; CNc1nc(N[C@H]2CC[C@](C)(C#N)CC2)nn2ccc(-c3ccc4nc(C)n(CC(F)(F)F)c4n3)c12] 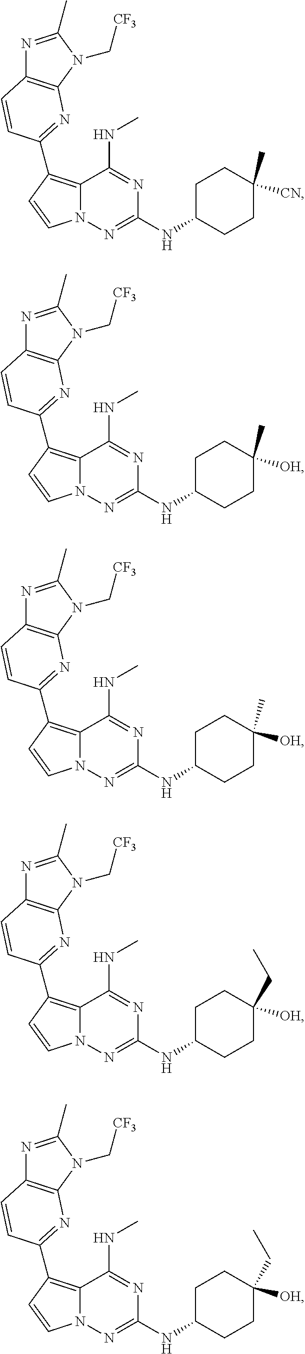 [SMILES: CNc1nc(N[C@H]2CC[C@@](C)(C#N)CC2)nn2ccc(-c3ccc4nc(C)n(CC(F)(F)F)c4n3)c12 ; CNc1nc(N[C@H]2CC[C@@](C)(O)CC2)nn2ccc(-c3ccc4nc(C)n(CC(F)(F)F)c4n3)c12 ; CNc1nc(N[C@H]2CC[C@](C)(O)CC2)nn2ccc(-c3ccc4nc(C)n(CC(F)(F)F)c4n3)c12 ; CC[C@]1(O)CC[C@@H](Nc2nc(NC)c3c(-c4ccc5nc(C)n(CC(F)(F)F)c5n4)ccn3n2)CC1 ; CC[C@]1(O)CC[C@H](Nc2nc(NC)c3c(-c4ccc5nc(C)n(CC(F)(F)F)c5n4)ccn3n2)CC1] 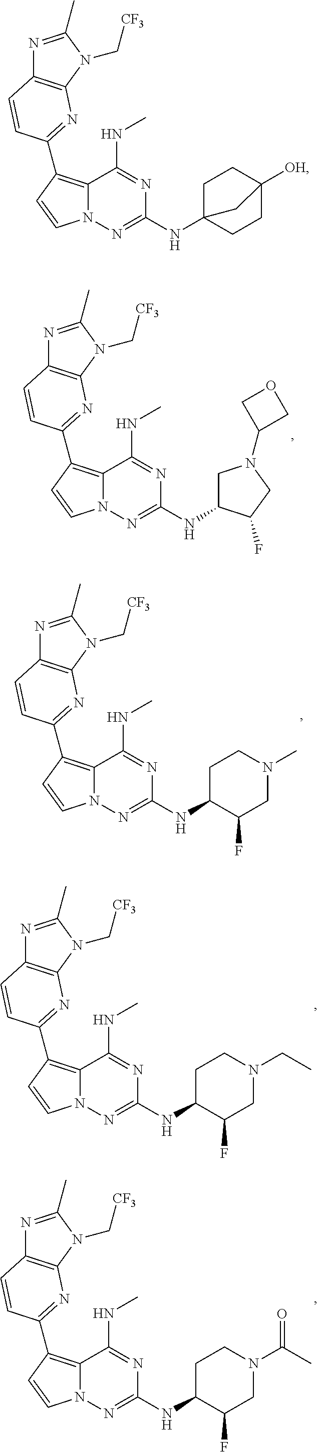 [SMILES: CNc1nc(NC23CCC(O)(CC2)C3)nn2ccc(-c3ccc4nc(C)n(CC(F)(F)F)c4n3)c12 ; CNc1nc(N[C@@H]2CN(C3COC3)C[C@@H]2F)nn2ccc(-c3ccc4nc(C)n(CC(F)(F)F)c4n3)c12 ; CNc1nc(N[C@H]2CCN(C)C[C@H]2F)nn2ccc(-c3ccc4nc(C)n(CC(F)(F)F)c4n3)c12 ; CCN1CC[C@H](Nc2nc(NC)c3c(-c4ccc5nc(C)n(CC(F)(F)F)c5n4)ccn3n2)[C@H](F)C1 ; CNc1nc(N[C@H]2CCN(C(C)=O)C[C@H]2F)nn2ccc(-c3ccc4nc(C)n(CC(F)(F)F)c4n3)c12] 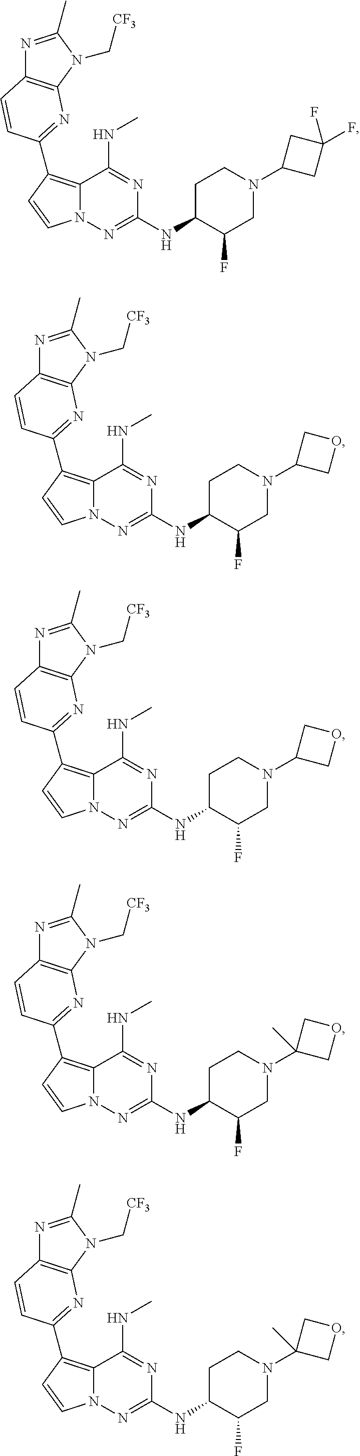 [SMILES: CNc1nc(N[C@H]2CCN(C3CC(F)(F)C3)C[C@H]2F)nn2ccc(-c3ccc4nc(C)n(CC(F)(F)F)c4n3)c12 ; CNc1nc(N[C@H]2CCN(C3COC3)C[C@H]2F)nn2ccc(-c3ccc4nc(C)n(CC(F)(F)F)c4n3)c12 ; CNc1nc(N[C@@H]2CCN(C3COC3)C[C@@H]2F)nn2ccc(-c3ccc4nc(C)n(CC(F)(F)F)c4n3)c12 ; CNc1nc(N[C@H]2CCN(C3(C)COC3)C[C@H]2F)nn2ccc(-c3ccc4nc(C)n(CC(F)(F)F)c4n3)c12 ; CNc1nc(N[C@@H]2CCN(C3(C)COC3)C[C@@H]2F)nn2ccc(-c3ccc4nc(C)n(CC(F)(F)F)c4n3)c12] 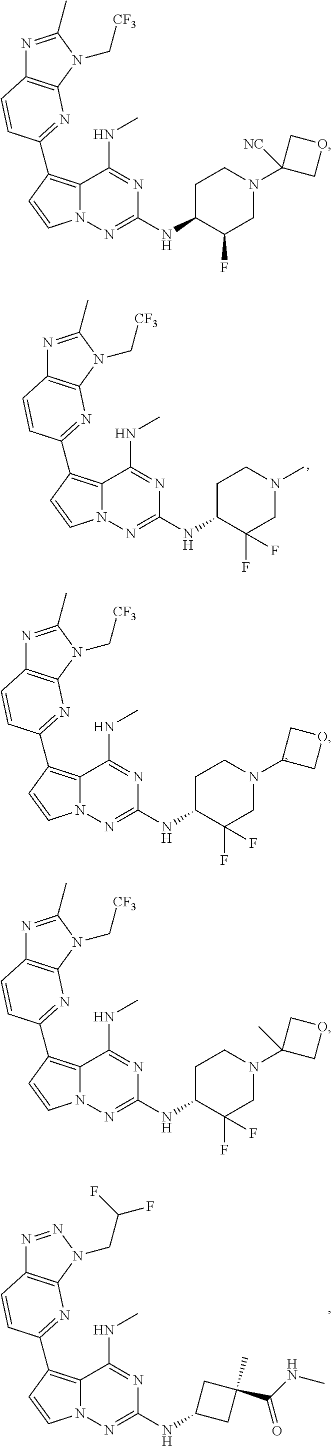 [SMILES: CNc1nc(N[C@H]2CCN(C3(C#N)COC3)C[C@H]2F)nn2ccc(-c3ccc4nc(C)n(CC(F)(F)F)c4n3)c12 ; CNc1nc(N[C@@H]2CCN(C)CC2(F)F)nn2ccc(-c3ccc4nc(C)n(CC(F)(F)F)c4n3)c12 ; CNc1nc(N[C@@H]2CCN(C3COC3)CC2(F)F)nn2ccc(-c3ccc4nc(C)n(CC(F)(F)F)c4n3)c12 ; CNc1nc(N[C@@H]2CCN(C3(C)COC3)CC2(F)F)nn2ccc(-c3ccc4nc(C)n(CC(F)(F)F)c4n3)c12 ; CNc1nc(N[C@H]2C[C@](C)(C(=O)NC)C2)nn2ccc(-c3ccc4nnn(CC(F)F)c4n3)c12] 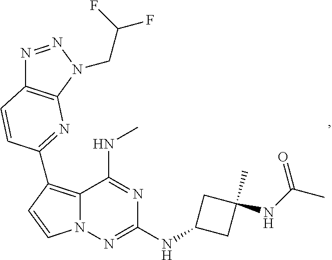 [SMILES: CNc1nc(N[C@H]2C[C@](C)(NC(C)=O)C2)nn2ccc(-c3ccc4nnn(CC(F)F)c4n3)c12] 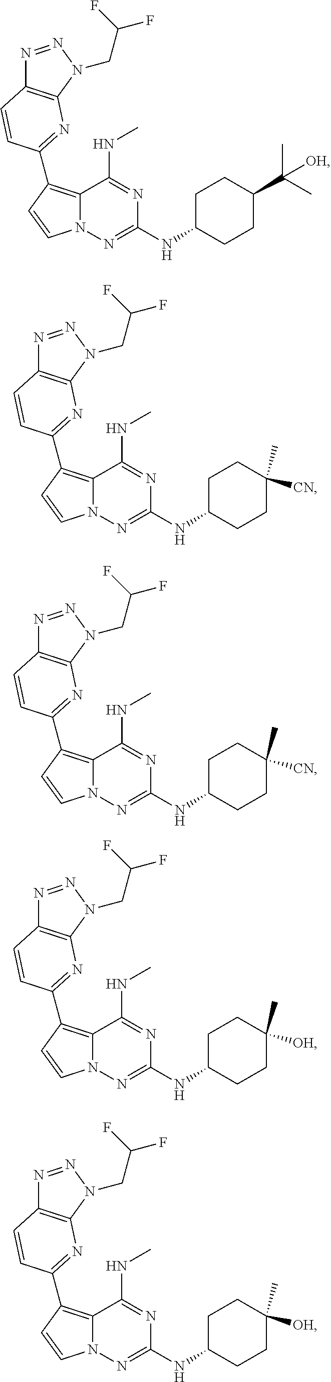 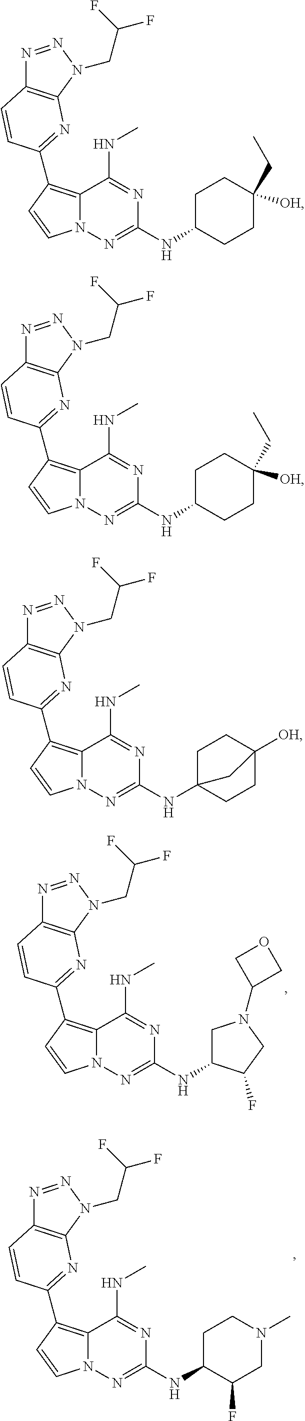 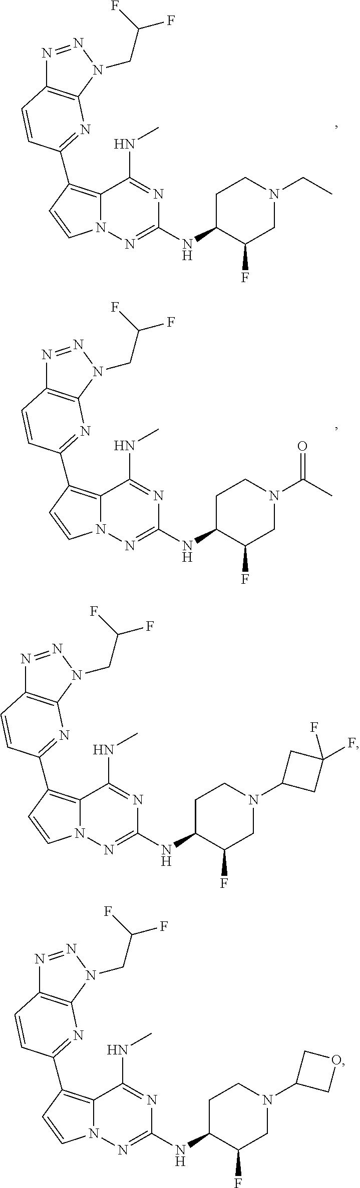 [SMILES: CCN1CC[C@H](Nc2nc(NC)c3c(-c4ccc5nnn(CC(F)F)c5n4)ccn3n2)[C@H](F)C1 ; CNc1nc(N[C@H]2CCN(C(C)=O)C[C@H]2F)nn2ccc(-c3ccc4nnn(CC(F)F)c4n3)c12 ; CNc1nc(N[C@H]2CCN(C3CC(F)(F)C3)C[C@H]2F)nn2ccc(-c3ccc4nnn(CC(F)F)c4n3)c12 ; CNc1nc(N[C@H]2CCN(C3COC3)C[C@H]2F)nn2ccc(-c3ccc4nnn(CC(F)F)c4n3)c12] 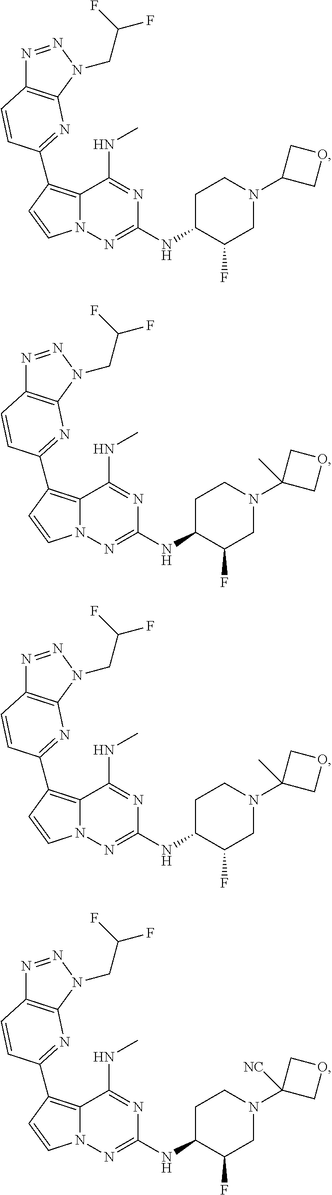 [SMILES: CNc1nc(N[C@@H]2CCN(C3COC3)C[C@@H]2F)nn2ccc(-c3ccc4nnn(CC(F)F)c4n3)c12 ; CNc1nc(N[C@H]2CCN(C3(C)COC3)C[C@H]2F)nn2ccc(-c3ccc4nnn(CC(F)F)c4n3)c12 ; CNc1nc(N[C@@H]2CCN(C3(C)COC3)C[C@@H]2F)nn2ccc(-c3ccc4nnn(CC(F)F)c4n3)c12 ; CNc1nc(N[C@H]2CCN(C3(C#N)COC3)C[C@H]2F)nn2ccc(-c3ccc4nnn(CC(F)F)c4n3)c12] 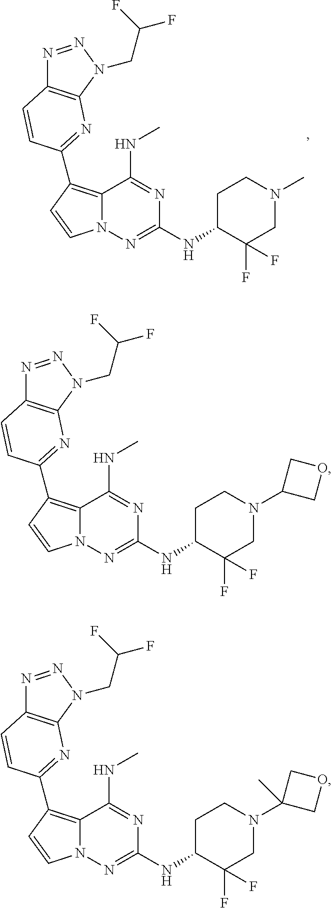 [SMILES: CNc1nc(N[C@@H]2CCN(C)CC2(F)F)nn2ccc(-c3ccc4nnn(CC(F)F)c4n3)c12 ; CNc1nc(N[C@@H]2CCN(C3COC3)CC2(F)F)nn2ccc(-c3ccc4nnn(CC(F)F)c4n3)c12 ; CNc1nc(N[C@@H]2CCN(C3(C)COC3)CC2(F)F)nn2ccc(-c3ccc4nnn(CC(F)F)c4n3)c12] 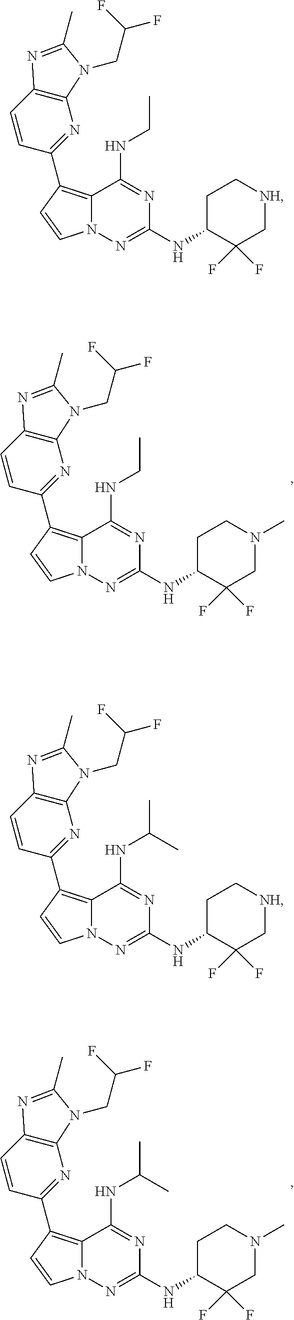 [SMILES: CCNc1nc(N[C@@H]2CCNCC2(F)F)nn2ccc(-c3ccc4nc(C)n(CC(F)F)c4n3)c12 ; CCNc1nc(N[C@@H]2CCN(C)CC2(F)F)nn2ccc(-c3ccc4nc(C)n(CC(F)F)c4n3)c12 ; Cc1nc2ccc(-c3ccn4nc(N[C@@H]5CCNCC5(F)F)nc(NC(C)C)c34)nc2n1CC(F)F ; Cc1nc2ccc(-c3ccn4nc(N[C@@H]5CCN(C)CC5(F)F)nc(NC(C)C)c34)nc2n1CC(F)F] 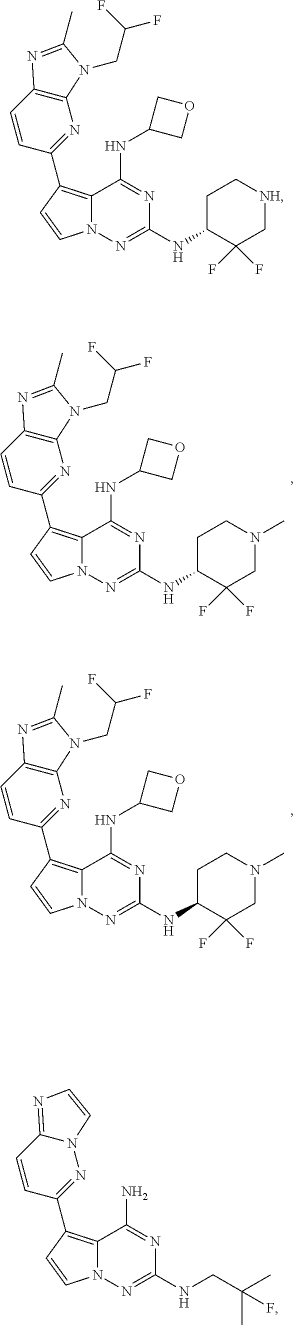 [SMILES: Cc1nc2ccc(-c3ccn4nc(N[C@@H]5CCNCC5(F)F)nc(NC5COC5)c34)nc2n1CC(F)F ; Cc1nc2ccc(-c3ccn4nc(N[C@@H]5CCN(C)CC5(F)F)nc(NC5COC5)c34)nc2n1CC(F)F ; Cc1nc2ccc(-c3ccn4nc(N[C@H]5CCN(C)CC5(F)F)nc(NC5COC5)c34)nc2n1CC(F)F ; CC(C)(F)CNc1nc(N)c2c(-c3ccc4nccn4n3)ccn2n1] 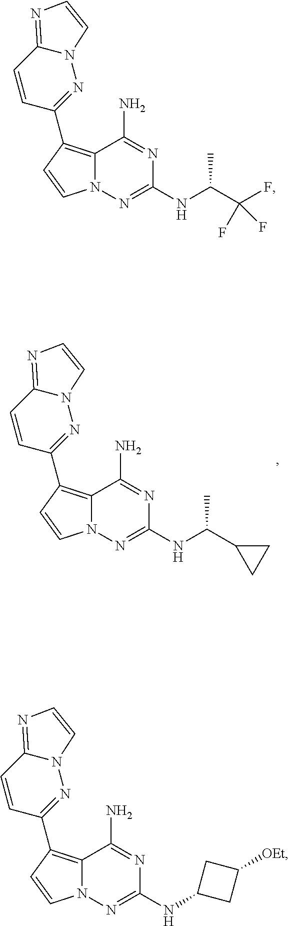 [SMILES: C[C@@H](Nc1nc(N)c2c(-c3ccc4nccn4n3)ccn2n1)C(F)(F)F ; C[C@@H](Nc1nc(N)c2c(-c3ccc4nccn4n3)ccn2n1)C1CC1 ; CCO[C@H]1C[C@@H](Nc2nc(N)c3c(-c4ccc5nccn5n4)ccn3n2)C1] 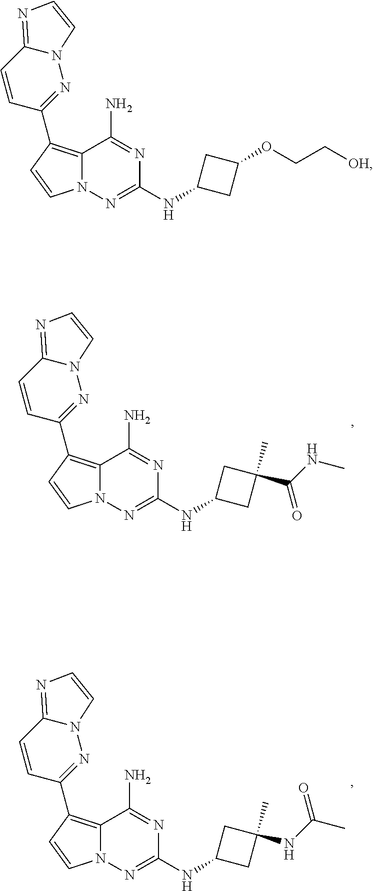 [SMILES: Nc1nc(N[C@H]2C[C@@H](OCCO)C2)nn2ccc(-c3ccc4nccn4n3)c12 ; CNC(=O)[C@]1(C)C[C@@H](Nc2nc(N)c3c(-c4ccc5nccn5n4)ccn3n2)C1 ; CC(=O)N[C@]1(C)C[C@@H](Nc2nc(N)c3c(-c4ccc5nccn5n4)ccn3n2)C1] 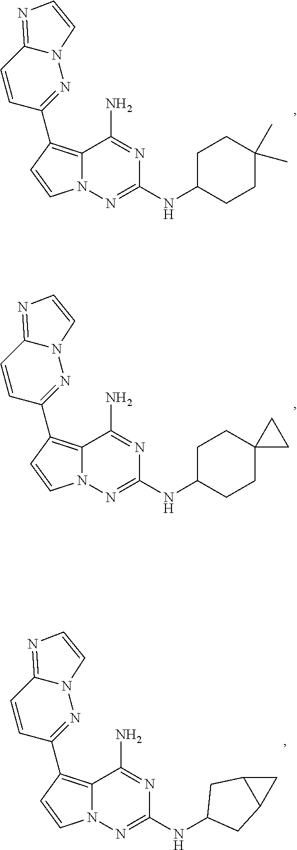 [SMILES: CC1(C)CCC(Nc2nc(N)c3c(-c4ccc5nccn5n4)ccn3n2)CC1 ; Nc1nc(NC2CCC3(CC2)CC3)nn2ccc(-c3ccc4nccn4n3)c12 ; Nc1nc(NC2CC3CC3C2)nn2ccc(-c3ccc4nccn4n3)c12] 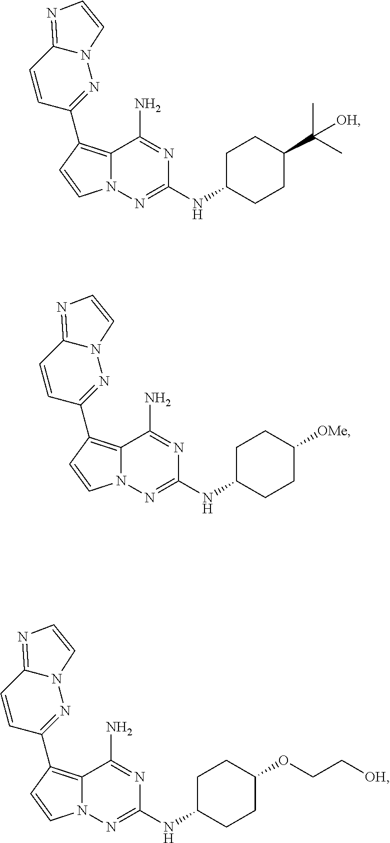 [SMILES: CC(C)(O)[C@H]1CC[C@H](Nc2nc(N)c3c(-c4ccc5nccn5n4)ccn3n2)CC1 ; CO[C@H]1CC[C@@H](Nc2nc(N)c3c(-c4ccc5nccn5n4)ccn3n2)CC1 ; Nc1nc(N[C@H]2CC[C@@H](OCCO)CC2)nn2ccc(-c3ccc4nccn4n3)c12] 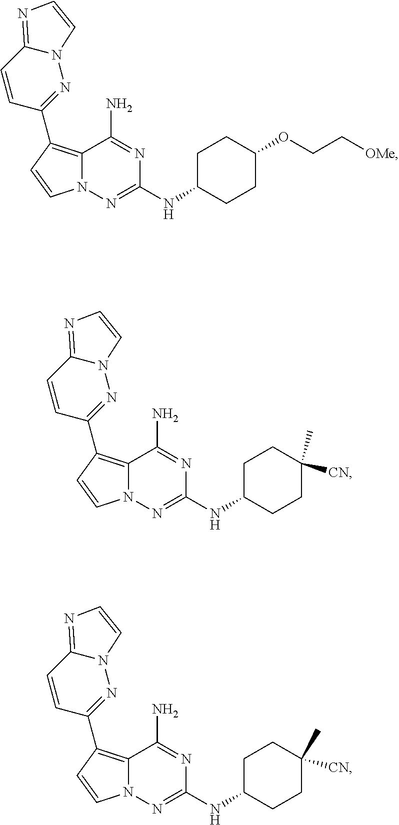 [SMILES: COCCO[C@H]1CC[C@@H](Nc2nc(N)c3c(-c4ccc5nccn5n4)ccn3n2)CC1 ; C[C@]1(C#N)CC[C@H](Nc2nc(N)c3c(-c4ccc5nccn5n4)ccn3n2)CC1 ; C[C@]1(C#N)CC[C@@H](Nc2nc(N)c3c(-c4ccc5nccn5n4)ccn3n2)CC1] 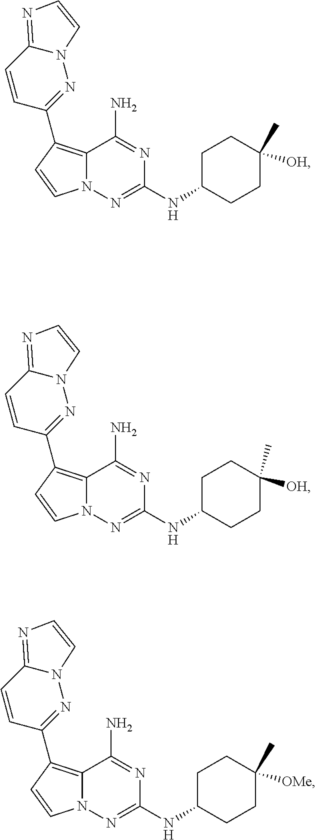 [SMILES: C[C@]1(O)CC[C@@H](Nc2nc(N)c3c(-c4ccc5nccn5n4)ccn3n2)CC1 ; C[C@]1(O)CC[C@H](Nc2nc(N)c3c(-c4ccc5nccn5n4)ccn3n2)CC1 ; CO[C@]1(C)CC[C@H](Nc2nc(N)c3c(-c4ccc5nccn5n4)ccn3n2)CC1] 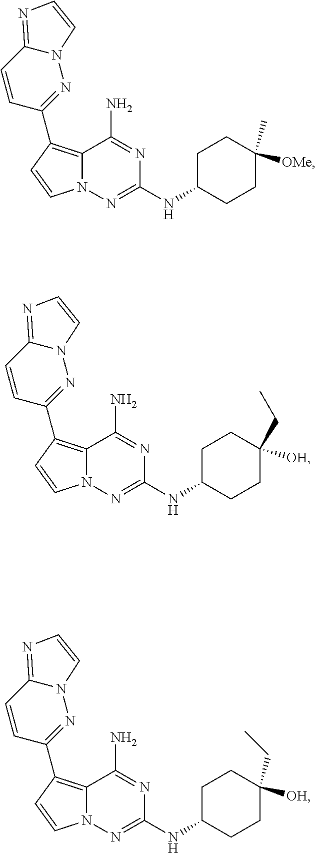 [SMILES: CO[C@]1(C)CC[C@@H](Nc2nc(N)c3c(-c4ccc5nccn5n4)ccn3n2)CC1 ; CC[C@]1(O)CC[C@@H](Nc2nc(N)c3c(-c4ccc5nccn5n4)ccn3n2)CC1 ; CC[C@]1(O)CC[C@H](Nc2nc(N)c3c(-c4ccc5nccn5n4)ccn3n2)CC1] 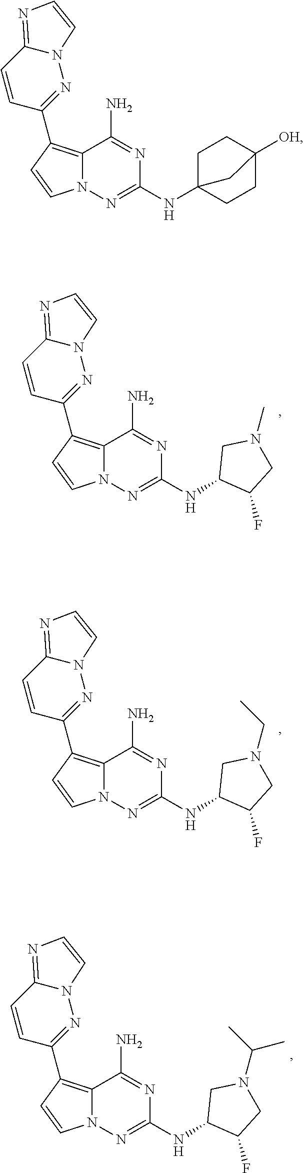 [SMILES: Nc1nc(NC23CCC(O)(CC2)C3)nn2ccc(-c3ccc4nccn4n3)c12 ; CN1C[C@H](F)[C@H](Nc2nc(N)c3c(-c4ccc5nccn5n4)ccn3n2)C1 ; CCN1C[C@H](F)[C@H](Nc2nc(N)c3c(-c4ccc5nccn5n4)ccn3n2)C1 ; CC(C)N1C[C@H](F)[C@H](Nc2nc(N)c3c(-c4ccc5nccn5n4)ccn3n2)C1] 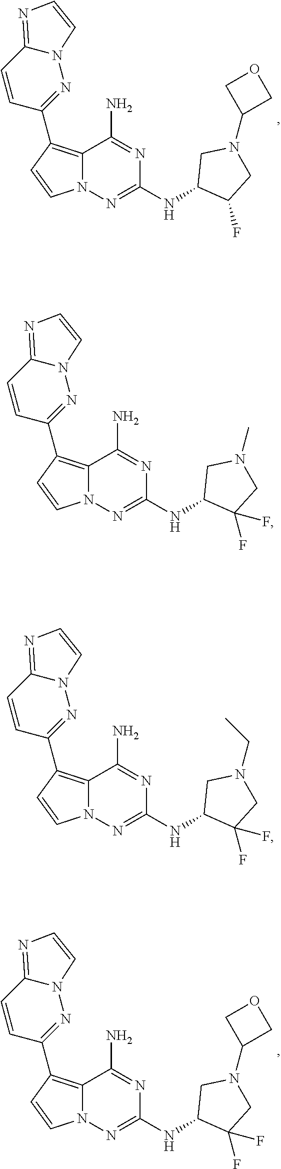 [SMILES: Nc1nc(N[C@@H]2CN(C3COC3)C[C@@H]2F)nn2ccc(-c3ccc4nccn4n3)c12 ; CN1C[C@@H](Nc2nc(N)c3c(-c4ccc5nccn5n4)ccn3n2)C(F)(F)C1 ; CCN1C[C@@H](Nc2nc(N)c3c(-c4ccc5nccn5n4)ccn3n2)C(F)(F)C1 ; Nc1nc(N[C@@H]2CN(C3COC3)CC2(F)F)nn2ccc(-c3ccc4nccn4n3)c12] 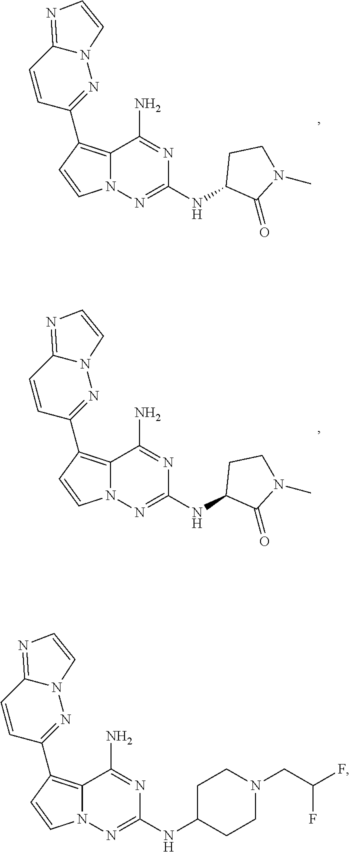 [SMILES: CN1CC[C@@H](Nc2nc(N)c3c(-c4ccc5nccn5n4)ccn3n2)C1=O ; CN1CC[C@H](Nc2nc(N)c3c(-c4ccc5nccn5n4)ccn3n2)C1=O ; Nc1nc(NC2CCN(CC(F)F)CC2)nn2ccc(-c3ccc4nccn4n3)c12] 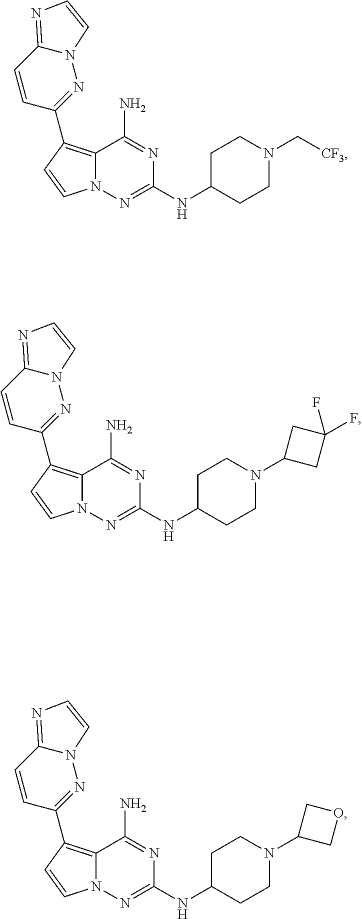 [SMILES: Nc1nc(NC2CCN(CC(F)(F)F)CC2)nn2ccc(-c3ccc4nccn4n3)c12 ; Nc1nc(NC2CCN(C3CC(F)(F)C3)CC2)nn2ccc(-c3ccc4nccn4n3)c12 ; Nc1nc(NC2CCN(C3COC3)CC2)nn2ccc(-c3ccc4nccn4n3)c12] 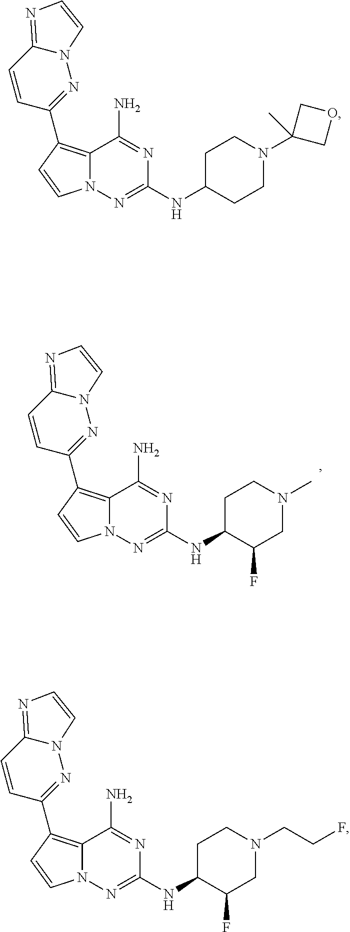 [SMILES: CC1(N2CCC(Nc3nc(N)c4c(-c5ccc6nccn6n5)ccn4n3)CC2)COC1 ; CN1CC[C@H](Nc2nc(N)c3c(-c4ccc5nccn5n4)ccn3n2)[C@H](F)C1 ; Nc1nc(N[C@H]2CCN(CCF)C[C@H]2F)nn2ccc(-c3ccc4nccn4n3)c12] 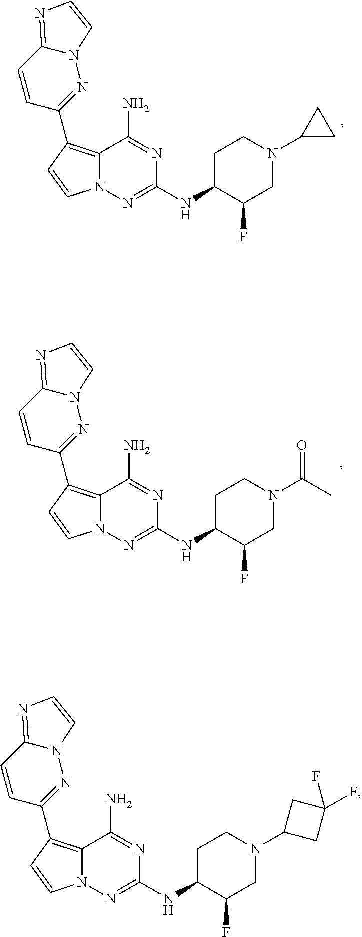 [SMILES: Nc1nc(N[C@H]2CCN(C3CC3)C[C@H]2F)nn2ccc(-c3ccc4nccn4n3)c12 ; CC(=O)N1CC[C@H](Nc2nc(N)c3c(-c4ccc5nccn5n4)ccn3n2)[C@H](F)C1 ; Nc1nc(N[C@H]2CCN(C3CC(F)(F)C3)C[C@H]2F)nn2ccc(-c3ccc4nccn4n3)c12] 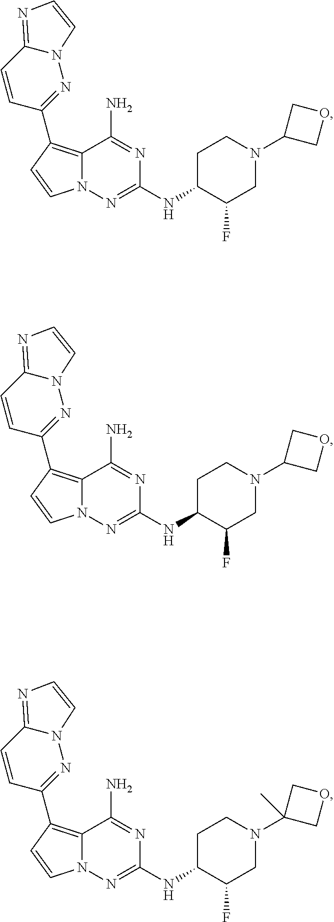 [SMILES: Nc1nc(N[C@@H]2CCN(C3COC3)C[C@@H]2F)nn2ccc(-c3ccc4nccn4n3)c12 ; Nc1nc(N[C@H]2CCN(C3COC3)C[C@H]2F)nn2ccc(-c3ccc4nccn4n3)c12 ; CC1(N2CC[C@@H](Nc3nc(N)c4c(-c5ccc6nccn6n5)ccn4n3)[C@@H](F)C2)COC1] 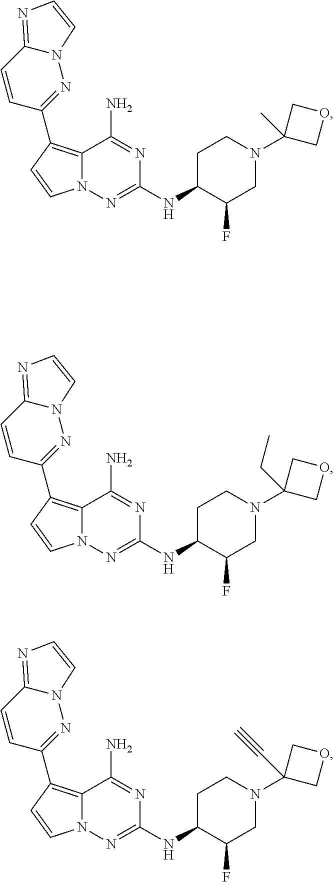 [SMILES: CC1(N2CC[C@H](Nc3nc(N)c4c(-c5ccc6nccn6n5)ccn4n3)[C@H](F)C2)COC1 ; CCC1(N2CC[C@H](Nc3nc(N)c4c(-c5ccc6nccn6n5)ccn4n3)[C@H](F)C2)COC1 ; C#CC1(N2CC[C@H](Nc3nc(N)c4c(-c5ccc6nccn6n5)ccn4n3)[C@H](F)C2)COC1] 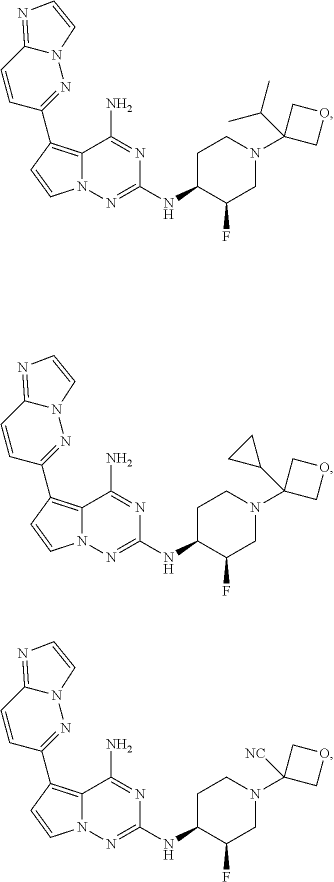 [SMILES: CC(C)C1(N2CC[C@H](Nc3nc(N)c4c(-c5ccc6nccn6n5)ccn4n3)[C@H](F)C2)COC1 ; Nc1nc(N[C@H]2CCN(C3(C4CC4)COC3)C[C@H]2F)nn2ccc(-c3ccc4nccn4n3)c12 ; N#CC1(N2CC[C@H](Nc3nc(N)c4c(-c5ccc6nccn6n5)ccn4n3)[C@H](F)C2)COC1] 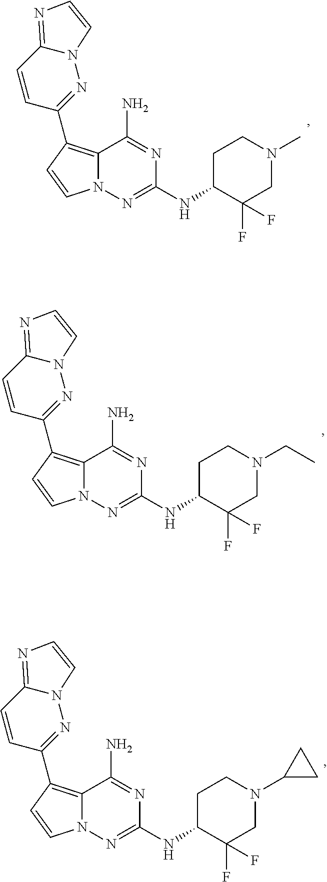 [SMILES: CN1CC[C@@H](Nc2nc(N)c3c(-c4ccc5nccn5n4)ccn3n2)C(F)(F)C1 ; CCN1CC[C@@H](Nc2nc(N)c3c(-c4ccc5nccn5n4)ccn3n2)C(F)(F)C1 ; Nc1nc(N[C@@H]2CCN(C3CC3)CC2(F)F)nn2ccc(-c3ccc4nccn4n3)c12] 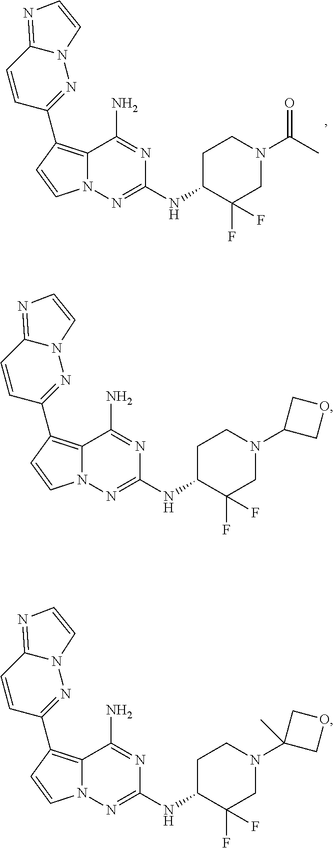 [SMILES: CC(=O)N1CC[C@@H](Nc2nc(N)c3c(-c4ccc5nccn5n4)ccn3n2)C(F)(F)C1 ; Nc1nc(N[C@@H]2CCN(C3COC3)CC2(F)F)nn2ccc(-c3ccc4nccn4n3)c12 ; CC1(N2CC[C@@H](Nc3nc(N)c4c(-c5ccc6nccn6n5)ccn4n3)C(F)(F)C2)COC1] 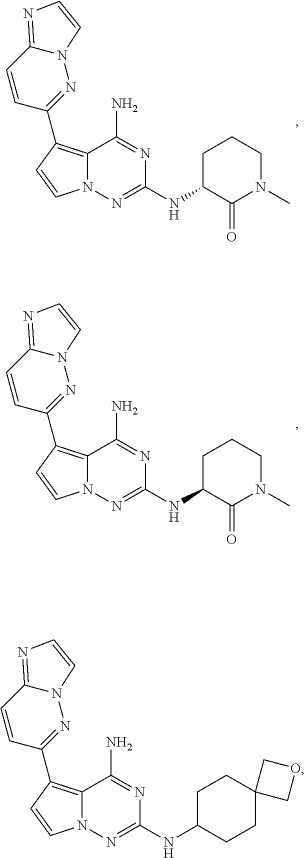 [SMILES: CN1CCC[C@@H](Nc2nc(N)c3c(-c4ccc5nccn5n4)ccn3n2)C1=O ; CN1CCC[C@H](Nc2nc(N)c3c(-c4ccc5nccn5n4)ccn3n2)C1=O ; Nc1nc(NC2CCC3(CC2)COC3)nn2ccc(-c3ccc4nccn4n3)c12] 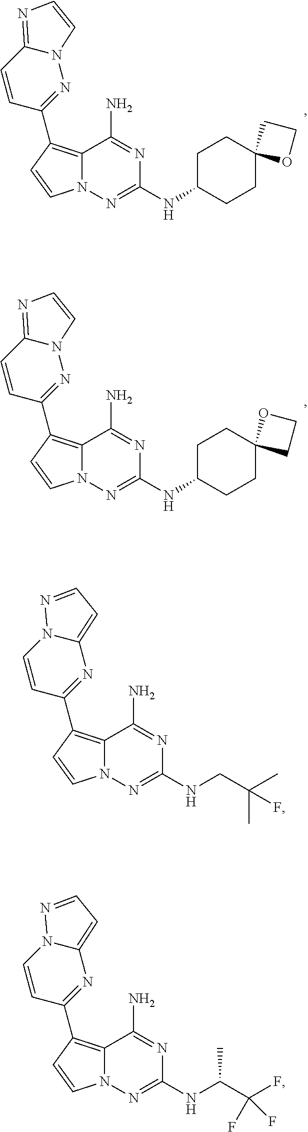 [SMILES: Nc1nc(N[C@H]2CC[C@@]3(CCO3)CC2)nn2ccc(-c3ccc4nccn4n3)c12 ; Nc1nc(N[C@H]2CC[C@]3(CCO3)CC2)nn2ccc(-c3ccc4nccn4n3)c12 ; CC(C)(F)CNc1nc(N)c2c(-c3ccn4nccc4n3)ccn2n1 ; C[C@@H](Nc1nc(N)c2c(-c3ccn4nccc4n3)ccn2n1)C(F)(F)F] 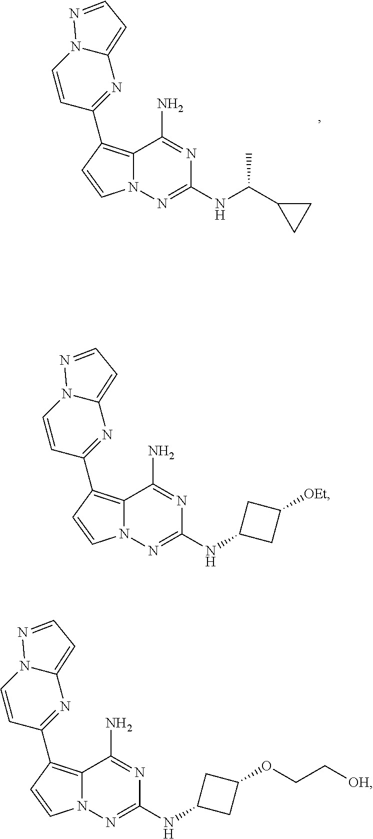 [SMILES: C[C@@H](Nc1nc(N)c2c(-c3ccn4nccc4n3)ccn2n1)C1CC1 ; CCO[C@H]1C[C@@H](Nc2nc(N)c3c(-c4ccn5nccc5n4)ccn3n2)C1 ; Nc1nc(N[C@H]2C[C@@H](OCCO)C2)nn2ccc(-c3ccn4nccc4n3)c12] 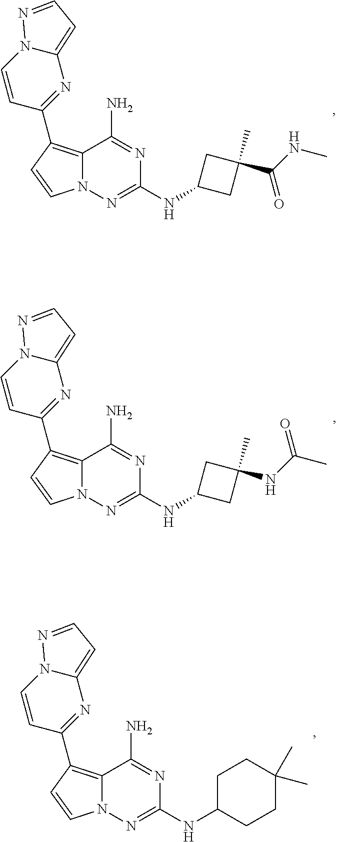 [SMILES: CNC(=O)[C@]1(C)C[C@@H](Nc2nc(N)c3c(-c4ccn5nccc5n4)ccn3n2)C1 ; CC(=O)N[C@]1(C)C[C@@H](Nc2nc(N)c3c(-c4ccn5nccc5n4)ccn3n2)C1 ; CC1(C)CCC(Nc2nc(N)c3c(-c4ccn5nccc5n4)ccn3n2)CC1] 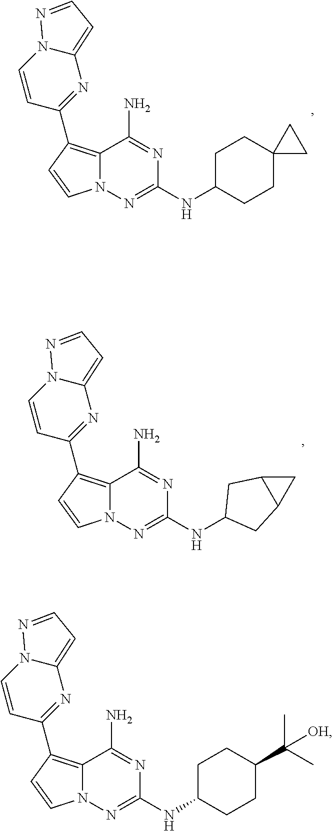 [SMILES: Nc1nc(NC2CCC3(CC2)CC3)nn2ccc(-c3ccn4nccc4n3)c12 ; Nc1nc(NC2CC3CC3C2)nn2ccc(-c3ccn4nccc4n3)c12 ; CC(C)(O)[C@H]1CC[C@H](Nc2nc(N)c3c(-c4ccn5nccc5n4)ccn3n2)CC1] 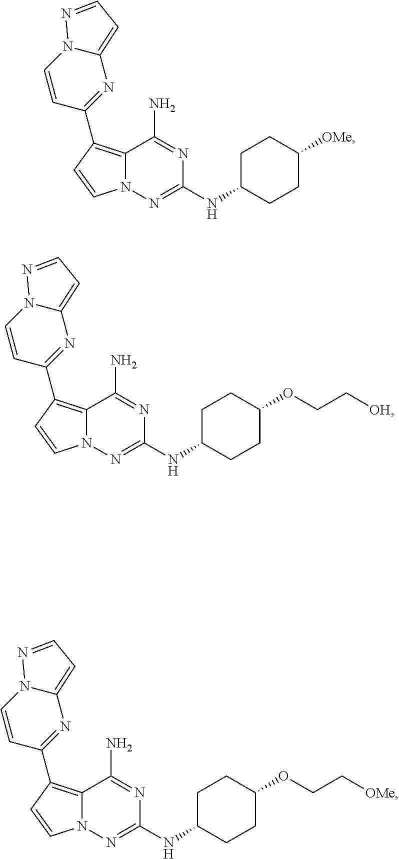 [SMILES: CO[C@H]1CC[C@@H](Nc2nc(N)c3c(-c4ccn5nccc5n4)ccn3n2)CC1 ; Nc1nc(N[C@H]2CC[C@@H](OCCO)CC2)nn2ccc(-c3ccn4nccc4n3)c12 ; COCCO[C@H]1CC[C@@H](Nc2nc(N)c3c(-c4ccn5nccc5n4)ccn3n2)CC1] 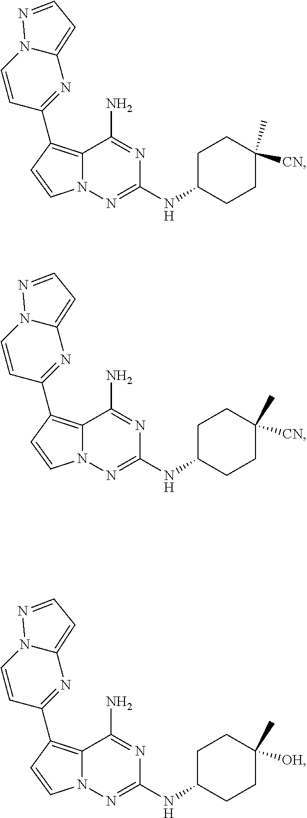 [SMILES: C[C@]1(C#N)CC[C@H](Nc2nc(N)c3c(-c4ccn5nccc5n4)ccn3n2)CC1 ; C[C@]1(C#N)CC[C@@H](Nc2nc(N)c3c(-c4ccn5nccc5n4)ccn3n2)CC1 ; C[C@]1(O)CC[C@@H](Nc2nc(N)c3c(-c4ccn5nccc5n4)ccn3n2)CC1] 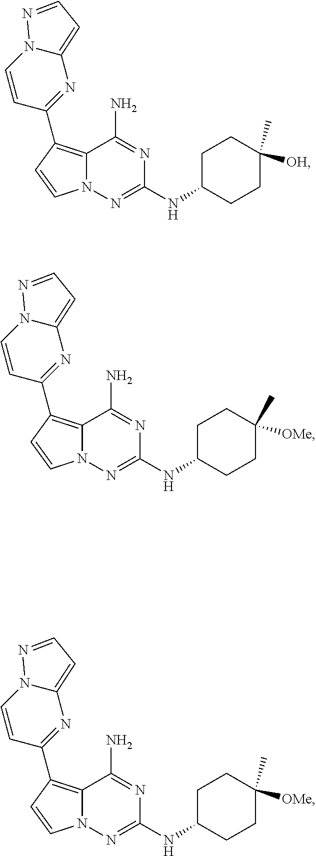 [SMILES: C[C@]1(O)CC[C@H](Nc2nc(N)c3c(-c4ccn5nccc5n4)ccn3n2)CC1 ; CO[C@]1(C)CC[C@H](Nc2nc(N)c3c(-c4ccn5nccc5n4)ccn3n2)CC1 ; CO[C@]1(C)CC[C@@H](Nc2nc(N)c3c(-c4ccn5nccc5n4)ccn3n2)CC1] 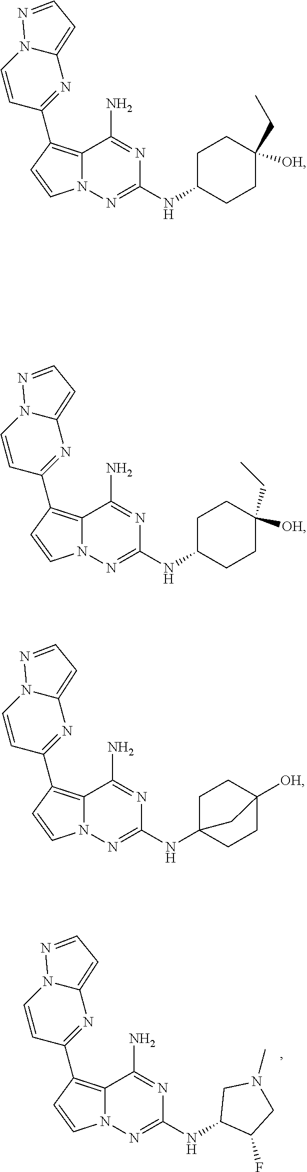 [SMILES: CC[C@]1(O)CC[C@@H](Nc2nc(N)c3c(-c4ccn5nccc5n4)ccn3n2)CC1 ; CC[C@]1(O)CC[C@H](Nc2nc(N)c3c(-c4ccn5nccc5n4)ccn3n2)CC1 ; Nc1nc(NC23CCC(O)(CC2)C3)nn2ccc(-c3ccn4nccc4n3)c12 ; CN1C[C@H](F)[C@H](Nc2nc(N)c3c(-c4ccn5nccc5n4)ccn3n2)C1] 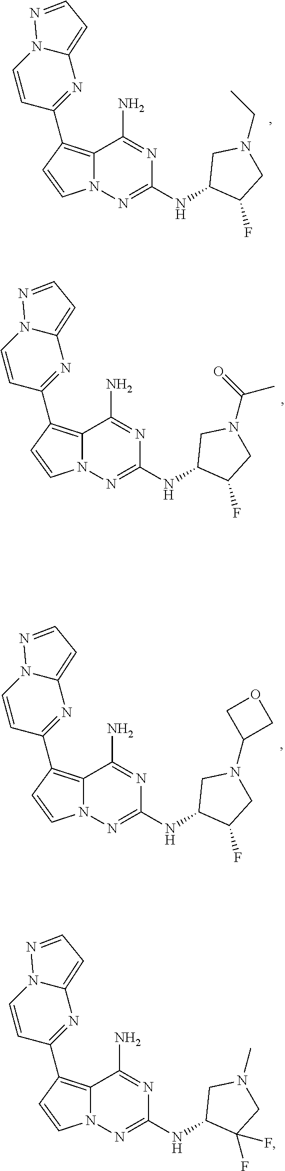 [SMILES: CCN1C[C@H](F)[C@H](Nc2nc(N)c3c(-c4ccn5nccc5n4)ccn3n2)C1 ; CC(=O)N1C[C@H](F)[C@H](Nc2nc(N)c3c(-c4ccn5nccc5n4)ccn3n2)C1 ; Nc1nc(N[C@@H]2CN(C3COC3)C[C@@H]2F)nn2ccc(-c3ccn4nccc4n3)c12 ; CN1C[C@@H](Nc2nc(N)c3c(-c4ccn5nccc5n4)ccn3n2)C(F)(F)C1] 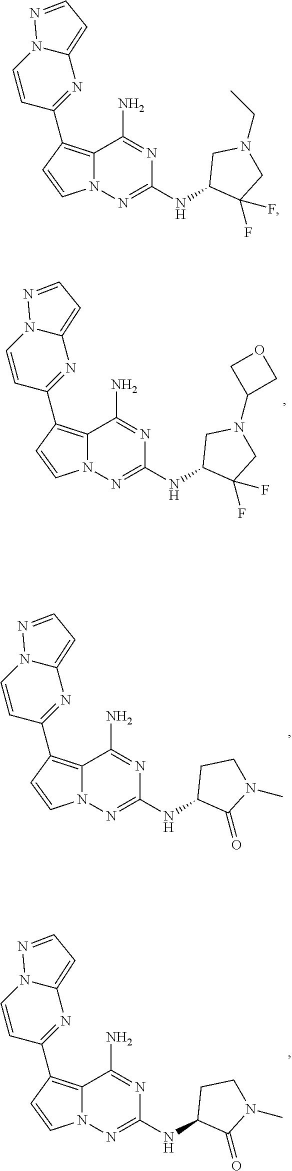 [SMILES: CCN1C[C@@H](Nc2nc(N)c3c(-c4ccn5nccc5n4)ccn3n2)C(F)(F)C1 ; Nc1nc(N[C@@H]2CN(C3COC3)CC2(F)F)nn2ccc(-c3ccn4nccc4n3)c12 ; CN1CC[C@@H](Nc2nc(N)c3c(-c4ccn5nccc5n4)ccn3n2)C1=O ; CN1CC[C@H](Nc2nc(N)c3c(-c4ccn5nccc5n4)ccn3n2)C1=O] 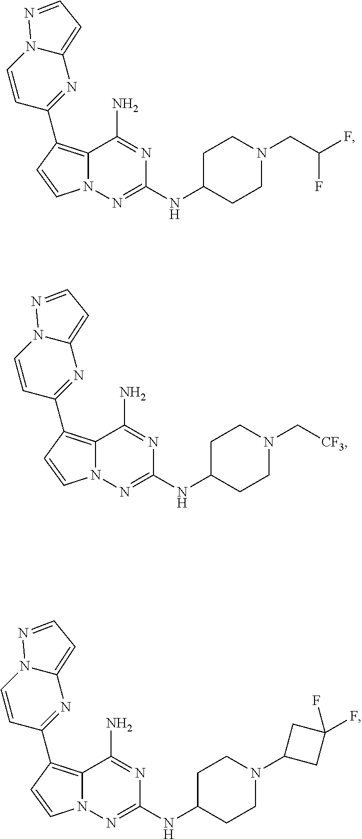 [SMILES: Nc1nc(NC2CCN(CC(F)F)CC2)nn2ccc(-c3ccn4nccc4n3)c12 ; Nc1nc(NC2CCN(CC(F)(F)F)CC2)nn2ccc(-c3ccn4nccc4n3)c12 ; Nc1nc(NC2CCN(C3CC(F)(F)C3)CC2)nn2ccc(-c3ccn4nccc4n3)c12] 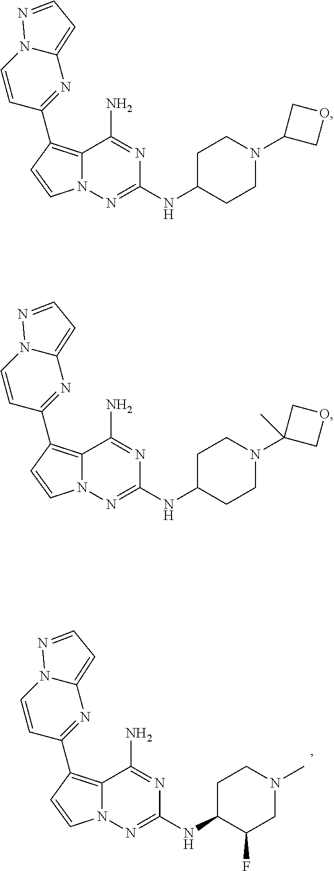 [SMILES: Nc1nc(NC2CCN(C3COC3)CC2)nn2ccc(-c3ccn4nccc4n3)c12 ; CC1(N2CCC(Nc3nc(N)c4c(-c5ccn6nccc6n5)ccn4n3)CC2)COC1 ; CN1CC[C@H](Nc2nc(N)c3c(-c4ccn5nccc5n4)ccn3n2)[C@H](F)C1] 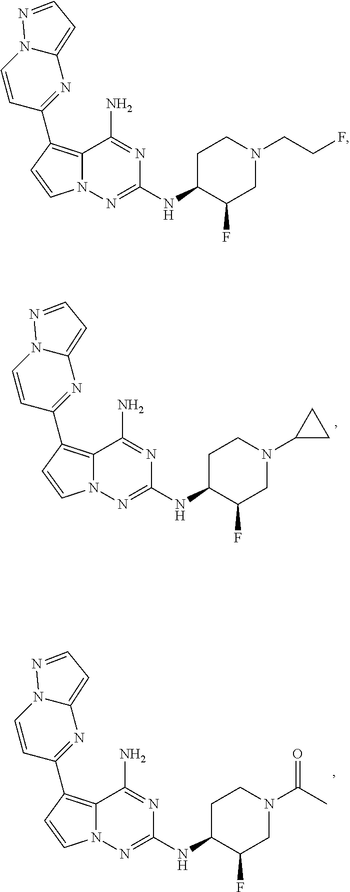 [SMILES: Nc1nc(N[C@H]2CCN(CCF)C[C@H]2F)nn2ccc(-c3ccn4nccc4n3)c12 ; Nc1nc(N[C@H]2CCN(C3CC3)C[C@H]2F)nn2ccc(-c3ccn4nccc4n3)c12 ; CC(=O)N1CC[C@H](Nc2nc(N)c3c(-c4ccn5nccc5n4)ccn3n2)[C@H](F)C1] 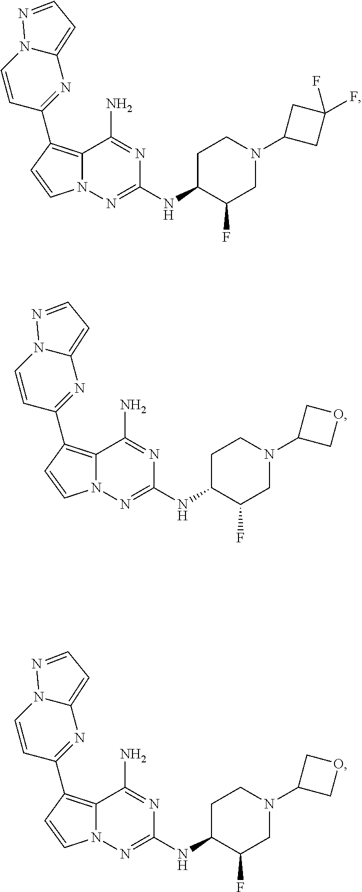 [SMILES: Nc1nc(N[C@H]2CCN(C3CC(F)(F)C3)C[C@H]2F)nn2ccc(-c3ccn4nccc4n3)c12 ; Nc1nc(N[C@@H]2CCN(C3COC3)C[C@@H]2F)nn2ccc(-c3ccn4nccc4n3)c12 ; Nc1nc(N[C@H]2CCN(C3COC3)C[C@H]2F)nn2ccc(-c3ccn4nccc4n3)c12] 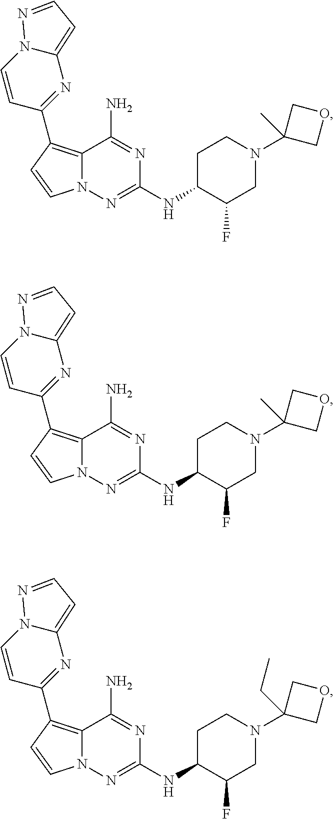 [SMILES: CC1(N2CC[C@@H](Nc3nc(N)c4c(-c5ccn6nccc6n5)ccn4n3)[C@@H](F)C2)COC1 ; CC1(N2CC[C@H](Nc3nc(N)c4c(-c5ccn6nccc6n5)ccn4n3)[C@H](F)C2)COC1 ; CCC1(N2CC[C@H](Nc3nc(N)c4c(-c5ccn6nccc6n5)ccn4n3)[C@H](F)C2)COC1] 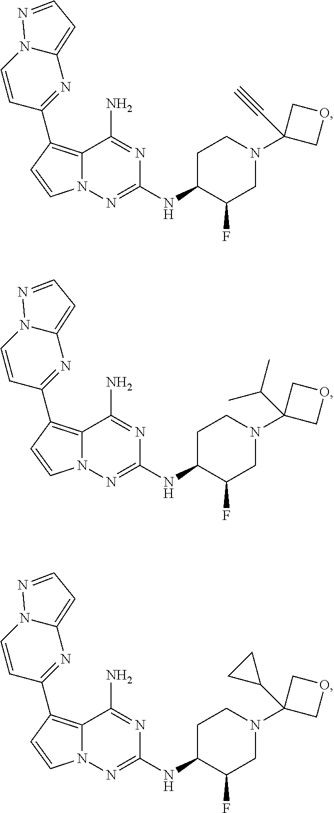 [SMILES: C#CC1(N2CC[C@H](Nc3nc(N)c4c(-c5ccn6nccc6n5)ccn4n3)[C@H](F)C2)COC1 ; CC(C)C1(N2CC[C@H](Nc3nc(N)c4c(-c5ccn6nccc6n5)ccn4n3)[C@H](F)C2)COC1 ; Nc1nc(N[C@H]2CCN(C3(C4CC4)COC3)C[C@H]2F)nn2ccc(-c3ccn4nccc4n3)c12] 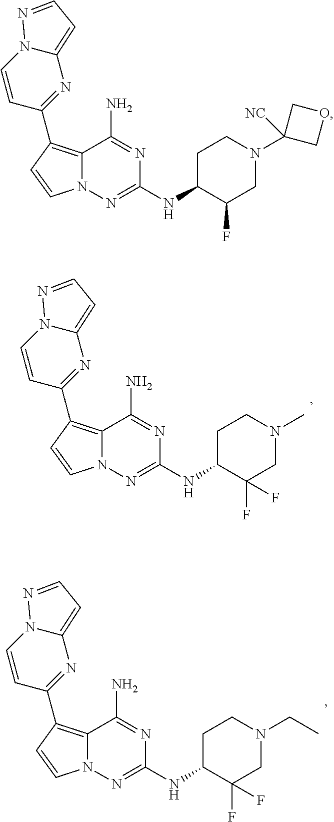 [SMILES: N#CC1(N2CC[C@H](Nc3nc(N)c4c(-c5ccn6nccc6n5)ccn4n3)[C@H](F)C2)COC1 ; CN1CC[C@@H](Nc2nc(N)c3c(-c4ccn5nccc5n4)ccn3n2)C(F)(F)C1 ; CCN1CC[C@@H](Nc2nc(N)c3c(-c4ccn5nccc5n4)ccn3n2)C(F)(F)C1] 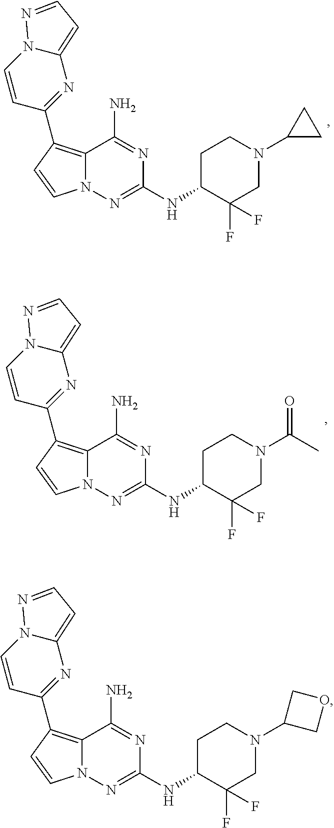 [SMILES: Nc1nc(N[C@@H]2CCN(C3CC3)CC2(F)F)nn2ccc(-c3ccn4nccc4n3)c12 ; CC(=O)N1CC[C@@H](Nc2nc(N)c3c(-c4ccn5nccc5n4)ccn3n2)C(F)(F)C1 ; Nc1nc(N[C@@H]2CCN(C3COC3)CC2(F)F)nn2ccc(-c3ccn4nccc4n3)c12] 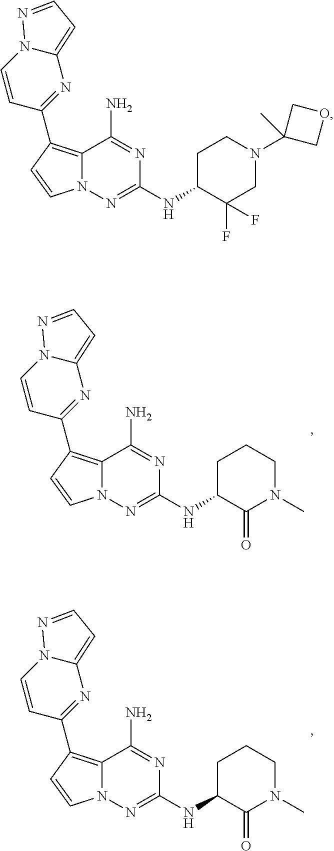 [SMILES: CC1(N2CC[C@@H](Nc3nc(N)c4c(-c5ccn6nccc6n5)ccn4n3)C(F)(F)C2)COC1 ; CN1CCC[C@@H](Nc2nc(N)c3c(-c4ccn5nccc5n4)ccn3n2)C1=O ; CN1CCC[C@H](Nc2nc(N)c3c(-c4ccn5nccc5n4)ccn3n2)C1=O] 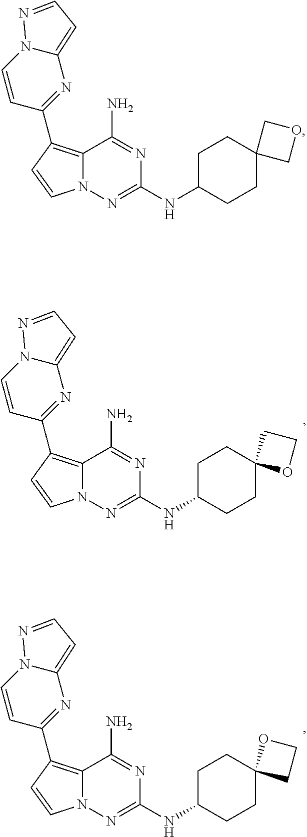 [SMILES: Nc1nc(NC2CCC3(CC2)COC3)nn2ccc(-c3ccn4nccc4n3)c12 ; Nc1nc(N[C@H]2CC[C@@]3(CCO3)CC2)nn2ccc(-c3ccn4nccc4n3)c12 ; Nc1nc(N[C@H]2CC[C@]3(CCO3)CC2)nn2ccc(-c3ccn4nccc4n3)c12] 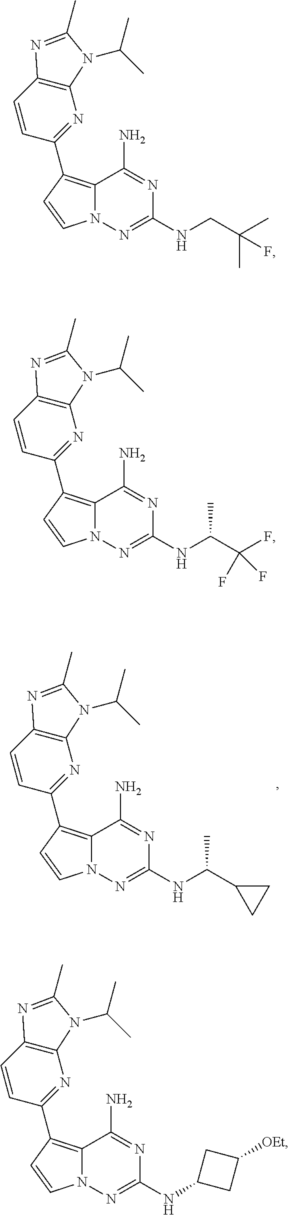 [SMILES: Cc1nc2ccc(-c3ccn4nc(NCC(C)(C)F)nc(N)c34)nc2n1C(C)C ; Cc1nc2ccc(-c3ccn4nc(N[C@H](C)C(F)(F)F)nc(N)c34)nc2n1C(C)C ; Cc1nc2ccc(-c3ccn4nc(N[C@H](C)C5CC5)nc(N)c34)nc2n1C(C)C ; CCO[C@H]1C[C@@H](Nc2nc(N)c3c(-c4ccc5nc(C)n(C(C)C)c5n4)ccn3n2)C1] 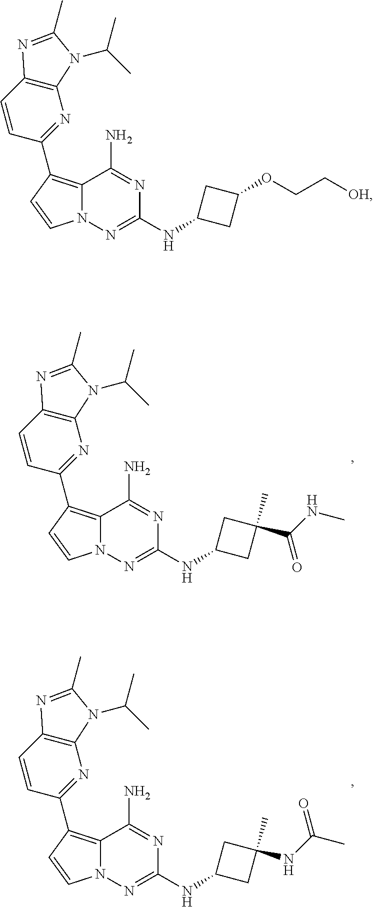 [SMILES: Cc1nc2ccc(-c3ccn4nc(N[C@H]5C[C@@H](OCCO)C5)nc(N)c34)nc2n1C(C)C ; CNC(=O)[C@]1(C)C[C@@H](Nc2nc(N)c3c(-c4ccc5nc(C)n(C(C)C)c5n4)ccn3n2)C1 ; CC(=O)N[C@]1(C)C[C@@H](Nc2nc(N)c3c(-c4ccc5nc(C)n(C(C)C)c5n4)ccn3n2)C1] 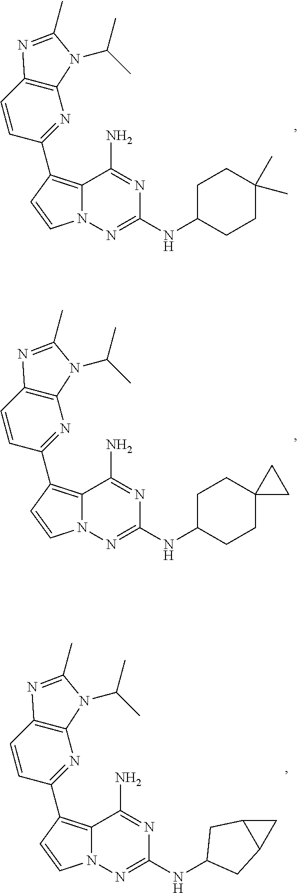 [SMILES: Cc1nc2ccc(-c3ccn4nc(NC5CCC(C)(C)CC5)nc(N)c34)nc2n1C(C)C ; Cc1nc2ccc(-c3ccn4nc(NC5CCC6(CC5)CC6)nc(N)c34)nc2n1C(C)C ; Cc1nc2ccc(-c3ccn4nc(NC5CC6CC6C5)nc(N)c34)nc2n1C(C)C] 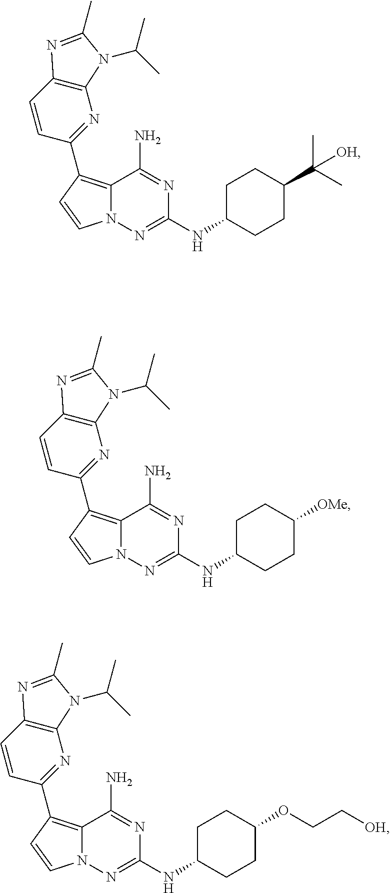 [SMILES: Cc1nc2ccc(-c3ccn4nc(N[C@H]5CC[C@H](C(C)(C)O)CC5)nc(N)c34)nc2n1C(C)C ; CO[C@H]1CC[C@@H](Nc2nc(N)c3c(-c4ccc5nc(C)n(C(C)C)c5n4)ccn3n2)CC1 ; Cc1nc2ccc(-c3ccn4nc(N[C@H]5CC[C@@H](OCCO)CC5)nc(N)c34)nc2n1C(C)C] 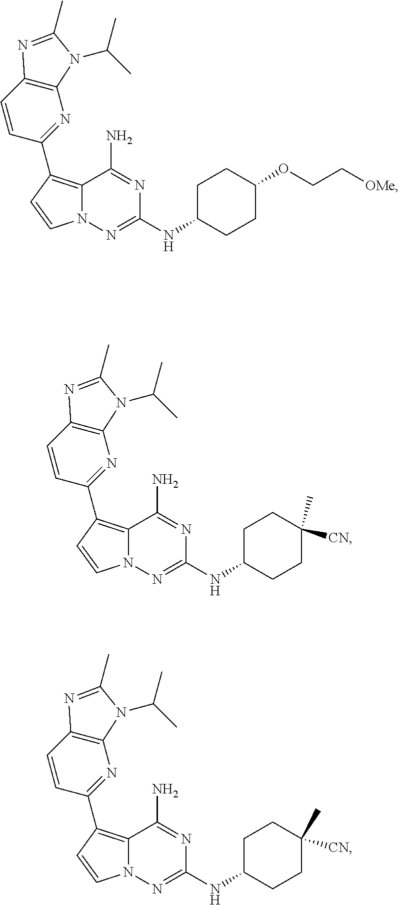 [SMILES: COCCO[C@H]1CC[C@@H](Nc2nc(N)c3c(-c4ccc5nc(C)n(C(C)C)c5n4)ccn3n2)CC1 ; Cc1nc2ccc(-c3ccn4nc(N[C@H]5CC[C@](C)(C#N)CC5)nc(N)c34)nc2n1C(C)C ; Cc1nc2ccc(-c3ccn4nc(N[C@H]5CC[C@@](C)(C#N)CC5)nc(N)c34)nc2n1C(C)C] 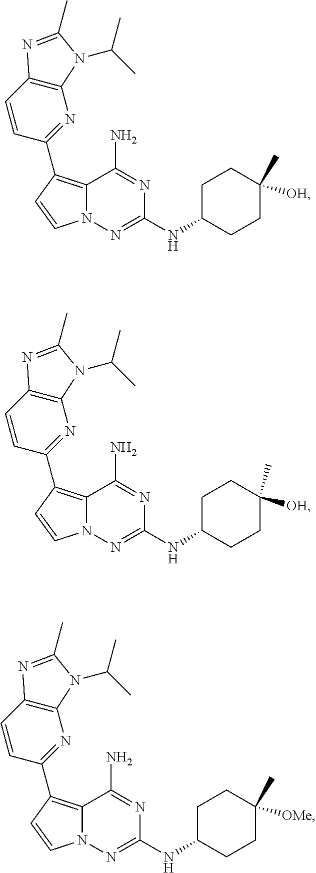 [SMILES: Cc1nc2ccc(-c3ccn4nc(N[C@H]5CC[C@@](C)(O)CC5)nc(N)c34)nc2n1C(C)C ; Cc1nc2ccc(-c3ccn4nc(N[C@H]5CC[C@](C)(O)CC5)nc(N)c34)nc2n1C(C)C ; CO[C@]1(C)CC[C@H](Nc2nc(N)c3c(-c4ccc5nc(C)n(C(C)C)c5n4)ccn3n2)CC1] 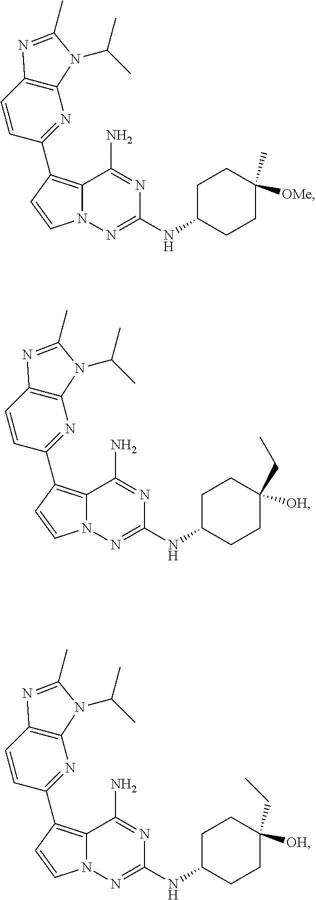 [SMILES: CO[C@]1(C)CC[C@@H](Nc2nc(N)c3c(-c4ccc5nc(C)n(C(C)C)c5n4)ccn3n2)CC1 ; CC[C@]1(O)CC[C@@H](Nc2nc(N)c3c(-c4ccc5nc(C)n(C(C)C)c5n4)ccn3n2)CC1 ; CC[C@]1(O)CC[C@H](Nc2nc(N)c3c(-c4ccc5nc(C)n(C(C)C)c5n4)ccn3n2)CC1] 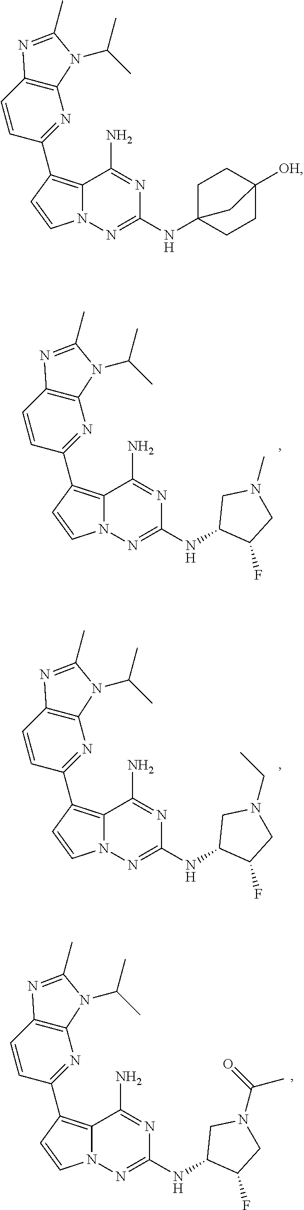 [SMILES: Cc1nc2ccc(-c3ccn4nc(NC56CCC(O)(CC5)C6)nc(N)c34)nc2n1C(C)C ; Cc1nc2ccc(-c3ccn4nc(N[C@@H]5CN(C)C[C@@H]5F)nc(N)c34)nc2n1C(C)C ; CCN1C[C@H](F)[C@H](Nc2nc(N)c3c(-c4ccc5nc(C)n(C(C)C)c5n4)ccn3n2)C1 ; CC(=O)N1C[C@H](F)[C@H](Nc2nc(N)c3c(-c4ccc5nc(C)n(C(C)C)c5n4)ccn3n2)C1] 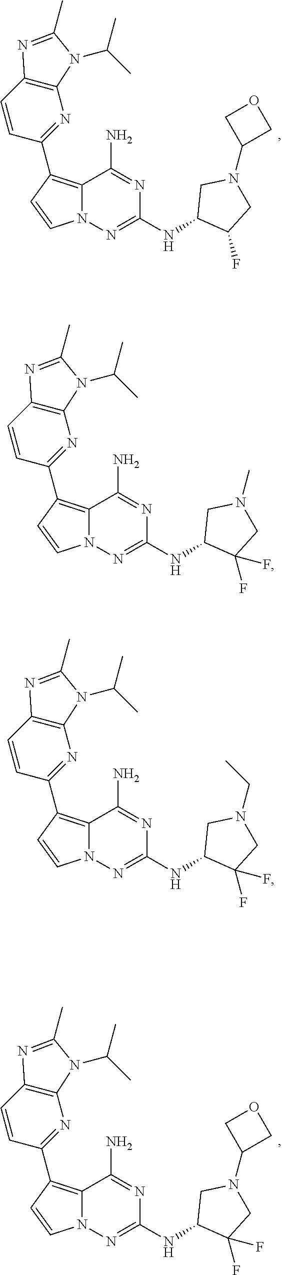 [SMILES: Cc1nc2ccc(-c3ccn4nc(N[C@@H]5CN(C6COC6)C[C@@H]5F)nc(N)c34)nc2n1C(C)C ; Cc1nc2ccc(-c3ccn4nc(N[C@@H]5CN(C)CC5(F)F)nc(N)c34)nc2n1C(C)C ; CCN1C[C@@H](Nc2nc(N)c3c(-c4ccc5nc(C)n(C(C)C)c5n4)ccn3n2)C(F)(F)C1 ; Cc1nc2ccc(-c3ccn4nc(N[C@@H]5CN(C6COC6)CC5(F)F)nc(N)c34)nc2n1C(C)C] 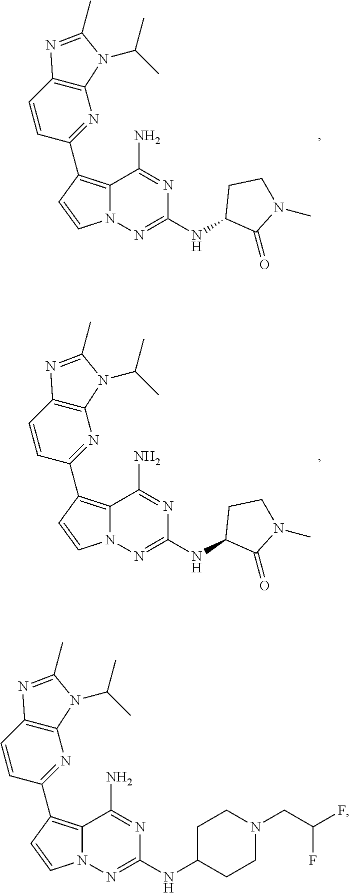 [SMILES: Cc1nc2ccc(-c3ccn4nc(N[C@@H]5CCN(C)C5=O)nc(N)c34)nc2n1C(C)C ; Cc1nc2ccc(-c3ccn4nc(N[C@H]5CCN(C)C5=O)nc(N)c34)nc2n1C(C)C ; Cc1nc2ccc(-c3ccn4nc(NC5CCN(CC(F)F)CC5)nc(N)c34)nc2n1C(C)C] 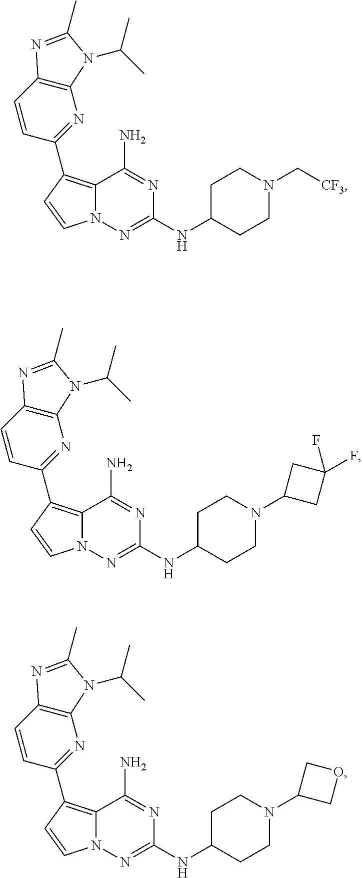 [SMILES: Cc1nc2ccc(-c3ccn4nc(NC5CCN(CC(F)(F)F)CC5)nc(N)c34)nc2n1C(C)C ; Cc1nc2ccc(-c3ccn4nc(NC5CCN(C6CC(F)(F)C6)CC5)nc(N)c34)nc2n1C(C)C ; Cc1nc2ccc(-c3ccn4nc(NC5CCN(C6COC6)CC5)nc(N)c34)nc2n1C(C)C] 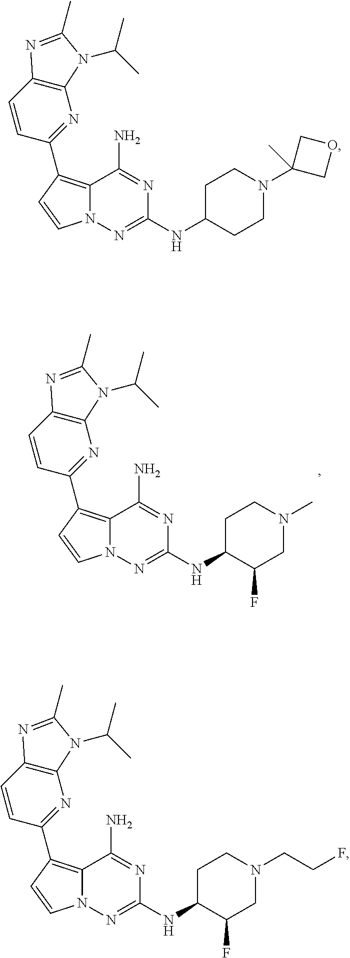 [SMILES: Cc1nc2ccc(-c3ccn4nc(NC5CCN(C6(C)COC6)CC5)nc(N)c34)nc2n1C(C)C ; Cc1nc2ccc(-c3ccn4nc(N[C@H]5CCN(C)C[C@H]5F)nc(N)c34)nc2n1C(C)C ; Cc1nc2ccc(-c3ccn4nc(N[C@H]5CCN(CCF)C[C@H]5F)nc(N)c34)nc2n1C(C)C] 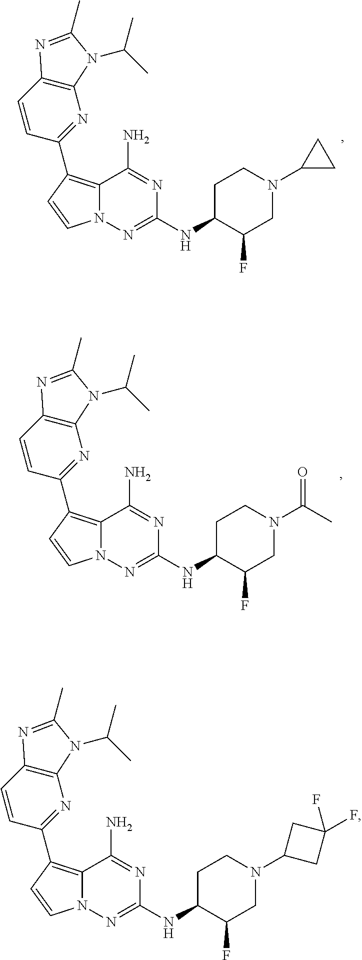 [SMILES: Cc1nc2ccc(-c3ccn4nc(N[C@H]5CCN(C6CC6)C[C@H]5F)nc(N)c34)nc2n1C(C)C ; CC(=O)N1CC[C@H](Nc2nc(N)c3c(-c4ccc5nc(C)n(C(C)C)c5n4)ccn3n2)[C@H](F)C1 ; Cc1nc2ccc(-c3ccn4nc(N[C@H]5CCN(C6CC(F)(F)C6)C[C@H]5F)nc(N)c34)nc2n1C(C)C] 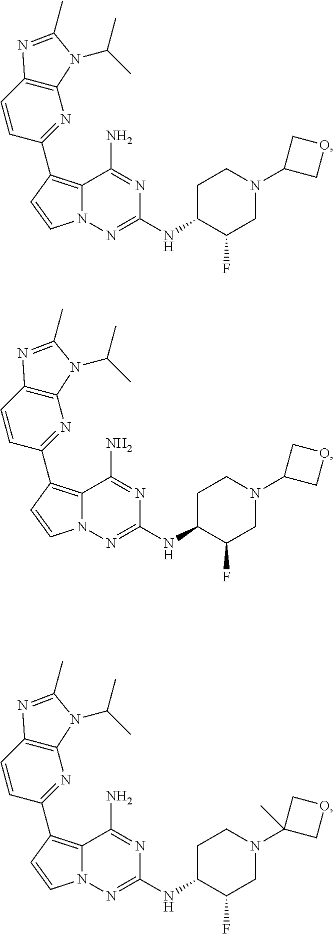 [SMILES: Cc1nc2ccc(-c3ccn4nc(N[C@@H]5CCN(C6COC6)C[C@@H]5F)nc(N)c34)nc2n1C(C)C ; Cc1nc2ccc(-c3ccn4nc(N[C@H]5CCN(C6COC6)C[C@H]5F)nc(N)c34)nc2n1C(C)C ; Cc1nc2ccc(-c3ccn4nc(N[C@@H]5CCN(C6(C)COC6)C[C@@H]5F)nc(N)c34)nc2n1C(C)C] 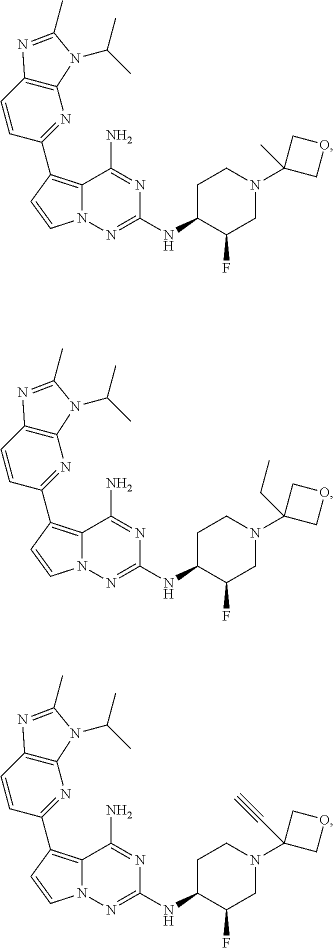 [SMILES: Cc1nc2ccc(-c3ccn4nc(N[C@H]5CCN(C6(C)COC6)C[C@H]5F)nc(N)c34)nc2n1C(C)C ; CCC1(N2CC[C@H](Nc3nc(N)c4c(-c5ccc6nc(C)n(C(C)C)c6n5)ccn4n3)[C@H](F)C2)COC1 ; C#CC1(N2CC[C@H](Nc3nc(N)c4c(-c5ccc6nc(C)n(C(C)C)c6n5)ccn4n3)[C@H](F)C2)COC1] 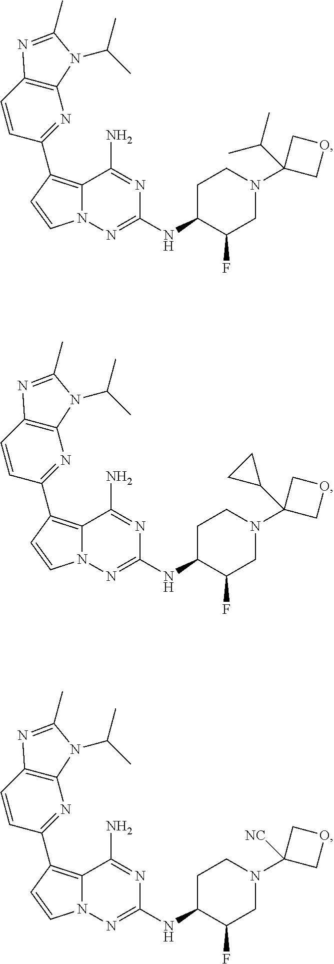 [SMILES: Cc1nc2ccc(-c3ccn4nc(N[C@H]5CCN(C6(C(C)C)COC6)C[C@H]5F)nc(N)c34)nc2n1C(C)C ; Cc1nc2ccc(-c3ccn4nc(N[C@H]5CCN(C6(C7CC7)COC6)C[C@H]5F)nc(N)c34)nc2n1C(C)C ; Cc1nc2ccc(-c3ccn4nc(N[C@H]5CCN(C6(C#N)COC6)C[C@H]5F)nc(N)c34)nc2n1C(C)C] 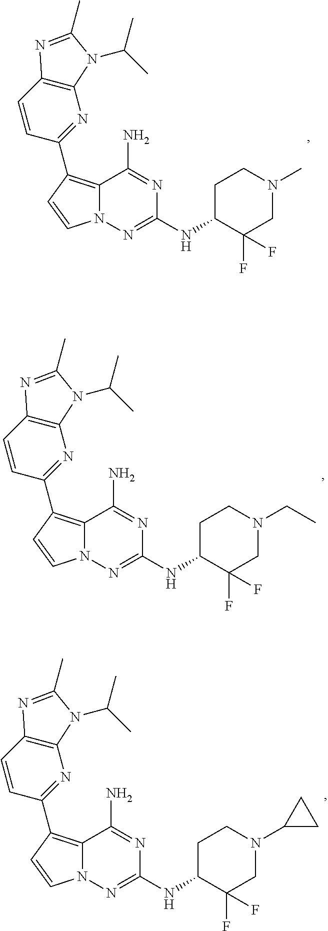 [SMILES: Cc1nc2ccc(-c3ccn4nc(N[C@@H]5CCN(C)CC5(F)F)nc(N)c34)nc2n1C(C)C ; CCN1CC[C@@H](Nc2nc(N)c3c(-c4ccc5nc(C)n(C(C)C)c5n4)ccn3n2)C(F)(F)C1 ; Cc1nc2ccc(-c3ccn4nc(N[C@@H]5CCN(C6CC6)CC5(F)F)nc(N)c34)nc2n1C(C)C] 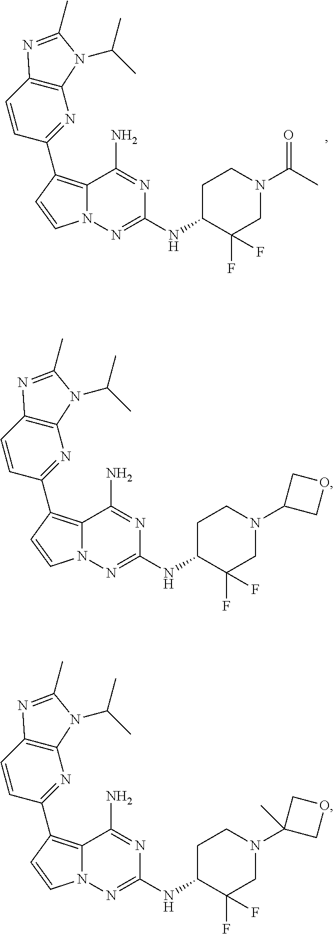 [SMILES: CC(=O)N1CC[C@@H](Nc2nc(N)c3c(-c4ccc5nc(C)n(C(C)C)c5n4)ccn3n2)C(F)(F)C1 ; Cc1nc2ccc(-c3ccn4nc(N[C@@H]5CCN(C6COC6)CC5(F)F)nc(N)c34)nc2n1C(C)C ; Cc1nc2ccc(-c3ccn4nc(N[C@@H]5CCN(C6(C)COC6)CC5(F)F)nc(N)c34)nc2n1C(C)C] 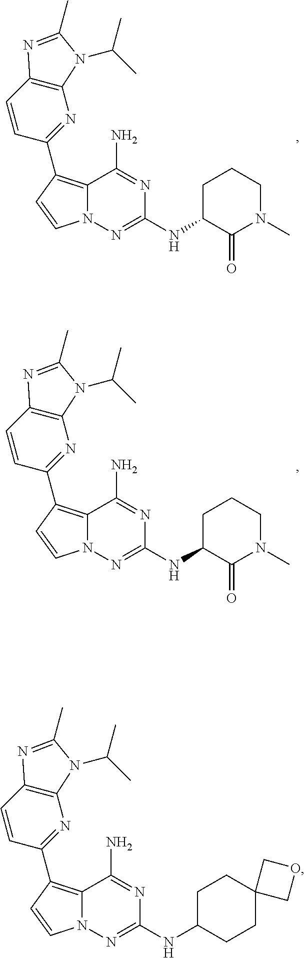 [SMILES: Cc1nc2ccc(-c3ccn4nc(N[C@@H]5CCCN(C)C5=O)nc(N)c34)nc2n1C(C)C ; Cc1nc2ccc(-c3ccn4nc(N[C@H]5CCCN(C)C5=O)nc(N)c34)nc2n1C(C)C ; Cc1nc2ccc(-c3ccn4nc(NC5CCC6(CC5)COC6)nc(N)c34)nc2n1C(C)C] 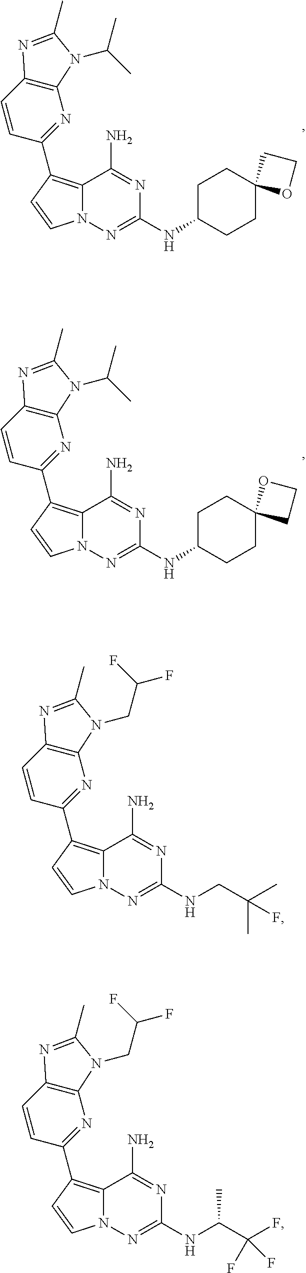 [SMILES: Cc1nc2ccc(-c3ccn4nc(N[C@H]5CC[C@@]6(CCO6)CC5)nc(N)c34)nc2n1C(C)C ; Cc1nc2ccc(-c3ccn4nc(N[C@H]5CC[C@]6(CCO6)CC5)nc(N)c34)nc2n1C(C)C ; Cc1nc2ccc(-c3ccn4nc(NCC(C)(C)F)nc(N)c34)nc2n1CC(F)F ; Cc1nc2ccc(-c3ccn4nc(N[C@H](C)C(F)(F)F)nc(N)c34)nc2n1CC(F)F] 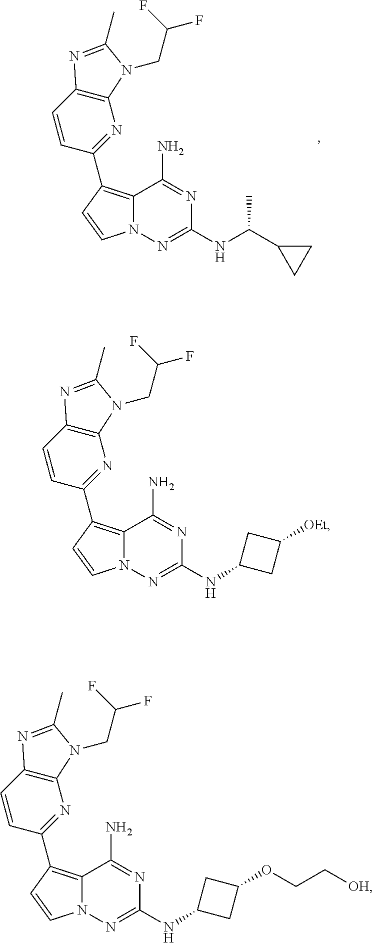 [SMILES: Cc1nc2ccc(-c3ccn4nc(N[C@H](C)C5CC5)nc(N)c34)nc2n1CC(F)F ; CCO[C@H]1C[C@@H](Nc2nc(N)c3c(-c4ccc5nc(C)n(CC(F)F)c5n4)ccn3n2)C1 ; Cc1nc2ccc(-c3ccn4nc(N[C@H]5C[C@@H](OCCO)C5)nc(N)c34)nc2n1CC(F)F] 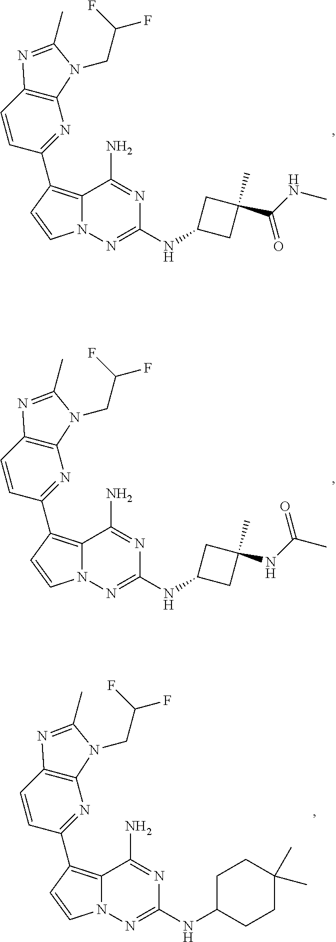 [SMILES: CNC(=O)[C@]1(C)C[C@@H](Nc2nc(N)c3c(-c4ccc5nc(C)n(CC(F)F)c5n4)ccn3n2)C1 ; CC(=O)N[C@]1(C)C[C@@H](Nc2nc(N)c3c(-c4ccc5nc(C)n(CC(F)F)c5n4)ccn3n2)C1 ; Cc1nc2ccc(-c3ccn4nc(NC5CCC(C)(C)CC5)nc(N)c34)nc2n1CC(F)F] 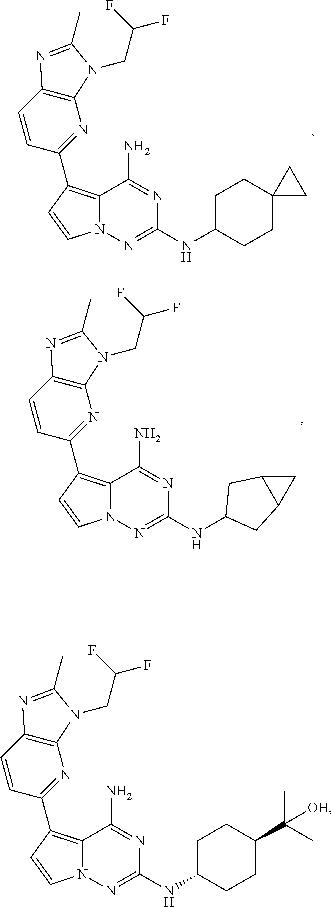 [SMILES: Cc1nc2ccc(-c3ccn4nc(NC5CCC6(CC5)CC6)nc(N)c34)nc2n1CC(F)F ; Cc1nc2ccc(-c3ccn4nc(NC5CC6CC6C5)nc(N)c34)nc2n1CC(F)F ; Cc1nc2ccc(-c3ccn4nc(N[C@H]5CC[C@H](C(C)(C)O)CC5)nc(N)c34)nc2n1CC(F)F] 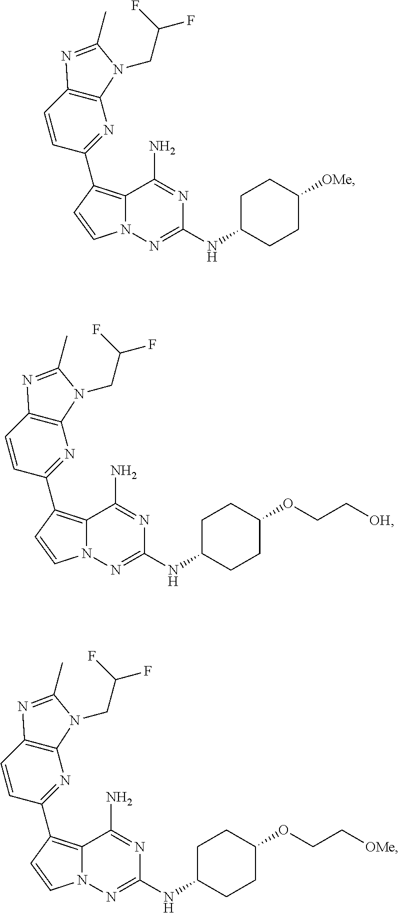 [SMILES: CO[C@H]1CC[C@@H](Nc2nc(N)c3c(-c4ccc5nc(C)n(CC(F)F)c5n4)ccn3n2)CC1 ; Cc1nc2ccc(-c3ccn4nc(N[C@H]5CC[C@@H](OCCO)CC5)nc(N)c34)nc2n1CC(F)F ; COCCO[C@H]1CC[C@@H](Nc2nc(N)c3c(-c4ccc5nc(C)n(CC(F)F)c5n4)ccn3n2)CC1] 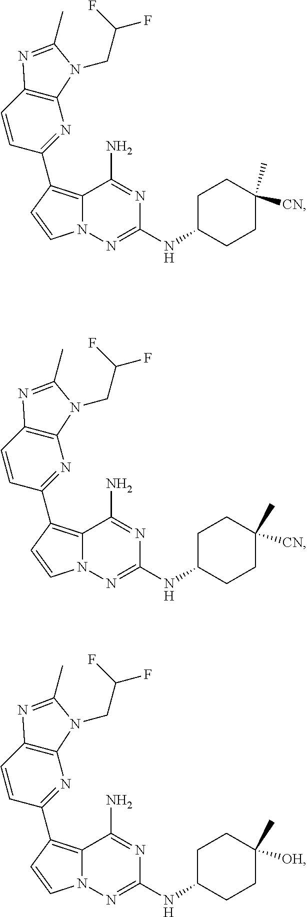 [SMILES: Cc1nc2ccc(-c3ccn4nc(N[C@H]5CC[C@](C)(C#N)CC5)nc(N)c34)nc2n1CC(F)F ; Cc1nc2ccc(-c3ccn4nc(N[C@H]5CC[C@@](C)(C#N)CC5)nc(N)c34)nc2n1CC(F)F ; Cc1nc2ccc(-c3ccn4nc(N[C@H]5CC[C@@](C)(O)CC5)nc(N)c34)nc2n1CC(F)F] 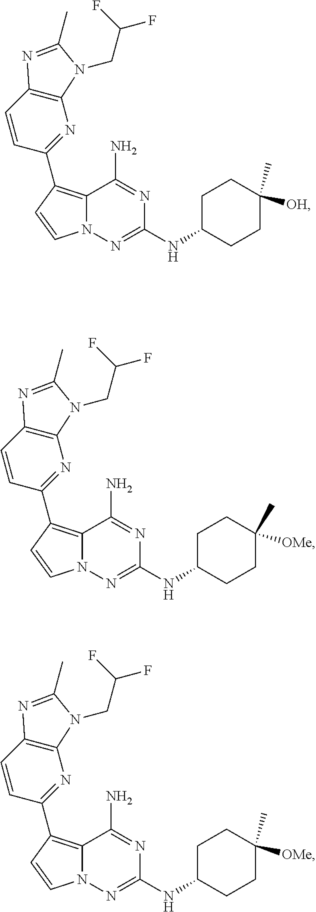 [SMILES: Cc1nc2ccc(-c3ccn4nc(N[C@H]5CC[C@](C)(O)CC5)nc(N)c34)nc2n1CC(F)F ; CO[C@]1(C)CC[C@H](Nc2nc(N)c3c(-c4ccc5nc(C)n(CC(F)F)c5n4)ccn3n2)CC1 ; CO[C@]1(C)CC[C@@H](Nc2nc(N)c3c(-c4ccc5nc(C)n(CC(F)F)c5n4)ccn3n2)CC1] 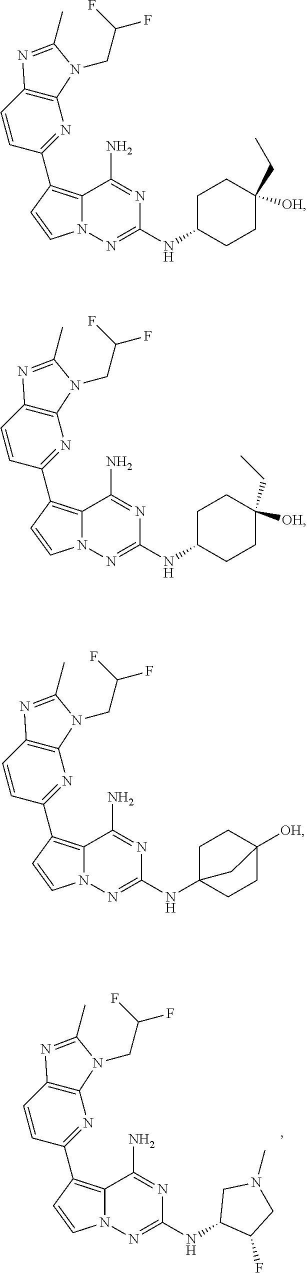 [SMILES: CC[C@]1(O)CC[C@@H](Nc2nc(N)c3c(-c4ccc5nc(C)n(CC(F)F)c5n4)ccn3n2)CC1 ; CC[C@]1(O)CC[C@H](Nc2nc(N)c3c(-c4ccc5nc(C)n(CC(F)F)c5n4)ccn3n2)CC1 ; Cc1nc2ccc(-c3ccn4nc(NC56CCC(O)(CC5)C6)nc(N)c34)nc2n1CC(F)F ; Cc1nc2ccc(-c3ccn4nc(N[C@@H]5CN(C)C[C@@H]5F)nc(N)c34)nc2n1CC(F)F] 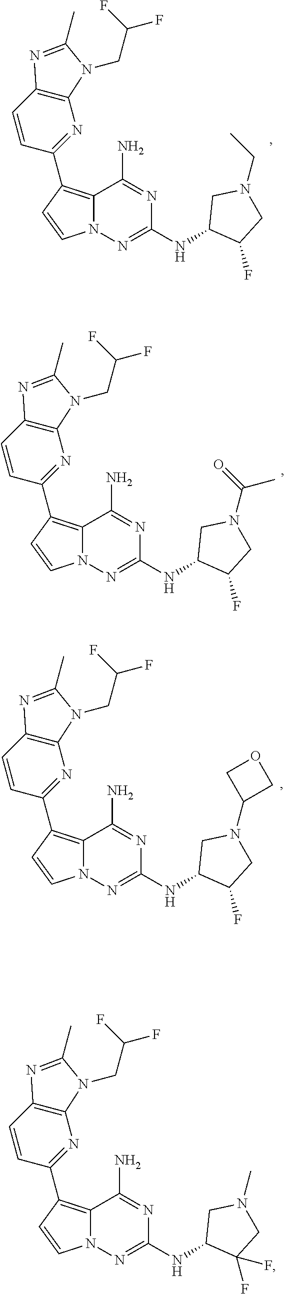 [SMILES: CCN1C[C@H](F)[C@H](Nc2nc(N)c3c(-c4ccc5nc(C)n(CC(F)F)c5n4)ccn3n2)C1 ; CC(=O)N1C[C@H](F)[C@H](Nc2nc(N)c3c(-c4ccc5nc(C)n(CC(F)F)c5n4)ccn3n2)C1 ; Cc1nc2ccc(-c3ccn4nc(N[C@@H]5CN(C6COC6)C[C@@H]5F)nc(N)c34)nc2n1CC(F)F ; Cc1nc2ccc(-c3ccn4nc(N[C@@H]5CN(C)CC5(F)F)nc(N)c34)nc2n1CC(F)F] 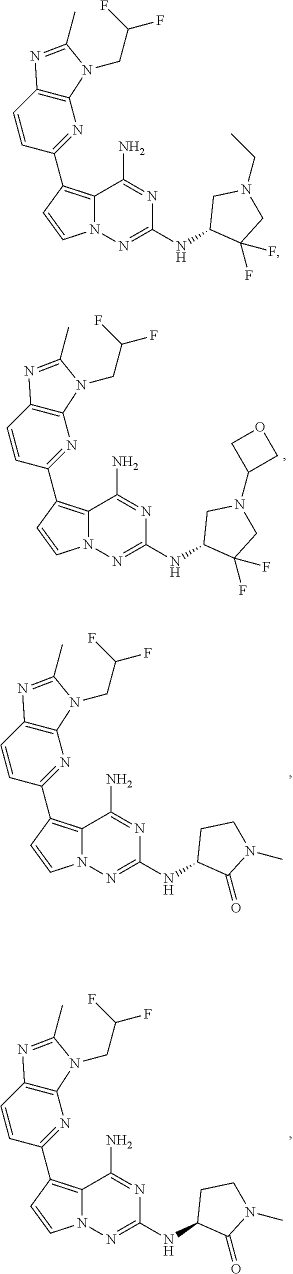 [SMILES: CCN1C[C@@H](Nc2nc(N)c3c(-c4ccc5nc(C)n(CC(F)F)c5n4)ccn3n2)C(F)(F)C1 ; Cc1nc2ccc(-c3ccn4nc(N[C@@H]5CN(C6COC6)CC5(F)F)nc(N)c34)nc2n1CC(F)F ; Cc1nc2ccc(-c3ccn4nc(N[C@@H]5CCN(C)C5=O)nc(N)c34)nc2n1CC(F)F ; Cc1nc2ccc(-c3ccn4nc(N[C@H]5CCN(C)C5=O)nc(N)c34)nc2n1CC(F)F] 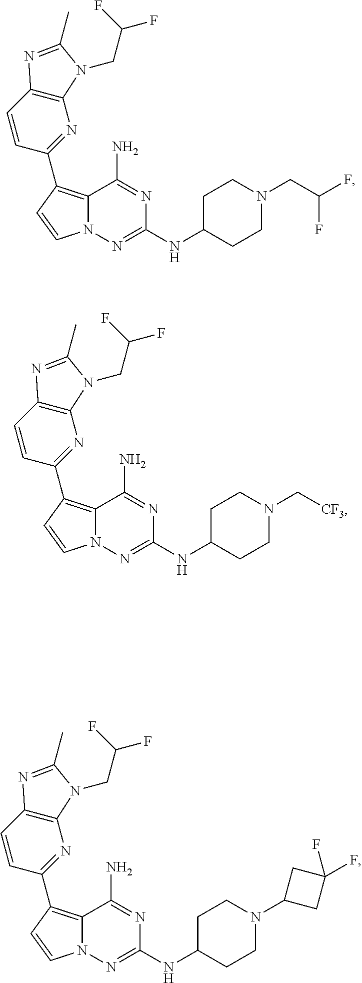 [SMILES: Cc1nc2ccc(-c3ccn4nc(NC5CCN(CC(F)F)CC5)nc(N)c34)nc2n1CC(F)F ; Cc1nc2ccc(-c3ccn4nc(NC5CCN(CC(F)(F)F)CC5)nc(N)c34)nc2n1CC(F)F ; Cc1nc2ccc(-c3ccn4nc(NC5CCN(C6CC(F)(F)C6)CC5)nc(N)c34)nc2n1CC(F)F] 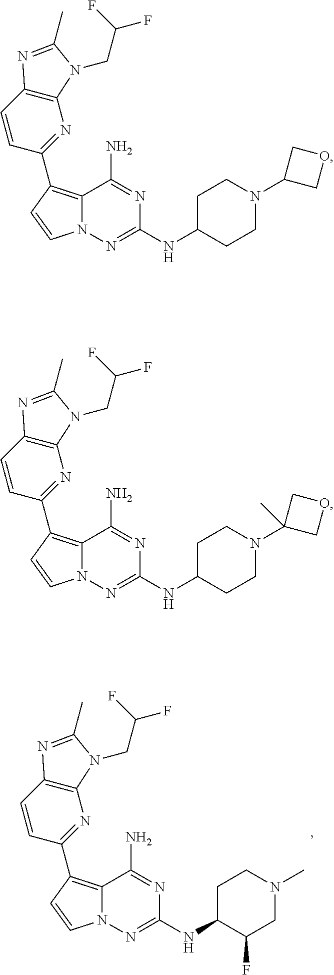 [SMILES: Cc1nc2ccc(-c3ccn4nc(NC5CCN(C6COC6)CC5)nc(N)c34)nc2n1CC(F)F ; Cc1nc2ccc(-c3ccn4nc(NC5CCN(C6(C)COC6)CC5)nc(N)c34)nc2n1CC(F)F ; Cc1nc2ccc(-c3ccn4nc(N[C@H]5CCN(C)C[C@H]5F)nc(N)c34)nc2n1CC(F)F] 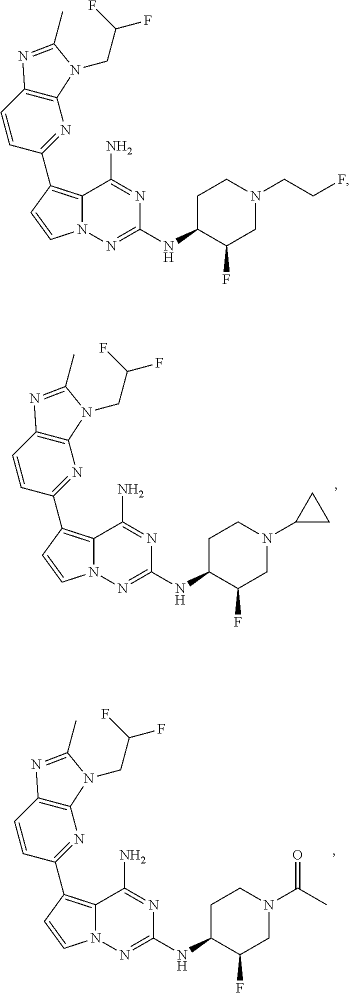 [SMILES: Cc1nc2ccc(-c3ccn4nc(N[C@H]5CCN(CCF)C[C@H]5F)nc(N)c34)nc2n1CC(F)F ; Cc1nc2ccc(-c3ccn4nc(N[C@H]5CCN(C6CC6)C[C@H]5F)nc(N)c34)nc2n1CC(F)F ; CC(=O)N1CC[C@H](Nc2nc(N)c3c(-c4ccc5nc(C)n(CC(F)F)c5n4)ccn3n2)[C@H](F)C1] 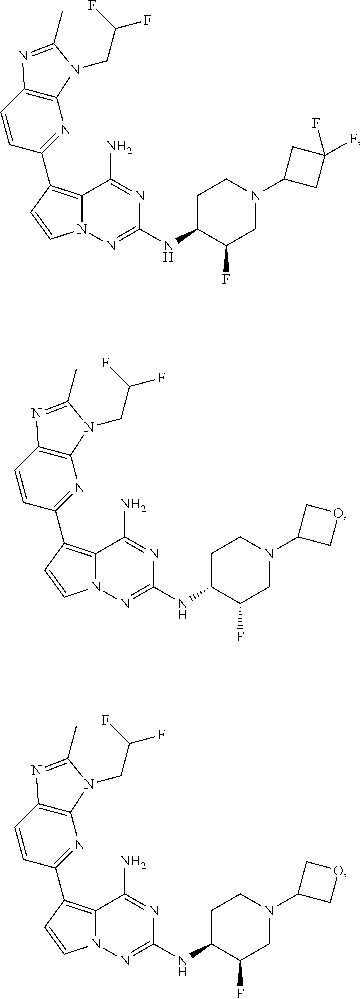 [SMILES: Cc1nc2ccc(-c3ccn4nc(N[C@H]5CCN(C6CC(F)(F)C6)C[C@H]5F)nc(N)c34)nc2n1CC(F)F ; Cc1nc2ccc(-c3ccn4nc(N[C@@H]5CCN(C6COC6)C[C@@H]5F)nc(N)c34)nc2n1CC(F)F ; Cc1nc2ccc(-c3ccn4nc(N[C@H]5CCN(C6COC6)C[C@H]5F)nc(N)c34)nc2n1CC(F)F] 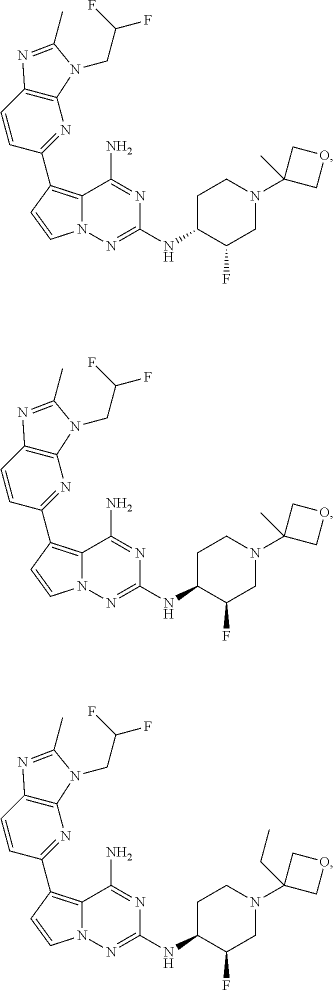 [SMILES: Cc1nc2ccc(-c3ccn4nc(N[C@@H]5CCN(C6(C)COC6)C[C@@H]5F)nc(N)c34)nc2n1CC(F)F ; Cc1nc2ccc(-c3ccn4nc(N[C@H]5CCN(C6(C)COC6)C[C@H]5F)nc(N)c34)nc2n1CC(F)F ; CCC1(N2CC[C@H](Nc3nc(N)c4c(-c5ccc6nc(C)n(CC(F)F)c6n5)ccn4n3)[C@H](F)C2)COC1] 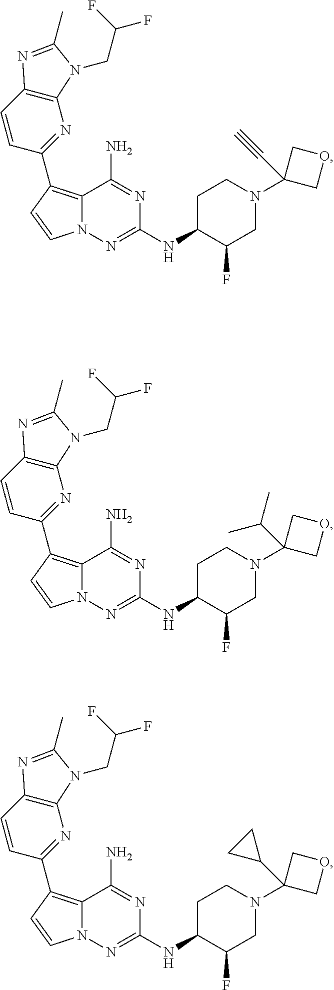 [SMILES: C#CC1(N2CC[C@H](Nc3nc(N)c4c(-c5ccc6nc(C)n(CC(F)F)c6n5)ccn4n3)[C@H](F)C2)COC1 ; Cc1nc2ccc(-c3ccn4nc(N[C@H]5CCN(C6(C(C)C)COC6)C[C@H]5F)nc(N)c34)nc2n1CC(F)F ; Cc1nc2ccc(-c3ccn4nc(N[C@H]5CCN(C6(C7CC7)COC6)C[C@H]5F)nc(N)c34)nc2n1CC(F)F] 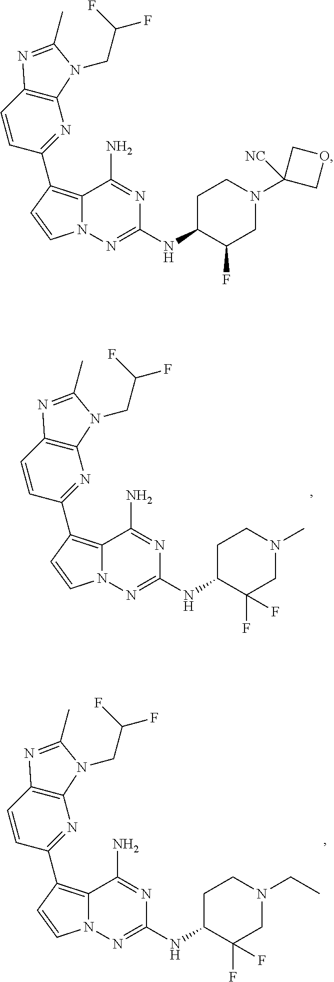 [SMILES: Cc1nc2ccc(-c3ccn4nc(N[C@H]5CCN(C6(C#N)COC6)C[C@H]5F)nc(N)c34)nc2n1CC(F)F ; Cc1nc2ccc(-c3ccn4nc(N[C@@H]5CCN(C)CC5(F)F)nc(N)c34)nc2n1CC(F)F ; CCN1CC[C@@H](Nc2nc(N)c3c(-c4ccc5nc(C)n(CC(F)F)c5n4)ccn3n2)C(F)(F)C1] 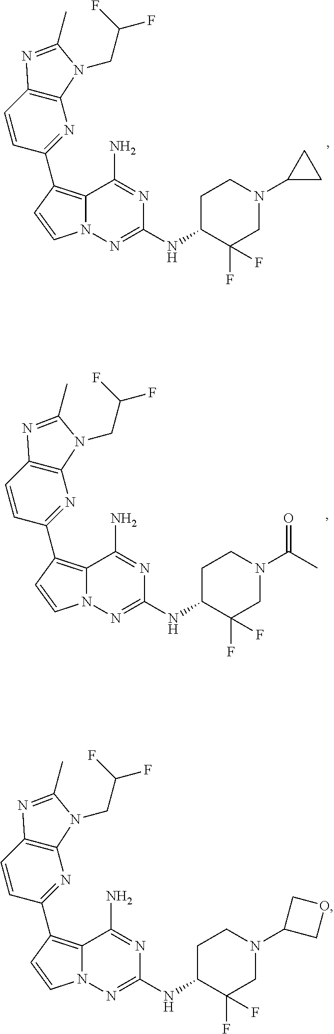 [SMILES: Cc1nc2ccc(-c3ccn4nc(N[C@@H]5CCN(C6CC6)CC5(F)F)nc(N)c34)nc2n1CC(F)F ; CC(=O)N1CC[C@@H](Nc2nc(N)c3c(-c4ccc5nc(C)n(CC(F)F)c5n4)ccn3n2)C(F)(F)C1 ; Cc1nc2ccc(-c3ccn4nc(N[C@@H]5CCN(C6COC6)CC5(F)F)nc(N)c34)nc2n1CC(F)F] 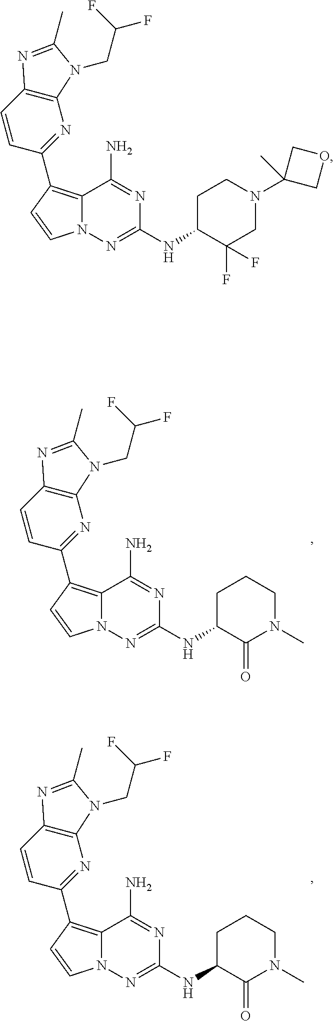 [SMILES: Cc1nc2ccc(-c3ccn4nc(N[C@@H]5CCN(C6(C)COC6)CC5(F)F)nc(N)c34)nc2n1CC(F)F ; Cc1nc2ccc(-c3ccn4nc(N[C@@H]5CCCN(C)C5=O)nc(N)c34)nc2n1CC(F)F ; Cc1nc2ccc(-c3ccn4nc(N[C@H]5CCCN(C)C5=O)nc(N)c34)nc2n1CC(F)F] 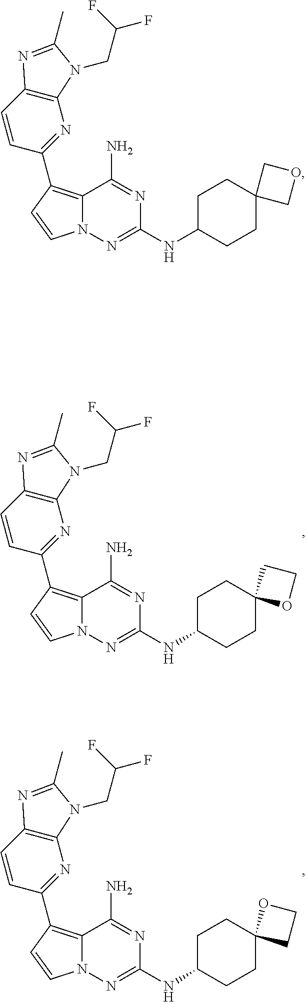 [SMILES: Cc1nc2ccc(-c3ccn4nc(NC5CCC6(CC5)COC6)nc(N)c34)nc2n1CC(F)F ; Cc1nc2ccc(-c3ccn4nc(N[C@H]5CC[C@@]6(CCO6)CC5)nc(N)c34)nc2n1CC(F)F ; Cc1nc2ccc(-c3ccn4nc(N[C@H]5CC[C@]6(CCO6)CC5)nc(N)c34)nc2n1CC(F)F] 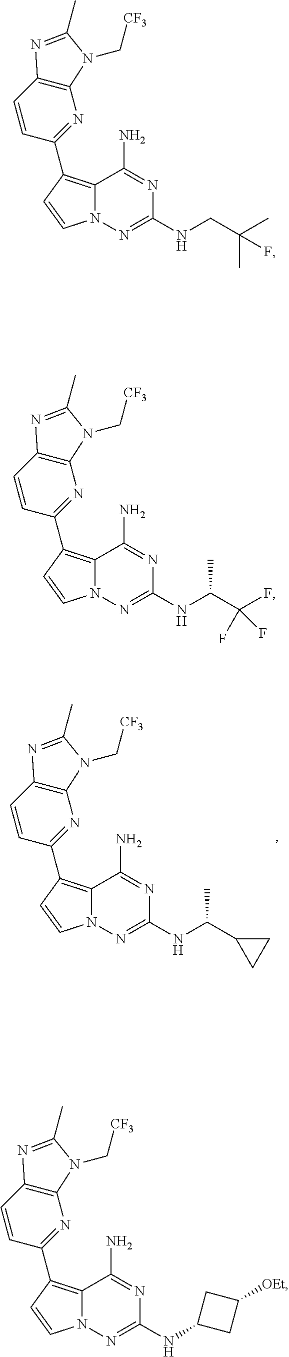 [SMILES: Cc1nc2ccc(-c3ccn4nc(NCC(C)(C)F)nc(N)c34)nc2n1CC(F)(F)F ; Cc1nc2ccc(-c3ccn4nc(N[C@H](C)C(F)(F)F)nc(N)c34)nc2n1CC(F)(F)F ; Cc1nc2ccc(-c3ccn4nc(N[C@H](C)C5CC5)nc(N)c34)nc2n1CC(F)(F)F ; CCO[C@H]1C[C@@H](Nc2nc(N)c3c(-c4ccc5nc(C)n(CC(F)(F)F)c5n4)ccn3n2)C1] 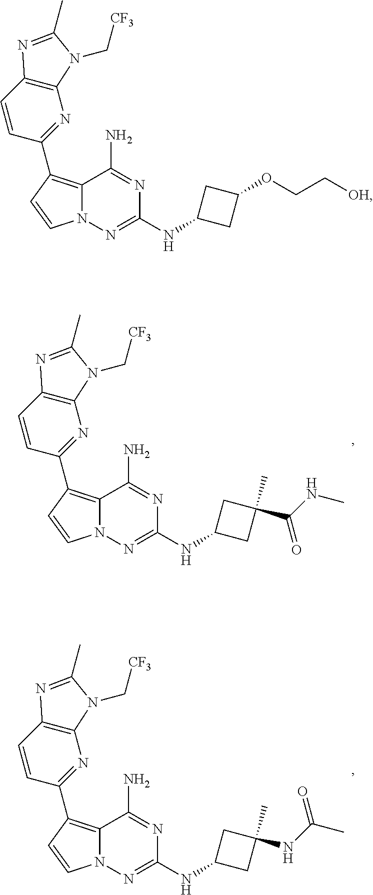 [SMILES: Cc1nc2ccc(-c3ccn4nc(N[C@H]5C[C@@H](OCCO)C5)nc(N)c34)nc2n1CC(F)(F)F ; CNC(=O)[C@]1(C)C[C@@H](Nc2nc(N)c3c(-c4ccc5nc(C)n(CC(F)(F)F)c5n4)ccn3n2)C1 ; CC(=O)N[C@]1(C)C[C@@H](Nc2nc(N)c3c(-c4ccc5nc(C)n(CC(F)(F)F)c5n4)ccn3n2)C1] 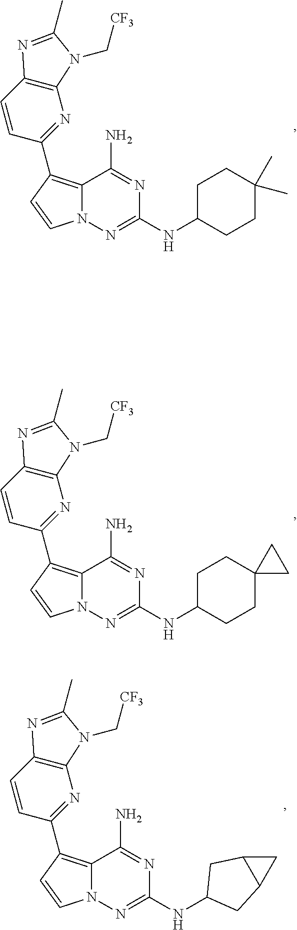 [SMILES: Cc1nc2ccc(-c3ccn4nc(NC5CCC(C)(C)CC5)nc(N)c34)nc2n1CC(F)(F)F ; Cc1nc2ccc(-c3ccn4nc(NC5CCC6(CC5)CC6)nc(N)c34)nc2n1CC(F)(F)F ; Cc1nc2ccc(-c3ccn4nc(NC5CC6CC6C5)nc(N)c34)nc2n1CC(F)(F)F] 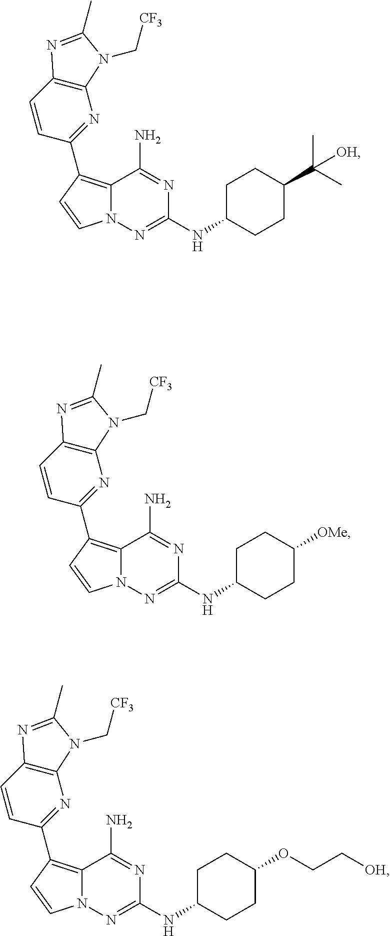 [SMILES: Cc1nc2ccc(-c3ccn4nc(N[C@H]5CC[C@H](C(C)(C)O)CC5)nc(N)c34)nc2n1CC(F)(F)F ; CO[C@H]1CC[C@@H](Nc2nc(N)c3c(-c4ccc5nc(C)n(CC(F)(F)F)c5n4)ccn3n2)CC1 ; Cc1nc2ccc(-c3ccn4nc(N[C@H]5CC[C@@H](OCCO)CC5)nc(N)c34)nc2n1CC(F)(F)F] 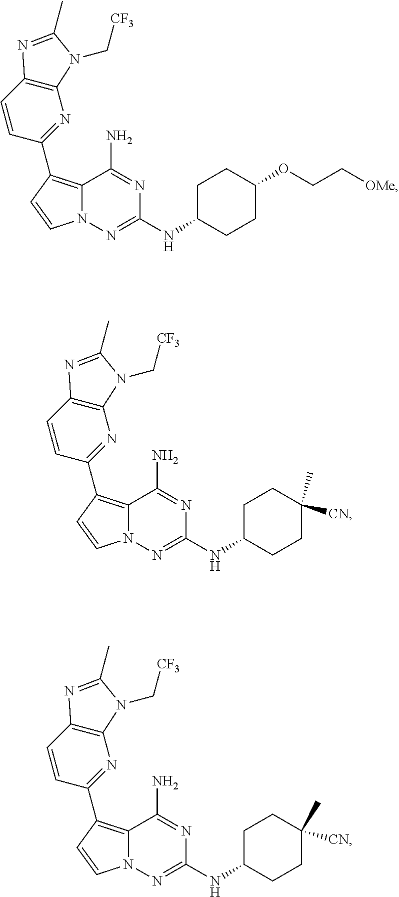 [SMILES: COCCO[C@H]1CC[C@@H](Nc2nc(N)c3c(-c4ccc5nc(C)n(CC(F)(F)F)c5n4)ccn3n2)CC1 ; Cc1nc2ccc(-c3ccn4nc(N[C@H]5CC[C@](C)(C#N)CC5)nc(N)c34)nc2n1CC(F)(F)F ; Cc1nc2ccc(-c3ccn4nc(N[C@H]5CC[C@@](C)(C#N)CC5)nc(N)c34)nc2n1CC(F)(F)F] 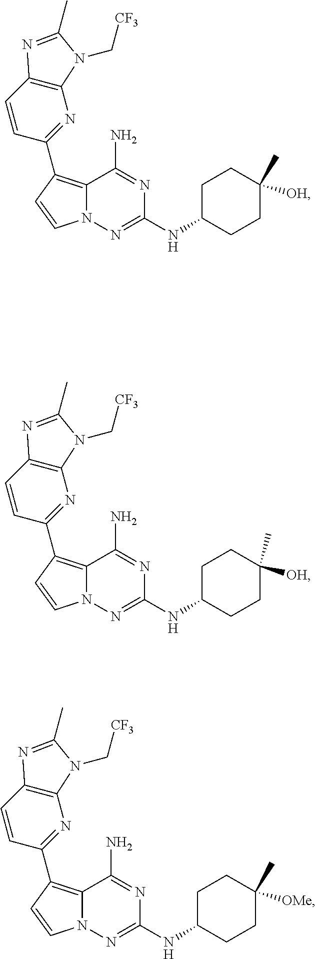 [SMILES: Cc1nc2ccc(-c3ccn4nc(N[C@H]5CC[C@@](C)(O)CC5)nc(N)c34)nc2n1CC(F)(F)F ; Cc1nc2ccc(-c3ccn4nc(N[C@H]5CC[C@](C)(O)CC5)nc(N)c34)nc2n1CC(F)(F)F ; CO[C@]1(C)CC[C@H](Nc2nc(N)c3c(-c4ccc5nc(C)n(CC(F)(F)F)c5n4)ccn3n2)CC1] 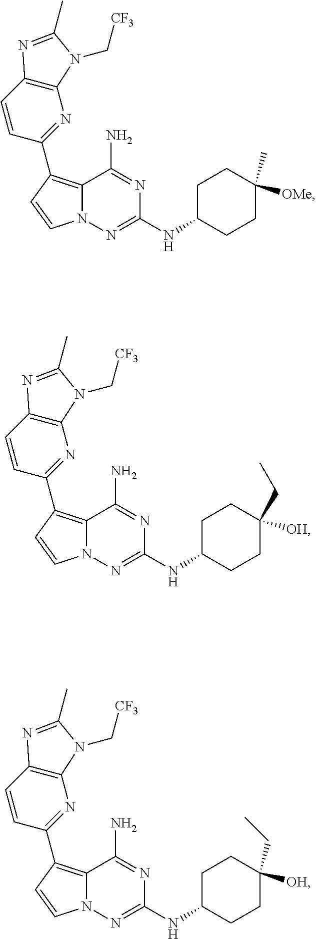 [SMILES: CO[C@]1(C)CC[C@@H](Nc2nc(N)c3c(-c4ccc5nc(C)n(CC(F)(F)F)c5n4)ccn3n2)CC1 ; CC[C@]1(O)CC[C@@H](Nc2nc(N)c3c(-c4ccc5nc(C)n(CC(F)(F)F)c5n4)ccn3n2)CC1 ; CC[C@]1(O)CC[C@H](Nc2nc(N)c3c(-c4ccc5nc(C)n(CC(F)(F)F)c5n4)ccn3n2)CC1] 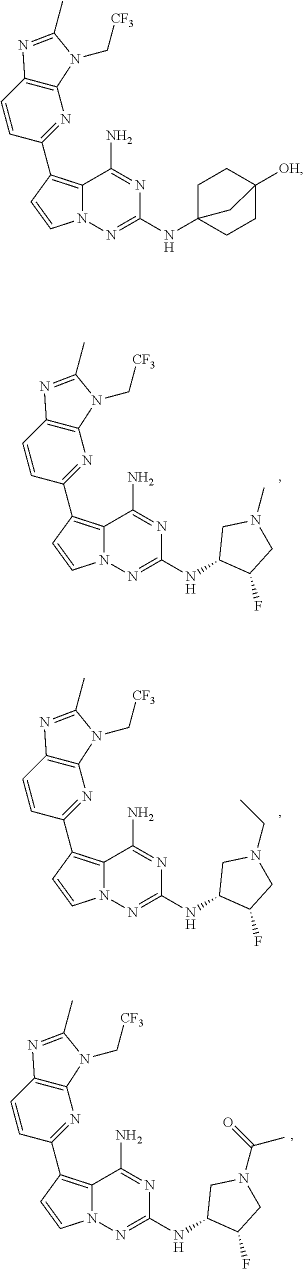 [SMILES: Cc1nc2ccc(-c3ccn4nc(NC56CCC(O)(CC5)C6)nc(N)c34)nc2n1CC(F)(F)F ; Cc1nc2ccc(-c3ccn4nc(N[C@@H]5CN(C)C[C@@H]5F)nc(N)c34)nc2n1CC(F)(F)F ; CCN1C[C@H](F)[C@H](Nc2nc(N)c3c(-c4ccc5nc(C)n(CC(F)(F)F)c5n4)ccn3n2)C1 ; CC(=O)N1C[C@H](F)[C@H](Nc2nc(N)c3c(-c4ccc5nc(C)n(CC(F)(F)F)c5n4)ccn3n2)C1] 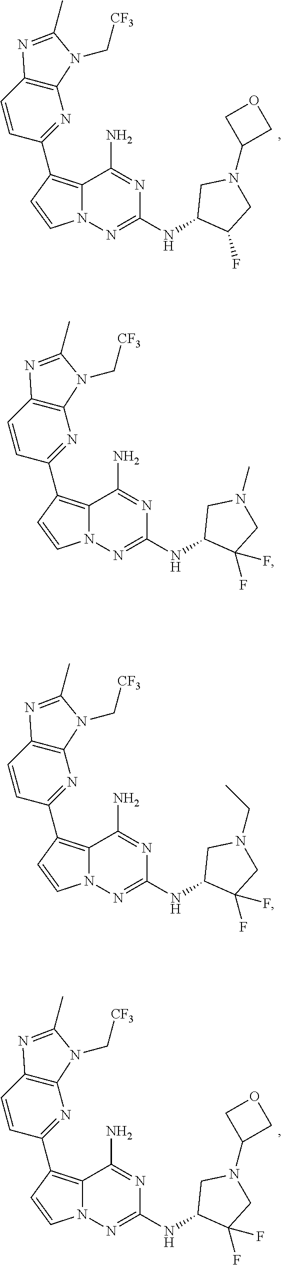 [SMILES: Cc1nc2ccc(-c3ccn4nc(N[C@@H]5CN(C6COC6)C[C@@H]5F)nc(N)c34)nc2n1CC(F)(F)F ; Cc1nc2ccc(-c3ccn4nc(N[C@@H]5CN(C)CC5(F)F)nc(N)c34)nc2n1CC(F)(F)F ; CCN1C[C@@H](Nc2nc(N)c3c(-c4ccc5nc(C)n(CC(F)(F)F)c5n4)ccn3n2)C(F)(F)C1 ; Cc1nc2ccc(-c3ccn4nc(N[C@@H]5CN(C6COC6)CC5(F)F)nc(N)c34)nc2n1CC(F)(F)F] 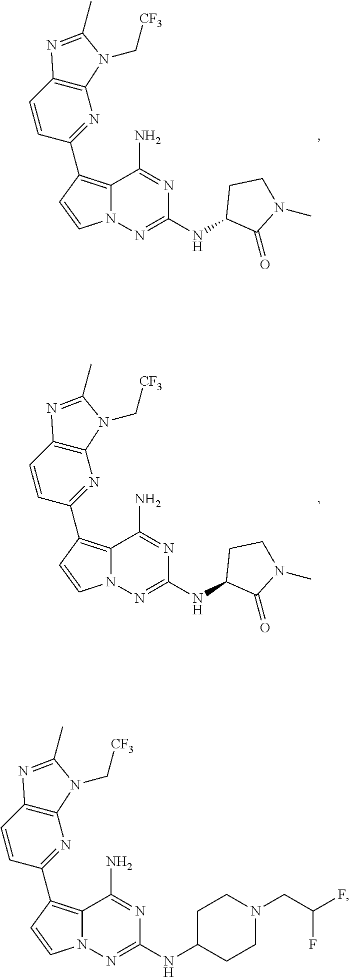 [SMILES: Cc1nc2ccc(-c3ccn4nc(N[C@@H]5CCN(C)C5=O)nc(N)c34)nc2n1CC(F)(F)F ; Cc1nc2ccc(-c3ccn4nc(N[C@H]5CCN(C)C5=O)nc(N)c34)nc2n1CC(F)(F)F ; Cc1nc2ccc(-c3ccn4nc(NC5CCN(CC(F)F)CC5)nc(N)c34)nc2n1CC(F)(F)F] 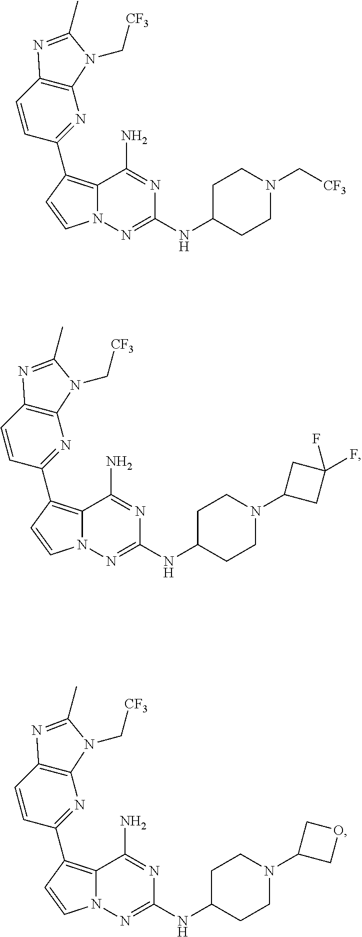 [SMILES: Cc1nc2ccc(-c3ccn4nc(NC5CCN(CC(F)(F)F)CC5)nc(N)c34)nc2n1CC(F)(F)F ; Cc1nc2ccc(-c3ccn4nc(NC5CCN(C6CC(F)(F)C6)CC5)nc(N)c34)nc2n1CC(F)(F)F ; Cc1nc2ccc(-c3ccn4nc(NC5CCN(C6COC6)CC5)nc(N)c34)nc2n1CC(F)(F)F] 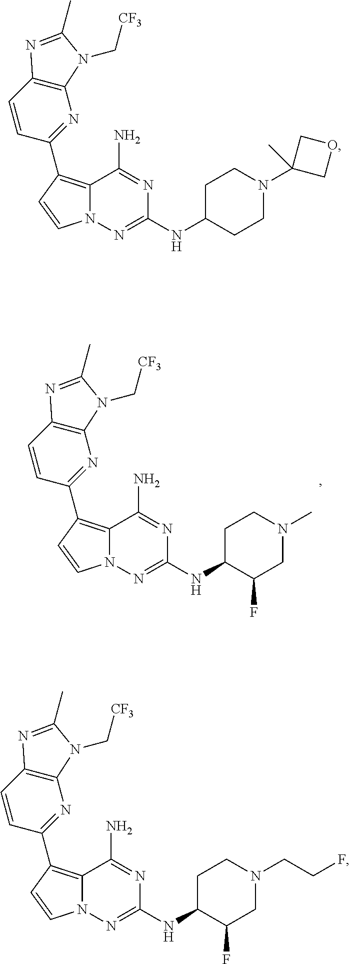 [SMILES: Cc1nc2ccc(-c3ccn4nc(NC5CCN(C6(C)COC6)CC5)nc(N)c34)nc2n1CC(F)(F)F ; Cc1nc2ccc(-c3ccn4nc(N[C@H]5CCN(C)C[C@H]5F)nc(N)c34)nc2n1CC(F)(F)F ; Cc1nc2ccc(-c3ccn4nc(N[C@H]5CCN(CCF)C[C@H]5F)nc(N)c34)nc2n1CC(F)(F)F] 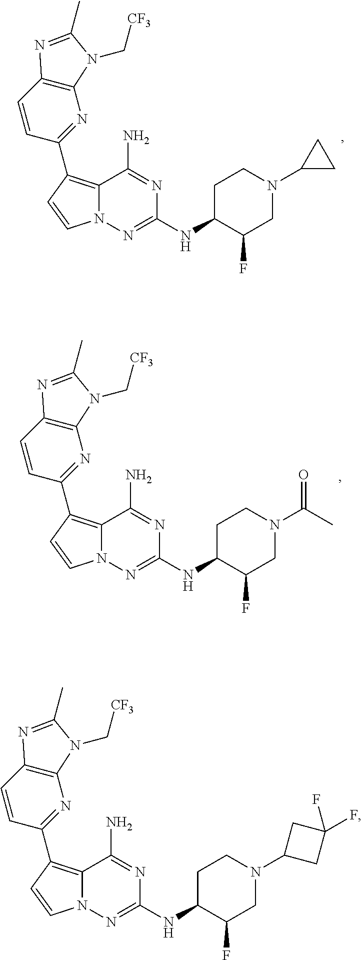 [SMILES: Cc1nc2ccc(-c3ccn4nc(N[C@H]5CCN(C6CC6)C[C@H]5F)nc(N)c34)nc2n1CC(F)(F)F ; CC(=O)N1CC[C@H](Nc2nc(N)c3c(-c4ccc5nc(C)n(CC(F)(F)F)c5n4)ccn3n2)[C@H](F)C1 ; Cc1nc2ccc(-c3ccn4nc(N[C@H]5CCN(C6CC(F)(F)C6)C[C@H]5F)nc(N)c34)nc2n1CC(F)(F)F] 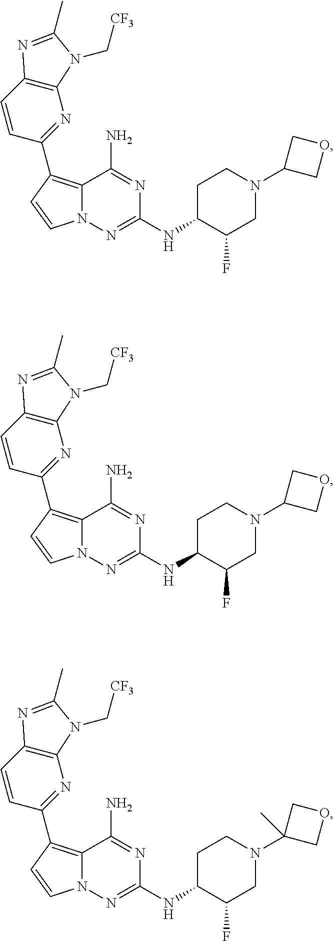 [SMILES: Cc1nc2ccc(-c3ccn4nc(N[C@@H]5CCN(C6COC6)C[C@@H]5F)nc(N)c34)nc2n1CC(F)(F)F ; Cc1nc2ccc(-c3ccn4nc(N[C@H]5CCN(C6COC6)C[C@H]5F)nc(N)c34)nc2n1CC(F)(F)F ; Cc1nc2ccc(-c3ccn4nc(N[C@@H]5CCN(C6(C)COC6)C[C@@H]5F)nc(N)c34)nc2n1CC(F)(F)F] 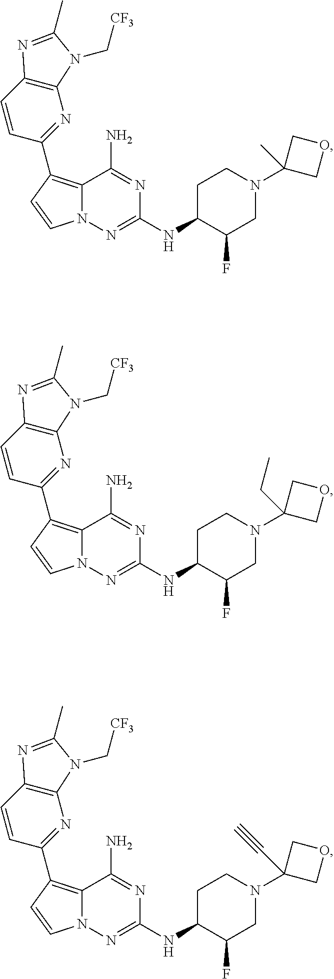 [SMILES: Cc1nc2ccc(-c3ccn4nc(N[C@H]5CCN(C6(C)COC6)C[C@H]5F)nc(N)c34)nc2n1CC(F)(F)F ; CCC1(N2CC[C@H](Nc3nc(N)c4c(-c5ccc6nc(C)n(CC(F)(F)F)c6n5)ccn4n3)[C@H](F)C2)COC1 ; C#CC1(N2CC[C@H](Nc3nc(N)c4c(-c5ccc6nc(C)n(CC(F)(F)F)c6n5)ccn4n3)[C@H](F)C2)COC1] 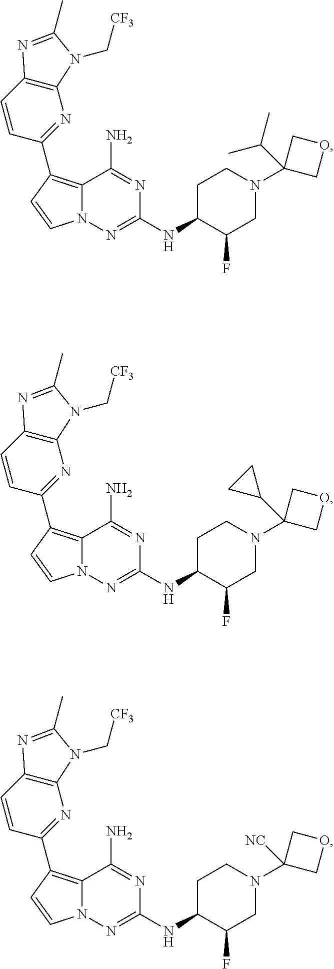 [SMILES: Cc1nc2ccc(-c3ccn4nc(N[C@H]5CCN(C6(C(C)C)COC6)C[C@H]5F)nc(N)c34)nc2n1CC(F)(F)F ; Cc1nc2ccc(-c3ccn4nc(N[C@H]5CCN(C6(C7CC7)COC6)C[C@H]5F)nc(N)c34)nc2n1CC(F)(F)F ; Cc1nc2ccc(-c3ccn4nc(N[C@H]5CCN(C6(C#N)COC6)C[C@H]5F)nc(N)c34)nc2n1CC(F)(F)F] 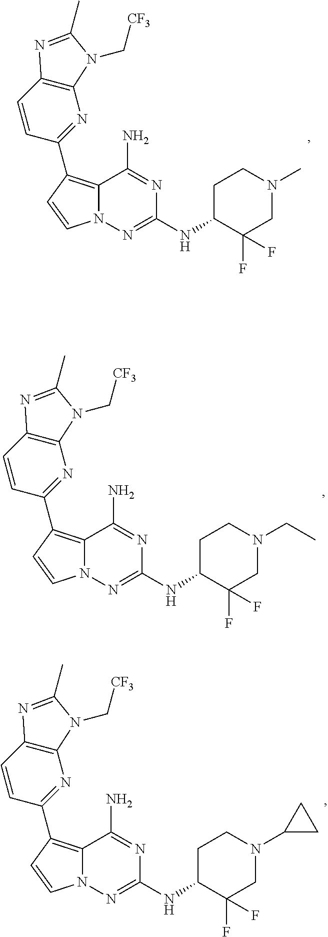 [SMILES: Cc1nc2ccc(-c3ccn4nc(N[C@@H]5CCN(C)CC5(F)F)nc(N)c34)nc2n1CC(F)(F)F ; CCN1CC[C@@H](Nc2nc(N)c3c(-c4ccc5nc(C)n(CC(F)(F)F)c5n4)ccn3n2)C(F)(F)C1 ; Cc1nc2ccc(-c3ccn4nc(N[C@@H]5CCN(C6CC6)CC5(F)F)nc(N)c34)nc2n1CC(F)(F)F] 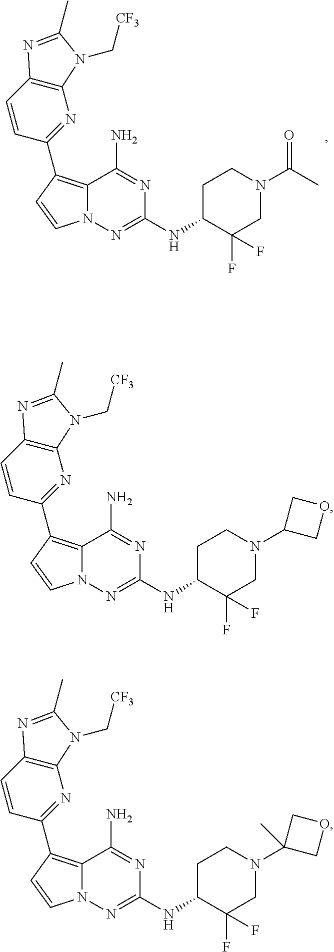 [SMILES: CC(=O)N1CC[C@@H](Nc2nc(N)c3c(-c4ccc5nc(C)n(CC(F)(F)F)c5n4)ccn3n2)C(F)(F)C1 ; Cc1nc2ccc(-c3ccn4nc(N[C@@H]5CCN(C6COC6)CC5(F)F)nc(N)c34)nc2n1CC(F)(F)F ; Cc1nc2ccc(-c3ccn4nc(N[C@@H]5CCN(C6(C)COC6)CC5(F)F)nc(N)c34)nc2n1CC(F)(F)F] 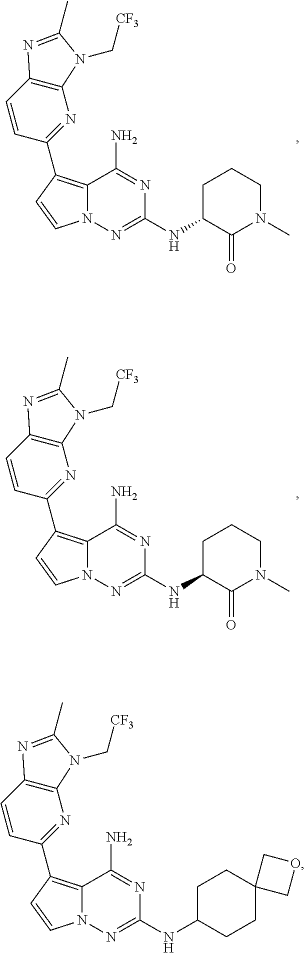 [SMILES: Cc1nc2ccc(-c3ccn4nc(N[C@@H]5CCCN(C)C5=O)nc(N)c34)nc2n1CC(F)(F)F ; Cc1nc2ccc(-c3ccn4nc(N[C@H]5CCCN(C)C5=O)nc(N)c34)nc2n1CC(F)(F)F ; Cc1nc2ccc(-c3ccn4nc(NC5CCC6(CC5)COC6)nc(N)c34)nc2n1CC(F)(F)F] 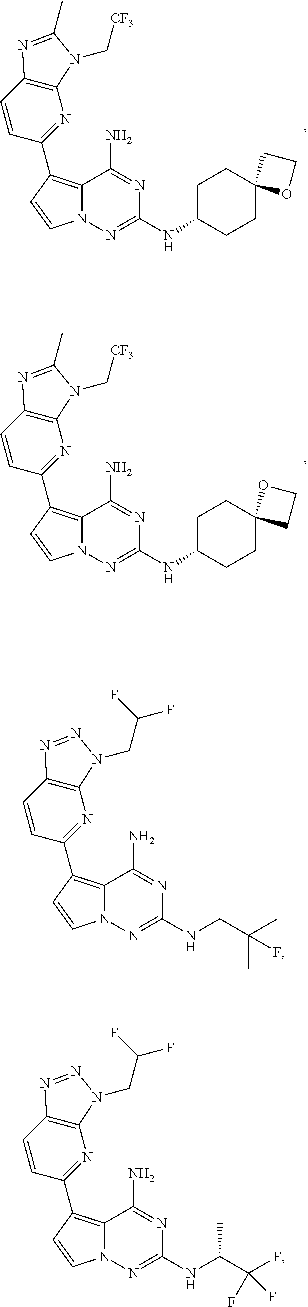 [SMILES: Cc1nc2ccc(-c3ccn4nc(N[C@H]5CC[C@@]6(CCO6)CC5)nc(N)c34)nc2n1CC(F)(F)F ; Cc1nc2ccc(-c3ccn4nc(N[C@H]5CC[C@]6(CCO6)CC5)nc(N)c34)nc2n1CC(F)(F)F ; CC(C)(F)CNc1nc(N)c2c(-c3ccc4nnn(CC(F)F)c4n3)ccn2n1 ; C[C@@H](Nc1nc(N)c2c(-c3ccc4nnn(CC(F)F)c4n3)ccn2n1)C(F)(F)F] 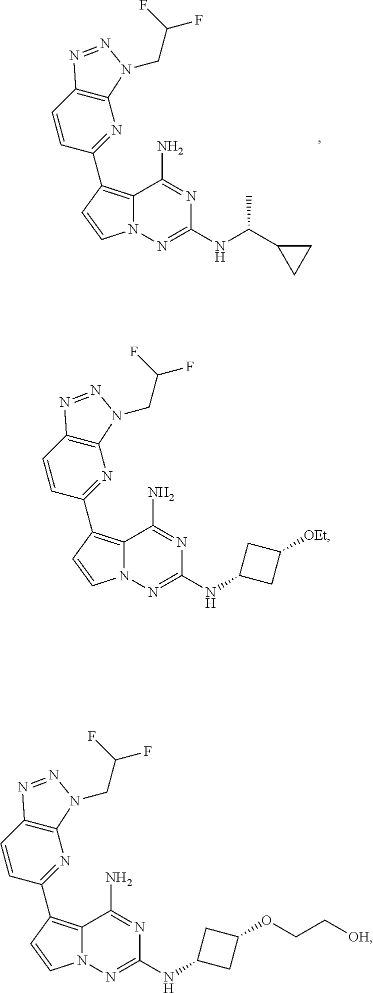 [SMILES: C[C@@H](Nc1nc(N)c2c(-c3ccc4nnn(CC(F)F)c4n3)ccn2n1)C1CC1 ; CCO[C@H]1C[C@@H](Nc2nc(N)c3c(-c4ccc5nnn(CC(F)F)c5n4)ccn3n2)C1 ; Nc1nc(N[C@H]2C[C@@H](OCCO)C2)nn2ccc(-c3ccc4nnn(CC(F)F)c4n3)c12] 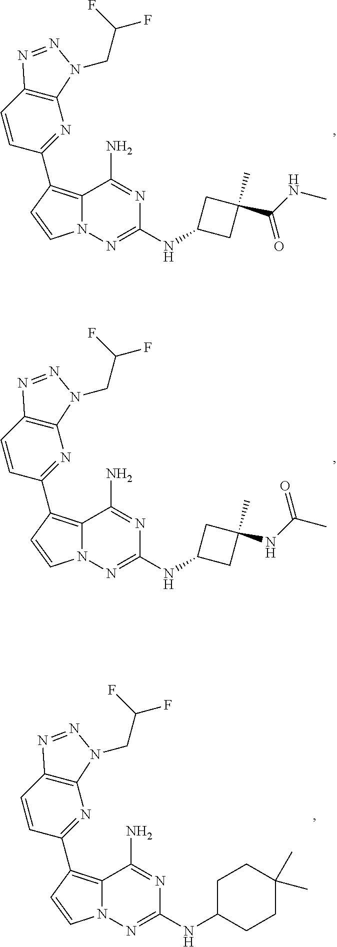 [SMILES: CNC(=O)[C@]1(C)C[C@@H](Nc2nc(N)c3c(-c4ccc5nnn(CC(F)F)c5n4)ccn3n2)C1 ; CC(=O)N[C@]1(C)C[C@@H](Nc2nc(N)c3c(-c4ccc5nnn(CC(F)F)c5n4)ccn3n2)C1 ; CC1(C)CCC(Nc2nc(N)c3c(-c4ccc5nnn(CC(F)F)c5n4)ccn3n2)CC1] 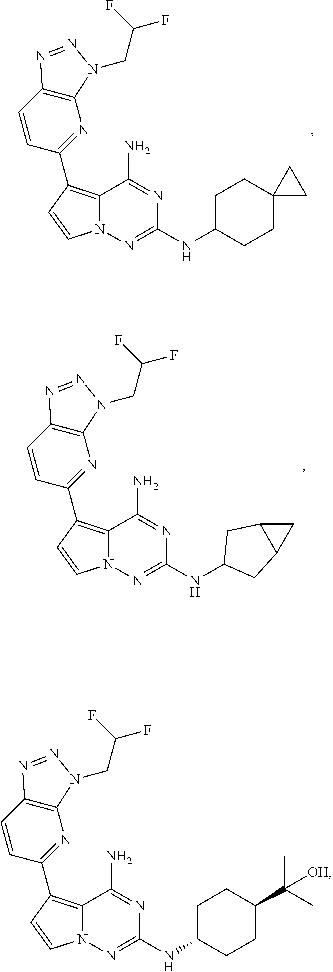 [SMILES: Nc1nc(NC2CCC3(CC2)CC3)nn2ccc(-c3ccc4nnn(CC(F)F)c4n3)c12 ; Nc1nc(NC2CC3CC3C2)nn2ccc(-c3ccc4nnn(CC(F)F)c4n3)c12 ; CC(C)(O)[C@H]1CC[C@H](Nc2nc(N)c3c(-c4ccc5nnn(CC(F)F)c5n4)ccn3n2)CC1] 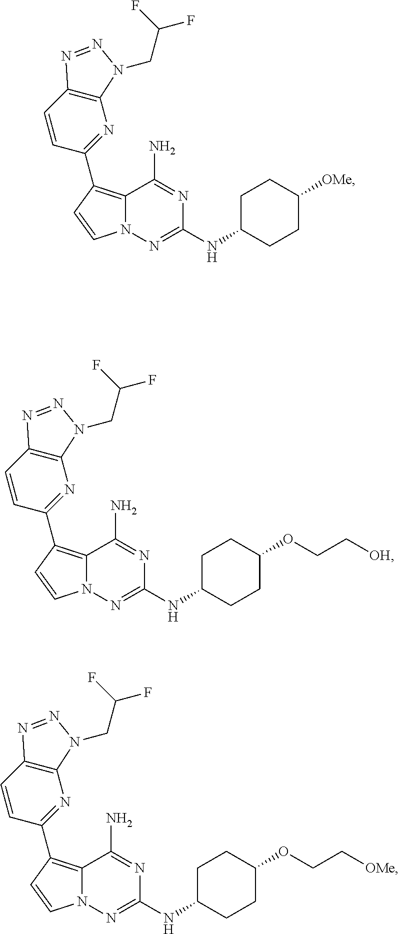 [SMILES: CO[C@H]1CC[C@@H](Nc2nc(N)c3c(-c4ccc5nnn(CC(F)F)c5n4)ccn3n2)CC1 ; Nc1nc(N[C@H]2CC[C@@H](OCCO)CC2)nn2ccc(-c3ccc4nnn(CC(F)F)c4n3)c12 ; COCCO[C@H]1CC[C@@H](Nc2nc(N)c3c(-c4ccc5nnn(CC(F)F)c5n4)ccn3n2)CC1] 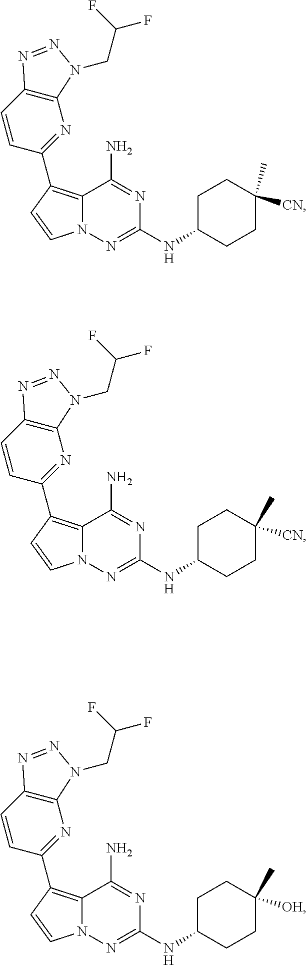 [SMILES: C[C@]1(C#N)CC[C@H](Nc2nc(N)c3c(-c4ccc5nnn(CC(F)F)c5n4)ccn3n2)CC1 ; C[C@]1(C#N)CC[C@@H](Nc2nc(N)c3c(-c4ccc5nnn(CC(F)F)c5n4)ccn3n2)CC1 ; C[C@]1(O)CC[C@@H](Nc2nc(N)c3c(-c4ccc5nnn(CC(F)F)c5n4)ccn3n2)CC1] 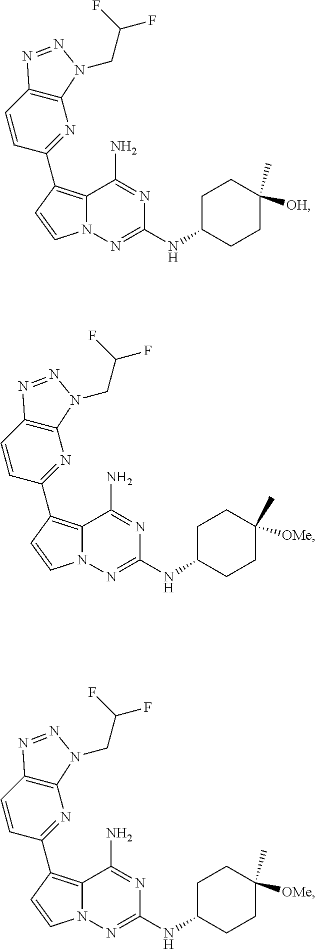 [SMILES: C[C@]1(O)CC[C@H](Nc2nc(N)c3c(-c4ccc5nnn(CC(F)F)c5n4)ccn3n2)CC1 ; CO[C@]1(C)CC[C@H](Nc2nc(N)c3c(-c4ccc5nnn(CC(F)F)c5n4)ccn3n2)CC1 ; CO[C@]1(C)CC[C@@H](Nc2nc(N)c3c(-c4ccc5nnn(CC(F)F)c5n4)ccn3n2)CC1] 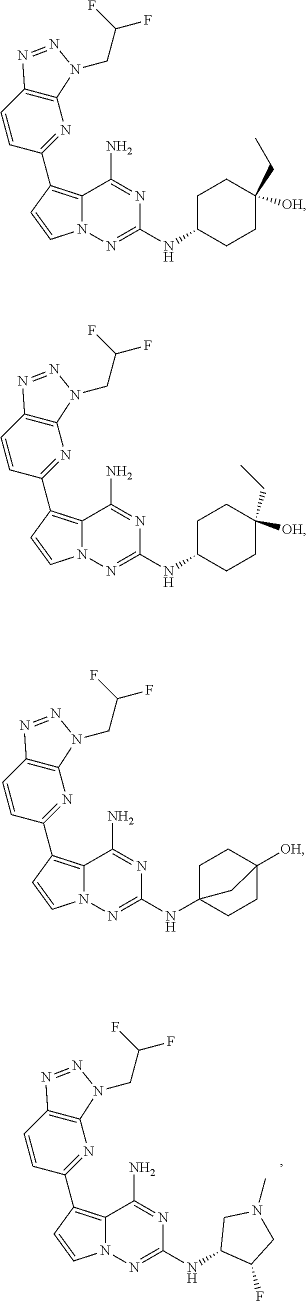 [SMILES: CC[C@]1(O)CC[C@@H](Nc2nc(N)c3c(-c4ccc5nnn(CC(F)F)c5n4)ccn3n2)CC1 ; CC[C@]1(O)CC[C@H](Nc2nc(N)c3c(-c4ccc5nnn(CC(F)F)c5n4)ccn3n2)CC1 ; Nc1nc(NC23CCC(O)(CC2)C3)nn2ccc(-c3ccc4nnn(CC(F)F)c4n3)c12 ; CN1C[C@H](F)[C@H](Nc2nc(N)c3c(-c4ccc5nnn(CC(F)F)c5n4)ccn3n2)C1] 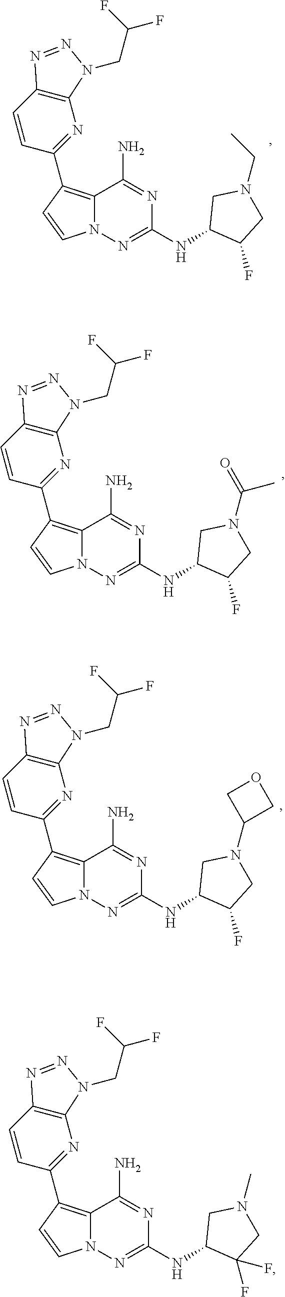 [SMILES: CCN1C[C@H](F)[C@H](Nc2nc(N)c3c(-c4ccc5nnn(CC(F)F)c5n4)ccn3n2)C1 ; CC(=O)N1C[C@H](F)[C@H](Nc2nc(N)c3c(-c4ccc5nnn(CC(F)F)c5n4)ccn3n2)C1 ; Nc1nc(N[C@@H]2CN(C3COC3)C[C@@H]2F)nn2ccc(-c3ccc4nnn(CC(F)F)c4n3)c12 ; CN1C[C@@H](Nc2nc(N)c3c(-c4ccc5nnn(CC(F)F)c5n4)ccn3n2)C(F)(F)C1] 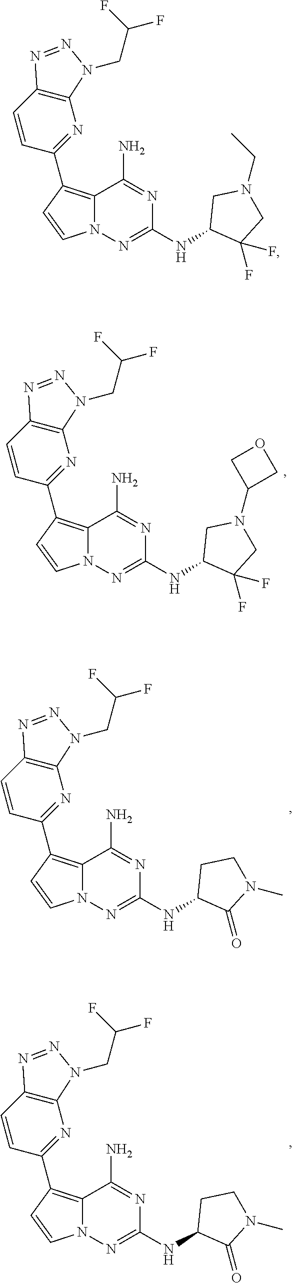 [SMILES: CCN1C[C@@H](Nc2nc(N)c3c(-c4ccc5nnn(CC(F)F)c5n4)ccn3n2)C(F)(F)C1 ; Nc1nc(N[C@@H]2CN(C3COC3)CC2(F)F)nn2ccc(-c3ccc4nnn(CC(F)F)c4n3)c12 ; CN1CC[C@@H](Nc2nc(N)c3c(-c4ccc5nnn(CC(F)F)c5n4)ccn3n2)C1=O ; CN1CC[C@H](Nc2nc(N)c3c(-c4ccc5nnn(CC(F)F)c5n4)ccn3n2)C1=O] 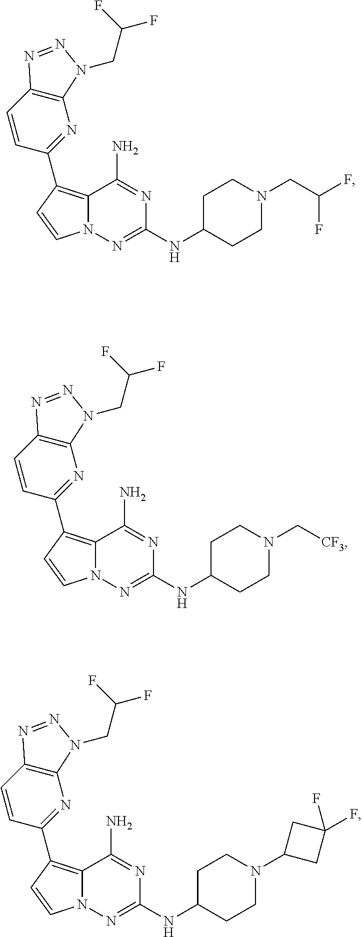 [SMILES: Nc1nc(NC2CCN(CC(F)F)CC2)nn2ccc(-c3ccc4nnn(CC(F)F)c4n3)c12 ; Nc1nc(NC2CCN(CC(F)(F)F)CC2)nn2ccc(-c3ccc4nnn(CC(F)F)c4n3)c12 ; Nc1nc(NC2CCN(C3CC(F)(F)C3)CC2)nn2ccc(-c3ccc4nnn(CC(F)F)c4n3)c12] 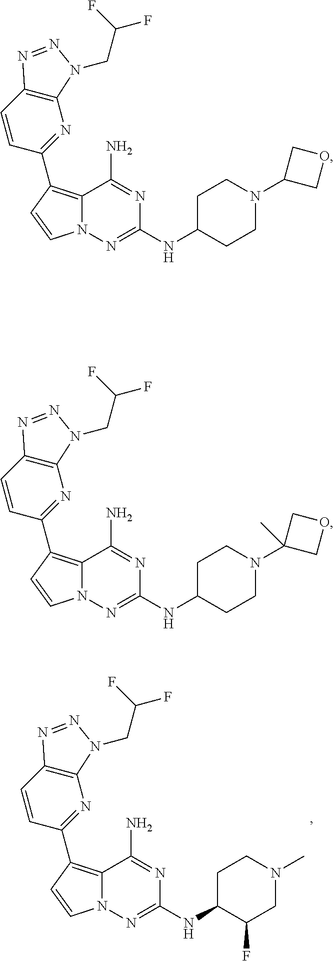 [SMILES: Nc1nc(NC2CCN(C3COC3)CC2)nn2ccc(-c3ccc4nnn(CC(F)F)c4n3)c12 ; CC1(N2CCC(Nc3nc(N)c4c(-c5ccc6nnn(CC(F)F)c6n5)ccn4n3)CC2)COC1 ; CN1CC[C@H](Nc2nc(N)c3c(-c4ccc5nnn(CC(F)F)c5n4)ccn3n2)[C@H](F)C1] 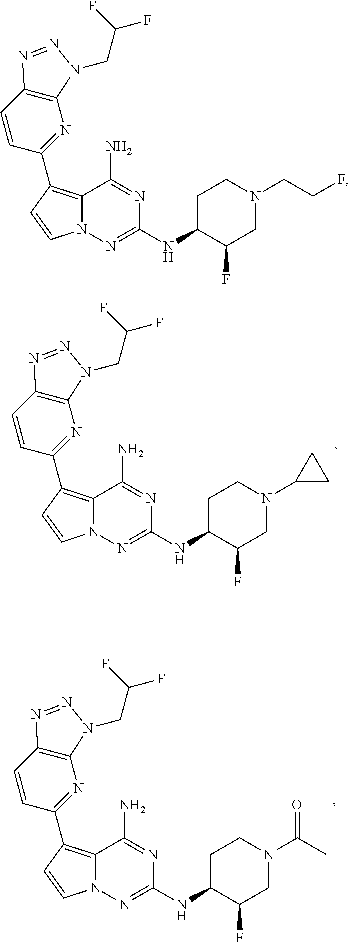 [SMILES: Nc1nc(N[C@H]2CCN(CCF)C[C@H]2F)nn2ccc(-c3ccc4nnn(CC(F)F)c4n3)c12 ; Nc1nc(N[C@H]2CCN(C3CC3)C[C@H]2F)nn2ccc(-c3ccc4nnn(CC(F)F)c4n3)c12 ; CC(=O)N1CC[C@H](Nc2nc(N)c3c(-c4ccc5nnn(CC(F)F)c5n4)ccn3n2)[C@H](F)C1] 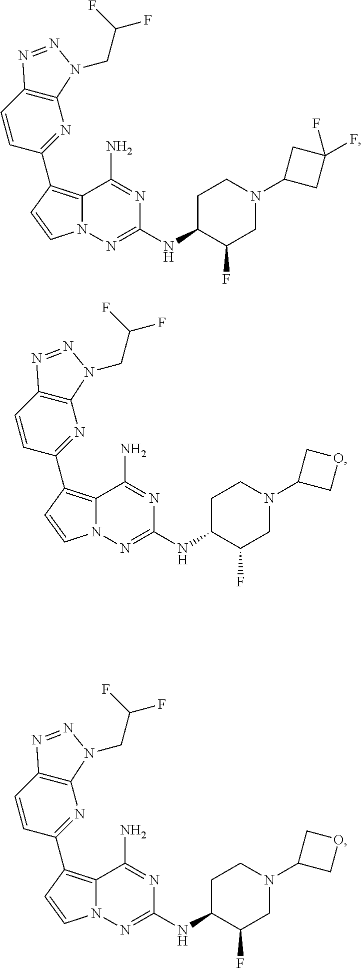 [SMILES: Nc1nc(N[C@H]2CCN(C3CC(F)(F)C3)C[C@H]2F)nn2ccc(-c3ccc4nnn(CC(F)F)c4n3)c12 ; Nc1nc(N[C@@H]2CCN(C3COC3)C[C@@H]2F)nn2ccc(-c3ccc4nnn(CC(F)F)c4n3)c12 ; Nc1nc(N[C@H]2CCN(C3COC3)C[C@H]2F)nn2ccc(-c3ccc4nnn(CC(F)F)c4n3)c12] 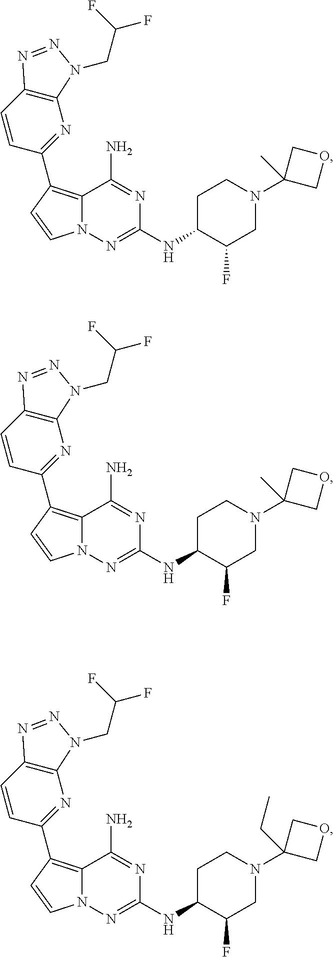 [SMILES: CC1(N2CC[C@@H](Nc3nc(N)c4c(-c5ccc6nnn(CC(F)F)c6n5)ccn4n3)[C@@H](F)C2)COC1 ; CC1(N2CC[C@H](Nc3nc(N)c4c(-c5ccc6nnn(CC(F)F)c6n5)ccn4n3)[C@H](F)C2)COC1 ; CCC1(N2CC[C@H](Nc3nc(N)c4c(-c5ccc6nnn(CC(F)F)c6n5)ccn4n3)[C@H](F)C2)COC1] 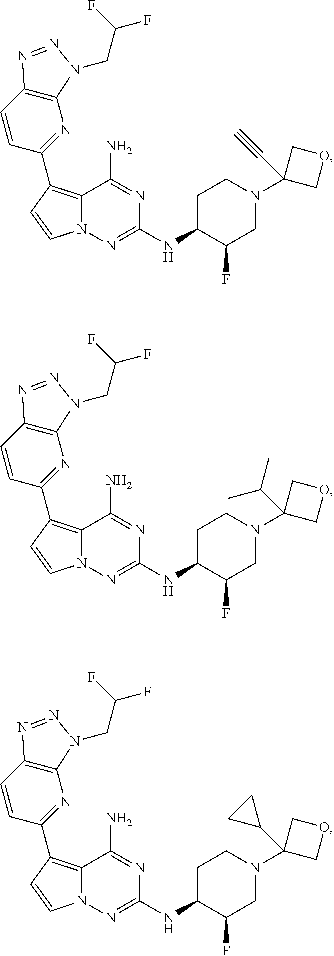 [SMILES: C#CC1(N2CC[C@H](Nc3nc(N)c4c(-c5ccc6nnn(CC(F)F)c6n5)ccn4n3)[C@H](F)C2)COC1 ; CC(C)C1(N2CC[C@H](Nc3nc(N)c4c(-c5ccc6nnn(CC(F)F)c6n5)ccn4n3)[C@H](F)C2)COC1 ; Nc1nc(N[C@H]2CCN(C3(C4CC4)COC3)C[C@H]2F)nn2ccc(-c3ccc4nnn(CC(F)F)c4n3)c12] 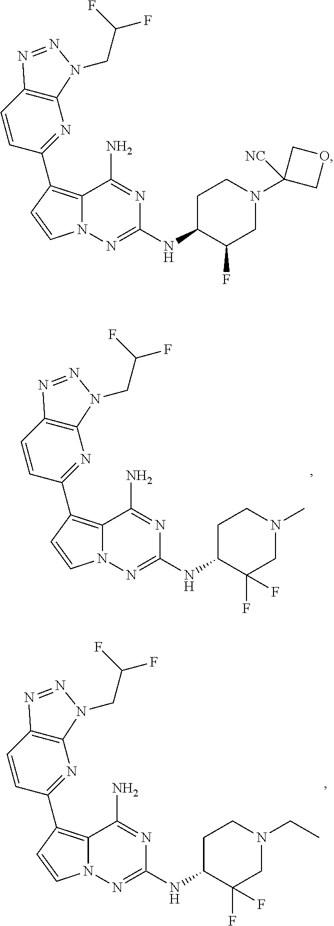 [SMILES: N#CC1(N2CC[C@H](Nc3nc(N)c4c(-c5ccc6nnn(CC(F)F)c6n5)ccn4n3)[C@H](F)C2)COC1 ; CN1CC[C@@H](Nc2nc(N)c3c(-c4ccc5nnn(CC(F)F)c5n4)ccn3n2)C(F)(F)C1 ; CCN1CC[C@@H](Nc2nc(N)c3c(-c4ccc5nnn(CC(F)F)c5n4)ccn3n2)C(F)(F)C1] 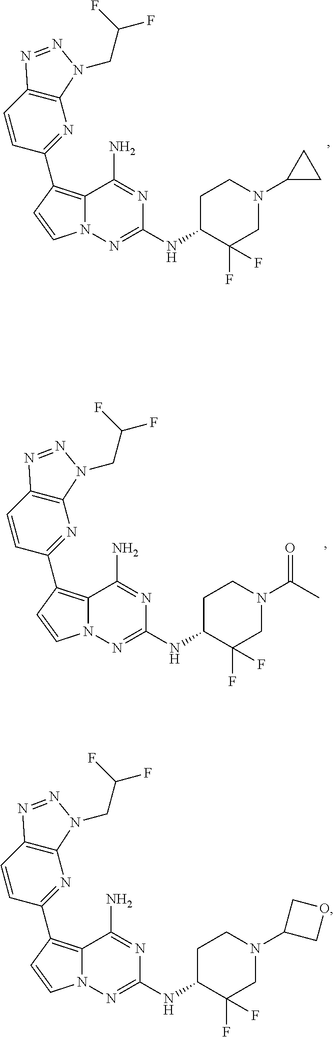 [SMILES: Nc1nc(N[C@@H]2CCN(C3CC3)CC2(F)F)nn2ccc(-c3ccc4nnn(CC(F)F)c4n3)c12 ; CC(=O)N1CC[C@@H](Nc2nc(N)c3c(-c4ccc5nnn(CC(F)F)c5n4)ccn3n2)C(F)(F)C1 ; Nc1nc(N[C@@H]2CCN(C3COC3)CC2(F)F)nn2ccc(-c3ccc4nnn(CC(F)F)c4n3)c12] 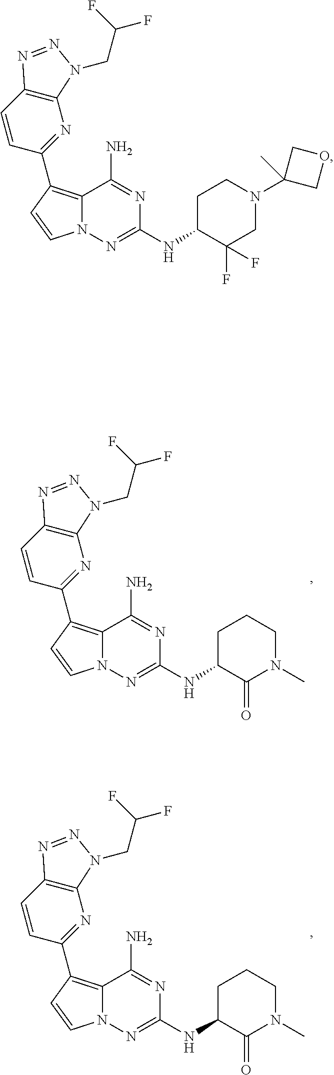 [SMILES: CC1(N2CC[C@@H](Nc3nc(N)c4c(-c5ccc6nnn(CC(F)F)c6n5)ccn4n3)C(F)(F)C2)COC1 ; CN1CCC[C@@H](Nc2nc(N)c3c(-c4ccc5nnn(CC(F)F)c5n4)ccn3n2)C1=O ; CN1CCC[C@H](Nc2nc(N)c3c(-c4ccc5nnn(CC(F)F)c5n4)ccn3n2)C1=O] 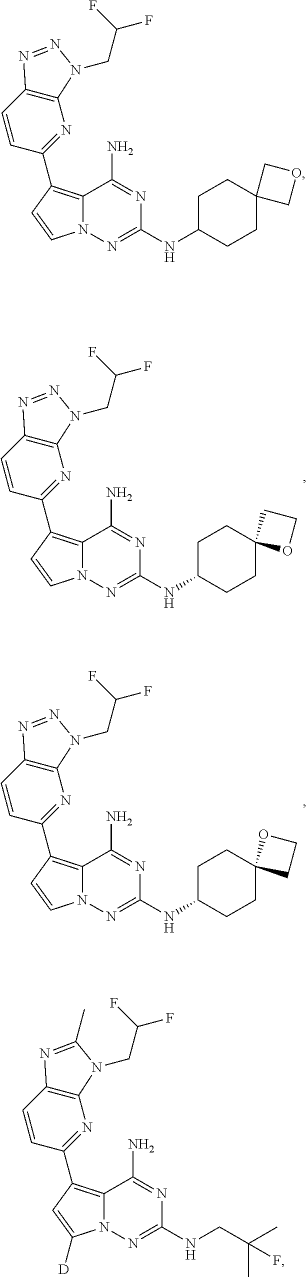 [SMILES: Nc1nc(NC2CCC3(CC2)COC3)nn2ccc(-c3ccc4nnn(CC(F)F)c4n3)c12 ; Nc1nc(N[C@H]2CC[C@@]3(CCO3)CC2)nn2ccc(-c3ccc4nnn(CC(F)F)c4n3)c12 ; Nc1nc(N[C@H]2CC[C@]3(CCO3)CC2)nn2ccc(-c3ccc4nnn(CC(F)F)c4n3)c12 ; [2H]c1cc(-c2ccc3nc(C)n(CC(F)F)c3n2)c2c(N)nc(NCC(C)(C)F)nn12] 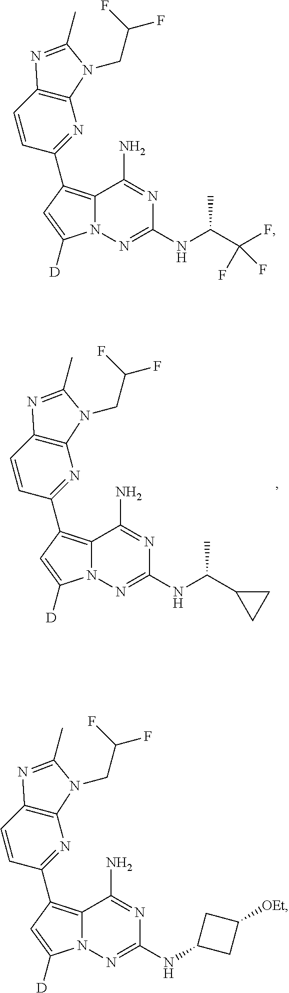 [SMILES: [2H]c1cc(-c2ccc3nc(C)n(CC(F)F)c3n2)c2c(N)nc(N[C@H](C)C(F)(F)F)nn12 ; [2H]c1cc(-c2ccc3nc(C)n(CC(F)F)c3n2)c2c(N)nc(N[C@H](C)C3CC3)nn12 ; [2H]c1cc(-c2ccc3nc(C)n(CC(F)F)c3n2)c2c(N)nc(N[C@H]3C[C@@H](OCC)C3)nn12] 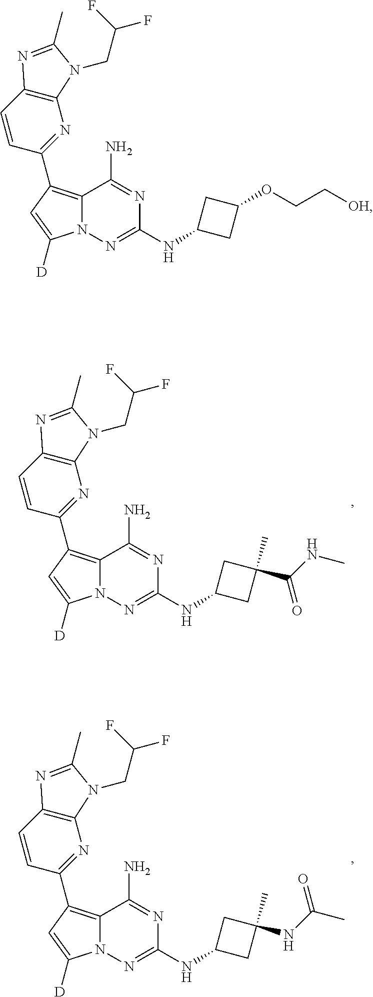 [SMILES: [2H]c1cc(-c2ccc3nc(C)n(CC(F)F)c3n2)c2c(N)nc(N[C@H]3C[C@@H](OCCO)C3)nn12 ; [2H]c1cc(-c2ccc3nc(C)n(CC(F)F)c3n2)c2c(N)nc(N[C@H]3C[C@](C)(C(=O)NC)C3)nn12 ; [2H]c1cc(-c2ccc3nc(C)n(CC(F)F)c3n2)c2c(N)nc(N[C@H]3C[C@](C)(NC(C)=O)C3)nn12] 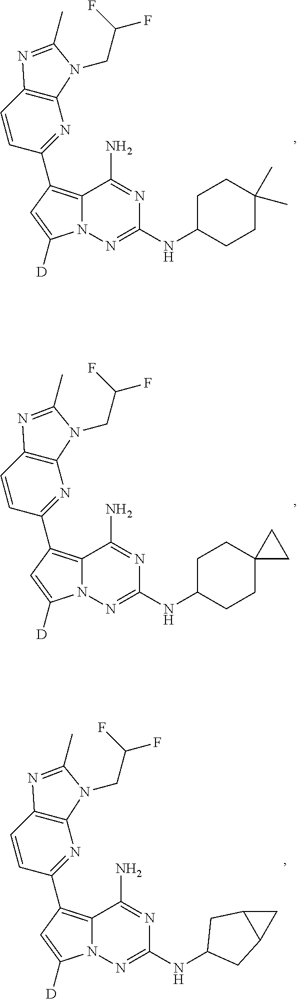 [SMILES: [2H]c1cc(-c2ccc3nc(C)n(CC(F)F)c3n2)c2c(N)nc(NC3CCC(C)(C)CC3)nn12 ; [2H]c1cc(-c2ccc3nc(C)n(CC(F)F)c3n2)c2c(N)nc(NC3CCC4(CC3)CC4)nn12 ; [2H]c1cc(-c2ccc3nc(C)n(CC(F)F)c3n2)c2c(N)nc(NC3CC4CC4C3)nn12] 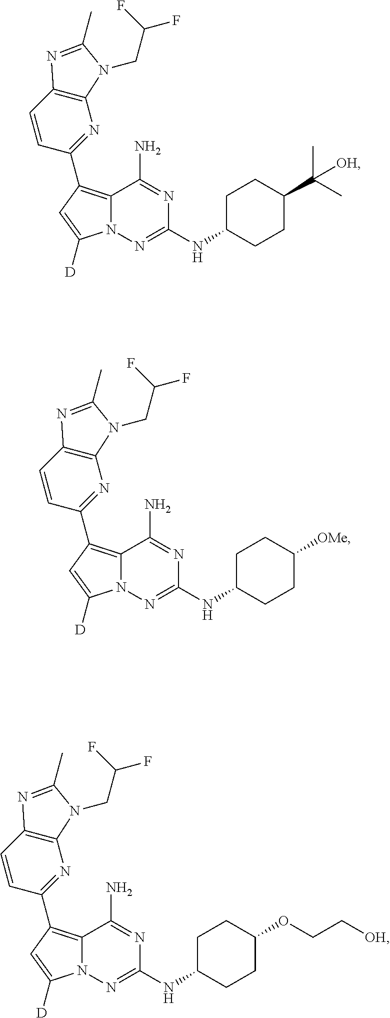 [SMILES: [2H]c1cc(-c2ccc3nc(C)n(CC(F)F)c3n2)c2c(N)nc(N[C@H]3CC[C@H](C(C)(C)O)CC3)nn12 ; [2H]c1cc(-c2ccc3nc(C)n(CC(F)F)c3n2)c2c(N)nc(N[C@H]3CC[C@@H](OC)CC3)nn12 ; [2H]c1cc(-c2ccc3nc(C)n(CC(F)F)c3n2)c2c(N)nc(N[C@H]3CC[C@@H](OCCO)CC3)nn12] 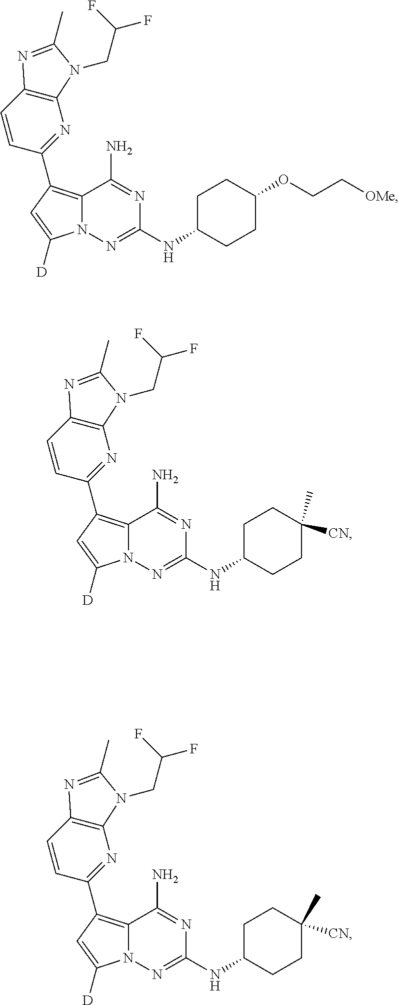 [SMILES: [2H]c1cc(-c2ccc3nc(C)n(CC(F)F)c3n2)c2c(N)nc(N[C@H]3CC[C@@H](OCCOC)CC3)nn12 ; [2H]c1cc(-c2ccc3nc(C)n(CC(F)F)c3n2)c2c(N)nc(N[C@H]3CC[C@](C)(C#N)CC3)nn12 ; [2H]c1cc(-c2ccc3nc(C)n(CC(F)F)c3n2)c2c(N)nc(N[C@H]3CC[C@@](C)(C#N)CC3)nn12] 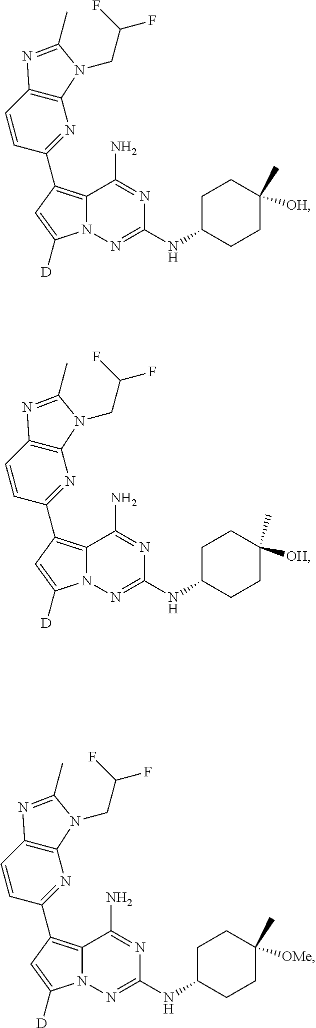 [SMILES: [2H]c1cc(-c2ccc3nc(C)n(CC(F)F)c3n2)c2c(N)nc(N[C@H]3CC[C@@](C)(O)CC3)nn12 ; [2H]c1cc(-c2ccc3nc(C)n(CC(F)F)c3n2)c2c(N)nc(N[C@H]3CC[C@](C)(O)CC3)nn12 ; [2H]c1cc(-c2ccc3nc(C)n(CC(F)F)c3n2)c2c(N)nc(N[C@H]3CC[C@@](C)(OC)CC3)nn12] 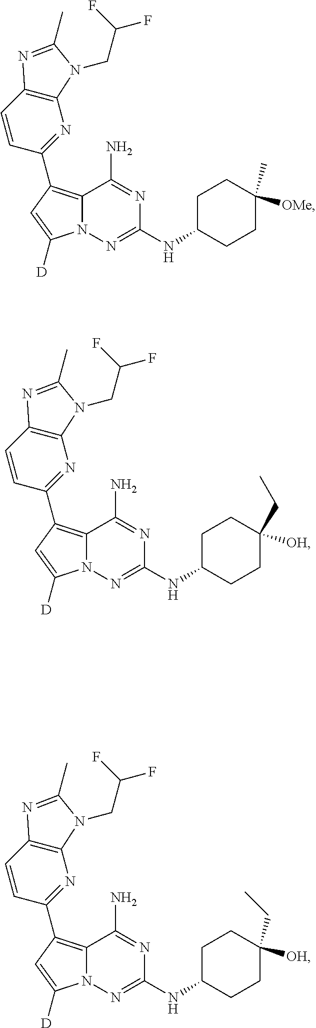 [SMILES: [2H]c1cc(-c2ccc3nc(C)n(CC(F)F)c3n2)c2c(N)nc(N[C@H]3CC[C@](C)(OC)CC3)nn12 ; [2H]c1cc(-c2ccc3nc(C)n(CC(F)F)c3n2)c2c(N)nc(N[C@H]3CC[C@](O)(CC)CC3)nn12 ; [2H]c1cc(-c2ccc3nc(C)n(CC(F)F)c3n2)c2c(N)nc(N[C@H]3CC[C@@](O)(CC)CC3)nn12] 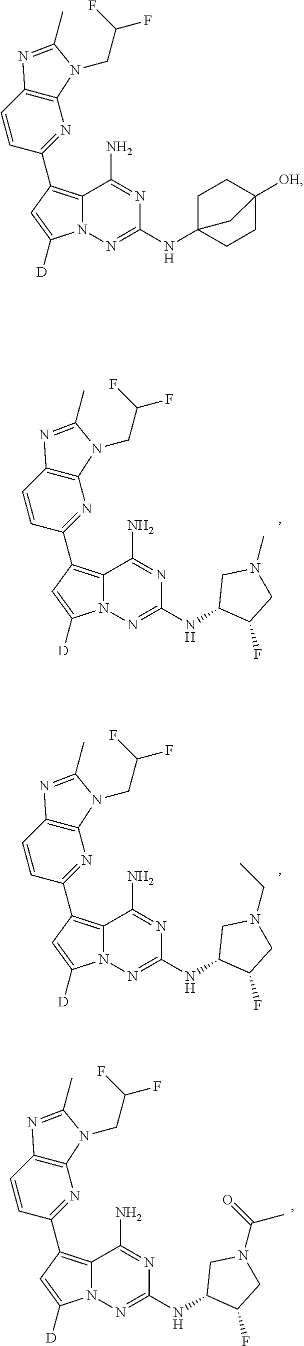 [SMILES: [2H]c1cc(-c2ccc3nc(C)n(CC(F)F)c3n2)c2c(N)nc(NC34CCC(O)(CC3)C4)nn12 ; [2H]c1cc(-c2ccc3nc(C)n(CC(F)F)c3n2)c2c(N)nc(N[C@@H]3CN(C)C[C@@H]3F)nn12 ; [2H]c1cc(-c2ccc3nc(C)n(CC(F)F)c3n2)c2c(N)nc(N[C@@H]3CN(CC)C[C@@H]3F)nn12 ; [2H]c1cc(-c2ccc3nc(C)n(CC(F)F)c3n2)c2c(N)nc(N[C@@H]3CN(C(C)=O)C[C@@H]3F)nn12] 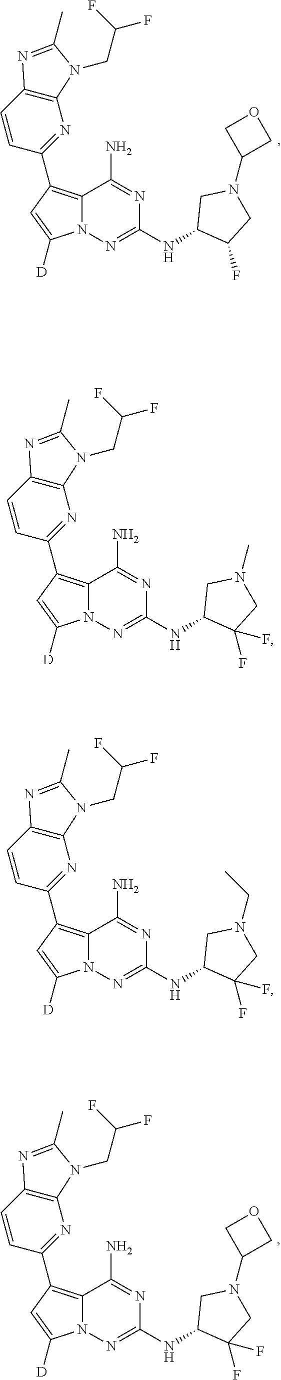 [SMILES: [2H]c1cc(-c2ccc3nc(C)n(CC(F)F)c3n2)c2c(N)nc(N[C@@H]3CN(C4COC4)C[C@@H]3F)nn12 ; [2H]c1cc(-c2ccc3nc(C)n(CC(F)F)c3n2)c2c(N)nc(N[C@@H]3CN(C)CC3(F)F)nn12 ; [2H]c1cc(-c2ccc3nc(C)n(CC(F)F)c3n2)c2c(N)nc(N[C@@H]3CN(CC)CC3(F)F)nn12 ; [2H]c1cc(-c2ccc3nc(C)n(CC(F)F)c3n2)c2c(N)nc(N[C@@H]3CN(C4COC4)CC3(F)F)nn12] 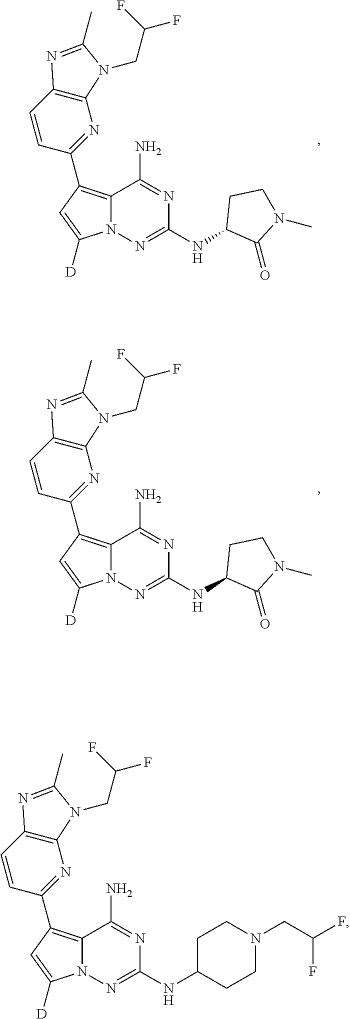 [SMILES: [2H]c1cc(-c2ccc3nc(C)n(CC(F)F)c3n2)c2c(N)nc(N[C@@H]3CCN(C)C3=O)nn12 ; [2H]c1cc(-c2ccc3nc(C)n(CC(F)F)c3n2)c2c(N)nc(N[C@H]3CCN(C)C3=O)nn12 ; [2H]c1cc(-c2ccc3nc(C)n(CC(F)F)c3n2)c2c(N)nc(NC3CCN(CC(F)F)CC3)nn12] 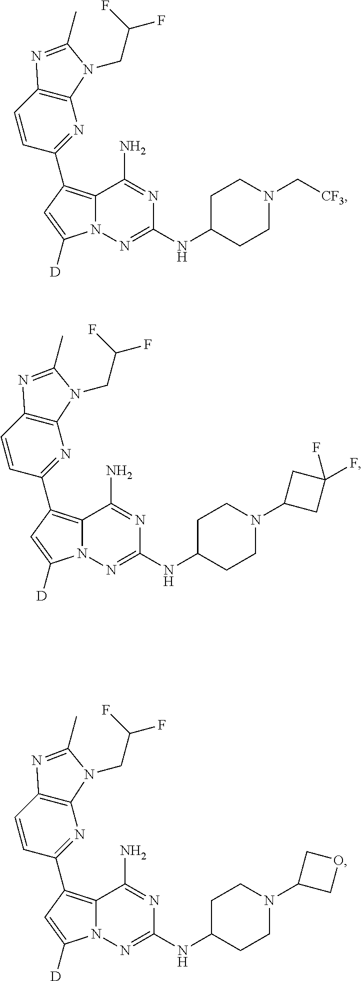 [SMILES: [2H]c1cc(-c2ccc3nc(C)n(CC(F)F)c3n2)c2c(N)nc(NC3CCN(CC(F)(F)F)CC3)nn12 ; [2H]c1cc(-c2ccc3nc(C)n(CC(F)F)c3n2)c2c(N)nc(NC3CCN(C4CC(F)(F)C4)CC3)nn12 ; [2H]c1cc(-c2ccc3nc(C)n(CC(F)F)c3n2)c2c(N)nc(NC3CCN(C4COC4)CC3)nn12] 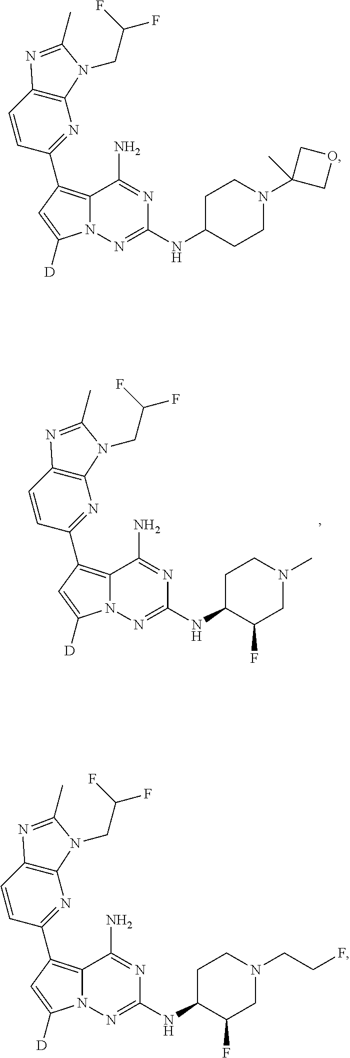 [SMILES: [2H]c1cc(-c2ccc3nc(C)n(CC(F)F)c3n2)c2c(N)nc(NC3CCN(C4(C)COC4)CC3)nn12 ; [2H]c1cc(-c2ccc3nc(C)n(CC(F)F)c3n2)c2c(N)nc(N[C@H]3CCN(C)C[C@H]3F)nn12 ; [2H]c1cc(-c2ccc3nc(C)n(CC(F)F)c3n2)c2c(N)nc(N[C@H]3CCN(CCF)C[C@H]3F)nn12] 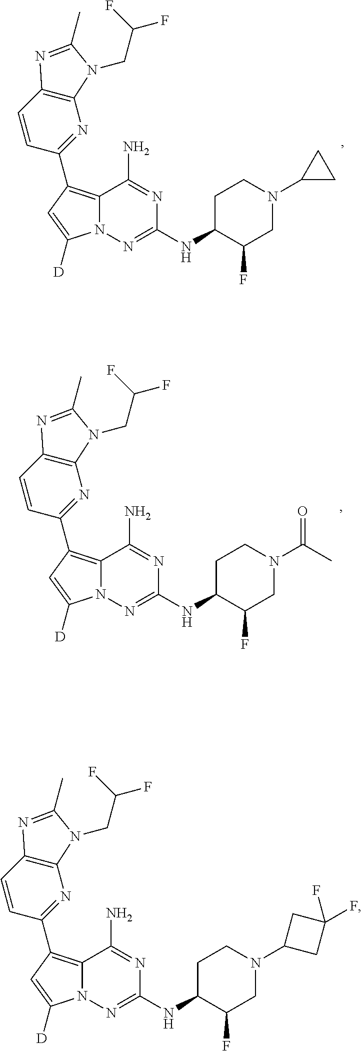 [SMILES: [2H]c1cc(-c2ccc3nc(C)n(CC(F)F)c3n2)c2c(N)nc(N[C@H]3CCN(C4CC4)C[C@H]3F)nn12 ; [2H]c1cc(-c2ccc3nc(C)n(CC(F)F)c3n2)c2c(N)nc(N[C@H]3CCN(C(C)=O)C[C@H]3F)nn12 ; [2H]c1cc(-c2ccc3nc(C)n(CC(F)F)c3n2)c2c(N)nc(N[C@H]3CCN(C4CC(F)(F)C4)C[C@H]3F)nn12] 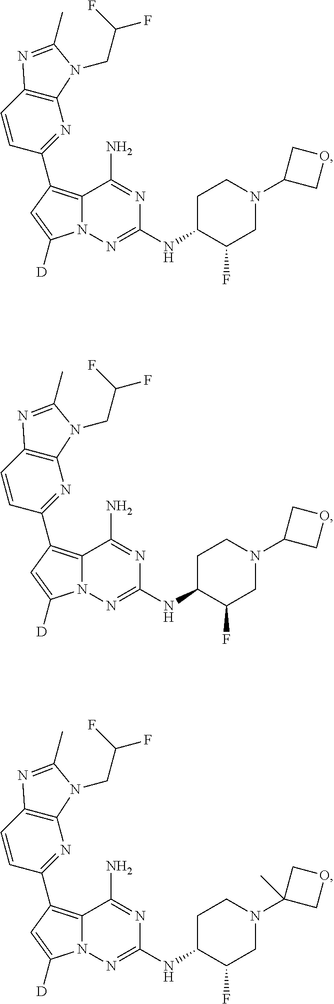 [SMILES: [2H]c1cc(-c2ccc3nc(C)n(CC(F)F)c3n2)c2c(N)nc(N[C@@H]3CCN(C4COC4)C[C@@H]3F)nn12 ; [2H]c1cc(-c2ccc3nc(C)n(CC(F)F)c3n2)c2c(N)nc(N[C@H]3CCN(C4COC4)C[C@H]3F)nn12 ; [2H]c1cc(-c2ccc3nc(C)n(CC(F)F)c3n2)c2c(N)nc(N[C@@H]3CCN(C4(C)COC4)C[C@@H]3F)nn12] 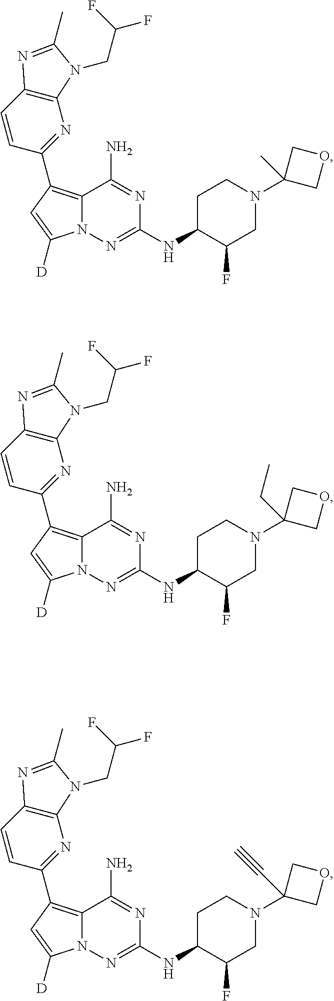 [SMILES: [2H]c1cc(-c2ccc3nc(C)n(CC(F)F)c3n2)c2c(N)nc(N[C@H]3CCN(C4(C)COC4)C[C@H]3F)nn12 ; [2H]c1cc(-c2ccc3nc(C)n(CC(F)F)c3n2)c2c(N)nc(N[C@H]3CCN(C4(CC)COC4)C[C@H]3F)nn12 ; [2H]c1cc(-c2ccc3nc(C)n(CC(F)F)c3n2)c2c(N)nc(N[C@H]3CCN(C4(C#C)COC4)C[C@H]3F)nn12] 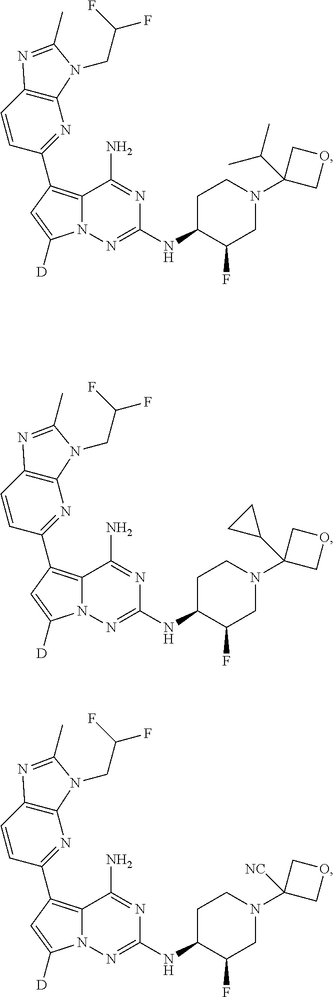 [SMILES: [2H]c1cc(-c2ccc3nc(C)n(CC(F)F)c3n2)c2c(N)nc(N[C@H]3CCN(C4(C(C)C)COC4)C[C@H]3F)nn12 ; [2H]c1cc(-c2ccc3nc(C)n(CC(F)F)c3n2)c2c(N)nc(N[C@H]3CCN(C4(C5CC5)COC4)C[C@H]3F)nn12 ; [2H]c1cc(-c2ccc3nc(C)n(CC(F)F)c3n2)c2c(N)nc(N[C@H]3CCN(C4(C#N)COC4)C[C@H]3F)nn12] 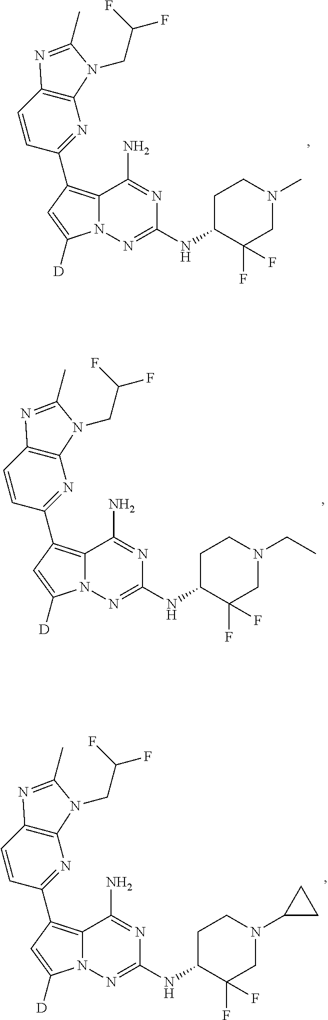 [SMILES: [2H]c1cc(-c2ccc3nc(C)n(CC(F)F)c3n2)c2c(N)nc(N[C@@H]3CCN(C)CC3(F)F)nn12 ; [2H]c1cc(-c2ccc3nc(C)n(CC(F)F)c3n2)c2c(N)nc(N[C@@H]3CCN(CC)CC3(F)F)nn12 ; [2H]c1cc(-c2ccc3nc(C)n(CC(F)F)c3n2)c2c(N)nc(N[C@@H]3CCN(C4CC4)CC3(F)F)nn12] 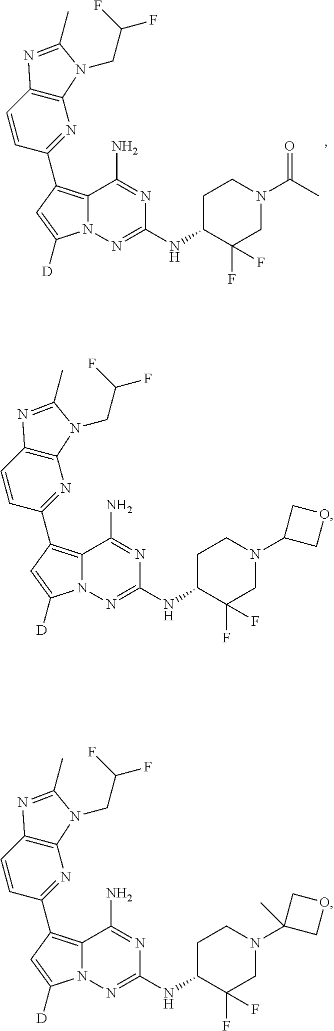 [SMILES: [2H]c1cc(-c2ccc3nc(C)n(CC(F)F)c3n2)c2c(N)nc(N[C@@H]3CCN(C(C)=O)CC3(F)F)nn12 ; [2H]c1cc(-c2ccc3nc(C)n(CC(F)F)c3n2)c2c(N)nc(N[C@@H]3CCN(C4COC4)CC3(F)F)nn12 ; [2H]c1cc(-c2ccc3nc(C)n(CC(F)F)c3n2)c2c(N)nc(N[C@@H]3CCN(C4(C)COC4)CC3(F)F)nn12] 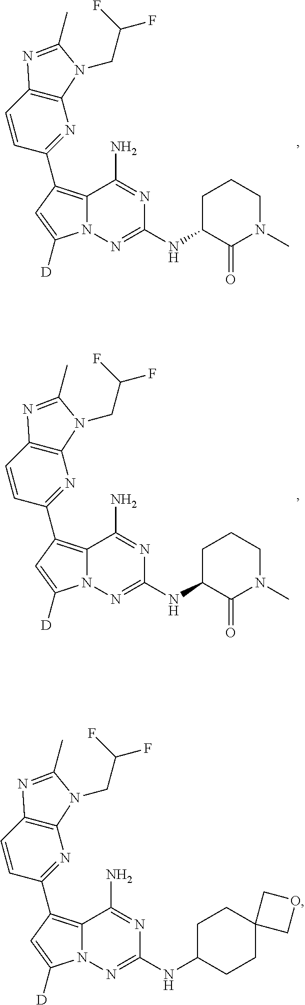 [SMILES: [2H]c1cc(-c2ccc3nc(C)n(CC(F)F)c3n2)c2c(N)nc(N[C@@H]3CCCN(C)C3=O)nn12 ; [2H]c1cc(-c2ccc3nc(C)n(CC(F)F)c3n2)c2c(N)nc(N[C@H]3CCCN(C)C3=O)nn12 ; [2H]c1cc(-c2ccc3nc(C)n(CC(F)F)c3n2)c2c(N)nc(NC3CCC4(CC3)COC4)nn12] 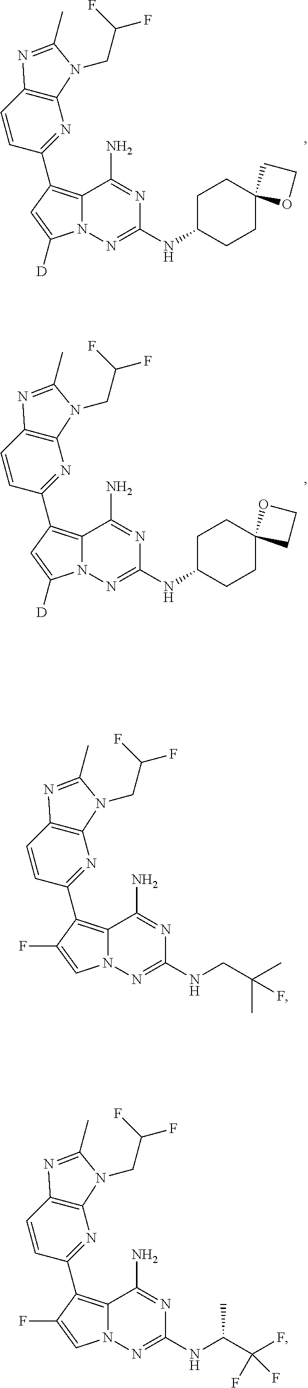 [SMILES: [2H]c1cc(-c2ccc3nc(C)n(CC(F)F)c3n2)c2c(N)nc(N[C@H]3CC[C@@]4(CCO4)CC3)nn12 ; [2H]c1cc(-c2ccc3nc(C)n(CC(F)F)c3n2)c2c(N)nc(N[C@H]3CC[C@]4(CCO4)CC3)nn12 ; Cc1nc2ccc(-c3c(F)cn4nc(NCC(C)(C)F)nc(N)c34)nc2n1CC(F)F ; Cc1nc2ccc(-c3c(F)cn4nc(N[C@H](C)C(F)(F)F)nc(N)c34)nc2n1CC(F)F] 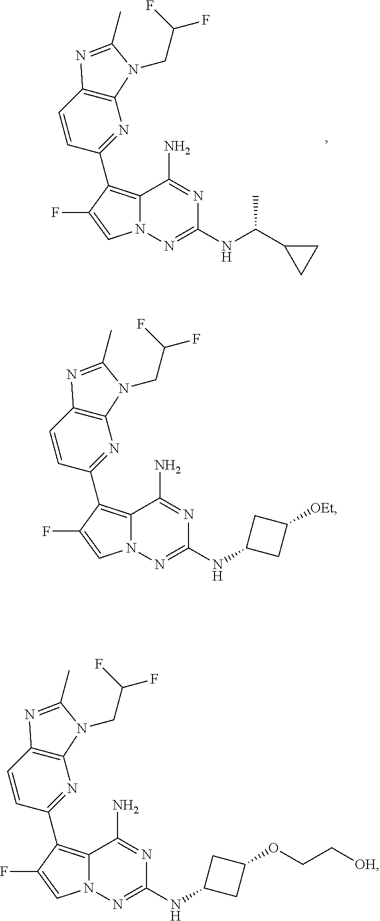 [SMILES: Cc1nc2ccc(-c3c(F)cn4nc(N[C@H](C)C5CC5)nc(N)c34)nc2n1CC(F)F ; CCO[C@H]1C[C@@H](Nc2nc(N)c3c(-c4ccc5nc(C)n(CC(F)F)c5n4)c(F)cn3n2)C1 ; Cc1nc2ccc(-c3c(F)cn4nc(N[C@H]5C[C@@H](OCCO)C5)nc(N)c34)nc2n1CC(F)F] 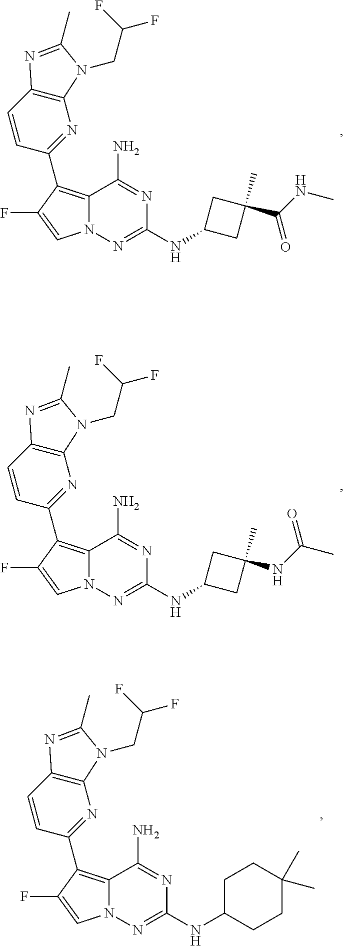 [SMILES: CNC(=O)[C@]1(C)C[C@@H](Nc2nc(N)c3c(-c4ccc5nc(C)n(CC(F)F)c5n4)c(F)cn3n2)C1 ; CC(=O)N[C@]1(C)C[C@@H](Nc2nc(N)c3c(-c4ccc5nc(C)n(CC(F)F)c5n4)c(F)cn3n2)C1 ; Cc1nc2ccc(-c3c(F)cn4nc(NC5CCC(C)(C)CC5)nc(N)c34)nc2n1CC(F)F] 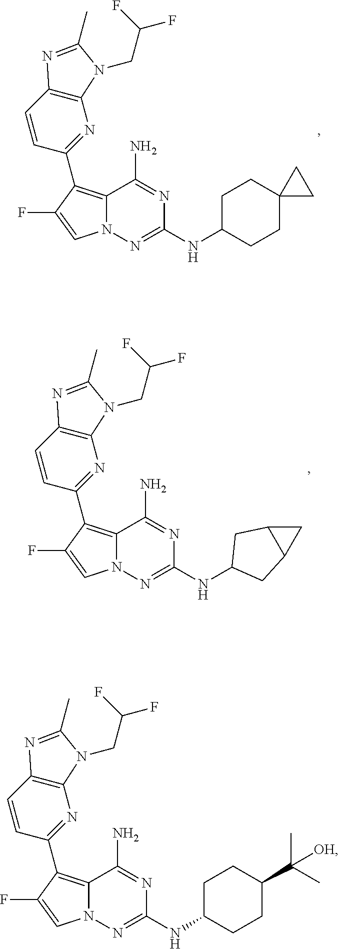 [SMILES: Cc1nc2ccc(-c3c(F)cn4nc(NC5CCC6(CC5)CC6)nc(N)c34)nc2n1CC(F)F ; Cc1nc2ccc(-c3c(F)cn4nc(NC5CC6CC6C5)nc(N)c34)nc2n1CC(F)F ; Cc1nc2ccc(-c3c(F)cn4nc(N[C@H]5CC[C@H](C(C)(C)O)CC5)nc(N)c34)nc2n1CC(F)F] 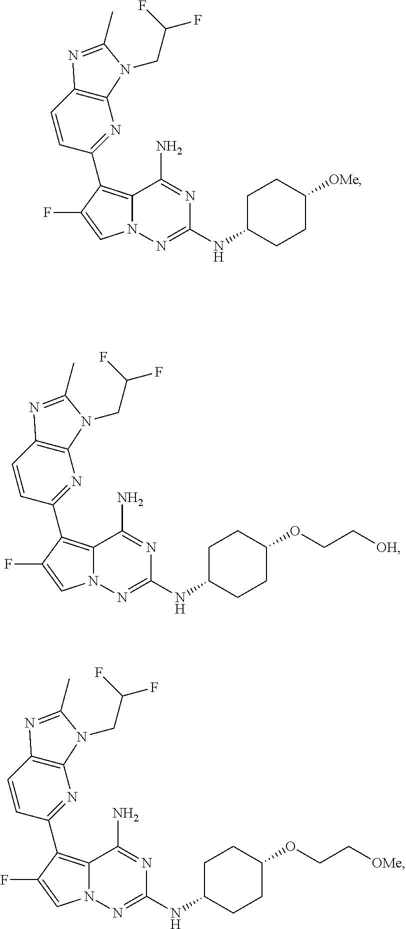 [SMILES: CO[C@H]1CC[C@@H](Nc2nc(N)c3c(-c4ccc5nc(C)n(CC(F)F)c5n4)c(F)cn3n2)CC1 ; Cc1nc2ccc(-c3c(F)cn4nc(N[C@H]5CC[C@@H](OCCO)CC5)nc(N)c34)nc2n1CC(F)F ; COCCO[C@H]1CC[C@@H](Nc2nc(N)c3c(-c4ccc5nc(C)n(CC(F)F)c5n4)c(F)cn3n2)CC1] 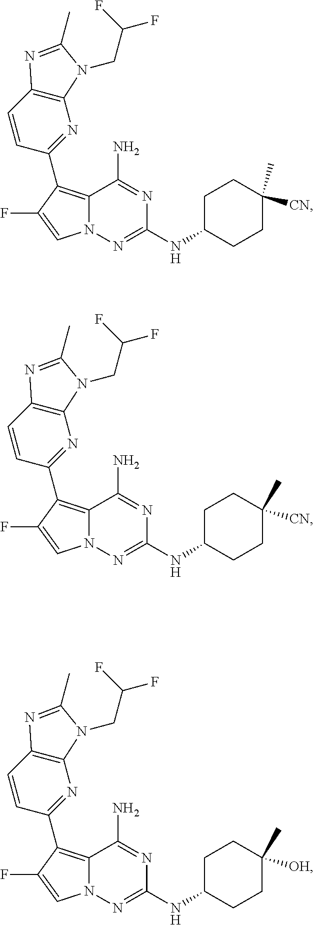 [SMILES: Cc1nc2ccc(-c3c(F)cn4nc(N[C@H]5CC[C@](C)(C#N)CC5)nc(N)c34)nc2n1CC(F)F ; Cc1nc2ccc(-c3c(F)cn4nc(N[C@H]5CC[C@@](C)(C#N)CC5)nc(N)c34)nc2n1CC(F)F ; Cc1nc2ccc(-c3c(F)cn4nc(N[C@H]5CC[C@@](C)(O)CC5)nc(N)c34)nc2n1CC(F)F] 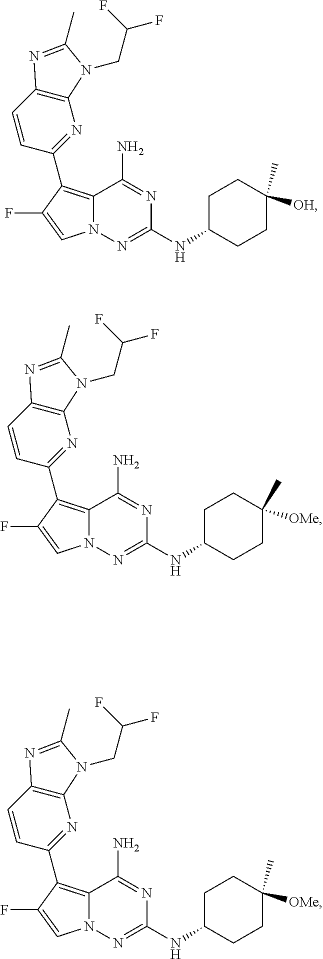 [SMILES: Cc1nc2ccc(-c3c(F)cn4nc(N[C@H]5CC[C@](C)(O)CC5)nc(N)c34)nc2n1CC(F)F ; CO[C@]1(C)CC[C@H](Nc2nc(N)c3c(-c4ccc5nc(C)n(CC(F)F)c5n4)c(F)cn3n2)CC1 ; CO[C@]1(C)CC[C@@H](Nc2nc(N)c3c(-c4ccc5nc(C)n(CC(F)F)c5n4)c(F)cn3n2)CC1] 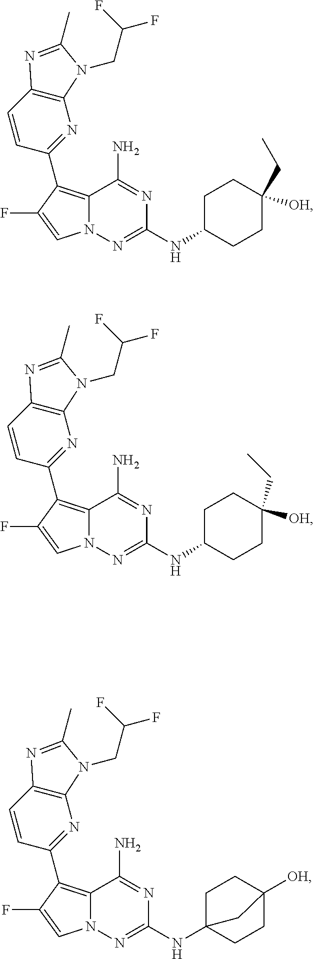 [SMILES: CC[C@]1(O)CC[C@@H](Nc2nc(N)c3c(-c4ccc5nc(C)n(CC(F)F)c5n4)c(F)cn3n2)CC1 ; CC[C@]1(O)CC[C@H](Nc2nc(N)c3c(-c4ccc5nc(C)n(CC(F)F)c5n4)c(F)cn3n2)CC1 ; Cc1nc2ccc(-c3c(F)cn4nc(NC56CCC(O)(CC5)C6)nc(N)c34)nc2n1CC(F)F] 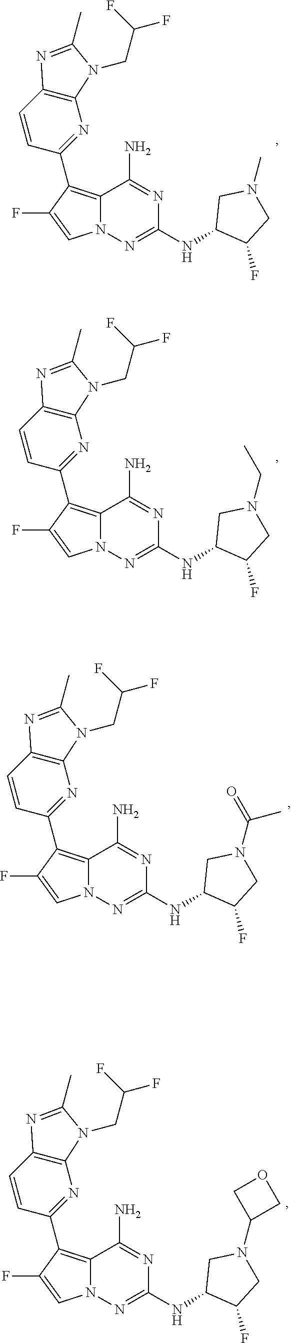 [SMILES: Cc1nc2ccc(-c3c(F)cn4nc(N[C@@H]5CN(C)C[C@@H]5F)nc(N)c34)nc2n1CC(F)F ; CCN1C[C@H](F)[C@H](Nc2nc(N)c3c(-c4ccc5nc(C)n(CC(F)F)c5n4)c(F)cn3n2)C1 ; CC(=O)N1C[C@H](F)[C@H](Nc2nc(N)c3c(-c4ccc5nc(C)n(CC(F)F)c5n4)c(F)cn3n2)C1 ; Cc1nc2ccc(-c3c(F)cn4nc(N[C@@H]5CN(C6COC6)C[C@@H]5F)nc(N)c34)nc2n1CC(F)F] 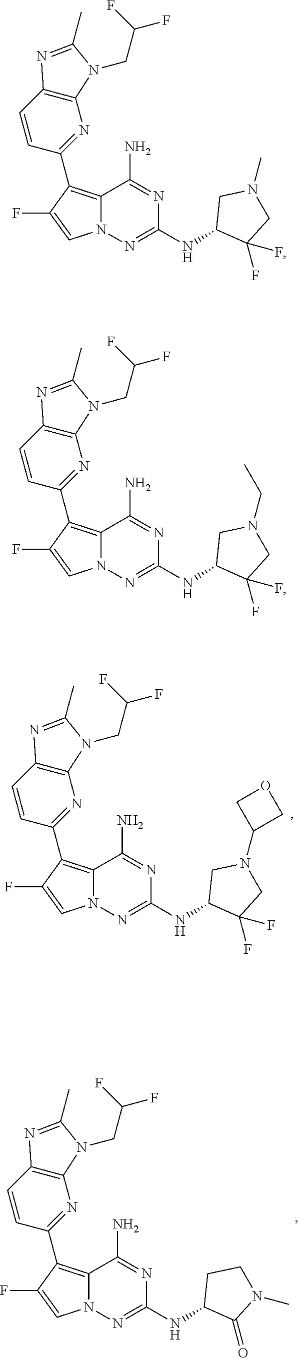 [SMILES: Cc1nc2ccc(-c3c(F)cn4nc(N[C@@H]5CN(C)CC5(F)F)nc(N)c34)nc2n1CC(F)F ; CCN1C[C@@H](Nc2nc(N)c3c(-c4ccc5nc(C)n(CC(F)F)c5n4)c(F)cn3n2)C(F)(F)C1 ; Cc1nc2ccc(-c3c(F)cn4nc(N[C@@H]5CN(C6COC6)CC5(F)F)nc(N)c34)nc2n1CC(F)F ; Cc1nc2ccc(-c3c(F)cn4nc(N[C@@H]5CCN(C)C5=O)nc(N)c34)nc2n1CC(F)F] 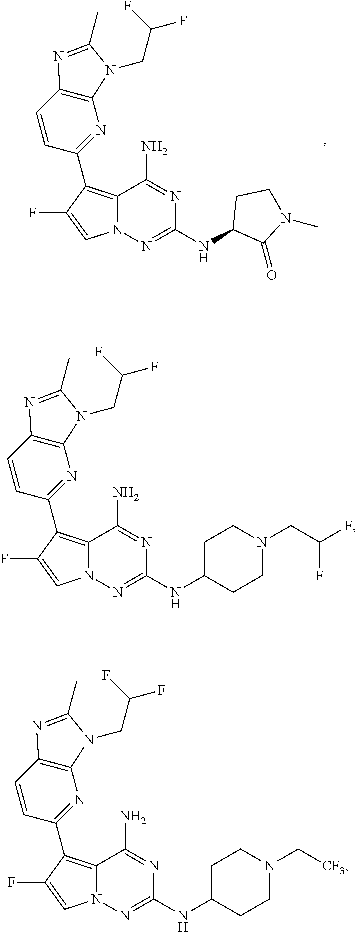 [SMILES: Cc1nc2ccc(-c3c(F)cn4nc(N[C@H]5CCN(C)C5=O)nc(N)c34)nc2n1CC(F)F ; Cc1nc2ccc(-c3c(F)cn4nc(NC5CCN(CC(F)F)CC5)nc(N)c34)nc2n1CC(F)F ; Cc1nc2ccc(-c3c(F)cn4nc(NC5CCN(CC(F)(F)F)CC5)nc(N)c34)nc2n1CC(F)F] 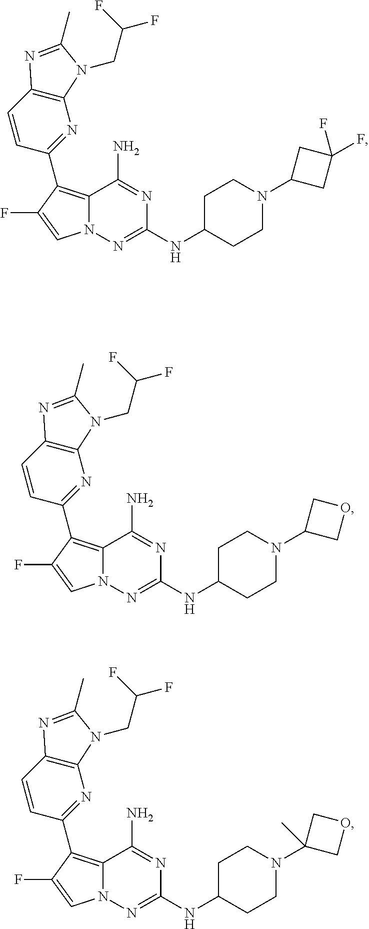 [SMILES: Cc1nc2ccc(-c3c(F)cn4nc(NC5CCN(C6CC(F)(F)C6)CC5)nc(N)c34)nc2n1CC(F)F ; Cc1nc2ccc(-c3c(F)cn4nc(NC5CCN(C6COC6)CC5)nc(N)c34)nc2n1CC(F)F ; Cc1nc2ccc(-c3c(F)cn4nc(NC5CCN(C6(C)COC6)CC5)nc(N)c34)nc2n1CC(F)F] 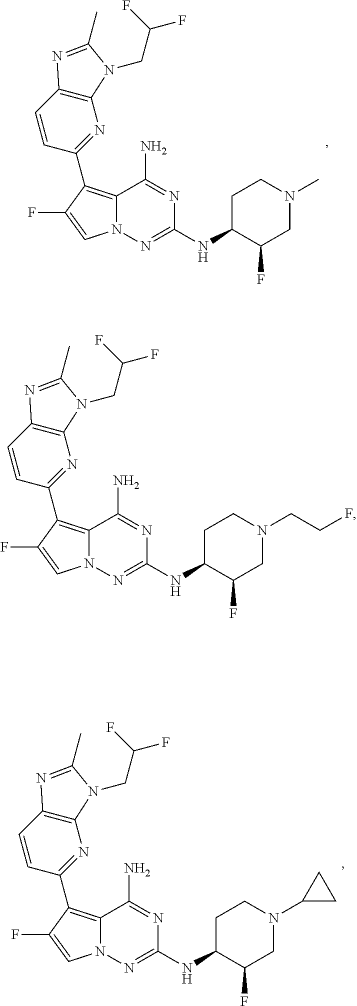 [SMILES: Cc1nc2ccc(-c3c(F)cn4nc(N[C@H]5CCN(C)C[C@H]5F)nc(N)c34)nc2n1CC(F)F ; Cc1nc2ccc(-c3c(F)cn4nc(N[C@H]5CCN(CCF)C[C@H]5F)nc(N)c34)nc2n1CC(F)F ; Cc1nc2ccc(-c3c(F)cn4nc(N[C@H]5CCN(C6CC6)C[C@H]5F)nc(N)c34)nc2n1CC(F)F] 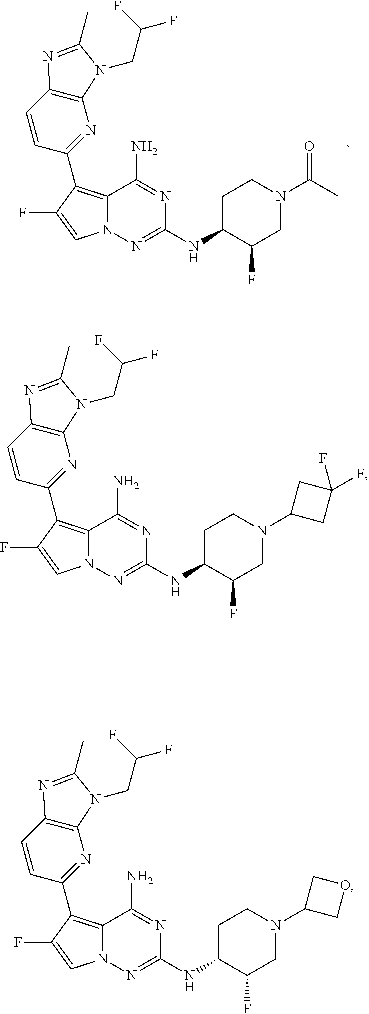 [SMILES: CC(=O)N1CC[C@H](Nc2nc(N)c3c(-c4ccc5nc(C)n(CC(F)F)c5n4)c(F)cn3n2)[C@H](F)C1 ; Cc1nc2ccc(-c3c(F)cn4nc(N[C@H]5CCN(C6CC(F)(F)C6)C[C@H]5F)nc(N)c34)nc2n1CC(F)F ; Cc1nc2ccc(-c3c(F)cn4nc(N[C@@H]5CCN(C6COC6)C[C@@H]5F)nc(N)c34)nc2n1CC(F)F] 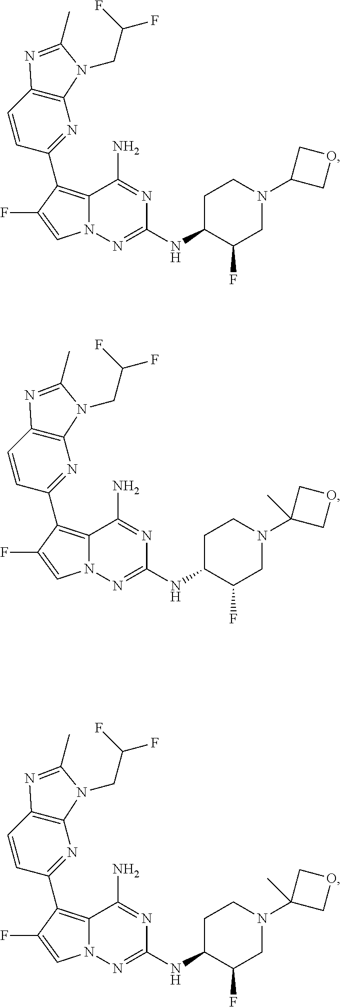 [SMILES: Cc1nc2ccc(-c3c(F)cn4nc(N[C@H]5CCN(C6COC6)C[C@H]5F)nc(N)c34)nc2n1CC(F)F ; Cc1nc2ccc(-c3c(F)cn4nc(N[C@@H]5CCN(C6(C)COC6)C[C@@H]5F)nc(N)c34)nc2n1CC(F)F ; Cc1nc2ccc(-c3c(F)cn4nc(N[C@H]5CCN(C6(C)COC6)C[C@H]5F)nc(N)c34)nc2n1CC(F)F] 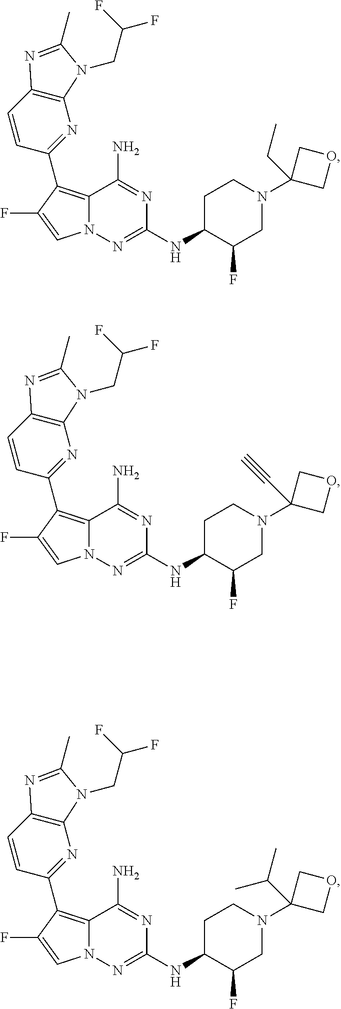 [SMILES: CCC1(N2CC[C@H](Nc3nc(N)c4c(-c5ccc6nc(C)n(CC(F)F)c6n5)c(F)cn4n3)[C@H](F)C2)COC1 ; C#CC1(N2CC[C@H](Nc3nc(N)c4c(-c5ccc6nc(C)n(CC(F)F)c6n5)c(F)cn4n3)[C@H](F)C2)COC1 ; Cc1nc2ccc(-c3c(F)cn4nc(N[C@H]5CCN(C6(C(C)C)COC6)C[C@H]5F)nc(N)c34)nc2n1CC(F)F] 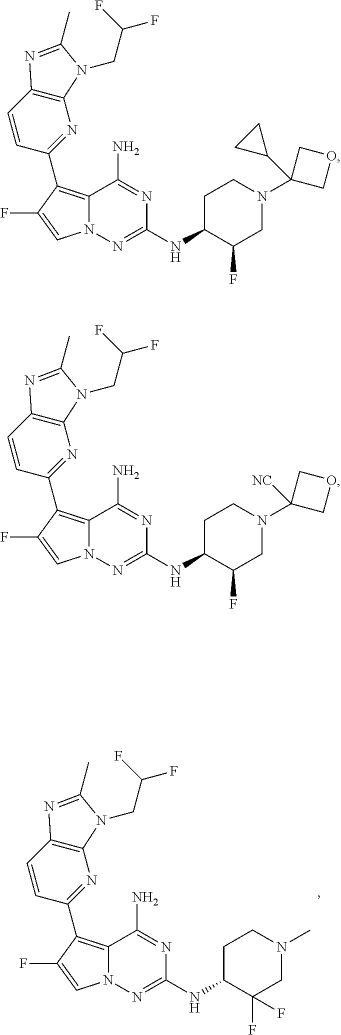 [SMILES: Cc1nc2ccc(-c3c(F)cn4nc(N[C@H]5CCN(C6(C7CC7)COC6)C[C@H]5F)nc(N)c34)nc2n1CC(F)F ; Cc1nc2ccc(-c3c(F)cn4nc(N[C@H]5CCN(C6(C#N)COC6)C[C@H]5F)nc(N)c34)nc2n1CC(F)F ; Cc1nc2ccc(-c3c(F)cn4nc(N[C@@H]5CCN(C)CC5(F)F)nc(N)c34)nc2n1CC(F)F] 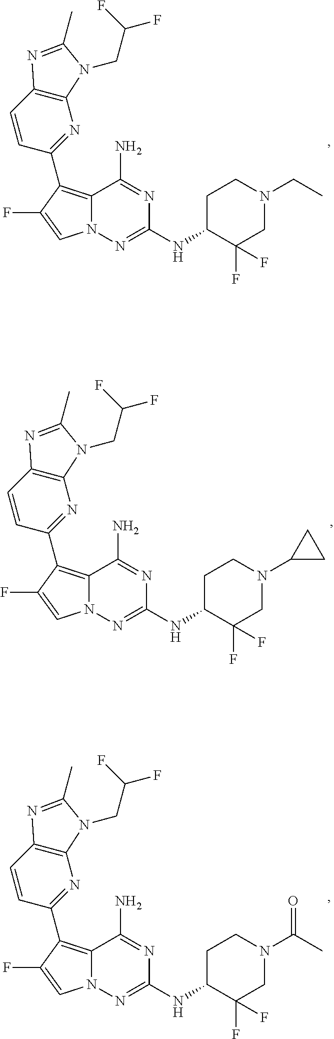 [SMILES: CCN1CC[C@@H](Nc2nc(N)c3c(-c4ccc5nc(C)n(CC(F)F)c5n4)c(F)cn3n2)C(F)(F)C1 ; Cc1nc2ccc(-c3c(F)cn4nc(N[C@@H]5CCN(C6CC6)CC5(F)F)nc(N)c34)nc2n1CC(F)F ; CC(=O)N1CC[C@@H](Nc2nc(N)c3c(-c4ccc5nc(C)n(CC(F)F)c5n4)c(F)cn3n2)C(F)(F)C1] 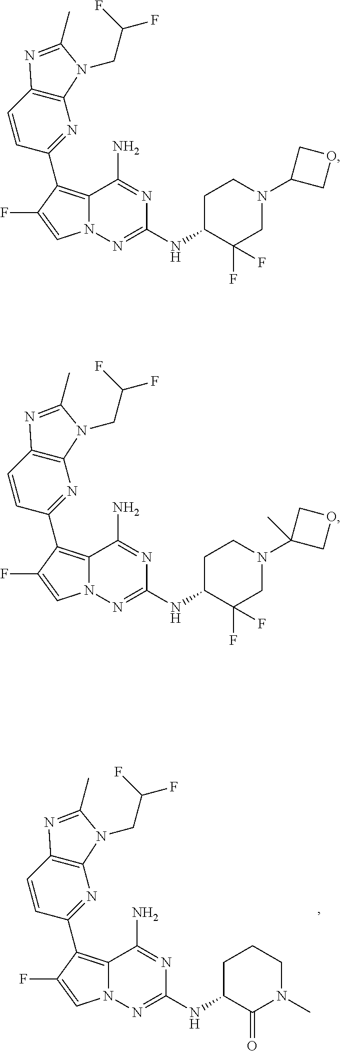 [SMILES: Cc1nc2ccc(-c3c(F)cn4nc(N[C@@H]5CCN(C6COC6)CC5(F)F)nc(N)c34)nc2n1CC(F)F ; Cc1nc2ccc(-c3c(F)cn4nc(N[C@@H]5CCN(C6(C)COC6)CC5(F)F)nc(N)c34)nc2n1CC(F)F ; Cc1nc2ccc(-c3c(F)cn4nc(N[C@@H]5CCCN(C)C5=O)nc(N)c34)nc2n1CC(F)F] 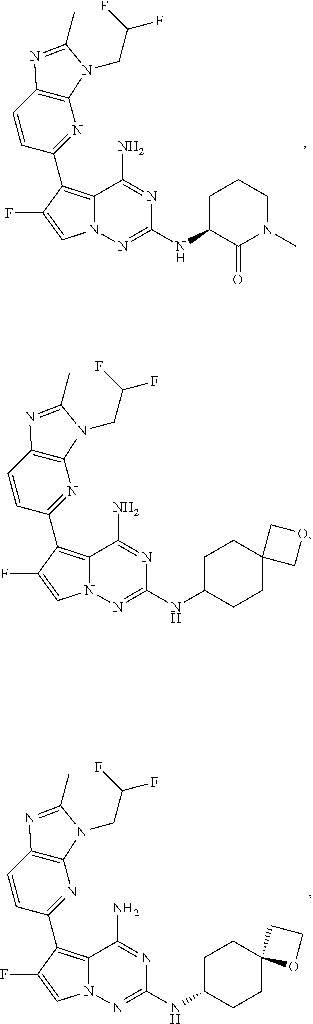 [SMILES: Cc1nc2ccc(-c3c(F)cn4nc(N[C@H]5CCCN(C)C5=O)nc(N)c34)nc2n1CC(F)F ; Cc1nc2ccc(-c3c(F)cn4nc(NC5CCC6(CC5)COC6)nc(N)c34)nc2n1CC(F)F ; Cc1nc2ccc(-c3c(F)cn4nc(N[C@H]5CC[C@@]6(CCO6)CC5)nc(N)c34)nc2n1CC(F)F] 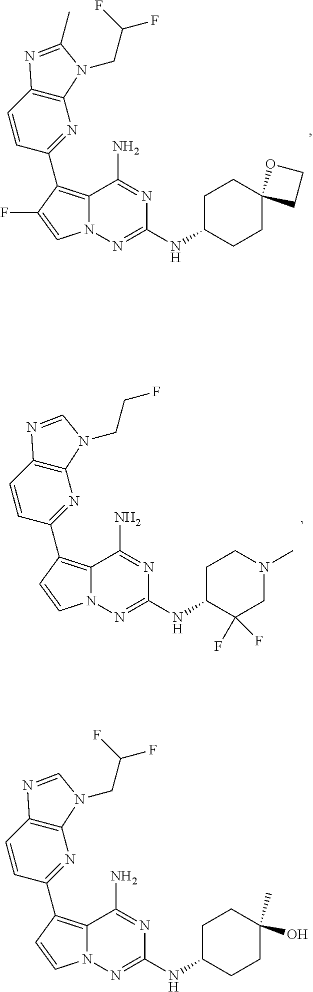 [SMILES: Cc1nc2ccc(-c3c(F)cn4nc(N[C@H]5CC[C@]6(CCO6)CC5)nc(N)c34)nc2n1CC(F)F ; CN1CC[C@@H](Nc2nc(N)c3c(-c4ccc5ncn(CCF)c5n4)ccn3n2)C(F)(F)C1 ; C[C@]1(O)CC[C@H](Nc2nc(N)c3c(-c4ccc5ncn(CC(F)F)c5n4)ccn3n2)CC1] 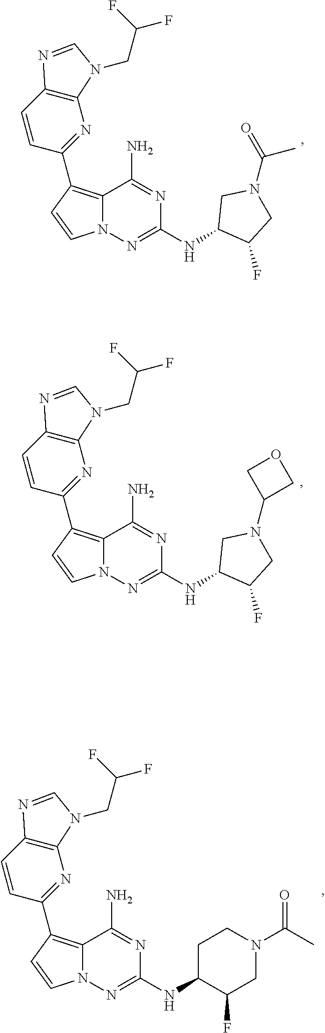 [SMILES: CC(=O)N1C[C@H](F)[C@H](Nc2nc(N)c3c(-c4ccc5ncn(CC(F)F)c5n4)ccn3n2)C1 ; Nc1nc(N[C@@H]2CN(C3COC3)C[C@@H]2F)nn2ccc(-c3ccc4ncn(CC(F)F)c4n3)c12 ; CC(=O)N1CC[C@H](Nc2nc(N)c3c(-c4ccc5ncn(CC(F)F)c5n4)ccn3n2)[C@H](F)C1] 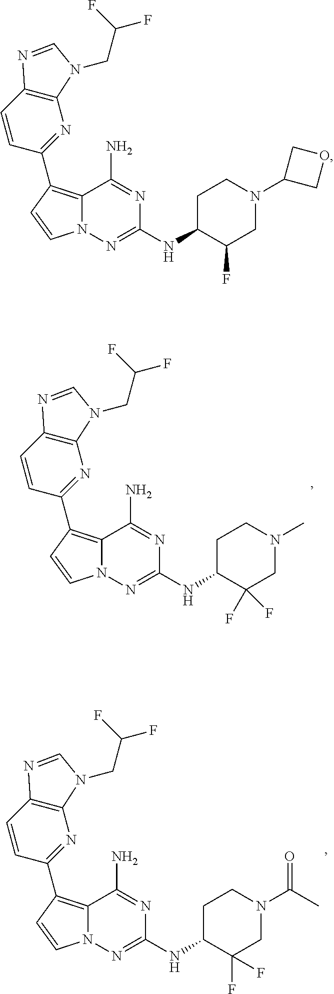 [SMILES: Nc1nc(N[C@H]2CCN(C3COC3)C[C@H]2F)nn2ccc(-c3ccc4ncn(CC(F)F)c4n3)c12 ; CN1CC[C@@H](Nc2nc(N)c3c(-c4ccc5ncn(CC(F)F)c5n4)ccn3n2)C(F)(F)C1 ; CC(=O)N1CC[C@@H](Nc2nc(N)c3c(-c4ccc5ncn(CC(F)F)c5n4)ccn3n2)C(F)(F)C1] 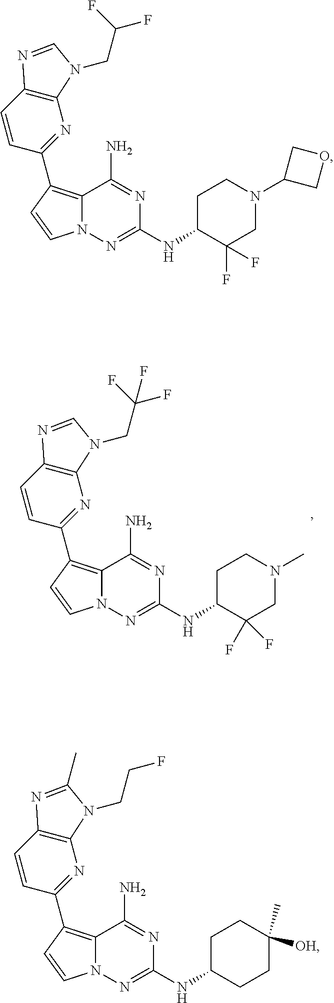 [SMILES: Nc1nc(N[C@@H]2CCN(C3COC3)CC2(F)F)nn2ccc(-c3ccc4ncn(CC(F)F)c4n3)c12 ; CN1CC[C@@H](Nc2nc(N)c3c(-c4ccc5ncn(CC(F)(F)F)c5n4)ccn3n2)C(F)(F)C1 ; Cc1nc2ccc(-c3ccn4nc(N[C@H]5CC[C@](C)(O)CC5)nc(N)c34)nc2n1CCF] 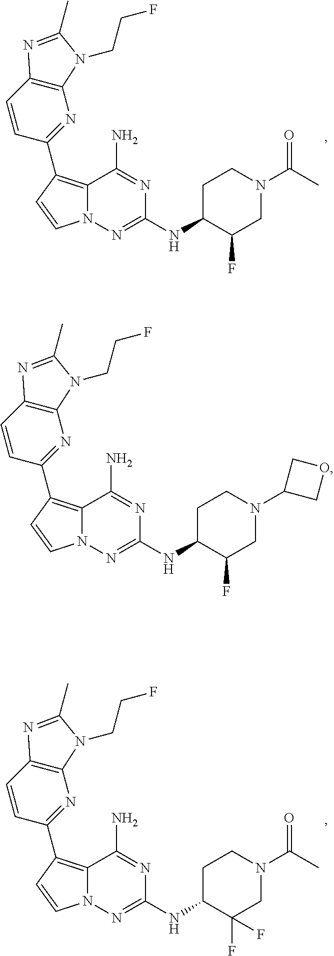 [SMILES: CC(=O)N1CC[C@H](Nc2nc(N)c3c(-c4ccc5nc(C)n(CCF)c5n4)ccn3n2)[C@H](F)C1 ; Cc1nc2ccc(-c3ccn4nc(N[C@H]5CCN(C6COC6)C[C@H]5F)nc(N)c34)nc2n1CCF ; CC(=O)N1CC[C@@H](Nc2nc(N)c3c(-c4ccc5nc(C)n(CCF)c5n4)ccn3n2)C(F)(F)C1] 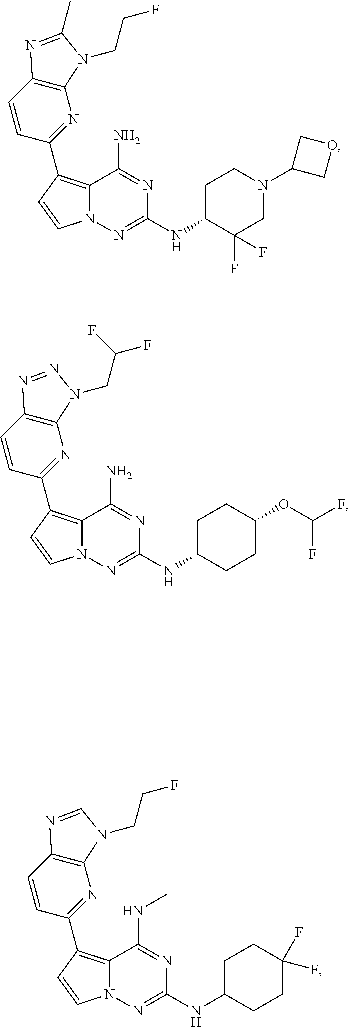 [SMILES: Cc1nc2ccc(-c3ccn4nc(N[C@@H]5CCN(C6COC6)CC5(F)F)nc(N)c34)nc2n1CCF ; Nc1nc(N[C@H]2CC[C@@H](OC(F)F)CC2)nn2ccc(-c3ccc4nnn(CC(F)F)c4n3)c12 ; CNc1nc(NC2CCC(F)(F)CC2)nn2ccc(-c3ccc4ncn(CCF)c4n3)c12] 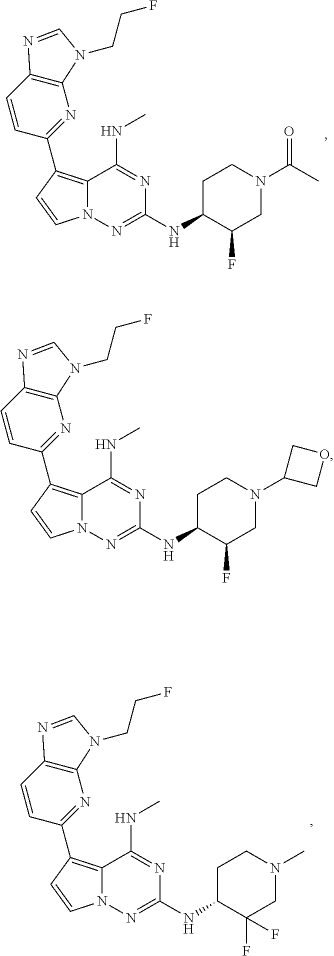 [SMILES: CNc1nc(N[C@H]2CCN(C(C)=O)C[C@H]2F)nn2ccc(-c3ccc4ncn(CCF)c4n3)c12 ; CNc1nc(N[C@H]2CCN(C3COC3)C[C@H]2F)nn2ccc(-c3ccc4ncn(CCF)c4n3)c12 ; CNc1nc(N[C@@H]2CCN(C)CC2(F)F)nn2ccc(-c3ccc4ncn(CCF)c4n3)c12] 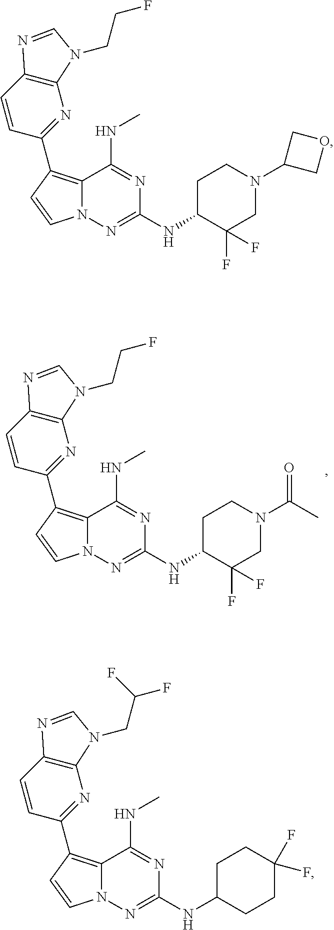 [SMILES: CNc1nc(N[C@@H]2CCN(C3COC3)CC2(F)F)nn2ccc(-c3ccc4ncn(CCF)c4n3)c12 ; CNc1nc(N[C@@H]2CCN(C(C)=O)CC2(F)F)nn2ccc(-c3ccc4ncn(CCF)c4n3)c12 ; CNc1nc(NC2CCC(F)(F)CC2)nn2ccc(-c3ccc4ncn(CC(F)F)c4n3)c12] 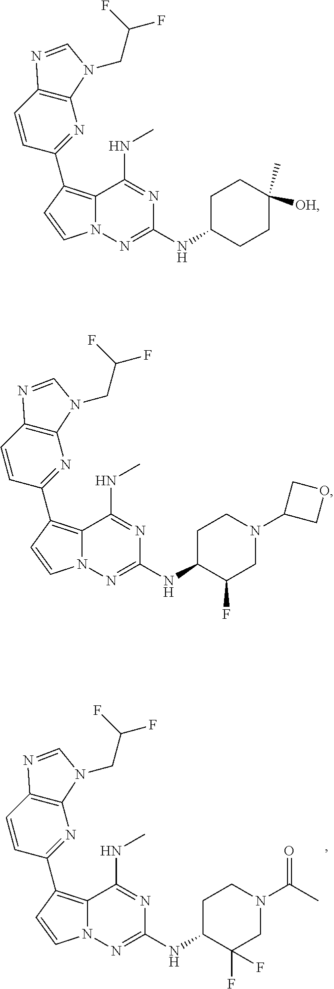 [SMILES: CNc1nc(N[C@H]2CC[C@](C)(O)CC2)nn2ccc(-c3ccc4ncn(CC(F)F)c4n3)c12 ; CNc1nc(N[C@H]2CCN(C3COC3)C[C@H]2F)nn2ccc(-c3ccc4ncn(CC(F)F)c4n3)c12 ; CNc1nc(N[C@@H]2CCN(C(C)=O)CC2(F)F)nn2ccc(-c3ccc4ncn(CC(F)F)c4n3)c12] 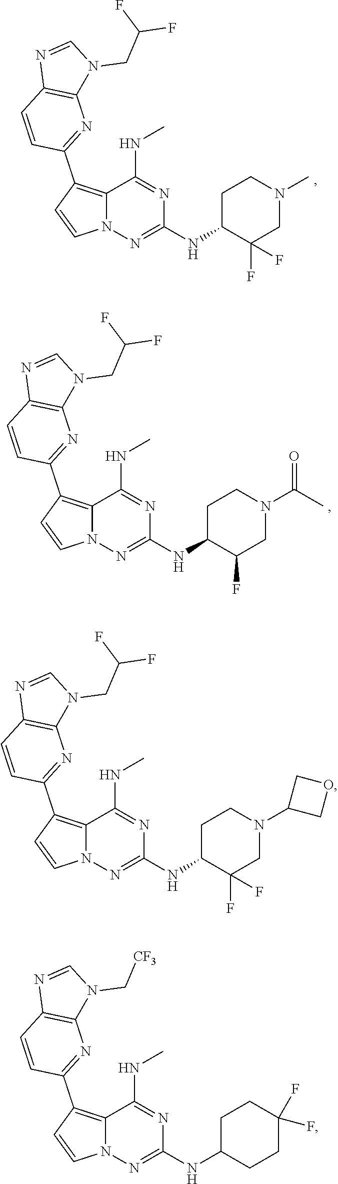 [SMILES: CNc1nc(N[C@@H]2CCN(C)CC2(F)F)nn2ccc(-c3ccc4ncn(CC(F)F)c4n3)c12 ; CNc1nc(N[C@H]2CCN(C(C)=O)C[C@H]2F)nn2ccc(-c3ccc4ncn(CC(F)F)c4n3)c12 ; CNc1nc(N[C@@H]2CCN(C3COC3)CC2(F)F)nn2ccc(-c3ccc4ncn(CC(F)F)c4n3)c12 ; CNc1nc(NC2CCC(F)(F)CC2)nn2ccc(-c3ccc4ncn(CC(F)(F)F)c4n3)c12] 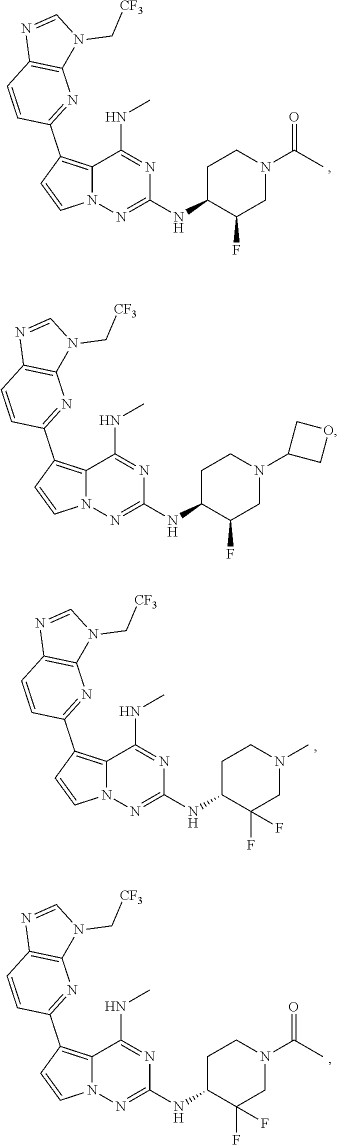 [SMILES: CNc1nc(N[C@H]2CCN(C(C)=O)C[C@H]2F)nn2ccc(-c3ccc4ncn(CC(F)(F)F)c4n3)c12 ; CNc1nc(N[C@H]2CCN(C3COC3)C[C@H]2F)nn2ccc(-c3ccc4ncn(CC(F)(F)F)c4n3)c12 ; CNc1nc(N[C@@H]2CCN(C)CC2(F)F)nn2ccc(-c3ccc4ncn(CC(F)(F)F)c4n3)c12 ; CNc1nc(N[C@@H]2CCN(C(C)=O)CC2(F)F)nn2ccc(-c3ccc4ncn(CC(F)(F)F)c4n3)c12] 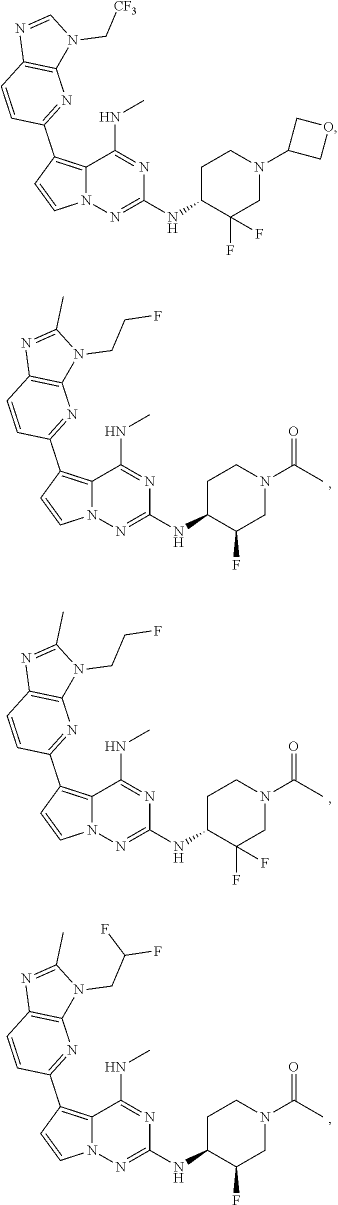 [SMILES: CNc1nc(N[C@@H]2CCN(C3COC3)CC2(F)F)nn2ccc(-c3ccc4ncn(CC(F)(F)F)c4n3)c12 ; CNc1nc(N[C@H]2CCN(C(C)=O)C[C@H]2F)nn2ccc(-c3ccc4nc(C)n(CCF)c4n3)c12 ; CNc1nc(N[C@@H]2CCN(C(C)=O)CC2(F)F)nn2ccc(-c3ccc4nc(C)n(CCF)c4n3)c12 ; CNc1nc(N[C@H]2CCN(C(C)=O)C[C@H]2F)nn2ccc(-c3ccc4nc(C)n(CC(F)F)c4n3)c12] 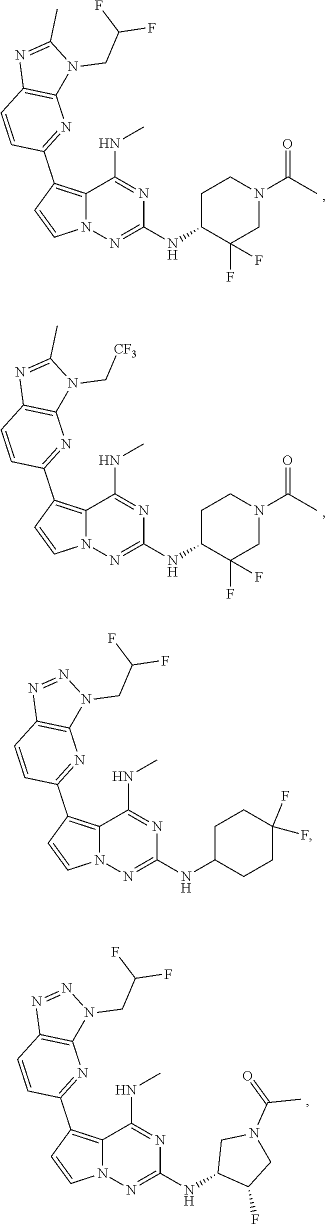 [SMILES: CNc1nc(N[C@@H]2CCN(C(C)=O)CC2(F)F)nn2ccc(-c3ccc4nc(C)n(CC(F)F)c4n3)c12 ; CNc1nc(N[C@@H]2CCN(C(C)=O)CC2(F)F)nn2ccc(-c3ccc4nc(C)n(CC(F)(F)F)c4n3)c12 ; CNc1nc(NC2CCC(F)(F)CC2)nn2ccc(-c3ccc4nnn(CC(F)F)c4n3)c12 ; CNc1nc(N[C@@H]2CN(C(C)=O)C[C@@H]2F)nn2ccc(-c3ccc4nnn(CC(F)F)c4n3)c12] 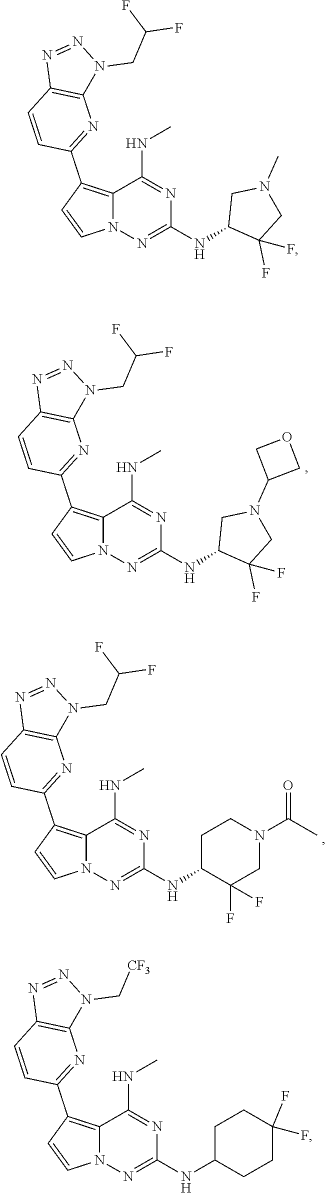 [SMILES: CNc1nc(N[C@@H]2CN(C)CC2(F)F)nn2ccc(-c3ccc4nnn(CC(F)F)c4n3)c12 ; CNc1nc(N[C@@H]2CN(C3COC3)CC2(F)F)nn2ccc(-c3ccc4nnn(CC(F)F)c4n3)c12 ; CNc1nc(N[C@@H]2CCN(C(C)=O)CC2(F)F)nn2ccc(-c3ccc4nnn(CC(F)F)c4n3)c12 ; CNc1nc(NC2CCC(F)(F)CC2)nn2ccc(-c3ccc4nnn(CC(F)(F)F)c4n3)c12] 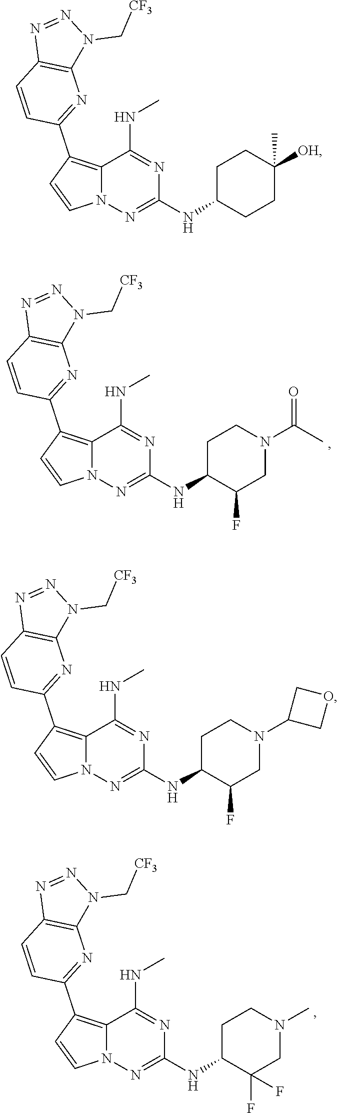 [SMILES: CNc1nc(N[C@H]2CC[C@](C)(O)CC2)nn2ccc(-c3ccc4nnn(CC(F)(F)F)c4n3)c12 ; CNc1nc(N[C@H]2CCN(C(C)=O)C[C@H]2F)nn2ccc(-c3ccc4nnn(CC(F)(F)F)c4n3)c12 ; CNc1nc(N[C@H]2CCN(C3COC3)C[C@H]2F)nn2ccc(-c3ccc4nnn(CC(F)(F)F)c4n3)c12 ; CNc1nc(N[C@@H]2CCN(C)CC2(F)F)nn2ccc(-c3ccc4nnn(CC(F)(F)F)c4n3)c12] 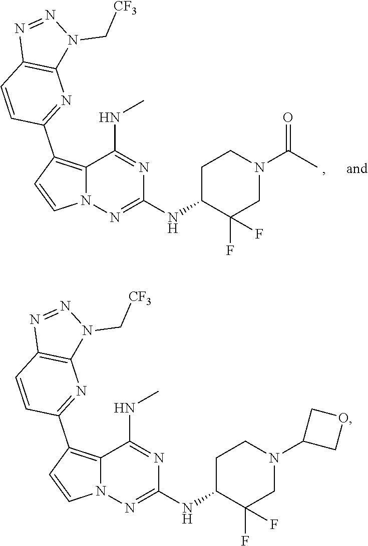 [SMILES: CNc1nc(N[C@@H]2CCN(C(C)=O)CC2(F)F)nn2ccc(-c3ccc4nnn(CC(F)(F)F)c4n3)c12 ; CNc1nc(N[C@@H]2CCN(C3COC3)CC2(F)F)nn2ccc(-c3ccc4nnn(CC(F)(F)F)c4n3)c12]
or a pharmaceutically acceptable salt thereof.

21. A pharmaceutical composition comprising a therapeutically effective amount of a compound of claim 1, or a pharmaceutically acceptable salt thereof, and a pharmaceutically acceptable excipient.

22. A pharmaceutical composition comprising a therapeutically effective amount of a compound of claim 20, or a pharmaceutically acceptable salt thereof, and a pharmaceutically acceptable excipient.

23. A method of treating a disorder or disease in a patient, wherein the disorder or disease is selected from the group consisting of: a neurological disorder, diabetes, and cancer, the method comprising administering to the patient a therapeutically effective amount of a compound of claim 1, or a pharmaceutically acceptable salt.

24. A method of treating a disorder or disease in a patient, wherein the disorder or disease is selected from the group consisting of: a neurological disorder, diabetes, and cancer, the method comprising administering to the patient a therapeutically effective amount of a compound of claim 20, or a pharmaceutically acceptable salt.

25. The method of claim 23, wherein the cancer is selected from the group consisting of: brain tumors, glioblastoma, ovarian, breast, head and neck squamous cell carcinoma, hepatocellular carcinoma, pancreatic cancer, acute lymphoblastic leukemia, acute megakaryoblastic leukemia, and chronic myeloid leukemia.

26. The method of claim 24, wherein the cancer is selected from the group consisting of: brain tumors, glioblastoma, ovarian, breast, head and neck squamous cell carcinoma, hepatocellular carcinoma, pancreatic cancer, acute lymphoblastic leukemia, acute megakaryoblastic leukemia, and chronic myeloid leukemia.

27. The method of claim 23, wherein the disorder or disease is a neurological disorder, wherein the neurological disorder is selected from the group consisting of: Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, Pick disease, progressive supranuclear palsy, corticobasal degeneration, argyrophilic grain disease, globular glial tauopathies, primary age-related tauopathy, neurofibrillary tangle dementia, chronic traumatic encephalopathy (CTE), frontotemporal lobar degeneration with tau inclusions (FTLD-tau), and aging-related tau astrogliopathy.

28. The method of claim 24, wherein the disorder or disease is a neurological disorder, wherein the neurological disorder is selected from the group consisting of: Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, Pick disease, progressive supranuclear palsy, corticobasal degeneration, argyrophilic grain disease, globular glial tauopathies, primary age-related tauopathy, neurofibrillary tangle dementia, chronic traumatic encephalopathy (CTE), frontotemporal lobar degeneration with tau inclusions (FTLD-tau), and aging-related tau astrogliopathy.

29. The method of claim 27, wherein the disorder or disease is Alzheimer's disease.

30. The method of claim 28, wherein the disorder or disease is Alzheimer's disease.

31. The method of claim 23, wherein the patient is a human.

32. The method of claim 24, wherein the patient is a human.
